(19) United States
(12) Patent Application Publication
(10) Pub. No.: US 2025/0011372 A1
(43) Pub. Date: Jan. 9, 2025

(54) AAV CAPSID VARIANTS AND USES THEREOF

(71) Applicant: VOYAGER THRAPEUTICS, INC., Lexington, MA (US)

(72) Inventors: Mathieu Emmanuel Nonnenmacher, Boston, MA (US); Matthew Alan Child, Ashland, MA (US); Jinzhao Hou, Lexington, MA (US); Jiangyu Li, Watertown, MA (US); Shaoyong Li, Hopkinton, MA (US); Tyler Christopher Moyer, Boston, MA (US); Wei Wang, Arlington, MA (US); Matteo Placido Placidi, Arlington, MA (US); Robert H. Vass, Gardner, MA (US); Brett Hoffman, Braintree, MA (US); Elisabeth Knoll, Melrose, MA (US); Yanqun Shu, Winchester, MA (US); Nilesh Navalkishor Pande, North Grafton, MA (US); Jeffrey Scott Thompson, Stoneham, MA (US)

(21) Appl. No.: 18/710,005

(22) PCT Filed: Nov. 16, 2022
(86) PCT No.: PCT/US2022/079963

§ 371 Date: May 14, 2024

Related U.S. Application Data
(60) Provisional application No. 63/414,377, filed on Oct. 7, 2022.
(60) Provisional application No. 63/286,545, filed on Dec. 6, 2021.
(60) Provisional application No. 63/280,417, filed on Nov. 17, 2021.

(51) Int. Cl. C07K 14/005; C12N 15/86

(52) U.S. Cl. CPC C07K 14/005; C12N 15/86; C12N 2750/14122; C12N 2750/14143; C12N 2750/14145

(57) ABSTRACT

The disclosure relates to compositions, formulations, and methods for the preparation, use, and/or formulation of adeno-associated virus capsid protein variants.

### RELATED APPLICATIONS

This application claims priority to U.S. Provisional Application No. 63/280,417 filed on Nov. 17, 2021, U.S. Provisional Application No. 63/286,545 filed on Dec. 6, 2021, and U.S. Provisional Application No. 63/414,377 filed on Oct. 7, 2022; the entire contents of each of which are hereby incorporated by reference in their entirety.

### SEQUENCE LISTING

The instant application contains a Sequence Listing which has been submitted electronically in XML format and is hereby incorporated by reference in its entirety. Said XML copy, created on Nov. 10, 2022, is named V2071-1132PCT_SL.xml and is 3,391,526 bytes in size.

### FIELD OF THE DISCLOSURE

The disclosure relates to compositions, formulations, and methods for the preparation, use, and/or formulation of adeno-associated virus capsid proteins and variants thereof.

### BACKGROUND

Gene delivery to the adult central nervous system (CNS) remains a significant challenge in gene therapy. Engineered adeno-associated virus (AAV) capsids with improved brain tropism represent an attractive solution to the limitations of CNS delivery.

AAV-derived vectors are promising tools for clinical gene transfer because of their non-pathogenic nature, their low immunogenic profile, low rate of integration into the host genome and long-term transgene expression in non-dividing cells. However, the transduction efficiency of AAV natural variants in certain organs is too low for clinical applications, and capsid neutralization by pre-existing neutralizing antibodies may prevent treatment of a large proportion of patients. For these reasons, considerable efforts have been devoted to obtaining capsid variants with enhanced properties. Of many approaches tested so far, significant advances have resulted from directed evolution of AAV capsids using in vitro or in vivo selection of capsid variants created by capsid sequence randomization using either error-prone PCR, shuffling of various parent serotypes, or insertion of fully randomized short peptides at defined positions.

Attempts at providing AAV capsids with improved properties, e.g., improved tropism to a target cell or tissue upon systemic administration, have met with limited success. As such, there is a need for improved methods of producing AAV capsids and resulting AAV capsids for delivery of a payload of interest to a target cell or tissue, e.g., a CNS cell or tissue, or a muscle cell or tissue.

### SUMMARY OF THE DISCLOSURE

The present disclosure pertains at least in part, to compositions, formulations, and methods for the production and use of an AAV particle comprising an AAV capsid polypeptide, e.g., an AAV capsid variant. In some embodiments, the AAV capsid variant has an enhanced tropism for a tissue or a cell, e.g., a CNS tissue, a CNS cell, a muscle tissue, or a muscle cell. Said tropism can be useful for delivery of a payload, e.g., a payload described herein to a cell or tissue, for the treatment of a disorder, e.g., a neurological or a neurodegenerative disorder, a muscular or a neuromuscular disorder, or a neuro-oncological disorder.

Accordingly, in one aspect, the present disclosure provides an AAV capsid variant, comprising an amino sequence comprising the following formula: [N1]-[N2], wherein: (i) [N1] comprises X1, X2, X3, X4, and X5, wherein: (a) position X1 is: P, Q, A, H, K, L, R, S, or T; (b) position X2 is: L, I, V, H, or R; (c) position X3 is: N, D, I, K, or Y; (d) position X4 is: G, A, C, R, or S; and (e) position X5 is: A, S, T, G, C, D, N, Q, V, or Y; and (ii) [N2] comprises the amino acid sequence of VHLY (SEQ ID NO: 4680), VHIY (SEQ ID NO: 4681), VHVY (SEQ ID NO: 4682), or VHHY (SEQ ID NO: 4683); and/or an amino acid modification, e.g., a conservative substitution, of any of the aforesaid amino acids in (i) and/or (ii). In some embodiments, (a) position X1 is P; (b) position X2 is L; (c) position X3 is N, D, I, K, or Y; (d) position X4 is G; and (e) position X5 is A. In some embodiments, [N2] is or comprises VHLY (SEQ ID NO: 4680). In some embodiments, the AAV capsid variant further comprises one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138. In some embodiments, the AAV capsid variant further comprises one, two, or all of an amino acid other than V at position 596 (e.g., D, F, G, L, A, E, or I), an amino acid other than Q at position 597 (e.g., P, K, R, H, E, or L), and/or an amino acid other than N at position 598 (e.g., T, K, H, D, Y, S, I, or P), relative to a reference sequence numbered according to the amino acid sequence of SEQ ID NO: 138.

In another aspect, the present disclosure provides an AAV capsid variant comprising one, two, three, four, or all of: (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722); (ii) an [N2] wherein [N2] is or comprises: VHLY (SEQ ID NO: 4680), VHVY (SEQ ID NO: 4682), VPLY (SEQ ID NO: 4723), VNLY (SEQ ID NO: 4724), VHRY (SEQ ID NO: 4725), VHIY (SEQ ID NO: 4681), VHHY (SEQ ID NO: 4683), FHLY (SEQ ID NO: 4726), LHLY (SEQ ID NO: 4727), DHLY (SEQ ID NO: 4728), VQLY (SEQ ID NO: 4729), IHLY (SEQ ID NO: 4730), VDLY (SEQ ID NO: 4731), AHLY (SEQ ID NO: 4732), VLLY (SEQ ID NO: 4733), GHLY (SEQ ID NO: 4734), VRLY (SEQ ID NO: 4735), or VYLY (SEQ ID NO: 4736); (iii) an [N3] wherein [N3] is or comprises: AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751); (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or wherein the AAV capsid variant comprises an amino acid modification, e.g., a conservative substitution, of any of the aforesaid amino acids in (i)-(v).

In yet another aspect, the present disclosure provides an AAV capsid variant comprising one, two, three, four, or all of: (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), or PLDSS (SEQ ID NO: 4705); (ii) an [N2] wherein [N2] is or comprises: VHLY (SEQ ID NO: 4680) or VHVY (SEQ ID NO: 4682); (iii) an [N3] wherein [N3] is or comprises: AQAQ (SEQ ID NO: 4737), SQAQ (SEQ ID NO: 4738), AQPQ (SEQ ID NO: 4739), or AQSQ (SEQ ID NO: 4740); (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD; and/or wherein the AAV capsid variant comprises an amino acid modification, e.g., a conservative substitution, of any of the aforesaid amino acids in (i)-(v).

In another aspect, the present disclosure provides an AAV capsid variant comprising [A][B], wherein [A] comprises the amino acid sequence of PLNGA (SEQ ID NO: 3679), and [B] comprises X1, X2, X3, X4, wherein: (i) X1 is: V, I, L, A, F, D, or G; (ii) X2 is: H, N, Q, P, D, L, R, or Y; (iii) X3 is: L, H, I, R, or V; and (iv) X4 is Y; and/or wherein the AAV capsid variant comprises an amino acid modification, e.g., a conservative substitution, of any of the aforesaid amino acids in (i)-(iv). In some embodiments, the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.

In yet another aspect, the present disclosure provides an AAV capsid variant, comprising PLNGAVHLY (SEQ ID NO: 3648) and optionally wherein the AAV capsid variant further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138.

In another aspect, the present disclosure provides an AAV capsid variant comprising an amino sequence comprising the amino acid sequence of PLNGAVHLY (SEQ ID NO: 3648); and which further comprises one, two, three, or all of: (i) the amino acid at position 593, relative to a reference sequence numbered according to the amino acid sequence of SEQ ID NO: 138, is: T, A, L, R, V, C, I, K, M, N, P, Q, or S; (ii) the amino acid at position 594, relative to a reference sequence numbered according to the amino acid sequence of SEQ ID NO: 138, is: G, M, S, A, Q, V, T, L, P, H, K, N, I, Y, or R; and/or (iii) the amino acid at position 595, relative to a reference sequence numbered according to the amino acid sequence of SEQ ID NO: 138: W, S, P, T, A, G, L, Q, H, N, R, K, V, E, F, M, C, or Y; optionally, provided that the amino acids at positions 593-595, relative to a reference sequence numbered according to the amino acid sequence of SEQ ID NO: 138, does not comprise the amino acid sequence of TGW.

In yet another aspect, the present disclosure provides an AAV capsid variant comprising X1-X2-X3-X4-X5-X6-X7-X8-X9-X10-X11-X12-X13-X14-X15-X16-X17-X18-X19, wherein (i) X1 is: P, A, D, E, F, G, H, K, L, N, Q, R, S, T, or V; (ii) X2 is: L, D, E, F, H, I, M, N, P, Q, R, S, or V; (iii) X3 is: N, A, D, E, G, H, I, K, Q, S, T, V, or Y; (iv) X4 is: G, A, C, D, E, P, Q, R, S, T, V, or W; (v) X5 is: A, C, D, E, F, G, H, I, K, N, P, Q, R, S, T, V, W, or Y; (vi) X6 is: V, A, C, D, E, F, G, H, I, K, L, M, N, Q, R, S, T, or Y; (vii) X7 is: H, A, D, E, G, I, K, L, M, N, P, Q, R, S, T, V, or Y; (viii) X8 is: L, A, D, E, F, G, H, I, K, M, N, P, Q, R, S, T, V, or Y; (ix) X9 is: Y, A, C, D, E, F, G, H, I, K, L, M, N, Q, R, S, T, V, or W; (x) X10 is: A, C, D, E, F, G, H, I, K, L, N, P, Q, R, S, T, V, or; Y; (xi) X11 is: Q, A, D, E, H, K, L, P, R, or T; (xii) X12 is: A, D, E, G, H, L, N, P, Q, R, S, T, or V; (xiii) X13 is: Q, E, H, K, L, P, R, or T; (xiv) X14 is: T, A, C, D, E, F, G, H, I, K, L, M, N, P, Q, R, S, V, W, or Y; (xv) X15 is: G, A, C, D, E, F, H, I, K, L, M, N, P, Q, R, S, T, V, W, or Y; (xvi) X16 is: W, A, C, D, E, F, G, H, I, K, L, M, N, P, Q, R, S, T, V, or Y; (xvii) X17 is: V, A, D, E, F, G, H, I, or L; (xviii) X18 is: Q, E, H, K, L, P, or R; and/or (xix) X19 is: N, D, H, I, K, P, S, T, or Y.

In yet another aspect, the present disclosure provides an AAV capsid variant comprising an amino sequence comprising the following formula: [N1]-[N2], wherein: (i) [N1] comprises the amino acid sequence of PLNG (SEQ ID NO: 3678); and (ii) [N2] comprises X1, X2, and X3, wherein: (a) position X1 is: A, V, T, or G; (b) position X2 is: R, K, Q, G, or V; and (c) position X3 is: H, A, M, S, T, Q, or Y, or; and/or an amino acid modification, e.g., a conservative substitution, of any of the aforesaid amino acids in (i) and/or (ii).

In yet another aspect, the present disclosure provides AAV capsid variant comprising one, two, or all of: (i) an [N1], wherein [N1] is or comprises: PLNN (SEQ ID NO: 4752), PLNG (SEQ ID NO: 3678), PSAR (SEQ ID NO: 4753), TLNG (SEQ ID NO: 4754), PLNM (SEQ ID NO: 4755), SLNG (SEQ ID NO: 4756), SING (SEQ ID NO: 4757), ALNG (SEQ ID NO: 4758), PLNL (SEQ ID NO: 4759), PGRQ (SEQ ID NO: 4760), or LVNS (SEQ ID NO: 4761); (ii) an [N2] wherein [N2] is or comprises: PGH, VKA, ARM, VKM, VRA, VRS, TRM, VRT, VRM, AKM, VKS, VQM, AVH, TRS, VRQ, AQM, VKY, ART, AGA, VQA, VKT, PVH, GVH, AGH, VGH, TGH, or TVR; and/or (iii) an [N3] wherein [N3] is or comprises: LY, IY, LN, DY, LS, or VS; and/or wherein the AAV capsid variant comprises an amino acid modification, e.g., a conservative substitution, of any of the aforesaid amino acids in (i)-(v).

In yet another aspect, the present disclosure provides AAV capsid variant, comprising an amino sequence comprising the following formula: [B]-[C], wherein: (i) [B] comprises X1, X2, and X3, wherein: (a) position X1 is: P, Q, A, H, K, L, R, S, or T; (b) position X2 is: L, I, V, H, or R; and (c) position X3 is: N, D, I, K, or Y; and (ii) [C] comprises the amino acid sequence of LY.

In another aspect, the present disclosure provides an AAV capsid variant comprising one, two, or all of: (i) an [A], wherein [A] is or comprises PLNN (SEQ ID NO: 4752), PLNG (SEQ ID NO: 3678), PSAR (SEQ ID NO: 4753), PLNM (SEQ ID NO: 4755), SLNG (SEQ ID NO: 4756), SING (SEQ ID NO: 4757), PLNL (SEQ ID NO: 4759), or PGRQ (SEQ ID NO: 4760); (ii) a [B], wherein [B] is or comprises PGH, VKA, VKM, VRA, VRS, TRM, VRT, VRM, ARM, AKM, VKS, VQM, AVH, TRS, VRQ, AQM, VKY, VQA, VKT, PVH, VGH, or TGH; and/or (iii) a [C] wherein [C] is or comprises LY.

In yet another aspect, the present disclosure provides an AAV capsid variant comprising X1-X2-X3-X4-X5-X6-X7-X8-X9, wherein: (i) X1 is: P, T, S, A, or L; (ii) X2 is: L, S, I, G, or V; (iii) X3 is: N, A, or R; (iv) X4 is: N, G, R, M, L, Q, or S; (v) X5 is: P, V, A, T, or G; (vi) X6 is: G, K, R, Q, or V; (vii) X7 is: H, A, M, S, T, Q, Y, or R; (viii) X8 is: L, I, D, or V; and (ix) X9 is: Y, N, or S.

In yet another aspect, the present disclosure provides an AAV capsid variant comprising (a) the amino acid sequence of any one of the sequences provided in Tables 1A, 1B, 10, or 20; (b) an amino acid sequence comprising at least 3, 4, 5, 6, 7, 8, 9, 10, 11, 12, 13, 14, 15, 16, 17, or 18 consecutive amino acids from any one of the sequences provided in Tables 1A, 1B, 10, or 20; or (c) an amino acid sequence comprising at least one, two, or three but no more than four different amino acids, relative to any one of the amino acid sequences provided in Tables 1A, 1B, 10, or 20; or (d) an amino acid sequence comprising at least one, two, or three but no more than four modifications, e.g., substitutions (e.g., conservative substitutions), insertions, or deletions, relative to the amino acid sequence of any one of the sequences provided in Tables 1A, 1B, 10, 20. In some embodiments, the AAV capsid variant does not comprise the amino acid sequence of TGW at positions 593-595, relative to a reference sequence numbered according to the amino acid sequence of SEQ ID NO: 138.

In yet another aspect, the present disclosure provides an AAV capsid variant comprising (a) the amino acid sequence of any one of SEQ ID NOs: 139-1138; (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; or (c) an amino acid sequence comprising at least one, two, or three but no more than four different amino acids, relative to the amino acid sequence of any one of SEQ ID NOs: 139-1138; (d) an amino sequence comprising at least one, two, or three but no more than four modifications, e.g., substitutions (e.g., conservative substitutions), insertions, or deletions, relative to the amino acid sequence of any one of SEQ ID NOs: 139-1138. In some embodiments, the AAV capsid variant does not comprise the amino acid sequence of TGW at positions 593-595, relative to a reference sequence numbered according to the amino acid sequence of SEQ ID NO: 138.

In yet another aspect, the present disclosure provides an AAV capsid variant comprising: (a) the amino acid sequence of any one of SEQ ID NOs: 139-476; (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or (c) an amino acid sequence comprising at least one, two, or three but no more than four different amino acids, relative to the amino acid sequence of any one of SEQ ID NOs: 139-476; (d) an amino sequence comprising at least one, two, or three but no more than four modifications, e.g., substitutions (e.g., conservative substitutions), insertions, or deletions, relative to the amino acid sequence of any one of SEQ ID NOs: 139-476. In some embodiments, the AAV capsid variant does not comprise the amino acid sequence of TGW at positions 593-595, relative to a reference sequence numbered according to the amino acid sequence of SEQ ID NO: 138.

In yet another aspect, the present disclosure provides an AAV capsid variant comprising (a) the amino acid sequence of any one of the amino acid sequences provided in Table 1B; (b) an amino acid sequence comprising at least 3, 4, 5, 6, 7, 8, 9, 10, 11, 12, 13, 14, 15, 16, 17, or 18 consecutive amino acids from any one of the amino acid sequences provided in Table 1B; or (c) an amino acid sequence comprising at least one, two, or three but no more than four different amino acids, relative to the amino acid sequence of any one of the amino acid sequences provided in Table 1B; (d) an amino sequence comprising at least one, two, or three but no more than four modifications, e.g., substitutions (e.g., conservative substitutions), insertions, or deletions, relative to the amino acid sequence of any one of the amino acid sequences provided in Table 1B. In some embodiments, the AAV capsid variant does not comprise the amino acid sequence of TGW at positions 593-595, relative to a reference sequence numbered according to the amino acid sequence of SEQ ID NO: 138.

In yet another aspect, the present disclosure provides an AAV capsid variant comprising (a) the amino acid sequence of any one of SEQ ID NOs: 1139-1172; (b) an amino acid sequence comprising at least 3, 4, 5, 6, 7, 8, 9, 10, 11, 12, 13, 14, or 15 consecutive amino acids from any one of SEQ ID NOs: 1139-1172; or (c) an amino acid sequence comprising at least one, two, or three but no more than four different amino acids, relative to the amino acid sequence of any one of SEQ ID NOs: 1139-1172; (d) an amino sequence comprising at least one, two, or three but no more than four modifications, e.g., substitutions (e.g., conservative substitutions), insertions, or deletions, relative to the amino acid sequence of any one of SEQ ID NOs: 1139-1172.

In another aspect, the present disclosure provides an AAV capsid polypeptide, e.g., an AAV capsid variant, comprising: the amino acid sequence of any of SEQ ID NO: 1725-3622 or 3648-3659; an amino acid sequence comprising no more than four modifications, e.g., substitutions, relative to the amino acid sequence of any of SEQ ID NO: 1725-3622 or 3648-3659; or at least 3, 4, 5, 6, 7, 8, or 9 consecutive amino acids from the amino acid sequence of any of SEQ ID NO: 1725-3622 or 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138. In some embodiments, the AAV capsid variant comprises the amino acid sequence of any one of SEQ ID NOs: 5, 8, 3636-3647, or an amino acid sequence with at least 80% (e.g., at least about 85, 90, 95, 96, 97, 98, or 99%) sequence identity thereto.

In yet another aspect, the present disclosure provides a peptide comprising (a) the amino acid sequence of any of the sequences provided in Tables 1A, 1B, 10, or 20; (b) an amino acid sequence comprising at least 3, 4, 5, 6, 7, 8, 9, 10, 11, 12, 13, 14, 15, 16, 17, 18, 19, 20 consecutive amino acids from any one of the sequences provided in Tables 1A, 1B, 10, or 20; (c) an amino acid sequence comprising at least one, two, or three but no more than four different amino acids relative to the amino acid sequence of any one of the sequences provided in Tables 1A, 1B, 10, or 20; or (d) an amino acid sequence comprising at least one, two, or three but no more than four modifications, e.g., substitutions (e.g., conservative substitutions), relative to the amino acid sequence of any one of the sequences provided in Tables 1A, 1B, 10, or 20. In some embodiments, the amino acid sequence is not PLN, PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).

In another aspect, the present disclosure provides a peptide comprising: the amino acid sequence of any of SEQ ID NO: 1725-3622 or 3648-3659; an amino acid sequence comprising no more than four modifications, e.g., substitutions, relative to the amino acid sequence of any of SEQ ID NO: 1725-3622 or 3648-3659; or 3, 4, 5, 6, 7, 8, or 9 consecutive amino acids from the amino acid sequence of any of SEQ ID NO: 1725-3622 or 3648-3659. In some embodiments, the peptide is encoded by the nucleotide sequence of any one of SEQ ID NOs: 3660-3671, or a nucleic acid sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications but no more than ten modifications of the nucleotide sequences of any of SEQ ID NOs: 3660-3671. In some embodiments, the nucleotide sequence encoding the peptide comprises the nucleotide sequence of any one of SEQ ID NOs: 3660-3671, or a nucleic acid sequence substantially identical (e.g., having at least 70%, 75%, 80%, 85%, 90%, 92%, 95%, 97%, 98%, or 99% sequence identity) thereto, or a nucleotide sequence comprising at least one, two, three, four, five, six, or seven modifications but no more than ten modifications of the nucleotide sequences of any of SEQ ID NOs: 3660-3671.

In another aspect, the present disclosure provides a polynucleotide encoding an AAV capsid variant comprising: (a) the amino acid sequence of any one of SEQ ID NOs: 139-1138; (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; or (c) an amino acid sequence comprising at least one, two, or three but no more than four different amino acids, relative to the amino acid sequence of any one of SEQ ID NOs: 139-1138; (d) an amino sequence comprising at least one, two, or three but no more than four modifications, e.g., substitutions (e.g., conservative substitutions), relative to the amino acid sequence of any one of SEQ ID NOs: 139-1138; optionally wherein: (i) the amino acid sequence of (a), (b), (c), and/or (d) is present immediately subsequent to position 586, 587, 588, 589, 590, 591, 592, 593, 594, or 595, relative to a reference sequence numbered according to the amino acid sequence of SEQ ID NO: 138; or (ii) the amino acid sequence is not PLN, PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).

In yet another aspect, the present disclosure provides a polynucleotide encoding an AAV capsid polypeptide, e.g., an AAV capsid variant, wherein the AAV capsid variant comprises the amino acid sequence of any of SEQ ID NO: 1725-3622 or 3648-3659; an amino acid sequence comprising no more than four modifications, e.g., substitutions, relative to the amino acid sequence of any of SEQ ID NO: 1725-3622 or 3648-3659; or at least 3, 4, 5, 6, 7, 8, or 9 consecutive amino acids from the amino acid sequence of any of SEQ ID NO: 1725-3622 or 3648-3659. In some embodiments, the polynucleotide comprises the nucleotide sequence of any one of SEQ ID NOs: 4, 7, 3623-3635, or a nucleotide sequence with at least 80% (e.g., at least about 85, 90, 95, 96, 97, 98, or 99%) sequence identity thereto.

In yet another aspect, the present disclosure provides an AAV particle comprising an AAV capsid polypeptide, e.g., an AAV capsid variant, described herein. In some embodiments, the AAV particle comprises a nucleic acid sequence encoding a payload. In some embodiments, the AAV particle further comprises a viral genome comprising a promoter operably linked to the nucleic acid encoding the payload.

In yet another aspect, the present disclosure provides a method of making an AAV particle comprising an AAV capsid polypeptide, e.g., an AAV capsid variant, described herein. The method comprises providing a host cell comprising a viral genome and incubating the host cell under conditions suitable to enclose the viral genome in the AAV capsid variant, e.g., an AAV capsid variant described herein, thereby making the AAV particle.

In yet another aspect, the present disclosure provides a method of delivering a payload to a cell or tissue (e.g., a CNS cell, a CNS tissue, a muscle cell, or a muscle tissue). The method comprising administering an effective amount of an AAV particle comprising an AAV capsid variant described herein.

In yet another aspect, the present disclosure provides a method of treating a subject having or diagnosed with having a genetic disorder e.g., a monogenic disorder or a polygenic disorder. The method comprising administering an effective amount of an AAV particle comprising an AAV capsid variant described herein.

In yet another aspect, the present disclosure provides a method of treating a subject having or diagnosed with having a neurological, e.g., a neurodegenerative, disorder. The method comprising administering an effective amount of an AAV particle comprising an AAV capsid variant described herein.

In yet another aspect, the present disclosure provides a method of treating a subject having or diagnosed with having a muscular disorder or a neuromuscular disorder. The method comprising administering an effective amount of an AAV particle comprising an AAV capsid variant described herein.

In yet another aspect, the present disclosure provides a method of treating a subject having or diagnosed with having a cardiac disorder, e.g., a cardiac disorder as described herein (e.g., a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction). The method comprising administering an effective amount of an AAV particle comprising an AAV capsid variant described herein.

In yet another aspect, the present disclosure provides a method of treating a subject having or diagnosed with having a neuro-oncological disorder. The method comprising administering an effective amount of an AAV particle comprising an AAV capsid variant described herein.

In yet another aspect, the present disclosure provides a pharmaceutical formulation comprising (a) an AAV particle, or a variant thereof, e.g., an AAV particle comprising an AAV capsid variant described herein, (b) a buffering agent (e.g., Tris), (c) a polyether (e.g., glycerol), (d) a salt (e.g., sodium chloride), and (e) a surfactant (e.g., a poloxamer such as Pluronic F-68).

In yet another aspect, the present disclosure provides a pharmaceutical formulation comprising (a) an AAV particle, or a variant thereof, e.g., an AAV particle comprising an AAV capsid variant described herein, (b) a buffering agent (e.g., Tris), (c) a sugar (e.g., trehalose), (d) a salt (e.g., sodium chloride), and (e) a surfactant (e.g., a poloxamer such as Pluronic F-68).

In yet another aspect, the present disclosure provides a pharmaceutical formulation comprising: (a) an AAV particle, or a variant thereof, e.g., an AAV particle comprising an AAV capsid variant described herein, 20 mM or about 20 mM Tris, 1% or about 1% glycerol, 62.5 mM or about 62.5 mM sodium chloride, and 0.001% or about 0.001% Pluronic F-68; (b) an AAV particle, or a variant thereof, e.g., an AAV particle comprising an AAV capsid variant described herein, 20 mM or about 20 mM Tris, 2.5% or about 2.5% glycerol, 62.5 mM or about 62.5 mM sodium chloride, and 0.001% or about 0.001% Pluronic F-68; or (c) an AAV particle, or a variant thereof, e.g., an AAV particle comprising an AAV capsid variant described herein, 20 mM or about 20 mM Tris, 5.95% or about 5.95% trehalose, 62.5 mM or about 62.5 mM sodium chloride, and 0.001% or about 0.001% Pluronic F-68.

In some embodiments, the formulation has a pH of between 7.8-8.4 (e.g., 7.8, 7.9, 8.0, 8.1, 8.2, 8.3, or 8.4). In some embodiments, the formulation has an osmolality of between 250-650 mOsm/kg (e.g., between 250-600, 250-500, 250-450, 250-350, 300-550, 300-500, 300-450, 300-400, 350-400, 400-600, 400-550, 450-600, 450-550). In some embodiments, the formulation remains stable after storage at −80° C., 2-8° C., or room temperature for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), as reflected by a change (increase or decrease) in osmolality, viral titer, occupancy, and/or aggregation, relative to baseline (e.g., prior to storage) of less than 50% (e.g., less than 40%, 30%, 20%, 10%, or 5%).

In some embodiments, the AAV particle, or variant thereof, in the pharmaceutical formulation is an AAV particle, or variant thereof described herein (e.g., a TTD-001, TTD-002, TTD-003, TTD-004, TTD-005, TTD-006, TTD-007, TTD-008, TTD-009, TTD-010, TTD-011, TTD-012, TTD-013, or TTD-014 capsid variant, as described in Tables 3 and 4, or an AAV capsid variant comprising an amino acid sequence, e.g., as provided in any one of Tables 1A, 1B, 2-7, 10 11, or 20, or a variant thereof).

Those skilled in the art will recognize or be able to ascertain using no more than routine experimentation, many equivalents to the specific embodiments of the invention described herein. Such equivalents are intended to be encompassed by the following enumerated embodiments.

### ENUMERATED EMBODIMENTS

1. An AAV capsid variant, comprising an amino sequence comprising the following formula: [N1]-[N2], wherein:
   (i) [N1] comprises X1, X2, X3, X4, and X5, wherein:
      (a) position X1 is: P, Q, A, H, K, L, R, S, or T;
      (b) position X2 is: L, I, V, H, or R;
      (c) position X3 is: N, D, I, K, or Y;
      (d) position X4 is: G, A, C, R, or S; and
      (e) position X5 is: A, S, T, G, C, D, N, Q, V, or Y; and
   (ii) [N2] comprises the amino acid sequence of VHLY (SEQ ID NO: 4680), VHIY (SEQ ID NO: 4681), VHVY (SEQ ID NO: 4682), or VHHY (SEQ ID NO: 4683); and/or an amino acid modification, e.g., a conservative substitution, of any of the aforesaid amino acids in (i) and/or (ii);
   optionally wherein the AAV capsid variant further comprises:
   (a) one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138; and/or
   (b) one, two, or all of an amino acid other than V at position 596 (e.g., D, F, G, L, A, E, or I), an amino acid other than Q at position 597 (e.g., P, K, R, H, E, or L), and/or an amino acid other than N at position 598 (e.g., T, K, H, D, Y, S, I, or P), relative to a reference sequence numbered according to the amino acid sequence of SEQ ID NO: 138.
2. An AAV capsid variant comprising one, two, three, four, or all of:
   (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722);
   (ii) an [N2] wherein [N2] is or comprises: VHLY (SEQ ID NO: 4680), VHVY (SEQ ID NO: 4682), VPLY (SEQ ID NO: 4723), VNLY (SEQ ID NO: 4724), VHRY (SEQ ID NO: 4725), VHIY (SEQ ID NO: 4681), VHHY (SEQ ID NO: 4683), FHLY (SEQ ID NO: 4726), LHLY (SEQ ID NO: 4727), DHLY (SEQ ID NO: 4728), VQLY (SEQ ID NO: 4729), IHLY (SEQ ID NO: 4730), VDLY (SEQ ID NO: 4731), AHLY (SEQ ID NO: 4732), VLLY (SEQ ID NO: 4733), GHLY (SEQ ID NO: 4734), VRLY (SEQ ID NO: 4735), or VYLY (SEQ ID NO: 4736);
   (iii) an [N3] wherein [N3] is or comprises: AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751);
   (iv) an [N4] wherein [N4] is or comprises: TGW, LSP, TGL, TGS, TGG, TAW, TGR, TAS, LSS, TSS, SSL, SSS, TLS, TVS, VSS, TSP, VSP, TMS, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or
   (v) an [N5] wherein [N5] is or comprises: VQN, VPN, VKN, VQT, VQK, DQN, VQH, GQN, VQI, VHN, FQN, LQN, VLN, VRN, VQS, VQY, AQN, VEN, VQD, IQN, VKK, DKN, VKT, VQP, EQN, GQT, FQK, GHN, or VPH; and/or
   wherein the AAV capsid variant comprises an amino acid modification, e.g., a conservative substitution, of any of the aforesaid amino acids in (i)-(v).
3. The AAV capsid variant of embodiment 1, wherein:
   (a) position X1 is: P, Q, A, S, or T;
   (b) position X2 is: L or I;
   (c) position X3 is: N or D;
   (d) position X4 is: G or S; and/or
   (e) position X5 is: A, S, G, T, or N.
4. The AAV capsid variant of embodiment 1 or 3, wherein position X1 is P; position X2 is L; position X3 is N; position X4 is G or S; and/or position X5 is A.
5. The AAV capsid variant of embodiment 1, 3 or 4, wherein [N1] comprises PL, NG, AL, PI, QL, SL, TL, LN, LD, IN, DG, DS, GA, SA, SS, GG, GN, GS, or GT.
6. The AAV capsid variant of any one of embodiments 1 or 3-5, wherein [N1] comprises PLN, ALD, ALN, PIN, PLD, QLN, SLD, SLN, TLN, LNG, LDG, ING, LDS, NGA, DGA, DSA, DSS, NGG, NGN, NGS, NGT.
7. The AAV capsid variant of any one of embodiments 1 or 3-6, wherein [N1] comprises PLNG (SEQ ID NO: 3678), ALDG (SEQ ID NO: 4762), ALNG (SEQ ID NO: 4758), PING (SEQ ID NO: 4763), PLDG (SEQ ID NO: 4764), PLDS (SEQ ID NO: 4765), QLNG (SEQ ID NO: 4766), SLDG (SEQ ID NO: 4767), SLNG (SEQ ID NO: 4756), or TLNG (SEQ ID NO: 4754).
8. The AAV capsid variant of any one of embodiments embodiment 1-7, wherein [N1] is or comprises PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722).
9. The AAV capsid variant of any one of embodiments 1-8, wherein [N1] is or comprises ALDGA (SEQ ID NO: 4698), ALNGA (SEQ ID NO: 4686), PINGA (SEQ ID NO: 4697), PLDGA (SEQ ID NO: 4691), PLDSA (SEQ ID NO: 4701), PLDSS (SEQ ID NO: 4705), PLNGA (SEQ ID NO: 3679), PLNGG (SEQ ID NO: 4689), PLNGN (SEQ ID NO: 4693), PLNGS (SEQ ID NO: 4687), PLNGT (SEQ ID NO: 4690), QLNGA (SEQ ID NO: 4685), SLDGA (SEQ ID NO: 4694), SLNGA (SEQ ID NO: 4684), or TLNGA (SEQ ID NO: 4708).
10. The AAV capsid variant of any one of embodiments 1-9, wherein [N1] is or comprises PLNGA (SEQ ID NO: 3679).
11. The AAV capsid variant of any one of embodiment 1 or 3-10, wherein [N1]-[N2] comprises:
   (i) LDGAVHLY (SEQ ID NO: 4768), LNGAVHLY (SEQ ID NO: 4769), INGAVHLY (SEQ ID NO: 4770), LDSAVHLY (SEQ ID NO: 4771), LDSSVHLY (SEQ ID NO: 4772), LNGGVHLY (SEQ ID NO: 4773), LNGNVHLY (SEQ ID NO: 4774), LNGSVHLY (SEQ ID NO: 4775), LNGTVHLY (SEQ ID NO: 4776), LNGAVHIY (SEQ ID NO: 4777), LDGAVHVY (SEQ ID NO: 4778), or LNGAVHHY (SEQ ID NO: 4779);
   (ii) an amino acid sequence comprising any portion of an amino acid sequence in (i), e.g., any 2, 3, 4, 5, 6, or 7 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
12. The AAV capsid variant of any one of embodiments 1-11, wherein [N1]-[N2] is or comprises:
   (i) PLNGAVHLY (SEQ ID NO: 3648), ALDGAVHLY (SEQ ID NO: 4780), ALNGAVHLY (SEQ ID NO: 4781), PINGAVHLY (SEQ ID NO: 4782), PLDGAVHLY (SEQ ID NO: 4783), PLDSAVHLY (SEQ ID NO: 4784), PLDSSVHLY (SEQ ID NO: 4785), PLNGGVHLY (SEQ ID NO: 4786), PLNGNVHLY (SEQ ID NO: 4787), PLNGSVHLY (SEQ ID NO: 4788), PLNGTVHLY (SEQ ID NO: 4789), QLNGAVHLY (SEQ ID NO: 4790), SLDGAVHLY (SEQ ID NO: 4791), SLNGAVHLY (SEQ ID NO: 4792), TLNGAVHLY (SEQ ID NO: 4793), PLNGAVHIY (SEQ ID NO: 4794), PLDGAVHVY (SEQ ID NO: 4795), or PLNGAVHHY (SEQ ID NO: 4796);
   (ii) an amino acid sequence comprising any portion of an amino acid sequence in (i), e.g., any 2, 3, 4, 5, 6, 7, or 8 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
13. The AAV capsid variant of any one of embodiments 1-12, wherein [N1]-[N2] is or comprises PLNGAVHLY (SEQ ID NO: 3648).
14. The AAV capsid variant of any one of embodiments 1-13, which further comprises one, two, three, or all of an amino acid other than A at position 589 (e.g., D, S, or T), an amino acid other than Q at position 590 (e.g., K, H, L, P, or R), an amino acid other than A at position 591 (e.g., P, E, or R), and/or an amino acid other than Q at position 592 (e.g., H, K, or P), relative to a reference sequence numbered according to the amino acid sequence of SEQ ID NO: 138.
15. The AAV capsid variant of any one of embodiments 1-14, which further comprises one, two, three, or all of an amino acid other than A at position 596 (e.g., D, S, or T), an amino acid other than Q at position 597 (e.g., K, H, L, P, or R), an amino acid other than A at position 598 (e.g., P, E, or R), and/or an amino acid other than Q at position 599 (e.g., H, K, or P), numbered according to the amino acid sequence of SEQ ID NO: 5, 8, or 3636.
16. The AAV capsid variant of any one of embodiments 1-13, which further comprises:
   (i) A at position 589, Q at position 590, A at position 591, and/or Q at position 592, numbered according to the amino acid sequence of SEQ ID NO: 138; or
   (ii) A at position 596, Q at position 597, A at position 598, and/or Q at position 599, numbered according to the amino acid sequence of SEQ ID NO: 5, 8, or 3636.
17. The AAV capsid variant of embodiment 1 or 3-16, which further comprises [N3], wherein [N3] comprises X6, X7, X8, and X9, wherein:
   (a) position X6 is: A, D, S, or T;
   (b) position X7 is: Q, K, H, L, P, or R;
   (c) position X8 is: A, P, E, or R; and
   (d) position X9 is: Q, H, K, or P; and/or an amino acid modification, e.g., a conservative substitution, of any of the aforesaid amino acids in (a)-(d).
18. The AAV capsid variant of embodiment 17, wherein:
   (a) position X6 is: A, D, S;
   (b) position X7 is: Q or K;
   (c) position X8 is: A or P; and/or
   (d) position X9 is: Q.
19. The AAV capsid variant of embodiment 17 or 18, wherein [N3] comprises AQ, SQ, AK, DQ, PQ, QA, QP, or KA.
20. The AAV capsid variant of any one of embodiments 17-19, wherein [N3] comprises AQA, AQP, SQA, AKA, DQA, QAQ, QPQ, or KAQ.
21. The AAV capsid variant of any one of embodiments 2 or 17-20, wherein [N3] is or comprises AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751).
22. The AAV capsid variant of any one of embodiments 2 or 17-21, wherein [N3] is or comprises AQAQ (SEQ ID NO: 4737), AQPQ (SEQ ID NO: 4739), SQAQ (SEQ ID NO: 4738), AKAQ (SEQ ID NO: 4741), or DQAQ (SEQ ID NO: 4744).
23. The AAV capsid variant of any one of embodiments 2 or 17-22, wherein [N3] is or comprises AQAQ (SEQ ID NO: 4737).
24. The AAV capsid variant of any one of embodiments 2 or 17-23, wherein [N2]-[N3] is or comprises:
   (i) VHLYAQAQ (SEQ ID NO: 4797), VHLYAQPQ (SEQ ID NO: 4798), VHLYSQAQ (SEQ ID NO: 4799), VHLYAKAQ (SEQ ID NO: 4800), VHLYDQAQ (SEQ ID NO: 4801), VHIYAQAQ (SEQ ID NO: 4802), VHVYAQAQ (SEQ ID NO: 4803), or VHHYAQAQ (SEQ ID NO: 4804);
   (ii) an amino acid sequence comprising any portion of an amino acid sequence in (i), e.g., any 2, 3, 4, 5, 6, or 7 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
25. The AAV capsid variant of any one of embodiments 2 or 17-24, wherein [N2]-[N3] is or comprises VHLYAQAQ (SEQ ID NO: 4797).
26. The AAV capsid variant of any one of embodiments 17-25, wherein [N1]-[N2]-[N3] comprises:
   (i) ALDGAVHLYAQ (SEQ ID NO: 4805), ALNGAVHLYAQ (SEQ ID NO: 4806), PINGAVHLYAQ (SEQ ID NO: 4807), PLDGAVHLYAQ (SEQ ID NO: 4808), PLDGAVHLYSQ (SEQ ID NO: 4809), PLDSAVHLYAQ (SEQ ID NO: 4810), PLDSSVHLYAQ (SEQ ID NO: 4811), PLNGAVHLYAK (SEQ ID NO: 4812), PLNGAVHLYAQ (SEQ ID NO: 4813), PLNGAVHLYDQ (SEQ ID NO: 4814), PLNGAVHLYSQ (SEQ ID NO: 4815), PLNGGVHLYAQ (SEQ ID NO: 4816), PLNGNVHLYAQ (SEQ ID NO: 4817), PLNGSVHLYAQ (SEQ ID NO: 4818), PLNGTVHLYAQ (SEQ ID NO: 4819), QLNGAVHLYAQ (SEQ ID NO: 4820), SLDGAVHLYAQ (SEQ ID NO: 4821), SLNGAVHLYAQ (SEQ ID NO: 4822), TLNGAVHLYAQ (SEQ ID NO: 4823), PLNGAVHIYAQ (SEQ ID NO: 4824), PLDGAVHVYAQ (SEQ ID NO: 4825), or PLNGAVHHYAQ (SEQ ID NO: 4826);
   (ii) an amino acid sequence comprising any portion of an amino acid sequence in (i), e.g., any 2, 3, 4, 5, 6, 7, 8, 9, or 10 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
27. The AAV capsid variant of any one of embodiments 2 or 17-26, wherein [N1]-[N2]-[N3] is or comprises:
   (i) PLNGAVHLYAQAQ (SEQ ID NO: 4836), ALDGAVHLYAQAQ (SEQ ID NO: 4827), ALNGAVHLYAQAQ (SEQ ID NO: 4828), PINGAVHLYAQAQ (SEQ ID NO: 4829), PLDGAVHLYAQAQ (SEQ ID NO: 4830), PLDGAVHLYAQPQ (SEQ ID NO: 4831), PLDGAVHLYSQAQ (SEQ ID NO: 4832), PLDSAVHLYAQAQ (SEQ ID NO: 4833), PLDSSVHLYAQAQ (SEQ ID NO: 4834), PLNGAVHLYAKAQ (SEQ ID NO: 4835), PLNGAVHLYAQPQ (SEQ ID NO: 4837), PLNGAVHLYDQAQ (SEQ ID NO: 4838), PLNGAVHLYSQAQ (SEQ ID NO: 4839), PLNGGVHLYAQAQ (SEQ ID NO: 4840), PLNGNVHLYAQAQ (SEQ ID NO: 4841), PLNGSVHLYAQAQ (SEQ ID NO: 4842), PLNGTVHLYAQAQ (SEQ ID NO: 4843), QLNGAVHLYAQAQ (SEQ ID NO: 4844), SLDGAVHLYAQAQ (SEQ ID NO: 4845), SLNGAVHLYAQAQ (SEQ ID NO: 4846), TLNGAVHLYAQAQ (SEQ ID NO: 4847), PLNGAVHIYAQAQ (SEQ ID NO: 4848), PLDGAVHVYAQAQ (SEQ ID NO: 4849), or PLNGAVHHYAQAQ (SEQ ID NO: 4850);
   (ii) an amino acid sequence comprising any portion of an amino acid sequence in (i), e.g., any 2, 3, 4, 5, 6, 7, 8, 9, 10, 11, or 12 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
28. The AAV capsid variant of any one of embodiments 2 or 17-27, wherein [N1]-[N2]-[N3] comprises PLNGAVHLYAQAQ (SEQ ID NO: 4836).
29. The AAV capsid variant of any one of embodiments 1-28, which further comprises one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138.
30. The AAV capsid variant of any one of embodiments 1-28, which further comprises one, two, or all of an amino acid other than T at position 600 (e.g., V, L, R, S, A, C, I, K, M, N, P, or Q), an amino acid other than G at position 601 (e.g., S, A, T, M, V, Q, L, H, I, K, N, P, R, or Y), and/or an amino acid other than W at position 602 (e.g., S, P, G, A, Q, L, M, K, C, E, F, H, R, T, V, or Y), relative to a reference sequence numbered according to the amino acid sequence of SEQ ID NO: 5, 8, or 3636.
31. The AAV capsid variant of any one of embodiments 1-30, which further comprises one, two, or all of:
   (i) the amino acid V, L, R, S, A, C, I, K, M, N, P, or Q (e.g., L) at position 593 numbered according to SEQ ID NO: 138 or at position 600 numbered according to SEQ ID NO: 5, 8, or 3636);
   (ii) the amino acid S, A, T, M, V, Q, L, H, I, K, N, P, R, or Y (e.g., S) at position 594 numbered according to SEQ ID NO: 138, or at position 601 numbered according to SEQ ID NO: 5, 8, or 3636; and/or
   (iii) the amino acid S, P, G, A, Q, L, M, K, C, E, F, H, R, T, V, or Y (e.g. P) at position 595 numbered according to SEQ ID NO: 138 or at position 602 numbered according to SEQ ID NO: 5, 8, or 3636).
32. The AAV capsid variant of any one of embodiments 1-31, which further comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138, or at position 601 numbered according to SEQ ID NO: 5, 8, or 3636; and
   (iii) the amino acid P at position 595 numbered according to SEQ ID NO: 138 or at position 602 numbered according to SEQ ID NO: 5, 8, or 3636.
33. The AAV capsid variant of any one of embodiments 1 or 3-28, which further comprises:
   (i) T at position 593, G at position 594, and/or W at position 595, numbered according to SEQ ID NO: 138;
   (ii) T at position 600, G at position 601, and/or W at position 602, numbered according to SEQ ID NO: 5, 8, or 3636.
34. The AAV capsid variant of any one of embodiments 1 or 3-33, which further comprises [N4], wherein [N4] comprises X10, X11, and X12, wherein:
   (a) position X10 is: T, V, L, R, S, A, C, I, K, M, N, P, or Q;
   (b) position X11 is: G, S, A, T, M, V, Q, L, H, I, K, N, P, R, or Y; and
   (c) position X12 is: W, S, P, G, A, Q, L, M, K, C, E, F, H, R, T, V, or Y; and/or an amino acid modification, e.g., a conservative substitution, of any of the aforesaid amino acids in (a)-(c).
35. The AAV capsid variant of embodiment 34, wherein:
   (a) position X10 is: T, V, L, A, R, C, S, I, M, N, P, Q;
   (b) position X11 is: G, A, S, T, M, Q, V; and/or
   (c) position X12 is: P, S, W, G, A, Q, T, K, N, R, L, M, H, V, C, or E.
36. The AAV capsid variant of embodiment 34 or 25, wherein:
   (a) position X10 is: T or L;
   (b) position X11 is: G or S; and/or
   (c) position X12 is: W or P.
37. The AAV capsid variant of any one of embodiments 34-36, wherein [N4] comprises LS, TG, LA, LT, SA, SS, TL, TT, TS, TA, TV, VS, AA, AG, AS, AT, CS, CT, IA, IG, IL, IQ, IS, IT, LG, LH, LK, LM, LN, LQ, MA, NA, NM, NS, NT, NV, QA, RA, RG, RI, RL, RM, RN, RQ, RS, RT, RV, SG, SM, ST, SV, TK, TM, TN, TP, TQ, TR, VA, VG, VH, VK, VL, VM, VN, VQ, VR, VT, PG, LV, SP, GW, AP, GR, AL, AW, GG, GS, GP, QP, QS, AH, AN, AQ, AR, GQ, HP, KS, MG, MP, MQ, MS, NP, QQ, QR, SH, SK, SQ, SR, IP, VE, AK, AM, AV, GA, GC, GT, KA, KP, KQ, LP, MK, MN, MT, NQ, PP, QH, QK, QM, QN, QT, RW, SL, VW, GK, GN, NG, RP, SN, GL, or VP.
38. The AAV capsid variant of any one of embodiments 34-37, wherein [N4] is or comprises TGW, LSP, TGL, TGS, TGG, TAW, TGR, TAS, LSS, TSS, SSL, SSS, TLS, TVS, VSS, TSP, VSP, TMS, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW.
39. The AAV capsid variant of any one of embodiments 2 or 34-38, wherein [N4] is or comprises LSP, TGW, LAA, LTP, SAP, SSP, TGR, TLA, TTS, TSP, TAL, TAW, TGG, TGS, TVS, VSP, VSS, AAP AGP, ASP, ATP, CSP, CTP, IAA, IAG, IAS, IGG, IGS, ILG, IQP, IQS, ISG, ISP, ISS, ITP, LAG, LAH, LAN, LAP, LAQ, LAR, LAS, LAT, LGP, LGQ, LGS, LHP, LKS, LMA, LMG, LMP, LMQ, LMS, LNP, LQP, LQQ, LQR, LSH, LSK, LSQ, LSR, LST, LTA, LTN, LTS, MAP, NAQ, NAS, NMQ, NSP, NTP, NVQ, QAP, RAA, RAQ, RAS, RGG, RGS, RIA, RIG, RIP, RLG, RLS, RMS, RNS, RQP, RSA, RSG, RSP, RSQ, RSS, RST, RTA, RTG, RTL, RTS, RTT, RVE, SAA, SAK, SAM, SAQ, SGP, SMA, SMG, SMQ, SMS, STP, SVA, SVG, TAA, TAG, TAK, TAM, TAN, TAP, TAQ, TAS, TAT, TAV, TGA, TGC, TGP, TGT, TKA, TKP, TKQ, TKS, TLP, TLQ, TMA, TMG, TMK, TMN, TMP, TMQ, TMS, TMT, TNA, TNQ, TNS, TPP, TQH, TQK, TQM, TQN, TQP, TQQ, TQT, TRW, TSA, TSG, TSH, TSK, TSL, TSM, TSQ, TSS, TST, TSV, TTA, TTG, TTK, TTP, TTQ, TTT, TVA, TVG, TVQ, TVW, VAA, VAG, VAK, VAN, VAQ, VAS, VAT, VGG, VGK, VGN, VGS, VHP, VKA, VKP, VKQ, VLP, VLS, VMA, VMQ, VMS, VNA, VNG, VNS, VQA, VQN, VQP, VQQ, VQS, VQT, VRP, VSA, VSG, VSN, VSQ, VSR, VST, VTA, VTG, VTK, VTP, VTS, TGL, PGW, LSG, LSS, or LVP.
40. The AAV capsid variant of any one of embodiments 2 or 34-39, wherein [N4] is or comprises TGW.
41. The AAV capsid variant of any one of embodiments 2 or 34-39, wherein [N4] is or comprises LSP.
42. The AAV capsid variant of any one of embodiments 1-41, which further comprises one, two, or all of an amino acid other than V at position 596 (e.g., D, F, G, L, A, E, or I), an amino acid other than Q at position 597 (e.g., K, R, H, E, L, or P), and/or an amino acid other than N at position 598 (e.g., T, K, H, D, Y, S, I, or P), relative to a reference sequence numbered according to the amino acid sequence of SEQ ID NO: 138.
43. The AAV capsid variant of any one of embodiments 1-42, which further comprises one, two, or all of an amino acid other than V at position 603 (e.g., D, F, G, L, A, E, or I), an amino acid other than Q at position 604 (e.g., K, R, H, E, L, or P), and/or an amino acid other than N at position 605 (e.g., T, K, H, D, Y, S, I, or P), relative to a reference sequence numbered according to the amino acid sequence of SEQ ID NO: 5, 8, or 3636.
44. The AAV capsid variant of any one of embodiments 1-43, which further comprises one, two, or all of:
   (i) the amino acid V, D, F, G, L, A, E, or I at position 596 numbered according to SEQ ID NO: 138 or at position 603 numbered according to SEQ ID NO: 5, 8, or 3636;
   (ii) the amino acid K, R, H, E, L, or P at position 597 numbered according to SEQ ID NO: 138 or at position 604 numbered according to SEQ ID NO: 5, 8, or 3636; and/or
   (iii) the amino acid N, T, K, H, D, Y, S, I, or P at position 598 numbered according to the amino acid sequence of SEQ ID NO: 138 or at position 605 numbered according to SEQ ID NO: 5, 8, or 3636.
45. The AAV capsid variant of any one of embodiments 1-44, which further comprises one, two, or all of:
   (i) the amino acid V at position 596 numbered according to SEQ ID NO: 138 or at position 603 numbered according to SEQ ID NO: 5, 8, or 3636;
   (ii) the amino acid K, P, E or H at position 597 numbered according to SEQ ID NO: 138 or at position 604 numbered according to SEQ ID NO: 5, 8, or 3636; and
   (iii) the amino acid N at position 598 numbered according to the amino acid sequence of SEQ ID NO: 138 or at position 605 numbered according to SEQ ID NO: 5, 8, or 3636.
46. The AAV capsid variant of any one of embodiments 1 or 3-45, which further comprises [N5], wherein [N5] comprises X13, X14, and X15, wherein:
   (a) position X13 is: V, D, F, G, L, A, E, or I;
   (b) position X14 is: Q, K, R, H, E, L, or P; and
   (c) position X15 is: N, T, K, H, D, Y, S, I, or P; and/or an amino acid modification, e.g., a conservative substitution, of any of the aforesaid amino acids in (a)-(c).
47. The AAV capsid variant of embodiment 46, wherein:
   (a) position X13 is: V, D, A, F, E, G, or L;
   (b) position X14 is: Q, K, R, L, or P; and/or
   (c) position X15 is: N, T, K, H, D, I, K, S, or P.
48. The AAV capsid variant of embodiment 46 or 47, wherein position X14 is P.
49. The AAV capsid variant of embodiment 46 or 47, wherein position X14 is K.
50. The AAV capsid variant of embodiment 46 or 47, wherein position X14 is E or H.
51. The AAV capsid variant of embodiment 46 or 47, wherein position X14 is Q.
52. The AAV capsid variant of any one of embodiments 46-51, wherein [N5] comprises VQ, AQ, DQ, FQ, VL, LQ, EQ, GQ, VP, VR, VK, QN, QS, QT, QK, QH, LN, QI, PN, QD, QP, RN, or KN.
53. The AAV capsid variant of any one of embodiments 46-52, wherein [N5] is or comprises VQN, VPN, VKN, VQT, VQK, DQN, VQH, GQN, VQI, VHN, FQN, LQN, VLN, VRN, VQS, VQY, AQN, VEN, VQD, IQN, VKK, DKN, VKT, VQP, EQN, GQT, FQK, GHN, or VPH.
54. The AAV capsid variant of any one of embodiments 46-53, wherein [N5] is or comprises VQN, VKN AQN, VQS, DQN, VQT, VQK, VQH, FQN, VLN, LQN, VQI, EQN, GQT, VPN, VQD, VQP, or VRN.
55. The AAV capsid variant of any one of embodiments 46-54, wherein [N5] is or comprises VKN, VPN, VEN, or VHN.
56. The AAV capsid variant of any one of embodiments 46-54, wherein [N5] is or comprises VQN.
57. The AAV capsid variant of any one of embodiments 2 or 46-56, wherein [N4]-[N5] is or comprises:

| | (i) |
|---|---|
| (SEQ ID NO: 4851) | |
| | TGWVQN, |
| (SEQ ID NO: 5000) | |
| | TGWVPN, |
| (SEQ ID NO: 4852) | |
| | LAAVQN, |
| (SEQ ID NO: 4853) | |
| | LTPVQN, |
| (SEQ ID NO: 4854) | |
| | SAPVQN, |
| (SEQ ID NO: 4855) | |
| | SSPVQN, |
| (SEQ ID NO: 4856) | |
| | TGRVQN, |
| (SEQ ID NO: 4857) | |
| | TGWAQN, |
| (SEQ ID NO: 4858) | |
| | TGWVQS, |
| (SEQ ID NO: 4859) | |
| | TLAVQN, |
| (SEQ ID NO: 4860) | |
| | TTSVQN, |
| (SEQ ID NO: 4861) | |
| | TSPVQN, |
| (SEQ ID NO: 4862) | |
| | TALVQN, |
| (SEQ ID NO: 4863) | |
| | TAWVQN, |
| (SEQ ID NO: 4864) | |
| | TGGVQN, |
| (SEQ ID NO: 4865) | |
| | TGSVQN, |
| (SEQ ID NO: 4866) | |
| | TGWDQN, |
| (SEQ ID NO: 4867) | |
| | TVSVQN, |
| (SEQ ID NO: 4868) | |
| | VSPVQN, |
| (SEQ ID NO: 4869) | |
| | VSSVQN, |
| (SEQ ID NO: 4870) | |
| | AAPVQN, |
| (SEQ ID NO: 4871) | |
| | AGPVQN, |
| (SEQ ID NO: 4872) | |
| | ASPVQN, |
| (SEQ ID NO: 4873) | |
| | ATPVQN, |
| (SEQ ID NO: 4874) | |
| | CSPVQN, |
| (SEQ ID NO: 4875) | |
| | CTPVQN, |
| (SEQ ID NO: 4876) | |
| | IAAVQN, |
| (SEQ ID NO: 4877) | |
| | IAGVQN, |
| (SEQ ID NO: 4878) | |
| | IASVQN, |
| (SEQ ID NO: 4879) | |
| | IGGVQN, |
| (SEQ ID NO: 4880) | |
| | IGSVQN, |
| (SEQ ID NO: 4881) | |
| | ILGVQN, |
| (SEQ ID NO: 4882) | |
| | IQPVQN, |
| (SEQ ID NO: 4883) | |
| | IQSVQN, |
| (SEQ ID NO: 4884) | |
| | ISGVQN, |
| (SEQ ID NO: 4885) | |
| | ISPVQN, |
| (SEQ ID NO: 4886) | |
| | ISSVQN, |
| (SEQ ID NO: 4887) | |
| | ITPVQN, |
| (SEQ ID NO: 4888) | |
| | LAGVQN, |
| (SEQ ID NO: 4889) | |
| | LAHVQN, |
| (SEQ ID NO: 4890) | |
| | LANVQN, |
| (SEQ ID NO: 4891) | |
| | LAPVQN, |
| (SEQ ID NO: 4892) | |
| | LAPVQT, |
| (SEQ ID NO: 4893) | |
| | LAQVQN, |
| (SEQ ID NO: 4894) | |
| | LARVQN, |
| (SEQ ID NO: 4895) | |
| | LASVQN, |
| (SEQ ID NO: 4896) | |
| | LATVQN, |
| (SEQ ID NO: 4897) | |
| | LGPVQN, |
| (SEQ ID NO: 4898) | |
| | LGQVQN, |
| (SEQ ID NO: 4899) | |
| | LGSVQN, |
| (SEQ ID NO: 4900) | |
| | LHPVQN, |
| (SEQ ID NO: 4901) | |
| | LKSVQN, |
| (SEQ ID NO: 4902) | |
| | LMAVQN, |
| (SEQ ID NO: 4903) | |
| | LMGVQN, |
| (SEQ ID NO: 4904) | |
| | LMPVQN, |
| (SEQ ID NO: 4905) | |
| | LMQVQN, |
| (SEQ ID NO: 4906) | |
| | LMSVQN, |
| (SEQ ID NO: 4907) | |
| | LNPVQN, |
| (SEQ ID NO: 4908) | |
| | LQPVQN, |
| (SEQ ID NO: 4909) | |
| | LQQVQN, |
| (SEQ ID NO: 4910) | |
| | LQRVQN, |
| (SEQ ID NO: 4911) | |
| | LSHVQN, |
| (SEQ ID NO: 4912) | |
| | LSKVQN, |
| (SEQ ID NO: 4913) | |
| | LSPVQK, |
| (SEQ ID NO: 4914) | |
| | LSPVQN, |
| (SEQ ID NO: 4915) | |
| | LSQVQN, |
| (SEQ ID NO: 4916) | |
| | LSRVQN, |
| (SEQ ID NO: 4917) | |
| | LSTVQN, |
| (SEQ ID NO: 4918) | |
| | LTAVQN, |
| (SEQ ID NO: 4919) | |
| | LTNVQN, |
| (SEQ ID NO: 4920) | |
| | LTSVQN, |
| (SEQ ID NO: 4921) | |
| | MAPVQN, |
| (SEQ ID NO: 4922) | |
| | NAQVQN, |
| (SEQ ID NO: 4923) | |
| | NASVQN, |
| (SEQ ID NO: 4924) | |
| | NMQVQN, |
| (SEQ ID NO: 4925) | |
| | NSPVQN, |
| (SEQ ID NO: 4926) | |
| | NTPVQN, |
| (SEQ ID NO: 4927) | |
| | NVQVQN, |
| (SEQ ID NO: 4928) | |
| | QAPVQN, |
| (SEQ ID NO: 4929) | |
| | RAAVQN, |
| (SEQ ID NO: 4930) | |
| | RAQVQN, |
| (SEQ ID NO: 4931) | |
| | RASVQN, |
| (SEQ ID NO: 4932) | |
| | RGGVQN, |
| (SEQ ID NO: 4933) | |
| | RGSVQN, |
| (SEQ ID NO: 4934) | |
| | RIAVQN, |
| (SEQ ID NO: 4935) | |
| | RIGVQN, |
| (SEQ ID NO: 4936) | |
| | RIPVQN, |
| (SEQ ID NO: 4937) | |
| | RLGVQN, |
| (SEQ ID NO: 4938) | |
| | RLSVQN, |
| (SEQ ID NO: 4939) | |
| | RMSVQN, |
| (SEQ ID NO: 4940) | |
| | RNSVQN, |
| (SEQ ID NO: 4941) | |
| | RQPVQN, |
| (SEQ ID NO: 4942) | |
| | RSAVQN, |
| (SEQ ID NO: 4943) | |
| | RSGVQN, |
| (SEQ ID NO: 4944) | |
| | RSPVQN, |
| (SEQ ID NO: 4945) | |
| | RSQVQN, |
| (SEQ ID NO: 4946) | |
| | RSSVQN, |
| (SEQ ID NO: 4947) | |
| | RSTVQN, |
| (SEQ ID NO: 4948) | |
| | RTAVQN, |
| (SEQ ID NO: 4949) | |
| | RTGVQN, |
| (SEQ ID NO: 4950) | |
| | RTLVQN, |
| (SEQ ID NO: 4951) | |
| | RTSVQN, |
| (SEQ ID NO: 4952) | |
| | RTTVQN, |
| (SEQ ID NO: 4953) | |
| | RVEVQN, |
| (SEQ ID NO: 4954) | |
| | SAAVQN, |
| (SEQ ID NO: 4955) | |
| | SAKVQN, |
| (SEQ ID NO: 4956) | |
| | SAMVQN, |
| (SEQ ID NO: 4957) | |
| | SAQVQN, |
| (SEQ ID NO: 4958) | |
| | SGPVQN, |
| (SEQ ID NO: 4959) | |
| | SMAVQN, |
| (SEQ ID NO: 4960) | |
| | SMGVQN, |
| (SEQ ID NO: 4961) | |
| | SMQVQN, |
| (SEQ ID NO: 4962) | |
| | SMSVQN, |
| (SEQ ID NO: 4963) | |
| | STPVQN, |
| (SEQ ID NO: 4964) | |
| | SVAVQN, |
| (SEQ ID NO: 4965) | |
| | SVGVQN, |
| (SEQ ID NO: 4966) | |
| | TAAVQN, |
| (SEQ ID NO: 4967) | |
| | TAGVQN, |
| (SEQ ID NO: 4968) | |
| | TAKVQN, |
| (SEQ ID NO: 4969) | |
| | TAMVQN, |
| (SEQ ID NO: 4970) | |
| | TANVQN, |
| (SEQ ID NO: 4971) | |
| | TAPVQN, |
| (SEQ ID NO: 4972) | |
| | TAPVQT, |
| (SEQ ID NO: 4973) | |
| | TAQVQN, |
| (SEQ ID NO: 4974) | |
| | TASVQN, |
| (SEQ ID NO: 4975) | |
| | TASVQT, |
| (SEQ ID NO: 4976) | |
| | TATVQN, |
| (SEQ ID NO: 4977) | |
| | TAVVQN, |
| (SEQ ID NO: 4978) | |
| | TAWDQN, |
| (SEQ ID NO: 4979) | |
| | TAWVQH, |
| (SEQ ID NO: 4980) | |
| | TAWVQT, |
| (SEQ ID NO: 4981) | |
| | TGAVQN, |
| (SEQ ID NO: 4982) | |
| | TGCFQN, |
| (SEQ ID NO: 4983) | |
| | TGGAQN, |
| (SEQ ID NO: 4984) | |
| | TGGFQN, |
| (SEQ ID NO: 4985) | |
| | TGGVLN, |
| (SEQ ID NO: 4986) | |
| | TGGVQH, |
| (SEQ ID NO: 4987) | |
| | TGGVQK, |
| (SEQ ID NO: 4988) | |
| | TGGVQT, |
| (SEQ ID NO: 4989) | |
| | TGPVQN, |
| (SEQ ID NO: 4990) | |
| | TGSAQN, |
| (SEQ ID NO: 4991) | |
| | TGSLQN, |
| (SEQ ID NO: 4992) | |
| | TGSVQH, |
| (SEQ ID NO: 4993) | |
| | TGSVQI, |
| (SEQ ID NO: 4994) | |
| | TGSVQS, |
| (SEQ ID NO: 4995) | |
| | TGSVQT, |
| (SEQ ID NO: 4996) | |
| | TGTVQN, |
| (SEQ ID NO: 4997) | |
| | TGWEQN, |
| (SEQ ID NO: 4998) | |
| | TGWFQN, |
| (SEQ ID NO: 4999) | |
| | TGWGQT, |
| (SEQ ID NO: 5001) | |
| | TGWVQD, |
| (SEQ ID NO: 5002) | |
| | TGWVQP, |
| (SEQ ID NO: 5003) | |
| | TGWVQT, |
| (SEQ ID NO: 5004) | |
| | TGWVRN, |
| (SEQ ID NO: 5005) | |
| | TKAVQN, |
| (SEQ ID NO: 5006) | |
| | TKPVQN, |
| (SEQ ID NO: 5007) | |
| | TKQVQN, |
| (SEQ ID NO: 5008) | |
| | TKSVQN, |
| (SEQ ID NO: 5009) | |
| | TLPVQN, |
| (SEQ ID NO: 5010) | |
| | TLQVQN, |
| (SEQ ID NO: 5011) | |
| | TMAVQN, |
| (SEQ ID NO: 5012) | |
| | TMGVQN, |
| (SEQ ID NO: 5013) | |
| | TMKVQN, |
| (SEQ ID NO: 5014) | |
| | TMNVQN, |
| (SEQ ID NO: 5015) | |
| | TMPVQN, |
| (SEQ ID NO: 5016) | |
| | TMQVQN, |
| (SEQ ID NO: 5017) | |
| | TMSVKN, |
| (SEQ ID NO: 5018) | |
| | TMSVQN, |
| (SEQ ID NO: 5019) | |
| | TMSVQT, |
| (SEQ ID NO: 5020) | |
| | TMTVQN, |
| (SEQ ID NO: 5021) | |
| | TNAVQN, |
| (SEQ ID NO: 5022) | |
| | TNQVQN, |
| (SEQ ID NO: 5023) | |
| | TNSVQN, |
| (SEQ ID NO: 5024) | |
| | TPPVQN, |
| (SEQ ID NO: 5025) | |
| | TQHVQN, |
| (SEQ ID NO: 5026) | |
| | TQKVQN, |
| (SEQ ID NO: 5027) | |
| | TQMVQN, |
| (SEQ ID NO: 5028) | |
| | TQNVQN, |
| (SEQ ID NO: 5029) | |
| | TQPVQN, |
| (SEQ ID NO: 5030) | |
| | TQQVQN, |
| (SEQ ID NO: 5031) | |
| | TQTVQN, |
| (SEQ ID NO: 5032) | |
| | TRWDQN, |
| (SEQ ID NO: 5033) | |
| | TSAVQN, |
| (SEQ ID NO: 5034) | |
| | TSGVQN, |
| (SEQ ID NO: 5035) | |
| | TSHVQN, |
| (SEQ ID NO: 5036) | |
| | TSKVQN, |
| (SEQ ID NO: 5037) | |
| | TSLVQN, |
| (SEQ ID NO: 5038) | |
| | TSMVQN, |
| (SEQ ID NO: 5039) | |
| | TSPDQN, |
| (SEQ ID NO: 5040) | |
| | TSQVQN, |
| (SEQ ID NO: 5041) | |
| | TSSVQN, |
| (SEQ ID NO: 5042) | |
| | TSSVQT, |
| (SEQ ID NO: 5043) | |
| | TSTVQN, |
| (SEQ ID NO: 5044) | |
| | TSVVQN, |
| (SEQ ID NO: 5045) | |
| | TTAVQN, |
| (SEQ ID NO: 5046) | |
| | TTGVQN, |
| (SEQ ID NO: 5047) | |
| | TTKVQN, |
| (SEQ ID NO: 5048) | |
| | TTPVQN, |
| (SEQ ID NO: 5049) | |
| | TTPVQT, |
| (SEQ ID NO: 5050) | |
| | TTQVQN, |
| (SEQ ID NO: 5051) | |
| | TTTVQN, |
| (SEQ ID NO: 5052) | |
| | TVAVQN, |
| (SEQ ID NO: 5053) | |
| | TVAVQT, |
| (SEQ ID NO: 5054) | |
| | TVGVQN, |
| (SEQ ID NO: 5055) | |
| | TVQVQN, |
| (SEQ ID NO: 5056) | |
| | TVSVKN, |
| (SEQ ID NO: 5057) | |
| | TVWVQK, |
| (SEQ ID NO: 5058) | |
| | VAAVQN, |
| (SEQ ID NO: 5059) | |
| | VAGVQN, |
| (SEQ ID NO: 5060) | |
| | VAKVQN, |
| (SEQ ID NO: 5061) | |
| | VANVQN, |
| (SEQ ID NO: 5062) | |
| | VAQVQN, |
| (SEQ ID NO: 5063) | |
| | VASVQN, |
| (SEQ ID NO: 5064) | |
| | VATVQN, |
| (SEQ ID NO: 5065) | |
| | VGGVQN, |
| (SEQ ID NO: 5066) | |
| | VGKVQN, |
| (SEQ ID NO: 5067) | |
| | VGNVQN, |
| (SEQ ID NO: 5068) | |
| | VGSVQN, |
| (SEQ ID NO: 5069) | |
| | VHPVQN, |
| (SEQ ID NO: 5070) | |
| | VKAVQN, |
| (SEQ ID NO: 5071) | |
| | VKPVQN, |
| (SEQ ID NO: 5072) | |
| | VKQVQN, |
| (SEQ ID NO: 5073) | |
| | VLPVQN, |
| (SEQ ID NO: 5074) | |
| | VLSVQN, |
| (SEQ ID NO: 5075) | |
| | VMAVQN, |
| (SEQ ID NO: 5076) | |
| | VMQVQN, |
| (SEQ ID NO: 5077) | |
| | VMSVQN, |
| (SEQ ID NO: 5078) | |
| | VNAVQN, |
| (SEQ ID NO: 5079) | |
| | VNGVQN, |
| (SEQ ID NO: 5080) | |
| | VNSVQN, |
| (SEQ ID NO: 5081) | |
| | VQAVQN, |
| (SEQ ID NO: 5082) | |
| | VQNVQN, |
| (SEQ ID NO: 5083) | |
| | VQPVQN, |
| (SEQ ID NO: 5084) | |
| | VQQVQN, |
| (SEQ ID NO: 5085) | |
| | VQSVQN, |
| (SEQ ID NO: 5086) | |
| | VQTVQN, |
| (SEQ ID NO: 5087) | |
| | VRPVQN, |
| (SEQ ID NO: 5088) | |
| | VSAVQN, |
| (SEQ ID NO: 5089) | |
| | VSGVQN, |
| (SEQ ID NO: 5090) | |
| | VSNVQN, |
| (SEQ ID NO: 5091) | |
| | VSPVQT, |
| (SEQ ID NO: 5092) | |
| | VSQVQN, |
| (SEQ ID NO: 5093) | |
| | VSRVQN, |
| (SEQ ID NO: 5094) | |
| | VSSVQK, |
| (SEQ ID NO: 5095) | |
| | VSSVQT, |
| (SEQ ID NO: 5096) | |
| | VSTVQN, |
| (SEQ ID NO: 5097) | |
| | VTAVQN, |
| (SEQ ID NO: 5098) | |
| | VTGVQN, |
| (SEQ ID NO: 5099) | |
| | VTKVQN, |
| (SEQ ID NO: 5100) | |
| | VTPVQN, |
| (SEQ ID NO: 5101) | |
| | VTSVQN, |
| (SEQ ID NO: 5102) | |
| | TGLVQN, |
| (SEQ ID NO: 5103) | |
| | TGWVKN, |
| (SEQ ID NO: 5104) | |
| | PGWVQN, |
| (SEQ ID NO: 5105) | |
| | TGWVQH, |
| (SEQ ID NO: 5106) | |
| | LSGVQN, |
| (SEQ ID NO: 5107) | |
| | LSSVQN. |
| | or |
| (SEQ ID NO: 5108) | |
| | LVPVQN; |

(ii) an amino acid sequence comprising any portion of an amino acid sequence in (i), e.g., any 2, 3, 4, or 5 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
58. The AAV capsid variant of any one of embodiments 2 or 46-57, wherein [N4]-[N5] is or comprises TGWVQN (SEQ ID NO: 4851), LSPVKN (SEQ ID NO: 5109), or TGWVPN (SEQ ID NO: 5000).
59. The AAV capsid variant of embodiment 2 or 46-58, wherein:
   (i) [N1] is or comprises: PLNGA (SEQ ID NO: 3679), QLNGA (SEQ ID NO: 4685), PLDGA (SEQ ID NO: 4691), PLDSS (SEQ ID NO: 4705), ALNGA (SEQ ID NO: 4686), PLNGS (SEQ ID NO: 4687), PLNGG (SEQ ID NO: 4689), SLNGA (SEQ ID NO: 4684), PLNGN (SEQ ID NO: 4693), PLNGT (SEQ ID NO: 4690), ALDGA (SEQ ID NO: 4698), PLDSA (SEQ ID NO: 4701), SLDGA (SEQ ID NO: 4694), TLNGA (SEQ ID NO: 4708), or PINGA (SEQ ID NO: 4697);
   (ii) [N2] is or comprises: VHLY (SEQ ID NO: 4680), VHHY (SEQ ID NO: 4683), VHVY (SEQ ID NO: 4682), or VHIY (SEQ ID NO: 4681);
   (iii) [N3] is or comprises: AQAQ (SEQ ID NO: 4737), AQPQ (SEQ ID NO: 4739), AKAQ (SEQ ID NO: 4741), DQAQ (SEQ ID NO: 4744), or SQAQ (SEQ ID NO: 4738);
   (iv) [N4] is or comprises: LSP, TGW, TMS, TTK, TGS, TTS, TSP, TMK, VAQ, TGG, TAW, VKQ, SAP, LSK, LAP, LAQ, VAS, TAK, SAK, TGC, TQK, TGR, TVA, SSP, TTQ, TAQ, RIA, RAS, TTP, LAS, LTP, STP, VSQ, TMQ, TSK, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, AGP, LAR, TTT, TLQ, VAK, RAA, TVG, LNP, LSQ, TKP, TNA, LAT, VTP, VQA, CTP, TAG, TSQ, TMN, TST, VKP, ASP, VAA, LKS, IAA, TAA, TKA, VSN, TAP, LMP, LHP, RAQ, LTN, RTT, TSV, TLA, RMS, VGN, LMQ, TAT, VHP, ISS, TRW, TMT, RSS, PGW, RTG, VAT, VTS, VSS, TSS, TNS, VKA, SGP, TGP, TAM, TQP, TQQ, VSR, VLP, LGS, VSA, VLS, TQH, QAP, NAQ, ATP, VQP, TTA, LAA, RSG, LMA, TMP, LAN, VST, SAQ, NTP, TGL, TLP, TAV, RLG, RTL, TQM, ITP, TVW, RSA, TAS, TMG, VQS, ISP, VGG, TAL, LAG, RTA, RSP, LAH, TSL, RLS, LMG, SMQ, TQT, VGS, VSG, VMA, IGG, IAG, LSH, VQT, RNS, TKQ, LGQ, NMQ, NVQ, RGG, VMS, TTG, LSR, MAP, ILG, TGT, TSH, RIG, SAM, TSM, SMG, SMS, TSG, TGA, VNS, VAG, IGS, VNG, LSS, LTA, VQN, TKS, SVG, NAS, TSA, TAN, LTS, RSQ, RIP, LVP, RVE, SVA, LSG, LQQ, LST, SAA, RTS, TQN, VNA, or LMS; and/or (v) [N5] is or comprises: VQN, VPN, DQN, VQH, FQN, VQD, VQS, VQT, VRN, AQN, VQP, VKN, VQK, EQN, VQI, LQN, GQT, or VLN.
60. The AAV capsid variant of any one of embodiments 1, 2, 8, 14-17, 21, 25, 28, 29-34, 38, 40-46, 48-51, or 53, wherein the amino acid sequence comprises:
   (i) the amino acid sequence of any of SEQ ID NOs: 139-1138;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
61. The AAV capsid variant of any one of embodiments 1-60, wherein the amino acid sequence comprises:
   (i) the amino acid sequence of any of SEQ ID NOs: 139-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
62. An AAV capsid variant comprising one, two, three, four, or all of:
   (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), or PLDSS (SEQ ID NO: 4705);
   (ii) an [N2] wherein [N2] is or comprises: VHLY (SEQ ID NO: 4680) or VHVY (SEQ ID NO: 4682);
   (iii) an [N3] wherein [N3] is or comprises: AQAQ (SEQ ID NO: 4737), SQAQ (SEQ ID NO: 4738), AQPQ (SEQ ID NO: 4739), or AQSQ (SEQ ID NO: 4740);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or
   (v) an [N5] wherein [N5] is or comprises: VQN, VKN, VQT, VQK, DQN, VQH, GQN, VQI, VHN, FQN, LQN, VLN, VRN, VQS, VQY, AQN, VEN, or VQD; and/or wherein the AAV capsid variant comprises an amino acid modification, e.g., a conservative substitution, of any of the aforesaid amino acids in (i)-(v).
63. The AAV capsid variant of embodiment 2 or 62, wherein:
   (i) [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), or PLDSS (SEQ ID NO: 4705);
   (ii) [N2] is or comprises: VHLY (SEQ ID NO: 4680) or VHVY (SEQ ID NO: 4682);
   (iii) [N3] is or comprises: AQAQ (SEQ ID NO: 4737), SQAQ (SEQ ID NO: 4738), AQPQ (SEQ ID NO: 4739), or AQSQ (SEQ ID NO: 4740);
   (iv) [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or
   (v) [N5] is or comprises: VQN, VKN, VQT, VQK, DQN, VQH, GQN, VQI, VHN, FQN, LQN, VLN, VRN, VQS, VQY, AQN, VEN, or VQD.
64. The AAV capsid variant of any one of embodiments 1, 2, 9, 19, 42, or 63, wherein the amino acid sequence comprises:
   (i) the amino acid sequence of any of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
65. The AAV capsid variant of any one of embodiments 1-64, wherein [N1]-[N2]-[N3]-[N4]-[N5] is or comprises the amino acid sequence of PLNGAVHLYAQAQTGWVPN (SEQ ID NO: 314).
66. The AAV capsid variant of any one of embodiments 1-64, wherein [N1]-[N2]-[N3]-[N4]-[N5] is or comprises the amino acid sequence of PLNGAVHLYAQAQLSPVKN (SEQ ID NO: 566).
67. The AAV capsid variant of any one of embodiments 1-64, wherein [N1]-[N2]-[N3]-[N4]-[N5] is or comprises the amino acid sequence of PLNGAVHLYAQAQTGWVQN (SEQ ID NO: 476).
68. The AAV capsid variant of any one of embodiments 1-64, wherein [N1]-[N2]-[N3]-[N4]-[N5] is or comprises the amino acid sequence of:
   (i) the amino acid sequence of any of SEQ ID NOs: 14-17, 40-136, 314, 325, 491, 499, 529, 558, 566, 576, 603, 610, 625, 631, 648, 649, 700, 703, 720, 755, 763, 765, 771, 791, 804, 816, 818, 819, 828, 859, 864, 871, 885, 946, 960, 966, 978, 979, 1016, 1033, 1032, 1037, 1058, 1081, 1100, 1122, or 1174-1193;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
69. The AAV capsid variant of any one of embodiments 1-68, wherein [N1]-[N2] is present in loop VIII.
70. The AAV capsid variant of any one of embodiments 2 or 46-69, wherein [N3], [N4], and/or [N5] is present in loop VIII.
71. The AAV capsid variant of any one of embodiments 2 or 46-70, wherein [N1]-[N2]-[N3]-[N4]-[N5] is present in loop VIII.
72. The AAV capsid variant of any one of embodiments 1-71, which comprises an amino acid other than A at position 587 and/or an amino acid other than Q at position 588, numbered according to SEQ ID NO: 138.
73. The AAV capsid variant of any one of embodiments 1-72, which comprises:
   (i) the amino acid P, Q, A, H, K, L, R, S, or T (e.g., P, Q, A, S, or T) at position 587, numbered according to SEQ ID NO: 138 or 3636; and/or
   (ii) the amino acid L, I, V, H, or R (e.g., L or I) at position 588, numbered according to SEQ ID NO: 5, 8, 138 or 3636.
74. The AAV capsid variant of any one of embodiments 1-73, wherein [N1] is present immediately subsequent to position 586, numbered according to the amino acid sequence of SEQ ID NO: 5, 8, 138, or 3636.
75. The AAV capsid variant of any one of embodiments 1-74, wherein [N1] is present immediately subsequent to position 586, and replaces positions 587 and 588 (e.g., A587 and Q588), numbered according to SEQ ID NO: 138.
76. The AAV capsid variant of any one of embodiments 1-75, wherein [N1] replaces positions 587 and 588 (e.g., A587 and Q588), numbered according to SEQ ID NO: 138.
77. The AAV capsid variant of any one of embodiments 1-76, wherein [N1] corresponds to positions 587-591 of SEQ ID NO: 5, 8, or 3636.
78. The AAV capsid variant of any one of embodiments 2 or 46-77, wherein [N1]-[N2]-[N3]-[N4]-[N5] is present immediately subsequent to position 586, numbered according to the amino acid sequence of SEQ ID NO: 5, 8, 138, or 3636.
79. The AAV capsid variant of any one of embodiments 1-78, wherein [N2] is present immediately subsequent to [N1].
80. The AAV capsid variant of any one of embodiments 1-79, wherein [N2] is present immediately subsequent to [N1], wherein [N1] is present immediately subsequent to position 586, relative to a reference sequence numbered according to the amino acid sequence of SEQ ID NO: 5, 8, 138, or 3636.
81. The AAV capsid variant of any one of embodiments 1-80, wherein [N2] is present immediately subsequent to [N1], wherein [N1] is present immediately subsequent to position 586 and replaces positions 587 and 588 (e.g., A587 and Q588), numbered according to SEQ ID NO: 138.
82. The AAV capsid variant of any one of embodiments 1-81, wherein [N2] corresponds to positions 592 to 595 of SEQ ID NO: 5, 8, or 3636.
83. The AAV capsid variant of any one of embodiments 1-82, wherein [N1]-[N2] replaces positions 587 and 588 (e.g., A587 and Q588), numbered according to SEQ ID NO: 138.
84. The AAV capsid variant of any one of embodiments 1-83, wherein [N1]-[N2] is present immediately subsequent to position 586, numbered according to SEQ ID NO: 138.
85. The AAV capsid variant of any one of embodiments 1-84, wherein [N1]-[N2] is present immediately subsequent to position 586 and replaces positions 587 and 588 (e.g., A587 and Q588), numbered according to SEQ ID NO: 138.
86. The AAV capsid variant of any one of embodiments 1-85, wherein [N1]-[N2] corresponds to positions 587-595 of SEQ ID NO: 5, 8, or 3636.
87. The AAV capsid variant of any one of embodiments 2 or 14-86, wherein [N3] is present immediately subsequent to position 588, relative to a reference sequence numbered according to the amino acid sequence of SEQ ID NO: 138.
88. The AAV capsid variant of any one of embodiments 2 or 14-87, wherein [N3] replaces positions 589-592 (e.g., A589, Q590, A591, Q592), numbered according to the amino acid sequence of SEQ ID NO: 138.
89. The AAV capsid variant of any one of embodiments 2 or 14-87, wherein [N3] is present immediately subsequent to position 588, and replaces positions 589-592 (e.g., A589, Q590, A591, Q592), numbered according to the amino acid sequence of SEQ ID NO: 138.
90. The AAV capsid variant of any one of embodiments 2 or 14-89, wherein [N3] corresponds to positions 596-599 of SEQ ID NO: 5, 8, or 3636.
91. The AAV capsid variant of any one of embodiments 2 or 14-90, wherein [N1]-[N2]-[N3] is present immediately subsequent to position 586, numbered according to SEQ ID NO: 138.
92. The AAV capsid variant of any one of embodiments 2 or 14-91, wherein [N1]-[N2]-[N3] replaces positions 587-592 (e.g., A587, Q588, A589, Q590, A591, Q592), numbered according to SEQ ID NO: 138.
93. The AAV capsid variant of any one of embodiments 2 or 14-92, wherein [N1]-[N2]-[N3] is present immediately subsequent to position 586 and replaces positions 587-592 (e.g., A587, Q588, A589, Q590, A591, Q592), numbered according to SEQ ID NO: 138.
94. The AAV capsid variant of any one of embodiments 2 or 14-93, wherein [N1]-[N2]-[N3] corresponds to positions 587-599 of SEQ ID NO: 5, 8, or 3636.
95. The AAV capsid variant of any one of embodiments 2 or 29-94, wherein [N4] is present immediately subsequent to position 592, relative to a reference sequence numbered according to the amino acid sequence of SEQ ID NO: 138.
96. The AAV capsid variant of any one of embodiments 2 or 29-95, wherein [N4] replaces positions 593-595 (e.g., T593, G594, W595), numbered according to the amino acid sequence of SEQ ID NO: 138.
97. The AAV capsid variant of any one of embodiments 2 or 29-96, wherein [N4] is present immediately subsequent to position 592, and replaces positions 593-595 (e.g., T593, G594, W595), numbered according to the amino acid sequence of SEQ ID NO: 138.
98. The AAV capsid variant of any one of embodiments 2 or 29-97, wherein [N4] corresponds to positions 600-602 of SEQ ID NO: 5, 8, or 3636.
99. The AAV capsid variant of any one of embodiments 2 or 29-97, wherein [N3]-[N4] is present immediately subsequent to position 588, numbered according to SEQ ID NO: 138.
100. The AAV capsid variant of any one of embodiments 2 or 29-97, wherein [N3]-[N4] replaces positions 589-595 (e.g., A589, Q590, A591, Q592, T593, G594, W595), numbered according to the amino acid sequence of SEQ ID NO: 138.
101. The AAV capsid variant of any one of embodiments 2 or 29-100, wherein [N3]-[N4] is present immediately subsequent to 588, and replaces positions 589-595 (e.g., A589, Q590, A591, Q592, T593, G594, W595), numbered according to the amino acid sequence of SEQ ID NO: 138.
102. The AAV capsid variant of any one of embodiments 2 or 29-101, wherein [N3]-[N4] corresponds to positions 596-602 of SEQ ID NO: 5, 8, or 3636.
103. The AAV capsid variant of any one of embodiments 2 or 29-102, wherein [N1]-[N2]-[N3]-[N4] is present immediately subsequent to position 586, numbered according to SEQ ID NO: 138.
104. The AAV capsid variant of any one of embodiments 2 or 29-103, wherein [N1]-[N2]-[N3]-[N4] replaces positions 587-595 (e.g., A587, Q588, A589, Q590, A591, Q592, T593, G594, W595), numbered according to SEQ ID NO: 138.
105. The AAV capsid variant of any one of embodiments 2 or 29-104, wherein [N1]-[N2]-[N3]-[N4] is present immediately subsequent to position 586 and replaces positions 587-595 (e.g., A587, Q588, A589, Q590, A591, Q592, T593, G594, W595), numbered according to SEQ ID NO: 138.
106. The AAV capsid variant of any one of embodiments 2 or 29-105, wherein [N1]-[N2]-[N3]-[N4] corresponds to positions 587-602 of SEQ ID NO: 5, 8, or 3636.
107. The AAV capsid variant of any one of embodiments 2 or 42-106, wherein [N5] is present immediately subsequent to position 595, relative to a reference sequence numbered according to the amino acid sequence of SEQ ID NO: 138.
108. The AAV capsid variant of any one of embodiments 2 or 42-107, wherein [N5] replaces positions 596-598 (e.g., V596, Q597, N598), numbered according to the amino acid sequence of SEQ ID NO: 138.
109. The AAV capsid variant of any one of embodiments 2 or 42-108, wherein [N5] is present immediately subsequent to position 595, and replaces positions 596-598 (e.g., V596, Q597, N598), numbered according to the amino acid sequence of SEQ ID NO: 138.
110. The AAV capsid variant of any one of embodiments 2 or 42-109, wherein [N5] corresponds to positions 603-605 of SEQ ID NO: 5, 8, or 3636.
111. The AAV capsid variant of any one of embodiments 2 or 42-110, wherein [N4]-[N5] corresponds to positions 600-605 of SEQ ID NO: 5, 8, or 3636.
112. The AAV capsid variant of any one of embodiments 2 or 42-111, wherein [N1]-[N2]-[N3]-[N4]-[N5] replaces positions 587-598 (e.g., A587, Q588, A589, Q590, A591, Q592, T593, G594, W595, V596, Q597, N598), numbered according to SEQ ID NO: 138.
113. The AAV capsid variant of any one of embodiments 2 or 42-112, wherein [N1]-[N2]-[N3]-[N4]-[N5] is present immediately subsequent to position 586 and replaces positions 587-598 (e.g., A587, Q588, A589, Q590, A591, Q592, T593, G594, W595, V596, Q597, N598), numbered according to SEQ ID NO: 138.
114. The AAV capsid variant of any one of embodiments 2 or 42-113, wherein [N1]-[N2]-[N3]-[N4]-[N5] corresponds to positions 587-605 of SEQ ID NO: 5, 8, or 3636.
115. The AAV capsid variant of any one of embodiments 1-114, which comprises from N-terminus to C-terminus, [N1]-[N2].
116. The AAV capsid variant of any one of embodiments 2 or 14-115, which comprises from N-terminus to C-terminus, [N1]-[N2]-[N3].
117. The AAV capsid variant of any one of embodiments 2 or 29-116, which comprises from N-terminus to C-terminus, [N1]-[N2]-[N3]-[N4].
118. The AAV capsid variant of any one of embodiments 2 or 42-117, which comprises from N-terminus to C-terminus, [N1]-[N2]-[N3]-[N4]-[N5].
119. An AAV capsid variant comprising [A][B], wherein [A] comprises the amino acid sequence of PLNGA (SEQ ID NO: 3679), and [B] comprises X1, X2, X3, X4, wherein:
   (i) X1 is: V, I, L, A, F, D, or G;
   (ii) X2 is: H, N, Q, P, D, L, R, or Y;
   (iii) X3 is: L, H, I, R, or V; and
   (iv) X4 is Y; and/or wherein the AAV capsid variant comprises an amino acid modification, e.g., a conservative substitution, of any of the aforesaid amino acids in (i)-(iv);
   optionally wherein the AAV capsid variant further comprises:
   (a) one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138; and/or
   (b) one, two, or all of an amino acid other than V at position 596 (e.g., a D, F, A, E, L, G, or I), an amino acid other than Q at position 597 (e.g., P, K, R, L, H, or E), and/or an amino acid other than N at position 598 (e.g., H, S, T, P, K, I, D, or Y), relative to a reference sequence numbered according to the amino acid sequence of SEQ ID NO: 138.
120. The AAV capsid variant of embodiment 54, wherein:
   (i) X1 is V;
   (ii) X2 is H;
   (iii) X3 is L, H, or I; and
   (iv) X4 is Y.
121. The AAV capsid variant of embodiment 119 or 120, wherein [B] comprises:
   (i) VH, VN, VQ, IH, LH, VP, VD, AH, FH, DH, VL, GH, VR, VY, LY, HY, IY, RY, HL, HH, HI, NL, QL, PL, DL, HR, LL, RL, HV, or YL; or
   (ii) VH, LY, HY, IY, HL, HH, or HI.
122. The AAV capsid variant of any one of embodiments 119-121, wherein [B] comprises:
   (i) VHL, VHH, VHI, VNL, VQL, IHL, LHL, VPL, VDL, AHL, VHR, FHL, DHL, VLL, GHL, VRL, VHV, VYL, HLY, HHY, HIY, NLY, QLY, PLY, DLY, HRY, LLY, RLY, HVY, YLY;
   (ii) VHL, VHH, VHI, HLY, HHY, or HIY.
123. The AAV capsid variant of any one of embodiments 119-122, wherein [B] is or comprises:

(i)
(SEQ ID NO: 4680)
VHLY,
(SEQ ID NO: 4683)
VHHY,
(SEQ ID NO: 4681)
VHIY,
(SEQ ID NO: 4724)
VNLY,
(SEQ ID NO: 4729)
VQLY,
(SEQ ID NO: 4730)
IHLY,
(SEQ ID NO: 4727)
LHLY,
(SEQ ID NO: 4723)
VPLY,
(SEQ ID NO: 4731)
VDLY,
(SEQ ID NO: 4732)
AHLY,
(SEQ ID NO: 4725)
VHRY,
(SEQ ID NO: 4726)
FHLY,
(SEQ ID NO: 4728)
DHLY,
(SEQ ID NO: 4733)
VLLY,
(SEQ ID NO: 4734)
GHLY,
(SEQ ID NO: 4735)
VRLY,
(SEQ ID NO: 4682)
VHVY,
or
(SEQ ID NO: 4736)
VYLY;
or
(ii)
(SEQ ID NO: 4680)
VHLY,
(SEQ ID NO: 4683)
VHHY,
or
(SEQ ID NO: 4681)
VHIY.

124. The AAV capsid variant of any one of embodiments 119-123, wherein [B] is or comprises VHLY (SEQ ID NO: 4680).
125. The AAV capsid variant of any one of embodiments 119-124, wherein [A][B] comprises:

(i)
(SEQ ID NO: 3681)
PLNGAVH,
(SEQ ID NO: 5110)
PLNGAVN,
(SEQ ID NO: 5111)
PLNGAVQ,
(SEQ ID NO: 5112)
PLNGAIH,
(SEQ ID NO: 5113)
PLNGALH,
(SEQ ID NO: 5114)
PLNGAVP,
(SEQ ID NO: 5115)
PLNGAVD,
(SEQ ID NO: 5116)
PLNGAAH,
(SEQ ID NO: 5117)
PLNGAFH,
(SEQ ID NO: 5118)
PLNGADH,
(SEQ ID NO: 5119)
PLNGAVL,
(SEQ ID NO: 5120)
PLNGAGH,
(SEQ ID NO: 5121)
PLNGAVR,
or
(SEQ ID NO: 5122);
PLNGAVY,
or
(ii)
(SEQ ID NO: 3681);
PLNGAVH,

(iii) an amino acid sequence comprising any portion of an amino acid sequence in (i) or (ii), e.g., any 2, 3, 4, 5, or 6 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
126. The AAV capsid variant of embodiment 119-125, wherein [A][B] is or comprises:

| | (i) |
|---|---|
| (SEQ ID NO: 3648) | |
| | PLNGAVHLY, |
| (SEQ ID NO: 4796) | |
| | PLNGAVHHY, |
| (SEQ ID NO: 4794) | |
| | PLNGAVHIY, |
| (SEQ ID NO: 5123) | |
| | PLNGAVNLY, |
| (SEQ ID NO: 5124) | |
| | PLNGAVQLY, |
| (SEQ ID NO: 5125) | |
| | PLNGAIHLY, |
| (SEQ ID NO: 5126) | |
| | PLNGALHLY, |
| (SEQ ID NO: 5127) | |
| | PLNGAVPLY, |
| (SEQ ID NO: 5128) | |
| | PLNGAVDLY, |
| (SEQ ID NO: 5129) | |
| | PLNGAAHLY, |
| (SEQ ID NO: 5130) | |
| | PLNGAVHRY, |
| (SEQ ID NO: 5131) | |
| | PLNGAFHLY, |
| (SEQ ID NO: 5132) | |
| | PLNGADHLY, |
| (SEQ ID NO: 5133) | |
| | PLNGAVLLY, |
| (SEQ ID NO: 5134) | |
| | PLNGAGHLY, |
| (SEQ ID NO: 5135) | |
| | PLNGAVRLY, |
| (SEQ ID NO: 5136) | |
| | PLNGAVHVY, |
| | or |
| (SEQ ID NO: 5137) | |
| | PLNGAVYLY; |
| | (ii) |
| (SEQ ID NO: 3648) | |
| | PLNGAVHLY, |
| (SEQ ID NO: 4796) | |
| | PLNGAVHHY, |
| | or |
| (SEQ ID NO: 4794) | |
| | PLNGAVHIY; |

(iii) an amino acid sequence comprising any portion of an amino acid sequence in (i) or (ii), e.g., any 2, 3, 4, 5, 6, 7, or 8 amino acids, e.g., consecutive amino acids, thereof; (iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
127. The AAV capsid variant of embodiment 119-126, wherein [A][B] is or comprises PLNGAVHLY (SEQ ID NO: 3648).
128. The AAV capsid variant of any one of embodiments 119-127, which further comprises one, two, three, or all of an amino acid other than A at position 589 (e.g., D, S, or T), an amino acid other than Q at position 590 (e.g., K, H, L, P, or R), an amino acid other than A at position 591 (e.g., P or E), and/or an amino acid other than Q at position 592 (e.g., H, K, or P), relative to a reference sequence numbered according to the amino acid sequence of SEQ ID NO: 138.
129. The AAV capsid variant of any one of embodiments 119-128, which further comprises one, two, three, or all of an amino acid other than A at position 596 (e.g., D, S, or T), an amino acid other than Q at position 597 (e.g., K, H, L, P, or R), an amino acid other than A at position 598 (e.g., P or E), and/or an amino acid other than Q at position 599 (e.g., H, K, or P), relative to a reference sequence numbered according to the amino acid sequence of SEQ ID NO: 5, 8, or 3636.
130. The AAV capsid variant of any one of embodiments 119-127, which further comprises:
   (i) A at position 589, Q at position 590, A at position 591, and/or Q at position 592, numbered according to the amino acid sequence of SEQ ID NO: 138; or
   (ii) A at position 596, Q at position 597, A at position 598, and/or Q at position 599, numbered according to the amino acid sequence of SEQ ID NO: 5, 8, or 3636.
131. The AAV capsid variant of embodiment 119-130, which further comprises [C], wherein [C] comprises X4, X5, X6, and X7, wherein:
   (a) position X4 is: A, D, S, or T;
   (b) position X5 is: Q, K, H, L, P, or R;
   (c) position X6 is: A, P, or E; and
   (d) position X7 is: Q, H, K, or P; and/or an amino acid modification, e.g., a conservative substitution, of any of the aforesaid amino acids in (a)-(d).
132. The AAV capsid variant of embodiment 131, wherein:
   (a) position X4 is: A, D, or S;
   (b) position X5 is Q or K;
   (c) position X6 is A or P; and/or
   (d) position X7 is Q.
133. The AAV capsid variant of embodiment 131 or 132, wherein [C] comprises:
   (i) AQ, AK, DQ, SQ, AH, AL, AP, AR, TQ, PQ, EQ, QA, QP, KA, HA, QE, LA, PA, or RA; or
   (ii) AQ, AK, DQ, SQ, PQ, QA, QP, or KA.
134. The AAV capsid variant of any one of embodiments 131-133, wherein [C] comprises:
   (i) AQA, AQP, AKA, DQA, SQA, AHA, AQE, ALA, APA, ARA, TQA, QAQ, QPQ, KAQ, HAQ, QEQ, QAK, LAQ, PAQ, RAQ, QAH, or QAP; or
   (ii) AQA, AQP, AKA, DQA, SQA, QAQ, QPQ, or KAQ.
135. The AAV capsid variant of any one of embodiments 131-134, wherein [C] is or comprises:

(i)
(SEQ ID NO: 4737)
AQAQ,
(SEQ ID NO: 4739)
AQPQ,
(SEQ ID NO: 4741)
AKAQ,
(SEQ ID NO: 4744)
DQAQ,
(SEQ ID NO: 4738)
SQAQ,
(SEQ ID NO: 4742)
AHAQ,
(SEQ ID NO: 4748)
AQEQ,
(SEQ ID NO: 4746)
AQAK,
(SEQ ID NO: 4749)
ALAQ,
(SEQ ID NO: 4745)
APAQ,
(SEQ ID NO: 4750)
ARAQ,
(SEQ ID NO: 4747)
AQAH,
(SEQ ID NO: 4743)
AQAP,
or
(SEQ ID NO: 4751)
TQAQ;
or
(ii)
(SEQ ID NO: 4737)
AQAQ,
(SEQ ID NO: 4739)
AQPQ,
(SEQ ID NO: 4741)
AKAQ,
(SEQ ID NO: 4744)
DQAQ,
or
(SEQ ID NO: 4738)
SQAQ.

136. The AAV capsid variant of any one of embodiments 131-135, wherein [C] is or comprises AQAQ (SEQ ID NO: 4737).
137. The AAV capsid variant of any one of embodiments 131-136, wherein [B][C] is or comprises:

(i)
(SEQ ID NO: 4797)
VHLYAQAQ,
(SEQ ID NO: 4804)
VHHYAQAQ,
(SEQ ID NO: 4798)
VHLYAQPQ,
(SEQ ID NO: 4800)
VHLYAKAQ,
(SEQ ID NO: 4801)
VHLYDQAQ,
(SEQ ID NO: 4799)
VHLYSQAQ,
(SEQ ID NO: 4802)
VHIYAQAQ,
(SEQ ID NO: 5138)
VHLYAHAQ,
(SEQ ID NO: 5139)
VNLYAQAQ,
(SEQ ID NO: 5140)
VQLYAQAQ,
(SEQ ID NO: 5141)
VHLYAQEQ,
(SEQ ID NO: 5142)
IHLYAQAQ,
(SEQ ID NO: 5143)
LHLYAQAQ,
(SEQ ID NO: 5144)
VPLYAQAQ,
(SEQ ID NO: 5145)
VHLYAQAK,
(SEQ ID NO: 5146)
VDLYAQAQ,
(SEQ ID NO: 5147)
AHLYAQAQ,
(SEQ ID NO: 5148)
VHRYAQAQ,
(SEQ ID NO: 5149)
FHLYAQAQ,
(SEQ ID NO: 5150)
VHLYALAQ,
(SEQ ID NO: 5151)
DHLYAQAQ,
(SEQ ID NO: 5152)
VHLYAPAQ,
(SEQ ID NO: 5153)
VHLYARAQ,
(SEQ ID NO: 5154)
VHLYAQAH,
(SEQ ID NO: 5155)
VLLYAQAQ,
(SEQ ID NO: 5156)
VHLYAQAP,
(SEQ ID NO: 5157)
GHLYAQAQ,
(SEQ ID NO: 5158)
VRLYAQAQ,
(SEQ ID NO: 4803)
VHVYAQAQ,
(SEQ ID NO: 5159)
VYLYAQAQ,
or
(SEQ ID NO: 5160)
VHLYTQAQ;
(ii)
(SEQ ID NO: 4797)
VHLYAQAQ,
(SEQ ID NO: 4804)
VHHYAQAQ,
(SEQ ID NO: 4798)
VHLYAQPQ,
(SEQ ID NO: 4800)
VHLYAKAQ,
(SEQ ID NO: 4801)
VHLYDQAQ,
(SEQ ID NO: 4799)
VHLYSQAQ,
or
(SEQ ID NO: 4802)
VHIYAQAQ;

(iii) an amino acid sequence comprising any portion of an amino acid sequence in (i) or (ii), e.g., any 2, 3, 4, 5, 6, or 7 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
138. The AAV capsid variant of any one of embodiments 131-137, wherein [B][C] is or comprises VHLYAQAQ (SEQ ID NO: 4797).
139. The AAV capsid variant of any one of embodiments 131-138, wherein [A][B][C] comprises:

(i)
(SEQ ID NO: 4813)
PLNGAVHLYAQ,
(SEQ ID NO: 4826)
PLNGAVHHYAQ,
(SEQ ID NO: 4812)
PLNGAVHLYAK,
(SEQ ID NO: 4814)
PLNGAVHLYDQ,
(SEQ ID NO: 4815)
PLNGAVHLYSQ,
(SEQ ID NO: 4824)
PLNGAVHIYAQ,
(SEQ ID NO: 5161)
PLNGAVHLYAH,
(SEQ ID NO: 5162)
PLNGAVNLYAQ,
(SEQ ID NO: 5163)
PLNGAVQLYAQ,
(SEQ ID NO: 5164)
PLNGAIHLYAQ,
(SEQ ID NO: 5165)
PLNGALHLYAQ,
(SEQ ID NO: 5166)
PLNGAVPLYAQ,
(SEQ ID NO: 5167)
PLNGAVDLYAQ,
(SEQ ID NO: 5168)
PLNGAAHLYAQ,
(SEQ ID NO: 5169)
PLNGAVHRYAQ,
(SEQ ID NO: 5170)
PLNGAFHLYAQ,
(SEQ ID NO: 5171)
PLNGAVHLYAL,
(SEQ ID NO: 5172)
PLNGADHLYAQ,
(SEQ ID NO: 5173)
PLNGAVHLYAP,
(SEQ ID NO: 5174)
PLNGAVHLYAR,
(SEQ ID NO: 5175)
PLNGAVLLYAQ,
(SEQ ID NO: 5176)
PLNGAGHLYAQ,
(SEQ ID NO: 5177)
PLNGAVRLYAQ,
(SEQ ID NO: 5178)
PLNGAVHVYAQ,
(SEQ ID NO: 5179)
PLNGAVYLYAQ,
or
(SEQ ID NO: 5180),
PLNGAVHLYTQ;
(ii)
(SEQ ID NO: 4813)
PLNGAVHLYAQ,
(SEQ ID NO: 4826)
PLNGAVHHYAQ,
(SEQ ID NO: 4812)
PLNGAVHLYAK,
(SEQ ID NO: 4814)
PLNGAVHLYDQ,
(SEQ ID NO: 4815)
PLNGAVHLYSQ,
or
(SEQ ID NO: 4824)
PLNGAVHIYAQ;

(iii) an amino acid sequence comprising any portion of an amino acid sequence in (i) or (ii), e.g., any 2, 3, 4, 5, 6, 7, 8, 9, or 10 amino acids, e.g., consecutive amino acids, thereof; (iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
140. The AAV capsid variant of any one of embodiments 131-139, wherein [A][B][C] is or comprises:

(i)
(SEQ ID NO: 4836)
PLNGAVHLYAQAQ,
(SEQ ID NO: 4850)
PLNGAVHHYAQAQ,
(SEQ ID NO: 4837)
PLNGAVHLYAQPQ,
(SEQ ID NO: 4835)
PLNGAVHLYAKAQ,
(SEQ ID NO: 4838)
PLNGAVHLYDQAQ,
(SEQ ID NO: 4839)
PLNGAVHLYSQAQ,
(SEQ ID NO: 4848)
PLNGAVHIYAQAQ,
(SEQ ID NO: 5181)
PLNGAVHLYAHAQ,
(SEQ ID NO: 5182)
PLNGAVNLYAQAQ,
(SEQ ID NO: 5183)
PLNGAVQLYAQAQ,
(SEQ ID NO: 5184)
PLNGAVHLYAQEQ,
(SEQ ID NO: 5185)
PLNGAIHLYAQAQ,
(SEQ ID NO: 5186)
PLNGALHLYAQAQ,
(SEQ ID NO: 5187)
PLNGAVPLYAQAQ,
(SEQ ID NO: 5188)
PLNGAVHLYAQAK,
(SEQ ID NO: 5189)
PLNGAVDLYAQAQ,
(SEQ ID NO: 5190)
PLNGAAHLYAQAQ,
(SEQ ID NO: 5191)
PLNGAVHRYAQAQ,
(SEQ ID NO: 5192)
PLNGAFHLYAQAQ,
(SEQ ID NO: 5193)
PLNGAVHLYALAQ,
(SEQ ID NO: 5194)
PLNGADHLYAQAQ,
(SEQ ID NO: 5195)
PLNGAVHLYAPAQ,
(SEQ ID NO: 5196)
PLNGAVHLYARAQ,
(SEQ ID NO: 5197)
PLNGAVHLYAQAH,
(SEQ ID NO: 5198)
PLNGAVLLYAQAQ,
(SEQ ID NO: 5199)
PLNGAVHLYAQAP,
(SEQ ID NO: 5200)
PLNGAGHLYAQAQ,
(SEQ ID NO: 5201)
PLNGAVRLYAQAQ,
(SEQ ID NO: 5202)
PLNGAVHVYAQAQ,
(SEQ ID NO: 5203)
PLNGAVYLYAQAQ,
or
(SEQ ID NO: 5204)
PLNGAVHLYTQAQ;
(ii)
(SEQ ID NO: 4836)
PLNGAVHLYAQAQ,
(SEQ ID NO: 4850)
PLNGAVHHYAQAQ,
(SEQ ID NO: 4837)
PLNGAVHLYAQPQ,
(SEQ ID NO: 4835)
PLNGAVHLYAKAQ,
(SEQ ID NO: 4838)
PLNGAVHLYDQAQ,
(SEQ ID NO: 4839)
PLNGAVHLYSQAQ,
or
(SEQ ID NO: 4848)
PLNGAVHIYAQAQ;

(iii) an amino acid sequence comprising any portion of an amino acid sequence in (i) or (ii), e.g., any 2, 3, 4, 5, 6, 7, 8, 9, 10, 11, or 12 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
141. The AAV capsid variant of any one of embodiments 131-140, wherein [A][B][C] is or comprises PLNGAVHLYAQAQ (SEQ ID NO: 4836).
142. The AAV capsid variants of any one of 119-141, which further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
143. The AAV capsid variants of any one of 119-142, which further comprises one, two, or all of an amino acid other than T at position 600 (e.g., a V, S, L, R, I, A, N, C, Q, M, P, or K), an amino acid other than G at position 601 (e.g., T, M, A, K, S, Q, V, I, R, N, P, L, H, or Y), and/or an amino acid other than W at position 602 (e.g., K, Q, S, P, C, A, G, N, T, R, V, M, H, L, E, F, or Y), relative to a reference sequence numbered according to the amino acid sequence of SEQ ID NO: 5, 8, 3636.
144. The AAV capsid variants of any one of 119-143, which further comprises one, two, three or all of:
   (i) the amino acid V, S, L, R, I, A, N, C, Q, M, P, or K (e.g., L) at position 593 numbered according to SEQ ID NO: 138 or at position 600 numbered according to SEQ ID NO: 5, 8, or 3636);
   (ii) the amino acid T, M, A, K, S, Q, V, I, R, N, P, L, H, or Y (e.g., S) at position 594 numbered according to SEQ ID NO: 138, or at position 601 numbered according to SEQ ID NO: 5, 8, or 3636; and/or
   (iii) the amino acid K, Q, S, P, C, A, G, N, T, R, V, M, H, L, E, F, or Y (e.g. P) at position 595 numbered according to SEQ ID NO: 138 or at position 602 numbered according to SEQ ID NO: 5, 8, or 3636).
145. The AAV capsid variants of any one of 119-144, which further comprises:
   (i) the amino acid L at position 593 numbered according to SEQ ID NO: 138 or at position 600 numbered according to SEQ ID NO: 5, 8, or 3636);
   (ii) the amino acid S at position 594 numbered according to SEQ ID NO: 138, or at position 601 numbered according to SEQ ID NO: 5, 8, or 3636; and
   (iii) the amino acid P at position 595 numbered according to SEQ ID NO: 138 or at position 602 numbered according to SEQ ID NO: 5, 8, or 3636).
146. The AAV capsid variants of any one of 119-141, which further comprises:
   (i) T at position 593, G at position 594, and/or W at position 595, numbered according to SEQ ID NO: 138;
   (ii) T at position 600, G at position 601, and/or W at position 602, numbered according to SEQ ID NO: 5, 8, or 3636.
147. The AAV capsid variant of any one of embodiments 119-146, which further comprises [D], wherein [D] comprises X8, X9, and X10, wherein:
   (a) position X8 is: T, V, S, L, R, I, A, N, C, Q, M, P, or K;
   (b) position X9 is: T, M, A, G, K, S, Q, V, I, R, N, P, L, H, or Y; and
   (c) position X10 is: K, Q, W, S, P, C, A, G, N, T, R, V, M, H, L, E, F, or Y; and/or an amino acid modification, e.g., a conservative substitution, of any of the aforesaid amino acids in (a)-(c).
148. The AAV capsid variant of embodiment 147, wherein:
   (a) position X8 is: T, V, S, L, R, I, A, N, C, Q, or M;
   (b) position X9 is: T, M, A, G, K, S, Q, V, I, R, N, P, L, or H; and/or
   (c) position X10 is: K, Q, W, S, P, C, A, G, N, T, R, V, M, H, L, or E.
149. The AAV capsid variant of embodiment 147 or 148, wherein:
   (a) position X8 is: T or L;
   (b) position X9 is: G or S; and/or
   (c) position X10 is: W or P.
150. The AAV capsid variant of any one of embodiments 147-149, wherein [D] comprises:
   (i) TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, PG, QS, RY, SQ, NQ, LL, LP, AQ, TY, NL, SP, LV, KG, VP, AV, KS, AM, SL, AL, RP, IP, MK, AW, GS, KQ, AP, SK, AK, GC, QK, MQ, QP, GP, QQ, AN, GK, QR, PP, AR, GG, MS, NP, KP, MN, KA, SN, MP, HP, GN, RW, MT, SR, GW, QH, GL, QM, VW, MG, AH, QT, GR, SH, GQ, GT, GA, NG, QN, VE, MM, QL, QG, YS, GM, LR, AF, PQ, SW, QW, YA, ML, GF, PA, PS, PT, GY, GV, PW, PR; or
   (ii) TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, MK, AQ, AW, GS, KQ, AP, SK, AK, GC, QK, SP, MQ, SQ, QP, RP, GP, NQ, QQ, AN, GK, QS, QR, PP, AR, GG, MS, NP, KP, MN, KS, KA, SN, MP, HP, GN, RW, MT, AM, SR, GW, QH, GL, AV, QM, VW, MG, AL, AH, SL, QT, GR, SH, LP, GQ, GT, GA, NG, QN, IP, or VE.
151. The AAV capsid variant of any one of embodiments 147-150, wherein [D] is or comprises:
   (i) TGW, LSP, TTK, TMK, VAQ, TAW, TGS, VKQ, SAP, LSK, LAP, LAQ, TAK, SAK, TGC, TQK, TVA, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG; or
   (ii) TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, or LMS.
152. The AAV capsid variant of any one of embodiments 147-151, wherein [D] is or comprises TGW.
153. The AAV capsid variant of any one of embodiments 147-151, wherein [D] is or comprises LSP.
154. The AAV capsid variant of any one of embodiments 119-153, which further comprises one, two, or all of an amino acid other than V at position 596 (e.g., D, F, A, E, L, G, or I), an amino acid other than Q at position 597 (e.g., R, P, K, L, H, or E), and/or an amino acid other than N at position 598 (e.g., H, S, T, P, K, I, D, or Y), relative to a reference sequence numbered according to the amino acid sequence of SEQ ID NO: 138.
155. The AAV capsid variant of any one of embodiments 119-154, which further comprises one, two, or all of an amino acid other than V at position 603 (e.g., D, F, A, E, L, G, or I), an amino acid other than Q at position 604 (e.g., R, P, K, L, H, or E), and/or an amino acid other than N at position 605 (e.g., H, S, T, P, K, I, D, or Y), relative to a reference sequence numbered according to the amino acid sequence of SEQ ID NO: 5, 8, or 3636.
156. The AAV capsid variant of any one of embodiments 119-155, which further comprises one, two, or all of:
   (i) the amino acid D, F, A, E, L, G, or I at position 596, numbered according to SEQ ID NO: 138, or at position 603 numbered according to SEQ ID NO: 5, 8, or 3636;
   (ii) the amino acid R, P, K, L, H, or E at position 597, numbered according to SEQ ID NO: 138, or at position 604 numbered according to SEQ ID NO: 5, 8, or 3636; and/or
   (iii) the amino acid H, S, T, P, K, I, D, or Y at position 598, numbered according to SEQ ID NO: 138, or at position 605 numbered according to SEQ ID NO: 5, 8, or 3636.
157. The AAV capsid variant of any one of embodiments 119-156, which further comprises P, K, E, or H at position 597 numbered according to SEQ ID NO: 138 or at position 604, numbered according to SEQ ID NO: 5, 8, or 3636.
158. The AAV capsid variant of any one of embodiments 119-157, which further comprises one, two, or all of:
   (i) the amino acid V at position 596 numbered according to SEQ ID NO: 138 or at position 603 numbered according to SEQ ID NO: 5, 8, or 3636;
   (ii) the amino acid K, P, E or H at position 597 numbered according to SEQ ID NO: 138 or at position 604 numbered according to SEQ ID NO: 5, 8, or 3636; and
   (iii) the amino acid N at position 598 numbered according to the amino acid sequence of SEQ ID NO: 138 or at position 605 numbered according to SEQ ID NO: 5, 8, or 3636.
159. The AAV capsid variant of any one of embodiments 119-158, which further comprises [E], wherein [E] comprises X11, X12, and X13, wherein:
   (a) position X11 is: V, D, F, A, E, L, G, or I;
   (b) position X12 is: Q, R, P, K, L, H, or E; and
   (c) position X13 is: N, H, S, T, P, K, I, D, or Y; and/or an amino acid modification, e.g., a conservative substitution, of any of the aforesaid amino acids in (a)-(c).
160. The AAV capsid variant of embodiment 159, wherein:
   (a) position X11 is: V, D, F, A, E, L, or G;
   (b) position X12 is: Q, R, P, K, or L; and/or
   (c) position X13 is: N, H, S, T, P, K, I, or D.
161. The AAV capsid variant of embodiment 159 or 160, wherein:
   (a) position X11 is V;
   (b) position X12: Q, R, P, K, or L; and/or
   (c) position X13 is: N.
162. The AAV capsid variant of any one of embodiments 159-161, wherein position X12 is P.
163. The AAV capsid variant of any one of embodiments 159-161, wherein position X12 is K.
164. The AAV capsid variant of any one of embodiments 159-161, wherein position X12 is E or H.
165. The AAV capsid variant of any one of embodiments 159-164, wherein [E] comprises:
   (i) VQ, DQ, FQ, VR, VP, VK, AQ, EQ, LQ, GQ, VL, VH, VE, DK, GH, IQ, QN, QH, QS, QT, QP, RN, PN, KN, QK, QI, LN, QD, HN, KT, KK, EN, QY, or PH; or
   (ii) VQ, DQ, FQ, VR, VP, VK, AQ, EQ, LQ, GQ, VL, QN, QH, QS, QT, QP, RN, PN, KN, QK, QI, LN, or QD.
166. The AAV capsid variant of any one of embodiments 159-165, wherein [E] is or comprises:
   (i) VQN, VPN, VKN, DQN, VQH, FQN, VQS, VQT, VQP, VRN, AQN, VQK, EQN, VQI, LQN, GQT, VLN, VQD, VHN, GQN, VKT, VKK, FQK, VEN, VQY, DKN, GHN, IQN, or VPH; or
   (ii) VQN, VPN, VKN, DQN, VQH, FQN, VQS, VQT, VQP, VRN, AQN, VQK, EQN, VQI, LQN, GQT, VLN, or VQD.
167. The AAV capsid variant of any one of embodiments 159-166, wherein [E] is or comprises VQN, VPN, or VKN.
168. The AAV capsid variant of any one of embodiments 159-167, wherein [E] is or comprises VEN, VHN, VKN, or VPN.
169. The AAV capsid variant of any one of embodiments 159-168, wherein [D][E] is or comprises:

(i)
(SEQ ID NO: 4851)
TGWVQN,
(SEQ ID NO: 5000)
TGWVPN,
(SEQ ID NO: 5047)
TTKVQN,
(SEQ ID NO: 5013)
TMKVQN,
(SEQ ID NO: 5062)
VAQVQN,
(SEQ ID NO: 4978)
TAWDQN,
(SEQ ID NO: 4992)
TGSVQH,
(SEQ ID NO: 5072)
VKQVQN,
(SEQ ID NO: 4854)
SAPVQN,
(SEQ ID NO: 4912)
LSKVQN,
(SEQ ID NO: 4891)
LAPVQN,
(SEQ ID NO: 4893)
LAQVQN,
(SEQ ID NO: 4968)
TAKVQN,
(SEQ ID NO: 4955)
SAKVQN,
(SEQ ID NO: 4982)
TGCFQN,
(SEQ ID NO: 5026)
TQKVQN,
(SEQ ID NO: 5052)
TVAVQN,
(SEQ ID NO: 4914)
LSPVQN,
(SEQ ID NO: 5050)
TTQVQN,
(SEQ ID NO: 4973)
TAQVQN,
(SEQ ID NO: 4934)
RIAVQN,
(SEQ ID NO: 4931)
RASVQN,
(SEQ ID NO: 5048)
TTPVQN,
(SEQ ID NO: 4853)
LTPVQN,
(SEQ ID NO: 4963)
STPVQN,
(SEQ ID NO: 4861)
TSPVQN,
(SEQ ID NO: 5016)
TMQVQN,
(SEQ ID NO: 5036)
TSKVQN,
(SEQ ID NO: 5092)
VSQVQN,
(SEQ ID NO: 4868)
VSPVQN,
(SEQ ID NO: 5055)
TVQVQN,
(SEQ ID NO: 5097)
VTAVQN,
(SEQ ID NO: 4941)
RQPVQN,
(SEQ ID NO: 4884)
ISGVQN,
(SEQ ID NO: 5087)
VRPVQN,
(SEQ ID NO: 4897)
LGPVQN,
(SEQ ID NO: 5022)
TNQVQN,
(SEQ ID NO: 5084)
VQQVQN,
(SEQ ID NO: 5061)
VANVQN,
(SEQ ID NO: 4870)
AAPVQN,
(SEQ ID NO: 4947)
RSTVQN,
(SEQ ID NO: 5011)
TMAVQN,
(SEQ ID NO: 4882)
IQPVQN,
(SEQ ID NO: 4878)
IASVQN,
(SEQ ID NO: 4867)
TVSVQN,
(SEQ ID NO: 4933)
RGSVQN,
(SEQ ID NO: 4925)
NSPVQN,
(SEQ ID NO: 4908)
LQPVQN,
(SEQ ID NO: 5098)
VTGVQN,
(SEQ ID NO: 5076)
VMQVQN,
(SEQ ID NO: 4959)
SMAVQN,
(SEQ ID NO: 5066)
VGKVQN,
(SEQ ID NO: 4883)
IQSVQN,
(SEQ ID NO: 4874)
CSPVQN,
(SEQ ID NO: 4910)
LQRVQN,
(SEQ ID NO: 4979)
TAWVQH,
(SEQ ID NO: 5024)
TPPVQN,
(SEQ ID NO: 5099)
VTKVQN,
(SEQ ID NO: 4855)
SSPVQN,
(SEQ ID NO: 4871)
AGPVQN,
(SEQ ID NO: 4894)
LARVQN,
(SEQ ID NO: 5051)
TTTVQN,
(SEQ ID NO: 4984)
TGGFQN,
(SEQ ID NO: 5010)
TLQVQN,
(SEQ ID NO: 5018)
TMSVQN,
(SEQ ID NO: 5060)
VAKVQN,
(SEQ ID NO: 4929)
RAAVQN,
(SEQ ID NO: 5054)
TVGVQN,
(SEQ ID NO: 4907)
LNPVQN,
(SEQ ID NO: 4915)
LSQVQN,
(SEQ ID NO: 5006)
TKPVQN,
(SEQ ID NO: 5021)
TNAVQN,
(SEQ ID NO: 4896)
LATVQN,
(SEQ ID NO: 5100)
VTPVQN,
(SEQ ID NO: 5081)
VQAVQN,
(SEQ ID NO: 4860)
TTSVQN,
(SEQ ID NO: 4875)
CTPVQN,
(SEQ ID NO: 4967)
TAGVQN,
(SEQ ID NO: 5040)
TSQVQN,
(SEQ ID NO: 5014)
TMNVQN,
(SEQ ID NO: 5043)
TSTVQN,
(SEQ ID NO: 5071)
VKPVQN,
(SEQ ID NO: 4872)
ASPVQN,
(SEQ ID NO: 5058)
VAAVQN,
(SEQ ID NO: 4901)
LKSVQN,
(SEQ ID NO: 4876)
IAAVQN,
(SEQ ID NO: 4966)
TAAVQN,
(SEQ ID NO: 5005)
TKAVQN,
(SEQ ID NO: 4994)
TGSVQS,
(SEQ ID NO: 5090)
VSNVQN,
(SEQ ID NO: 4971)
TAPVQN,
(SEQ ID NO: 4904)
LMPVQN,
(SEQ ID NO: 4900)
LHPVQN,
(SEQ ID NO: 4930)
RAQVQN,
(SEQ ID NO: 4919)
LTNVQN,
(SEQ ID NO: 4952)
RTTVQN,
(SEQ ID NO: 5044)
TSVVQN,
(SEQ ID NO: 4939)
RMSVQN,
(SEQ ID NO: 5067)
VGNVQN,
(SEQ ID NO: 4905)
LMQVQN,
(SEQ ID NO: 4976)
TATVQN,
(SEQ ID NO: 5069)
VHPVQN,
(SEQ ID NO: 5091)
VSPVQT,
(SEQ ID NO: 4886)
ISSVQN,
(SEQ ID NO: 5063)
VASVQN,
(SEQ ID NO: 5032)
TRWDQN,
(SEQ ID NO: 5020)
TMTVQN,
(SEQ ID NO: 4946)
RSSVQN,
(SEQ ID NO: 4863)
TAWVQN,
(SEQ ID NO: 4949)
RTGVQN,
(SEQ ID NO: 5064)
VATVQN,
(SEQ ID NO: 5101)
VTSVQN,
(SEQ ID NO: 4869)
VSSVQN,
(SEQ ID NO: 5023)
TNSVQN,
(SEQ ID NO: 5070)
VKAVQN,
(SEQ ID NO: 4958)
SGPVQN,
(SEQ ID NO: 4989)
TGPVQN,
(SEQ ID NO: 4969)
TAMVQN,
(SEQ ID NO: 5029)
TQPVQN,
(SEQ ID NO: 5030)
TQQVQN,
(SEQ ID NO: 5093)
VSRVQN,
(SEQ ID NO: 5002)
TGWVQP,
(SEQ ID NO: 5088)
VSAVQN,
(SEQ ID NO: 5074)
VLSVQN,
(SEQ ID NO: 5025)
TQHVQN,
(SEQ ID NO: 4895)
LASVQN,
(SEQ ID NO: 4928)
QAPVQN,
(SEQ ID NO: 4922)
NAQVQN,
(SEQ ID NO: 4873)
ATPVQN,
(SEQ ID NO: 5083)
VQPVQN,
(SEQ ID NO: 5045)
TTAVQN,
(SEQ ID NO: 5004)
TGWVRN,
(SEQ ID NO: 4852)
LAAVQN,
(SEQ ID NO: 5039)
TSPDQN,
(SEQ ID NO: 4943)
RSGVQN,
(SEQ ID NO: 4988)
TGGVQT,
(SEQ ID NO: 5049)
TTPVQT,
(SEQ ID NO: 4902)
LMAVQN,
(SEQ ID NO: 5015)
TMPVQN,
(SEQ ID NO: 4890)
LANVQN,
(SEQ ID NO: 5096)
VSTVQN,
(SEQ ID NO: 4957)
SAQVQN,
(SEQ ID NO: 4926)
NTPVQN,
(SEQ ID NO: 5095)
VSSVQT,
(SEQ ID NO: 5056)
TVSVKN,
(SEQ ID NO: 5102)
TGLVQN,
(SEQ ID NO: 4865)
TGSVQN,
(SEQ ID NO: 4983)
TGGAQN,
(SEQ ID NO: 4977)
TAVVQN,
(SEQ ID NO: 4937)
RLGVQN,
(SEQ ID NO: 4950)
RTLVQN,
(SEQ ID NO: 5027)
TQMVQN,
(SEQ ID NO: 4887)
ITPVQN,
(SEQ ID NO: 5057)
TVWVQK,
(SEQ ID NO: 4942)
RSAVQN,
(SEQ ID NO: 4974)
TASVQN,
(SEQ ID NO: 5012)
TMGVQN,
(SEQ ID NO: 4986)
TGGVQH,
(SEQ ID NO: 5085)
VQSVQN,
(SEQ ID NO: 4864)
TGGVQN,
(SEQ ID NO: 4885)
ISPVQN,
(SEQ ID NO: 5103)
TGWVKN,
(SEQ ID NO: 4990)
TGSAQN,
(SEQ ID NO: 4857)
TGWAQN,
(SEQ ID NO: 5019)
TMSVQT,
(SEQ ID NO: 5065)
VGGVQN,
(SEQ ID NO: 4892)
LAPVQT,
(SEQ ID NO: 4862)
TALVQN,
(SEQ ID NO: 4888)
LAGVQN,
(SEQ ID NO: 4948)
RTAVQN,
(SEQ ID NO: 4944)
RSPVQN,
(SEQ ID NO: 4859)
TLAVQN,
(SEQ ID NO: 4889)
LAHVQN,
(SEQ ID NO: 5037)
TSLVQN,
(SEQ ID NO: 4938)
RLSVQN,
(SEQ ID NO: 4903)
LMGVQN,
(SEQ ID NO: 4961)
SMQVQN,
(SEQ ID NO: 5031)
TQTVQN,
(SEQ ID NO: 4997)
TGWEQN,
(SEQ ID NO: 5068)
VGSVQN,
(SEQ ID NO: 5089)
VSGVQN,
(SEQ ID NO: 5075)
VMAVQN,
(SEQ ID NO: 4879)
IGGVQN,
(SEQ ID NO: 4877)
IAGVQN,
(SEQ ID NO: 4856)
TGRVQN,
(SEQ ID NO: 4911)
LSHVQN,
(SEQ ID NO: 5086)
VQTVQN,
(SEQ ID NO: 4866)
TGWDQN,
(SEQ ID NO: 4940)
RNSVQN,
(SEQ ID NO: 5009)
TLPVQN,
(SEQ ID NO: 5007)
TKQVQN,
(SEQ ID NO: 4898)
LGQVQN,
(SEQ ID NO: 4924)
NMQVQN,
(SEQ ID NO: 4927)
NVQVQN,
(SEQ ID NO: 4993)
TGSVQI,
(SEQ ID NO: 4932)
RGGVQN,
(SEQ ID NO: 5077)
VMSVQN,
(SEQ ID NO: 5046)
TTGVQN,
(SEQ ID NO: 4913)
LSPVQK,
(SEQ ID NO: 4916)
LSRVQN,
(SEQ ID NO: 5094)
VSSVQK,
(SEQ ID NO: 4972)
TAPVQT,
(SEQ ID NO: 4921)
MAPVQN,
(SEQ ID NO: 4881)
ILGVQN,
(SEQ ID NO: 4975)
TASVQT,
(SEQ ID NO: 4991)
TGSLQN,
(SEQ ID NO: 4996)
TGTVQN,
(SEQ ID NO: 4995)
TGSVQT,
(SEQ ID NO: 5042)
TSSVQT,
(SEQ ID NO: 5035)
TSHVQN,
(SEQ ID NO: 4935)
RIGVQN,
(SEQ ID NO: 4999)
TGWGQT,
(SEQ ID NO: 4956)
SAMVQN,
(SEQ ID NO: 5038)
TSMVQN,
(SEQ ID NO: 4960)
SMGVQN,
(SEQ ID NO: 4962)
SMSVQN,
(SEQ ID NO: 5041)
TSSVQN,
(SEQ ID NO: 5034)
TSGVQN,
(SEQ ID NO: 4981)
TGAVQN,
(SEQ ID NO: 5080)
VNSVQN,
(SEQ ID NO: 5059)
VAGVQN,
(SEQ ID NO: 4880)
IGSVQN,
(SEQ ID NO: 4899)
LGSVQN,
(SEQ ID NO: 5079)
VNGVQN,
(SEQ ID NO: 4918)
LTAVQN,
(SEQ ID NO: 5082)
VQNVQN,
(SEQ ID NO: 5008)
TKSVQN,
(SEQ ID NO: 4965)
SVGVQN,
(SEQ ID NO: 4980)
TAWVQT,
(SEQ ID NO: 4923)
NASVQN,
(SEQ ID NO: 5033)
TSAVQN,
(SEQ ID NO: 5017)
TMSVKN,
(SEQ ID NO: 4970)
TANVQN,
(SEQ ID NO: 4998)
TGWFQN,
(SEQ ID NO: 4985)
TGGVLN,
(SEQ ID NO: 4920)
LTSVQN,
(SEQ ID NO: 5003)
TGWVQT,
(SEQ ID NO: 4945)
RSQVQN,
(SEQ ID NO: 4936)
RIPVQN,
(SEQ ID NO: 5001)
TGWVQD,
(SEQ ID NO: 4953)
RVEVQN,
(SEQ ID NO: 5073)
VLPVQN,
(SEQ ID NO: 4987)
TGGVQK,
(SEQ ID NO: 4964)
SVAVQN,
(SEQ ID NO: 4909)
LQQVQN,
(SEQ ID NO: 4917)
LSTVQN,
(SEQ ID NO: 4954)
SAAVQN,
(SEQ ID NO: 4951)
RTSVQN,
(SEQ ID NO: 5028)
TQNVQN,
(SEQ ID NO: 5078)
VNAVQN,
(SEQ ID NO: 5053)
TVAVQT,
(SEQ ID NO: 4906)
LMSVQN,
(SEQ ID NO: 5105)
TGWVQH,
(SEQ ID NO: 4858)
TGWVQS,
(SEQ ID NO: 5205)
TMMVQN,
(SEQ ID NO: 5206)
TGGVQS,
(SEQ ID NO: 5207)
TGSFQN,
(SEQ ID NO: 5208)
RSVVQN,
(SEQ ID NO: 5209)
TGSVQK,
(SEQ ID NO: 5210)
TQLVQN,
(SEQ ID NO: 5211)
TGGVHN,
(SEQ ID NO: 5212)
RTPVQN,
(SEQ ID NO: 5213)
RQQVQN,
(SEQ ID NO: 5214)
TGSVRN,
(SEQ ID NO: 5215)
VQGVQN,
(SEQ ID NO: 5216)
PGWVQT,
(SEQ ID NO: 5217)
STQVQN,
(SEQ ID NO: 5218)
QSPVQN,
(SEQ ID NO: 5219)
RYSVQN,
(SEQ ID NO: 5220)
TQRVQN,
(SEQ ID NO: 5221)
SAGVQN,
(SEQ ID NO: 5222)
SAPVQT,
(SEQ ID NO: 5223)
RQSVQN,
(SEQ ID NO: 5224)
SQPVQN,
(SEQ ID NO: 5225)
VASVKN,
(SEQ ID NO: 5226)
TAWVRN,
(SEQ ID NO: 5227)
TGGGQN,
(SEQ ID NO: 5228)
STSVQN,
(SEQ ID NO: 5229)
VLGVQN,
(SEQ ID NO: 5230)
NQPVQN,
(SEQ ID NO: 5231)
LGTVQN,
(SEQ ID NO: 5232)
RAGVQN,
(SEQ ID NO: 5233)
TGGVKN,
(SEQ ID NO: 5234)
TAWLQN,
(SEQ ID NO: 5235)
TRWVQK,
(SEQ ID NO: 5236)
LAPVKN,
(SEQ ID NO: 5237)
TGSVQD,
(SEQ ID NO: 5238)
TGMVQN,
(SEQ ID NO: 5239)
LSNVQN,
(SEQ ID NO: 5240)
RLPVQN,
(SEQ ID NO: 5241)
RQGVQN,
(SEQ ID NO: 5242)
STPVQT,
(SEQ ID NO: 5243)
TTPVKN,
(SEQ ID NO: 5244)
RLTVQN,
(SEQ ID NO: 5245)
TLRVQN,
(SEQ ID NO: 5246)
SAFVQN,
(SEQ ID NO: 5247)
SVQVQN,
(SEQ ID NO: 5248)
LLPVQN,
(SEQ ID NO: 5249)
RTQVQN,
(SEQ ID NO: 5250)
TGSDQN,
(SEQ ID NO: 5251)
VASDQN,
(SEQ ID NO: 5252)
VSPVKN,
(SEQ ID NO: 5253)
LPPVQN,
(SEQ ID NO: 5254)
SSPVQT,
(SEQ ID NO: 5255)
AQPVQN,
(SEQ ID NO: 5256)
TPQVQN,
(SEQ ID NO: 5257)
TSWVQN,
(SEQ ID NO: 5258)
TGGDQN,
(SEQ ID NO: 5109)
LSPVKN,
(SEQ ID NO: 5259)
SSPVKN,
(SEQ ID NO: 5260)
NTTVQN,
(SEQ ID NO: 5261)
TTRVQN,
(SEQ ID NO: 5262)
TQWVQN,
(SEQ ID NO: 5263)
TGSVHN,
(SEQ ID NO: 5264)
TGGLQN,
(SEQ ID NO: 5265)
TAWVQK,
(SEQ ID NO: 5266)
TGRVQT,
(SEQ ID NO: 5267)
NTQVQN,
(SEQ ID NO: 5268)
TGWLQN,
(SEQ ID NO: 5269)
TYAVQN,
(SEQ ID NO: 5270)
TLSVQN,
(SEQ ID NO: 5271)
NLPVQN,
(SEQ ID NO: 5272)
TSSDQN,
(SEQ ID NO: 5273)
ATSVQN,
(SEQ ID NO: 5274)
TAWFQN,
(SEQ ID NO: 5275)
ATQVQN,
(SEQ ID NO: 5276)
VSSVKN,
(SEQ ID NO: 5107)
LSSVQN,
(SEQ ID NO: 5277)
TGSGQN,
(SEQ ID NO: 5278)
LQPVQT,
(SEQ ID NO: 5279)
VSAVKN,
(SEQ ID NO: 5280)
TQAVQN,
(SEQ ID NO: 5281)
TGWVQK,
(SEQ ID NO: 5282)
VMPVQN,
(SEQ ID NO: 5283)
TVSVQK,
(SEQ ID NO: 5284)
TAWAQN,
(SEQ ID NO: 5285)
NALVQN,
(SEQ ID NO: 5286)
RMLVQN,
(SEQ ID NO: 5287)
TVAVKN,
(SEQ ID NO: 5288)
RQLVQN,
(SEQ ID NO: 5289)
TLGVQN,
(SEQ ID NO: 5290)
LGPVQT,
(SEQ ID NO: 5291)
TGSVKN,
(SEQ ID NO: 5292)
TMSDQN,
(SEQ ID NO: 5293)
LASVKN,
(SEQ ID NO: 5294)
TGFVQN,
(SEQ ID NO: 5295)
SALVQN,
(SEQ ID NO: 5296)
TGWVKT,
(SEQ ID NO: 5297)
SQLVQN,
(SEQ ID NO: 5298)
TGWGQN,
(SEQ ID NO: 5299)
LSAVQN,
(SEQ ID NO: 5300)
TMQVQT,
(SEQ ID NO: 5301)
TGQVQN,
(SEQ ID NO: 5302)
TSPVKN,
(SEQ ID NO: 5303)
LSQVQT,
(SEQ ID NO: 5304)
TGSVLN,
(SEQ ID NO: 5305)
TNGVQN,
(SEQ ID NO: 5306)
TGWVKK,
(SEQ ID NO: 5307)
AAAVQN,
(SEQ ID NO: 5308)
SAVVQN,
(SEQ ID NO: 5309)
PGWVQH,
(SEQ ID NO: 5310)
TASDQN,
(SEQ ID NO: 5106)
LSGVQN,
(SEQ ID NO: 5311)
SSRVQN,
(SEQ ID NO: 5312)
SPPVQN,
(SEQ ID NO: 5313)
VQPVQT,
(SEQ ID NO: 5314)
TSSVKN,
(SEQ ID NO: 5315)
LSPLQN,
(SEQ ID NO: 5316)
VSQVQK,
(SEQ ID NO: 5317)
LVGVQN,
(SEQ ID NO: 5318)
TLSVKN,
(SEQ ID NO: 5319)
TGWFQK,
(SEQ ID NO: 5320)
TPAVQN,
(SEQ ID NO: 5321)
TVGVKN,
(SEQ ID NO: 5322)
KGWDQN,
(SEQ ID NO: 5323)
TAWVLN,
(SEQ ID NO: 5324)
VPPVQN,
(SEQ ID NO: 5325)
ATGVQN,
(SEQ ID NO: 5326)
TGGVQI,
(SEQ ID NO: 5327)
TGWVLN,
(SEQ ID NO: 5328)
TAWGQN,
(SEQ ID NO: 5329)
TGWVHN,
(SEQ ID NO: 5330)
LGSVQT,
(SEQ ID NO: 5331)
SANVQN,
(SEQ ID NO: 5332)
TGGVQD,
(SEQ ID NO: 5333)
TMAVKN,
(SEQ ID NO: 5334)
TASVKN,
(SEQ ID NO: 5335)
SSPVQK,
(SEQ ID NO: 5336)
TGTVQT,
(SEQ ID NO: 5337)
TGWVQI,
(SEQ ID NO: 5338)
TVWVKN,
(SEQ ID NO: 5339)
SQQVQN,
(SEQ ID NO: 5340)
VGSVQT,
(SEQ ID NO: 5341)
SSMVQN,
(SEQ ID NO: 5342)
TSPVQK,
(SEQ ID NO: 5343)
AVGVQN,
(SEQ ID NO: 5344)
VAPVQN,
(SEQ ID NO: 5345)
TLPVQK,
(SEQ ID NO: 5346)
TGRVQH,
(SEQ ID NO: 5347)
TPSVQN,
(SEQ ID NO: 5348)
TGWVEN,
(SEQ ID NO: 5349)
RGWVQN,
(SEQ ID NO: 5350)
TGSVEN,
(SEQ ID NO: 5351)
SSLVQN,
(SEQ ID NO: 5352)
TAWVKN,
(SEQ ID NO: 5353)
TYSVQN,
(SEQ ID NO: 5354)
LAAVQT,
(SEQ ID NO: 5355)
TALVKN,
(SEQ ID NO: 5356)
TGWVQY,
(SEQ ID NO: 5357)
TLPVQT,
(SEQ ID NO: 5358)
TGLVQH,
(SEQ ID NO: 5359)
TPTVQN,
(SEQ ID NO: 5360)
TASVQK,
(SEQ ID NO: 5361)
TSPVQI,
(SEQ ID NO: 5362)
IGWVQN,
(SEQ ID NO: 5363)
TGWDKN,
(SEQ ID NO: 5364)
KSSVQN,
(SEQ ID NO: 5365)
TGYVQN,
(SEQ ID NO: 5366)
RGWVQT,
(SEQ ID NO: 5367)
RSLVQN,
(SEQ ID NO: 5368)
TGGVEN,
(SEQ ID NO: 5369)
TGCVRN,
(SEQ ID NO: 5370)
LSPVQS,
(SEQ ID NO: 5371)
TGPVQT,
(SEQ ID NO: 5372)
TVGVQK,
(SEQ ID NO: 5373)
TASGQN,
(SEQ ID NO: 5374)
SVSVQN,
(SEQ ID NO: 5375)
SGPVQT,
(SEQ ID NO: 5376)
VMSVKN,
(SEQ ID NO: 5377)
LGSVQK,
(SEQ ID NO: 5378)
TGLVLN,
(SEQ ID NO: 5379)
TSNVQN,
(SEQ ID NO: 5380)
TGWGHN,
(SEQ ID NO: 5381)
SQMVQN,
(SEQ ID NO: 5382)
TVSVHN,
(SEQ ID NO: 5383)
LSSVQT,
(SEQ ID NO: 5384)
TASVRN,
(SEQ ID NO: 5385)
VPAVQN,
(SEQ ID NO: 5386)
TGRVQK,
(SEQ ID NO: 5387)
AMSVQN,
(SEQ ID NO: 5388)
TAWVHN,
(SEQ ID NO: 5389)
TGLVRN,
(SEQ ID NO: 5390)
RTLVQT,
(SEQ ID NO: 5391)
TGSIQN,
(SEQ ID NO: 5392)
LSSVKN,
(SEQ ID NO: 5393)
TLQVQK,
(SEQ ID NO: 5394)
VGSVKN,
(SEQ ID NO: 5395)
LAPLQN,
(SEQ ID NO: 5396)
TPGVQN,
(SEQ ID NO: 5397)
LSAVQT,
(SEQ ID NO: 5398)
TGVVQN,
(SEQ ID NO: 5399)
VPQVQN,
(SEQ ID NO: 5400)
TGCVQK,
(SEQ ID NO: 5401)
TRWVQT,
(SEQ ID NO: 5402)
TGLDQN,
(SEQ ID NO: 5403)
VSSVHN,
(SEQ ID NO: 5404)
KGWVQT,
(SEQ ID NO: 5405)
SLPVQN,
(SEQ ID NO: 5406)
TTSVHN,
(SEQ ID NO: 5407)
TVWVQN,
(SEQ ID NO: 5408)
TAQLQN,
(SEQ ID NO: 5409)
TRWVKN,
(SEQ ID NO: 5410)
TAWIQN,
(SEQ ID NO: 5411)
LSQVKN,
(SEQ ID NO: 5412)
TSTVKN,
(SEQ ID NO: 5413)
ALPVQN,
(SEQ ID NO: 5414)
TSMVQT,
(SEQ ID NO: 5415)
TSSVQH,
(SEQ ID NO: 5416)
TAMVKN,
(SEQ ID NO: 5417)
TPWVQN,
(SEQ ID NO: 5418)
TPRVQN,
(SEQ ID NO: 5419)
SSSVQN,
(SEQ ID NO: 5420)
RPPVQN,
(SEQ ID NO: 5421)
LAGVKN,
(SEQ ID NO: 5422)
TSPAQN,
(SEQ ID NO: 5423)
RSPVQT,
(SEQ ID NO: 5424)
TGWVPH,
(SEQ ID NO: 5425)
PGWGQN,
(SEQ ID NO: 5426)
IPPVQN,
(SEQ ID NO: 5427)
TGRVKN,
(SEQ ID NO: 5428)
TGRLQN,
(SEQ ID NO: 5429)
LSSVQH,
(SEQ ID NO: 5430)
AGWVQT,
(SEQ ID NO: 5431)
TGLVQS,
(SEQ ID NO: 5432)
TGCVQI,
(SEQ ID NO: 5433)
RPGVQN,
(SEQ ID NO: 5434)
TAAVQH,
(SEQ ID NO: 5435)
TGCDQN,
(SEQ ID NO: 5436)
TGRVRN,
or
(SEQ ID NO: 5437)
TGRDQN;
(ii)
(SEQ ID NO: 4851)
TGWVQN,
(SEQ ID NO: 5000)
TGWVPN,
(SEQ ID NO: 5047)
TTKVQN,
(SEQ ID NO: 5013)
TMKVQN,
(SEQ ID NO: 5062)
VAQVQN,
(SEQ ID NO: 4978)
TAWDQN,
(SEQ ID NO: 4992)
TGSVQH,
(SEQ ID NO: 5072)
VKQVQN,
(SEQ ID NO: 4854)
SAPVQN,
(SEQ ID NO: 4912)
LSKVQN,
(SEQ ID NO: 4891)
LAPVQN,
(SEQ ID NO: 4893)
LAQVQN,
(SEQ ID NO: 4968)
TAKVQN,
(SEQ ID NO: 4955)
SAKVQN,
(SEQ ID NO: 4982)
TGCFQN,
(SEQ ID NO: 5026)
TQKVQN,
(SEQ ID NO: 5052)
TVAVQN,
(SEQ ID NO: 4914)
LSPVQN,
(SEQ ID NO: 5050)
TTQVQN,
(SEQ ID NO: 4973)
TAQVQN,
(SEQ ID NO: 4934)
RIAVQN,
(SEQ ID NO: 4931)
RASVQN,
(SEQ ID NO: 5048)
TTPVQN,
(SEQ ID NO: 4853)
LTPVQN,
(SEQ ID NO: 4963)
STPVQN,
(SEQ ID NO: 4861)
TSPVQN,
(SEQ ID NO: 5016)
TMQVQN,
(SEQ ID NO: 5036)
TSKVQN,
(SEQ ID NO: 5092)
VSQVQN,
(SEQ ID NO: 4868)
VSPVQN,
(SEQ ID NO: 5055)
TVQVQN,
(SEQ ID NO: 5097)
VTAVQN,
(SEQ ID NO: 4941)
RQPVQN,
(SEQ ID NO: 4884)
ISGVQN,
(SEQ ID NO: 5087)
VRPVQN,
(SEQ ID NO: 4897)
LGPVQN,
(SEQ ID NO: 5022)
TNQVQN,
(SEQ ID NO: 5084)
VQQVQN,
(SEQ ID NO: 5061)
VANVQN,
(SEQ ID NO: 4870)
AAPVQN,
(SEQ ID NO: 4947)
RSTVQN,
(SEQ ID NO: 5011)
TMAVQN,
(SEQ ID NO: 4882)
IQPVQN,
(SEQ ID NO: 4878)
IASVQN,
(SEQ ID NO: 4867)
TVSVQN,
(SEQ ID NO: 4933)
RGSVQN,
(SEQ ID NO: 4925)
NSPVQN,
(SEQ ID NO: 4908)
LQPVQN,
(SEQ ID NO: 5098)
VTGVQN,
(SEQ ID NO: 5076)
VMQVQN,
(SEQ ID NO: 4959)
SMAVQN,
(SEQ ID NO: 5066)
VGKVQN,
(SEQ ID NO: 4883)
IQSVQN,
(SEQ ID NO: 4874)
CSPVQN,
(SEQ ID NO: 4910)
LQRVQN,
(SEQ ID NO: 4979)
TAWVQH,
(SEQ ID NO: 5024)
TPPVQN,
(SEQ ID NO: 5099)
VTKVQN,
(SEQ ID NO: 4855)
SSPVQN,
(SEQ ID NO: 4871)
AGPVQN,
(SEQ ID NO: 4894)
LARVQN,
(SEQ ID NO: 5051)
TTTVQN,
(SEQ ID NO: 4984)
TGGFQN,
(SEQ ID NO: 5010)
TLQVQN,
(SEQ ID NO: 5018)
TMSVQN,
(SEQ ID NO: 5060)
VAKVQN,
(SEQ ID NO: 4929)
RAAVQN,
(SEQ ID NO: 5054)
TVGVQN,
(SEQ ID NO: 4907)
LNPVQN,
(SEQ ID NO: 4915)
LSQVQN,
(SEQ ID NO: 5006)
TKPVQN,
(SEQ ID NO: 5021)
TNAVQN,
(SEQ ID NO: 4896)
LATVQN,
(SEQ ID NO: 5100)
VTPVQN,
(SEQ ID NO: 5081)
VQAVQN,
(SEQ ID NO: 4860)
TTSVQN,
(SEQ ID NO: 4875)
CTPVQN,
(SEQ ID NO: 4967)
TAGVQN,
(SEQ ID NO: 5040)
TSQVQN,
(SEQ ID NO: 5014)
TMNVQN,
(SEQ ID NO: 5043)
TSTVQN,
(SEQ ID NO: 5071)
VKPVQN,
(SEQ ID NO: 4872)
ASPVQN,
(SEQ ID NO: 5058)
VAAVQN,
(SEQ ID NO: 4901)
LKSVQN,
(SEQ ID NO: 4876)
IAAVQN,
(SEQ ID NO: 4966)
TAAVQN,
(SEQ ID NO: 5005)
TKAVQN,
(SEQ ID NO: 4994)
TGSVQS,
(SEQ ID NO: 5090)
VSNVQN,
(SEQ ID NO: 4971)
TAPVQN,
(SEQ ID NO: 4904)
LMPVQN,
(SEQ ID NO: 4900)
LHPVQN,
(SEQ ID NO: 4930)
RAQVQN,
(SEQ ID NO: 4919)
LTNVQN,
(SEQ ID NO: 4952)
RTTVQN,
(SEQ ID NO: 5044)
TSVVQN,
(SEQ ID NO: 4939)
RMSVQN,
(SEQ ID NO: 5067)
VGNVQN,
(SEQ ID NO: 4905)
LMQVQN,
(SEQ ID NO: 4976)
TATVQN,
(SEQ ID NO: 5069)
VHPVQN,
(SEQ ID NO: 5091)
VSPVQT,
(SEQ ID NO: 4886)
ISSVQN,
(SEQ ID NO: 5063)
VASVQN,
(SEQ ID NO: 5032)
TRWDQN,
(SEQ ID NO: 5020)
TMTVQN,
(SEQ ID NO: 4946)
RSSVQN,
(SEQ ID NO: 4863)
TAWVQN,
(SEQ ID NO: 4949)
RTGVQN,
(SEQ ID NO: 5064)
VATVQN,
(SEQ ID NO: 5101)
VTSVQN,
(SEQ ID NO: 4869)
VSSVQN,
(SEQ ID NO: 5023)
TNSVQN,
(SEQ ID NO: 5070)
VKAVQN,
(SEQ ID NO: 4958)
SGPVQN,
(SEQ ID NO: 4989)
TGPVQN,
(SEQ ID NO: 4969)
TAMVQN,
(SEQ ID NO: 5029)
TQPVQN,
(SEQ ID NO: 5030)
TQQVQN,
(SEQ ID NO: 5093)
VSRVQN,
(SEQ ID NO: 5002)
TGWVQP,
(SEQ ID NO: 5088)
VSAVQN,
(SEQ ID NO: 5074)
VLSVQN,
(SEQ ID NO: 5025)
TQHVQN,
(SEQ ID NO: 4895)
LASVQN,
(SEQ ID NO: 4928)
QAPVQN,
(SEQ ID NO: 4922)
NAQVQN,
(SEQ ID NO: 4873)
ATPVQN,
(SEQ ID NO: 5083)
VQPVQN,
(SEQ ID NO: 5045)
TTAVQN,
(SEQ ID NO: 5004)
TGWVRN,
(SEQ ID NO: 4852)
LAAVQN,
(SEQ ID NO: 5039)
TSPDQN,
(SEQ ID NO: 4943)
RSGVQN,
(SEQ ID NO: 4988)
TGGVQT,
(SEQ ID NO: 5049)
TTPVQT,
(SEQ ID NO: 4902)
LMAVQN,
(SEQ ID NO: 5015)
TMPVQN,
(SEQ ID NO: 4890)
LANVQN,
(SEQ ID NO: 5096)
VSTVQN,
(SEQ ID NO: 4957)
SAQVQN,
(SEQ ID NO: 4926)
NTPVQN,
(SEQ ID NO: 5095)
VSSVQT,
(SEQ ID NO: 5056)
TVSVKN,
(SEQ ID NO: 5102)
TGLVQN,
(SEQ ID NO: 4865)
TGSVQN,
(SEQ ID NO: 4983)
TGGAQN,
(SEQ ID NO: 4977)
TAVVQN,
(SEQ ID NO: 4937)
RLGVQN,
(SEQ ID NO: 4950)
RTLVQN,
(SEQ ID NO: 5027)
TQMVQN,
(SEQ ID NO: 4887)
ITPVQN,
(SEQ ID NO: 5057)
TVWVQK,
(SEQ ID NO: 4942)
RSAVQN,
(SEQ ID NO: 4974)
TASVQN,
(SEQ ID NO: 5012)
TMGVQN,
(SEQ ID NO: 4986)
TGGVQH,
(SEQ ID NO: 5085)
VQSVQN,
(SEQ ID NO: 4864)
TGGVQN,
(SEQ ID NO: 4885)
ISPVQN,
(SEQ ID NO: 5103)
TGWVKN,
(SEQ ID NO: 4990)
TGSAQN,
(SEQ ID NO: 4857)
TGWAQN,
(SEQ ID NO: 5019)
TMSVQT,
(SEQ ID NO: 5065)
VGGVQN,
(SEQ ID NO: 4892)
LAPVQT,
(SEQ ID NO: 4862)
TALVQN,
(SEQ ID NO: 4888)
LAGVQN,
(SEQ ID NO: 4948)
RTAVQN,
(SEQ ID NO: 4944)
RSPVQN,
(SEQ ID NO: 4859)
TLAVQN,
(SEQ ID NO: 4889)
LAHVQN,
(SEQ ID NO: 5037)
TSLVQN,
(SEQ ID NO: 4938)
RLSVQN,
(SEQ ID NO: 4903)
LMGVQN,
(SEQ ID NO: 4961)
SMQVQN,
(SEQ ID NO: 5031)
TQTVQN,
(SEQ ID NO: 4997)
TGWEQN,
(SEQ ID NO: 5068)
VGSVQN,
(SEQ ID NO: 5089)
VSGVQN,
(SEQ ID NO: 5075)
VMAVQN,
(SEQ ID NO: 4879)
IGGVQN,
(SEQ ID NO: 4877)
IAGVQN,
(SEQ ID NO: 4856)
TGRVQN,
(SEQ ID NO: 4911)
LSHVQN,
(SEQ ID NO: 5086)
VQTVQN,
(SEQ ID NO: 4866)
TGWDQN,
(SEQ ID NO: 4940)
RNSVQN,
(SEQ ID NO: 5009)
TLPVQN,
(SEQ ID NO: 5007)
TKQVQN,
(SEQ ID NO: 4898)
LGQVQN,
(SEQ ID NO: 4924)
NMQVQN,
(SEQ ID NO: 4927)
NVQVQN,
(SEQ ID NO: 4993)
TGSVQI,
(SEQ ID NO: 4932)
RGGVQN,
(SEQ ID NO: 5077)
VMSVQN,
(SEQ ID NO: 5046)
TTGVQN,
(SEQ ID NO: 4913)
LSPVQK,
(SEQ ID NO: 4916)
LSRVQN,
(SEQ ID NO: 5094)
VSSVQK,
(SEQ ID NO: 4972)
TAPVQT,
(SEQ ID NO: 4921)
MAPVQN,
(SEQ ID NO: 4881)
ILGVQN,
(SEQ ID NO: 4975)
TASVQT,
(SEQ ID NO: 4991)
TGSLQN,
(SEQ ID NO: 4996)
TGTVQN,
(SEQ ID NO: 4995)
TGSVQT,
(SEQ ID NO: 5042)
TSSVQT,
(SEQ ID NO: 5035)
TSHVQN,
(SEQ ID NO: 4935)
RIGVQN,
(SEQ ID NO: 4999)
TGWGQT,
(SEQ ID NO: 4956)
SAMVQN,
(SEQ ID NO: 5038)
TSMVQN,
(SEQ ID NO: 4960)
SMGVQN,
(SEQ ID NO: 4962)
SMSVQN,
(SEQ ID NO: 5041)
TSSVQN,
(SEQ ID NO: 5034)
TSGVQN,
(SEQ ID NO: 4981)
TGAVQN,
(SEQ ID NO: 5080)
VNSVQN,
(SEQ ID NO: 5059)
VAGVQN,
(SEQ ID NO: 4880)
IGSVQN,
(SEQ ID NO: 4899)
LGSVQN,
(SEQ ID NO: 5079)
VNGVQN,
(SEQ ID NO: 4918)
LTAVQN,
(SEQ ID NO: 5082)
VQNVQN,
(SEQ ID NO: 5008)
TKSVQN,
(SEQ ID NO: 4965)
SVGVQN,
(SEQ ID NO: 4980)
TAWVQT,
(SEQ ID NO: 4923)
NASVQN,
(SEQ ID NO: 5033)
TSAVQN,
(SEQ ID NO: 5017)
TMSVKN,
(SEQ ID NO: 4970)
TANVQN,
(SEQ ID NO: 4998)
TGWFQN,
(SEQ ID NO: 4985)
TGGVLN,
(SEQ ID NO: 4920)
LTSVQN,
(SEQ ID NO: 5003)
TGWVQT,
(SEQ ID NO: 4945)
RSQVQN,
(SEQ ID NO: 4936)
RIPVQN,
(SEQ ID NO: 5001)
TGWVQD,
(SEQ ID NO: 4953)
RVEVQN,
(SEQ ID NO: 5073)
VLPVQN,
(SEQ ID NO: 4987)
TGGVQK,
(SEQ ID NO: 4964)
SVAVQN,
(SEQ ID NO: 4909)
LQQVQN,
(SEQ ID NO: 4917)
LSTVQN,
(SEQ ID NO: 4954)
SAAVQN,
(SEQ ID NO: 4951)
RTSVQN,
(SEQ ID NO: 5028)
TQNVQN,
(SEQ ID NO: 5078)
VNAVQN,
(SEQ ID NO: 5053)
TVAVQT,
or
(SEQ ID NO: 4906)
LMSVQN;

(iii) an amino acid sequence comprising any portion of an amino acid sequence in (i) or (ii), e.g., any 2, 3, 4, or 5 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
170. The AAV capsid variant of any one of embodiments 159-169, wherein [D][E] is or comprises TGWVQN (SEQ ID NO: 4851), TGWVPN (SEQ ID NO: 5000), or LSPVKN (SEQ ID NO: 5109).
171. The AAV capsid variant of any one of embodiments 159-170, wherein:
   (i) [B] is or comprises: VHLY (SEQ ID NO: 4680), VHHY (SEQ ID NO: 4683), VHIY (SEQ ID NO: 4681), VNLY (SEQ ID NO: 4724), VQLY (SEQ ID NO: 4729), IHLY (SEQ ID NO: 4730), LHLY (SEQ ID NO: 4727), VPLY (SEQ ID NO: 4723), VDLY (SEQ ID NO: 4731), AHLY (SEQ ID NO: 4732), VHRY (SEQ ID NO: 4725), FHLY (SEQ ID NO: 4726), DHLY (SEQ ID NO: 4728), VLLY (SEQ ID NO: 4733), GHLY (SEQ ID NO: 4734), VRLY (SEQ ID NO: 4735), VHVY (SEQ ID NO: 4682), or VYLY (SEQ ID NO: 4736);
   (ii) [C] is or comprises: AQAQ (SEQ ID NO: 4737), AQPQ (SEQ ID NO: 4739), AKAQ (SEQ ID NO: 4741), DQAQ (SEQ ID NO: 4744), SQAQ (SEQ ID NO: 4738), AHAQ (SEQ ID NO: 4742), AQEQ (SEQ ID NO: 4748), AQAK (SEQ ID NO: 4746), ALAQ (SEQ ID NO: 4749), APAQ (SEQ ID NO: 4745), ARAQ (SEQ ID NO: 4750), AQAH (SEQ ID NO: 4747), AQAP (SEQ ID NO: 4743), or TQAQ (SEQ ID NO: 4751);
   (iii) [D] is or comprises: TGW, LSP, TTK, TMK, VAQ, TAW, TGS, VKQ, SAP, LSK, LAP, LAQ, TAK, SAK, TGC, TQK, TVA, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG; and/or
   (iv) [E] is or comprises: VQN, VPN, VKN, DQN, VQH, FQN, VQS, VQT, VQP, VRN, AQN, VQK, EQN, VQI, LQN, GQT, VLN, VQD, VHN, GQN, VKT, VKK, FQK, VEN, VQY, DKN, GHN, IQN, or VPH.
172. The AAV capsid variant of any one of embodiments 159-171, wherein:
   (i) [B] is or comprises: VHLY (SEQ ID NO: 4680), VHHY (SEQ ID NO: 4683), or VHIY (SEQ ID NO: 4681);
   (ii) [C] is or comprises: AQAQ (SEQ ID NO: 4737), AQPQ (SEQ ID NO: 4739), AKAQ (SEQ ID NO: 4741), DQAQ (SEQ ID NO: 4744), or SQAQ (SEQ ID NO: 4738);
   (iii) [D] is or comprises: TGW, LSP, TTK, TMK, VAQ, TAW, TGS, VKQ, SAP, LSK, LAP, LAQ, TAK, SAK, TGC, TQK, TVA, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, or LMS; and/or
   (iv) [E] is or comprises: VQN, VPN, VKN, DQN, VQH, FQN, VQS, VQT, VQP, VRN, AQN, VQK, EQN, VQI, LQN, GQT, VLN, or VQD.
173. The AAV capsid variant of any one of embodiments 159-172, wherein [A][B][C][D][E] comprises:
   (i) the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080,1083-1087, 1089, 1090, 1093, 1094, 1097, 1100, 1101, 1103, 1105-1107, 1110-1112, 1114-1117, 1119, 1121, 1125, 1126, 1129, 1132, 1133, 1135;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
174. The AAV capsid variant of any one of embodiments 159-173, wherein [A][B][C][D][E] comprises:
   (i) the amino acid sequence of any of SEQ ID NOs: 139, 143, 148, 149, 151, 153-158, 160-163, 166, 168, 170, 171, 173-175, 177-179, 181, 182, 184-188, 191-197, 199, 200, 201-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404, 405, 406-411, 413-415, 417, 419-429, 432, 433, 435-438, 440-442, 444-447, 450-454, 456, 458-461, 464, 465, 467-469, or 471-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
175. The AAV capsid variant of any one of embodiments 159-174, wherein [A][B][C][D][E] comprises the amino acid sequence of PLNGAVHLYAQAQTGWVPN (SEQ ID NO: 314).
176. The AAV capsid variant of any one of embodiments 159-174, wherein [A][B][C][D][E] comprises the amino acid sequence of PLNGAVHLYAQAQLSPVKN (SEQ ID NO: 566).
177. The AAV capsid variant of any one of embodiments 159-174, wherein [A][B][C][D][E] comprises the amino acid sequence the amino acid sequence of PLNGAVHLYAQAQTGWVQN (SEQ ID NO: 476).
178. The AAV capsid variant of any one of embodiments 159-177, wherein [A][B] is present in loop VIII.
179. The AAV capsid variant of any one of embodiments 159-178, wherein [C], [D], and/or [E] is present in loop VIII.
180. The AAV capsid variant of any one of embodiments 159-179, wherein [A][B][C][D][E] is present in loop VIII.
181. The AAV capsid variant of any one of embodiments 119-180, which comprises an amino acid other than A at position 587 and/or an amino acid other than Q at position 588, numbered according to SEQ ID NO: 138.
182. The AAV capsid variant of any one of embodiments 119-181, which comprises:
   (i) the amino acid P at position 587, numbered according to SEQ ID NO: 5, 8, 138, or 3636; and/or
   (ii) the amino acid L at position 588, numbered according to SEQ ID NO: 5, 8, 138, or 3636.
183. The AAV capsid variant of any one of embodiments 119-182, wherein [A] is present immediately subsequent to position 586, relative to a reference sequence numbered according to the amino acid sequence of SEQ ID NO: 5, 8, 138, or 3636.
184. The AAV capsid variant of any one of embodiments 119-183, wherein [A] is present immediately subsequent to position 586, and replaces positions 587 and 588 (e.g., A587 and Q588), numbered according to SEQ ID NO: 138.
185. The AAV capsid variant of any one of embodiments 119-184, wherein [A] replaces positions 587 and 588 (e.g., A587 and Q588), numbered according to SEQ ID NO: 138.
186. The AAV capsid variant of any one of embodiments 119-185, wherein [A] corresponds to positions 587-591 of SEQ ID NO: 5, 8, or 3636.
187. The AAV capsid variant of any one of embodiments 119-186, wherein [B] is present immediately subsequent to [A].
188. The AAV capsid variant of any one of embodiments 119-187, wherein [B] is present immediately subsequent to [A], wherein [A] is present immediately subsequent to position 586, relative to a reference sequence numbered according to the amino acid sequence of SEQ ID NO: 5, 8, 138, or 3636.
189. The AAV capsid variant of any one of embodiments 119-188, wherein [B] is present immediately subsequent to [A], wherein [A] is present immediately subsequent to position 586 and replaces positions 587 and 588 (e.g., A587 and Q588), numbered according to SEQ ID NO: 138.
190. The AAV capsid variant of any one of embodiments 119-189, wherein [B] corresponds to positions 592 to 595 of SEQ ID NO: 5, 8, or 3636.
191. The AAV capsid variant of any one of embodiments 119-190, wherein [A][B] replaces positions 587 and 588 (e.g., A587 and Q588), numbered according to SEQ ID NO: 138.
192. The AAV capsid variant of any one of embodiments 119-191, wherein [A][B] is present immediately subsequent to position 586, numbered according to SEQ ID NO: 5, 8, 138, or 3636.
193. The AAV capsid variant of any one of embodiments 119-192, wherein [A][B] is present immediately subsequent to position 586 and replaces positions 587 and 588 (e.g., A587 and Q588), numbered according to SEQ ID NO: 138.
194. The AAV capsid variant of any one of embodiments 119-193, wherein [A][B] corresponds to positions 587-595 of SEQ ID NO: 5, 8, or 3636.
195. The AAV capsid variant of any one of embodiments 131-194, wherein [C] is present immediately subsequent to position 588, relative to a reference sequence numbered according to the amino acid sequence of SEQ ID NO: 138.
196. The AAV capsid variant of any one of embodiments 131-195, wherein [C] replaces positions 589-592 (e.g., A589, Q590, A591, Q592), numbered according to the amino acid sequence of SEQ ID NO: 138.
197. The AAV capsid variant of any one of embodiments 131-196, wherein [C] is present immediately subsequent to position 588, and replaces positions 589-592 (e.g., A589, Q590, A591, Q592), numbered according to the amino acid sequence of SEQ ID NO: 138.
198. The AAV capsid variant of any one of embodiments 131-197, wherein [C] corresponds to positions 596-599 of SEQ ID NO: 5, 8, or 3636.
199. The AAV capsid variant of any one of embodiments 131-198, wherein [A][B][C] is present immediately subsequent to position 586, numbered according to SEQ ID NO: 138.
200. The AAV capsid variant of any one of embodiments 131-199, wherein [A][B][C] replaces positions 587-592 (e.g., A587, Q588, A589, Q590, A591, Q592), numbered according to SEQ ID NO: 138.
201. The AAV capsid variant of any one of embodiments 131-200, wherein [A][B][C] is present immediately subsequent to position 586 and replaces positions 587-592 (e.g., A587, Q588, A589, Q590, A591, Q592), numbered according to SEQ ID NO: 138.
202. The AAV capsid variant of any one of embodiments 131-201, wherein [A][B][C] corresponds to positions 587-599 of SEQ ID NO: 5, 8, or 3636.
203. The AAV capsid variant of any one of embodiments 147-202, wherein [D] is present immediately subsequent to position 592, relative to a reference sequence numbered according to the amino acid sequence of SEQ ID NO: 138.
204. The AAV capsid variant of any one of embodiments 147-203, wherein [D] replaces positions 593-595 (e.g., T593, G594, W595), numbered according to the amino acid sequence of SEQ ID NO: 138.
205. The AAV capsid variant of any one of embodiments 147-204, wherein [D] is present immediately subsequent to position 592, and replaces positions 593-595 (e.g., T593, G594, W595), numbered according to the amino acid sequence of SEQ ID NO: 138.
206. The AAV capsid variant of any one of embodiments 147-205, wherein [D] corresponds to positions 600-602 of SEQ ID NO: 5, 8, or 3636.
207. The AAV capsid variant of any one of embodiments 147-206, wherein [C][D] is present immediately subsequent to position 588, numbered according to SEQ ID NO: 138.
208. The AAV capsid variant of any one of embodiments 147-207, wherein [C][D] replaces positions 589-595 (e.g., A589, Q590, A591, Q592, T593, G594, W595), numbered according to the amino acid sequence of SEQ ID NO: 138.
209. The AAV capsid variant of any one of embodiments 147-208, wherein [C][D] is present immediately subsequent to 588, and replaces positions 589-595 (e.g., A589, Q590, A591, Q592, T593, G594, W595), numbered according to the amino acid sequence of SEQ ID NO: 138.
210. The AAV capsid variant of any one of embodiments 147-209, wherein [C][D] corresponds to positions 596-602 of SEQ ID NO: 5, 8, or 3636.
211. The AAV capsid variant of any one of embodiments 147-210, wherein [A][B][C][D] is present immediately subsequent to position 586, numbered according to SEQ ID NO: 5, 8, 138, or 3636.
212. The AAV capsid variant of any one of embodiments 147-211, wherein [A][B][C][D] replaces positions 587-595 (e.g., A587, Q588, A589, Q590, A591, Q592, T593, G594, W595), numbered according to SEQ ID NO: 138.
213. The AAV capsid variant of any one of embodiments 147-212, wherein [A][B][C][D] is present immediately subsequent to position 586 and replaces positions 587-595 (e.g., A587, Q588, A589, Q590, A591, Q592, T593, G594, W595), numbered according to SEQ ID NO: 138.
214. The AAV capsid variant of any one of embodiments 147-213, wherein [A][B][C][D] corresponds to positions 587-602 of SEQ ID NO: 5, 8, or 3636.
215. The AAV capsid variant of any one of embodiments 159-92, wherein [E] is present immediately subsequent to position 595, relative to a reference sequence numbered according to the amino acid sequence of SEQ ID NO: 138.
216. The AAV capsid variant of any one of embodiments 159-92, wherein [E] replaces positions 596-598 (e.g., V596, Q597, N598), numbered according to the amino acid sequence of SEQ ID NO: 138.
217. The AAV capsid variant of any one of embodiments 159-216, wherein [E] is present immediately subsequent to position 595, and replaces positions 596-598 (e.g., V596, Q597, N598), numbered according to the amino acid sequence of SEQ ID NO: 138.
218. The AAV capsid variant of any one of embodiments 159-217, wherein [E] corresponds to positions 603 to 605 of SEQ ID NO: 5, 8, or 3636.
219. The AAV capsid variant of any one of embodiments 159-218, wherein [A][B][C][D][E] is present immediately subsequent to position 586, relative to a reference sequence numbered according to the amino acid sequence of SEQ ID NO: 5, 8, 138, or 3636.
220. The AAV capsid variant of any one of embodiments 159-219, wherein [A][B][C][D][E] replaces positions 587-598 (e.g., A587, Q588, A589, Q590, A591, Q592, T593, G594, W595, V596, Q597, N598), numbered according to SEQ ID NO: 138.
221. The AAV capsid variant of any one of embodiments 159-220, wherein [A][B][C][D][E] is present immediately subsequent to position 586 and replaces positions 587-598 (e.g., A587, Q588, A589, Q590, A591, Q592, T593, G594, W595, V596, Q597, N598), numbered according to SEQ ID NO: 138.
222. The AAV capsid variant of any one of embodiments 159-221, wherein [A][B][C][D][E] corresponds to positions 587-605 of SEQ ID NO: 5, 8, or 3636.
223. The AAV capsid variant of any one of embodiments 119-222, which comprises from N-terminus to C-terminus, [A][B].
224. The AAV capsid variant of any one of embodiments 131-223, which comprises from N-terminus to C-terminus, [A][B][C].
225. The AAV capsid variant of any one of embodiments 147-224, which comprises from N-terminus to C-terminus, [A][B][C][D].
226. The AAV capsid variant of any one of embodiments 159-225, which comprises from N-terminus to C-terminus, [A][B][C][D][E].
227. An AAV capsid variant, comprising PLNGAVHLY (SEQ ID NO: 3648) and optionally wherein the AAV capsid variant further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138.
228. The AAV capsid variant of embodiment 227, which comprises:
   (i) the amino acid T, A, L, R, V, C, I, K, M, N, P, Q, or S at position 593 numbered according to SEQ ID NO: 138 or at position 600 numbered according to SEQ ID NO: 5, 8, or 3636;
   (ii) the amino acid G, M, S, A, Q, V, T, L, P, H, K, N, I, Y, or R at position 594 numbered according to SEQ ID NO: 138 or at position 601 numbered according to SEQ ID NO: 5, 8, or 3636; and/or
   (iii) the amino acid W, S, P, T, A, G, L, Q, H, N, R, K, V, E, F, M, C or Y at position 595 numbered according to SEQ ID NO: 138 or at position 602 numbered according to SEQ ID NO: 5, 8, or 3636.
229. An AAV capsid variant, comprising the amino acid sequence of PLNGAVHLY (SEQ ID NO: 3648); and which further comprises one, two, or all of:
   (i) the amino acid T, A, L, R, V, C, I, K, M, N, P, Q, or S at position 593 numbered according to SEQ ID NO: 138 or at position 600 numbered according to SEQ ID NO: 5, 8, or 3636;
   (ii) the amino acid G, M, S, A, Q, V, T, L, P, H, K, N, I, Y, or R at position 594 numbered according to SEQ ID NO: 138 or at position 601 numbered according to SEQ ID NO: 5, 8, or 3636; and/or
   (iii) the amino acid W, S, P, T, A, G, L, Q, H, N, R, K, V, E, F, M, C, or Y at position 595 numbered according to SEQ ID NO: 138 or at position 602 numbered according to SEQ ID NO: 5, 8, or 3636;
   optionally, provided that the amino acids at positions 593-595 numbered according to SEQ ID NO: 138 or at positions 600-602 numbered according to SEQ ID NO: 5, 8, or 3636, does not comprise the amino acid sequence of TGW.
230. The AAV capsid variant of any one of embodiments 227-229, which comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138 or at position 601 numbered according to SEQ ID NO: 5, 8, or 3636; and/or
   (iii) the amino acid P at position 595 numbered according to SEQ ID NO: 138 or at position 602 numbered according to SEQ ID NO: 5, 8, or 3636.
231. The AAV capsid variant of any one of embodiments 227-230, which comprises the amino acid sequence TMS, ASP, LGS, LSS, RST, TAA, TAG, TAL, TAS, TGT, TMA, TQP, TSA, TSP, TST, TVA, TVS, VMS, VSP, VSS, VTP, TGP, VAS, AAA, AAP, AGP, AGW, ALP, AMS, AQP, ATG, ATP, ATQ, ATS, AVG, CSP, CTP, IAA, IAG, IAS, IGG, IGS, IGW, ILG, IPP, IQP, IQS, ISG, ISP, ISS, ITP, KGW, KSS, LAA, LAG, LAH, LAN, LAP, LAQ, LAR, LAS, LAT, LGP, LGQ, LGT, LHP, LKS, LLP, LMA, LMG, LMP, LMQ, LMS, LNP, LPP, LQP, LQQ, LQR, LSA, LSG, LSH, LSK, LSN, LSP, LSQ, LSR, LST, LTA, LTN, LTP, LTS, LVG, MAP, NAL, NAQ, NAS, NLP, NMQ, NQP, NSP, NTP, NTQ, NTT, NVQ, PGW, QAP, QSP, RAA, RAG, RAQ, RAS, RGG, RGS, RGW, RIA, RIG, RIP, RLG, RLP, RLS, RLT, RML, RMS, RNS, RPG, RPP, RQG, RQL, RQP, RQQ, RQS, RSA, RSG, RSP, RSQ, RSS, RSV, RTA, RTG, RTL, RTP, RTQ, RTS, RTT, RVE, RYS, SAA, SAF, SAG, SAK, SAL, SAM, SAN, SAP, SAQ, SAV, SGP, SLP, SMA, SMG, SMQ, SMS, SPP, SQL, SQM, SQP, SQQ, SSL, SSM, SSP, SSR, SSS, STP, STQ, STS, SVA, SVG, SVQ, SVS, TAK, TAM, TAN, TAP, TAQ, TAT, TAV, TAW, TGA, TGC, TGF, TGG, TGL, TGM, TGQ, TGR, TGS, TGV, TGY, TKA, TKP, TKQ, TKS, TLA, TLG, TLP, TLQ, TLR, TLS, TMG, TMK, TMM, TMN, TMP, TMQ, TMT, TNA, TNG, TNQ, TNS, TPA, TPG, TPP, TPQ, TPR, TPS, TPT, TPW, TQA, TQH, TQK, TQL, TQM, TQN, TQQ, TQR, TQT, TQW, TRW, TSG, TSH, TSK, TSL, TSM, TSN, TSQ, TSS, TSV, TSW, TTA, TTG, TTK, TTP, TTQ, TTR, TTS, TTT, TVG, TVQ, TVW, TYA, TYS, VAA, VAG, VAK, VAN, VAQ, VAT, VGG, VGK, VGN, VGS, VHP, VKA, VKP, VKQ, VLG, VLP, VLS, VMA, VMP, VMQ, VNA, VNG, VNS, VPA, VPP, VPQ, VQA, VQG, VQN, VQP, VQQ, VQS, VQT, VRP, VSA, VSG, VSN, VSQ, VSR, VST, VTA, VTG, VTK, VTS, or VAP at positions 593-595 numbered according to SEQ ID NO: 138 or at positions 600-602 numbered according to SEQ ID NO: 5, 8, or 3636.
232. The AAV capsid variant of any one of embodiments 227-231, which comprises the amino acid sequence LSP at positions 593-595 numbered according to SEQ ID NO: 138, or at positions 600-602 numbered according to SEQ ID NO: 5, 8, or 3636.
233. The AAV capsid variant of any one of embodiments 227-232, which further comprises:
   (i) one, two, three, or all of an amino acid other than A at position 589 (e.g., D, S, or T), an amino acid other than Q at position 590 (e.g., K, H, L, P, or R), an amino acid other than A at position 591 (e.g., P or E), and/or an amino acid other than Q at position 592 (e.g., H, K, or P), numbered according to SEQ ID NO: 138;
   (ii) one, two, three, or all of an amino acid other than A at position 596 (e.g., D, S, or T), an amino acid other than Q at position 597 (e.g., K, H, L, P, or R), an amino acid other than A at position 598 (e.g., P or E), and/or an amino acid other than Q at position 599 (e.g., H, K, or P), numbered according to SEQ ID NO: 5, 8, or 3636.
234. The AAV capsid variant of any one of embodiments 227-233, wherein:
   (i) the amino acid A, D, S, or T at position 589 numbered according to SEQ ID NO: 138, or at position 596 numbered according to SEQ ID NO: 5, 8, or 3636;
   (ii) the amino acid Q, K, H, L, P, or R at position 590 numbered according to SEQ ID NO: 138, or at position 597 numbered according to SEQ ID NO: 5, 8, or 3636;
   (iii) the amino acid A, E, or P at position 591 numbered according to SEQ ID NO: 138, or at position 598 numbered according to SEQ ID NO: 5, 8, or 3636; and/or
   (iv) the amino acid Q, H, K, or P at position 592 numbered according to SEQ ID NO: 138, or at position 599 numbered according to SEQ ID NO: 5, 8, or 3636.
235. The AAV capsid variant of any one of embodiments 227-234, which comprise the amino acid sequence of:
   (i) AQAQ (SEQ ID NO: 4737), AHAQ (SEQ ID NO: 4742), AKAQ (SEQ ID NO: 4741), ALAQ (SEQ ID NO: 4749), APAQ (SEQ ID NO: 4745), AQAH (SEQ ID NO: 4747), AQAK (SEQ ID NO: 4746), AQAP (SEQ ID NO: 4743), AQEQ (SEQ ID NO: 4748), AQPQ (SEQ ID NO: 4739), ARAQ (SEQ ID NO: 4750), DQAQ (SEQ ID NO: 4744), SQAQ (SEQ ID NO: 4738), or TQAQ (SEQ ID NO: 4751) at positions 589-592 numbered according to SEQ ID NO: 138 or at positions 596-599 numbered according to SEQ ID NO: 5, 8, or 3636; or
   (ii) AQAQ (SEQ ID NO: 4737), AKAQ (SEQ ID NO: 4741), AQPQ (SEQ ID NO: 4739), DQAQ (SEQ ID NO: 4744), or SQAQ (SEQ ID NO: 4738) at positions 589-592 numbered according to SEQ ID NO: 138 or at positions 596-599 numbered according to SEQ ID NO: 5, 8, or 3636.
236. The AAV capsid variant of any one of embodiments 227-235, which further comprises:
   (i) one, two, or all of an amino acid other than V at position 596 (e.g., G, F, D, L, A, I, or E), an amino acid other than Q at position 597 (e.g., K, R, H, E, L, or P), and/or an amino acid other than N at position 598 (e.g., H, K, T, I, S, D, P, or Y), numbered according to SEQ ID NO: 138; or
   (ii) one, two, or all of an amino acid other than V at position 603 (e.g., G, F, D, L, A, I, or E), an amino acid other than Q at position 604 (e.g., K, R, H, E, L, or P), and/or an amino acid other than N at position 605 (e.g., H, K, T, I, S, D, P, or Y), numbered according to SEQ ID NO: 5, 8, or 3636.
237. The AAV capsid variant of any one of embodiments 227-237, wherein:
   (i) the amino acid V, G, F, D, L, A, I, or E at position 596 numbered according to SEQ ID NO: 138 or at position 603 numbered according to SEQ ID NO: 5, 8, or 3636;
   (ii) the amino acid Q, K, R, H, E, L, or P at position 597, numbered according to SEQ ID NO: 138 or at position 604 numbered according to SEQ ID NO: 5, 8, or 3636; and/or
   (iii) the amino acid N, H, K, T, I, S, D, P, or Y at position 598 numbered according to SEQ ID NO: 138 or at position 605 numbered according to SEQ ID NO: 5, 8, or 3636.
238. An AAV capsid variant, comprising PLNGAVHLY (SEQ ID NO: 3648), and optionally wherein the AAV capsid variant further comprises one, two, or all of an amino acid other than V at position 596 (e.g., G, F, D, L, A, I, or E), an amino acid other than Q at position 597 (e.g., K, R, H, E, L, or P), and/or an amino acid other than N at position 598 (e.g., H, K, T, I, S, D, P, or Y), numbered according to SEQ ID NO: 138.
239. An AAV capsid variant, comprising PLNGAVHLY (SEQ ID NO: 3648), and further comprising one, two, or all of:
   (i) V, G, F, D, L, A, I, or E at position 596 numbered according to SEQ ID NO: 138 or at position 603 numbered according to SEQ ID NO: 5, 8, or 3636;
   (ii) Q, K, R, H, E, L, or P at position 597 numbered according to SEQ ID NO: 138 or at position 604 numbered according to SEQ ID NO: 5, 8, or 3636; and/or
   (iii) N, H, K, T, I, S, D, P, or Y at position 598 numbered according to SEQ ID NO: 138 or at position 605 numbered according to SEQ ID NO: 5, 8, or 3636;
   optionally, provided that the amino acids at positions 596-598 numbered according to the amino acid sequence of SEQ ID NO: 138 or positions 603-605 of SEQ ID NO: 5, 8, or 3636, does not comprise the amino acid sequence of VQN.
240. The AAV capsid variant of any one of embodiments 227-239, which comprises the amino acid P at position 597 numbered according to SEQ ID NO: 138 or at position 604 numbered according to SEQ ID NO: 5, 8, or 3636.
241. The AAV capsid variant of any one of embodiments 227-339, which comprises the amino acid K at position 597 numbered according to SEQ ID NO: 138 or at position 604 numbered according to SEQ ID NO: 5, 8, or 3636.
242. The AAV capsid variant of any one of embodiments 227-241, which comprises the amino acid sequence of:
   (i) GQN, VQH, VQK, VQT, VQN, FQN, VKN, VQI, DQN, LQN, VQS, VRN, AQN, IQN, VHN, VLN, VEN, VQD, DKN, EQN, FQK, GHN, GQT, VKK, VKT, VPH, VPN, VQP, or VQY at positions 596-598 numbered according to SEQ ID NO: 138 or positions 603-605 numbered according to SEQ ID NO: 5, 8, or 3636; or
   (ii) VQN, VQT, VQK, DQN, VQH, FQN, AQN, VLN, LQN, VQI, VQS, EQN, GQT, VPN, VQD, VQP, VRN, or VKN at positions 596-598 numbered according to SEQ ID NO: 138 or positions 603-605 numbered according to SEQ ID NO: 5, 8, or 3636.
243. The AAV capsid variant of any one of embodiments 227-242, which comprises the amino acid sequence of VKN, VPN, or VQN at positions 596-598 numbered according to SEQ ID NO: 138 or positions 603-605 numbered according to SEQ ID NO: 5, 8, or 3636.
244. The AAV capsid variant of any one of embodiments 227-242, which comprises the amino acid sequence of VEN or VHN at positions 596-598 numbered according to SEQ ID NO: 138 or positions 603-605 numbered according to SEQ ID NO: 5, 8, or 363.
245. The AAV capsid variant of any one of embodiments 227-244, which comprises:
   (i) the amino acid sequence of any of SEQ ID NOs: 143, 148, 149, 151, 153-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284, 285, 286, 288-293, 295, 296, 298, 300-314, 316-318, 320-327, 329, 331, 332, 334, 336-344, 346-350, 352-354, 356-367, 369, 371-380, 382-385, 387, 392-394, 396, 397, 399-401, 404-411, 413-415, 417, 419-429, 432, 433, 435-437, 438, 440-442, 444-447, 450-453, 456, 458-461, 464, 465, 467-469, 471-478, 480-483, 487-495, 497, 498, 500-503, 505, 507-512, 515-517, 522-525, 528-532, 534-539, 542-545, 547, 551-554, 558-561, 563-568, 570, 573, 574, 576, 579, 581, 582, 584, 586, 587, 592-596, 598, 601, 604-607, 610, 612, 614-619, 624-629, 631, 633-636, 640, 641, 645, 646, 649, 650, 658, 663, 664, 666, 668, 669, 672, 673, 675, 679, 683, 684, 686, 688, 689, 691, 693, 695, 697, 699, 700, 704, 705, 709-712, 720, 722, 726-731, 733, 736, 740, 745, 749, 750-752, 754, 755, 757, 758, 760-765, 767, 768, 771, 778, 780, 783-787, 792, 794, 797, 799-802, 804, 817, 818, 821, 824, 828, 831, 832, 834-837, 840-845, 847, 848, 851-853, 855, 858, 861, 862, 865, 869, 870-872, 874, 876, 882, 883, 887, 889, 890, 892-895, 897, 901, 903, 904, 905, 907, 910, 911, 913, 915, 919, 920, 923, 924, 926, 927, 929, 931-933, 935, 937, 940, 941, 943, 945-949, 953, 955, 957, 958, 960, 962, 964, 965, 971, 973, 974, 977, 986, 988, 989, 992, 994, 997, 998, 1000, 1004, 1007, 1013, 1015, 1017, 1018, 1020, 1025, 1027, 1029, 1030, 1031, 1033-1035, 1037-1039, 1043, 1046, 1049, 1052, 1056, 1057, 1059, 1062, 1065, 1067, 1068, 1070, 1073, 1075, 1077-1079, 1083-1087, 1089, 1090, 1094, 1100, 1101, 1103, 1106, 1107, 1110, 1111, 1112, 1114, 1115, 1117, 1119, 1125, 1126, 1129, 1132, or 1133;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
246. The AAV capsid variant of any one of embodiments 227-245, which comprises:
   (i) the amino acid sequence of any of SEQ ID NOs: 143, 148-151, 153-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-318, 320-327, 329, 331, 332, 334, 336, 337-339, 340-344, 346-350, 352-354, 356-365, 367, 369, 371-380, 382-385, 387, 392-394, 396, 397, 399-401, 404-411, 413-415, 417, 419, 420-429, 432, 433, 435-438, 440-442, 444-447, 450-453, 456, 458, 459, 460, 461, 464, 465, 467-469, or 471-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
247. The AAV capsid variant of any one of embodiments 227-246, wherein the amino acid sequence of PLNGAVHLY (SEQ ID NO: 3648) is present in loop VIII.
248. The AAV capsid variant of any one of embodiments 227-247, wherein the amino acid sequence of PLNGAVHLY (SEQ ID NO: 3648) is present immediately subsequent to position 586, relative to a reference sequence numbered according to SEQ ID NO: 5, 8, 138, or 3636.
249. The AAV capsid variant of any one of embodiments 227-248, wherein the amino acid sequence of PLNGAVHLY (SEQ ID NO: 3648) replaces positions 587 and 588 (e.g., A587 and Q588), numbered according to SEQ ID NO: 138.
250. The AAV capsid variant of any one of embodiments 227-249, wherein the amino acid sequence of PLNGAVHLY (SEQ ID NO: 3648) is present immediately subsequent to position 586 and replaces positions 587 and 588 (e.g., A587 and Q588), numbered according to SEQ ID NO: 138.
251. The AAV capsid variant of any one of embodiments 227-111, wherein the amino acid sequence of PLNGAVHLY (SEQ ID NO: 3648) corresponds to positions 587-595 of SEQ ID NO: 5, 8, or 3636.
252. An AAV capsid variant comprising X1-X2-X3-X4-X5-X6-X7-X8-X9-X10-X11-X12-X13-X14-X15-X16-X17-X18-X19, wherein:
   (i) X1 is: P, A, D, E, F, G, H, K, L, N, Q, R, S, T, or V;
   (ii) X2 is: L, D, E, F, H, I, M, N, P, Q, R, S, or V;
   (iii) X3 is: N, A, D, E, G, H, I, K, Q, S, T, V, or Y;
   (iv) X4 is: G, A, C, D, E, P, Q, R, S, T, V, or W;
   (v) X5 is: A, C, D, E, F, G, H, I, K, N, P, Q, R, S, T, V, W, or Y;
   (vi) X6 is: V, A, C, D, E, F, G, H, I, K, L, M, N, Q, R, S, T, or Y;
   (vii) X7 is: H, A, D, E, G, I, K, L, M, N, P, Q, R, S, T, V, or Y;
   (viii) X8 is: L, A, D, E, F, G, H, I, K, M, N, P, Q, R, S, T, V, or Y;
   (ix) X9 is: Y, A, C, D, E, F, G, H, I, K, L, M, N, Q, R, S, T, V, or W;
   (x) X10 is: A, C, D, E, F, G, H, I, K, L, N, P, Q, R, S, T, V, or; Y;
   (xi) X11 is: Q, A, D, E, H, K, L, P, R, or T;
   (xii) X12 is: A, D, E, G, H, L, N, P, Q, R, S, T, or V;
   (xiii) X13 is: Q, E, H, K, L, P, R, or T;
   (xiv) X14 is: T, A, C, D, E, F, G, H, I, K, L, M, N, P, Q, R, S, V, W, or Y;
   (xv) X15 is: G, A, C, D, E, F, H, I, K, L, M, N, P, Q, R, S, T, V, W, or Y;
   (xvi) X16 is: W, A, C, D, E, F, G, H, I, K, L, M, N, P, Q, R, S, T, V, or Y;
   (xvii) X17 is: V, A, D, E, F, G, H, I, or L;
   (xviii) X18 is: Q, E, H, K, L, P, or R; and/or
   (xix) X19 is: N, D, H, I, K, P, S, T, or Y.
253. The AAV capsid variant of embodiment 252, wherein:
   (i) X1 is: P, Q, A, S, T, R, H, L, or K;
   (ii) X2 is: L, I, V, H, or R;
   (iii) X3 is: N, D, K, Y, or I;
   (iv) X4 is: G, S, R, C, or A;
   (v) X5 is: A, S, G, N, T, D, Y, Q, V, or C;
   (vi) X6 is: V, I, L, A, F, D, or G;
   (vii) X7 is: H, N, Q, P, D, L, R, or Y;
   (viii) X8 is: L, H, V, I, or R;
   (ix) X9 is Y;
   (x) X10 is: A, D, S, or T;
   (xi) X11 is: Q, K, H, L, P, or R;
   (xii) X12 is: A, P, E, or S;
   (xiii) X13 is: Q, K, H, or P;
   (xiv) X14 is: L, T, V, S, R, I, A, N, C, P, Q, M, or K;
   (xv) X15 is: S, G, M, T, A, K, Q, V, I, R, N, P, L, H, Y;
   (xvi) X16 is: P, W, S, K, Q, G, C, R, A, N, T, V, M, H, L, E, F, or Y;
   (xvii) X17 is: V, D, F, A, E, L, G, or I;
   (xviii) X18 is: Q, R, P, K, L, H, or E; and/or
   (xix) X19 is: N, H, D, S, T, P, K, I, or Y.
254. The AAV capsid variant of embodiment 252, wherein:
   (i) X1 is: P, A, S, Q, or T;
   (ii) X2 is L or I;
   (iii) X3 is N or D;
   (iv) X4 is G or S;
   (v) X5 is: A, S, G, N, or T;
   (vi) X6 is V;
   (vii) X7 is H;
   (viii) X8 is: L, H, V, or I
   (ix) X9 is Y;
   (x) X10 is: A, D, or S;
   (xi) X11 is Q or K;
   (xii) X12 is A or P;
   (xiii) X13 is Q;
   (xiv) X14 is: L, T, V, S, R, I, A, N, C, P, Q, or M;
   (xv) X15 is: S, G, M, T, A, K, Q, V, I, R, N, P, L, or H;
   (xvi) X16 is: P, W, S, K, Q, G, C, R, A, N, T, V, M, H, L, or E;
   (xvii) X17 is: V, D, F, A, E, L, or G;
   (xviii) X18 is: Q, R, P, K, or L; and/or
   (xix) X19 is: N, H, D, S, T, P, K, or I.
255. The AAV capsid variant of any one of embodiments 252-254, wherein X1-X2-X3-X4-X5-X6-X7-X8-X9-X10-X11-X12-X13-X14-X15-X16-X17-X18-X19 is present immediately subsequent to position 586, numbered according to SEQ ID NO: 5, 8, 138, or 3636.
256. The AAV capsid variant of any one of embodiments 252-255, wherein X1-X2-X3-X4-X5-X6-X7-X8-X9-X10-X11-X12-X13-X14-X15-X16-X17-X18-X19 replaces positions 587-598 (e.g., A587, Q588, A589, Q590, A591, Q592, T593, G594, W595, V596, Q597, N598), numbered according to SEQ ID NO: 138.
257. The AAV capsid variant of any one of embodiments 252-256, wherein X1-X2-X3-X4-X5-X6-X7-X8-X9-X10-X11-X12-X13-X14-X15-X16-X17-X18-X19 is present immediately subsequent to position 586 and replaces positions 587-598 (e.g., A587, Q588, A589, Q590, A591, Q592, T593, G594, W595, V596, Q597, N598), numbered according to SEQ ID NO: 138.
258. The AAV capsid variant of any one of embodiments 252-257, wherein X1-X2-X3-X4-X5-X6-X7-X8-X9-X10-X11-X12-X13-X14-X15-X16-X17-X18-X19 corresponds to positions 587-605 of SEQ ID NO: 5, 8, or 3636.
259. An AAV capsid variant comprising an amino sequence comprising the following formula: [N1]-[N2], wherein:
   (i) [N1] comprises the amino acid sequence of PLNG (SEQ ID NO: 3678); and
   (ii) [N2] comprises X1, X2, and X3, wherein:
      (a) position X1 is: A, V, T, or G;
      (b) position X2 is: R, K, Q, G, or V; and
      (c) position X3 is: H, A, M, S, T, Q, or Y, or; and/or
   an amino acid modification, e.g., a conservative substitution, of any of the aforesaid amino acids in (i) and/or (ii).
260. An AAV capsid variant comprising one, two, or all of:
   (i) an [N1], wherein [N1] is or comprises: PLNN (SEQ ID NO: 4752), PLNG (SEQ ID NO: 3678), PSAR (SEQ ID NO: 4753), TLNG (SEQ ID NO: 4754), PLNM (SEQ ID NO: 4755), SLNG (SEQ ID NO: 4756), SING (SEQ ID NO: 4757), ALNG (SEQ ID NO: 4758), PLNL (SEQ ID NO: 4759), PGRQ (SEQ ID NO: 4760), or LVNS (SEQ ID NO: 4761);
   (ii) an [N2] wherein [N2] is or comprises: PGH, VKA, ARM, VKM, VRA, VRS, TRM, VRT, VRM, AKM, VKS, VQM, AVH, TRS, VRQ, AQM, VKY, ART, AGA, VQA, VKT, PVH, GVH, AGH, VGH, TGH, or TVR; and/or
   (iii) an [N3] wherein [N3] is or comprises: LY, IY, LN, DY, LS, or VS; and/or wherein the AAV capsid variant comprises an amino acid modification, e.g., a conservative substitution, of any of the aforesaid amino acids in (i)-(v).
261. The AAV capsid variant of embodiment 259, wherein [N2] comprises VK, AR, VR, TR, AK, VQ, AQ, AG, GV, KA, RM, KM, RA, RS, RT, KS, QM, RQ, KY, GA, QA, KT, VH, or GH.
262. The AAV capsid variant of any one of embodiments 259-261, wherein [N2] is or comprises VKA, ARM, VKM, VRA, VRS, TRM, VRT, VRM, AKM, VKS, VQM, TRS, VRQ, AQM, VKY, ART, AGA, VQA, VKT, GVH, AGH.
263. The AAV capsid variant of any one of embodiments 259, 261, or 262, wherein [N1]-[N2] comprises:

(i)
(SEQ ID NO: 5438)
PLNGVK,
(SEQ ID NO: 5439)
PLNGAR,
(SEQ ID NO: 5440)
PLNGVR,
(SEQ ID NO: 5441)
PLNGTR,
(SEQ ID NO: 5442)
PLNGAK,
(SEQ ID NO: 5443)
PLNGVQ,
(SEQ ID NO: 5444)
PLNGAQ,
(SEQ ID NO: 5445)
PLNGAG,
or
(SEQ ID NO: 5446)
PLNGGV;

(ii) an amino acid sequence comprising any portion of an amino acid sequence in (i), e.g., any 2, 3, 4, or 5 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
264. The AAV capsid variant of any one of embodiments 259-263, wherein [N1]-[N2] is or comprises:

(i)
(SEQ ID NO: 5447)
PLNGVKA,
(SEQ ID NO: 5448)
PLNGARM,
(SEQ ID NO: 5449)
PLNGVKM,
(SEQ ID NO: 5450)
PLNGVRA,
(SEQ ID NO: 5451)
PLNGVRS,
(SEQ ID NO: 5452)
PLNGTRM,
(SEQ ID NO: 5453)
PLNGVRT,
(SEQ ID NO: 5454)
PLNGVRM,
(SEQ ID NO: 5455)
PLNGAKM,
(SEQ ID NO: 5456)
PLNGVKS,
(SEQ ID NO: 5457)
PLNGVQM,
(SEQ ID NO: 5458)
PLNGTRS,
(SEQ ID NO: 5459)
PLNGVRQ,
(SEQ ID NO: 5460)
PLNGAQM,
(SEQ ID NO: 5461)
PLNGVKY,
(SEQ ID NO: 5462)
PLNGART,
(SEQ ID NO: 5463)
PLNGAGA,
(SEQ ID NO: 5464)
PLNGVQA,
(SEQ ID NO: 5465)
PLNGVKT,
(SEQ ID NO: 5466)
PLNGGVH,
(SEQ ID NO: 5120)
PLNGAGH;

(ii) an amino acid sequence comprising any portion of an amino acid sequence in (i), e.g., any 2, 3, 4, 5, or 6 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
265. The AAV capsid variant of any one of embodiments 259-264, which further comprises [N3], wherein [N3] comprises X4 and X5, wherein:
   (a) position X4 is: L, I, or D; and
   (b) position X5 is: Y, S, or N; and/or
an amino acid modification, e.g., a conservative substitution, of any of the aforesaid amino acids in (a) or (b).
266. The capsid variant of embodiment 260 or 265, wherein [N3] is or comprises LY, IY, DY, LS, or LN.
267. The AAV capsid variant of embodiment 265 or 266, wherein [N2]-[N3] comprises: KAL, RMI, KML, RAL, RSL, RML, RTL, RML, RML, KML, KSL, QML, RSL, RQL, QML, KYL, RTI, GAD, QAL, KTL, VHL, GHL.
268. The AAV capsid variant of any one of embodiments 265-267, wherein [N2]-[N3] comprises:

(i)
(SEQ ID NO: 5467)
VKAL,
(SEQ ID NO: 5468)
ARMI,
(SEQ ID NO: 5469)
VKML,
(SEQ ID NO: 5470)
VRAL,
(SEQ ID NO: 5471)
VRSL,
(SEQ ID NO: 5472)
TRML,
(SEQ ID NO: 5473)
VRTL,
(SEQ ID NO: 5474)
VRML,
(SEQ ID NO: 5475)
ARML,
(SEQ ID NO: 5476)
AKML,
(SEQ ID NO: 5477)
VKSL,
(SEQ ID NO: 5478)
VQML,
(SEQ ID NO: 5479)
TRSL,
(SEQ ID NO: 5480)
VRQL,
(SEQ ID NO: 5481)
AQML,
(SEQ ID NO: 5482)
VKYL,
(SEQ ID NO: 5483)
ARTI,
(SEQ ID NO: 5484)
AGAD,
(SEQ ID NO: 5485)
VQAL,
(SEQ ID NO: 5486)
VKTL,
(SEQ ID NO: 5487)
GVHL,
or
(SEQ ID NO: 5488)
AGHL;

(ii) an amino acid sequence comprising any portion of an amino acid sequence in (i), e.g., any 2 or 3 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
269. The AAV capsid variant of any one of embodiments 260 or 265-268, wherein [N2]-[N3] is or comprises:

(i)
(SEQ ID NO: 5489)
VKALY,
(SEQ ID NO: 5490)
ARMIY,
(SEQ ID NO: 5491)
VKMLY,
(SEQ ID NO: 5492)
VRALY,
(SEQ ID NO: 5493)
VRSLY,
(SEQ ID NO: 5494)
TRMLY,
(SEQ ID NO: 5495)
VRTLY,
(SEQ ID NO: 5496)
VRMLY,
(SEQ ID NO: 5497)
ARMLY,
(SEQ ID NO: 5498)
AKMLY,
(SEQ ID NO: 5499)
VKSLY,
(SEQ ID NO: 5500)
VQMLY,
(SEQ ID NO: 5501)
TRSLY,
(SEQ ID NO: 5502)
VRQLY,
(SEQ ID NO: 5503)
AQMLY,
(SEQ ID NO: 5504)
VKYLY,
(SEQ ID NO: 5505)
ARTIY,
(SEQ ID NO: 5506)
AGADY,
(SEQ ID NO: 5507)
VQALY,
(SEQ ID NO: 5508)
VKTLY,
(SEQ ID NO: 5509)
GVHLS,
or
(SEQ ID NO: 5510)
AGHLN;

(ii) an amino acid sequence comprising any portion of an amino acid sequence in (i), e.g., any 2, 3, or 4 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
270. The AAV capsid variant of any one of embodiments 265-269, wherein [N1]-[N2]-[N3] comprises:

(i)
(SEQ ID NO: 5511)
PLNGVKAL,
(SEQ ID NO: 5512)
PLNGARMI,
(SEQ ID NO: 5513)
PLNGVKML,
(SEQ ID NO: 5514)
PLNGVRAL,
(SEQ ID NO: 5515)
PLNGVRSL,
(SEQ ID NO: 5516)
PLNGTRML,
(SEQ ID NO: 5517)
PLNGVRTL,
(SEQ ID NO: 5518)
PLNGVRML,
(SEQ ID NO: 5519)
PLNGARML,
(SEQ ID NO: 5520)
PLNGAKML,
(SEQ ID NO: 5521)
PLNGVKSL,
(SEQ ID NO: 5522)
PLNGVQML,
(SEQ ID NO: 5523)
PLNGTRSL,
(SEQ ID NO: 5524)
PLNGVRQL,
(SEQ ID NO: 5525)
PLNGAQML,
(SEQ ID NO: 5526)
PLNGVKYL,
(SEQ ID NO: 5527)
PLNGARTI,
(SEQ ID NO: 5528)
PLNGAGAD,
(SEQ ID NO: 5529)
PLNGVQAL,
(SEQ ID NO: 5530)
PLNGVKTL,
(SEQ ID NO: 5531)
PLNGGVHL,
or
(SEQ ID NO: 5532)
PLNGAGHL;

(ii) an amino acid sequence comprising any portion of an amino acid sequence in (i), e.g., any 2, 3, 4, 5, 6, or 7 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
271. The AAV capsid variant of any one of embodiments 260 or 265-270, wherein [N1]-[N2]-[N3] is or comprises:

(i)
(SEQ ID NO: 5533)
PLNGVKALY,
(SEQ ID NO: 5534)
PLNGARMIY,
(SEQ ID NO: 5535)
PLNGVKMLY,
(SEQ ID NO: 5536)
PLNGVRALY,
(SEQ ID NO: 5537)
PLNGVRSLY,
(SEQ ID NO: 5538)
PLNGTRMLY,
(SEQ ID NO: 5539)
PLNGVRTLY,
(SEQ ID NO: 5540)
PLNGVRMLY,
(SEQ ID NO: 5541)
PLNGARMLY,
(SEQ ID NO: 5542)
PLNGAKMLY,
(SEQ ID NO: 5543)
PLNGVKSLY,
(SEQ ID NO: 5544)
PLNGVQMLY,
(SEQ ID NO: 5545)
PLNGTRSLY,
(SEQ ID NO: 5546)
PLNGVRQLY,
(SEQ ID NO: 5547)
PLNGAQMLY,
(SEQ ID NO: 5548)
PLNGVKYLY,
(SEQ ID NO: 5549)
PLNGARTIY,
(SEQ ID NO: 5550)
PLNGAGADY,
(SEQ ID NO: 5551)
PLNGVQALY,
(SEQ ID NO: 5552)
PLNGVKTLY,
(SEQ ID NO: 5553)
PLNGGVHLS,
or
(SEQ ID NO: 5554)
PLNGAGHLN;

(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
272. The AAV capsid variant of any one of embodiments 260 or 265-271, wherein:
   (i) [N1] is or comprises: PLNG (SEQ ID NO: 3678);
   (ii) [N2] is or comprises: VKA, ARM, VKM, VRA, VRS, TRM, VRT, VRM, AKM, VKS, VQM, TRS, VRQ, AQM, VKY, ART, AGA, VQA, VKT, GVH, AGH; and/or
   (iii) [N3] is or comprises: LY, IY, DY, LS, or LN.
273. The AAV capsid variant of any one of embodiments 259-272, wherein [N1], [N2], and/or [N3] is present in loop VIII of the AAV capsid variant.
274. The AAV capsid variant of any one of embodiments 259-129, wherein [N1] is present immediately subsequent to position 586, numbered according to SEQ ID NO: 5, 8, 138, or 3636.
275. The AAV capsid variant of any one of embodiments 259-274, wherein [N1] replaces positions 587 and 588 (e.g., A 587 and Q588), numbered according to SEQ ID NO: 138.
276. The AAV capsid variant of any one of embodiments 259-275, wherein [N1] is present immediately subsequent to position 586 and replaces positions 587 and 588 (e.g., A 587 and Q588), numbered according to SEQ ID NO: 138.
277. The AAV capsid variant of any one of embodiments 259-276, wherein [N1] corresponds to positions 587-590 of SEQ ID NO: 5, 8, or 3636.
278. The AAV capsid variant of any one of embodiments 259-27, wherein [N2] is present immediately subsequent to [N1].
279. The AAV capsid variant of any one of embodiments 259-278, wherein [N2] corresponds to positions 591-593 of SEQ ID NO: 5, 8, or 3636.
280. AAV capsid variant of any one of embodiments 259-279, [N1]-[N2] is present immediately subsequent to position 586, numbered according to SEQ ID NO: 138.
281. The AAV capsid variant of any one of embodiments 259-280, wherein [N1]-[N2] replaces positions 587 and 588 (e.g., A 587 and Q588), numbered according to SEQ ID NO: 138.
282. The AAV capsid variant of any one of embodiments 259-281, wherein [N1]-[N2] is present immediately subsequent to position 586 and replaces positions 587 and 588 (e.g., A 587 and Q588), numbered according to SEQ ID NO: 138.
283. The AAV capsid variant of any one of embodiments 259-282, wherein [N1]-[N2] corresponds to positions 587-593 of SEQ ID NO: 5, 8, or 3636.
284. The AAV capsid variants of any one of embodiments 260 or 265-283, wherein [N3] is present immediately subsequent to [N2].
285. The AAV capsid variants of any one of embodiments 260 or 265-284, wherein [N3] corresponds to positions 594 and 595 of SEQ ID NO: 5, 8, or 3636.
286. The AAV capsid variants of any one of embodiments 260 or 265-285, wherein [N2]-[N3] is present immediately subsequent to [N1].
287. The AAV capsid variants of any one of embodiments 260 or 265-286, wherein [N1]-[N2]-[N3] is present immediately subsequent to position 586, numbered according to SEQ ID NO: 138.
288. The AAV capsid variants of any one of embodiments 260 or 265-287, wherein [N1]-[N2]-[N3] replaces positions 587 and 588 (e.g., A 587 and Q588), numbered according to SEQ ID NO: 138.
289. The AAV capsid variants of any one of embodiments 260 or 265-288, wherein [N1]-[N2]-[N3] is present immediately subsequent to position 586 and replaces positions 587 and 588 (e.g., A 587 and Q588), numbered according to SEQ ID NO: 138.
290. The AAV capsid variants of any one of embodiments 260 or 265-289, wherein [N1]-[N2]-[N3] corresponds to positions 587-595 of SEQ ID NO: 5, 8, or 3636.
291. An AAV capsid variant, comprising an amino sequence comprising the following formula: [B]-[C], wherein:
   (i) [B] comprises X1, X2, and X3, wherein:
      (a) position X1 is: P, V, A, or T;
      (b) position X2 is: G, K, R, Q, or V; and
      (c) position X3 is: H, A, M, S, T, Q, or Y; and
   (ii) [C] comprises the amino acid sequence of LY.
292. An AAV capsid variant comprising one, two, or all of:
   (i) an [A], wherein [A] is or comprises: PLNN (SEQ ID NO: 4752), PLNG (SEQ ID NO: 3678), PSAR (SEQ ID NO: 4753), PLNM (SEQ ID NO: 4755), SLNG (SEQ ID NO: 4756), SING (SEQ ID NO: 4757), PLNL (SEQ ID NO: 4759), or PGRQ (SEQ ID NO: 4760);
   (ii) a [B], wherein [B] is or comprises: PGH, VKA, VKM, VRA, VRS, TRM, VRT, VRM, ARM, AKM, VKS, VQM, AVH, TRS, VRQ, AQM, VKY, VQA, VKT, PVH, VGH, or TGH; and/or
   (iii) a [C] wherein [C] is or comprises: LY.
293. The AAV capsid variant of embodiment 291, wherein [B] comprises PG, VK, VR, TR, AR, AK, VQ, AV, AQ, PV, VG, TG, GH, KA, KM, RA, RS, RM, RT, KS, QM, VH, RQ, KY, QA, or KT.
294. The AAV capsid variant of any one of embodiments 291-293, wherein [B] is or comprises PGH, VKA, VKM, VRA, VRS, TRM, VRT, VRM, ARM, AKM, VKS, VQM, AVH, TRS, VRQ, AQM, VKY, VQA, VKT, PVH, VGH, or TGH.
295. The AAV capsid variant of any one of embodiments 291, 293, or 294, wherein [B]-[C] comprises:

(i)
(SEQ ID NO: 4734)
GHLY,
(SEQ ID NO: 5555)
KALY,
(SEQ ID NO: 5556)
KMLY,
(SEQ ID NO: 5557)
RALY,
(SEQ ID NO: 5558)
RSLY,
(SEQ ID NO: 5559)
RMLY,
(SEQ ID NO: 5560)
RTLY,
(SEQ ID NO: 5559)
RMLY,
(SEQ ID NO: 5559)
RMLY,
(SEQ ID NO: 5556)
KMLY,
(SEQ ID NO: 5561)
KSLY,
(SEQ ID NO: 5562)
QMLY,
(SEQ ID NO: 4680)
VHLY,
(SEQ ID NO: 5558)
RSLY,
(SEQ ID NO: 5563)
RQLY,
(SEQ ID NO: 5562)
QMLY,
(SEQ ID NO: 5564)
KYLY,
(SEQ ID NO: 4734)
GHLY,
(SEQ ID NO: 5565)
QALY,
(SEQ ID NO: 4680)
VHLY,
(SEQ ID NO: 5566)
KTLY,
(SEQ ID NO: 4680)
VHLY,
(SEQ ID NO: 4734)
GHLY,
(SEQ ID NO: 4734)
GHLY,
or
(SEQ ID NO: 4680)
VHLY;

(ii) an amino acid sequence comprising any portion of an amino acid sequence in (i, e.g., any 2, or 3 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
296. The AAV capsid variant of any one of embodiments 291-295, wherein [B]-[C] is or comprises:

(i)
(SEQ ID NO: 5567)
PGHLY,
(SEQ ID NO: 5489)
VKALY,
(SEQ ID NO: 5491)
VKMLY,
(SEQ ID NO: 5492)
VRALY,
(SEQ ID NO: 5493)
VRSLY,
(SEQ ID NO: 5494)
TRMLY,
(SEQ ID NO: 5495)
VRTLY,
(SEQ ID NO: 5496)
VRMLY,
(SEQ ID NO: 5497)
ARMLY,
(SEQ ID NO: 5498)
AKMLY,
(SEQ ID NO: 5499)
VKSLY,
(SEQ ID NO: 5500)
VQMLY,
(SEQ ID NO: 3687)
AVHLY,
(SEQ ID NO: 5501)
TRSLY,
(SEQ ID NO: 5502)
VRQLY,
(SEQ ID NO: 5503)
AQMLY,
(SEQ ID NO: 5504)
VKYLY,
(SEQ ID NO: 5507)
VQALY,
(SEQ ID NO: 5508)
VKTLY,
(SEQ ID NO: 5568)
PVHLY,
(SEQ ID NO: 5569)
VGHLY,
or
(SEQ ID NO: 5570)
TGHLY;

(ii) an amino acid sequence comprising any portion of an amino acid sequence in (i), e.g., any 2, or 3 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
297. The AAV capsid of any one of embodiments 291-296, which further comprises [A], wherein [A] comprises X4, X5, X6, and X7, wherein:
   (a) position X4 is P or S;
   (b) position X5 is L, S, I, or G;
   (c) position X6 is N, A, or R; and
   (d) position X7 is N, G, R, M, L, or Q.
298. The AAV capsid variant of embodiment 297, wherein [A] comprises PL, PS, SL, SI, PG, NN, NG, AR, NM, NL, RQ, LN, SA, IN, or GR.
299. The AAV capsid variant of embodiment 297 or 298, wherein [A] comprises PLN, PSA, SLN, SIN, PGR, LNN, LNG, SAR, LNM, ING, LNL, or GRQ.
300. The AAV capsid variant of embodiment 292 or 297-298, wherein [A] is or comprises PLNN (SEQ ID NO: 4752), PLNG (SEQ ID NO: 3678), PSAR (SEQ ID NO: 4753), PLNM (SEQ ID NO: 4755), SLNG (SEQ ID NO: 4756), SING (SEQ ID NO: 4757), PLNL (SEQ ID NO: 4759), or PGRQ (SEQ ID NO: 4760).
301. The AAV capsid variant of embodiment 297-300, wherein [A]-[B]-[C] comprises:

(i)
(SEQ ID NO: 5571)
NNPGHLY,
(SEQ ID NO: 5572)
NGVKALY,
(SEQ ID NO: 5573)
NGVKMLY,
(SEQ ID NO: 5574)
NGVRALY,
(SEQ ID NO: 5575)
NGVRSLY,
(SEQ ID NO: 5576)
NGTRMLY,
(SEQ ID NO: 5577)
NGVRTLY,
(SEQ ID NO: 5578)
NGVRMLY,
(SEQ ID NO: 5579)
NGARMLY,
(SEQ ID NO: 5580)
NGAKMLY,
(SEQ ID NO: 5581)
NGVKSLY,
(SEQ ID NO: 5582)
NGVQMLY,
(SEQ ID NO: 5583)
ARAVHLY,
(SEQ ID NO: 5584)
NGTRSLY,
(SEQ ID NO: 5585)
NGVRQLY,
(SEQ ID NO: 5586)
NGAQMLY,
(SEQ ID NO: 5587)
NGVKYLY,
(SEQ ID NO: 5588)
NMPGHLY,
(SEQ ID NO: 5589)
NGVQALY,
(SEQ ID NO: 3689)
NGAVHLY,
(SEQ ID NO: 5590)
NGVKTLY,
(SEQ ID NO: 5591)
NGPVHLY,
(SEQ ID NO: 5592)
NLVGHLY,
(SEQ ID NO: 5593)
NLTGHLY,
or
(SEQ ID NO: 5594)
RQAVHLY;

(ii) an amino acid sequence comprising any portion of an amino acid sequence in (i), e.g., any 2, 3, 4, 5, or 6 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
302. The AAV capsid variant of embodiment 292 or 297-301, wherein [A]-[B]-[C] is or comprises:

(i)
(SEQ ID NO: 5595)
PLNNPGHLY,
(SEQ ID NO: 5533)
PLNGVKALY,
(SEQ ID NO: 5535)
PLNGVKMLY,
(SEQ ID NO: 5536)
PLNGVRALY,
(SEQ ID NO: 5537)
PLNGVRSLY,
(SEQ ID NO: 5538)
PLNGTRMLY,
(SEQ ID NO: 5539)
PLNGVRTLY,
(SEQ ID NO: 5540)
PLNGVRMLY,
(SEQ ID NO: 5541)
PLNGARMLY,
(SEQ ID NO: 5542)
PLNGAKMLY,
(SEQ ID NO: 5543)
PLNGVKSLY,
(SEQ ID NO: 5544)
PLNGVQMLY,
(SEQ ID NO: 5596)
PSARAVHLY,
(SEQ ID NO: 5545)
PLNGTRSLY,
(SEQ ID NO: 5546)
PLNGVRQLY,
(SEQ ID NO: 5547)
PLNGAQMLY,
(SEQ ID NO: 5548)
PLNGVKYLY,
(SEQ ID NO: 5597)
PLNMPGHLY,
(SEQ ID NO: 5551)
PLNGVQALY,
(SEQ ID NO: 4792)
SLNGAVHLY,
(SEQ ID NO: 5552)
PLNGVKTLY,
(SEQ ID NO: 5598)
SINGPVHLY,
(SEQ ID NO: 5599)
PLNLVGHLY,
(SEQ ID NO: 5600)
PLNLTGHLY,
or
(SEQ ID NO: 5601)
PGRQAVHLY;

(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
303. The AAV capsid variant of any one of embodiments 292 or 297-302, wherein [A], [B], and/or [C] is present in loop VIII of the AAV capsid variant.
304. The AAV capsid variant of any one of embodiments 292 or 297-303, wherein [A] is present immediately subsequent to position 586, numbered according to SEQ ID NO: 5, 8, 138, or 3636.
305. The AAV capsid variant of any one of embodiments 292 or 297-304, wherein [A] replaces positions 587 and 588 (e.g., A 587 and Q588), numbered according to SEQ ID NO: 138.
306. The AAV capsid variant of any one of embodiments 292 or 297-305, wherein [A] is present immediately subsequent to position 586 and replaces positions 587 and 588 (e.g., A 587 and Q588), numbered according to SEQ ID NO: 138.
307. The AAV capsid variant of any one of embodiments 292 or 297-129, wherein [A] corresponds to positions 587-590 of SEQ ID NO: 5, 8, or 3636.
308. The AAV capsid variant of any one of embodiments 292 or 297-307, wherein [B] is present immediately subsequent to [A].
309. The AAV capsid variant of any one of embodiments 291-308, wherein [B] corresponds to positions 591-593 of SEQ ID NO: 5, 8, or 3636.
310. AAV capsid variant of any one of embodiments 292 or 297-309, [A]-[B] is present immediately subsequent to position 586, numbered according to SEQ ID NO: 138.
311. The AAV capsid variant of any one of embodiments 292 or 297-310, wherein [A]-[B] replaces positions 587 and 588 (e.g., A 587 and Q588), numbered according to SEQ ID NO: 138.
312. The AAV capsid variant of any one of embodiments 292 or 297-311, wherein [A]-[B] is present immediately subsequent to position 586 and replaces positions 587 and 588 (e.g., A 587 and Q588), numbered according to SEQ ID NO: 138.
313. The AAV capsid variant of any one of embodiments 292 or 297-312, wherein [A]-[B] corresponds to positions 587-593 of SEQ ID NO: 5, 8, or 3636.
314. The AAV capsid variants of any one of embodiments 291-313, wherein [C] is present immediately subsequent to [B].
315. The AAV capsid variants of any one of embodiments 291-314, wherein [C] corresponds to positions 594 and 595 of SEQ ID NO: 5, 8, or 3636.
316. The AAV capsid variants of any one of embodiments 292 or 297-315, wherein [B]-[C] is present immediately subsequent to [A].
317. The AAV capsid variants of any one of embodiments 292 or 297-318, wherein [A]-[B]-[C] is present immediately subsequent to position 586, numbered according to SEQ ID NO: 138.
318. The AAV capsid variants of any one of embodiments 292 or 297-319, wherein [A]-[B]-[C] replaces positions 587 and 588 (e.g., A 587 and Q588), numbered according to SEQ ID NO: 138.
319. The AAV capsid variants of any one of embodiments 292 or 297-320, wherein [A]-[B]-[C] is present immediately subsequent to position 586 and replaces positions 587 and 588 (e.g., A 587 and Q588), numbered according to SEQ ID NO: 138.
320. The AAV capsid variants of any one of embodiments 292 or 297-321, wherein [A]-[B]-[C] corresponds to positions 587-595 of SEQ ID NO: 5, 8, or 3636.
321. An AAV capsid variant comprising an amino acid sequence comprising positions X1-X2-X3-X4-X5-X6-X7-X8-X9, wherein:
   (i) X1 is: P, T, S, A, or L;
   (ii) X2 is: L, S, I, G, or V;
   (iii) X3 is: N, A, or R;
   (iv) X4 is: N, G, R, M, L, Q, or S;
   (v) X5 is: P, V, A, T, or G;
   (vi) X6 is: G, K, R, Q, or V;
   (vii) X7 is: H, A, M, S, T, Q, Y, or R;
   (viii) X8 is: L, I, D, or V; and
   (ix) X9 is: Y, N, or S.
322. The AAV capsid variant of embodiment 321, wherein X1-X2-X3-X4-X5-X6-X7-X8-X9 is present immediately subsequent to position 586, numbered according to SEQ ID NO: 5, 8, 138, or 3636.
323. The AAV capsid variant of embodiment 321 or 322, wherein X1-X2-X3-X4-X5-X6-X7-X8-X9 replaces positions 587 and 588 (e.g., A 587 and Q588), numbered according to SEQ ID NO: 138.
324. The AAV capsid variant of any one of embodiments 321-232, wherein X1-X2-X3-X4-X5-X6-X7-X8-X9 is present immediately subsequent to position 586 and replaces positions 587 and 588 (e.g., A 587 and Q588), numbered according to SEQ ID NO: 138.
325. The AAV capsid variant of any one of embodiments 321-324, wherein X1-X2-X3-X4-X5-X6-X7-X8-X9 corresponds to positions 587-595 of SEQ ID NO: 5, 8, or 3636.
326. The AAV capsid variant of any one of embodiments 259-290, which further comprises an amino acid other than A at position 589 (e.g., V) or an amino acid other than G at position 594 (e.g., R), relative to a reference sequence numbered according to the amino acid sequence of SEQ ID NO: 138.
327. The AAV capsid variant of any one of embodiments 291-320, which further comprises an amino acid other than A at position 589 (e.g., V) or an amino acid other than W at position 595 (e.g., S), relative to a reference sequence numbered according to the amino acid sequence of SEQ ID NO: 138.
328. The AAV capsid variant of any one of embodiments 321-325, which further comprises an amino acid other than A at position 589 (e.g., V), an amino acid other than A at position 591 (e.g., G or V), an amino acid other than T at position 593 (e.g., A), an amino acid other than G at position 594 (e.g., R), or an amino acid other than W at position 595 (e.g., S), relative to a reference sequence numbered according to the amino acid sequence of SEQ ID NO: 138.
329. The AAV capsid variant of any one of embodiments 259-328, which further comprises the amino acid V at position 589, relative to a reference sequence numbered according to the amino acid sequence of SEQ ID NO: 138.
330. The AAV capsid variant of any one of embodiments 321, 328, or 329, which further comprises:
   (i) the amino acid G at position 591, relative to a reference sequence numbered according to the amino acid sequence of SEQ ID NO: 138; or
   (ii) the amino acid V at position 591, relative to a reference sequence numbered according to the amino acid sequence of SEQ ID NO: 138.
331. The AAV capsid variant of any one of embodiments 321, or 328-330, which further comprises the amino acid A position 593, relative to a reference sequence numbered according to the amino acid sequence of SEQ ID NO: 138.
332. The AAV capsid variant of any one of embodiments 259-290, 321-326, or 328-331, which further comprises the amino acid R at position 594, relative to a reference sequence numbered according to the amino acid sequence of SEQ ID NO: 138.
333. The AAV capsid variant of any one of embodiments 291-326, 327-329, which further comprises the amino acid S at position 595, relative to a reference sequence numbered according to the amino acid sequence of SEQ ID NO: 138.
334. The AAV capsid variant of any one of embodiments 259-290, 326, 329, or 332, wherein the amino acid sequence comprises:
   (i) the amino acid sequence of any of SEQ ID NOs: 1140-1151, 1153-1157, 1159, 1161, 1163, 1166, 1167;
   (ii) an amino acid sequence comprising any portion of an amino acid sequence in (i), e.g., any 2, 3, 4, 5, 6, 7, 8, 9, 10, 11, 12, 13, 14, or 15 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
335. The AAV capsid variant of any one of embodiments 291-325, 327, 329, or 333, wherein the amino acid sequence comprises:
   (i) the amino acid sequence of any of SEQ ID NOs: 1139, 1140, 1142-1155, 1156, 1160, 1161-1163, 1165, or 1169-1171;
   (ii) an amino acid sequence comprising any portion of an amino acid sequence in (i), e.g., any 2, 3, 4, 5, 6, 7, 8, 9, 10, 11, 12, 13, 14, or 15 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
336. The AAV capsid variant of embodiment 321, or 328-333, wherein the amino acid sequence comprises:
   (i) the amino acid sequence of any of SEQ ID NOs: 1139-1172;
   (ii) an amino acid sequence comprising any portion of an amino acid sequence in (i), e.g., any 2, 3, 4, 5, 6, 7, 8, 9, 10, 11, 12, 13, 14, or 15 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
337. An AAV capsid variant comprising:
   (a) the amino acid sequence of any one of the sequences provided in Tables 1A, 1B, 10, or 20;
   (b) an amino acid sequence comprising at least 3, 4, 5, 6, 7, 8, 9, 10, 11, 12, 13, 14, 15, 16, 17, or 18 consecutive amino acids from any one of the sequences provided in Tables 1A, 1B, 10, or 20; or
   (c) an amino acid sequence comprising at least one, two, or three but no more than four different amino acids, relative to any one of the amino acid sequences provided in Tables 1A, 1B, 10, or 20; or
   (d) an amino acid sequence comprising at least one, two, or three but no more than four modifications, e.g., substitutions (e.g., conservative substitutions), insertions, or deletions, relative to the amino acid sequence of any one of the sequences provided in Tables 1A, 1B, 10, or 20;
   optionally wherein the AAV capsid variant does not comprise the amino acid sequence of TGW at positions 593-595, relative to a reference sequence numbered according to the amino acid sequence of SEQ ID NO: 138.
338. An AAV capsid variant comprising:
   (a) the amino acid sequence of any one of SEQ ID NOs: 139-1138;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; or
   (c) an amino acid sequence comprising at least one, two, or three but no more than four different amino acids, relative to the amino acid sequence of any one of SEQ ID NOs: 139-1138;
   (d) an amino sequence comprising at least one, two, or three but no more than four modifications, e.g., substitutions (e.g., conservative substitutions), insertions, or deletions, relative to the amino acid sequence of any one of SEQ ID NOs: 139-1138;
   optionally wherein the AAV capsid variant does not comprise the amino acid sequence of TGW at positions 593-595, relative to a reference sequence numbered according to the amino acid sequence of SEQ ID NO: 138.
339. An AAV capsid variant comprising:
   (a) the amino acid sequence of any one of SEQ ID NOs: 139-476;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or
   (c) an amino acid sequence comprising at least one, two, or three but no more than four different amino acids, relative to the amino acid sequence of any one of SEQ ID NOs: 139-476;
   (d) an amino sequence comprising at least one, two, or three but no more than four modifications, e.g., substitutions (e.g., conservative substitutions), insertions, or deletions, relative to the amino acid sequence of any one of SEQ ID NOs: 139-476;
   optionally wherein the AAV capsid variant does not comprise the amino acid sequence of TGW at positions 593-595, relative to a reference sequence numbered according to the amino acid sequence of SEQ ID NO: 138.
340. An AAV capsid variant comprising:
   (a) the amino acid sequence of any one of the amino acid sequences provided in Table 1B;
   (b) an amino acid sequence comprising at least 3, 4, 5, 6, 7, 8, 9, 10, 11, 12, 13, 14, 15, 16, 17, or 18 consecutive amino acids from any one of the amino acid sequences provided in Table 1B; or
   (c) an amino acid sequence comprising at least one, two, or three but no more than four different amino acids, relative to the amino acid sequence of any one of the amino acid sequences provided in Table 1B;
   (d) an amino sequence comprising at least one, two, or three but no more than four modifications, e.g., substitutions (e.g., conservative substitutions), insertions, or deletions, relative to the amino acid sequence of any one of the amino acid sequences provided in Table 1B;
   optionally wherein the AAV capsid variant does not comprise the amino acid sequence of TGW at positions 593-595, relative to a reference sequence numbered according to the amino acid sequence of SEQ ID NO: 138.
341. The AAV capsid variant of embodiment 337 or 338, comprising an amino acid sequence comprising at least 4, 5, 6, 7, 8, 9, 10, 11, 12, 13, 14, 15, 16, 17, or 18 consecutive amino acids from any one of SEQ ID NOs: 139-1138.
342. The AAV capsid variant of any one of embodiments 337-341, comprising an amino acid sequence comprising at least 4, 5, 6, 7, 8, 9, 10, 11, 12, 13, 14, 15, 16, 17, or 18 consecutive amino acids from SEQ ID NO: 314.
343. The AAV capsid variant of any one of embodiments 337-341, comprising an amino acid sequence comprising at least 4, 5, 6, 7, 8, 9, 10, 11, 12, 13, 14, 15, 16, 17, or 18 consecutive amino acids from SEQ ID NO: 566.
344. The AAV capsid variant of any one of embodiments 337-339, or 340, comprising an amino acid sequence comprising at least 4, 5, 6, 7, 8, 9, 10, 11, 12, 13, 14, 15, 16, 17, or 18 consecutive amino acids from any one of SEQ ID NOs: 139-476.
345. The AAV capsid variant of embodiment 337 or 338, comprising an amino acid sequence comprising at least 4, 5, 6, 7, 8, 9, 10, 11, 12, 13, 14, 15, 16, 17, or 18 consecutive amino acids from any one of the sequences provided in Table 1B.
346. The AAV capsid variant of embodiment 337 or 338, which comprises an amino acid sequence comprising at least one, two, or three but no more than four different amino acids relative to the amino acid sequence of any one of SEQ ID NOs: 139-1138.
347. The AAV capsid variant of any one of embodiments 337-339, which comprises an amino acid sequence comprising at least one, two, or three but no more than four different amino acids relative to the amino acid sequence of any one of SEQ ID NOs: 139-476.
348. The AAV capsid variant of embodiment 337 or 338, which comprises an amino acid sequence comprising at least one, two, or three but no more than four different amino acids relative to the amino acid sequence of any one of the amino acid sequences provided in Table 1B.
349. The AAV capsid variant of any one of embodiments 337, 338, or 346, which comprises an amino acid sequence comprising at least one, two, or three but no more than four modifications, e.g., substitutions (e.g., conservative substitutions), insertions, or deletions, relative to the amino acid sequence of SEQ ID NOs: 139-1138.
350. The AAV capsid variant of any one of embodiments 337, 338, 346, or 349, which comprises:
   (i) an amino acid sequence comprising at least one, two, or three but no more than four different amino acids relative to the amino acid sequence of SEQ ID NO: 314; or
   (ii) an amino acid sequence comprising at least one, two, or three but no more than four modifications, e.g., substitutions (e.g., conservative substitutions), insertions, or deletions, relative to SEQ ID NO: 314.
351. The AAV capsid variant of any one of embodiments 337, 338, 346, or 349, which comprises:
   (i) an amino acid sequence comprising at least one, two, or three but no more than four different amino acids relative to the amino acid sequence of SEQ ID NO: 566; or
   (ii) an amino acid sequence comprising at least one, two, or three but no more than four modifications, e.g., substitutions (e.g., conservative substitutions), insertions, or deletions, relative to SEQ ID NO: 566.
352. The AAV capsid variant of any one of embodiments 337-339 or 347, which comprises an amino acid sequence comprising at least one, two, or three but no more than four modifications, e.g., substitutions (e.g., conservative substitutions), insertions, or deletions, relative to the amino acid sequence of SEQ ID NOs: 139-476.
353. The AAV capsid variant of any one of embodiments 337, 340, or 348, which comprises an amino acid sequence comprising at least one, two, or three but no more than four modifications, e.g., substitutions (e.g., conservative substitutions), insertions, or deletions, relative to any of the amino acid sequences provided in Table 1B.
354. The AAV capsid variant of any one of embodiments 337-353, wherein 4, 5, 6, 7, 8, or 9 consecutive amino acids is not PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).
355. The AAV capsid variant of embodiment 337, 338, 341-343, 346, 349, or 354, which comprises the amino acid sequence of any one of SEQ ID NOs: 139-1138.
356. The AAV capsid variant of embodiment 337, 338, 341, 342, 346, 349, or 354, which comprises the amino acid sequence of SEQ ID NO: 314.
357. The AAV capsid variant of embodiment 337, 338, 341, 343, 346, 349, or 354, which comprises the amino acid sequence of SEQ ID NO: 566.
358. The AAV capsid variant of embodiment 337-339, 344, 347, 352, or 354, which comprises the amino acid sequence of any one of SEQ ID NOs: 139-476.
359. The AAV capsid variant of embodiment 337, 340, 345, 348, 353, or 354, which comprises the amino acid sequence of any one of the amino acid sequences provided in Table 1B.
360. An AAV capsid variant comprising:
   (a) the amino acid sequence of any one of SEQ ID NOs: 1139-1172;
   (b) an amino acid sequence comprising at least 3, 4, 5, 6, 7, 8, 9, 10, 11, 12, 13, 14, or 15 consecutive amino acids from any one of SEQ ID NOs: 1139-1172; or
   (c) an amino acid sequence comprising at least one, two, or three but no more than four different amino acids, relative to the amino acid sequence of any one of SEQ ID NOs: 1139-1172;
   (d) an amino sequence comprising at least one, two, or three but no more than four modifications, e.g., substitutions (e.g., conservative substitutions), insertions, or deletions, relative to the amino acid sequence of any one of SEQ ID NOs: 1139-1172.
361. The AAV capsid variant of any one of embodiments 337-360, wherein the amino acid sequence is present in loop VIII.
362. The AAV capsid variant of any one of embodiments 337-361, wherein the amino acid sequence is present immediately subsequent to position 586, 587, 588, 589, 590, 591, 592, 593, 594, or 595, relative to a reference sequence numbered according to the amino acid sequence of SEQ ID NO: 138.
363. The AAV capsid variant of any one of embodiments 337-362, wherein the amino acid sequence is present immediately subsequent to position 586, relative to a reference sequence numbered according to the amino acid sequence of SEQ ID NO: 138.
364. The AAV capsid variant of any one of embodiments 337-363, wherein the amino acid sequence replaces positions 587 and 588 (e.g., A587 and Q588), numbered according to SEQ ID NO: 138.
365. The AAV capsid variant of any one of embodiments 337-362, wherein the amino acid sequence is present immediately subsequent to position 588, relative to a reference sequence numbered according to the amino acid sequence of SEQ ID NO: 138.
366. The AAV capsid variant of any one of embodiments 337-362, wherein the amino acid sequence is present immediately subsequent to position 592, relative to a reference sequence numbered according to the amino acid sequence of SEQ ID NO: 138.
367. The AAV capsid variant of any one of embodiments 337-362, wherein the amino acid sequence is present immediately subsequent to position 595, relative to a reference sequence numbered according to the amino acid sequence of SEQ ID NO: 138.
368. The AAV capsid variant of any one of embodiments 1-367, which further comprises an amino acid other than A at position 587 and an amino acid other than Q at position 588, relative to a reference sequence numbered according to the amino acid sequence of SEQ ID NO: 138.
369. The AAV capsid variant of any one of embodiments 337-368, which further comprises:
   (i) one, two, three, or all of an amino acid other than A at position 589, an amino acid other than Q at position 590, an amino acid other than A at position 591, and/or an amino acid other than Q at position 592, relative to a reference sequence numbered according to the amino acid sequence of SEQ ID NO: 138;
   (ii) one, two, or all of an amino acid other than T at position 593, an amino acid other than G at position 594, and/or an amino acid other than W at position 595, relative to a reference sequence numbered according to the amino acid sequence of SEQ ID NO: 138; and/or
   (iii) one, two, or all of an amino acid other than V at position 596, an amino acid other than Q at position 597, and/or an amino acid other than N at position 598, relative to a reference sequence numbered according to the amino acid sequence of SEQ ID NO: 138.
370. The AAV capsid variant of any one of embodiments 337-369, which comprises the amino acid P at position 587 the amino acid L at position 588, and the amino acid sequence NGAVHLY (SEQ ID NO: 3689), which is present immediately subsequent to position 588, corresponding to or numbered according to SEQ ID NO: 5, 8, 138, or 3636.
371. An AAV capsid variant comprising the amino acid P at position 587 the amino acid L at position 588, and the amino acid sequence NGAVHLY (SEQ ID NO: 3689) present immediately subsequent to position 588, corresponding to or numbered according to SEQ ID NO: 5, 8, 138, or 3636.
372. The AAV capsid variant of any one of embodiments 337-371, which further comprises:
   (i) one, two, or all of an amino acid other than T at position 593, an amino acid other than G at position 594, and/or an amino acid other than W at position 595, numbered according to the amino acid sequence of SEQ ID NO: 138; or
   (ii) one, two, or all of an amino acid other than T at position 600, an amino acid other than G at position 601, and/or an amino acid other than W at position 602, numbered according to the amino acid sequence of SEQ ID NO: 5, 8, or 3636.
373. The AAV capsid variant of any one of embodiments 337-372, which further comprises:
   (i) an amino acid other than T at position 593, an amino acid other than G at position 594, and an amino acid other than W at position 595, numbered according to the amino acid sequence of SEQ ID NO: 138;
   (ii) an amino acid other than T at position 600, an amino acid other than G at position 601, and an amino acid other than W at position 602, numbered according to the amino acid sequence of SEQ ID NO: 5, 8, 3636.
374. The AAV capsid variant of any one of embodiments 337-373, which further comprises:
   (i) one, two, or all of the amino acid L at position 593, the amino acid S at position 594, and/or the amino acid P at position 595, numbered according to the amino acid sequence of SEQ ID NO: 138; or
   (ii) one, two, or all of the amino acid L at position 600, the amino acid S at position 601, and/or the amino acid P at position 602, numbered according to the amino acid sequence of SEQ ID NO: 5, 8, or 3636.
375. The AAV capsid variant of any one of embodiments 337-374, which further comprises:
   (i) the amino acid L at position 593, the amino acid S at position 594, and the amino acid P at position 595, numbered according to the amino acid sequence of SEQ ID NO: 138; or
   (ii) the amino acid L at position 600, the amino acid S at position 601, and the amino acid P at position 602, numbered according to the amino acid sequence of SEQ ID NO: 5, 8, or 3636.
376. The AAV capsid variant of any one of embodiments 337-375, which further comprises:
   (i) one, two, or all of an amino acid other than V at position 596, an amino acid other than Q at position 597, and/or an amino acid other than N at position 598, numbered according to the amino acid sequence of SEQ ID NO: 138; or
   (ii) one, two, or all of an amino acid other than V at position 603, an amino acid other than Q at position 604, and/or an amino acid other than N at position 605, numbered according to the amino acid sequence of SEQ ID NO: 5, 8, 3636.
377. The AAV capsid variant of any one of embodiments 337-376, which further comprises an amino acid other than Q at position 597 numbered according to the amino acid sequence of SEQ ID NO: 138 or at position 604 numbered according to SEQ ID NO: 5, 8, or 3636.
378. The AAV capsid variant of any one of embodiments 337-376, which further comprises the amino acid P at position 597 numbered according to the amino acid sequence of SEQ ID NO: 138 or at position 604 numbered according to SEQ ID NO: 5, 8, or 3636.
379. The AAV capsid variant of any one of embodiments 337-376, which further comprises the amino acid K at position 597 numbered according to the amino acid sequence of SEQ ID NO: 138 or at position 604 numbered according to SEQ ID NO: 5, 8, or 3636.
380. The AAV capsid variant of any one of embodiments 337-376, which further comprises the amino acid E or H at position 597 numbered according to the amino acid sequence of SEQ ID NO: 138 or at position 604 numbered according to SEQ ID NO: 5, 8, or 3636.
381. The AAV capsid variant of any one of embodiments 337-380, which further comprises the amino acid L at position 593, the amino acid S at position 594, the amino acid P at position 595, and the amino acid K at position 597, numbered according to the amino acid sequence of SEQ ID NO: 138.
382. The AAV capsid variant of any one of embodiments 337-377, 379, or 381, which further comprises the amino acid L at position 600, the amino acid S at position 601, the amino acid P at position 602, and the amino acid K at position 604, corresponding to or numbered according to the amino acid sequence of SEQ ID NO: 8 or 3636.
383. The AAV capsid variant of any one of embodiments 337-377, 379, 381, or 382, which comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
384. The AAV capsid variant of any one of embodiments 337-377, 379, or 381-383, which comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
385. The AAV capsid variant of any one of embodiments 337-377, 379, or 381-384, which comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 8 or 3636; and
   (ii) the amino acid L at position 600, the amino acid S at position 601, the amino acid P at position 602, and the amino acid K at position 604, corresponding to or numbered according to the amino acid sequence of SEQ ID NO: 8 or 3636.
386. The AAV capsid variant of any one of embodiments 337-378, which further comprises the amino acid P at position 597, numbered according to the amino acid sequence of SEQ ID NO: 138.
387. The AAV capsid variant of any one of embodiments 337-378 or 386, which further comprises the amino acid P at position 604, corresponding to or numbered according to the amino acid sequence of SEQ ID NO: 5 or 3636.
388. The AAV capsid variant of any one of embodiments 337-378, 386, or 387, which comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid P at position 597, numbered according to the amino acid sequence of SEQ ID NO: 138.
389. The AAV capsid variant of any one of embodiments 337-378 or 386-388, which comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid P at position 597, numbered according to the amino acid sequence of SEQ ID NO: 138.
390. The AAV capsid variant of any one of embodiments 337-378 or 386-389, which comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 5 or 3636; and
   (ii) the amino acid P at position 604, corresponding to or numbered according to the amino acid sequence of SEQ ID NO: 5 or 3636.
391. An AAV capsid variant comprising:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
392. An AAV capsid variant comprising:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
393. An AAV capsid variant comprising:
   (i) the amino acid P at position 587, the amino acid L at position 588, and the amino acid sequence NGAVHLY (SEQ ID NO: 3689), which is present immediately subsequent to position 588, numbered according to SEQ ID NO: 8 or 3636; and
   (ii) the amino acid L at position 600, the amino acid S at position 601, the amino acid P at position 602, and the amino acid K at position 604, corresponding to or numbered according to the amino acid sequence of SEQ ID NO: 8 or 3636.
394. An AAV capsid variant comprising:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid P at position 597, numbered according to the amino acid sequence of SEQ ID NO: 138.
395. An AAV capsid variant comprising:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid P at position 597, numbered according to the amino acid sequence of SEQ ID NO: 138.
396. An AAV capsid variant comprising:
   (i) the amino acid P at position 587, the amino acid L at position 588, and the amino acid sequence NGAVHLY (SEQ ID NO: 3689), which is present immediately subsequent to position 588, numbered according to SEQ ID NO: 5 or 3636; and
   (ii) the amino acid P at position 604, corresponding to or numbered according to the amino acid sequence of SEQ ID NO: 5 or 3636.
397. The AAV capsid variant, of any one of the preceding embodiments, which further comprises:
   (i) a modification, e.g., an insertion, substitution (e.g., conservative substitution), and/or deletion, in loop I, II, IV, and/or VI; and/or
   (ii) a substitution at position K449, e.g., a K449R substitution, numbered according to SEQ ID NO: 138.
398. The AAV capsid variant of any one of the preceding embodiments, which comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 138.
399. The AAV capsid variant, of any one of the preceding embodiments, which comprises an amino acid sequence comprising at least one, two or three, but no more than 30, 20 or 10 different amino acids relative to the amino acid sequence of SEQ ID NO: 138.
400. The AAV capsid variant, of any one of the preceding embodiments, which comprises the amino acid sequence of SEQ ID NO: 138, or an amino acid sequence with at least 80% (e.g., at least about 85, 90, 95, 96, 97, 98, or 99%) sequence identity thereto.
401. The AAV capsid variant, of any one of the preceding embodiments, which comprises the amino acid sequence of SEQ ID NO: 138.
402. The AAV capsid variant, of any one of the preceding embodiments, which comprises an amino acid sequence encoded by the nucleotide sequence of SEQ ID NO: 137, or a sequence with at least 80% (e.g., at least about 85, 90, 95, 96, 97, 98, or 99%) sequence identity thereto.
403. The AAV capsid variant, of any one of the preceding embodiments, wherein the nucleotide sequence encoding the capsid variant comprises the nucleotide sequence of SEQ ID NO: 137, or a sequence with at least 80% (e.g., at least about 85, 90, 95, 96, 97, 98, or 99%) sequence identity thereto.
404. The AAV capsid variant, of any one of the preceding embodiments, which comprises the amino acid sequence of SEQ ID NO: 5 or 8, or a sequence with at least 80% (e.g., at least about 85, 90, 95, 96, 97, 98, or 99%) sequence identity thereto.
405. The AAV capsid variant, of any one of the preceding embodiments, wherein the nucleotide sequence encoding the capsid variant comprises the nucleotide sequence of SEQ ID NO: 4 or 7, or a sequence with at least 80% (e.g., at least about 85, 90, 95, 96, 97, 98, or 99%) sequence identity thereto.
406. The AAV capsid variant, of any one of the preceding embodiments, which comprises a VP1 protein, a VP2 protein, a VP3 protein, or a combination thereof, optionally wherein the AAV capsid variant comprises:
   (i) the amino acid sequence corresponding to positions 138-743, e.g., a VP2, of SEQ ID NO: 5, 8, or 3636, or a sequence with at least 80% (e.g., at least about 85, 90, 95, 96, 97, 98, or 99%) sequence identity thereto;
   (ii) the amino acid sequence corresponding to positions 138-736, e.g., a VP2, of SEQ ID NO: 138, or a sequence with at least 80% (e.g., at least about 85, 90, 95, 96, 97, 98, or 99%) sequence identity thereto;
   (iii) the amino acid sequence corresponding to positions 203-743, e.g., a VP3, of SEQ ID NO: 5, 8, or 3636, or a sequence with at least 80% (e.g., at least about 85, 90, 95, 96, 97, 98, or 99%) sequence identity thereto; and/or
   (iv) the amino acid sequence corresponding to positions 203-736, e.g., a VP3, of SEQ ID NO: 138, or a sequence with at least 80% (e.g., at least about 85, 90, 95, 96, 97, 98, or 99%) sequence identity thereto.
407. An AAV capsid variant comprising the amino acid sequence of SEQ ID NO: 5.
408. The AAV capsid variant of embodiment 407, wherein the nucleotide sequence encoding the AAV capsid variant comprises the nucleotide sequence of SEQ ID NO: 4.
409. An AAV capsid variant comprising the amino acid sequence of SEQ ID NO: 8.
410. The AAV capsid variant of embodiment 409, wherein the nucleotide sequence encoding the AAV capsid variant comprises the nucleotide sequence of SEQ ID NO: 7.
411. The AAV capsid variant, of any one of the preceding embodiments, which does not comprise:
   (i) the amino acid sequence of TLAVPFK (SEQ ID NO: 1262) present immediately subsequent to position 588, numbered according to SEQ ID NO: 138;
   (ii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any the amino acid sequences provided in Table 1 of WO2020223276, the contents of which are hereby incorporated by reference in their entirety; or
   (iii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any SEQ ID NOs: 1, 3, 12, 13, or 138.
412. The AAV capsid variant, of any one of the preceding embodiments, which has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.
413. The AAV capsid variant, of any one of the preceding embodiments, which has an increased tropism for a CNS cell or tissue, e.g., a brain cell, brain tissue, spinal cord cell, or spinal cord tissue, relative to the tropism of a reference sequence comprising the amino acid sequence of SEQ ID NO: 3636.
414. The AAV capsid variant of any one of embodiments 1-413, which is enriched at least about 1.1, 1.2, 1.3, 1.4, 1.5, 1.6, 1.7, 1.8, 1.9, 2, 2.5, 3, 3.5, 4, 4.5, 5, 5.5, or 6-fold, in the brain compared to a reference sequence of SEQ ID NO: 3636, e.g., when measured by an assay as described in Example 7.
415. The AAV capsid variant of any one of embodiments 1-413, which is enriched at least about 2, 2.5, 3, 3.5, 4, 4.5, 5, 5.5, or 6-fold, in the brain compared to a reference sequence of SEQ ID NO: 3636, e.g., when measured by an assay as described in Example 7.
416. The AAV capsid variant of any one of embodiments 62-118, 337, 340, 345, 348, 353, 354, or 359, which results in greater than 1, 2, 5, 10, 20, 30, 40, 50, or 100 reads per sample, e.g., when analyzed by an NGS sequencing assay, e.g., as described in Example 7.
417. The AAV capsid variant of any one of embodiments 1-416, which is enriched in the brain of at least two to three species, e.g., a non-human primate and rodent (e.g., mouse), e.g., as compared to a reference sequence of SEQ ID NO: 138.
418. The AAV capsid variant of embodiment 417, wherein the at least two to three species are Macaca fascicularis, Chlorocebus sabaeus, Callithrix jacchus, and/or mouse (e.g., BALB/c mice).
419. The AAV capsid variant of any one of the preceding embodiments, which further comprises a modification, e.g., substitution (e.g., conservative substitution), insertion, or deletion, that results in one, two, three or all of: (1) reduced tropism in the liver; (2) de-targeted expression in the liver; (3) reduced activity in the liver; and/or (4) reduced binding to galactose.
420. The AAV capsid variant of any one of the preceding embodiments, which further comprises:
   (i) a modification e.g., substitution (e.g., conservative substitution), insertion, or deletion, at position N470 (e.g., N470A), D271 (e.g., D271A), N272 (e.g., N297A), Y446 (e.g., Y446A), N498 (e.g., N498Y or N498I), W503 (e.g., W530R or W530A), L620 (e.g., L620F), or a combination thereof, relative to a reference sequence numbered according to SEQ ID NO: 138; or
   (ii) one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498 (e.g., Y or I), and amino acid other than W at position 503 (e.g., R or A), and amino acid other than L at position 620 (e.g., F), relative to a reference sequence numbered according to SEQ ID NO: 138.
421. A polynucleotide encoding the AAV capsid variant of any one of embodiments 1-420.
422. The polynucleotide of embodiment 421, which comprises a nucleotide sequence that is codon optimized.
423. A peptide comprising:
   (a) the amino acid sequence of any of the sequences provided in Tables 1A, 1B, 10, or 20;
   (b) an amino acid sequence comprising at least 3, 4, 5, 6, 7, 8, 9, 10, 11, 12, 13, 14, 15, 16, 17, 18, 19, 20 consecutive amino acids from any one of the sequences provided in Tables 1A, 1B, 10, or 20;
   (c) an amino acid sequence comprising at least one, two, or three but no more than four different amino acids relative to the amino acid sequence of any one of the sequences provided in Tables 1A, 1B, 10, or 20; or
   (d) an amino acid sequence comprising at least one, two, or three but no more than four modifications, e.g., substitutions (e.g., conservative substitutions), relative to the amino acid sequence of any one of the sequences provided in Tables 1A, 1B, 10, or 20;
   optionally provided that the amino acid sequence is not PLN, PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).
424. A peptide comprising:
   (a) the amino acid sequence of any one of SEQ ID NOs: 139-476;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or
   (c) an amino acid sequence comprising at least one, two, or three but no more than four different amino acids, relative to the amino acid sequence of any one of SEQ ID NOs: 139-476;
   (d) an amino sequence comprising at least one, two, or three but no more than four modifications, e.g., substitutions (e.g., conservative substitutions), relative to the amino acid sequence of any one of SEQ ID NOs: 139-476;
   optionally provided that the amino acid sequence is not PLN, PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).
425. A peptide comprising one, two, three, four, or all of:
   (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722);
   (ii) an [N2] wherein [N2] is or comprises: VHLY (SEQ ID NO: 4680), VHVY (SEQ ID NO: 4682), VPLY (SEQ ID NO: 4723), VNLY (SEQ ID NO: 4724), VHRY (SEQ ID NO: 4725), VHIY (SEQ ID NO: 4681), VHHY (SEQ ID NO: 4683), FHLY (SEQ ID NO: 4726), LHLY (SEQ ID NO: 4727), DHLY (SEQ ID NO: 4728), VQLY (SEQ ID NO: 4729), IHLY (SEQ ID NO: 4730), VDLY (SEQ ID NO: 4731), AHLY (SEQ ID NO: 4732), VLLY (SEQ ID NO: 4733), GHLY (SEQ ID NO: 4734), VRLY (SEQ ID NO: 4735), or VYLY (SEQ ID NO: 4736);
   (iii) an [N3] wherein [N3] is or comprises: AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or wherein the AAV capsid variant comprises an amino acid modification, e.g., a conservative substitution, of any of the aforesaid amino acids in (i)-(v).
426. A polynucleotide encoding an AAV capsid variant comprising:
   (a) the amino acid sequence of any one of SEQ ID NOs: 139-476;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or
   (c) an amino acid sequence comprising at least one, two, or three but no more than four different amino acids, relative to the amino acid sequence of any one of SEQ ID NOs: 139-476;
   (d) an amino sequence comprising at least one, two, or three but no more than four modifications, e.g., substitutions (e.g., conservative substitutions), relative to the amino acid sequence of any one of SEQ ID NOs: 139-476;
   optionally wherein:
   (i) the amino acid sequence of (a), (b), (c), and/or (d) is present immediately subsequent to position 586, 587, 588, 589, 590, 591, 592, 593, 594, or 595, relative to a reference sequence numbered according to the amino acid sequence of SEQ ID NO: 138; or
   (ii) the amino acid sequence is not PLN, PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).
427. A polynucleotide encoding an AAV capsid variant comprising:
   (i) an [N1], wherein [N1] is or comprises: PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722);
   (ii) an [N2] wherein [N2] is or comprises: VHLY (SEQ ID NO: 4680), VHVY (SEQ ID NO: 4682), VPLY (SEQ ID NO: 4723), VNLY (SEQ ID NO: 4724), VHRY (SEQ ID NO: 4725), VHIY (SEQ ID NO: 4681), VHHY (SEQ ID NO: 4683), FHLY (SEQ ID NO: 4726), LHLY (SEQ ID NO: 4727), DHLY (SEQ ID NO: 4728), VQLY (SEQ ID NO: 4729), IHLY (SEQ ID NO: 4730), VDLY (SEQ ID NO: 4731), AHLY (SEQ ID NO: 4732), VLLY (SEQ ID NO: 4733), GHLY (SEQ ID NO: 4734), VRLY (SEQ ID NO: 4735), or VYLY (SEQ ID NO: 4736);
   (iii) an [N3] wherein [N3] is or comprises: AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or
   (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or
   wherein the AAV capsid variant comprises an amino acid modification, e.g., a conservative substitution, of any of the aforesaid amino acids in (i)-(v).
428. The polynucleotide, peptide, or AAV capsid variant, of any one of embodiments 1-427, which is isolated, e.g., recombinant.
429. An AAV particle comprising the AAV capsid variant of any one of embodiments 1-420 or an AAV capsid variant comprising the peptide of any one of embodiments 423-425.
430. The AAV particle of embodiment 429, which comprises a nucleotide sequence encoding a payload.
431. The AAV particle of embodiment 460, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
432. The AAV particle of embodiment 431, wherein the therapeutic protein or functional variant thereof, e.g., a recombinant protein, is associated with (e.g., aberrantly expressed in) a neurological or neurodegenerative disorder, a muscular or neuromuscular disorder, or a neuro-oncological disorder.
433. The AAV particle of embodiment 430 or 431, the therapeutic protein or functional variant thereof is chosen from apolipoprotein E (APOE) (e.g., ApoE2, ApoE3 and/or ApoE4); human survival of motor neuron (SMN) 1 or SMN2; glucocerebrosidase (GBA1); aromatic L-amino acid decarboxylase (AADC); aspartoacylase (ASPA); tripeptidyl peptidase I (CLN2); beta-galactosidase (GLB1); N-sulphoglucosamine sulphohydrolase (SGSH); N-acetyl-alpha-glucosaminidase (NAGLU); iduronate 2-sulfatase (IDS); intracellular cholesterol transporter (NPC1); gigaxonin (GAN); or a combination thereof.
434. The AAV particle of embodiment 431, wherein the antibody or antibody binding fragment binds to:
   (i) a CNS related target, e.g., an antigen associated with a neurological or neurodegenerative disorder, e.g., β-amyloid, APOE, tau, SOD1, TDP-43, huntingtin (HTT), and/or synuclein;
   (ii) a muscular or neuromuscular related target, e.g., an antigen associated with a muscular or neuromuscular disorder; or
   (iii) a neuro-oncology related target, e.g., an antigen associated with a neuro-oncological disorder, e.g., HER2, or EGFR (e.g., EGFRvIII).
435. The AAV particle of embodiment 431, wherein the enzyme comprises a meganuclease, a zinc finger nuclease, a TALEN, a recombinase, integrase, a base editor, a Cas9, or a fragment thereof.
436. The AAV particle of embodiment 431, wherein the component of a gene editing system comprises one or more components of a CRISPR-Cas system.
437. The AAV particle of embodiment 436, wherein the one or more components of the CRISPR-Cas system comprises a Cas9, e.g., a Cas9 ortholog or a Cpf1, and a single guide RNA (sgRNA), optionally wherein:
   (i) the sgRNA is located upstream (5′) of the cas9 enzyme; or
   (ii) the sgRNA is located downstream (3′) of the cas9 enzyme.
438. The AAV particle of embodiment 431, wherein the RNAi agent (e.g., a dsRNA, siRNA, shRNA, pre-miRNA, pri-miRNA, miRNA, stRNA, lncRNA, piRNA, or snoRNA), modulates, e.g., inhibits, expression of, a CNS related gene, mRNA, and/or protein.
439. The AAV particle of embodiment 438, wherein the CNS related gene is chosen from SOD1, MAPT, APOE, HTT, C9ORF72, TDP-43, APP, BACE, SNCA, ATXN1, ATXN3, ATXN7, SCN1A-SCN5A, SCN8A-SCN11A, or a combination thereof.
440. The AAV particle of any one of embodiments 429-439, which comprises a viral genome comprising a promoter operably linked to the nucleic acid sequence encoding the payload.
441. The AAV particle of embodiment 440, wherein the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.
442. The AAV particle of embodiment 440 or 441, wherein the promoter is an EF-1α promoter variant, e.g., a truncated EF-1α promoter.
443. The AAV particle of any one of embodiments 440-442, wherein the promoter comprises the nucleotide sequence of any one of SEQ ID NOs: 19, 20, 22, 23, 27, 28, 30-39 or any one of the sequences provided in Table 12, a nucleotide sequence comprising at least one, two, or three but no more than four modifications, e.g., substitutions (e.g., conservative substitutions), insertions, or deletions, relative to the nucleotide sequence of SEQ ID NOs: 19, 20, 22, 23, 27, 28, 30-39 or any one of the sequences provided in Table 12, or a nucleotide sequence with at least 80% (e.g., 85%, 90%, 95%, 96%, 97%, 98%, or 99%) sequence identity to any one of SEQ ID NOs: 19, 20, 22, 23, 27, 28, 30-39 or any one of the sequences provided in Table 12.
444. The AAV particle of any one of embodiments 440-443, wherein the viral genome further comprises a polyA signal sequence.
445. The AAV particle of any one of embodiments 440-444, wherein the viral genome further comprises an inverted terminal repeat (ITR) sequence.
446. The AAV particle of any one of embodiments 440-445, wherein the viral genome comprises an ITR sequence positioned 5′ relative to the encoded payload.
447. The AAV particle of any one of embodiments 440-446, wherein the viral genome comprises an ITR sequence positioned 3′ relative to the encoded payload.
448. The AAV particle of any one of embodiments 440-447, wherein the viral genome comprises an ITR sequence positioned 5′ relative to the encoded payload and an ITR sequence positioned 3′ relative to the encoded payload.
449. The AAV particle of any one of embodiments 440-448, wherein the viral genome further comprises an enhancer, a Kozak sequence, an intron region, and/or an exon region.
450. The AAV particle of any one of embodiments 440-449, wherein the viral genome further comprises a nucleotide sequence encoding a miR binding site, e.g., a miR binding site that modulates, e.g., reduces, expression of the antibody molecule encoded by the viral genome in a cell or tissue where the corresponding miRNA is expressed.
451. The AAV particle of embodiment 450, wherein the encoded miRNA binding site is complementary, e.g., fully complementary or partially complementary, to a miRNA expressed in a cell or tissue of the DRG, liver, heart, hematopoietic, or a combination thereof.
452. The AAV particle of embodiment 450 or 451, wherein the encoded miR binding site modulates, e.g., reduces, expression of the encoded antibody molecule in a cell or tissue of the DRG, liver, heart, hematopoietic lineage, or a combination thereof.
453. The AAV particle of any one of embodiments 440-452, wherein the viral genome comprises at least 1-5 copies of the encoded miR binding site, e.g., at least 1, 2, 3, 4, or 5 copies.
454. The AAV particle of any one of embodiments 440-453, wherein the viral genome comprises at least 3 copies of an encoded miR binding sites, optionally wherein all three copies comprise the same miR binding site, or at least one, two, three, or all of the copies comprise a different miR binding site.
455. The AAV particle of embodiment 454, wherein the 3 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
456. The AAV particle of any one of embodiments 440-455, wherein the viral genome comprises at least 4 copies of an encoded miR binding site, optionally wherein all four copies comprise the same miR binding site, or at least one, two, three, or all of the copies comprise a different miR binding site.
457. The AAV particle of embodiment 456, wherein the 4 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
458. The AAV particle of any one of embodiments 450-457, wherein the encoded miR binding site comprises a miR122 binding site, a miR183 binding site, a miR-1 binding site, a miR-142-3p, or a combination thereof, optionally wherein:
   (i) the encoded miR122 binding site comprises the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) the encoded miR183 binding site comprises the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 4679; and/or
   (iv) the encoded miR-142-3p binding site comprises the nucleotide sequence of SEQ ID NO: 3674, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3674.
459. The AAV particle of any one of embodiments 440-458, wherein the viral genome comprises an encoded miR122 binding site.
460. The AAV particle of any one of embodiments 440-459, wherein the viral genome comprises at least 1-5 copies, e.g., 1, 2, or 3 copies of a miR122 binding site, optionally wherein each copy is continuous (e.g., not separated by a spacer), or each copy is separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
461. The AAV particle of embodiment 459 or 460, wherein the encoded miR122 binding site comprises the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672.
462. The AAV particle of any one of embodiments 440-461, wherein the viral genome comprises:
(A) (i) a first encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) a first spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (iii) a second encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672; or
(B) (i) a first encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) a first spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA;
   (iii) a second encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (iv) a second spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (v) a third encoded miR122 binding site comprising the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672.
463. The AAV particle of any one of embodiments 440-462, wherein the viral genome comprises an encoded miR183 binding site.
464. The AAV particle of any one of embodiments 440-463, wherein the viral genome comprises at least 1-5 copies, e.g., 1, 2, or 3 copies of a miR183 binding site, optionally wherein each copy is continuous (e.g., not separated by a spacer), or each copy is separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
465. The AAV particle of embodiment 463 or 464, wherein the encoded miR183 binding site comprises the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675.
466. The AAV particle of any one of embodiments 440-465, wherein the viral genome comprises:
(A) (i) a first encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (ii) a first spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (iii) a second encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675; or
(B) (i) a first encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (ii) a first spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA;
   (iii) a second encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (iv) a second spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (v) a third encoded miR183 binding site comprising the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675.
467. The AAV particle of any one of embodiments 440-467, wherein the viral genome comprises an encoded miR122 binding site and a miR-1 binding site.
468. The AAV particle of any one of embodiments 440-468, wherein the viral genome is single stranded or self-complementary.
469. The AAV particle of any one of embodiments 440-469, wherein the viral genome further comprises a nucleotide sequence encoding a Rep protein, e.g., a non-structural protein, wherein the Rep protein comprises a Rep78 protein, a Rep68, Rep52 protein, and/or a Rep40 protein.
470. The AAV particle of embodiment 469, wherein the Rep78 protein, the Rep68 protein, the Rep52 protein, and/or the Rep40 protein are encoded by at least one Rep gene.
471. The AAV particle of any one of embodiments 440-470, wherein the viral genome further comprises a nucleic acid sequence encoding the AAV capsid variant of any one of embodiments 1-420.
472. The AAV particle of any one of embodiments 429-471, which is isolated, e.g., recombinant.
473. A vector comprising a polynucleotide encoding the AAV capsid variant of any one of embodiments 1-420 or 428, the polynucleotide of any one of embodiments 421,422, or 426-428, or a polynucleotide encoding the peptide of any one of embodiments 423-425 or 428.
474. A cell, e.g., a host cell, comprising the AAV capsid variant of any one of embodiments 1-420 or 428, the polynucleotide of any one of embodiments 421, 422, or 426-428, the peptide of any one of embodiments 423-425 or 428, the AAV particle of any one of embodiments 429-472, or the vector of embodiment 273.
475. The cell of embodiment 474, wherein the cell is a mammalian cell or an insect cell.
476. The cell of embodiment 474 or 475, wherein the cell is a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
477. The cell of any one of embodiments 474-476, wherein the cell is a neuron, a sensory neuron, a motor neuron, an astrocyte, a glial cell, or a muscle cell (e.g., a cell of the heart, diaphragm, or quadriceps).
478. A method of making an AAV particle, comprising
   (i) providing a host cell comprising a viral genome; and
   (ii) incubating the host cell under conditions suitable to enclose the viral genome in the AAV capsid variant of any one of embodiments 1-420, or an AAV capsid variant encoded by the polynucleotide of any one of embodiments 421, 422, or 426-428;
   thereby making the AAV particle.
479. The method of embodiment 478, further comprising, prior to step (i), introducing a first nucleic acid molecule comprising the viral genome into the host cell.
480. The method of embodiment 478 or 479, wherein the host cell comprises a second nucleic acid encoding the capsid variant.
481. The method of embodiment 480, wherein the second nucleic acid molecule is introduced into the host cell prior to, concurrently with, or after the first nucleic acid molecule.
482. A pharmaceutical composition comprising the AAV particle of any one of embodiments 429-472, an AAV particle comprising the capsid variant of any one of embodiments 1-420 or 428, an AAV particle comprising the peptide of any one of embodiments 423-425 or 428, and a pharmaceutically acceptable excipient.
483. A method of delivering a payload to a cell or tissue (e.g., a CNS cell or a CNS tissue), comprising administering an effective amount of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428.
484. The method of embodiment 483, wherein the cell is a cell a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
485. The method of embodiment 483 or 484, wherein the cell is a neuron, a sensory neuron, a motor neuron, an astrocyte, or a muscle cell (e.g., a cell of the heart, diaphragm, or quadriceps).
486. The method of any one of embodiments 483-485, wherein the cell or tissue is within a subject.
487. The method of embodiment 486, wherein the subject has, has been diagnosed with having, or is at risk of having a genetic disorder, e.g., a monogenic disorder or a polygenic disorder.
488. The method of embodiment 486 or 487, wherein the subject has, has been diagnosed with having, or is at risk of having a neurological, e.g., a neurodegenerative disorder.
489. The method of embodiment 486 or 487, wherein the subject has, has been diagnosed with having, or is at risk of having a muscular disorder or a neuromuscular disorder.
490. The method of embodiment 486 or 487, wherein the subject has, has been diagnosed with having, or is at risk of having a neuro-oncological disorder.
491. A method of treating a subject having or diagnosed with having a genetic disorder, e.g., a monogenic disorder or a polygenic disorder, comprising administering to the subject an effective amount of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428.
492. A method of treating a subject having or diagnosed with having a neurological disorder, e.g., a neurodegenerative disorder, comprising administering to the subject an effective amount of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428.
493. A method of treating a subject having or diagnosed with having a muscular disorder or a neuromuscular disorder, comprising administering to the subject an effective amount of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428.
494. A method of treating a subject having or diagnosed with having a neuro-oncological disorder, comprising administering to the subject an effective amount of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428.
495. The method of any one of embodiments 487-494, wherein the genetic disorder, neurological disorder, neurodegenerative disorder, muscular disorder, neuromuscular disorder, or neuro-oncological disorder is Huntington's Disease, Amyotrophic Lateral Sclerosis (ALS), Gaucher Disease, Dementia with Lewy Bodies, Parkinson's disease, Spinal Muscular Atrophy, Alzheimer's Disease, or a cancer (e.g., a HER2/neu positive cancer or a glioblastoma).
496. The method of any one of embodiments 491-495, where treating comprises prevention of progression of the disease or disorder in the subject.
497. The method of any one of embodiments 486-496, wherein the subject is a human.
498. The method of any one of embodiments 491-497, wherein the AAV particle is administered to the subject intravenously, via intra-cisterna magna injection (ICM), intracerebrally, intrathecally, intracerebroventricularly, via intraparenchymal administration, or intramuscularly.
499. The method of any one of embodiments 491-498, wherein the AAV particle is administered to the subject via focused ultrasound (FUS), e.g., coupled with the intravenous administration of microbubbles (FUS-MB), or MRI-guided FUS coupled with intravenous administration.
500. The method of any one of embodiments 491-498, wherein the AAV particle is administered to the subject intravenously.
501. The method of any one of embodiments 491-500, wherein administration of the AAV particle results in a decreased presence, level, and/or activity of a gene, mRNA, protein, or combination thereof.
502. The method of any one of embodiments 491-501, wherein administration of the AAV particle results in an increased presence, level, and/or activity of a gene, mRNA, protein, or a combination thereof.
503. The method of any one of embodiments 491-502, wherein the AAV particle is administered at a dose of about 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg.
504. The method of any one of embodiments 491-503, wherein the AAV particle is administered at a dose of about 6.7e10 VG/kg to 6.7e12 VG/kg, about 1.3e11 VG/kg to 3.4e12 VG/kg, or about 2.2e11 VG/kg to 2e12 VG/kg.
505. The method of any one of embodiments 491-277, wherein the AAV particle is administered at a dose of about 4e11 VG/kg to 8e11 VG/kg (e.g., about 6.7e11 VG/kg).
506. The method of any one of embodiments 491-277, wherein the AAV particle is administered at a dose of about 6.7e11 VG/kg.
507. The method of any one of embodiments 491-277, wherein the AAV particle is administered at a dose of about 2e11 VG/kg to 2e13 VG/kg, about 4e11 VG/kg to 1e13 VG/kg, about 6.7e11 VG/kg to about 6e12 VG/kg.
508. The method of any one of embodiments 491-277, wherein the AAV particle is administered at a dose of about 1e12 VG/kg to 5e12 VG/kg (e.g., about 2e12 VG/kg).
509. The method of any one of embodiments 491-277, wherein the AAV particle is administered at a dose of about 2e12 VG/kg.
510. The method of any one of embodiments 491-277, wherein the AAV particle is administered at a dose of about 6.7e11 VG/kg to 6.7e13 VG/kg, about 1.3e12 VG/kg to 3.4e13 VG/kg, or about 2.2e12 VG/kg to 2e13 VG/kg.
511. The method of any one of embodiments 491-277, wherein the AAV particle is administered at a dose of about 4e12 VG/kg to 8e12 VG/kg (e.g., about 6.7e12 VG/kg).
512. The method of any one of embodiments 491-277, wherein the AAV particle is administered at a dose of about 6.7e12 VG/kg.
513. The method of any one of embodiments 491-277, wherein the AAV particle is administered at a dose of about 2e12 VG/kg to 2e14 VG/kg, about 4e12 VG/kg to 1e14 VG/kg, about 6.7e12 VG/kg to about 6e13 VG/kg.
514. The method of any one of embodiments 491-277, wherein the AAV particle is administered at a dose of about 1e13 VG/kg to 5e13 VG/kg (e.g., about 2e13 VG/kg).
515. The method of any one of embodiments 491-514, wherein the AAV particle is administered at a dose of about 2e13 VG/kg.
516. The method of any one of embodiments 503-515, wherein the AAV particle comprises a viral genome which is single stranded.
517. The method of any one of embodiments 503-516, wherein the AAV particle is administered intravenously.
518. The method of any one of embodiments 503-517, wherein administration of the AAV particle at a dose of 2e13 VG/kg is capable of transducing greater than 40% of total cells in brain region chosen from a thalamus, caudate, or putamen, and greater than 20% total cells in a brain region chosen from a entorhinal cortex, auditory cortex, or hippocampus, e.g., when measured by an assay described in Example 10.
519. The method of any one of embodiments 503-518, wherein administration of the AAV particle at a dose of 6.7e12 VG/kg is capable of transducing greater than 20% of total cells in brain region chosen from a thalamus, caudate, putamen, or cerebellum, e.g., when measured by an assay described in Example 10.
520. The method of any one of embodiments 503-519, wherein administration of the AAV particle at a dose of 2e13 VG/kg is capable of transducing greater than 90% SMI311-positive neurons in the thalamus, dentate and spinal cord, e.g., when measured by an assay described in Example 10.
521. The method of any one of embodiments 503-520, wherein administration of the AAV particle at a dose of 2e12 VG/kg is capable of expressing transgene mRNA at a supraphysiological level, e.g., when measured by an assay described in Example 10.
522. The method of any one of embodiments 503-521, wherein administration of the AAV particle at a dose of 2e12 VG/kg is capable of transducing multiple regions of the central nervous system (e.g., one or more regions of the brain and/or spinal cord), e.g., when measured by an assay described in Example 10.
523. The pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428, for use in a method of delivering a payload to a cell or tissue.
524. The pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428, for use in a method of treating a genetic disorder, a neurological disorder, a neurodegenerative disorder, a muscular disorder, a neuromuscular disorder, or a neuro-oncological disorder.
525. The pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428, for use in the manufacture of a medicament.
526. Use of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428, in the manufacture of a medicament.
527. Use of the pharmaceutical composition of embodiment 482, the AAV particle of any one of embodiments 429-472, an AAV particle comprising the capsid variant of any one of embodiments 1-420 or 428, an AAV particle comprising the peptide of any one of embodiments 423-425 or 428, in the manufacture of a medicament for treating a genetic disorder, a neurological disorder, a neurodegenerative disorder, a muscular disorder, a neuromuscular disorder, or a neuro-oncological disorder.
528. A pharmaceutical formulation comprising: (i) an AAV particle or variant thereof, e.g., an AAV particle comprising an AAV capsid variant as described herein (e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments); (ii) a buffering agent; and (iii) a polyether, e.g., one or more polyethers (e.g., glycerol, glycerin, and polyethylene glycol (e.g., low-molecular-weight PEG)).
529. A pharmaceutical formulation comprising:
   (i) the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments;
   (ii) a buffering agent (e.g., a tris base); and
   (iii) a polyether (e.g., glycerol, glycerin, and polyethylene glycol (e.g., low-molecular-weight PEG)).
530. A pharmaceutical formulation comprising: (i) an AAV particle or variant thereof, e.g., an AAV particle comprising an AAV capsid variant as described herein (e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments), (ii) a buffering agent (e.g., a tris base), and (iii) a sugar, e.g., trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose.
531. A pharmaceutical formulation comprising:
   (i) the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments;
   (ii) a buffering agent (e.g., a tris base); and
   (iii) a sugar, e.g., trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose.
532. The pharmaceutical formulation of any one of embodiments 528-531, further comprising a salt.
533. The pharmaceutical formulation of any one of embodiments 528-532, further comprising a surfactant.
534. A pharmaceutical formulation comprising: (a) an AAV particle comprising an AAV capsid variant, e.g., an AAV capsid variant described herein (e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments); (b) a buffering agent; (c) a polyether; (d) a salt; and (e) a surfactant.
535. A pharmaceutical formulation comprising:
   (i) the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments;
   (ii) a buffering agent (e.g., a tris base);
   (iii) a polyether (e.g., glycerol, glycerin, and polyethylene glycol (e.g., low-molecular-weight PEG));
   (iv) a salt (e.g., sodium chloride, potassium chloride, or magnesium chloride); and
   (v) a surfactant (e.g., an ethylene oxide/propylene oxide copolymer (e.g., poloxamers such as Pluronic® F-68 or F-127)), or a zwitterionic surfactant, wherein the concentration of the surfactant, e.g., Pluronic F-68, is between 0.0005-0.0015% (e.g., 0.0008-0.0012%, e.g., 0.001% or about 0.001%)).
536. A pharmaceutical formulation comprising (a) an AAV particle comprising an AAV capsid variant, e.g., an AAV capsid variant described herein (e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments); (b) a buffering agent; (c) a sugar; (d) a salt; and (e) a surfactant.
537. A pharmaceutical formulation comprising:
   (i) the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments;
   (ii) a buffering agent (e.g., a tris base);
   (iii) a sugar (e.g., trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose);
   (iv) a salt (e.g., sodium chloride, potassium chloride, or magnesium chloride); and
   (v) a surfactant (e.g., an ethylene oxide/propylene oxide copolymer (e.g., poloxamers such as Pluronic® F-68 or F-127)), or a zwitterionic surfactant, wherein the concentration of the surfactant, e.g., Pluronic F-68, is between 0.0005-0.0015% (e.g., 0.0008-0.0012%, e.g., 0.001% or about 0.001%)).
538. The pharmaceutical formulation of any one of embodiments 528-537, wherein the buffering agent is capable of maintaining a pH of 7.8-8.4 (e.g., 7.8, 7.9, 8.0, 8.1, 8.2, 8.3, or 8.4).
539. The pharmaceutical formulation of any one of embodiments 528-538, wherein the buffering agent is chosen from a Tris base, Tris HCl, Bis-tris propane (BTP), phosphate-buffered saline (PBS), sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), or MOPS (3-(N-morpholino)propanesulfonic acid).
540. The pharmaceutical formulation of any one of embodiments 528-539, wherein the buffering agent is Tris.
541. The pharmaceutical formulation of any one embodiments 528, 529, 532-535, or 538-450, wherein the polyether is chosen from glycerol, glycerin, or polyethylene glycol (e.g., low-molecular-weight PEG).
542. The pharmaceutical formulation of any one of embodiments 528, 529, 532-535, or 538-541, wherein the polyether is glycerol.
543. The pharmaceutical formulation of any one of embodiments 528, 529, 532-535, or 538-541, wherein the polyether is polyethylene glycol (e.g., low-molecular-weight PEG).
544. The pharmaceutical formulation of any one of embodiments 528, 529, 532-535, or 538-543, wherein the polyether is present at a concentration between:
   (i) about 0.5-5% (e.g., 0.5-3%, 0.5-1.5% (e.g., 0.75-1.25%, e.g., 1%, or about 1%), or 2-3% (e.g., 2.3-2.7%, e.g., 2.5% or about 2.5%));
   (ii) about 0.5-1.5% (e.g., 0.75-1.25%, e.g., 1% or about 1%);
   (iii) about 2-3% (e.g., 2.3-2.7%, e.g., 2.5% or about 2.5%); or
   (iv) about 3-5%.
545. The pharmaceutical formulation of any one of embodiments 528, 529, 532-535, or 538-544, wherein the polyether is present at a concentration of 1%.
546. The pharmaceutical formulation of any one of embodiments 528, 592, 532-535, or 538-544, wherein the polyether is present at a concentration of 2.5%.
547. The pharmaceutical formulation of any one of embodiments 530-533, or 536-540, wherein the sugar is chosen from trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose.
548. The pharmaceutical composition of any one of embodiments 530-533, 536-540, or 457, wherein the sugar is trehalose.
549. The pharmaceutical composition of any one of embodiments 530-533, 536-540, 457, or 458, wherein the sugar is present:
   (i) at a concentration between about 5.5-6.5% (e.g., 5.8-6.2%, e.g., 5.95% or about 5.95%); or
   (ii) at a concentration of 5.95%.
550. The pharmaceutical formulation of any one of embodiments 532-549, wherein the salt is chosen from sodium chloride, potassium chloride, or magnesium chloride.
551. The pharmaceutical formulation of any one of embodiments 532-550, wherein the salt is sodium chloride.
552. The pharmaceutical formulation of any one of embodiments 532-551, wherein the salt is present:
   (i) at a concentration between about 55-70 mM (e.g., 60-65 mM, e.g., 62.5 mM, or about 62.5 mM); or
   (ii) at a concentration of 62.5 mM.
553. The pharmaceutical formulation of any one of embodiments 533-552, wherein the surfactant chosen from:
   (a) an anionic surfactant (e.g., sulfate, sulfonate, phosphate esters, and carboxylates);
   (b) a non-ionic surfactant (e.g., ehoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., poloxamers such as Pluronic® F-68 or F-127), esters of fatty acids and polyhydric alcohols, fatty acid alkanolamides, ethoxylated aliphatic acids, ethoxylated aliphatic alcohols, ethoxylated sorbitol fatty acid esters, ethoxylated glycerides, ethoxylated block copolymers with EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants); and/or
   (c) a zwitterionic surfactant (e.g., alkylamido betaines and amine oxides thereof, alkyl betaines and amine oxides thereof, sulfo betaines, hydroxy sulfo betaines, amphoglycinates, amphopropionates, balanced amphopolycarboxyglycinates, and alkyl polyaminoglycinates).
554. The pharmaceutical formulation of any one of embodiments 533-553, wherein the surfactant is Pluronic F-68.
555. The pharmaceutical formulation of any one of embodiments 533-554, wherein the surfactant is present:
   (i) at a concentration between 0.0005-0.0015% (e.g., 0.0008-0.0012%, e.g., 0.001% or about 0.001%); or
   (ii) at a concentration of 0.001%.
556. The pharmaceutical formulation of any one of embodiments 528, 529, 532-535, 538-547, or 550-555, which comprises at least two, three, or four polyethers.
557. The pharmaceutical formulation of any one of embodiments 530-533, 536-540, or 547-555, which comprises at least two, three, or four sugars.
558. The pharmaceutical formulation of any one of embodiments 532-557, which comprises at least two, three, or four salts.
559. The pharmaceutical formulation of any one of embodiments 533-558, which comprises at least two, three or four surfactants.
560. The pharmaceutical formulation of any one of embodiments 528-559, which exhibits 1, 2, 3, 4, 5, 6, 7, or all of the following properties:
   (a) a pH in the range of between 6-9, for example, a pH of about 6.0, about 6.2, about 6.4, about 6.6, about 6.8, about 7.0, about 7.2, about 7.4, about 7.6, about 7.8, about 8.0, about 8.1, about 8.2, about 8.3, about 8.4, about 8.5, about 8.6, about 8.7, about 8.8, about 8.9, about 9.0, 6.0-8.5, 6.0-7.5, 6.0-7.0, 6.0-6.5, 6.5-9.0, 6.5-8.5, 6.5-8.0, 6.5-7.5, 6.5-7.0, 7.0-9.0, 7.0-8.5, 7.0-8.0, 7.0-7.5, 7.5-9.0, 7.5-8.5, 7.5-8.0, 8.0-9.0, 8.0-8.9, 8.0-8.8, 8.0-8.7, 8.0-8.6, 8.0-8.5, 8.0-8.4, 8.0-8.3, 8.0-8.2, 8.0-8.1, 7.8-8.5, 7.9-8.3, or 7.9-8.2, as assessed by, e.g., a pH meter, as described in Example 9;
   (b) osmolality (mOsm/kg) in the range of between 250-650, for example, 250-650, 250-600, 250-550, 250-500, 250-450, 250-400, 250-350, 250-300, 300-650, 300-600, 300-550, 300-500, 300-450, 300-400, 300-350, 350-650, 350-600, 350-550, 350-500, 350-450, 350-400, 400-650, 400-600, 400-550, 400-500, 400-450, 450-650, 450-600, 450-550, 450-500, 500-650, 500-600, 500-550, 550-650, 550-600, 310-400, 320-400, 330-400, 340-400, 350-400, 360-400, 370-400, 380-400, 390-400, 300-390, 300-380, 300-370, 300-360, 300-350, 300-340, 300-330, 300-320, or 300-310, as assessed by, e.g., an osmometer as described in Example 9. as assessed by, e.g., an osmometer as described in Example 9;
   (c) a viral titer (e.g., TTD-001 titer)>1×10¹² vg/ml, for example, >2×10¹² vg/ml, >4×10¹² vg/ml, >6×10¹² vg/ml, >8×10¹² vg/ml, >1.0×10¹³ vg/ml, >1.5×10¹³ vg/ml, >2.0×10¹³ vg/ml, >2.5×10¹³ vg/ml, >3.0×10¹³ vg/ml, >3.5×10¹³ vg/ml, >4.0×10¹³ vg/ml, >4.5×10¹³ vg/ml, >5.0×10¹³ vg/ml, >5.5×10¹³ vg/ml, >6.0×10¹³ vg/ml, >6.5×10¹³ vg/ml, >7.0×10¹³ vg/ml, >7.5×10¹³ vg/ml, >8.0×10¹³ vg/ml, >8.5×10¹³ vg/ml, >9.0×10¹³ vg/ml, >9.5×10¹³ vg/ml, >1.0×10¹⁴ vg/ml, 1.0×10¹²-1.0×10¹⁴ vg/ml, 2×10¹²-1.0×10¹⁴ vg/ml, 4.0×10¹²-1.0×10¹⁴ vg/ml, 6.0×10¹²-1.0×10¹⁴ vg/ml, 8.0×10¹²-1.0×10¹⁴ vg/ml, 1.0×10¹³-1.0×10¹⁴ vg/ml, 1.5×10¹³-1.0×10¹⁴ vg/ml, 2.0×10¹³-1.0×10¹⁴ vg/ml, 2.5×10¹³-1.0×10¹⁴ vg/ml, 3.0×10¹³-1.0×10¹⁴ vg/ml, 3.5×10¹³-1.0×10¹⁴ vg/ml, 4.0×10¹³-1.0×10¹⁴ vg/ml, 4.5×10¹³-1.0×10¹⁴ vg/ml, 5.0×10¹³-1.0×10¹⁴ vg/ml, 6.0×10¹³-1.0×10¹⁴ vg/ml, 7.0×10¹³-1.0×10¹⁴ vg/ml, 8.0×10¹³-1.0×10¹⁴ vg/ml, 9.0×10¹³-1.0×10¹⁴ vg/ml, 1.0×10¹³-9.0×10¹³ vg/ml, 1.0×10¹³-8.0×10¹³ vg/ml, 1.0×10¹³-7.0×10¹³ vg/ml, 1.0×10¹³-6.0×10¹³ vg/ml, 1.0×10¹³-5.0×10¹³ vg/ml, 1.0×10¹³-4.5×10¹³ vg/ml, 1.0×10¹³-4.0×10¹³ vg/ml, 1.0×10¹³-3.5×10¹³ vg/ml, 1.0×10¹³-3.0×10¹³ vg/ml, 1.0×10¹³-2.5×10¹³ vg/ml, 1.0×10¹³-2.0×10¹³ vg/ml, or 1.0×10¹³-1.5×10¹³ vg/ml, as assessed by, e.g., qPCR as described in Example 9;
   (d) occupancy (% full capsids)≥30%, for example, ≥35%, ≥40%, ≥45%, ≥50%, ≥55%, ≥60%, ≥65%, ≥70%, ≥75%, ≥80%, ≥85%, ≥90%, ≥95%, 30%-90%, 30%-80%, 30%-70%, 30%-65%, 30%-60%, 30%-55%, 30%-50%, 30%-45%, 30%-40%, 30%-35%, 35%-90%, 35%-80%, 35%-75%, 35%-70%, 35%-65%, 35%-60%, 35%-55%, 35%-50%, 35%-45%, 35%-40%, 40%-90%, 40%-80%, 40%-70%, 40%-65%, 40%-60%, 40%-55%, 40%-50%, 40%-45%, 45%-90%, 45%-80%, 45%-70%, 45%-65%, 45%-60%, 45%-55%, 45%-50%, 50%-90%, 50%-80%, 50%-70%, 50%-65%, 50%-60%, 50%-55%, 55%-90%, 55%-80%, 55%-70%, 55%-65%, 55%-60%, 60%-90%, 60%-80%, 60%-70%, 70%-90%, 70%-80%, or 80%-90%, as assessed by, e.g., SEC-MALS as described in Example 9;
   (e) aggregation≤10%, for example, ≤9%, ≤8%, ≤7%, ≤6%, ≤5%, ≤4%, ≤3%, ≤2%, ≤1%, 0-10%, 0-8%, 0-6%, 0-5%, 0-4%, 0-3%, 0-2%, 0-1%, 1-10%, 2-10%, 3-10%, 4-10%, 5-10%, 1-5%, 2-5%, or 3-5%, as assessed by % HMW using size exclusion chromatography (SEC), for example, SEC-FLD/DLS or SEC-MALS, as described in Example 9;
   (f) a viral titer higher than the viral titer supported by a formulation comprising PBS and 0.001% Pluronic F68 (modified PBS formulation) (e.g., higher by at least 50%, at least 100%, at least 250%, at least 500%, at least 750%, at least 1000% (e.g., one order of magnitude), at least 2000%, at least 4000%, at least 6000%, at least 8000%, at least 10,000% (e.g., two orders of magnitude), at least 25,000%, at least 50,000%, 500-50,000%, 1000-50,000%, 2500-50,000%, 5000-50,000%, 7500-50,000%, 10,000-50,000%, 25,000-50,000%, 500-25,000%, 1000-25,000%, 2500-25,000%, 5000-25,000%, 7500-25,000%, 10,000-25,000%, 500-10,000%, 1000-10,000%, 2500-10,000%, 5000-10,000%, 7500-10,000%, 500-7500%, 1000-7500%, 2500-7500%, 5000-7500%, 500-5000%, 1000-7500%, or 2500-5000%), as assessed by, e.g., qPCR as described in Example 9;
   (g) occupancy (% full capsids) higher than the occupancy supported by a formulation comprising PBS and 0.001% Pluronic F68 (modified PBS formulation) (e.g., higher relative to the modified PBS formulation by, e.g., ≥10%, ≥15%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90%, ≥95%, ≥100% (e.g., at least 2-times higher), ≥125%, ≥150%, 10-150%, 20-150%, 30-150%, 40-150%, 50-150%, 60-150%, 70-150%, 80-150%, 90-150%, 100-150%, 125-150%, 10-125%, 20-125%, 30-125%, 40-125%, 50-125%, 60-125%, 70-125%, 80-125%, 90-125%, 100-125%, 10-100%, 20-100%, 30-100%, 40-100%, 50-100%, 60-100%, 70-100%, 80-100%, 90-100%, 10-75%, 20-75%, 30-75%, 40-75%, 50-75%, 60-75%, 10-50%, 20-50%, 30-50%, 40-50%, or 10-25%), as assessed by, e.g., SEC-MALS as described in Example 9; and/or
   (h) aggregation less than the aggregation observed with a formulation comprising PBS and 0.001% Pluronic F68 (modified PBS formulation) after multiple freeze thaw cycles (e.g., 1, 2, 3, 4, 5, 6, or more freeze thaw cycles), e.g., aggregation less than that observed with the modified PBS formulation after 1, 2, 3, 4, 5, 6, or more freeze thaw cycles by at least 20%, 25%, 30%, 35%, 40%, 45%, 50%, 55%, 60%, 65%, 70%, 75%, 80%, 85%, 90%, 20-90%, 30-90%, 40-90%, 50-90%, 60-90%, 70-90%, 80-90%, 20-80%, 30-80%, 40-80%, 50-80%, 60-80%, 70-80%, 20-70%, 30-70%, 40-70%, 50-70%, 60-70%, 20-60%, 30-60%, 40-60%, 50-60%, 20-50%, 30-50%, 40-50%, 20-40%, 30-40%, or 20-30%, as assessed by % HMW using size exclusion chromatography (SEC), for example, SEC-FLD/DLS or SEC-MALS, as described in Example 9.
561. The pharmaceutical formulation of any one of embodiments 528, 529, 532-535, 538-547, 550-560, which comprises (a) an AAV particle or variant thereof, e.g., an AAV particle comprising an AAV capsid variant as described herein, e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments; (b) Tris; (c) glycerol; (d) sodium chloride; and (e) Pluronic F-68.
562. The pharmaceutical formulation of any one of embodiments 530-533, 536-540, 547-555, or 557-560, which comprises (a) an AAV particle or variant thereof, e.g., an AAV particle comprising an AAV capsid variant as described herein, e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments; (b) Tris; (c) trehalose; (d) sodium chloride; and (e) Pluronic F-68.
563. The pharmaceutical formulation of embodiment 561 or 562, wherein the concentration of Tris is between 10-30 mM (e.g., 15-25 mM, e.g., 20 mM or about 20 mM).
564. The pharmaceutical formulation of embodiment 561 or 563, wherein the concentration of glycerol is between 0.5-1.5% (e.g., 0.75-1.25%, e.g., 1% or about 1%).
565. The pharmaceutical formulation of embodiment 561 or 563, wherein the concentration of glycerol is between 2-3% (e.g., 2.3-2.7%, e.g., 2.5% or about 2.5%).
566. The pharmaceutical formulation of embodiment 562 or 563, wherein the concentration of trehalose is between 5.5-6.5% (e.g., 5.8-6.2%, e.g., 5.95% or about 5.95%).
567. The pharmaceutical formulation of any one of embodiments 561-566, wherein the concentration of sodium chloride is between 55-70 mM (e.g., 60-65 mM, e.g., 62.5 mM or about 62.5 mM).
568. The pharmaceutical formulation of embodiment 561-567, wherein the concentration of Pluronic F-68 is between 0.0005-0.0015% (e.g., 0.0008-0.0012%, e.g., 0.001% or about 0.001%).
569. A pharmaceutical formulation comprising:
   (i) the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments;
   (ii) a buffering agent, e.g., a Tris base, capable of maintaining a pH of 7.8-8.4 (e.g., 7.8, 7.9, 8.0, 8.1, 8.2, 8.3, or 8.4), wherein the concentration of the buffering agent is between 10-30 mM (e.g., 15-25 mM, e.g., 20 mM or about 20 mM);
   (iii) a polyether, e.g., glycerol, glycerin, or polyethylene glycol (e.g., low-molecular-weight PEG), wherein the concentration of the polyether, e.g., glycerol, is between 0.5-5% (e.g., 0.5-3%, 0.5-1.5% (e.g., 0.75-1.25%, e.g., 1%, or about 1%), or 2-3% (e.g., 2.3-2.7%, e.g., 2.5% or about 2.5%)); (iv) a salt, e.g., sodium chloride, potassium chloride, or magnesium chloride, wherein the concentration of the salt, e.g., sodium chloride, is between 55-70 mM (e.g., 60-65 mM, e.g., 62.5 mM or about 62.5 mM); and
   (v) a surfactant, e.g., an anionic surfactant, a non-ionic surfactant (e.g., an ethylene oxide/propylene oxide copolymer (e.g., poloxamers such as Pluronic® F-68 or F-127)), or a zwitterionic surfactant, wherein the concentration of the surfactant, e.g., Pluronic F-68, is between 0.0005-0.0015% (e.g., 0.0008-0.0012%, e.g., 0.001% or about 0.001%)).
570. A pharmaceutical formulation comprising (a) an AAV particle or variant thereof, e.g., an AAV particle comprising an AAV capsid variant as described herein, e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments; (b) 20 mM or about 20 mM Tris; (c) 1% or about 1% glycerol; (d) 62.5 mM or about 62.5 mM sodium chloride; and (e) 0.001% or about 0.001% Pluronic F-68.
571. A pharmaceutical formulation comprising:
   (i) the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments;
   (ii) a buffering agent, e.g., a Tris base, capable of maintaining a pH of 7.8-8.4 (e.g., 7.8, 7.9, 8.0, 8.1, 8.2, 8.3, or 8.4), wherein the concentration of the buffering agent is between 10-30 mM (e.g., 15-25 mM, e.g., 20 mM or about 20 mM);
   (iii) a sugar, e.g., trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose, wherein the concentration of the sugar, e.g., trehalose, is between 5.5-6.5% (e.g., 5.8-6.2%, e.g., 5.95% or about 5.95%);
   (iv) a salt, e.g., sodium chloride, potassium chloride, or magnesium chloride, wherein the concentration of the salt, e.g., sodium chloride, is between 55-70 mM (e.g., 60-65 mM, e.g., 62.5 mM or about 62.5 mM); and
   (v) a surfactant, e.g., an anionic surfactant, a non-ionic surfactant (e.g., an ethylene oxide/propylene oxide copolymer (e.g., poloxamers such as Pluronic® F-68 or F-127), or a zwitterionic surfactant, wherein the concentration of the surfactant, e.g., Pluronic F-68, is between 0.0005-0.0015% (e.g., 0.0008-0.0012%, for example, 0.001% or about 0.001%)).
572. A pharmaceutical formulation comprising (a) an AAV particle or variant thereof, e.g., an AAV particle comprising an AAV capsid variant as described herein, e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments; (b) 20 mM or about 20 mM Tris; (c) 2.5% or about 2.5% glycerol; (d) 62.5 mM or about 62.5 mM sodium chloride; and (e) 0.001% or about 0.001% Pluronic F-68.
573. A pharmaceutical formulation comprising (a) an AAV particle or variant thereof, e.g., an AAV particle comprising an AAV capsid variant as described herein, e.g., the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments; (b) 20 mM or about 20 mM Tris; (c) 5.95% or about 5.95% trehalose; (d) 62.5 mM or about 62.5 mM sodium chloride; and (e) 0.001% or about 0.001% Pluronic F-68.
574. The pharmaceutical formulation of any one of embodiments 528-573, wherein the formulation has a pH of between 7.8-8.4 (e.g., 7.8, 7.9, 8.0, 8.1, 8.2, 8.3, or 8.4).
575. The pharmaceutical formulation of any one of embodiments 528-574, wherein the formulation has an osmolality of between 250-550 mOsm/kg (e.g., between 300-550, 400-550, 250-500, 300-500, 250-450, 300-450, or 300-400).
576. The pharmaceutical formulation of any one of embodiments 528-575, wherein the formulation remains stable after storage at −80° C., 2-8° C., or room temperature for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), as reflected by a change (e.g., an increase or decrease) in osmolality, viral titer, occupancy, and/or aggregation, relative to baseline (e.g., prior to storage) of less than 50% (e.g., less than 40%, 30%, 20%, 10%, or 5%), e.g., as measured by an assay described herein.
577. The pharmaceutical formulation of any one of embodiments 528-576, wherein: (a) the ratio of VP3 protein to VP2 protein (VP3:VP2) of the AAV capsids of the AAV particle is about 25-35:2 (e.g., 25-34:2, 25-33:2, 25-32:2, 25-30:2, 25:2, 26:2, 27:2, 28:2, 29:2, 30:2, 31:2, 32:2, 33:2, 34:2, or 35:2),
   (b) the ratio of VP3 protein to VP1 protein (VP3:VP1) of the AAV capsids of the AAV particle is about 25-35:1 (e.g., 25-34:1, 25-33:1, 25-32:1, 25-30:1, 25:1, 26:1, 27:1, 28:1, 29:1, 30:1, 31:1, 32:1, 33:1, 34:1, or 35:1), and/or
   (c) the ratio of VP3 protein to VP2 and VP1 proteins (VP3:VP2:VP1) of the AAV capsids of the AAV particle is about 25-35:2:1 (e.g., 25-34:2:1, 25-33:2:1, 25-32:2:1, 25-30:2:1, 25:2:1, 26:2:1, 27:2:1, 28:2:1, 29:2:1, 30:2:1, 31:2:1, 32:2:1, 33:2:1, 34:2:1, or 35:2:1), when the pharmaceutical formulation is stored at −80° C. for 90 days or more, e.g., when assessed by capillary gel electrophoresis-sodium dodecyl sulfate (e.g., as described in Example 9).
578. The pharmaceutical formulation of any one of embodiments 528-577, wherein the ratio of VP3 protein to VP2 protein (e.g., VP3:VP2), VP3 protein to VP1 protein (VP3:VP1), or VP3 protein to VP2 and VP1 proteins (VP3:VP2:VP1) of the AAV capsids of the AAV particle does not differ from a reference ratio (e.g., baseline ratio, such as the ratio at time 0) by more than about 25% (e.g., about 20%, about 15%, about 10%, about 5%, about 1%, 1-25%, 1-20%, 1-15%, 1-10%, 1-5%, 5-25%, 5-20%, 5-15%, 5-10%, 10-25%, 10-20%, 10-15%, 15-25%, 15-20%, or 20-25%) when the pharmaceutical formulation is stored at −80° C. for 90 days or more, e.g., when assessed by capillary gel electrophoresis-sodium dodecyl sulfate (e.g., as described in Example 9).
579. The pharmaceutical formulation of any one of embodiments 528-578, wherein the purity of AAV capsids of the AAV particle is at least 95% (e.g., at least 96%, at least 97%, at least 98%, at least 99%, or 100%) when the pharmaceutical formulation is stored at −80° C. for 90 days or more, e.g., when assessed by capillary gel electrophoresis-sodium dodecyl sulfate (e.g., as described in Example 9).
580. The pharmaceutical formulation of any one of embodiments 528-579, wherein the purity of AAV capsids of the AAV particle does not differ from a reference value (e.g., baseline value, such as the value at time 0) by more than about 5% (e.g., about 4%, about 3%, about 2%, about 1%) when the pharmaceutical formulation is stored at −80° C. for 90 days or more, e.g., when assessed by capillary gel electrophoresis-sodium dodecyl sulfate (e.g., as described in Example 9).
581. The pharmaceutical formulation of any one of embodiments 528-580, wherein:
   (a) the pI value (e.g., central pI value) of the AAV capsids of the AAV particle is in the range of 5.8-6.5 (e.g., 5.8-6.4, 5.8-6.3, 5.8-6.2, 5.8-6.1, 5.9-6.4, 5.9-6.3, 5.9-6.2, 5.9-6.1, 6.0-6.4, 6.0-6.3, 6.0-6.2, 6.0-6.1, 6.1-6.4, 6.1-6.3, 6.1-6.2, 6.2-6.4, 6.2-6.3, 6.2-6.2, or 6.3-6.4) when the pharmaceutical formulation is stored at −80° C. for 90 days or more, e.g., when assessed by cIEF (e.g., as described in Example 9),
   (b) the pI value (e.g., central pI value) of the AAV capsids of the AAV particle does not differ from a reference value (e.g., baseline pI value (e.g., central pI value), such as the pI at time 0) by more than about 10% (e.g., about 9%, about 8%, about 7%, about 6%, about 5%, about 4%, about 3%, about 2%, about 1%, 1-10%, 1-8%, 1-6%, 1-5%, 1-4%, 1-3%, or 1-2%) when the pharmaceutical formulation is stored at −80° C. for 90 days or more, e.g., when assessed by cIEF (e.g., as described in Example 9),
   (c) the pI value (e.g., central pI value) of the AAV capsids of the AAV particle is in the range of 5.9-6.2 (e.g., 5.9-6.0, 5.9-6.1, 6.0-6.1, 6.0-6.2, 6.1-6.2, 5.9, 6.0, 6.1, or 6.2) when the pharmaceutical formulation is stored at 2-8° C. for 90 days or more, e.g., when assessed by cIEF (e.g., as described in Example 9), and/or
(d) the pI value (e.g., central pI value) of the AAV capsids of the AAV particle does not differ from a reference value (e.g., baseline pI value (e.g., central pI value), such as the pI at time 0) by more than about 10% (e.g., about 9%, about 8%, about 7%, about 6%, about 5%, about 4%, about 3%, about 2%, about 1%, 1-10%, 1-8%, 1-6%, 1-5%, 1-4%, 1-3%, or 1-2%) when the pharmaceutical formulation is stored at 2-8° C. for 90 days or more, e.g., when assessed by cIEF (e.g., as described in Example 9).
582. The pharmaceutical formulation of any one of embodiments 528-581, wherein the AAV viral titer is at least 5×1012 vg/ml (e.g., at least 6×1012 vg/ml, at least 7×1012 vg/ml, at least 8×1012 vg/ml, at least 9×1012 vg/ml, at least 1×1013 vg/ml, at least 2×1013 vg/ml, at least 3×1013 vg/ml, at least 4×1013 vg/ml, at least 5×1013 vg/ml, at least 6×1013 vg/ml, at least 7×1013 vg/ml, at least 8×1013 vg/ml, at least 9×1013 vg/ml, at least 1×1014 vg/ml, 5×1012 vg/ml to 1×1014 vg/ml, 5×1012 vg/ml to 5×1013 vg/ml, 1×1013 vg/ml to 1×1014 vg/ml, 1×1013 vg/ml to 5×1013 vg/ml, or 5×1012 vg/ml to 5×1013 vg/ml) when the pharmaceutical formulation is (a) stored at 2-8° C. for at least 14 days (e.g., 14 days, 30 days, 60 days, or 90 days), (b) stored at −80° C. for at least 14 days (e.g., 14 days, 30 days, 60 days, or 90 days), (c) stored at 2-8° C. for at least 14 days (e.g., 14 days, 30 days, 60 days, or 90 days), or (d) subjected to 1-6 freeze thaw cycles (e.g., 1-5, 1-4, 1-3, 1-2, 2-6, 2-5, 2-4, 2-3, 3-6, 3-5, 3-4, 4-6, or 4-5 freeze thaw cycles), e.g., when assessed by qPCR as described in Example 9.
583. The pharmaceutical formulation of any one of embodiments 528-582, wherein:
   (a) the % high molecular weight (% HMW) aggregates in the pharmaceutical formulation is about 10% or less (e.g., 9%, 8%, 7%, 6%, 5%, 4%, 3%, 2%, 1%, 1-10%, 1-9%, 1-8%, 1-7%, 1-6%, 1-5%, or 1-4%) when the pharmaceutical formulation is stored at −80° C. for at least 30 days (e.g., 30 days, 60 days, or 90 days), e.g., when assessed by size exclusion chromatography (e.g., as described in Example 9),
   (b) the % HMW aggregates in the pharmaceutical formulation does not differ from a reference value (e.g., baseline % HMW, such as % HMW at time 0) by more than about 30% (e.g., about 25%, about 20%, about 15%, about 10%, or about 5%) when the pharmaceutical formulation is stored at −80° C. for at least 30 days (e.g., 30 days, 60 days, or 90 days), e.g., when assessed by size exclusion chromatography (e.g., as described in Example 9),
   (c) the % HMW aggregates in the pharmaceutical formulation is about 5% or less (e.g., 4%, 3%, 2%, 1%, 1-5%, 1-4%, or 1-3%) when the pharmaceutical formulation is stored at room temperature for at least 30 days (e.g., 30 days, 60 days, or 90 days), e.g., when assessed by size exclusion chromatography (e.g., as described in Example 9),
   (d) the % HMW aggregates in the pharmaceutical formulation does not differ from a reference value (e.g., baseline % HMW, such as % HMW at time 0) by more than about 30% (e.g., about 25%, about 20%, about 15%, about 10%, or about 5%) when the pharmaceutical formulation is stored at room temperature for at least 30 days (e.g., 30 days, 60 days, or 90 days), e.g., when assessed by size exclusion chromatography (e.g., as described in Example 9),
   (e) the % HMW aggregates in the pharmaceutical formulation is about 5% or less (e.g., 4%, 3%, 2%, 1%, 1-5%, 1-4%, or 1-3%) when the pharmaceutical formulation is stored at 2-8° C. for at least 90 days (e.g., 90 days), e.g., when assessed by size exclusion chromatography (e.g., as described in Example 9),
   (f) the % HMW aggregates in the pharmaceutical formulation does not differ from a reference value (e.g., baseline % HMW, such as % HMW at time 0) by more than about 30% (e.g., about 25%, about 20%, about 15%, about 10%, or about 5%) when the pharmaceutical formulation is stored at 2-8° C. for at least 90 days (e.g., 90 days), e.g., when assessed by size exclusion chromatography (e.g., as described in Example 9),
   (g) the % HMW aggregates in the pharmaceutical formulation is about 5% or less (e.g., 4%, 3%, 2%, 1%, 1-5%, 1-4%, or 1-3%) when subjected to one or more (e.g., 1, 2, 3, 4, 5, 6, or more) freeze thaw cycles (e.g., −80° C. to 25° C. freeze thaw), e.g., when assessed by size exclusion chromatography (e.g., as described in Example 9), and/or
   (h) the % HMW aggregates in the pharmaceutical formulation does not differ from a reference value (e.g., baseline % HMW, such as % HMW at time 0) by more than about 20% (e.g., about 15%, about 10%, or about 5%) when subjected to one or more (e.g., 1, 2, 3, 4, 5, 6, or more) freeze thaw cycles (e.g., −80° C. to 25° C. freeze thaw), e.g., when assessed by size exclusion chromatography (e.g., as described in Example 9).
584. The pharmaceutical formulation of any one of embodiments 528-583, wherein:
   (a) the % full capsids of the AAV particle is at least 45% (e.g., at least 50%, at least 60%, at least 70%, 45-70%, 50-70%, 45-60%, or 45-55%) when the pharmaceutical formulation is stored at −80° C. for at least 7 days (e.g., 7 days, 14 days, or 30 days), e.g., when assessed as described in Example 9,
   (b) the % full capsids of the AAV particle does not differ from a reference value (e.g., baseline % full capsids at time 0) by more than about 10% (e.g., about 5%, about 3%, about 1%, 1-10%, 1-5%, 1-3%, or 5-10%) when the pharmaceutical formulation is stored at −80° C. for at least 7 days (e.g., 7 days, 14 days, or 30 days), e.g., when assessed as described in Example 9,
   (c) the % full capsids of the AAV particle is at least 35% (e.g., at least 40%, at least 45%, at least 50%, at least 60%, at least 70%, 35-70%, 35-60%, 35-50%, 35-45%, 40-70%, 40-60% 40-50%, 40-45%, 45-70%, 45-60%, 45-50%, 50-70%, or 50-60%) when the pharmaceutical formulation is stored at 2-8° C. for at least 7 days (e.g., 7 days, 14 days, or 30 days), e.g., when assessed as described in Example 9,
   (d) the % full capsids of the AAV particle does not differ from a reference value (e.g., baseline % full capsids at time 0) by more than about 25% (e.g., about 20%, about 15%, about 10%, or about 5%) when the pharmaceutical formulation is stored at 2-8° C. for at least 7 days (e.g., 7 days, 14 days, or 30 days), e.g., when assessed as described in Example 9,
   (e) the % full capsids of the AAV particle is at least 30% (e.g., at at least 35%, least 40%, at least 45%, at least 50%, at least 60%, at least 70%, 30-70%, 30-60%, 30-50%, 30-40%, 35-70%, 35-60%, 35-50%, 35-40%, 40-70%, 40-60% 40-50%, 45-70%, 45-60%, 45-50%, 50-70%, or 50-60%) when the pharmaceutical formulation is stored at room temperature for at least 7 days (e.g., 7 days, 14 days, or 30 days), e.g., when assessed as described in Example 9, and/or
   (f) the % full capsids of the AAV particle does not differ from a reference value (e.g., baseline % full capsids at time 0) by more than about 40% (e.g., about 35%, about 30%, about 25%, about 20%, about 15%, about 10%, or about 5%) when the pharmaceutical formulation is stored at room temperature for at least 7 days (e.g., 7 days, 14 days, or 30 days), e.g., when assessed as described in Example 9.
585. The pharmaceutical formulation of any one of embodiments 528-584, wherein the AAV particle of any one of embodiments 429-472 of the enumerated embodiments, an AAV particle comprising the capsid variant of any one of embodiments 1-420 or 428 of the enumerated embodiments, an AAV particle comprising the peptide of any one of embodiments 423-425 or 428 of the enumerated embodiments; or the AAV particle of any one of embodiments 124-152 of the additional enumerated embodiments, an AAV particle comprising the capsid variant of any one of embodiments 1-102 or 108 of the additional enumerated embodiments, or an AAV particle comprising the peptide of embodiment 107 of the additional enumerated embodiments.
586. The pharmaceutical formulation of any one of embodiments 528-585, wherein the AAV particle, or variant thereof, comprises an AAV capsid variant comprising:
   (i) the amino acid sequence of any one of SEQ ID NOs: 5, 8, 3636-3647, or a sequence with at least 80% (e.g., at least about 85, 90, 95, 96, 97, 98, or 99%) sequence identity thereto;
   (ii) an amino acid sequence comprising at least one, two or three modifications, but not more than 30, 20 or 10 modifications relative to the amino acid sequence of any one of SEQ ID NOs: 5, 8, 3636-3647;
   (iii) an amino acid sequence comprising at least one, two or three, but not more than 30, 20 or 10 different amino acids relative to the amino acid sequence of any one of SEQ ID NOs: 5, 8, 3636-3647.
587. The pharmaceutical formulation of any one of embodiments 528-586, wherein the AAV particle, or variant thereof, comprises an AAV capsid variant comprising:
   (i) the amino acid sequence of SEQ ID NO: 5, 8, 3636, or a sequence with at least 80% (e.g., at least about 85, 90, 95, 96, 97, 98, or 99%) sequence identity thereto;
   (ii) an amino acid sequence comprising at least one, two or three modifications, but not more than 30, 20 or 10 modifications relative to the amino acid sequence of SEQ ID NO: 5, 8, 3636;
   (iii) an amino acid sequence comprising at least one, two or three, but not more than 30, 20 or 10 different amino acids relative to the amino acid sequence of SEQ ID NOs: 5, 8, 3636.
588. The pharmaceutical formulation of any one of embodiments 528-587, which is administered intravenously to a subject (e.g., a human), e.g., in a high volume (e.g., a volume comprising more than 100 mL) or a low volume (e.g., a volume comprising 100 mL or a volume comprising no more than 100 mL).
589. The pharmaceutical formulation of any one of embodiments 528-588, wherein the AAV particle is present at a dose of about 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg.
590. The pharmaceutical formulation of any one of embodiments 528-589, wherein the AAV particle is present at a dose of about 6.7e10 VG/kg to 6.7e12 VG/kg, about 1.3e11 VG/kg to 3.4e12 VG/kg, or about 2.2e11 VG/kg to 2e12 VG/kg.
591. The pharmaceutical formulation of any one of embodiments 528-590, wherein the AAV particle is present at a dose of about 4e11 VG/kg to 8e11 VG/kg (e.g., about 6.7e11 VG/kg).
592. The pharmaceutical formulation of any one of embodiments 528-591, wherein the AAV particle is present at a dose of about 6.7e11 VG/kg.
593. The pharmaceutical formulation of any one of embodiments 528-592, wherein the AAV particle is present at a dose of about 2e11 VG/kg to 2e13 VG/kg, about 4e11 VG/kg to 1e13 VG/kg, about 6.7e11 VG/kg to about 6e12 VG/kg.
594. The pharmaceutical formulation of any one of embodiments 528-593, wherein the AAV particle is present at a dose of about 1e12 VG/kg to 5e12 VG/kg (e.g., about 2e12 VG/kg).
595. The pharmaceutical formulation of any one of embodiments 528-594, wherein the AAV particle is present at a dose of about 2e12 VG/kg.
596. The pharmaceutical formulation of any one of embodiments 528-595, wherein the AAV particle is present at a dose of about 6.7e11 VG/kg to 6.7e13 VG/kg, about 1.3e12 VG/kg to 3.4e13 VG/kg, or about 2.2e12 VG/kg to 2e13 VG/kg.
597. The pharmaceutical formulation of any one of embodiments 528-596, wherein the AAV particle is present at a dose of about 4e12 VG/kg to 8e12 VG/kg (e.g., about 6.7e12 VG/kg).
598. The pharmaceutical formulation of any one of embodiments 528-597, wherein the AAV particle is present at a dose of about 6.7e12 VG/kg.
599. The pharmaceutical formulation of any one of embodiments 528-598, wherein the AAV particle is present at a dose of about 2e12 VG/kg to 2e14 VG/kg, about 4e12 VG/kg to 1e14 VG/kg, about 6.7e12 VG/kg to about 6e13 VG/kg.
600. The pharmaceutical formulation of any one of embodiments 528-599, wherein the AAV particle is administered at a dose of about 1e13 VG/kg to 5e13 VG/kg (e.g., about 2e13 VG/kg).
601. The pharmaceutical formulation of any one of embodiments 528-600, wherein the AAV particle is administered at a dose of about 2e13 VG/kg.
602. The pharmaceutical formulation of any one of embodiments 581-601, wherein the AAV particle comprises a viral genome which is single stranded.
603. The pharmaceutical formulation of any one of embodiments 528-602, which is formulated for intravenous administration.
604. A method of treating a subject having or diagnosed with having a genetic disorder (e.g., a monogenic disorder or a polygenic disorder), a neurological disorder (e.g., a neurodegenerative disorder), a muscular disorder, a neuromuscular disorder, or a neuro-oncological disorder, comprising administering to the subject an effective amount of the pharmaceutical formulation of any one of embodiments 528-603, optionally wherein the pharmaceutical formulation is administered to the subject intravenously, e.g., in a high volume (e.g., a volume comprising more than 100 mL) or a low volume (e.g., a volume comprising 100 mL or a volume comprising no more than 100 mL).
605. The pharmaceutical formulation of any one of embodiments 528-603, for use in a method of treating a genetic disorder (e.g., a monogenic disorder or a polygenic disorder), a neurological disorder (e.g., a neurodegenerative disorder), a muscular disorder, a neuromuscular disorder, or a neuro-oncological disorder, optionally wherein the pharmaceutical formulation is suitable for intravenous administration, e.g., in a high volume (e.g., a volume comprising more than 100 mL) or a low volume (e.g., a volume comprising 100 mL or a volume comprising no more than 100 mL).
606. Use of pharmaceutical formulation of any one of embodiments 528-603, in the manufacture of a medicament, optionally wherein the pharmaceutical formulation is suitable for intravenous administration, e.g., in a high volume (e.g., a volume comprising more than 100 mL) or a low volume (e.g., a volume comprising 100 mL or a volume comprising no more than 100 mL).
607. Use of pharmaceutical formulation of any one of embodiments 528-603, in the manufacture of a medicament for treating a genetic disorder (e.g., a monogenic disorder or a polygenic disorder), a neurological disorder (e.g., a neurodegenerative disorder), a muscular disorder, a neuromuscular disorder, or a neuro-oncological disorder, optionally wherein the pharmaceutical formulation is suitable for intravenous administration, e.g., in a high volume (e.g., a volume comprising more than 100 mL) or a low volume (e.g., a volume comprising 100 mL or a volume comprising no more than 100 mL).
608. The method of embodiment 604, the pharmaceutical composition for use of embodiment 605, or the use of embodiment 606 or 607, which is formulated for intravenous administration.
609. A peptide comprising an amino acid sequence comprising the formula [N1]-[N2] according to any one of embodiments 1-13, 17-28, 34-41, 46-68, or 115-118.
610. A peptide comprising an amino acid sequence comprising the formula [A][B] according to any one of embodiments 119-127, 131-141, 147-153, 159-177, or 223-226.
611. A peptide comprising an amino acid sequence comprising the formula X1-X2-X3-X4-X5-X6-X7-X8-X9-X10-X11-X12-X13-X14-X15-X16-X17-X18-X19, according to any one of embodiments 252-254.
612. A peptide comprising an amino acid sequence comprising the formula [N1]-[N2], according to any one of embodiments 259-272, 278, 284, or 286.
613. A peptide comprising an amino sequence comprising the formula [B]-[C], according to any one of embodiments 291-302, 308, 314, or 316.
614. A peptide comprising an amino acid sequence comprising positions X1-X2-X3-X4-X5-X6-X7-X8-X9, according to embodiment 321.
615. The peptide of any one of embodiments 423-425 or 609-614, which is fused or coupled, e.g., conjugated, to an active agent, e.g., a therapeutic agent or a diagnostic agent.
616. The peptide of any one of embodiments 423-425 or 609-614, wherein at least 1-5, e.g., at least 1, 2, 3, 4, or 5, peptides are fused or coupled, e.g., conjugated, to an active agent, e.g., a therapeutic agent or a diagnostic agent.
617. The peptide of embodiment 616, wherein the at least 1-5, e.g., at least 1, 2, 3, 4, or 5, peptides comprise the same amino acid sequence.
618. The peptide of embodiment 616, wherein the at least 1-5, e.g., at least 1, 2, 3, 4, or 5, peptides comprise different amino acid sequences.
619. The peptide of any one of embodiments 615-618, wherein the at least 1-5, e.g., at least 1, 2, 3, 4, or 5, peptides are present in tandem (e.g., connected directly or indirectly via a linker) or in a multimeric configuration.
620. The peptide of any one of embodiments 423-425 or 609-619, wherein the peptide comprises an amino acid sequence of at least 3, 4, 5, 6, 7, 8, 9, 10, 11, 12, 15, 20, 25, 30, or 35 amino acids in length.
621. The peptide of any one of embodiments 615-620, wherein the active agent is or comprises a therapeutic agent chosen from a protein (e.g., an enzyme), an antibody molecule, a nucleic acid molecule (e.g., an RNAi agent), or a small molecule.
622. The peptide of any one of embodiments 615-620, wherein the active agent is or comprises a ribonucleic acid complex (e.g., a Cas9/gRNA complex), a plasmid, a closed-end DNA, a circ-RNA, or an mRNA.
623. The peptide of any one of embodiments 615-620, wherein the active agent is an RNAi agent.
624. The peptide of embodiment 623, wherein the RNAi agent is a dsRNA, a siRNA, a shRNA, a pre-miRNA, a pri-miRNA, a miRNA, a stRNA, a lncRNA, a piRNA, an antisense oligonucleotide agent (ASO), or a snoRNA, optionally wherein the RNAi agent is an siRNA or an ASO, which further optionally comprises at least one modified nucleotide.
625. The peptide of any one of embodiments 615-624, wherein the active agent modulates, e.g., inhibits, decreases or increases, expression of, a CNS related gene, mRNA, and/or protein.
626. The peptide of any one of embodiments 615-620, wherein the active agent is a diagnostic agent is or comprises an imaging agent (e.g., a protein or small molecule compound coupled to a detectable moiety).
627. The peptide of any one of embodiments 615-626, wherein the peptide covalently linked, e.g., directly or indirectly via a linker, to the active agent.
628. The peptide of any one of embodiments 615-627, wherein the peptide is conjugated to the active agent via a linker.
629. The peptide of embodiment 628, wherein the linker is a cleavable linker or a non-cleavable linker.
630. The peptide of embodiment 628, wherein the cleavable linker is a pH sensitive linker or an enzyme sensitive linker.
631. The peptide of embodiment 629 or 630, wherein:
   (i) the pH sensitive linker comprises a hydrazine/hydrazone linker or a disulfide linker;
   (ii) the enzyme sensitive linker comprises a peptide based linker, e.g., a peptide linker sensitive to a protease (e.g., a lysosomal protease); or a beta-glucuronide linker; or
   (iii) the non-cleavable linker is a linker comprising a thioether group or a maleimidocaproyl group.
632. The peptide of any one of embodiments 615-631, wherein:
   (i) the peptide and the active agent are fused or coupled post-translationally, e.g., using click chemistry; or
   (ii) the peptide and the active agent are fused or couple via chemically induced dimerization.
633. The peptide of any one of embodiments 615-632, wherein the peptide is present N-terminal relative to the active agent.
634. The peptide of any one of embodiments 615-632, wherein the peptide is present C-terminal relative to the active agent.
635. The peptide of any one of embodiment 615-620, 625, or 627-634, wherein the peptide is present or coupled to a carrier, e.g., an exosome, a microvesicle, or a lipid nanoparticle (LNP), optionally, wherein the carrier comprises a therapeutic agent (e.g., an RNAi agent (e.g., an dsRNA, a siRNA, a shRNA, a pre-miRNA, a pri-miRNA, a miRNA, a stRNA, a lncRNA, a piRNA, an antisense oligonucleotide agent (ASO), or a snoRNA), an mRNA, a ribonucleoprotein complex (e.g., a Cas9/gRNA complex), or a circRNA).
636. The peptide of embodiment 635, wherein the peptide is present on the surface of the carrier, optionally wherein at least 10%, 20%, 30%, 40%, 50%, 60%, 70%, or 80% of the surface of the carrier comprises at least 1-5, e.g., at least 1, 2, 3, 4, or 5 peptides according to any one of embodiments 422-436 or 609-614.
637. The AAV capsid variant of any one of embodiments 1-420, wherein the AAV capsid variant comprises an amino acid sequence comprising at least 5, 6, 7, 8, or 9 consecutive amino acids from the amino acid sequence of PLNGAVHLY (SEQ ID NO: 3648), wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); or
   (v) the 9 consecutive amino acids comprise PLNGAVHLY (SEQ ID NO: 3648),
   wherein the AAV capsid variant comprises: (a) a VP1 protein comprising the amino acid sequence of SEQ ID NO: 138 or SEQ ID NO: 5, 8, or 3636; (b) a VP2 protein comprising the amino acid sequence of positions 138-736 of SEQ ID NO: 138 or positions 138-743 of SEQ ID NO: 5, 8, or 3636; (c) a VP3 protein comprising the amino acid sequence of positions 203-736 of SEQ ID NO: 138 or positions 203-743 of SEQ ID NO: 5, 8, or 3636; or (d) an amino acid sequence with at least 90% (e.g., at least about 95, 96, 97, 98, or 99%) sequence identity to any of the amino acid sequences in (a)-(c).
638. The AAV capsid variant of any one of embodiments 1-420 or 637, wherein the AAV capsid variant comprises an amino acid sequence comprising at least 5, 6, 7, 8, or 9 consecutive amino acids from the amino acid sequence of PLNGAVHLY (SEQ ID NO: 3648), wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); or
   (v) the 9 consecutive amino acids comprise PLNGAVHLY (SEQ ID NO: 3648),
   wherein the AAV capsid variant comprises the amino acid sequence of any one of SEQ ID NOs: 5, 8, or 3636 or an amino acid sequence with at least 90% (e.g., at least about 95, 96, 97, 98, or 99%) sequence identity to any one of SEQ ID NOs: 5, 8, or 3636.
639. The AAV capsid variant of any one of embodiments 1-420, 637, or 638, wherein the AAV capsid variant comprises one or two, but no more than three different amino acids (e.g., substitutions, e.g., conservative substitutions) relative to the amino acid sequence of PLNGAVHLY (SEQ ID NO: 3648), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
640. The AAV capsid variant of any one of embodiments 1-420 or 637-639, wherein the AAV capsid variant comprises one or two, but no more than three different amino acids (e.g., substitutions, e.g., conservative substitutions) relative to the amino acid sequence of PLNGAVHLY (SEQ ID NO: 3648), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
641. The AAV capsid variant of any one of embodiments 637-640, wherein the amino acid sequence is present immediately subsequent to position 586, numbered according to any one of SEQ ID NO: 5, 8, 138, or 3636, optionally wherein the amino acid replaces positions 587 and 588 (e.g., A587 and Q588), numbered according to SEQ ID NO: 138.
642. The AAV capsid variant of any one of embodiments 637-641, wherein the amino acid sequence corresponds to positions 587-595 of SEQ ID NO: 5, 8, or 3636.
643. The AAV capsid variant of any one of embodiments 1-420, wherein the AAV capsid variant comprises one or two, but no more than three different amino acids (e.g., substitutions, e.g. conservative substitutions) relative to the amino acid sequence of PLNGAVHLYAQAQLSPVKN (SEQ ID NO: 566), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
644. The AAV capsid variant of any one of embodiments 1-420 or 643, wherein the AAV capsid variant comprises one or two, but no more than three different amino acids (e.g., substitutions, e.g., conservative substitutions) relative to the amino acid sequence of PLNGAVHLYAQAQLSPVKN (SEQ ID NO: 566), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
645. The AAV capsid variant of any one of embodiments 1-420, wherein the AAV capsid variant comprises one or two, but no more than three different amino acids (e.g., substitutions, e.g., conservative substitutions) relative to the amino acid sequence of PLNGAVHLYAQAQTGWVPN (SEQ ID NO: 314), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
646. The AAV capsid variant of any one of embodiments 1-420 or 645, wherein the AAV capsid variant comprises one or two, but no more than three different amino acids (e.g., substitutions, e.g., conservative substitutions) relative to the amino acid sequence of PLNGAVHLYAQAQTGWVPN (SEQ ID NO: 314), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
647. The AAV capsid variant of any one of embodiments 643-646, wherein the amino acid sequence is present immediately subsequent to position 586, numbered according to any one of SEQ ID NO: 5, 8, 138, or 3636, optionally wherein the amino acid replaces positions 587-598, numbered according to SEQ ID NO: 138.
648. The AAV capsid variant of any one of embodiments 643-647, wherein the amino acid sequence corresponds to positions 587-605 of SEQ ID NO: 5 or 8.
649. The AAV capsid variant of any one of embodiments 643, 644, 647, or 648, which comprises the amino acid sequence of SEQ ID NO: 8.
650. The AAV capsid variant of any one of embodiments 645-648, which comprises the amino acid sequence of SEQ ID NO: 5.
651. An AAV particle comprising the AAV capsid variant of any one of embodiments 637-650, optionally wherein the AAV particle is according to any one of embodiments 429-472.
652. A method of treating a subject having or diagnosed with having a genetic disorder (e.g., a monogenic disorder), a polygenic disorder, a neurological disorder (e.g., a neurodegenerative disorder), a muscular disorder, a neuromuscular disorder, or a neuro-oncological disorder, comprising administering to the subject an effective amount of an AAV particle comprising the AAV capsid variant of any one of embodiments 637-650 or the AAV particle of embodiment 651.
653. A method of delivering a payload to a cell or tissue (e.g., a CNS cell or a CNS tissue), comprising administering an effective amount of the an AAV particle comprising the AAV capsid variant of any one of embodiments 637-650 or the AAV particle of embodiment 651, optionally wherein the cell or tissue is within a subject and further optionally wherein the subject has, has been diagnosed with having, or is at risk of having a genetic disorder (e.g., a monogenic disorder), a polygenic disorder, a neurological disorder (e.g., a neurodegenerative disorder), a muscular disorder, a neuromuscular disorder, or a neuro-oncological disorder.

### ADDITIONAL ENUMERATED EMBODIMENTS

1. An AAV capsid polypeptide, e.g., an AAV capsid variant, comprising:
(a) the amino acid sequence of any of SEQ ID NO: 1725-3622 or 3648-3659;
(b) at least 3, 4, 5, 6, 7, 8, or 9 consecutive amino acids from the amino acid sequence of any of SEQ ID NO: 1725-3622 or 3648-3659; or
(c) an amino acid sequence comprising no more than four modifications, e.g., substitutions, relative to the amino acid sequence of any of SEQ ID NO: 1725-3622 or 3648-3659, and
optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
2. An AAV capsid polypeptide, e.g., an AAV capsid variant, comprising:
(a) the amino acid sequence of any of SEQ ID NO: 3648-3659;
(b) at least 3, 4, 5, 6, 7, 8, or 9 consecutive amino acids from the amino acid sequence of any of SEQ ID NO: 3648-3659; or
(c) an amino acid sequence comprising no more than four modifications, e.g., substitutions, relative to the amino acid sequence of any of SEQ ID NO: 3648-3659, and
optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
3. The AAV capsid polypeptide, e.g., the AAV capsid variant, of embodiment 1 or 2, which comprises at least 3, 4, 5, 6, 7, 8, or 9 consecutive amino acids from the amino acid sequence of any of SEQ ID NO: 3648-3659.
4. The AAV capsid polypeptide, e.g., the AAV capsid variant, of any one of embodiments 1-3, wherein the 3 consecutive amino acids comprise PLN.
5. The AAV capsid polypeptide, e.g., the AAV capsid variant, of any one of embodiments 1-4, wherein the 4 consecutive amino acids comprise PLNG (SEQ ID NO: 3678).
6. The AAV capsid polypeptide, e.g., the AAV capsid variant, of any one of the preceding embodiments, wherein the 5 consecutive amino acids comprise PLNGA (SEQ ID NO: 3679).
7. The AAV capsid polypeptide, e.g., the AAV capsid variant, of any one of the preceding embodiments, wherein the 6 consecutive amino acids comprise PLNGAV (SEQ ID NO: 3680).
8. The AAV capsid polypeptide, e.g., the AAV capsid variant, of any one of the preceding embodiments, wherein the 7 consecutive amino acids comprise PLNGAVH (SEQ ID NO: 3681).
9. The AAV capsid polypeptide, e.g., the AAV capsid variant, of any one of the preceding embodiments, wherein the 8 consecutive amino acids comprise PLNGAVHL (SEQ ID NO: 3682).
10. The AAV capsid polypeptide, e.g., the AAV capsid variant, of any one of the preceding embodiments, wherein the 9 consecutive amino acids comprise PLNGAVHLY (SEQ ID NO: 3648).
11. The AAV capsid polypeptide, e.g., the AAV capsid variant, of any one of embodiments 1-3, wherein the 3 consecutive amino acids comprise YST.
12. The AAV capsid polypeptide, e.g., the AAV capsid variant, of any one of embodiments 1-3 or 11, wherein the 4 consecutive amino acids comprise YSTD (SEQ ID NO: 3690).
13. The AAV capsid polypeptide, e.g., the AAV capsid variant, of any one of embodiments 1-3 or 11-12, wherein the 5 consecutive amino acids comprise YSTDE (SEQ ID NO: 3691) or YSTDV (SEQ ID NO: 3700).
14. The AAV capsid polypeptide, e.g., the AAV capsid variant, of any one of embodiments 1-3 or 11-13, wherein the 6 consecutive amino acids comprise YSTDER (SEQ ID NO: 3692) or YSTDVR (SEQ ID NO: 3701).
15. The AAV capsid polypeptide, e.g., the AAV capsid variant, of any one of embodiments 1-3, wherein the 3 consecutive amino acids comprise IVM.
16. The AAV capsid polypeptide, e.g., the AAV capsid variant, of any one of embodiments 1-3 or 15, wherein the 4 consecutive amino acids comprise IVMN (SEQ ID NO: 3693).
17. The AAV capsid polypeptide, e.g., the AAV capsid variant, of any one of embodiments 1-3 or 15-16, wherein the 5 consecutive amino acids comprise IVMNS (SEQ ID NO: 3694).
18. The AAV capsid polypeptide, e.g., the AAV capsid variant, of any one of embodiments 1-3 or 15-17, wherein the 6 consecutive amino acids comprise IVMNSL (SEQ ID NO: 3695).
19. The AAV capsid polypeptide, e.g., the AAV capsid variant, of any one of embodiments 1-3 or 15-18, wherein the 7 consecutive amino acids comprise IVMNSLK (SEQ ID NO: 3651).
20. The AAV capsid polypeptide, e.g., the AAV capsid variant of any one of embodiments 1-19, which comprises an amino acid sequence comprising at least one, two, or three but no more than four modifications, e.g., substitutions, relative to the amino acid sequence of any of SEQ ID NO: 3648-3659.
21. The AAV capsid polypeptide, e.g., the AAV capsid variant, of any one of embodiments 1-10 or 20, comprising the amino acid sequence of PLNGAVHLY (SEQ ID NO: 3648), or an amino acid sequence having at least one, two, or three but no more than four modifications, e.g., substitutions, relative to the amino acid sequence of PLNGAVHLY (SEQ ID NO: 3648), optionally, wherein position 7 is H.
22. The AAV capsid polypeptide, e.g., the AAV capsid variant, of any one of embodiments 1-3 or 20, comprising the amino acid sequence of RDSPKGW (SEQ ID NO: 3649), or an amino acid sequence having at least one, two, or three modifications but no more than four modifications, e.g., substitutions, relative to the amino acid sequence of RDSPKGW (SEQ ID NO: 3649).
23. The AAV capsid polypeptide, e.g., the AAV capsid variant, of any one of embodiments 1-3 or 15-20, comprising the amino acid sequence of IVMNSLK (SEQ ID NO: 3651), or an amino acid sequence having at least one, two, or three modifications but no more than four modifications, e.g., substitutions, relative to the amino acid sequence of IVMNSLK (SEQ ID NO: 3651).
24. The AAV capsid polypeptide, e.g., the AAV capsid variant, of any one of embodiments 1-3, 11-14, or 20, comprising the amino acid sequence of YSTDVRM (SEQ ID NO: 3650), or an amino acid sequence having at least one, two, or three modifications but no more than four modifications, e.g., substitutions, relative to the amino acid sequence of YSTDVRM (SEQ ID NO: 3650).
25. The AAV capsid polypeptide, e.g., the AAV capsid variant, of any one of embodiments 1-3 or 20, comprising the amino acid sequence of RESPRGL (SEQ ID NO: 3652), or a sequence having at least one, two, or three modifications but no more than four modifications, e.g., substitutions, relative to the amino acid sequence of RESPRGL (SEQ ID NO: 3652).
26. The AAV capsid polypeptide, e.g., the AAV capsid variant, of any one of the preceding embodiments, which comprises the amino acid sequence of any of SEQ ID NO: 3648-3659.
27. The AAV capsid polypeptide, e.g., the AAV capsid variant, of any one of embodiments 1-26, comprising:
(i) an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto; or
(ii) an amino acid sequence encoded by a nucleotide sequence comprising at least one, two, three, four, five, six, or seven modifications but no more than ten modifications of the nucleotide sequence of any of SEQ ID NOs: 3660-3671.
28. The AAV capsid polypeptide, e.g., the AAV capsid variant, of any one of embodiments 1-27, wherein the nucleotide sequence encoding the AAV capsid variant comprises:
(i) the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of any of SEQ ID NOs: 3660-3671.
29. The AAV capsid polypeptide, e.g., the AAV capsid variant, of any one of embodiments 1-10, 20-21, or 26-28, comprising an amino acid sequence encoded by:
(i) the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3660.
30. The AAV capsid polypeptide, e.g., the AAV capsid variant, of any one of embodiments 1-10, 20-21, or 26-29, wherein the nucleotide sequence encoding the AAV capsid variant comprises:
(i) the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3660.
31. The AAV capsid polypeptide, e.g., the AAV capsid variant, of any one of embodiments 1-3, 20, 22, or 26-28, comprising an amino acid sequence encoded by:
(i) the nucleotide sequence of SEQ ID NO: 3661, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3661.
32. The AAV capsid polypeptide, e.g., the AAV capsid variant, of any one of embodiments 1-3, 20, 22, 26-28, or 31, wherein the nucleotide sequence encoding the AAV capsid variant comprises:
(i) the nucleotide sequence of SEQ ID NO: 3661, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3661.
33. The AAV capsid polypeptide, e.g., the AAV capsid variant, of any one of embodiments 1-3, 11-14, 20, 24, or 26-28, comprising an amino acid sequence encoded by:
(i) the nucleotide sequence of SEQ ID NO: 3662, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3662.
34. The AAV capsid polypeptide, e.g., the AAV capsid variant, of any one of embodiments 1-3, 11-14, 20, 24, 26-28, or 33, wherein the nucleotide sequence encoding the AAV capsid variant comprises:
(i) the nucleotide sequence of SEQ ID NO: 3662, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3662.
35. The AAV capsid polypeptide, e.g., the AAV capsid variant, of any one of embodiments 1-3, 15-20, 23, or 26-28, comprising an amino acid sequence encoded by:
(i) the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3663.
36. The AAV capsid polypeptide, e.g., the AAV capsid variant, of any one of embodiments 1-3, 15-20, 23, 26-28, or 35, wherein the nucleotide sequence encoding the AAV capsid variant comprises:
(i) the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3663.
37. The AAV capsid polypeptide, e.g., the AAV capsid variant, of any one of embodiments 1-3, 20, or 25-28, comprising an amino acid sequence encoded by:
(i) the nucleotide sequence of SEQ ID NO: 3664, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3664.
38. The AAV capsid polypeptide, e.g., the AAV capsid variant, of any one of embodiments 1-3, 20, 25-28, or 37, wherein the nucleotide encoding the AAV capsid variant comprises:
(i) the nucleotide sequence of SEQ ID NO: 3664, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3664.
39. The AAV capsid polypeptide, e.g., the AAV capsid variant, of any one of the preceding embodiments, wherein the amino acid sequence is present in loop VIII.
40. The AAV capsid polypeptide, e.g., the AAV capsid variant, of any one of embodiments 1-10, 20-21, 26-30, or 39, wherein the amino acid sequence is present immediately subsequent to position 586, relative to a reference sequence numbered according to the amino acid sequence of SEQ ID NO: 138.
41. The AAV capsid polypeptide, e.g., the AAV capsid variant, of any one of embodiments 1-3, 15-20, 23, 26-28, 35-36, or 39, wherein the amino acid sequence is present immediately subsequent to position 588, relative to a reference sequence numbered according to the amino acid sequence of SEQ ID NO: 138.
42. The AAV capsid polypeptide, e.g., the AAV capsid variant, of any one of embodiments 1-3, 11-14, 20, 22, 24-28, 31-34, 37-38, or 39 wherein the amino acid sequence is present immediately subsequent to position 589, relative to a reference sequence numbered according to the amino acid sequence of SEQ ID NO: 138.
43. The AAV capsid polypeptide, e.g., the AAV capsid variant of any one of embodiments 1-42, which comprises an amino acid residue other than “A” at position 587 and/or an amino acid residue other than “Q” at position 588, numbered according to SEQ ID NO: 138.
44. The AAV capsid polypeptide, e.g., the AAV capsid variant, of any one of embodiments 1-10, 20-21, 26-30, 39-40, or 43, comprising the amino acid sequence of PLNGAVHLY (SEQ ID NO: 3648), wherein the amino acid sequence of PLNGAVHLY (SEQ ID NO: 3648) is present immediately subsequent to position 586, relative to a reference sequence numbered according to the amino acid sequence of SEQ ID NO: 138.
45. The AAV capsid polypeptide, e.g., the AAV capsid variant, of any one of embodiments 1-3, 20, 26-28, 39-40, or 43, comprising the amino acid sequence of GGTLAVVSL (SEQ ID NO: 3654), wherein the amino acid sequence of GGTLAVVSL (SEQ ID NO: 3654) is present immediately subsequent to position 586, relative to a reference sequence numbered according to the amino acid sequence of SEQ ID NO: 138.
46. The AAV capsid polypeptide, e.g., the AAV capsid variant, of any one of embodiments 1-3, 15-20, 23, 26-28, 35-36, 39, or 41, comprising the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138.
47. The AAV capsid polypeptide, e.g., the AAV capsid variant, of any one of embodiments 1-3, 11-14, 20, 22, 24-28, 31-34, 37-38, 39, or 42, comprising the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.
48. The AAV capsid polypeptide, e.g., the AAV capsid variant, of any one of the preceding embodiments, which further comprises the amino acid substitution of K449R, numbered according to SEQ ID NO: 138.
49. The AAV capsid polypeptide, e.g., the AAV capsid variant, of any one of the preceding embodiments, which further comprises a modification, e.g., an insertion, substitution, and/or deletion, in loop I, II, IV and/or VI.
50. The AAV capsid polypeptide, e.g., the AAV capsid variant, of any one of the preceding embodiments, which comprises an amino acid sequence having at least one, two or three modifications but not more than 30, 20 or 10 modifications of the amino acid sequence of SEQ ID NO: 138.
51. The AAV capsid polypeptide, e.g., the AAV capsid variant, of any one of the preceding embodiments, which comprises the amino acid sequence of SEQ ID NO: 138, or an amino acid sequence with at least 80% (e.g., at least about 85, 90, 95, 96, 97, 98, or 99%) sequence identity thereto.
52. The AAV capsid polypeptide, e.g., the AAV capsid variant, of any one of the preceding embodiments, which comprises the amino acid sequence of SEQ ID NO: 138.
53. The AAV capsid polypeptide, e.g., the AAV capsid variant, of any one of the preceding embodiments, which comprises an amino acid sequence encoded by the nucleotide sequence of SEQ ID NO: 137, or a sequence with at least 80% (e.g., at least about 85, 90, 95, 96, 97, 98, or 99%) sequence identity thereto.
54. The AAV capsid polypeptide, e.g., the AAV capsid variant, of any one of the preceding embodiments, wherein the nucleotide sequence encoding the capsid variant comprises the nucleotide sequence of SEQ ID NO: 137, or a sequence with at least 80% (e.g., at least about 85, 90, 95, 96, 97, 98, or 99%) sequence identity thereto.
55. The AAV capsid polypeptide, e.g., the AAV capsid variant, of any one of the preceding embodiments, which comprises a VP1 protein, a VP2 protein, a VP3 protein, or a combination thereof.
56. The AAV capsid polypeptide, e.g., the AAV capsid variant, of any one of the preceding embodiments, which comprises the amino acid sequence corresponding to positions 138-743, e.g., a VP2, of any one of SEQ ID NOs: 3636-3647, or a sequence with at least 80% (e.g., at least about 85, 90, 95, 96, 97, 98, or 99%) sequence identity thereto.
57. The AAV capsid polypeptide, e.g., the AAV capsid variant, of any one of the preceding embodiments, which comprises the amino acid sequence corresponding to positions 203-743, e.g., a VP3, of any one of SEQ ID NOs: 3636-3647, or a sequence with at least 80% (e.g., at least about 85, 90, 95, 96, 97, 98, or 99%) sequence identity thereto.
58. The AAV capsid polypeptide, e.g., the AAV capsid variant, of any one of the preceding embodiments, which comprises the amino acid sequence of any one of SEQ ID NOs: 3636-3647, or an amino acid sequence with at least 80% (e.g., at least about 85, 90, 95, 96, 97, 98, or 99%) sequence identity thereto.
59. The AAV capsid polypeptide, e.g., the AAV capsid variant, of any one of the preceding embodiments, which comprises an amino acid sequence comprising at least one, two or three modifications, but not more than 30, 20 or 10 modifications of the amino acid sequence relative to any one of SEQ ID NOs: 3636-3647.
60. The AAV capsid polypeptide, e.g., the AAV capsid variant, of any one of the preceding embodiments, which comprises an amino acid sequence encoded by the nucleotide sequence of any one of SEQ ID NOs: 3623-3635, or a nucleotide sequence with at least 80% (e.g., at least about 85, 90, 95, 96, 97, 98, or 99%) sequence identity thereto.
61. The AAV capsid polypeptide, e.g., the AAV capsid variant, of any one of the preceding embodiments, wherein the nucleotide sequence encoding the capsid variant comprises the nucleotide sequence of any one of SEQ ID NOs: 3623-3635, or a nucleotide sequence with at least 80% (e.g., at least about 85, 90, 95, 96, 97, 98, or 99%) sequence identity thereto.
62. The AAV capsid polypeptide, e.g., the AAV capsid variant, of any one of embodiments 1-10, 20-21, 26-30, 39-40, 43-44, or 48-61, wherein the nucleotide sequence encoding the capsid variant comprises the nucleotide sequence of SEQ ID NO: 3623, or a nucleotide sequence with at least 80% (e.g., at least about 85, 90, 95, 96, 97, 98, or 99%) sequence identity thereto.
63. The AAV capsid polypeptide, e.g., the AAV capsid variant, of any one of embodiments 1-3, 15-20, 23, 26-28, 35-36, 39, 41, 46, or 48-61, wherein the nucleotide sequence encoding the capsid variant comprises the nucleotide sequence of SEQ ID NO: 3627, or a nucleotide sequence with at least 80% (e.g., at least about 85, 90, 95, 96, 97, 98, or 99%) sequence identity thereto.
64. The AAV capsid polypeptide, e.g., the AAV capsid variant, of any one of the preceding embodiments, wherein the nucleotide sequence encoding the capsid variant is codon optimized.
65. An AAV capsid polypeptide, e.g., an AAV capsid variant, comprising the amino acid sequence of any one of embodiments 1-10, 20-21, 26-30, 39-40, 43-44, 48-62, or 64, and further comprising an amino acid sequence at least 95% identical to SEQ ID NO: 3636.
66. An AAV capsid polypeptide, e.g., an AAV capsid variant, comprising the amino acid sequence of SEQ ID NO: 3636, and optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
67. An AAV capsid polypeptide, e.g., an AAV capsid variant, comprising the amino acid sequence of any one of embodiments 1-3, 20, 22, 26-28, 31-32, 39, 42, 47-61, or 64, and further comprising an amino acid sequence at least 95% identical to SEQ ID NO: 3637.
68. An AAV capsid polypeptide, e.g., an AAV capsid variant, comprising the amino acid sequence of SEQ ID NO: 3637.
69. An AAV capsid polypeptide, e.g., an AAV capsid variant, comprising the amino acid sequence of any one of embodiments 1-3, 11-12, 20, 24, 26-28, 33-34, 39, 42, 47-61, or 64, and further comprising an amino acid sequence at least 95% identical to SEQ ID NO: 3638.
70. An AAV capsid polypeptide, e.g., an AAV capsid variant, comprising the amino acid sequence of SEQ ID NO: 3638.
71. An AAV capsid polypeptide, e.g., an AAV capsid variant, comprising the amino acid sequence of any one of embodiments 1-3, 15-20, 23, 26-28, 35-36, 39, 41, 46, 48-61, or 63-64, and further comprising an amino acid sequence at least 95% identical to SEQ ID NO: 3639.
72. An AAV capsid polypeptide, e.g., an AAV capsid variant, comprising the amino acid sequence of SEQ ID NO: 3639.
73. An AAV capsid polypeptide, e.g., an AAV capsid variant, comprising the amino acid sequence of any one of embodiments 1-3, 20, 25-28, 37-39, 42, 47-61, or 64, and further comprising an amino acid sequence at least 95% identical to SEQ ID NO: 3640.
74. An AAV capsid polypeptide, e.g., an AAV capsid variant, comprising the amino acid sequence of SEQ ID NO: 3640.
75. An AAV capsid polypeptide, e.g., an AAV capsid variant, comprising the amino acid sequence of SEQ ID NO: 3641.
76. An AAV capsid polypeptide, e.g., an AAV capsid variant, comprising the amino acid sequence of SEQ ID NO: 3642.
77. An AAV capsid polypeptide, e.g., an AAV capsid variant, comprising the amino acid sequence of SEQ ID NO: 3643.
78. An AAV capsid polypeptide, e.g., an AAV capsid variant, comprising the amino acid sequence of SEQ ID NO: 3644.
79. An AAV capsid polypeptide, e.g., an AAV capsid variant, comprising the amino acid sequence of SEQ ID NO: 3645.
80. An AAV capsid polypeptide, e.g., an AAV capsid variant, comprising the amino acid sequence of SEQ ID NO: 3646.
81. An AAV capsid polypeptide, e.g., an AAV capsid variant, comprising the amino acid sequence of SEQ ID NO: 3647.
82. An AAV capsid polypeptide, e.g., an AAV capsid variant, comprising an amino acid sequence encoded by the nucleotide sequence of any one of SEQ ID NOs: 3623-3635, or a nucleotide sequence at least 95% identical thereto, and optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
83. The AAV capsid polypeptide, e.g., the AAV capsid variant, of any one of the preceding embodiments, which does not comprise an insert sequence present immediately subsequent to position 586, 588, or 589 numbered relative to SEQ ID NO: 138, having at least 5 consecutive amino acids corresponding to positions 586 to 594 numbered relative to SEQ ID NO: 138, of any of SEQ ID NOs: 1-1724, e.g., as described in Table 1 of WO2020223276.
84. The AAV capsid polypeptide, e.g., the AAV capsid variant, of any one of the preceding embodiments, which does not comprise the amino acid sequence of TLAVPFK (SEQ ID NO: 1262) present immediately subsequent to position 588, numbered according to SEQ ID NO: 138.
85. The AAV capsid polypeptide, e.g., the AAV capsid variant, of any one of the preceding embodiments, which has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.
86. The AAV capsid polypeptide, e.g., the AAV capsid variant, of any one of embodiments 1-12, 15-44, 46-74, 82-85, which transduces a brain region, e.g., selected from dentate nucleus, cerebellar cortex, cerebral cortex, brain stem, hippocampus, thalamus and putamen, optionally wherein the level of transduction is at least 5, 10, 50, 100, 200, 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 5.
87. The AAV capsid polypeptide, e.g., the AAV capsid variant, of any one of the preceding embodiments, which is enriched at least about 5, 6, 7, 8, 9, or 10-fold, in the brain compared to a reference sequence of SEQ ID NO: 138, e.g., when measured by an assay as described in Example 4.
88. The AAV capsid polypeptide, e.g., the AAV capsid variant, of any one of embodiments 1-14, 20-22, 24-34, 39-40, 42-44, 47-62, 64-70, 79-80, 82-87, which is enriched at least about 20, 30, 40, or 50-fold in the brain compared to a reference sequence of SEQ ID NO: 138, e.g., when measured by an assay as described in Example 4.
89. The AAV capsid polypeptide, e.g., the AAV capsid variant, of any one of embodiments 1-10, 20-22, 26-32, 39-40, 42-44, 47-62, 64-68, 82-88, which is enriched at least about 100, 200, 300, or 400-fold in the brain compared to a reference sequence of SEQ ID NO: 138, e.g., when measured by an assay as described in Example 4.
90. The AAV capsid polypeptide, e.g., the AAV capsid variant, of any one of embodiments 1-10, 20-21, 26-30, 39-40, 43-44, 48-62, 64-66, 82-89, which delivers an increased level of viral genomes to a brain region, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 5).
91. The AAV capsid polypeptide, e.g., the AAV capsid variant, of any one of embodiments 1-12, 15-44, 46-74, 82-90, which delivers an increased level of a payload to a brain region, optionally wherein the level of the payload is increased by at least 5, 10, 50, 100, 200, 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 5).
92. The AAV capsid polypeptide, e.g., the AAV capsid variant, of embodiment 91, wherein the brain region comprises a frontal cortex, sensory cortex, motor cortex, putamen, thalamus, cerebellar cortex, dentate nucleus, caudate, and/or hippocampus.
93. The AAV capsid polypeptide, e.g., the AAV capsid variant, of any one of embodiments 1-12, 15-44, 46-74, 82-92, which delivers an increased level of a payload to a spinal cord region, optionally wherein the level of the payload is increased by at least 10, 20, 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 5).
94. The AAV capsid polypeptide, e.g., the AAV capsid variant, of embodiment 93, wherein the spinal cord region comprises a cervical, thoracic, and/or lumbar region.
95. The AAV capsid polypeptide, e.g., the AAV capsid variant, of any one of embodiments 1-3, 15-20, 23, 26-28, 35-36, 39, 41, 46, 48-61, 63-64, 71-72, 82-87, or 91-94, which shows preferential transduction in a brain region relative to the transduction in the dorsal root ganglia (DRG).
96. The AAV capsid polypeptide, e.g., the AAV capsid variant, of any one of embodiments 1-10, 20-21, 26-30, 39-40, 43-44, 48-62, 64-66, 82-94, wherein the capsid variant:
(i) is enriched at least about 300, or 400-fold in the brain compared to a reference sequence of SEQ ID NO: 138, e.g., when measured by an assay as described in Example 4;
(ii) transduces a brain region, e.g., selected from dentate nucleus, cerebellar cortex, cerebral cortex, brain stem, hippocampus, thalamus and putamen, wherein the level of transduction is at least 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 5;
(iii) delivers an increased level of a payload to a brain region, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 5), optionally wherein the brain region comprises a frontal cortex, sensory cortex, motor cortex, putamen, thalamus, cerebellar cortex, dentate nucleus, caudate, and/or hippocampus;
(iv) delivers an increased level of a payload to a spinal cord region, optionally wherein the level of the payload is increased by at least 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 5), optionally wherein the spinal cord region comprises a cervical, thoracic, and/or lumbar region; and/or
(v) delivers an increased level of viral genomes to a brain region, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 5), optionally wherein the brain region comprises a frontal cortex, sensory cortex, motor cortex, putamen, thalamus, cerebellar cortex, dentate nucleus, caudate, and/or hippocampus.
97. An AAV capsid polypeptide, e.g., an AAV capsid variant comprising: (a) the amino acid sequence of PLNGAVHLY (SEQ ID NO: 3648); (b) an amino acid sequence comprising at least one, two, or three modifications but no more than four modifications, e.g., substitutions, relative to the amino acid sequence of PLNGAVHLY (SEQ ID NO: 3648); or (c) at least 3, 4, 5, 6, 7, 8, or 9 consecutive amino acids from the amino acid sequence of PLNGAVHLY (SEQ ID NO: 3648); and wherein the capsid variant:
(i) is enriched at least about 300 or 400-fold in the brain compared to a reference sequence of SEQ ID NO: 138, e.g., when measured by an assay as described in Example 4;
(ii) transduces a brain region, e.g., selected from dentate nucleus, cerebellar cortex, cerebral cortex, brain stem, hippocampus, thalamus and putamen, wherein the level of transduction is at least 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 5;
(iii) delivers an increased level of a payload to a brain region, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 5), optionally wherein the brain region comprises a frontal cortex, sensory cortex, motor cortex, putamen, thalamus, cerebellar cortex, dentate nucleus, caudate, and/or hippocampus;
(iv) delivers an increased level of a payload to a spinal cord region, optionally wherein the level of the payload is increased by at least 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 5), optionally wherein the spinal cord region comprises a cervical, thoracic, and/or lumbar region; and/or
(v) delivers an increased level of viral genomes to a brain region, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 5), optionally wherein the brain region comprises a frontal cortex, sensory cortex, motor cortex, putamen, thalamus, cerebellar cortex, dentate nucleus, caudate, and/or hippocampus; and
optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
98. The AAV capsid polypeptide, e.g., the AAV capsid variant, of any one of embodiments 1-3, 15-20, 23, 26-28, 35-36, 39, 41, 46, 48-61, 63-64, 71-72, 82-87, or 91-95, wherein the AAV capsid variant has an increased tropism for a muscle cell or tissue, e.g., a heart tissue, relative to the tropism of a reference sequence comprising the amino acid sequence of SEQ ID NO: 138.
99. The AAV capsid polypeptide, e.g., the AAV capsid variant, of any one of embodiments 1-3, 15-20, 23, 26-28, 35-36, 39, 41, 46, 48-61, 63-64, 71-72, 82-87, 92-95, or 98, which delivers an increased level of a payload to a muscle region, optionally wherein the level of the payload is increased by at least 10, 15, 20, 30, or 40-fold, as compared to a reference sequence of SEQ ID NO: 138, e.g., when measured by an assay, e.g., an IHC assay or a RT-ddPCR assay (e.g., as described in Example 5).
100. The AAV capsid polypeptide, e.g., the AAV capsid variant, of embodiment 98 or 99, wherein the muscle region comprises a heart muscle, quadriceps muscle, and/or a diaphragm muscle region.
101. The AAV capsid polypeptide, e.g., the AAV capsid variant, of any one of embodiments 98-100, wherein the muscle region comprises a heart muscle region, e.g., a heart atrium muscle region or a heart ventricle muscle region.
102. The AAV capsid polypeptide, e.g., the AAV capsid variant, of any one of the preceding embodiments, which is isolated, e.g., recombinant.
103. A polynucleotide encoding the polypeptide, e.g., the AAV capsid variant of any one of embodiments 1-102.
104. The polynucleotide of embodiment 103, which comprises:
(i) a nucleotide sequence comprising at least one, two, three, four, five, six, or seven modifications but no more than ten modifications of the nucleotide sequences of any of SEQ ID NOs: 3660-3671; or
(ii) the nucleotide sequence of any one of SEQ ID NOs: 3660-3671, or nucleotide sequence substantially identical (e.g., having at least 70%, 75%, 80%, 85%, 90%, 92%, 95%, 97%, 98%, or 99% sequence identity) thereto.
105. The polynucleotide of embodiment 103 or 104, which comprises the nucleotide sequence of any one of SEQ ID NOs: 3623-3635, or a nucleotide sequence with at least 80% (e.g., at least about 85, 90, 95, 96, 97, 98, or 99%) sequence identity thereto.
106. The polynucleotide of any one of embodiments 125-127, which comprises a nucleotide sequence that is codon optimized.
107. A peptide comprising:
(a) the amino acid sequence of any of SEQ ID NO: 1725-3622 or 3648-3659;
(b) an amino acid sequence comprising no more than four modifications, e.g., substitutions, relative to the amino acid sequence of any of SEQ ID NO: 1725-3622 or 3648-3659; or
(c) at least 3, 4, 5, 6, 7, 8, or 9 consecutive amino acids from the amino acid sequence of any of SEQ ID NO: 1725-3622 or 3648-3659.
108. An AAV capsid polypeptide, e.g., an AAV capsid variant, comprising the peptide of embodiment 107.
109. A polynucleotide encoding the peptide of embodiment 107.
110. A polynucleotide encoding an AAV capsid polypeptide, e.g., an AAV capsid variant comprising:
(a) the amino acid sequence of any of SEQ ID NO: 1725-3622 or 3648-3659;
(b) an amino acid sequence comprising no more than four modifications, e.g., substitutions, relative to the amino acid sequence of any of SEQ ID NO: 1725-3622 or 3648-3659; or
(c) at least 3, 4, 5, 6, 7, 8, or 9 consecutive amino acids from the amino acid sequence of any of SEQ ID NO: 1725-3622 or 3648-3659;
optionally wherein:
(i) the amino acid sequence of (a), (b), and/or (c) is present immediately subsequent to position 586, 588, or 589, relative to a reference sequence numbered according to the amino acid sequence of SEQ ID NO: 138; and/or
(ii) the encoded AAV capsid polypeptide, e.g., an AAV capsid variant, further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
111. A polynucleotide encoding an AAV capsid polypeptide, e.g., an AAV capsid variant, wherein the AAV capsid variant comprises:
(i) the amino acid sequence of PLNGAVHLY (SEQ ID NO: 3648);
(ii) an amino acid sequence comprising at least one, two, or three modifications but no more than four modifications, e.g., substitutions, relative to the amino acid sequence of PLNGAVHLY (SEQ ID NO: 3648); or
(iii) at least 3, 4, 5, 6, 7, 8, or 9 consecutive amino acids from the amino acid sequence of PLNGAVHLY (SEQ ID NO: 3648);
optionally wherein:
(i) the amino acid sequence of (i), (ii), and/or (iii) is present immediately subsequent to position 586, relative to a reference sequence numbered according to the amino acid sequence of SEQ ID NO: 138;
(ii) the encoded AAV capsid polypeptide, e.g., an AAV capsid variant, further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
112. The polynucleotide of embodiment 111, which comprises:
(i) the nucleotide sequence of SEQ ID NO: 3660 or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3660.
113. A polynucleotide encoding an AAV capsid polypeptide, e.g., an AAV capsid variant, wherein the AAV capsid variant comprises:
(i) the amino acid sequence of IVMNSLK (SEQ ID NO: 3651);
(ii) an amino acid sequence comprising at least one, two, or three modifications but no more than four modifications, e.g., substitutions, relative to the amino acid sequence of IVMNSLK (SEQ ID NO: 3651); or
(iii) at least 3, 4, 5, 6, 7, 8, or 9 consecutive amino acids from the amino acid sequence of IVMNSLK (SEQ ID NO: 3651);
optionally wherein the amino acid sequence of (i), (ii), and/or (iii) is present immediately subsequent to position 588, relative to a reference sequence numbered according to the amino acid sequence of SEQ ID NO: 138.
114. The polynucleotide of embodiment 113, which comprises:
(i) the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3663.
115. A polynucleotide encoding an AAV capsid polypeptide, e.g., an AAV capsid variant, wherein the AAV capsid variant comprises:
(i) the amino acid sequence of RDSPKGW (SEQ ID NO: 3649);
(ii) an amino acid sequence comprising at least one, two, or three modifications but no more than four modifications, e.g., substitutions, relative to the amino acid sequence of RDSPKGW (SEQ ID NO: 3649); or
(iii) at least 3, 4, 5, 6, 7, 8, or 9 consecutive amino acids from the amino acid sequence of RDSPKGW (SEQ ID NO: 3649);
optionally wherein the amino acid sequence of (i), (ii), and/or (iii) is present immediately subsequent to position 589, relative to a reference sequence numbered according to the amino acid sequence of SEQ ID NO: 138.
116. The polynucleotide of embodiment 115, which comprises:
(i) the nucleotide sequence of SEQ ID NO: 3661, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3661.
117. A polynucleotide encoding an AAV capsid polypeptide, e.g., an AAV capsid variant, wherein the AAV capsid variant comprises:
(i) the amino acid sequence of YSTDVRM (SEQ ID NO: 3650);
(ii) an amino acid sequence comprising at least one, two, or three modifications but no more than four modifications, e.g., substitutions, relative to the amino acid sequence of YSTDVRM (SEQ ID NO: 3650); or
(iii) at least 3, 4, 5, 6, 7, 8, or 9 consecutive amino acids from the amino acid sequence of YSTDVRM (SEQ ID NO: 3650);
optionally wherein the amino acid sequence of (i), (ii), and/or (iii) is present immediately subsequent to position 589, relative to a reference sequence numbered according to the amino acid sequence of SEQ ID NO: 138.
118. The polynucleotide of embodiment 117, which comprises:
(i) the nucleotide sequence of SEQ ID NO: 3662, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3662.
119. A polynucleotide encoding an AAV capsid polypeptide, e.g., an AAV capsid variant, wherein the AAV capsid variant comprises:
(i) the amino acid sequence of RESPRGL (SEQ ID NO: 3652);
(ii) an amino acid sequence comprising at least one, two, or three modifications but no more than four modifications, e.g., substitutions, relative to the amino acid sequence of RESPRGL (SEQ ID NO: 3652); or
(iii) at least 3, 4, 5, 6, 7, 8, or 9 consecutive amino acids from the amino acid sequence of RESPRGL (SEQ ID NO: 3652);
optionally wherein the amino acid sequence of (i), (ii), and/or (iii) is present immediately subsequent to position 589, relative to a reference sequence numbered according to the amino acid sequence of SEQ ID NO: 138.
120. The polynucleotide of embodiment 119, which comprises:
(i) the nucleotide sequence of SEQ ID NO: 3664, or a nucleotide sequence substantially identical (e.g., having at least 70%, 75%, 80%, 85%, 90%, 92%, 95%, 97%, 98%, or 99% sequence identity) thereto; or
(ii) a nucleotide sequence comprising at least one, two, three, four, five, six, or seven modifications but no more than ten modifications of the nucleotide sequence of SEQ ID NO: 3664.
121. The polynucleotide of any one of embodiments 109-120, wherein the AAV capsid variant comprises:
(i) the amino acid sequence of any one of SEQ ID NOs: 3636-3647, or an amino acid sequence with at least 80% (e.g., at least about 85, 90, 95, 96, 97, 98, or 99%) sequence identity thereto; or
(ii) an amino acid sequence having at least one, two or three modifications but not more than 30, 20 or 10 modifications of the amino acid sequence of any one of SEQ ID NOs: 3636-3647.
122. The polynucleotide of any one of embodiments 109-121, comprising the nucleotide sequence of any one of SEQ ID NOs: 3623-3635, or a nucleotide sequence with at least 80% (e.g., at least about 85, 90, 95, 96, 97, 98, or 99%) sequence identity thereto.
123. The polynucleotide, peptide, or AAV capsid polypeptide, e.g., AAV capsid variant, of any one of embodiments 103-122, which is isolated, e.g., recombinant.
124. An AAV particle comprising the AAV capsid polypeptide, e.g., the AAV capsid variant, of any one of embodiments 1-102 or 108.
125. The AAV particle of embodiment 124, which comprises a nucleotide sequence encoding a payload.
126. The AAV particle of embodiment 125, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
127. The AAV particle of embodiment 126, wherein the therapeutic protein or functional variant thereof, e.g., a recombinant protein, is associated with (e.g., aberrantly expressed in) a neurological or neurodegenerative disorder, a muscular or neuromuscular disorder, or a neuro-oncological disorder.
128. The AAV particle of embodiment 126 or 127, the therapeutic protein or functional variant thereof is chosen from apolipoprotein E (APOE) (e.g., ApoE2, ApoE3 and/or ApoE4); human survival of motor neuron (SMN) 1 or SMN2; glucocerebrosidase (GBA1); aromatic L-amino acid decarboxylase (AADC); aspartoacylase (ASPA); tripeptidyl peptidase I (CLN2); beta-galactosidase (GLB1); N-sulphoglucosamine sulphohydrolase (SGSH); N-acetyl-alpha-glucosaminidase (NAGLU); iduronate 2-sulfatase (IDS); intracellular cholesterol transporter (NPC1); gigaxonin (GAN); or a combination thereof.
129. The AAV particle of embodiment 126, wherein the antibody or antibody binding fragment binds to:
(i) a CNS related target, e.g., an antigen associated with a neurological or neurodegenerative disorder, e.g., β-amyloid, APOE, tau, SOD1, TDP-43, huntingtin (HTT), and/or synuclein;
(ii) a muscular or neuromuscular related target, e.g., an antigen associated with a muscular or neuromuscular disorder; or
(iii) a neuro-oncology related target, e.g., an antigen associated with a neuro-oncological disorder, e.g., HER2, or EGFR (e.g., EGFRvIII).
130. The AAV particle of embodiment 126, wherein the enzyme comprises a meganuclease, a zinc finger nuclease, a TALEN, a recombinase, integrase, a base editor, a Cas9, or a fragment thereof.
131. The AAV particle of embodiment 126, wherein the component of a gene editing system comprises one or more components of a CRISPR-Cas system.
132. The AAV particle of embodiment 126 or 131, wherein the one or more components of the CRISPR-Cas system comprises a Cas9, e.g., a Cas9 ortholog or a Cpf1, and a single guide RNA (sgRNA), optionally wherein:
(i) the sgRNA is located upstream (5′) of the cas9 enzyme; or
(ii) the sgRNA is located downstream (3′) of the cas9 enzyme.
133. The AAV particle of embodiment 126, wherein the RNAi agent (e.g., a dsRNA, siRNA, shRNA, pre-miRNA, pri-miRNA, miRNA, stRNA, lncRNA, piRNA, or snoRNA), modulates, e.g., inhibits, expression of, a CNS related gene, mRNA, and/or protein.
134. The AAV particle of embodiment 133, wherein the CNS related gene is chosen from SOD1, MAPT, APOE, HTT, C9ORF72, TDP-43, APP, BACE, SNCA, ATXN1, ATXN3, ATXN7, SCN1A-SCN5A, SCN8A-SCN11A, or a combination thereof.
135. The AAV particle of any one of embodiments 124-134, which comprises a viral genome comprising a promoter operably linked to the nucleic acid sequence encoding the payload.
136. The AAV particle of embodiment 135, wherein the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-$), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nP2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.
137. The AAV particle of any one of embodiments 135 or 136, wherein the viral genome further comprises a polyA signal sequence.
138. The AAV particle of any one of embodiments 135-137, wherein the viral genome further comprises an inverted terminal repeat (ITR) sequence.
139. The AAV particle of any one of embodiments 135-138, wherein the viral genome comprises an ITR sequence positioned 5′ relative to the encoded payload.
140. The AAV particle of any one of embodiments 135-139, wherein the viral genome comprises an ITR sequence positioned 3′ relative to the encoded payload.
141. The AAV particle of any one of embodiments 135-140, wherein the viral genome comprises an ITR sequence positioned 5′ relative to the encoded payload and an ITR sequence positioned 3′ relative to the encoded payload.
142. The AAV particle of any one of embodiments 135-141, wherein the viral genome further comprises an enhancer, a Kozak sequence, an intron region, and/or an exon region.
143. The AAV particle of any one of embodiments 135-142, wherein the viral genome further comprises a miR binding site, e.g., a miR binding site that modulates, e.g., reduces, expression of the payload encoded by the viral genome in a cell or tissue where the corresponding miRNA is expressed.
144. The AAV particle of any one of embodiments 135-143, wherein the viral genome comprises at least 1-5 copies of a miR binding site, e.g., at least 1, 2, 3, 4, or 5 copies.
145. The AAV particle of any one of embodiments 135-144, wherein the viral genome comprises at least 3 copies of a miR binding site, optionally wherein all three copies comprise the same miR binding site, or at least one, two, or all of the copies comprise a different miR binding site.
146. The AAV particle of any one of embodiments 135-144, wherein the viral genome comprises at least 4 copies of a miR binding site, optionally wherein all four copies comprise the same miR binding site, or at least one, two, three, or all of the copies comprise a different miR binding site.
147. The AAV particle of any one of embodiments 143-146, wherein the miR binding site comprises a miR122 binding site, a miR183 binding site, a miR-142-3p, or a combination thereof, optionally wherein:
(i) the miR122 binding site comprises the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 3672;
(ii) the miR183 binding site comprises the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 3675; and/or
(iii) the miR-142-3p binding site comprises the nucleotide sequence of SEQ ID NO: 3674, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 3674.
148. The AAV particle of any one of embodiments 135-147, wherein the viral genome is single stranded.
149. The AAV particle of any one of embodiments 135-148, wherein the viral genome further comprises a nucleotide sequence encoding a Rep protein, e.g., a non-structural protein, wherein the Rep protein comprises a Rep78 protein, a Rep68, Rep52 protein, and/or a Rep40 protein.
150. The AAV particle of embodiment 149, wherein the Rep78 protein, the Rep68 protein, the Rep52 protein, and/or the Rep40 protein are encoded by at least one Rep gene.
151. The AAV particle of any one of embodiments 135-150, wherein the viral genome further comprises a nucleic acid sequence encoding the AAV capsid variant of any one of embodiments 1-102 or 108.
152. The AAV particle of any one of embodiments, 162-189, which is isolated, e.g., recombinant.
153. A vector comprising a polynucleotide encoding the AAV capsid variant of any one of embodiments 1-102 or 108, the polynucleotide of any one of embodiments 103-106, 109-123, or a polynucleotide encoding the peptide of embodiment 107.
154. A cell, e.g., a host cell, comprising the AAV capsid variant of any one of embodiments 1-102 or 108, the polynucleotide of any one of embodiments 103-106, 109-123, the peptide of embodiment 107, the AAV particle of any one of embodiments 124-152, or the vector of embodiment 153.
155. The cell of embodiment 154, wherein the cell is a mammalian cell or an insect cell.
156. The cell of embodiment 154 or 155, wherein the cell is a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
157. The cell of embodiment 154 or 155, wherein the cell is a neuron, a sensory neuron, a motor neuron, an astrocyte, or a muscle cell (e.g., a cell of the heart, diaphragm, or quadriceps).
158. A method of making an AAV particle, comprising
(i) providing a host cell comprising a viral genome; and
(ii) incubating the host cell under conditions suitable to enclose the viral genome in the AAV capsid variant of any one of embodiments 1-102 or 108 or an AAV capsid variant encoded by the polynucleotide of any one of embodiments 103-106 or 109-123;
thereby making the AAV particle.
159. The method of embodiment 158, further comprising, prior to step (i), introducing a first nucleic acid molecule comprising the viral genome into the host cell.
160. The method of embodiment 158 or 159, wherein the host cell comprises a second nucleic acid encoding the capsid variant.
161. The method of any one of embodiments 158-160, wherein the second nucleic acid molecule is introduced into the host cell prior to, concurrently with, or after the first nucleic acid molecule.
162. A pharmaceutical composition comprising the AAV particle of any one of embodiments 124-152, an AAV particle comprising the capsid variant of any one of embodiments 1-102 or 108, or an AAV particle comprising the peptide of embodiment 107, and a pharmaceutically acceptable excipient.
163. A method of delivering a payload to a cell or tissue (e.g., a CNS cell, a CNS tissue, a muscle cell, or a muscle tissue), comprising administering an effective amount of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107.
164. The method of embodiment 163, wherein the cell is a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
165. The method of embodiment 163 or 164, wherein the cell is a neuron, a sensory neuron, a motor neuron, an astrocyte, or a muscle cell (e.g., a cell of the heart, diaphragm, or quadriceps).
166. The method of any one of embodiments 163-165, wherein the cell or tissue is within a subject.
167. The method of embodiment 166, wherein the subject has, has been diagnosed with having, or is at risk of having a neurological, e.g., a neurodegenerative disorder.
168. The method of embodiment 166, wherein the subject has, has been diagnosed with having, or is at risk of having a muscular disorder or a neuromuscular disorder.
169. The method of embodiment 166, wherein the subject has, has been diagnosed with having, or is at risk of having a neuro-oncological disorder.
170. The method of embodiment 166, wherein the subject has, has been diagnosed with having, or is at risk of having a genetic disorder, e.g., a monogenic disorder or a polygenic disorder.
171. The method of embodiment 166, wherein the subject has, has been diagnosed with having, or is at risk of having a cardiac disorder, e.g., a cardiac disorder as described herein (e.g., a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction).
172. A method of treating a subject having or diagnosed with having a genetic disorder, e.g., a monogenic disorder or a polygenic disorder, comprising administering an effective amount of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107.
173. A method of treating a subject having or diagnosed with having a neurological disorder, e.g., a neurodegenerative disorder, comprising administering an effective amount of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107.
174. A method of treating a subject having or diagnosed with having a muscular disorder or a neuromuscular disorder, comprising administering an effective amount of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107.
175. A method of treating a subject having or diagnosed with having a cardiac disorder, e.g., a cardiac disorder as described herein (e.g., a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction), comprising administering an effective amount of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107.
176. A method of treating a subject having or diagnosed with having a neuro-oncological disorder, comprising administering an effective amount of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107.
177. The method of any one of embodiments 172-176, where treating comprises prevention of progression of the disease or disorder in the subject.
178. The method of embodiment 166-177, wherein the subject is a human.
179. The method of any one of embodiments 166-178, wherein the AAV particle is administered to the subject intramuscularly, intravenously, intracerebrally, intrathecally, intracerebroventricularly, via intraparenchymal administration, or via intra-cisterna magna injection (ICM).
180. The method of any one of embodiments 166-178, wherein the AAV particle is administered to the subject via focused ultrasound (FUS), e.g., coupled with the intravenous administration of microbubbles (FUS-MB), or MRI-guided FUS coupled with intravenous administration.
181. The method of any one of embodiments 166-180, wherein the AAV particle is administered to the subject intravenously.
182. The method of any one of embodiments 163-181, wherein administration of the AAV particle results in a decreased presence, level, and/or activity of a gene, mRNA, protein, or combination thereof.
183. The method of any one of embodiments 163-181, wherein administration of the AAV particle results in an increased presence, level, and/or activity of a gene, mRNA, protein, or a combination thereof.
184. The pharmaceutical composition of embodiment 162, the AAV particle of any one of embodiments 124-152, an AAV particle comprising the capsid variant of any one of embodiments 1-102 or 108, or an AAV particle comprising the peptide of embodiment 107, for use in a method of delivering a payload to a cell or tissue.
185. The pharmaceutical composition of embodiment 162, the AAV particle of any one of embodiments 124-152, an AAV particle comprising the capsid variant of any one of embodiments 1-102 or 108, or an AAV particle comprising the peptide of embodiment 107, for use in a method of treating a genetic disorder, neurological disorder, neurodegenerative, disorder, muscular disorder, neuromuscular disorder, cardiac disorder, or neuro-oncological disorder.
186. The pharmaceutical composition of embodiment 162, the AAV particle of any one of embodiments 124-152, an AAV particle comprising the capsid variant of any one of embodiments 1-102 or 108, or an AAV particle comprising the peptide of embodiment 107, for use in the manufacture of a medicament.
187. Use of the pharmaceutical composition of embodiment 162, the AAV particle of any one of embodiments 124-152, an AAV particle comprising the capsid variant of any one of embodiments 1-102 or 108, or an AAV particle comprising the peptide of embodiment 107, in the manufacture of a medicament for treating a genetic disorder, a neurological disorder, a neurodegenerative disorder, a muscular disorder, a neuromuscular disorder, a cardiac disorder, or a neuro-oncological disorder.

### BRIEF DESCRIPTION OF THE DRAWINGS

FIG. 1A and FIG. 1B show brain transgene mRNA expression (RT-ddPCR) as fold over TBP (housekeeping gene).

FIG. 2A and FIG. 2B show brain viral DNA biodistribution (ddPCR) as vector genomes per cell.

FIG. 3A and FIG. 3B show brain transgene mRNA expression (RT-ddPCR) as fold over AAV9.

FIG. 4A and FIG. 4B show brain viral DNA biodistribution (ddPCR) as fold over AAV9.

FIG. 5A and FIG. 5B show spinal cord (FIG. 5A) and DRG (FIG. 5B) transgene mRNA expression as fold over TATA box binding protein.

FIG. 6A and FIG. 6B show spinal cord (FIG. 6A) and DRG (FIG. 6B) viral genome biodistribution as vector genomes per cell.

FIG. 7A and FIG. 7B show spinal cord (FIG. 7A) and DRG (FIG. 7B) mRNA expression as fold over AAV9.

FIG. 8A and FIG. 8B show spinal cord (FIG. 8A) and DRG (FIG. 8B) viral genome biodistribution as fold over AAV9.

FIG. 9A, FIG. 9B, FIG. 9C, FIG. 9D, and FIG. 9E show images of the brain transduction profile for TTD-001 and AAV9 as determined by immunohistochemical analyses of the dentate nucleus (FIG. 9A), cerebellar cortex (FIG. 9B), cortex (FIG. 9C), brain stem, hippocampus, thalamus and putamen (FIG. 9D) and dorsal root ganglion (FIG. 9E).

FIG. 10A and FIG. 10B show immunohistochemistry images of the DRG de-targeting characteristic of capsid variant TTD-004, compared to AAV9.

FIG. 11A and FIG. 11B show viral genome biodistribution in peripheral tissues quantified as vector genomes per cell.

FIG. 12A, FIG. 12B, and FIG. 12C show immunohistochemistry images of the heart of a female NHP at day 14 post-intravenous administration of AAV particles comprising a TTD-001 capsid variant, a TTD-004 capsid variant, or a wild-type AAV9 control capsid polypeptide. FIG. 12A provides a series of global images of the heart muscle, FIG. 12B provides a series of images of the left ventricle of the heart, and FIG. 12C provides a series of images of the right ventricle of the heart. For each series of images in FIGS. 12A-12C, the top left panel shows staining following administration of AAV particles comprising a TTD-001 capsid variant, the top right panel shows staining following administration of AAV particles comprising a TTD-004 capsid variant, and the bottom panel shows staining following administration of AAV particles comprising a wild-type AAV9 control capsid variant.

FIGS. 13A-13D show the viral titers by qPCR of TTD-001 with a SEAP/GFP payload after storage for the indicated durations at −80° C. (FIG. 13A), 2-8° C. (FIG. 13B), and room temperature (FIG. 13C), and after the indicated number of freeze thaw cycles (FIG. 13D). FIG. 13E shows collective viral titers by qPCR of TTD-001 with a SEAP/GFP payload for the indicated storage and freeze thaw conditions. FIGS. 13F and 13G show viral titers by qPCR of TTD-001 with a SEAP/GFP payload after storage at −80° C. and 2-8° C., respectively, for up to 90 days.

FIGS. 14A-14D show the % high molecular weight aggregates, as assessed by size exclusion chromatography (SEC), of TTD-001 with a SEAP/GFP payload after storage for the indicated durations at −80° C. (FIG. 14A), 2-8° C. (FIG. 14B), and room temperature (FIG. 14C), and after the indicated number of freeze thaw cycles (FIG. 14D). FIGS. 14E and 14F show the % high molecular weight aggregates, as assessed by SEC, of TTD-001 with a SEAP/GFP payload after storage at −80° C. and 2-8° C., respectively, for up to 90 days.

FIGS. 15A-15D show the % occupancy (e.g., % full capsids), as assessed by size exclusion chromatography-multiangle light scattering (SEC-MALS), of TTD-001 with a SEAP/GFP payload after storage for the indicated durations at −80° C. (FIG. 15A), 2-8° C. (FIG. 15B), and room temperature (FIG. 15C), and after the indicated number of freeze thaw cycles (FIG. 15D). FIGS. 15E and 15F show the % occupancy (e.g., % full capsids), as assessed by SEC-MALS, of TTD-001 with a SEAP/GFP payload after storage at −80° C. and 2-8° C., respectively, for up to 90 days.

FIG. 16A is a series of graph showing the quantification of the copies of viral genomes per diploid cell in various central nervous system and peripheral tissues of NHPs following intravenous administration of varying doses of AAV particles comprising the TTD-001 capsid variant. FIG. 16B is a series of graphs showing the quantification of transgene mRNA relative to the housekeeping gene in various central nervous system and peripheral tissues of NHPs following intravenous administration of varying doses of AAV particles comprising the TTD-001 capsid variant. FIG. 16C is a series of graphs showing the quantification of transgene mRNA relative to the endogenous gene (transgene mRNA as fold change relative to vehicle) in various central nervous system and peripheral tissues of NHPs following intravenous administration of varying doses of AAV particles comprising the TTD-001 capsid variant. FIG. 16D is a series of graphs showing the quantification of protein in the cervical DRG, heart, and liver of NHPs following intravenous administration of varying doses of AAV particles comprising the TTD-001 capsid variant.

FIG. 17A is a graph showing the percentage of transduced cells (% HA positive cells) in various brain regions of NHPs following intravenous administration of a dose of 6.7e12 VG/kg of AAV particles comprising the TTD-001 capsid variant. FIG. 17B is a graph showing the percentage of transduced cells (% HA positive cells) in various brain regions of NHPs following intravenous administration of a dose of 2e13 VG/kg of AAV particles comprising the TTD-001 capsid variant.

FIG. 17C is a graph showing the percentage of neuronal transduction (% HA cells among SMI311+ cells) in the thalamus, dentate nucleus, and spinal cord of the NHPs following intravenous administration of a dose of 2e13 VG/kg of AAV particles comprising the TTD-001 capsid variant.

### DETAILED DESCRIPTION OF THE DISCLOSURE

Described herein, inter alia, are compositions and formulations (e.g., optimized stable formulations) comprising an AAV capsid polypeptide, e.g., an AAV capsid variant, e.g., an AAV capsid variant described herein, and methods of making and using the same. Generally, the AAV capsid variant has enhanced tropism for a cell or tissue, e.g., for the delivery of a payload to said cell or tissue, for example a CNS tissue, a CNS cell, a muscle cell, or a muscle tissue.

In some embodiments, an AAV capsid variant disclosed herein comprises a modification in loop VIII of AAV9, e.g., at positions between 580-599, e.g., at positions 587, 588, 589, and/or 590, numbered relative to SEQ ID NO: 5, 8, 138 or 3636-3647. In some embodiments, loop (e.g., loop VIII) is used interchangeably herein with the term variable region (e.g., variable region VIII), or VR (e.g., VR-VIII). In some embodiments loop VIII comprises positions 580-599 (e.g., amino acids VATNHQSAQAQAQTGWVQNQ (SEQ ID NO: 1195)), numbered according to SEQ ID NO: 138. In some embodiments, loop VIII comprises positions 582-593 (e.g., amino acids TNHQSAQAQAQT (SEQ ID NO: 1196)), numbered according to SEQ ID NO: 138. In some embodiments loop VIII comprises positions 587-593 (e.g., amino acids AQAQAQT (SEQ ID NO: 1197)), numbered according to SEQ ID NO: 138. In some embodiments loop VIII comprises positions 587-590 (e.g., amino acids AQAQ (SEQ ID NO: 4737)), numbered according to SEQ ID NO: 138. In some embodiments, loop VIII or variable region VIII (VR-VIII) is as described in DiMattia et al. “Structural Insights into the Unique Properties of the Adeno-Associated Virus Serotype 9,” Journal of Virology, 12(86):6947-6958 (the contents of which are hereby incorporated by reference in their entirety), e.g., comprising positions 581-593 (e.g., ATNHQSAQAQAQT (SEQ ID NO: 1198)), numbered according to SEQ ID NO: 138.

As demonstrated in the Examples herein below, certain AAV capsid variants described herein show multiple advantages over wild-type AAV9, including (i) increased penetrance through the blood brain barrier following intravenous administration, (ii) wider distribution throughout the multiple brain regions, e.g., frontal cortex, sensory cortex, motor cortex, putamen, thalamus, cerebellar cortex, dentate nucleus, caudate, and/or hippocampus, and/or (iii) elevated payload expression in multiple brain regions. Without wishing to be being bound by theory, it is believed that these advantages may be due, in part, to the dissemination of the AAV capsid variants through the brain vasculature. In some embodiments, the AAV capsids described herein enhance the delivery of a payload to multiple regions of the brain including for example, the frontal cortex, sensory cortex, motor cortex, putamen, thalamus, cerebellar cortex, dentate nucleus, caudate, and/or hippocampus.

Several approaches have been used previously to produce AAV capsids with enhanced tropism for a cell or tissue, e.g., a CNS cell or tissue. One approach used co-infection of cultured cells (Grimm et al., 2008, the contents of which are herein incorporated by reference in its entirety) or in situ animal tissue (Lisowski et al., 2014, the contents of which are herein incorporated by reference in its entirety) with adenovirus, in order to trigger exponential replication of infectious AAV DNA. Another approach involved the use of cell-specific CRE transgenic mice (Deverman et al., 2016. the contents of which are herein incorporated by reference in its entirety) allowing viral DNA recombination specifically in astrocytes, followed by recovery of CRE-recombined capsid variants. Both approaches have had limited success.

The transgenic CRE system used by Deverman et al. (2016) has limited tractable in other animal species and AAV variants selected by directed evolution in mouse tissue do not show similar properties in large animals. Previously described transduction-specific approaches are not amenable to large animal studies because: 1) many tissues of interest (e.g. CNS) are not readily accessible to adenovirus co-infection, 2) the specific adenovirus tropism itself would bias the library distribution, and 3) large animals are typically not amenable to transgenesis or genetic engineering to express CRE recombinase in defined cell types.

To address these limitations, a broadly-applicable functional AAV capsid library screening platform for cell type-specific biopanning in non-transgenic animals has been developed and is described in the appended Examples. In the TRACER (Tropism Redirection of AAV by Cell type-specific Expression of RNA) platform system, the capsid gene is placed under the control of a cell type-specific promoter to drive capsid mRNA expression in the absence of helper virus co-infection. Without wishing to be bound by theory, it is believed that this RNA-driven screen increases the selective pressure in favor of capsid variants which transduce a specific cell type. The TRACER platform allows for generation of AAV capsid libraries whereby specific recovery and subcloning of capsid mRNA expressed in transduced cells is achieved with no need for transgenic animals or helper virus co-infection. Without wishing to be bound by theory, it is believed that since mRNA transcription is a hallmark of full transduction, the methods disclosed herein allow identification of fully infectious AAV capsid mutants, and in addition to its higher stringency, this method allows identification of capsids with high tropism for particular cell types using libraries designed to express CAP mRNA under the control of any cell-specific promoter such as, but not limited to, synapsin-1 promoter (neurons), GFAP promoter (astrocytes), TBG promoter (liver), CAMK promoter (skeletal muscle), MYH6 promoter (cardiomyocytes). Described herein are novel AAV capsid variants generated using the TRACER method which demonstrate enhance tropism in for example a CNS cell, a CNS tissue, a muscle cell, or a muscle tissue.

The AAV particles and payloads of the disclosure may be delivered to one or more target cells, tissues, organs, or organisms. In some embodiments, the AAV particles of the disclosure demonstrate enhanced tropism for a target cell type, tissue or organ. As a non-limiting example, the AAV particle may have enhanced tropism for cells and tissues of the central or peripheral nervous systems (CNS and PNS, respectively), or cells and tissues of a muscle. The AAV particles of the disclosure may, in addition, or alternatively, have decreased tropism for a cell-type, tissue or organ.

In some embodiments, an AAV comprises a small non-enveloped icosahedral capsid virus of the Parvoviridae family and is characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

In some embodiments, AAV are used as a biological tool due to a relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

In some embodiments, the AAV, is a naturally occurring (e.g., wild-type) AAV or a recombinant AAV. In some embodiments, the wild-type AAV vector genome is a linear, single-stranded DNA (ssDNA) molecule approximately 5,000 nucleotides (nt) in length. In some embodiments, inverted terminal repeats (ITRs) cap the viral genome at both the 5′ and the 3′ end, providing origins of replication for the viral genome. In some embodiments, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In some embodiments, the wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are used for replication and packaging, while the capsid proteins are assembled to create the protein shell of the AAV, or AAV capsid polypeptide, e.g., an AAV capsid variant. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In some embodiments, for any one of the amino acid sequences of SEQ ID NOs: 5, 8, or 3636-3647, VP1 comprises amino acids 1-743, VP2 comprises amino acids 138-743, and VP3 comprises amino acids 203-743. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3.

AAV vectors of the present disclosure may be produced recombinantly and may be based on adeno-associated virus (AAV) parent or reference sequences. In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the transduced cell. In some embodiments, the AAV particle of the present disclosure is an scAAV. In some embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

As described herein, the AAV particles of the disclosure comprising an AAV capsid polypeptide, e.g., an AAV capsid variant, and a viral genome, have enhanced tropism for a cell-type or a tissue, e.g., a CNS cell-type, region, or tissue, or a muscle cell-type or tissue.

### Peptides

Disclosed herein are peptides, and associated AAV particles comprising an AAV capsid polypeptide, e.g., an AAV capsid variant, with a peptide, for enhanced or improved transduction of a target tissue (e.g., cells of the CNS or PNS). In some, embodiments, the peptide, is an isolated, e.g., recombinant, peptide. In some embodiments, the nucleic acid encoding the peptide is an isolated, e.g., recombinant nucleic acid.

In some embodiments, the peptide may increase distribution to an AAV particle to a cell, region, or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei.

In some embodiments, the peptide may increase distribution of an AAV particle to a cell, region, or tissue of the PNS. The cell or tissue of the PNS may be, but is not limited to, a dorsal root ganglion (DRG).

In some embodiments, the peptide may increase distribution of an AAV particle to the CNS (e.g., the cortex) after intravenous administration. In some embodiments, the peptide may increase distribution of an AAV particle to the CNS (e.g., the cortex) following focused ultrasound (FUS), e.g., coupled with the intravenous administration of microbubbles (FUS-MB), or MRI-guided FUS coupled with intravenous administration.

In some embodiments, the peptide may increase distribution of an AAV particle to the PNS (e.g., DRG) after intravenous administration. In some embodiments, the peptide may direct an AAV particle to the PNS (e.g., DRG) following focused ultrasound (FUS), e.g., coupled with the intravenous administration of microbubbles (FUS-MB), or MRI-guided FUS coupled with intravenous administration.

In some embodiments, the peptide may increase the distribution of an AAV particle to a cell, region, or tissue of a muscle. In some embodiments, the muscle is a heart muscle. In some embodiments, the peptide may increase distribution of an AAV particle to a muscle cell, region, or tissue after intravenous administration.

A peptide may vary in length. In some embodiments, the peptide is about 3 to about 20 amino acids in length. As non-limiting examples the peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length. In some embodiments, a peptide comprises about 6 to 12 amino acids in length, e.g., about 9 amino acids in length. In some embodiments, a peptide comprises about 5 to 10 amino acids in length, e.g., about 7 amino acids in length. In some embodiments, a peptide comprises about 15 to 20 amino acids in length, e.g., about 19 amino acids in length.

In some embodiments a peptide may comprise a sequence as set forth in Table 1A. In some embodiments a peptide may comprise a sequence as set forth in Table 1B. In some embodiments a peptide may comprise a sequence as set forth in Table 2. In some embodiments a peptide may comprise a sequence as set forth in Table 7. In some embodiments a peptide may comprise a sequence as set forth in Table 10. In some embodiments a peptide may comprise a sequence as set forth in Table 11. In some embodiments a peptide may comprise a sequence as set forth in Table 20. In some embodiments, the peptide comprises an amino acid sequence of any one of peptide 1, 2, 3, 4, 5, 6, 7, 8, 9, 10, 11, or 12, e.g., as described in Table 2. In some embodiments, the peptide is isolated, e.g., recombinant.

TABLE 1A
Exemplary Peptide Sequences

| | SEQ | | SEQ | | SEQ | | SEQ |
|---|---|---|---|---|---|---|---|
| | ID | | ID | Peptide | ID | Peptide | ID |
| Peptide Sequence | NO: | Peptide Sequence | NO: | Sequence | NO: | Sequence | NO: |
| QLNGAVHLYAQ | 139 | PLNGAVHLYA | 224 | PLNGAVHLYA | 309 | PLNGAVHLYA | 394 |
| AQLSPVQN | | QAQTLQVQN | | QAQTGWVRN | | QAQTGSVQI | |
| PLDGAVHLYAQ | 140 | PLNGAVHLYA | 225 | PLNGAVHLYA | 310 | PLNGTVHLYA | 395 |
| PQTGWVQN | | QAQTMSVQN | | QAQLAAVQN | | QAQTGGVQN | |
| QLNGAVHLYAQ | 141 | QLNGAVHLYA | 226 | PLNGAVHLYA | 311 | PLNGAVHLYA | 396 |
| AQTMSVQN | | QAQLQPVQN | | QAQTSPDQN | | KAQVSPVQN | |
| PLDSSVHLYAQ | 142 | PLNGAVHLYA | 227 | PLNGAVHLYA | 312 | PLNGAVHLYA | 397 |
| AQTGWVQN | | QAQVAKVQN | | QAQRSGVQN | | QAQRGGVQN | |
| PLNGAVHLYAQ | 143 | PLNGAVHLYA | 228 | PLNGAVHLYA | 313 | PLNGGVHLYA | 398 |
| AQTTKVQN | | QAQRAAVQN | | QAQTGGVQT | | QAQTGLVQN | |
| PLDGAVHLYAQ | 144 | PLNGAVHLYA | 229 | PLNGAVHLYA | 314 | PLNGAVHLYA | 399 |
| AQTGSVQN | | QAQTVGVQN | | QAQTGWVPN | | QAQVMSVQN | |
| ALNGAVHLYAQ | 145 | PLNGAVHLYA | 230 | PLDSAVHLYA | 315 | PLNGAVHLYA | 400 |
| AQTTSVQN | | QAQLNPVQN | | QAQTGWVQN | | QAQTTGVQN | |
| PLNGSVHLYAQ | 146 | PLNGAVHLYA | 231 | PLNGAVHLYA | 316 | PLNGAVHLYA | 401 |
| AQTMSVQN | | QAQLSQVQN | | QAQTTPVQT | | QAQLSPVQK | |
| QLNGAVHLYAQ | 147 | PLNGTVHLYAQ | 232 | PLNGAVHLYA | 317 | SLNGAVHLYA | 402 |
| AQTSPVQN | | AQTGSVQN | | QAQLMAVQN | | QAQTAAVQN | |
| PLNGAVHLYAQ | 148 | PLNGAVHLYA | 233 | PLNGAVHLYA | 318 | PINGAVHLYA | 403 |
| AQTMKVQN | | QAQTKPVQN | | QAQTMPVQN | | QAQTSPVQN | |
| PLNGAVHLYAQ | 149 | PLNGAVHLYA | 234 | PLNGAVHHYA | 319 | PLNGAVHLYA | 404 |
| AQVAQVQN | | QAQTNAVQN | | QAQTSPVQN | | QAQLSRVQN | |
| PLDGAVHLYAQ | 150 | ALDGAVHLYA | 235 | PLNGAVHLYA | 320 | PLNGAVHLYA | 405 |
| AQTGGVQN | | QAQTGWVQN | | QAQLANVQN | | QAQVSSVQK | |
| PLNGAVHLYAQ | 151 | PLNGAVHLYA | 236 | PLNGAVHLYA | 321 | PLNGAVHLYA | 406 |
| AQTAWDQN | | QAQLATVQN | | QAQVSTVQN | | QAQTAPVQT | |
| PLNGSVHLYAQ | 152 | PLNGAVHLYA | 237 | PLNGAVHLYA | 322 | PLNGAVHLYA | 407 |
| AQTGWDQN | | QAQVTPVQN | | QAQSAQVQN | | QAQMAPVQN | |
| PLNGAVHLYAQ | 153 | PLNGAVHLYA | 238 | PLNGAVHLYA | 323 | PLNGAVHLYA | 408 |
| AQTGSVQH | | QAQVQAVQN | | QAQNTPVQN | | QAQILGVQN | |
| PLNGAVHLYAQ | 154 | PLNGAVHLYA | 239 | PLNGAVHLYA | 324 | PLNGAVHLYA | 409 |
| AQVKQVQN | | QAQTTSVQN | | QAQVSSVQT | | QAQTASVQT | |
| PLNGAVHLYAQ | 155 | PLNGAVHLYA | 240 | PLNGAVHLYA | 325 | PLNGAVHLYA | 410 |
| AQSAPVQN | | QAQCTPVQN | | QAQTVSVKN | | QAQTGSLQN | |
| PLNGAVHLYAQ | 156 | ALNGAVHLYA | 241 | PLNGAVHLYA | 326 | PLNGAVHLYA | 411 |
| AQLSKVQN | | QAQTGRVQN | | QPQTGLVQN | | QAQTGTVQN | |
| PLNGAVHLYAQ | 157 | PLNGSVHLYAQ | 242 | PLNGAVHLYA | 327 | PLNGGVHLYA | 412 |
| AQLAPVQN | | AQTGWVQD | | QAQTGSVQN | | QAQTGWVQN | |
| PLNGAVHLYAQ | 158 | PLNGAVHLYA | 243 | SLNGAVHLYA | 328 | PLNGAVHLYA | 413 |
| AQLAQVQN | | QAQTAGVQN | | QAQTLPVQN | | QAQTGSVQT | |
| QLNGAVHLYAQ | 159 | PLNGAVHLYA | 244 | PLNGAVHLYA | 329 | PLNGAVHLYA | 414 |
| AQVASVQN | | QAQTSQVQN | | QAQTGGAQN | | QAQTSSVQT | |
| PLNGAVHLYAQ | 160 | PLNGAVHLYA | 245 | SLNGAVHLYA | 330 | PLNGAVHLYA | 415 |
| AQTAKVQN | | QAQTMNVQN | | QAQTAQVQN | | QAQTSHVQN | |
| PLNGAVHLYAQ | 161 | PLNGAVHLYA | 246 | PLNGAVHLYA | 331 | PLNGGVHLYA | 416 |
| AQSAKVQN | | QAQTSTVQN | | QAQTAVVQN | | QAQTGWDQN | |
| PLNGAVHLYAQ | 162 | PLNGAVHLYA | 247 | PLNGAVHLYA | 332 | PLNGAVHLYA | 417 |
| AQTGCFQN | | QAQVKPVQN | | QAQRLGVQN | | QAQRIGVQN | |
| PLNGAVHLYAQ | 163 | PLNGAVHLYA | 248 | PLNGSVHLYA | 333 | TLNGAVHLYA | 418 |
| AQTQKVQN | | QAQASPVQN | | QAQTGWVQH | | QAQTTSVQN | |
| PLNGSVHLYAQ | 164 | PLNGAVHLYA | 249 | PLNGAVHLYA | 334 | PLNGAVHLYA | 419 |
| AQTTSVQN | | QAQVAAVQN | | QAQRTLVQN | | QAQTGWGQT | |
| PLNGGVHLYAQ | 165 | PLNGAVHLYA | 250 | SLNGAVHLYA | 335 | PLNGAVHLYA | 420 |
| AQTGRVQN | | QAQLKSVQN | | QAQTMAVQN | | QAQSAMVQN | |
| PLNGAVHLYAQ | 166 | PLNGAVHLYA | 251 | PLNGAVHLYA | 336 | PLNGAVHLYA | 421 |
| AQTVAVQN | | QAQIAAVQN | | QAQTQMVQN | | QAQTSMVQN | |
| ALNGAVHLYAQ | 167 | PLNGAVHLYA | 252 | PLNGAVHLYA | 337 | PLNGAVHLYA | 422 |
| AQSSPVQN | | QAQTAAVQN | | QAQITPVQN | | QAQSMGVQN | |
| PLNGAVHLYAQ | 168 | PLNGAVHLYA | 253 | PLNGAVHLYA | 338 | PLNGAVHLYA | 423 |
| AQLSPVQN | | QAQTKAVQN | | QAQTVWVQK | | QAQSMSVQN | |
| QLNGAVHLYAQ | 169 | PLNGAVHLYA | 254 | PLNGAVHLYA | 339 | PLNGAVHLYA | 424 |
| AQTTSVQN | | QAQTGSVQS | | QAQRSAVQN | | QAQTSSVQN | |
| PLNGAVHLYAQ | 170 | PLNGAVHLYA | 255 | PLNGAVHLYA | 340 | PLNGAVHLYA | 425 |
| AQTTQVQN | | QAQVSNVQN | | QAQTASVQN | | QAQTSGVQN | |
| PLNGAVHLYAQ | 171 | PLNGAVHLYA | 256 | PLNGAVHLYA | 341 | PLNGAVHLYA | 426 |
| AQTAQVQN | | QAQTAPVQN | | QAQTMGVQN | | QAQTGAVQN | |
| QLNGAVHLYAQ | 172 | PLNGAVHLYA | 257 | PLNGAVHLYA | 342 | PLNGAVHLYA | 427 |
| AQTVAVQN | | QAQLMPVQN | | QAQTGGVQH | | QAQVNSVQN | |
| PLNGAVHLYAQ | 173 | PLNGAVHLYA | 258 | PLNGAVHLYA | 343 | PLNGAVHLYA | 428 |
| AQRIAVQN | | QAQLHPVQN | | QAQVQSVQN | | QAQVAGVQN | |
| PLNGAVHLYAQ | 174 | PLNGAVHLYA | 259 | PLNGAVHLYA | 344 | PLNGAVHLYA | 429 |
| AQRASVQN | | QAQRAQVQN | | QAQTGGVQN | | QAQIGSVQN | |
| PLNGAVHLYAQ | 175 | PLNGAVHLYA | 260 | SLNGAVHLYA | 345 | PLDGAVHVYA | 430 |
| AQTTPVQN | | QAQLTNVQN | | QAQTAPVQN | | QAQTGWVQN | |
| QLNGAVHLYAQ | 176 | PLNGAVHLYA | 261 | PLNGAVHLYA | 346 | TLNGAVHLYA | 431 |
| AQLASVQN | | QAQRTTVQN | | QAQISPVQN | | QAQTTPVQN | |
| PLNGAVHLYAQ | 177 | PLNGAVHLYA | 262 | PLNGAVHLYA | 347 | PLNGAVHLYA | 432 |
| AQLTPVQN | | QAQTSVVQN | | QPQTGWVKN | | QAQLGSVQN | |
| PLNGAVHLYAQ | 178 | SLNGAVHLYA | 263 | PLNGAVHLYA | 348 | PLNGAVHLYA | 433 |
| AQSTPVQN | | QAQTMSVQN | | QAQTGSAQN | | QAQVNGVQN | |
| PLNGAVHLYAQ | 179 | ALNGAVHLYA | 264 | PLNGAVHLYA | 349 | QLNGAVHLYA | 434 |
| AQTSPVQN | | QAQTLAVQN | | QAQTGWAQN | | QAQLSSVQN | |
| QLNGAVHLYAQ | 180 | PLNGAVHLYA | 265 | PLNGAVHLYA | 350 | PLNGAVHLYA | 435 |
| AQVSQVQN | | QAQRMSVQN | | QAQTMSVQT | | QAQLTAVQN | |
| PLNGAVHLYAQ | 181 | ALNGAVHLYA | 266 | PLNGSVHLYA | 351 | PLNGAVHLYA | 436 |
| AQTMQVQN | | QAQLTPVQN | | QAQTAWVQN | | QAQVQNVQN | |
| PLNGAVHLYAQ | 182 | PLNGAVHLYA | 267 | PLNGAVHLYA | 352 | PLNGAVHLYA | 437 |
| AQTSKVQN | | QAQVGNVQN | | QAQVGGVQN | | QAQTKSVQN | |
| SLNGAVHLYAQ | 183 | PLNGAVHLYA | 268 | PLNGAVHLYA | 353 | PLNGAVHLYA | 438 |
| AQVSPVQN | | QAQLMQVQN | | KAQTSPVQN | | QAQSVGVQN | |
| PLNGAVHLYAQ | 184 | PLNGSVHLYAQ | 269 | PLNGAVHLYA | 354 | QLNGAVHLYA | 439 |
| AQVSQVQN | | AQTAQVQN | | QAQLAPVQT | | QAQLGPVQN | |
| PLNGAVHLYAQ | 185 | PLNGAVHLYA | 270 | PLDGAVHLYA | 355 | PLNGAVHLYA | 440 |
| AQVSPVQN | | QAQTATVQN | | QAQTALVQN | | QAQTAWVQT | |
| PLNGAVHLYAQ | 186 | PLNGAVHLYA | 271 | PLNGAVHLYA | 356 | PLNGAVHLYA | 441 |
| AQTVQVQN | | QAQVHPVQN | | QAQTALVQN | | QAQNASVQN | |
| PLNGAVHLYAQ | 187 | PLNGAVHLYA | 272 | PLNGAVHLYA | 357 | PLNGAVHLYA | 442 |
| AQVTAVQN | | QAQVSPVQT | | QAQLAGVQN | | QAQTSAVQN | |
| PLNGAVHLYAQ | 188 | PLNGAVHLYA | 273 | PLNGAVHLYA | 358 | ALNGAVHLYA | 443 |
| AQRQPVQN | | QAQISSVQN | | QAQRTAVQN | | QAQLAAVQN | |
| PLNGNVHLYAQ | 189 | PLNGAVHLYA | 274 | PLNGAVHLYA | 359 | PLNGAVHLYD | 444 |
| AQTGGVQN | | QAQVASVQN | | QAQRSPVQN | | QAQVSPVQN | |
| PLDGAVHLYAQ | 190 | PLNGAVHLYA | 275 | PLNGAVHLYA | 360 | PLNGAVHLYA | 445 |
| AQTAWVQN | | QAQTRWDQN | | QAQTLAVQN | | QAQTMSVKN | |
| PLNGAVHLYAQ | 191 | PLNGAVHLYA | 276 | PLNGAVHLYA | 361 | PLNGAVHLYA | 446 |
| AQISGVQN | | QAQTMTVQN | | QAQLAHVQN | | QAQTANVQN | |
| PLNGAVHLYAQ | 192 | PLNGAVHLYA | 277 | PLNGAVHLYA | 362 | PLNGAVHLYA | 447 |
| AQVRPVQN | | QAQRSSVQN | | QAQTSLVQN | | QAQTGWFQN | |
| PLNGAVHLYAQ | 193 | PLNGGVHLYA | 278 | PLNGAVHLYA | 363 | PLNGNVHLYA | 448 |
| AQLGPVQN | | QAQTGWVRN | | QAQRLSVQN | | QAQTGWVQN | |
| PLNGAVHLYAQ | 194 | PLNGAVHLYA | 279 | PLNGAVHLYD | 364 | PLNGSVHLYA | 449 |
| AQTNQVQN | | QAQTAWVQN | | QAQLSPVQN | | QAQTGWVQS | |
| PLNGAVHLYAQ | 195 | PLNGSVHLYAQ | 280 | PLNGAVHLYA | 365 | PLNGAVHLYA | 450 |
| AQVQQVQN | | AQPGWVQN | | QAQLMGVQN | | QAQTGGVLN | |
| PLNGAVHLYAQ | 196 | ALNGAVHLYA | 281 | PLNGSVHLYA | 366 | PLNGAVHLYA | 451 |
| AQVANVQN | | QAQTGWAQN | | QAQTGWVQT | | QAQLTSVQN | |
| PLNGAVHLYAQ | 197 | PLNGAVHLYA | 282 | PLNGAVHLYA | 367 | PLNGAVHLYA | 452 |
| AQAAPVQN | | QAQRTGVQN | | QAQSMQVQN | | QAQTGWVQT | |
| QLNGAVHLYAQ | 198 | ALNGAVHLYA | 283 | PLNGGVHLYA | 368 | PLNGAVHLYA | 453 |
| AQVAQVQN | | QAQTGWVQS | | QAQTGWFQN | | QAQRSQVQN | |
| PLNGAVHLYAQ | 199 | PLNGAVHLYA | 284 | PLNGAVHLYA | 369 | PLNGAVHIYA | 454 |
| AQRSTVQN | | QAQVATVQN | | QAQTQTVQN | | QAQVSPVQN | |
| PLNGAVHLYAQ | 200 | PLNGAVHLYA | 285 | PLDGAVHLYS | 370 | PLNGSVHLYA | 455 |
| AQTMAVQN | | QAQVTSVQN | | QAQTGWVQN | | QAQTGWFQN | |
| PLNGAVHLYAQ | 201 | PLNGAVHLYA | 286 | PLNGAVHLYA | 371 | PLNGAVHLYA | 456 |
| AQIQPVQN | | QAQVSSVQN | | QAQTGWEQN | | QAQRIPVQN | |
| PLNGAVHLYAQ | 202 | PLNGSVHLYAQ | 287 | PLNGAVHLYA | 372 | QLNGAVHLYA | 457 |
| AQIASVQN | | AQTSSVQN | | QAQVGSVQN | | QAQLVPVQN | |
| PLNGAVHLYAQ | 203 | PLNGAVHLYA | 288 | PLNGAVHLYA | 373 | PLNGAVHLYA | 458 |
| AQTVSVQN | | QAQTNSVQN | | QAQVSGVQN | | QAQTGWVQD | |
| PLNGAVHLYAQ | 204 | PLNGAVHLYA | 289 | PLNGAVHLYA | 374 | PLNGAVHLYA | 459 |
| AQRGSVQN | | QAQVKAVQN | | QAQVMAVQN | | KAQVSSVQN | |
| PLNGAVHLYAQ | 205 | PLNGAVHLYA | 290 | PLNGAVHLYA | 375 | PLNGAVHLYA | 460 |
| AQNSPVQN | | QAQSGPVQN | | QAQIGGVQN | | QAQRVEVQN | |
| PLNGAVHLYAQ | 206 | PLNGAVHLYA | 291 | PLNGAVHLYA | 376 | PLNGAVHLYA | 461 |
| AQLQPVQN | | QAQTGPVQN | | QAQIAGVQN | | QAQVLPVQN | |
| PLNGAVHLYAQ | 207 | PLNGAVHLYA | 292 | PLNGAVHLYA | 377 | PLNGSVHLYA | 462 |
| AQVTGVQN | | QAQTAMVQN | | KAQTVSVQN | | QAQTGWVRN | |
| PLNGAVHLYAQ | 208 | PLNGAVHLYA | 293 | PLNGAVHLYS | 378 | PLNGTVHLYA | 463 |
| AQVMQVQN | | QAQTQPVQN | | QAQTGRVQN | | QAQSSPVQN | |
| PLNGAVHLYAQ | 209 | PLNGSVHLYAQ | 294 | PLNGAVHLYA | 379 | PLNGAVHLYA | 464 |
| AQSMAVQN | | AQTGSVQN | | QAQLSHVQN | | QAQTGGVQK | |
| PLNGAVHLYAQ | 210 | PLNGAVHLYA | 295 | PLNGAVHLYA | 380 | PLNGAVHLYA | 465 |
| AQVGKVQN | | QAQTQQVQN | | QAQVQTVQN | | QAQSVAVQN | |
| ALNGAVHLYAQ | 211 | PLNGAVHLYA | 296 | SLDGAVHLYA | 381 | QLNGAVHLYA | 466 |
| AQSAPVQN | | QAQVSRVQN | | QAQTGWVQN | | QAQLSGVQN | |
| PLNGAVHLYAQ | 212 | QLNGAVHLYA | 297 | PLNGAVHLYA | 382 | PLNGAVHLYA | 467 |
| AQIQSVQN | | QAQVLPVQN | | QPQTGWDQN | | QAQLQQVQN | |
| PLNGAVHLYAQ | 213 | PLNGAVHLYA | 298 | PLNGAVHLYA | 383 | PLNGAVHLYA | 468 |
| AQCSPVQN | | QAQTGWVQP | | QAQRNSVQN | | QAQLSTVQN | |
| PLNGAVHLYAQ | 214 | QLNGAVHLYA | 299 | PLNGAVHLYA | 384 | PLNGAVHLYA | 469 |
| AQLQRVQN | | QAQLGSVQN | | QAQTLPVQN | | QAQSAAVQN | |
| PLNGAVHLYAQ | 215 | PLNGAVHLYA | 300 | PLNGAVHLYA | 385 | SLNGAVHLYA | 470 |
| AQTAWVQH | | QAQVSAVQN | | QAQTKQVQN | | QAQVSAVQN | |
| PLDGAVHLYAQ | 216 | PLNGAVHLYA | 301 | PLNGSVHLYA | 386 | PLNGAVHLYA | 471 |
| AQTGWDQN | | QAQVLSVQN | | QAQTGWVQN | | QAQRTSVQN | |
| PLNGAVHLYAQ | 217 | PLNGAVHLYA | 302 | PLNGAVHLYA | 387 | PLNGAVHLYA | 472 |
| AQTPPVQN | | QAQTQHVQN | | QAQLGQVQN | | QAQTQNVQN | |
| PLNGAVHLYAQ | 218 | PLNGAVHLYA | 303 | SLNGAVHLYA | 388 | PLNGAVHLYA | 473 |
| AQVTKVQN | | QAQLASVQN | | QAQTGRVQN | | QAQVNAVQN | |
| PLNGAVHLYAQ | 219 | PLNGAVHLYA | 304 | PLDGAVHLYA | 389 | PLNGAVHLYA | 474 |
| AQSSPVQN | | QAQQAPVQN | | QAQTGWVQN | | QAQTVAVQT | |
| PLNGAVHLYAQ | 220 | PLNGAVHLYA | 305 | PLNGSVHLYA | 390 | PLNGAVHLYA | 475 |
| AQAGPVQN | | QAQNAQVQN | | QAQTGWAQN | | QAQLMSVQN | |
| PLNGAVHLYAQ | 221 | PLNGAVHLYA | 306 | TLNGAVHLYA | 391 | PLNGAVHLYA | 476 |
| AQLARVQN | | QAQATPVQN | | QAQVASVQN | | QAQTGWVQN | |
| PLNGAVHLYAQ | 222 | PLNGAVHLYA | 307 | PLNGAVHLYA | 392 | PLNGAVHLYA | 566 |
| AQTTTVQN | | QAQVQPVQN | | QAQNMQVQN | | QAQLSPVKN | |
| PLNGAVHLYAQ | 223 | PLNGAVHLYA | 308 | PLNGAVHLYA | 393 | | |
| AQTGGFQN | | QAQTTAVQN | | QAQNVQVQN | | | |

TABLE 1B
Exemplary Peptide Sequences

| | SEQ | | SEQ | | SEQ | | SEQ |
|---|---|---|---|---|---|---|---|
| Peptide | ID | Peptide | ID | Peptide | ID | Peptide | ID |
| Sequence | NO: | Sequence | NO: | Sequence | NO: | Sequence | NO: |
| PLNGAVHLYAQA | 476 | PLNGAVHLYAQA | 423 | PLNGAVHLYAQ | 369 | PLNGAVHLYAQ | 478 |
| QTGWVQN | | QSMSVQN | | AQTQTVQN | | AQTGWVQH | |
| SLNGAVHLYAQA | 1036 | PLNGAVHLYAQA | 646 | PLNGAVHLYAQ | 427 | PLNGAVHLYAQ | 797 |
| QTGWVQN | | QSQLVQN | | AQVNSVQN | | AQTGWGQN | |
| QLNGAVHLYAQA | 680 | PLNGAVHLYAQA | 429 | PLNGAVHLYAQ | 339 | PLNGAVHLYAQ | 768 |
| QTGWVQN | | QIGSVQN | | AQRSAVQN | | AQTGWVQI | |
| ALNGAVHLYAQA | 577 | PLNGAVHLYAQA | 488 | PLNGAVHLYAQ | 365 | PLNGAVHLYAQ | 379 |
| QTGWVQN | | QRSVVQN | | AQLMGVQN | | AQLSHVQN | |
| PLNGSVHLYAQA | 386 | PLNGAVHLYAQA | 252 | PLNGAVHLYAQ | 509 | PLNGAVHLYAQ | 545 |
| QTGWVQN | | QTAAVQN | | AQRQSVQN | | AQSAFVQN | |
| PVNGAVHLYAQA | 1019 | PLNGAVHLYAQA | 178 | PLNGAVHLYAQ | 711 | PLNGAVHLYAQ | 228 |
| QTGWVQN | | QSTPVQN | | AQLVGVQN | | AQRAAVQN | |
| PLNGAVHLYAQA | 911 | PLNGAVHLYAQA | 231 | PLNGAVHLYAQ | 187 | PLNGAVHLYAQ | 201 |
| QTGLVQN | | QLSQVQN | | AQVTAVQN | | AQIQPVQN | |
| PLNGAVHLYAQA | 327 | PLNGGVHLYAQA | 398 | PLNGAVHLYAQ | 261 | PLDGAVHLYAQ | 190 |
| QTGSVQN | | QTGLVQN | | AQRTTVQN | | AQTAWVQN | |
| PLNGAVHLYSQA | 924 | PLNGAVHLYAQA | 171 | PLNGAVHLYAQ | 422 | PLNGAVHLYAQ | 408 |
| QTGWVQN | | QTAQVQN | | AQSMGVQN | | AQILGVQN | |
| PLNGGVHLYAQA | 412 | PLNGAVHLYAQA | 291 | PLNGAVHLYAQ | 374 | PLNGAVHLYAQ | 433 |
| QTGWVQN | | QTGPVQN | | AQVMAVQN | | AQVNGVQN | |
| PLNGTVHLYAQA | 557 | PLNGAVHLYSQA | 573 | PLNGAVHLYAQ | 233 | PLNGAVHLYAQ | 547 |
| QTGWVQN | | QTGLVQN | | AQTKPVQN | | AQSVQVQN | |
| PLNGAVHLYAQA | 344 | ALNGAVHLYAQA | 766 | PLNGAVHLYAQ | 322 | PLNGAVHLYAQ | 755 |
| QTGGVQN | | QTGGVQN | | AQSAQVQN | | AQTGWVHN | |
| PLDGAVHLYAQA | 389 | PLNGAVHLYAQA | 248 | PLNGAVHLYAQ | 205 | PLNGAVHLYAQ | 156 |
| QTGWVQN | | QASPVQN | | AQNSPVQN | | AQLSKVQN | |
| PLNGAVHLYAQA | 279 | PLNGAVHLYAQA | 373 | PLNGAVHLYAQ | 306 | PLNGAVHLYAQ | 675 |
| QTAWVQN | | QVSGVQN | | AQATPVQN | | AQTNGVQN | |
| PLNGAVHLYAQP | 505 | PLNGTVHLYAQA | 970 | PLNGQVHLYAQ | 718 | PLNGAVHLYAQ | 552 |
| QTGWVQN | | QTGLVQN | | AQTGWVQN | | AQRTQVQN | |
| PLNGAVHLYAQA | 528 | PLNGAVHLYAQA | 155 | PLNGAVHLYAQ | 428 | PLNGAVHLYAQ | 245 |
| QTGRVQN | | QSAPVQN | | AQVAGVQN | | AQTMNVQN | |
| PLNGAVHLYAQA | 340 | PLNGAVHLYAQA | 224 | PLNGAVHLYAQ | 204 | PLNGAVHLYAQ | 397 |
| QTASVQN | | QTLQVQN | | AQRGSVQN | | AQRGGVQN | |
| QLNGSVHLYAQA | 622 | PLNGAVHLYAQA | 206 | PLNGAVHLYAQ | 247 | PLNGAVHLYAQ | 570 |
| QTGWVQN | | QLQPVQN | | AQVKPVQN | | AQTTRVQN | |
| PLNGAVHLYAQA | 605 | PLNGAVHLYAQA | 270 | PLNGSVHLYAQ | 351 | PLNGAVHLYAQ | 192 |
| QLSSVQN | | QTATVQN | | AQTAWVQN | | AQVRPVQN | |
| PLNGAVHLYAQA | 424 | PLNGAVHLYAQA | 666 | PLNGAVHLYAQ | 265 | PLNGAVHLYAQ | 289 |
| QTSSVQN | | QTGQVQN | | AQRMSVQN | | AQVKAVQN | |
| SLNGAVHLYAQA | 707 | PLNGAVHLYAQA | 596 | PLNGAVHLYAQ | 594 | PLNGAVHLYAQ | 221 |
| QTGLVQN | | QATSVQN | | AQNLPVQN | | AQLARVQN | |
| PLNGAVHLYAQA | 821 | PLNGTVHLYAQA | 232 | PLNGAVHLYAQ | 619 | PLNGAVHLYAQ | 523 |
| QSSLVQN | | QTGSVQN | | AQNALVQN | | AQNQPVQN | |
| PLNGAVHLYAQA | 1077 | PLNGAVHLYAQA | 375 | PLNGAVHLYAQ | 492 | PLNGAVHLYAQ | 148 |
| QSSSVQN | | QIGGVQN | | AQRTPVQN | | AQTMKVQN | |
| PLNGAVHLYAQA | 593 | PLNGAVHLYAQA | 249 | PLNGAVHLYAQ | 627 | PLNGAVHLYAQ | 592 |
| QTLSVQN | | QVAAVQN | | AQRQLVQN | | AQTYAVQN | |
| PLNGAVHLYAQA | 203 | PLNGGVHLYAQA | 759 | PLNGAVHLYAQ | 495 | QLNGNVHLYAQ | 540 |
| QTVSVQN | | QTGSVQN | | AQVQGVQN | | AQTGWVQN | |
| PLNGAVHLYAQA | 286 | PLNGAVHLYAQA | 421 | PLNGAVHLYAQ | 207 | PLNGAVHLYAQ | 163 |
| QVSSVQN | | QTSMVQN | | AQVTGVQN | | AQTQKVQN | |
| PLNGNVHLYAQA | 448 | PLNGAVHLYAQA | 1027 | PLNGAVHLYAQ | 321 | PLNGAVHLYAQ | 143 |
| QTGWVQN | | QTVWVQN | | AQVSTVQN | | AQTTKVQN | |
| PLNGAVHLYAQA | 179 | PLNGAVHLYAQA | 292 | PLNGAVHLYAQ | 441 | PLNGAVHLYAQ | 376 |
| QTSPVQN | | QTAMVQN | | AQNASVQN | | AQIAGVQN | |
| QLNGAVHLYAQA | 856 | PLNGAVHLYAQA | 426 | PLNGAVHLYAQ | 460 | PLNGAVHLYAQ | 472 |
| QTGLVQN | | QTGAVQN | | AQRVEVQN | | AQTQNVQN | |
| PLNGAVHLYAQA | 185 | PLNGAVHLYAQA | 284 | PLNGAVHLYAQ | 740 | PLNGAVHLYAQ | 361 |
| QVSPVQN | | QVATVQN | | AQATGVQN | | AQLAHVQN | |
| PLNGAVHLYAQA | 225 | PLNGAVHLYAQA | 500 | PLNGAVHLYAQ | 945 | PLNGAVHLYAQ | 586 |
| QTMSVQN | | QQSPVQN | | AQAMSVQN | | AQNTQVQN | |
| PLNGAVHLYAQA | 168 | PLNGAVHLYAQA | 612 | PLNGAVHLYAQ | 383 | PLNGAVHLYAQ | 493 |
| QLSPVQN | | QTQAVQN | | AQRNSVQN | | AQRQQVQN | |
| PLNGAVHLYAQA | 274 | PLNGAVHLYAQA | 238 | PLNGAVHLYAQ | 208 | PLNGAVHLYAQ | 447 |
| QVASVQN | | QVQAVQN | | AQVMQVQN | | AQTGWFQN | |
| SLDGAVHLYAQA | 381 | PLNGAVHLYAQA | 359 | PLNGAVHLYAQ | 367 | PLNGAVHLYAQ | 259 |
| QTGWVQN | | QRSPVQN | | AQSMQVQN | | AQRAQVQN | |
| PLNGAVHLYAQA | 356 | PLNGAVHLYAQA | 236 | PLNGAVHLYAQ | 467 | PLNGAVHLYAQ | 385 |
| QTALVQN | | QLATVQN | | AQLQQVQN | | AQTKQVQN | |
| PLNGAVHLYAQA | 239 | PLNGAVHLYAQA | 149 | PLNGAVHLYAQ | 341 | PLNGAVHLYAQ | 302 |
| QTTSVQN | | QVAQVQN | | AQTMGVQN | | AQTQHVQN | |
| PLNGAVHLYAQA | 384 | PLNGAVHLYAQA | 310 | PLNGAVHLYAQ | 387 | PLNGAVHLYAQ | 587 |
| QTLPVQN | | QLAAVQN | | AQLGQVQN | | AQTGWLQN | |
| HLNGAVHLYAQA | 665 | PLNGAVHLYAQA | 765 | PLDGAVHLYAQ | 144 | PLNGAVHLYAQ | 194 |
| QTGWVQN | | QTGWVKN | | AQTGSVQN | | AQTNQVQN | |
| PLNGAVHLYAQA | 461 | PLNGAVHLYAQA | 199 | PLDSAVHLYAQ | 315 | PLNGAVHLYAQ | 268 |
| QVLPVQN | | QRSTVQN | | AQTGWVQN | | AQLMQVQN | |
| PLNGAVHLYAQA | 817 | ALDGAVHLYAQA | 235 | PLNGAVHLYAQ | 415 | PLNGAVHLYAQ | 473 |
| QRGWVQN | | QTGWVQN | | AQTSHVQN | | AQVNAVQN | |
| PLNGAVHLYAQA | 689 | PLNGAVHLYAQA | 334 | PLNGAVHLYAQ | 197 | PLNGAVHLYAQ | 380 |
| QLSGVQN | | QRTLVQN | | AQAAPVQN | | AQVQTVQN | |
| PLNGAVHLYAQA | 303 | PLNGAVHLYAQA | 524 | PLNGAVHLYAQ | 453 | PLNGAVHLYAQ | 503 |
| QLASVQN | | QLGTVQN | | AQRSQVQN | | AQTQRVQN | |
| PLNGAVHLYAQA | 219 | PLNGAVHLYAQA | 475 | PLNGAVHLYAQ | 832 | PLNGAVHLYAQ | 210 |
| QSSPVQN | | QLMSVQN | | AQTYSVQN | | AQVGKVQN | |
| PLNGAVHLYAQA | 551 | PLNGAVHLYAQA | 193 | PLNGAVHLYAQ | 337 | PLNGAVHLYSQ | 378 |
| QLLPVQN | | QLGPVQN | | AQITPVQN | | AQTGRVQN | |
| ALNGTVHLYAQA | 724 | PLNGAVHLYAQA | 471 | PLNGAVHLYAQ | 227 | PLNGAVHLYAQ | 154 |
| QTGWVQN | | QRTSVQN | | AQVAKVQN | | AQVKQVQN | |
| QLNGAVHLYAQA | 585 | PLNGAVHLYAQA | 510 | PLNGAVHLYAQ | 336 | PLNGGVHLYAQ | 165 |
| QTGSVQN | | QSQPVQN | | AQTQMVQN | | AQTGRVQN | |
| ALNGAVHLYAQA | 485 | PLNGAVHLYAQA | 522 | PLNGAVHLYAQ | 253 | PLDGAVHLYAQ | 140 |
| QTGLVQN | | QVLGVQN | | AQTKAVQN | | PQTGWVQN | |
| PLNGAVHLYAQA | 517 | PLNGAVHLYAQA | 894 | PLNGAVHLYAQ | 778 | PLNGAVHLYAQ | 751 |
| QSTSVQN | | QSVSVQN | | AQSQQVQN | | AQTGWVLN | |
| PLNGAVHLYAQA | 442 | PLNGAVHLYAQA | 181 | PLNGAVHLYAQ | 191 | PLNGAVHLYAQ | 212 |
| QTSAVQN | | QTMQVQN | | AQISGVQN | | AQIQSVQN | |
| PINGAVHLYAQA | 588 | PLNGAVHLYAQA | 684 | PLNGAVHLYAQ | 296 | PLNGAVHLYAQ | 214 |
| QTGLVQN | | QSAVVQN | | AQVSRVQN | | AQLQRVQN | |
| PLNGAVHLYAQA | 175 | PLNGAVHLYAQA | 357 | PLNGAVHLYAQ | 502 | PLNGAVHLYAQ | 482 |
| QTTPVQN | | QLAGVQN | | AQTGWDQN | | AQTMMVQN | |
| PLNGSVHLYAQA | 660 | SLNGAVHLYAQA | 388 | ALNGAVHLYAQ | 241 | PLNGAVHLYAQ | 267 |
| QTGLVQN | | QTGRVQN | | AQTGRVQN | | AQVGNVQN | |
| PLNGAVHLYAQA | 641 | PLNGAVHLYAQA | 452 | PLNGAVHLYAQ | 358 | PLNGAVHLYAQ | 417 |
| QSALVQN | | QTGWVQT | | AQRTAVQN | | AQRIGVQN | |
| PVNGAVHLYAQA | 526 | PLNGAVHLYAQA | 290 | PLDGAVHVYAQ | 430 | PLNGAVHLYAQ | 309 |
| QTGLVQN | | QSGPVQN | | AQTGWVQN | | AQTGWVRN | |
| PLNGAVHLYAQA | 432 | PLNGAVHLYAQA | 288 | PLDGAVHLYSQ | 370 | PLNGAVHLYAQ | 161 |
| QLGSVQN | | QTNSVQN | | AQTGWVQN | | AQSAKVQN | |
| PLNGAVHLYAQA | 237 | PLNGAVHLYAQA | 543 | PLNGAVHLYAQ | 624 | PLNGAVHLYAQ | 173 |
| QVTPVQN | | QRLTVQN | | AQRMLVQN | | AQRIAVQN | |
| PLNGAVHLYAQA | 300 | ALNGAVHLYAQS | 1047 | PLNGAVHLYAQ | 929 | PLNGAVHLYAQ | 436 |
| QVSAVQN | | QTGWVQN | | AQSQMVQN | | AQVQNVQN | |
| PLNGAVHLYAQA | 852 | PLNGAVHLYAQA | 170 | PLNGAVHLYAQ | 196 | PLNGAVHLYAQ | 393 |
| QIGWVQN | | QTTQVQN | | AQVANVQN | | AQNVQVQN | |
| PLNGAVHLYAQA | 640 | PLNGAVHLYAQA | 469 | PLNGAVHLYAQ | 240 | PLNGAVHLYAQ | 456 |
| QTGFVQN | | QSAAVQN | | AQCTPVQN | | AQRIPVQN | |
| PLNGAVHLYAQA | 177 | PLNGAVHLYAQA | 262 | PLNGAVHLYAQ | 326 | PLNGAVHLYAQ | 305 |
| QLTPVQN | | QTSVVQN | | PQTGLVQN | | AQNAQVQN | |
| PLNGAVHLYAQA | 360 | PLNGAVHLYAQA | 332 | PLNGAVHLYAQ | 273 | PLNGAVHLYAQ | 480 |
| QTLAVQN | | QRLGVQN | | AQISSVQN | | AQTGWVQS | |
| QLNGAVHLYAQA | 719 | PLNGGVHLYAQA | 983 | PLNGAVHLYAQ | 220 | PLNGYVHLYAQ | 1009 |
| QTGGVQN | | QTGGVQN | | AQAGPVQN | | AQTGWVQN | |
| PLNGAVHLYAQA | 658 | PLNGAVHLYAQA | 635 | PLNGAVHLYAQ | 160 | PLNGAVHLYAQ | 841 |
| QLSAVQN | | QTGWVQK | | AQTAKVQN | | AQTGWVQY | |
| PLNGAVHLYAQA | 229 | PLNGAVHLYAQA | 174 | PLNGAVHLYAQ | 282 | PLNGAVHLYAQ | 392 |
| QTVGVQN | | QRASVQN | | AQRTGVQN | | AQNMQVQN | |
| PLNGAVHLYAQA | 256 | PLNGAVHLYAQA | 498 | PLNGAVHLYAQ | 258 | PLNGAVHLYAQ | 349 |
| QTAPVQN | | QSTQVQN | | AQLHPVQN | | AQTGWAQN | |
| PLNGAVHLYAQA | 318 | PLNGAVHLYAQA | 213 | PLNGAVHLYAQ | 276 | PLNGAVHLYAQ | 804 |
| QTMPVQN | | QCSPVQN | | AQTMTVQN | | AQTGWVEN | |
| PLNGAVHLYAQA | 362 | PLNGAVHLYAQA | 507 | PLNGAVHLYAQ | 561 | PLNGNVHLYAQ | 189 |
| QTSLVQN | | QSAGVQN | | AQAQPVQN | | AQTGGVQN | |
| PLNGAVHLYAQA | 343 | PLNGAVHLYAQA | 1038 | PLNGAVHLYAQ | 304 | PLDGAVHLYAQ | 150 |
| QVQSVQN | | QALPVQN | | AQQAPVQN | | AQTGGVQN | |
| PLNGAVHLYAQA | 783 | PLNGAVHLYAQA | 285 | PLNGAVHLYAQ | 188 | PLNGAVHLYAQ | 801 |
| QSSMVQN | | QVTSVQN | | AQRQPVQN | | AQTPSVQN | |
| PLNGAVHLYAQA | 1015 | PLNGAVHLYAQA | 346 | PLNGAVHLYAQ | 250 | PLNGAVHLYAQ | 458 |
| QSLPVQN | | QISPVQN | | AQLKSVQN | | AQTGWVQD | |
| PLNGAVHLYAQA | 184 | PLNGAVHLYAQA | 438 | PLNGAVHLYAQ | 568 | PLNGAVHLYAQ | 260 |
| QVSQVQN | | QSVGVQN | | AQNTTVQN | | AQLTNVQN | |
| PLNGAVHLYAQA | 277 | PLNGAVHLYAQA | 451 | RLDGAVHLYAQ | 602 | QLNGAVHLYAQ | 1032 |
| QRSSVQN | | QLTSVQN | | AQTGWVQN | | AQTGWVKN | |
| PLNGAVHLYAQA | 246 | PLNGAVHLYAQA | 222 | PLNGAVHLYAQ | 182 | PLNGAVHLYAQ | 218 |
| QTSTVQN | | QTTTVQN | | AQTSKVQN | | AQVTKVQN | |
| PLNGAVHLYAQA | 399 | PLNGAVHLYAQA | 312 | PLNGSVHLYAQ | 572 | QLNGAVHLYAQ | 735 |
| QVMSVQN | | QRSGVQN | | PQTGWVQN | | AQTGWVQT | |
| PLNGAVHLYAQA | 308 | PLNGAVHLYAQA | 490 | PLNGAVHLYAQ | 501 | ALNGAVHLYAQ | 1061 |
| QTTAVQN | | QTQLVQN | | AQRYSVQN | | AQTGWVQK | |
| PLNGSVHLYAQA | 294 | PLNGAVHLYAQA | 230 | PLNGAVHLYAQ | 861 | ALNGAVHLYAQ | 678 |
| QTGSVQN | | QLNPVQN | | AQKSSVQN | | AQTGWVQT | |
| PLNGAVHLYAQA | 293 | PLNGTVHLYAQA | 395 | PLNGAVHLYAQ | 323 | QLNGAVHLYAQ | 980 |
| QTQPVQN | | QTGGVQN | | AQNTPVQN | | AQTGWVQK | |
| PLNGAVHLYAQA | 468 | PLNGAVHLYAQA | 186 | PLNGAVHLYAQ | 352 | ALNGAVHLYAQ | 1081 |
| QLSTVQN | | QTVQVQN | | AQVGGVQN | | AQTGWVKN | |
| PLNGAVHLYAQA | 157 | PLNGAVHLYAQA | 202 | PLNGAVHLYAQ | 251 | QLNGAVHLYAQ | 961 |
| QLAPVQN | | QIASVQN | | AQIAAVQN | | AQTVSVQN | |
| PLNGAVHLYAQA | 166 | PLNGAVHLYAQA | 158 | PLNGAVHLYAQ | 317 | PLDSSVHLYAQ | 142 |
| QTVAVQN | | QLAQVQN | | AQLMAVQN | | AQTGWVQN | |
| PLNGAVHLYAQA | 363 | PLNGAVHLYAQA | 404 | PLNGAVHLYAQ | 407 | PLNGSVHLYAQ | 791 |
| QRLSVQN | | QLSRVQN | | AQMAPVQN | | AQTGWVKN | |
| PLNGAVHLYAQA | 862 | PLNGAVHLYAQA | 536 | PLNGAVHLYAQ | 271 | QLNGAVHLYAQ | 959 |
| QTGYVQN | | QLSNVQN | | AQVHPVQN | | AQTGWDQN | |
| PLNGAVHLYAQA | 425 | PLNGAVHLYAQA | 400 | PLNGAVHLYAQ | 301 | QLNGAVHLYAQ | 1113 |
| QTSGVQN | | QTTGVQN | | AQVLSVQN | | AQPGWVQN | |
| PLNGAVHLYAQA | 243 | PLNGAVHLYAQA | 913 | PLNGAVHLYAQ | 320 | PLNGAVHLYAQ | 559 |
| QTAGVQN | | QTSNVQN | | AQLANVQN | | AQLPPVQN | |
| PLNGAVHLYAQA | 616 | PLNGAVHLYAQA | 209 | PLNGAVHLYAQ | 601 | PLNGAVHLYAQ | 695 |
| QVMPVQN | | QSMAVQN | | AQATQVQN | | AQSPPVQN | |
| PLNGAVHLYAQA | 244 | PLNGAVHLYAQA | 437 | PLNGAVHLYAQ | 234 | PLNGSVHLYAQ | 748 |
| QTSQVQN | | QTKSVQN | | AQTNAVQN | | AQTGWVQK | |
| PLNGAVHLYAQA | 200 | PLNGAVHLYAQA | 465 | PLNGAVHLYAQ | 446 | PLNGGVHLYAQ | 864 |
| QTMAVQN | | QSVAVQN | | AQTANVQN | | AQTGWVKN | |
| PLNGAVHLYAQA | 372 | PLNGAVHLYAQA | 295 | PLNGAVHLYAQ | 255 | PLNGAVHLYAQ | 727 |
| QVGSVQN | | QTQQVQN | | AQVSNVQN | | AQTPAVQN | |
| PLNGAVHLYAQA | 564 | PLNGAVHLYAQA | 195 | PLNGAVHLYAQ | 683 | PVNGAVHLYAQ | 1051 |
| QTSWVQN | | QVQQVQN | | AQAAAVQN | | AQTGWVQT | |
| PLNGAVHLYAQA | 988 | PLNGAVHLYAQA | 537 | PLNGAVHLYAQ | 787 | PLNGSVHLYAQ | 366 |
| QTGVVQN | | QRLPVQN | | AQAVGVQN | | AQTGWVQT | |
| PLNGAVHLYAQA | 411 | PLNCAVHLYAQA | 975 | PLNGAVHLYAQ | 435 | PLNGGVHLYAQ | 906 |
| QTGTVQN | | QTGWVQN | | AQLTAVQN | | AQTGWVQK | |
| PLNGAVHLYAQA | 628 | PLNGAVHLYAQA | 420 | PLNGAVHLYAQ | 758 | PLNGAVHLYAQ | 992 |
| QTLGVQN | | QSAMVQN | | AQSANVQN | | AQTGCVQK | |
| PLNGAVHLYAQA | 257 | PLNGAVHLYAQA | 331 | PLNGAVHLYAQ | 525 | SLNGAVHLYAQ | 1095 |
| QLMPVQN | | QTAVVQN | | AQRAGVQN | | AQTGWVQH | |
| PLNGAVHLYAQA | 307 | PLNGAVHLYAQA | 574 | PLNGAVHLYAQ | 538 | | |
| QVQPVQN | | QTQWVQN | | AQRQGVQN | | | |
| PLNGAVHLYAQA | 535 | PLNGAVHLYAQA | 693 | PLNGAVHLYAQ | 544 | | |
| QTGMVQN | | QSSRVQN | | AQTLRVQN | | | |

TABLE 2
Exemplary Peptide

| | SEQ | | SEQ | |
|---|---|---|---|---|
| Pep- | ID | Amino Acid | ID | |
| tide | NO: | Sequence | NO: | Nucleotide Sequence |
| 1 | 3648 | PLNGAVHLY | 3660 | ccgcttaatggtgccgtccatct |
| | | | | ttat |
| 2 | 3649 | RDSPKGW | 3661 | cgtgattctccgaagggttggca |
| 3 | 3650 | YSTDVRM | 3662 | tattctacggatgtgaggatgca |
| 4 | 3651 | IVMNSLK | 3663 | attgttatgaattcgttgaaggc |
| 5 | 3652 | RESPRGL | 3664 | cgggagagtcctcgtgggctgca |
| 6 | 3653 | SFNDTRA | 3665 | agttttaatgatactagggctca |
| 7 | 3654 | GGTLAVVSL | 3666 | ggtggtacgttggccgtcgtgtc |
| | | | | gctt |
| 8 | 3655 | YGLPKGP | 3667 | tatgggttgccgaagggtcct |
| 9 | 3656 | STGTLRL | 3668 | tcgactgggacgcttcggctt |
| 10 | 3657 | YSTDERM | 3669 | tattcgacggatgagaggatg |
| 11 | 3658 | YSTDERK | 3670 | tattcgacggatgagaggaag |
| 12 | 3659 | YVSSVKM | 3671 | Tatgtttcgtctgttaagatg |
| 13 | 314 | PLNGAVHLYA | 6 | ccgcttaatggtgccgtccatct |
| | | QAQTGWVPN | | ttatGCTCAGGCGCAGACCGGCT |
| | | | | GGGTTccgAAC |
| 14 | 566 | PLNGAVHLYA | 9 | CCGCTGAATGGTGCGGTGCATCT |
| | | QAQLSPVKN | | GTATGCGCAGGCGCAGCTGTCTC |
| | | | | CGGTGAAGAAT |

In some embodiments, the peptide comprises an amino acid sequence comprising the following formula [N1]-[N2], wherein [N1] comprises X1, X2, X3, X4, and X5, and [N2] comprises the amino acid sequence of VHLY (SEQ ID NO: 4680), VHIY (SEQ ID NO: 4681), VHVY (SEQ ID NO: 4682), or VHHY (SEQ ID NO: 4683). In some embodiments, position X1 of [N1] is: P, Q, A, H, K, L, R, S, or T. In some embodiments, position X2 of [N1] is: L, I, V, H, or R. In some embodiments, position X3 of [N1] is: N, D, I, K, or Y. In some embodiments, position X4 of [N1] is: G, A, C, R, or S. In some embodiments, position X5 of [N1] is: A, 5, T, G, C, D, N, Q, V, or Y. In some embodiments [N1] comprises AL, PI, PL, QL, SL, TL, LN, LD, IN, NG, DG, DS, GA, SA, SS, GG, GN, GS, or GT. In some embodiments, [N1] comprises ALD, ALN, PIN, PLD, PLN, QLN, SLD, SLN, TLN, LNG, LDG, ING, LDS, NGA, DGA, DSA, DSS, NGG, NGN, NGS, NGT. In some embodiments, [N1] is or comprises SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGA (SEQ ID NO: 3679), PLNGG (SEQ ID NO: 4689), PLNGT (SEQ ID NO: 4690), PLDGA (SEQ ID NO: 469i), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722). In some embodiments, [N1] is or comprises ALDGA (SEQ ID NO: 4698), ALNGA (SEQ ID NO: 4686), PINGA (SEQ ID NO: 4697), PLDGA (SEQ ID NO: 4691), PLDSA (SEQ ID NO: 4701), PLDSS (SEQ ID NO: 4705), PLNGA (SEQ ID NO: 3679), PLNGG (SEQ ID NO: 4689), PLNGN (SEQ ID NO: 4693), PLNGS (SEQ ID NO: 4687), PLNGT (SEQ ID NO: 4690), QLNGA (SEQ ID NO: 4685), SLDGA (SEQ ID NO: 4694), SLNGA (SEQ ID NO: 4684), or TLNGA (SEQ ID NO: 4708). In some embodiments, wherein [N1]-[N2] comprises LDGAVHLY (SEQ ID NO: 4768), LNGAVHLY (SEQ ID NO: 4769), INGAVHLY (SEQ ID NO: 4770), LDSAVHLY (SEQ ID NO: 4771), LDSSVHLY (SEQ ID NO: 4772), LNGGVHLY (SEQ ID NO: 4773), LNGNVHLY (SEQ ID NO: 4774), LNGSVHLY (SEQ ID NO: 4775), LNGTVHLY (SEQ ID NO: 4776), LNGAVHIY (SEQ ID NO: 4777), LDGAVHVY (SEQ ID NO: 4778), LNGAVHHY (SEQ ID NO: 4779). In some embodiments, [N1]-[N2] is or comprises ALDGAVHLY (SEQ ID NO: 4780), ALNGAVHLY (SEQ ID NO: 4781), PINGAVHLY (SEQ ID NO: 4782), PLDGAVHLY (SEQ ID NO: 4783), PLDSAVHLY (SEQ ID NO: 4784), PLDSSVHLY (SEQ ID NO: 4785), PLNGAVHLY (SEQ ID NO: 3648), PLNGGVHLY (SEQ ID NO: 4786), PLNGNVHLY (SEQ ID NO: 4787), PLNGSVHLY (SEQ ID NO: 4788), PLNGTVHLY (SEQ ID NO: 4789), QLNGAVHLY (SEQ ID NO: 4790), SLDGAVHLY (SEQ ID NO: 4791), SLNGAVHLY (SEQ ID NO: 4792), TLNGAVHLY (SEQ ID NO: 4793), PLNGAVHIY (SEQ ID NO: 4794), PLDGAVHVY (SEQ ID NO: 4795), PLNGAVHHY (SEQ ID NO: 4796); an amino acid sequence comprising any portion of any of the aforesaid amino acid sequences (e.g., any 2, 3, 4, 5, 6, 7, or 8 amino acids, e.g., consecutive amino acids) thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, the peptide comprising the amino acid sequence comprising the formula of [N1]-[N2], further comprises [N3], which comprises X6, X7, X8, and X9. In some embodiments, position X6 of [N3] is: A, D, S, or T. In some embodiments, position X7 of [N3] is: Q, K, H, L, P, or R. In some embodiments, position X8 of [N3] is: A, P, E, or R. In some embodiments, position X9 of [N3] is Q, H, K, or P. In some embodiments, [N3] comprises AQ, SQ, AK, DQ, PQ, QA, QP, or KA. In some embodiments, [N3] comprises AQA, AQP, SQA, AKA, DQA, QAQ, QPQ, or KAQ. In some embodiments, [N3] is or comprises AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751). In some embodiments, [N3] is or comprises AQAQ (SEQ ID NO: 4737), AQPQ (SEQ ID NO: 4739), SQAQ (SEQ ID NO: 4738), AKAQ (SEQ ID NO: 4741), or DQAQ (SEQ ID NO: 4744). In some embodiments, is or comprises ALDGAVHLYAQAQ (SEQ ID NO: 4827), ALNGAVHLYAQAQ (SEQ ID NO: 4828), PINGAVHLYAQAQ (SEQ ID NO: 4829), PLDGAVHLYAQAQ (SEQ ID NO: 4830), PLDGAVHLYAQPQ (SEQ ID NO: 4831), PLDGAVHLYSQAQ (SEQ ID NO: 4832), PLDSAVHLYAQAQ (SEQ ID NO: 4833), PLDSSVHLYAQAQ (SEQ ID NO: 4834), PLNGAVHLYAKAQ (SEQ ID NO: 4835), PLNGAVHLYAQAQ (SEQ ID NO: 4836), PLNGAVHLYAQPQ (SEQ ID NO: 4837), PLNGAVHLYDQAQ (SEQ ID NO: 4838), PLNGAVHLYSQAQ (SEQ ID NO: 4839), PLNGGVHLYAQAQ (SEQ ID NO: 4840), PLNGNVHLYAQAQ (SEQ ID NO: 4841), PLNGSVHLYAQAQ (SEQ ID NO: 4842), PLNGTVHLYAQAQ (SEQ ID NO: 4843), QLNGAVHLYAQAQ (SEQ ID NO: 4844), SLDGAVHLYAQAQ (SEQ ID NO: 4845), SLNGAVHLYAQAQ (SEQ ID NO: 4846), TLNGAVHLYAQAQ (SEQ ID NO: 4847), PLNGAVHIYAQAQ (SEQ ID NO: 4848), PLDGAVHVYAQAQ (SEQ ID NO: 4849), PLNGAVHHYAQAQ (SEQ ID NO: 4850); an amino acid sequence comprising any portion of any of the aforesaid amino acid sequences (e.g., any 2, 3, 4, 5, 6, 7, 8, 9, 10, 11, or 12 amino acids, e.g., consecutive amino acids) thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, the peptide comprising the amino acid sequence comprising the formula [N1]-[N2], further comprises [N4], which comprises X10, X11, and X12. In some embodiments, position X10 of [N4] is: T, V, L, R, S, A, C, I, K, M, N, P, or Q. In some embodiments, position X1I of [N4] is: G, S, A, T, M, V, Q, L, H, I, K, N, P, R, or Y. In some embodiments, position X12 of [N4] is: W, S, P, G, A, Q, L, M, K, C, E, F, H, R, T, V, or Y. In some embodiments, [N4] comprises LS, TG, LA, LT, SA, SS, TL, TT, TS, TA, TV, VS, AA, AG, AS, AT, CS, CT, IA, IG, IL, IQ, IS, IT, LG, LH, LK, LM, LN, LQ, MA, NA, NM, NS, NT, NV, QA, RA, RG, RI, RL, RM, RN, RQ, RS, RT, RV, SG, SM, ST, SV, TK, TM, TN, TP, TQ, TR, VA, VG, VH, VK, VL, VM, VN, VQ, VR, VT, PG, LV, SP, GW, AP, GR, AL, AW, GG, GS, GP, QP, QS, AH, AN, AQ, AR, GQ, HP, KS, MG, MP, MQ, MS, NP, QQ, QR, SH, SK, SQ, SR, IP, VE, AK, AM, AV, GA, GC, GT, KA, KP, KQ, LP, MK, MN, MT, NQ, PP, QH, QK, QM, QN, QT, RW, SL, VW, GK, GN, NG, RP, SN, GL, or VP. In some embodiments, [N4] is or comprises TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW.

In some embodiments, the peptide comprising the amino acid sequence comprising the formula [N1]-[N2], further comprises [N5] which comprises X13, X14, and X15. In some embodiments, position X13 of [N5] is: V, D, F, G, L, A, E, or I. In some embodiments, position X14 of [N5] is: Q, K, R, H, E, L, or P. In some embodiments, position X15 of [N5] is: N, T, K, H, D, Y, S, I, or P. In some embodiments, [N5] comprises VQ, AQ, DQ, FQ, VL, LQ, EQ, GQ, VP, VR, VK, QN, QS, QT, QK, QH, LN, QI, PN, QD, QP, RN, or KN. In some embodiments, [N5] is or comprises VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH. In some embodiments [N5] is or comprises VQN, AQN, VQS, DQN, VQT, VQK, VQH, FQN, VLN, LQN, VQI, EQN, GQT, VPN, VQD, VQP, VRN, or VKN. In some embodiments [N4]-[N5] is or comprises TGWVQN (SEQ ID NO: 4851), LAAVQN (SEQ ID NO: 4852), LTPVQN (SEQ ID NO: 4853), SAPVQN (SEQ ID NO: 4854), SSPVQN (SEQ ID NO: 4855), TGRVQN (SEQ ID NO: 4856), TGWAQN (SEQ ID NO: 4857), TGWVQS (SEQ ID NO: 4858), TLAVQN (SEQ ID NO: 4859), TTSVQN (SEQ ID NO: 4860), TSPVQN (SEQ ID NO: 4861), TALVQN (SEQ ID NO: 4862), TAWVQN (SEQ ID NO: 4863), TGGVQN (SEQ ID NO: 4864), TGSVQN (SEQ ID NO: 4865), TGWDQN (SEQ ID NO: 4866), TVSVQN (SEQ ID NO: 4867), VSPVQN (SEQ ID NO: 4868), VSSVQN (SEQ ID NO: 4869), AAPVQN (SEQ ID NO: 4870), AGPVQN (SEQ ID NO: 4871), ASPVQN (SEQ ID NO: 4872), ATPVQN (SEQ ID NO: 4873), CSPVQN (SEQ ID NO: 4874), CTPVQN (SEQ ID NO: 4875), IAAVQN (SEQ ID NO: 4876), IAGVQN (SEQ ID NO: 4877), IASVQN (SEQ ID NO: 4878), IGGVQN (SEQ ID NO: 4879), IGSVQN (SEQ ID NO: 4880), ILGVQN (SEQ ID NO: 4881), IQPVQN (SEQ ID NO: 4882), IQSVQN (SEQ ID NO: 4883), ISGVQN (SEQ ID NO: 4884), ISPVQN (SEQ ID NO: 4885), ISSVQN (SEQ ID NO: 4886), ITPVQN (SEQ ID NO: 4887), LAGVQN (SEQ ID NO: 4888), LAHVQN (SEQ ID NO: 4889), LANVQN (SEQ ID NO: 4890), LAPVQN (SEQ ID NO: 4891), LAPVQT (SEQ ID NO: 4892), LAQVQN (SEQ ID NO: 4893), LARVQN (SEQ ID NO: 4894), LASVQN (SEQ ID NO: 4895), LATVQN (SEQ ID NO: 4896), LGPVQN (SEQ ID NO: 4897), LGQVQN (SEQ ID NO: 4898), LGSVQN (SEQ ID NO: 4899), LHPVQN (SEQ ID NO: 4900), LKSVQN (SEQ ID NO: 4901), LMAVQN (SEQ ID NO: 4902), LMGVQN (SEQ ID NO: 4903), LMPVQN (SEQ ID NO: 4904), LMQVQN (SEQ ID NO: 4905), LMSVQN (SEQ ID NO: 4906), LNPVQN (SEQ ID NO: 4907), LQPVQN (SEQ ID NO: 4908), LQQVQN (SEQ ID NO: 4909), LQRVQN (SEQ ID NO: 4910), LSHVQN (SEQ ID NO: 4911), LSKVQN (SEQ ID NO: 4912), LSPVQK (SEQ ID NO: 4913), LSPVQN (SEQ ID NO: 4914), LSQVQN (SEQ ID NO: 4915), LSRVQN (SEQ ID NO: 4916), LSTVQN (SEQ ID NO: 4917), LTAVQN (SEQ ID NO: 4918), LTNVQN (SEQ ID NO: 4919), LTSVQN (SEQ ID NO: 4920), MAPVQN (SEQ ID NO: 4921), NAQVQN (SEQ ID NO: 4922), NASVQN (SEQ ID NO: 4923), NMQVQN (SEQ ID NO: 4924), NSPVQN (SEQ ID NO: 4925), NTPVQN (SEQ ID NO: 4926), NVQVQN (SEQ ID NO: 4927), QAPVQN (SEQ ID NO: 4928), RAAVQN (SEQ ID NO: 4929), RAQVQN (SEQ ID NO: 4930), RASVQN (SEQ ID NO: 4931), RGGVQN (SEQ ID NO: 4932), RGSVQN (SEQ ID NO: 4933), RIAVQN (SEQ ID NO: 4934), RIGVQN (SEQ ID NO: 4935), RIPVQN (SEQ ID NO: 4936), RLGVQN (SEQ ID NO: 4937), RLSVQN (SEQ ID NO: 4938), RMSVQN (SEQ ID NO: 4939), RNSVQN (SEQ ID NO: 4940), RQPVQN (SEQ ID NO: 4941), RSAVQN (SEQ ID NO: 4942), RSGVQN (SEQ ID NO: 4943), RSPVQN (SEQ ID NO: 4944), RSQVQN (SEQ ID NO: 4945), RSSVQN (SEQ ID NO: 4946), RSTVQN (SEQ ID NO: 4947), RTAVQN (SEQ ID NO: 4948), RTGVQN (SEQ ID NO: 4949), RTLVQN (SEQ ID NO: 4950), RTSVQN (SEQ ID NO: 4951), RTTVQN (SEQ ID NO: 4952), RVEVQN (SEQ ID NO: 4953), SAAVQN (SEQ ID NO: 4954), SAKVQN (SEQ ID NO: 4955), SAMVQN (SEQ ID NO: 4956), SAQVQN (SEQ ID NO: 4957), SGPVQN (SEQ ID NO: 4958), SMAVQN (SEQ ID NO: 4959), SMGVQN (SEQ ID NO: 4960), SMQVQN (SEQ ID NO: 4961), SMSVQN (SEQ ID NO: 4962), STPVQN (SEQ ID NO: 4963), SVAVQN (SEQ ID NO: 4964), SVGVQN (SEQ ID NO: 4965), TAAVQN (SEQ ID NO: 4966), TAGVQN (SEQ ID NO: 4967), TAKVQN (SEQ ID NO: 4968), TAMVQN (SEQ ID NO: 4969), TANVQN (SEQ ID NO: 4970), TAPVQN (SEQ ID NO: 4971), TAPVQT (SEQ ID NO: 4972), TAQVQN (SEQ ID NO: 4973), TASVQN (SEQ ID NO: 4974), TASVQT (SEQ ID NO: 4975), TATVQN (SEQ ID NO: 4976), TAVVQN (SEQ ID NO: 4977), TAWDQN (SEQ ID NO: 4978), TAWVQH (SEQ ID NO: 4979), TAWVQT (SEQ ID NO: 4980), TGAVQN (SEQ ID NO: 4981), TGCFQN (SEQ ID NO: 4982), TGGAQN (SEQ ID NO: 4983), TGGFQN (SEQ ID NO: 4984), TGGVLN (SEQ ID NO: 4985), TGGVQH (SEQ ID NO: 4986), TGGVQK (SEQ ID NO: 4987), TGGVQT (SEQ ID NO: 4988), TGPVQN (SEQ ID NO: 4989), TGSAQN (SEQ ID NO: 4990), TGSLQN (SEQ ID NO: 4991), TGSVQH (SEQ ID NO: 4992), TGSVQI (SEQ ID NO: 4993), TGSVQS (SEQ ID NO: 4994), TGSVQT (SEQ ID NO: 4995), TGTVQN (SEQ ID NO: 4996), TGWEQN (SEQ ID NO: 4997), TGWFQN (SEQ ID NO: 4998), TGWGQT (SEQ ID NO: 4999), TGWVPN (SEQ ID NO: 5000), TGWVQD (SEQ ID NO: 5001), TGWVQP (SEQ ID NO: 5002), TGWVQT (SEQ ID NO: 5003), TGWVRN (SEQ ID NO: 5004), TKAVQN (SEQ ID NO: 5005), TKPVQN (SEQ ID NO: 5006), TKQVQN (SEQ ID NO: 5007), TKSVQN (SEQ ID NO: 5008), TLPVQN (SEQ ID NO: 5009), TLQVQN (SEQ ID NO: 5010), TMAVQN (SEQ ID NO: 5011), TMGVQN (SEQ ID NO: 5012), TMKVQN (SEQ ID NO: 5013), TMNVQN (SEQ ID NO: 5014), TMPVQN (SEQ ID NO: 5015), TMQVQN (SEQ ID NO: 5016), TMSVKN (SEQ ID NO: 5017), TMSVQN (SEQ ID NO: 5018), TMSVQT (SEQ ID NO: 5019), TMTVQN (SEQ ID NO: 5020), TNAVQN (SEQ ID NO: 5021), TNQVQN (SEQ ID NO: 5022), TNSVQN (SEQ ID NO: 5023), TPPVQN (SEQ ID NO: 5024), TQHVQN (SEQ ID NO: 5025), TQKVQN (SEQ ID NO: 5026), TQMVQN (SEQ ID NO: 5027), TQNVQN (SEQ ID NO: 5028), TQPVQN (SEQ ID NO: 5029), TQQVQN (SEQ ID NO: 5030), TQTVQN (SEQ ID NO: 5031), TRWDQN (SEQ ID NO: 5032), TSAVQN (SEQ ID NO: 5033), TSGVQN (SEQ ID NO: 5034), TSHVQN (SEQ ID NO: 5035), TSKVQN (SEQ ID NO: 5036), TSLVQN (SEQ ID NO: 5037), TSMVQN (SEQ ID NO: 5038), TSPDQN (SEQ ID NO: 5039), TSQVQN (SEQ ID NO: 5040), TSSVQN (SEQ ID NO: 5041), TSSVQT (SEQ ID NO: 5042), TSTVQN (SEQ ID NO: 5043), TSVVQN (SEQ ID NO: 5044), TTAVQN (SEQ ID NO: 5045), TTGVQN (SEQ ID NO: 5046), TTKVQN (SEQ ID NO: 5047), TTPVQN (SEQ ID NO: 5048), TTPVQT (SEQ ID NO: 5049), TTQVQN (SEQ ID NO: 5050), TTTVQN (SEQ ID NO: 5051), TVAVQN (SEQ ID NO: 5052), TVAVQT (SEQ ID NO: 5053), TVGVQN (SEQ ID NO: 5054), TVQVQN (SEQ ID NO: 5055), TVSVKN (SEQ ID NO: 5056), TVWVQK (SEQ ID NO: 5057), VAAVQN (SEQ ID NO: 5058), VAGVQN (SEQ ID NO: 5059), VAKVQN (SEQ ID NO: 5060), VANVQN (SEQ ID NO: 5061), VAQVQN (SEQ ID NO: 5062), VASVQN (SEQ ID NO: 5063), VATVQN (SEQ ID NO: 5064), VGGVQN (SEQ ID NO: 5065), VGKVQN (SEQ ID NO: 5066), VGNVQN (SEQ ID NO: 5067), VGSVQN (SEQ ID NO: 5068), VHPVQN (SEQ ID NO: 5069), VKAVQN (SEQ ID NO: 5070), VKPVQN (SEQ ID NO: 5071), VKQVQN (SEQ ID NO: 5072), VLPVQN (SEQ ID NO: 5073), VLSVQN (SEQ ID NO: 5074), VMAVQN (SEQ ID NO: 5075), VMQVQN (SEQ ID NO: 5076), VMSVQN (SEQ ID NO: 5077), VNAVQN (SEQ ID NO: 5078), VNGVQN (SEQ ID NO: 5079), VNSVQN (SEQ ID NO: 5080), VQAVQN (SEQ ID NO: 5081), VQNVQN (SEQ ID NO: 5082), VQPVQN (SEQ ID NO: 5083), VQQVQN (SEQ ID NO: 5084), VQSVQN (SEQ ID NO: 5085), VQTVQN (SEQ ID NO: 5086), VRPVQN (SEQ ID NO: 5087), VSAVQN (SEQ ID NO: 5088), VSGVQN (SEQ ID NO: 5089), VSNVQN (SEQ ID NO: 5090), VSPVQT (SEQ ID NO: 5091), VSQVQN (SEQ ID NO: 5092), VSRVQN (SEQ ID NO: 5093), VSSVQK (SEQ ID NO: 5094), VSSVQT (SEQ ID NO: 5095), VSTVQN (SEQ ID NO: 5096), VTAVQN (SEQ ID NO: 5097), VTGVQN (SEQ ID NO: 5098), VTKVQN (SEQ ID NO: 5099), VTPVQN (SEQ ID NO: 5100), VTSVQN (SEQ ID NO: 5101), TGLVQN (SEQ ID NO: 5102), TGWVKN (SEQ ID NO: 5103), PGWVQN (SEQ ID NO: 5104), TGWVQH (SEQ ID NO: 5105), LSGVQN (SEQ ID NO: 5106), LSSVQN (SEQ ID NO: 5107), LVPVQN (SEQ ID NO: 5108); an amino acid sequence comprising any portion of any of the aforesaid amino acid sequences (e.g., any 2, 3, 4, 5, or 6 amino acids, e.g., consecutive amino acids) thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, [N1]-[N2]-[N3]-[N4]-[N5] comprises the amino acid sequence of any of SEQ ID NOs: 139-1138; an amino acid sequence comprising at least one, two, or three but no more than four modifications, e.g., substitutions (e.g., conservative substitutions), insertions, or deletions, relative to any of SEQ ID NOs: 139-1138; an amino acid sequence comprising at least one, two, or three but no more than four different amino acids, relative to any one of SEQ ID NOs: 139-1138. In some embodiments, [N1]-[N2]-[N3]-[N4]-[N5] comprises the amino acid sequence of any of SEQ ID NOs: 139-476; an amino acid sequence comprising at least one, two, or three but no more than four modifications, e.g., substitutions (e.g., conservative substitutions), insertions, or deletions, relative to any of SEQ ID NOs: 139-476; an amino acid sequence comprising at least one, two, or three but no more than four different amino acids, relative to any one of SEQ ID NOs: 139-476.

In some embodiments, [N2] is present immediately subsequent to [N1]. In some embodiments, the peptide comprises from N to C-terminus, [N1]-[N2]. In some embodiments, the peptide comprises from N to C-terminus, [N1]-[N2]-[N3]. In some embodiments, the peptide comprises from N to C-terminus, [N1]-[N2]-[N3]-[N4]. In some embodiments, the peptide comprises from N to C-terminus, [N1]-[N2]-[N3]-[N4]-[N5].

In some embodiments, the peptide comprises an amino acid sequence having the formula [A][B], wherein [A] comprises the amino acid sequence of PLNGA (SEQ ID NO: 3679), and [B] comprises X1, X2, X3, and X4. In some embodiments, position X1 of [B] is: V, I, L, A, F, D, or G. In some embodiments, position X2 of [B] is H, N, Q, P, D, L, R, or Y. In some embodiments, position X3 of [B] is L, H, I, R, or V. In some embodiments, position X4 of [B] is Y. In some embodiments, [B] comprises VH, VN, VQ, IH, LH, VP, VD, AH, FH, DH, VL, GH, VR, VY, LY, HY, IY, RY, HL, HH, HI, NL, QL, PL, DL, HR, LL, RL, HV, or YL. In some embodiments, B comprises VHL, VHH, VHI, VNL, VQL, IHL, LHL, VPL, VDL, AHL, VHR, FHL, DHL, VLL, GHL, VRL, VHV, VYL, HLY, HHY, HIY, NLY, QLY, PLY, DLY, HRY, LLY, RLY, HVY, or YLY. In some embodiments, [B] is or comprises VHLY (SEQ ID NO: 4680), VHHY (SEQ ID NO: 4683), VHIY (SEQ ID NO: 4681), VNLY (SEQ ID NO: 4724), VQLY (SEQ ID NO: 4729), IHLY (SEQ ID NO: 4730), LHLY (SEQ ID NO: 4727), VPLY (SEQ ID NO: 4723), VDLY (SEQ ID NO: 4731), AHLY (SEQ ID NO: 4732), VHRY (SEQ ID NO: 4725), FHLY (SEQ ID NO: 4726), DHLY (SEQ ID NO: 4728), VLLY (SEQ ID NO: 4733), GHLY (SEQ ID NO: 4734), VRLY (SEQ ID NO: 4735), VHVY (SEQ ID NO: 4682), or VYLY (SEQ ID NO: 4736). In some embodiments, [B] is or comprises VHLY (SEQ ID NO: 4680), VHHY (SEQ ID NO: 4683), or VHIY (SEQ ID NO: 4681). In some embodiments, [A][B] is or comprises PLNGAVHLY (SEQ ID NO: 3648), PLNGAVHHY (SEQ ID NO: 4796), PLNGAVHIY (SEQ ID NO: 4794), PLNGAVNLY (SEQ ID NO: 5123), PLNGAVQLY (SEQ ID NO: 5124), PLNGAIHLY (SEQ ID NO: 5125), PLNGALHLY (SEQ ID NO: 5126), PLNGAVPLY (SEQ ID NO: 5127), PLNGAVDLY (SEQ ID NO: 5128), PLNGAAHLY (SEQ ID NO: 5129), PLNGAVHRY (SEQ ID NO: 5130), PLNGAFHLY (SEQ ID NO: 5131), PLNGADHLY (SEQ ID NO: 5132), PLNGAVLLY (SEQ ID NO: 5133), PLNGAGHLY (SEQ ID NO: 5134), PLNGAVRLY (SEQ ID NO: 5135), PLNGAVHVY (SEQ ID NO: 5136), or PLNGAVYLY (SEQ ID NO: 5137); an amino acid sequence comprising any portion of any of the aforesaid amino acid sequences, e.g., any 2, 3, 4, 5, 6, 7, or 8 amino acids, e.g., consecutive amino acids, thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the amino aforesaid acid sequences.

In some embodiments, a peptide comprising an amino acid sequence comprising the formula [A][B], further comprises [C] which comprises X4, X5, X6, and X7. In some embodiments, position X4 of [C] is: A, D, S, or T. In some embodiments, position X5 of [C] is Q, K, H, L, P, or R. In some embodiments, position X6 of [C] is A, P, or E. In some embodiments, position X7 of [C] is Q, H, K, or P. In some embodiments [C] comprises AQ, AK, DQ, SQ, AH, AL, AP, AR, TQ, PQ, EQ, QA, QP, KA, HA, QE, LA, PA, or RA. In some embodiments [C] comprises AQA, AQP, AKA, DQA, SQA, AHA, AQE, ALA, APA, ARA, TQA, QAQ, QPQ, KAQ, HAQ, QEQ, QAK, LAQ, PAQ, RAQ, QAH, or QAP. In some embodiments [C] is or comprises AQAQ (SEQ ID NO: 4737), AQPQ (SEQ ID NO: 4739), AKAQ (SEQ ID NO: 4741), DQAQ (SEQ ID NO: 4744), SQAQ (SEQ ID NO: 4738), AHAQ (SEQ ID NO: 4742), AQEQ (SEQ ID NO: 4748), AQAK (SEQ ID NO: 4746), ALAQ (SEQ ID NO: 4749), APAQ (SEQ ID NO: 4745), ARAQ (SEQ ID NO: 4750), AQAH (SEQ ID NO: 4747), AQAP (SEQ ID NO: 4743), or TQAQ (SEQ ID NO: 4751). In some embodiments, [C] is or comprises AQAQ (SEQ ID NO: 4737), AQPQ (SEQ ID NO: 4739), AKAQ (SEQ ID NO: 4741), DQAQ (SEQ ID NO: 4744), or SQAQ (SEQ ID NO: 4738). In some embodiments [B][C] is or comprises VHLYAQAQ (SEQ ID NO: 4797), VHHYAQAQ (SEQ ID NO: 4804), VHLYAQPQ (SEQ ID NO: 4798), VHLYAKAQ (SEQ ID NO: 4800), VHLYDQAQ (SEQ ID NO: 4801), VHLYSQAQ (SEQ ID NO: 4799), VHIYAQAQ (SEQ ID NO: 4802), VHLYAHAQ (SEQ ID NO: 5138), VNLYAQAQ (SEQ ID NO: 5139), VQLYAQAQ (SEQ ID NO: 5140), VHLYAQEQ (SEQ ID NO: 5141), IHLYAQAQ (SEQ ID NO: 5142), LHLYAQAQ (SEQ ID NO: 5143), VPLYAQAQ (SEQ ID NO: 5144), VHLYAQAK (SEQ ID NO: 5145), VDLYAQAQ (SEQ ID NO: 5146), AHLYAQAQ (SEQ ID NO: 5147), VHRYAQAQ (SEQ ID NO: 5148), FHLYAQAQ (SEQ ID NO: 5149), VHLYALAQ (SEQ ID NO: 5150), DHLYAQAQ (SEQ ID NO: 5151), VHLYAPAQ (SEQ ID NO: 5152), VHLYARAQ (SEQ ID NO: 5153), VHLYAQAH (SEQ ID NO: 5154), VLLYAQAQ (SEQ ID NO: 5155), VHLYAQAP (SEQ ID NO: 5156), GHLYAQAQ (SEQ ID NO: 5157), VRLYAQAQ (SEQ ID NO: 5158), VHVYAQAQ (SEQ ID NO: 4803), VYLYAQAQ (SEQ ID NO: 5159), VHLYTQAQ (SEQ ID NO: 5160); an amino acid sequence comprising any portion of any of the aforesaid amino acid sequences, e.g., any 2, 3, 4, 5, 6, or 7 amino acids, e.g., consecutive amino acids, thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences. In some embodiments, [A][B][C][D] is or comprises PLNGAVHLYAQAQ (SEQ ID NO: 4836), PLNGAVHHYAQAQ (SEQ ID NO: 4850), PLNGAVHLYAQPQ (SEQ ID NO: 4837), PLNGAVHLYAKAQ (SEQ ID NO: 4835), PLNGAVHLYDQAQ (SEQ ID NO: 4838), PLNGAVHLYSQAQ (SEQ ID NO: 4839), PLNGAVHIYAQAQ (SEQ ID NO: 4848), PLNGAVHLYAHAQ (SEQ ID NO: 5181), PLNGAVNLYAQAQ (SEQ ID NO: 5182), PLNGAVQLYAQAQ (SEQ ID NO: 5183), PLNGAVHLYAQEQ (SEQ ID NO: 5184), PLNGAIHLYAQAQ (SEQ ID NO: 5185), PLNGALHLYAQAQ (SEQ ID NO: 5186), PLNGAVPLYAQAQ (SEQ ID NO: 5187), PLNGAVHLYAQAK (SEQ ID NO: 5188), PLNGAVDLYAQAQ (SEQ ID NO: 5189), PLNGAAHLYAQAQ (SEQ ID NO: 5190), PLNGAVHRYAQAQ (SEQ ID NO: 5191), PLNGAFHLYAQAQ (SEQ ID NO: 5192), PLNGAVHLYALAQ (SEQ ID NO: 5193), PLNGADHLYAQAQ (SEQ ID NO: 5194), PLNGAVHLYAPAQ (SEQ ID NO: 5195), PLNGAVHLYARAQ (SEQ ID NO: 5196), PLNGAVHLYAQAH (SEQ ID NO: 5197), PLNGAVLLYAQAQ (SEQ ID NO: 5198), PLNGAVHLYAQAP (SEQ ID NO: 5199), PLNGAGHLYAQAQ (SEQ ID NO: 5200), PLNGAVRLYAQAQ (SEQ ID NO: 5201), PLNGAVHVYAQAQ (SEQ ID NO: 5202), PLNGAVYLYAQAQ (SEQ ID NO: 5203), PLNGAVHLYTQAQ (SEQ ID NO: 5204); an amino acid sequence comprising any portion of any of the aforesaid amino acid sequences, e.g., any 2, 3, 4, 5, 6, 7, 8, 9, 10, 11, or 12 amino acids, e.g., consecutive amino acids, thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, the peptide comprising an amino acid sequence comprising the formula [A][B], further comprises [D], which comprises X8, X9, and X10. In some embodiments, position X8 of [D] is: T, V, S, L, R, I, A, N, C, Q, M, P, or K. In some embodiments, position X9 of [D] is: T, M, A, G, K, S, Q, V, I, R, N, P, L, H, or Y. In some embodiments, position X10 of [D] is: K, Q, W, S, P, C, A, G, N, T, R, V, M, H, L, E, F, or Y. In some embodiments, [D] comprises TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, PG, QS, RY, SQ, NQ, LL, LP, AQ, TY, NL, SP, LV, KG, VP, AV, KS, AM, SL, AL, RP, IP, MK, AW, GS, KQ, AP, SK, AK, GC, QK, MQ, QP, GP, QQ, AN, GK, QR, PP, AR, GG, MS, NP, KP, MN, KA, SN, MP, HP, GN, RW, MT, SR, GW, QH, GL, QM, VW, MG, AH, QT, GR, SH, GQ, GT, GA, NG, QN, VE, MM, QL, QG, YS, GM, LR, AF, PQ, SW, QW, YA, ML, GF, PA, PS, PT, GY, GV, PW, PR. In some embodiments, [D] is or comprises TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG.

In some embodiments, the peptide comprising the amino acid sequence comprising the formula [A][B], further comprises [E], which comprises X11, X12, and X13. In some embodiments, X1I of [E] is: V, D, F, A, E, L, G, or I. In some embodiments, X12 of [E] is Q, R, P, K, L, H, or E. In some embodiments, X13 of [E] is: N, H, S, T, P, K, I, D, or Y. In some embodiments, [E] comprises VQ, DQ, FQ, VR, VP, VK, AQ, EQ, LQ, GQ, VL, VH, VE, DK, GH, IQ, QN, QH, QS, QT, QP, RN, PN, KN, QK, QI, LN, QD, HN, KT, KK, EN, QY, or PH. In some embodiments, [E] is or comprises VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH.

In some embodiments, [A][B][C][D][E] comprises the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, or 1135; an amino acid sequence comprising any portion of any of the aforesaid amino acid sequences; an amino acid sequence comprising at least one, two, or three but no more than four modifications, e.g., substitutions (e.g., conservative substitutions), insertions, or deletions, relative to any of the aforesaid amino acid sequences; an amino acid sequence comprising at least one, two, or three but no more than four different amino acids, relative to any one of the aforesaid amino acid sequences.

In some embodiments, [B] is present immediately subsequent to [A]. In some embodiments, the peptide comprises from N-terminus to C-terminus [A][B]. In some embodiments, the peptide comprises from N-terminus to C-terminus [A][B][C]. In some embodiments, the peptide comprises from N-terminus to C-terminus [A][B][C][D]. In some embodiments, the peptide comprises from N-terminus to C-terminus [A][B][C][D][E].

In some embodiments, the peptide comprises at least 3, 4, 5, 6, 7, 8, 9, 10, 11, 12, 13, 14, 15, 16, 17, or 18 consecutive amino acids from any one of SEQ ID NOs: 139-1138.

In some embodiments, the peptide comprises at least 3, 4, 5, 6, 7, 8, 9, 10, 11, 12, 13, 14, 15, 16, 17, or 18 consecutive amino acids from any one of SEQ ID NOs: 139-476.

In some embodiments, the peptide comprises at least 3, 4, 5, 6, 7, 8, 9, 10, 11, 12, 13, 14, 15, 16, 17, or 18 consecutive amino acids from any one of the amino acid sequences provided in Table 1B.

In some embodiments, the peptide comprises at least 3, 4, 5, 6, 7, 8, 9, 10, 11, 12, 13, 14, 15, 16, 17, or 18 consecutive amino acids from any one of the amino acid sequences provided in Table 11.

In some embodiments, the peptide comprises at least 3, 4, 5, 6, 7, 8, 9, 10, 11, 12, 13, 14, 15, 16, 17, or 18 consecutive amino acids from any one of the amino acid sequences provided in Table 20.

In some embodiments, the peptide comprises at least 3, 4, 5, 6, 7, 8, or 9 consecutive amino acids from the amino acid sequence of any of SEQ ID NO: 1725-3622. In some embodiments, the peptide comprises at least 3, 4, 5, 6, 7, 8, or 9 consecutive amino acids from the amino acid sequence of any of SEQ ID NO: 3648-3659.

In some embodiments, the 3 consecutive amino acids comprise PLN. In some embodiments, the 4 consecutive amino acids comprise PLNG (SEQ ID NO: 3678). In some embodiments, the 5 consecutive amino acids comprise PLNGA (SEQ ID NO: 3679). In some embodiments, the 6 consecutive amino acids comprise PLNGAV (SEQ ID NO: 3680). In some embodiments, the 7 consecutive amino acids comprise PLNGAVH (SEQ ID NO: 3681). In some embodiments, the 8 consecutive amino acids comprise PLNGAVHL (SEQ ID NO: 3682). In some embodiments, the 9 consecutive amino acids comprise PLNGAVHLY (SEQ ID NO: 3648).

In some embodiments, the four consecutive amino acids comprise NGAV (SEQ ID NO: 3683). In some embodiments, the four consecutive amino acids comprise GAVH (SEQ ID NO: 3684). In some embodiments, the five consecutive amino acids comprise NGAVH (SEQ ID NO: 3685). In some embodiments, the five consecutive amino acids comprise GAVHL (SEQ ID NO: 3686). In some embodiments, the five consecutive amino acids comprise AVHLY (SEQ ID NO: 3687). In some embodiments, the six consecutive amino acids comprise NGAVHL (SEQ ID NO: 3688). In some embodiments, the seven consecutive amino acids comprise NGAVHLY (SEQ ID NO: 3689).

In some embodiments, the 3 consecutive amino acids comprise YST. In some embodiments, the 4 consecutive amino acids comprise YSTD (SEQ ID NO: 3690). In some embodiments, the 5 consecutive amino acids comprise YSTDE (SEQ ID NO: 3691). In some embodiments, the 5 consecutive amino acids comprise YSTDV (SEQ ID NO: 3700). In some embodiments, the 6 consecutive amino acids comprise YSTDER (SEQ ID NO: 3692). In some embodiments, the 6 consecutive amino acids comprise YSTDVR (SEQ ID NO: 3701). In some embodiments, the 7 consecutive amino acids comprise YSTDERM (SEQ ID NO: 3657). In some embodiments, the 7 consecutive amino acids comprise YSTDERK (SEQ ID NO: 3658). In some embodiments, the 7 consecutive amino acids comprise YSTDVRM (SEQ ID NO: 3650).

In some embodiments, the 3 consecutive amino acids comprise IVM. In some embodiments, the 4 consecutive amino acids comprise IVMN (SEQ ID NO: 3693). In some embodiments, the 5 consecutive amino acids comprise IVMNS (SEQ ID NO: 3694). In some embodiments, the 6 consecutive amino acids comprise IVMNSL (SEQ ID NO: 3695). In some embodiments, the 7 consecutive amino acids comprise IVMNSLK (SEQ ID NO: 3651).

In some embodiments, the peptide comprises an amino sequence comprising at least one, two, or three but no more than four modifications, e.g., substitutions (e.g., conservative substitutions), insertions, or deletions, relative to the amino acid sequence of any one of SEQ ID NOs: 139-1138. In some embodiments, the peptide comprises an amino sequence comprising at least one, two, or three but no more than four different amino acids, relative to the amino acid sequence of any one of SEQ ID NOs: 139-1138.

In some embodiments, the peptide comprises an amino sequence comprising at least one, two, or three but no more than four modifications, e.g., substitutions (e.g., conservative substitutions), insertions, or deletions, relative to the amino acid sequence of any one of SEQ ID NOs: 139-476. In some embodiments, the peptide comprises an amino sequence comprising at least one, two, or three but no more than four different amino acids, relative to the amino acid sequence of any one of SEQ ID NOs: 139-476.

In some embodiments, the peptide comprises an amino sequence comprising at least one, two, or three but no more than four modifications, e.g., substitutions (e.g., conservative substitutions), insertions, or deletions, relative to any one of the amino acid sequences in Table 1B. In some embodiments, the peptide comprises an amino sequence comprising at least one, two, or three but no more than four different amino acids, relative to any one of the amino acid sequences in Table 1B.

In some embodiments, the peptide comprises an amino sequence comprising at least one, two, or three but no more than four modifications, e.g., substitutions (e.g., conservative substitutions), insertions, or deletions, relative to the amino acid sequence of any one of SEQ ID NOs: 1139-1172. In some embodiments, the peptide comprises an amino sequence comprising at least one, two, or three but no more than four different amino acids, relative to the amino acid sequence of any one of SEQ ID NOs: 1139-1172.

In some embodiments, the peptide comprises an amino acid sequence comprising at least one, two, or three but no more than four modifications, e.g., substitutions (e.g., conservative substitutions), insertions, or deletions, relative to the amino acid sequence of any of SEQ ID NO: 1725-3622. In some embodiments, the peptide comprises an amino sequence comprising at least one, two, or three but no more than four different amino acids, relative to the amino acid sequence of any one of SEQ ID NOs: 1725-3622.

In some embodiments, the peptide comprises an amino acid sequence comprising at least one, two, or three but no more than four modifications, e.g., substitutions (conservative substitutions), insertions, or deletions, relative to the amino acid sequence of any of SEQ ID NO: 3648-3659. In some embodiments, the peptide comprises an amino sequence comprising at least one, two, or three but no more than four different amino acids, relative to the amino acid sequence of any one of SEQ ID NOs: 3648-3659.

In some embodiments, the peptide comprises the amino acid sequence of PLNGAVHLY (SEQ ID NO: 3648); an amino acid sequence comprising at least one, two, or three but no more than four modifications, e.g., substitutions (conservative substitutions), insertions, or deletions, relative to the amino acid sequence of PLNGAVHLY (SEQ ID NO: 3648); or an amino acid sequence comprising at least one, two, or three but no more than four different amino acids relative to the amino acid sequence of PLNGAVHLY (SEQ ID NO: 3648), optionally wherein position 7 is H.

In some embodiments, the peptide comprises the amino acid sequence of RDSPKGW (SEQ ID NO: 3649), or an amino acid sequence comprising at least one, two, or three modifications but no more than four modifications, e.g., substitutions (conservative substitutions), insertions, or deletions, relative to the amino acid sequence of RDSPKGW (SEQ ID NO: 3649); or an amino acid sequence comprising at least one, two, or three but no more than four different amino acids relative to the amino acid sequence of RDSPKGW (SEQ ID NO: 3649).

In some embodiments, the peptide comprises the amino acid sequence of IVMNSLK (SEQ ID NO: 3651), or an amino acid sequence comprising at least one, two, or three modifications but no more than four modifications, e.g., substitutions (conservative substitutions), insertions, or deletions, relative to the amino acid sequence of IVMNSLK (SEQ ID NO: 3651); or an amino acid sequence comprising at least one, two, or three but no more than four different amino acids relative to the amino acid sequence of IVMNSLK (SEQ ID NO: 3651).

In some embodiments, the peptide comprises the amino acid sequence of YSTDVRM (SEQ ID NO: 3650), or an amino acid sequence comprising at least one, two, or three modifications but no more than four modifications, e.g., substitutions (conservative substitutions), insertions, or deletions, relative to the amino acid sequence of YSTDVRM (SEQ ID NO: 3650); or an amino acid sequence comprising at least one, two, or three but no more than four different amino acids relative to the amino acid sequence of YSTDVRM (SEQ ID NO: 3650).

In some embodiments, the peptide comprises the amino acid sequence of RESPRGL (SEQ ID NO: 3652), or a sequence comprising at least one, two, or three modifications but no more than four modifications, e.g., substitutions (conservative substitutions), insertions, or deletions, relative to the amino acid sequence of RESPRGL (SEQ ID NO: 3652); or an amino acid sequence comprising at least one, two, or three but no more than four different amino acids relative to the amino acid sequence of RESPRGL (SEQ ID NO: 3652).

In some embodiments, the peptide comprises the amino acid sequence of any of SEQ ID NOs: 139-1138. In some embodiments, the peptide comprises the amino acid sequence of any of SEQ ID NOs: 139-476. In some embodiments, the peptide comprises the amino acid sequence of any one of the amino acid sequences provided in Table 1B. In some embodiments, the peptide comprises the amino acid sequence of any of SEQ ID NOs: 1139-1172.

In some embodiments, the peptide comprises the amino acid sequence of any of SEQ ID NO: 1725-3622. In some embodiments, the peptide comprises the amino acid sequence of any of SEQ ID NO: 3648-3659.

In some embodiments, the peptide may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the sequences shown in Table 1A, Table 1B, Table 2, Table 7, Table 10, Table 11, or Table 20.

In some embodiments, the peptide comprises the amino acid sequence of SEQ ID NO: 3648. In some embodiments, the peptide comprises the amino acid sequence of SEQ ID NO: 3649. In some embodiments, the peptide comprises the amino acid sequence of SEQ ID NO: 3650. In some embodiments, the peptide comprises the amino acid sequence of SEQ ID NO: 3651. In some embodiments, the peptide comprises the amino acid sequence of SEQ ID NO: 3652. In some embodiments, the peptide comprises the amino acid sequence of SEQ ID NO: 3653. In some embodiments, the peptide comprises the amino acid sequence of SEQ ID NO: 3654. In some embodiments, the peptide comprises the amino acid sequence of SEQ ID NO: 3655. In some embodiments, the peptide comprises the amino acid sequence of SEQ ID NO: 3656. In some embodiments, the peptide comprises the amino acid sequence of SEQ ID NO: 3657. In some embodiments, the peptide comprises the amino acid sequence of SEQ ID NO: 3658. In some embodiments, the peptide comprises the amino acid sequence of SEQ ID NO: 3659.

In some embodiments, the peptide may comprise SEQ ID NO: 1725. In some embodiments, the peptide may comprise SEQ ID NO: 1726. In some embodiments, the peptide may comprise SEQ ID NO: 1729. In some embodiments, the peptide may comprise SEQ ID NO: 1760. In some embodiments, the peptide may comprise SEQ ID NO: 1769. In some embodiments, the peptide may comprise SEQ ID NO: 3622. In some embodiments, the peptide may comprise SEQ ID NO: 1798. In some embodiments, the peptide may comprise SEQ ID NO: 1785. In some embodiments, the peptide may comprise SEQ ID NO: 1767. In some embodiments, the peptide may comprise SEQ ID NO: 1734. In some embodiments, the peptide may comprise SEQ ID NO: 1737. In some embodiments, the peptide may comprise SEQ ID NO: 1819.

In some embodiments, the peptide comprises an amino acid sequence encoded by a nucleotide sequence described herein, e.g., a nucleotide sequence of Table 2. In some embodiments, the peptide comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the peptide comprises an amino acid sequence encoded by a nucleotide sequence comprising at least one, two, three, four, five, six, or seven, but no more than ten different nucleotides relative to the nucleotide sequence of any of SEQ ID NOs: 3660-3671. In some embodiments, the peptide comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto.

In some embodiments, the peptide comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of SEQ ID NO: 3660. In some embodiments, the peptide comprises an amino acid sequence encoded by a nucleotide sequence comprising at least one, two, three, four, five, six, or seven, but no more than ten different nucleotides relative to the nucleotide sequence of SEQ ID NO: 3660. In some embodiments, the peptide comprises an amino acid sequence encoded by the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto.

In some embodiments, the peptide comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of SEQ ID NO: 3663. In some embodiments, the peptide comprises an amino acid sequence encoded by a nucleotide sequence comprising at least one, two, three, four, five, six, or seven, but no more than ten different nucleotides relative to the nucleotide sequence of SEQ ID NO: 3663. In some embodiments, the peptide comprises an amino acid sequence encoded by the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding a peptide described herein e.g., peptide 1-12, comprises a nucleotide sequence described herein, e.g., as described in Table 2. In some embodiments, the nucleic acid sequence encoding a peptide described herein comprises the nucleotide sequence of any of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding a peptide described herein comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the nucleotide sequence encoding the peptide comprises a nucleotide sequence comprising at least one, two, three, four, five, six, or seven, but no more than ten different nucleotides relative to the nucleotide sequence of any of SEQ ID NOs: 3660-367. In some embodiments, the nucleotide sequence encoding a peptide described herein is isolated, e.g., recombinant.

In some embodiments the nucleotide sequence encoding a peptide described herein comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of SEQ ID NO: 3660. In some embodiments, the nucleotide sequence encoding the peptide comprises a nucleotide sequence comprising at least one, two, three, four, five, six, or seven, but no more than ten different nucleotides relative to the nucleotide sequence of SEQ ID NO: 3660. In some embodiments the nucleic acid sequence encoding a peptide described herein comprises a nucleotide sequence comprising the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto.

In some embodiments, the nucleic acid encoding a peptide described herein comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of SEQ ID NO: 3663. In some embodiments, the nucleotide sequence encoding the peptide comprises a nucleotide sequence comprising at least one, two, three, four, five, six, or seven, but no more than ten different nucleotides relative to the nucleotide sequence of SEQ ID NO: 3663. In some embodiments the nucleic acid encoding a peptide described herein comprises a nucleotide sequence comprising the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto.

In some embodiments, a peptide described herein is fused or coupled, e.g., conjugated, to an active agent. In some embodiments, the active agent is a therapeutic agent. In some embodiments, the agent is a therapeutic agent. In some embodiments, the active agent comprises a therapeutic protein, an antibody molecule, an enzyme, one or more components of a genome editing system, an Fc polypeptide fused or coupled (e.g., covalently or non covalently) to a therapeutic agent, and/or an RNAi agent (e.g., a dsRNA, antisense oligonucleotide (ASO), siRNA, shRNA, pre-miRNA, pri-miRNA, miRNA, stRNA, lncRNA, piRNA, or snoRNA). In some embodiments, the therapeutic agent is an antibody. In some embodiments, the peptide is fused or coupled, e.g., conjugated (e.g., directly or indirectly) to the Fc region of the antibody, e.g., at the C-terminus of the Fc region or the N-terminus of the Fc region. In some embodiments, the therapeutic agent is an RNAi agent. In some embodiments, the RNAi agent is a siRNA or an ASO. In some embodiments, the ASO or siRNA comprises at least one (e.g., one or more or all) modified nucleotides. In some embodiments, the peptide is fused or coupled, e.g., conjugated (e.g., directly or indirectly via a linker), to at least one strand of the RNAi agent. In some embodiments, the peptide is conjugated, e.g., directly or indirectly via a linker, to the C-terminus of at least one strand of the RNAi agent. In some embodiments, the peptide is conjugated, e.g., directly or indirectly via a linker, to an internal nucleotide of at least one strand of the RNAi agent. In some embodiments, the at least one strand is the sense strand. In some embodiments, the therapeutic agent modulates, e.g., inhibits, decreases, or increases, expression of, a CNS related gene, mRNA, and/or protein.

In some embodiments, the active agent is a diagnostic agent. In some embodiments, the diagnostic agent is or comprises an imaging agent (e.g., a protein or small molecule compound coupled to a detectable moiety). In some embodiments, the imaging agent comprises a PET or MRI ligand, or an antibody molecule coupled to a detectable moiety. In some embodiments, the detectable moiety is or comprises a radiolabel, a fluorophore, a chromophore, or an affinity tag. In some embodiments, the radiolabel is or comprises tc99m, iodine-123, a spin label, iodine-131, indium-111, fluorine-19, carbon-13, nitrogen-15, oxygen-17, gadolinium, manganese, or iron. In some embodiments, the active agent is a small molecule. In some embodiments, the active agent is a ribonucleic acid complex (e.g., a Cas9/gRNA complex), a plasmid, a closed-end DNA, a circ-RNA, or an mRNA.

In some embodiments, at least 1-5, e.g., at least 1, 2, 3, 4, or 5, peptides are fused or coupled, e.g., conjugated, to an active agent, e.g., a therapeutic agent or a diagnostic agent. In some embodiments, the at least 1-5, e.g., at least 1, 2, 3, 4, or 5, peptides comprise the same amino acid sequence. In some embodiments, the at least 1-5, e.g., at least 1, 2, 3, 4, or 5, peptides comprise different amino acid sequences. In some embodiments, the at least 1-5, e.g., at least 1, 2, 3, 4, or 5, peptides are present in tandem (e.g., connected directly or indirectly via a linker) or in a multimeric configuration. In some embodiments, the peptide comprises an amino acid sequence of at least 3, 4, 5, 6, 7, 8, 9, 10, 11, 12, 15, 20, 25, 30, or 35 amino acids in length.

In some embodiments, the peptide covalently linked, e.g., directly or indirectly via a linker, to the active agent. In some embodiments, the peptide is conjugated to the active agent via a linker. In some embodiments, the linker is a cleavable linker or a non-cleavable linker. In some embodiments, the cleavable linker is a pH sensitive linker or an enzyme sensitive linker. In some embodiments, the pH sensitive linker comprises a hydrazine/hydrazone linker or a disulfide linker. In some embodiments, the enzyme sensitive linker comprises a peptide based linker, e.g., a peptide linker sensitive to a protease (e.g., a lysosomal protease); or a beta-glucuronide linker. In some embodiments, the non-cleavable linker is a linker comprising a thioether group or a maleimidocaproyl group. In some embodiments, the peptide and the active agent are fused or coupled post-translationally, e.g., using click chemistry. In some embodiments, the peptide and the active agent are fused or couple via chemically induced dimerization. In some embodiments, the peptide is present N-terminal relative to the active agent. In some embodiments, the peptide is present C-terminal relative to the active agent.

In some embodiments, the peptide is present or coupled to a carrier. In some embodiments, the carrier comprises an exosome, a microvesicle, or a lipid nanoparticle (LNP). In some embodiments, the carrier comprises a therapeutic agent (e.g., an RNAi agent (e.g., an dsRNA, a siRNA, a shRNA, a pre-miRNA, a pri-miRNA, a miRNA, a stRNA, a lncRNA, a piRNA, an antisense oligonucleotide agent (ASO), or a snoRNA), an mRNA, a ribonucleoprotein complex (e.g., a Cas9/gRNA complex), or a circRNA). In some embodiments, the peptide is present on the surface of the carrier. In some embodiments, at least 10%, 20%, 30%, 40%, 50%, 60%, 70%, or 80% of the surface of the carrier comprises at least 1-5, e.g., at least 1, 2, 3, 4, or 5 peptides described herein.

The present disclosure also provides a nucleic acid or polynucleotide encoding any of the peptides described herein, and AAV capsid polypeptides, e.g., AAV capsid variants, AAV particles, vectors, cells, and formulations, e.g., pharmaceutical formulations, comprising the same.

### AAV Capsid Polypeptide, e.g., AAV Capsid Variant

In some embodiments, an AAV particle described herein comprises an AAV capsid polypeptide, e.g., an AAV capsid polypeptide, e.g., an AAV capsid variant. In some embodiments, the AAV capsid variant comprises a peptide, sequence as described in Table 1A, 1B, 2, 7, 10, 11, or 20.

In some embodiments, the AAV capsid polypeptide, e.g., the AAV capsid variant, comprises an amino acid sequence comprising the following formula [N1]-[N2], wherein [N1] comprises X1, X2, X3, X4, and X5, and [N2] comprises the amino acid sequence of VHLY (SEQ ID NO: 4680), VHIY (SEQ ID NO: 4681), VHVY (SEQ ID NO: 4682), or VHHY (SEQ ID NO: 4683). In some embodiments, position X1 of [N1] is: P, Q, A, H, K, L, R, S, or T. In some embodiments, position X2 of [N1] is: L, I, V, H, or R. In some embodiments, position X3 of [N1] is: N, D, I, K, or Y. In some embodiments, position X4 of [N1] is: G, A, C, R, or S. In some embodiments, position X5 of [N1] is: A, S, T, G, C, D, N, Q, V, or Y. In some embodiments [N1] comprises AL, PI, PL, QL, SL, TL, LN, LD, IN, NG, DG, DS, GA, SA, SS, GG, GN, GS, or GT. In some embodiments, [N1] comprises ALD, ALN, PIN, PLD, PLN, QLN, SLD, SLN, TLN, LNG, LDG, ING, LDS, NGA, DGA, DSA, DSS, NGG, NGN, NGS, NGT. In some embodiments, [N1] is or comprises SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGA (SEQ ID NO: 3679), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722). In some embodiments, [N1] is or comprises ALDGA (SEQ ID NO: 4698), ALNGA (SEQ ID NO: 4686), PINGA (SEQ ID NO: 4697), PLDGA (SEQ ID NO: 4691), PLDSA (SEQ ID NO: 4701), PLDSS (SEQ ID NO: 4705), PLNGA (SEQ ID NO: 3679), PLNGG (SEQ ID NO: 4689), PLNGN (SEQ ID NO: 4693), PLNGS (SEQ ID NO: 4687), PLNGT (SEQ ID NO: 4690), QLNGA (SEQ ID NO: 4685), SLDGA (SEQ ID NO: 4694), SLNGA (SEQ ID NO: 4684), or TLNGA (SEQ ID NO: 4708). In some embodiments, wherein [N1]-[N2] comprises LDGAVHLY (SEQ ID NO: 4768), LNGAVHLY (SEQ ID NO: 4769), INGAVHLY (SEQ ID NO: 4770), LDSAVHLY (SEQ ID NO: 4771), LDSSVHLY (SEQ ID NO: 4772), LNGGVHLY (SEQ ID NO: 4773), LNGNVHLY (SEQ ID NO: 4774), LNGSVHLY (SEQ ID NO: 4775), LNGTVHLY (SEQ ID NO: 4776), LNGAVHIY (SEQ ID NO: 4777), LDGAVHVY (SEQ ID NO: 4778), LNGAVHHY (SEQ ID NO: 4779). In some embodiments, [N1]-[N2] is or comprises ALDGAVHLY (SEQ ID NO: 4780), ALNGAVHLY (SEQ ID NO: 4781), PINGAVHLY (SEQ ID NO: 4782), PLDGAVHLY (SEQ ID NO: 4783), PLDSAVHLY (SEQ ID NO: 4784), PLDSSVHLY (SEQ ID NO: 4785), PLNGAVHLY (SEQ ID NO: 3648), PLNGGVHLY (SEQ ID NO: 4786), PLNGNVHLY (SEQ ID NO: 4787), PLNGSVHLY (SEQ ID NO: 4788), PLNGTVHLY (SEQ ID NO: 4789), QLNGAVHLY (SEQ ID NO: 4790), SLDGAVHLY (SEQ ID NO: 4791), SLNGAVHLY (SEQ ID NO: 4792), TLNGAVHLY (SEQ ID NO: 4793), PLNGAVHIY (SEQ ID NO: 4794), PLDGAVHVY (SEQ ID NO: 4795), PLNGAVHHY (SEQ ID NO: 4796); an amino acid sequence comprising any portion of any of the aforesaid amino acid sequences (e.g., any 2, 3, 4, 5, 6, 7, or 8 amino acids, e.g., consecutive amino acids) thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, the AAV capsid polypeptide, e.g., the AAV capsid variant, comprising the amino acid sequence comprising the formula of [N1]-[N2], further comprises [N3], which comprises X6, X7, X8, and X9. In some embodiments, position X6 of [N3] is: A, D, S, or T. In some embodiments, position X7 of [N3] is: Q, K, H, L, P, or R. In some embodiments, position X8 of [N3] is: A, P, E, or R. In some embodiments, position X9 of [N3] is Q, H, K, or P. In some embodiments, [N3] comprises AQ, SQ, AK, DQ, PQ, QA, QP, or KA. In some embodiments, [N3] comprises AQA, AQP, SQA, AKA, DQA, QAQ, QPQ, or KAQ. In some embodiments, [N3] is or comprises AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751). In some embodiments, [N3] is or comprises AQAQ (SEQ ID NO: 4737), AQPQ (SEQ ID NO: 4739), SQAQ (SEQ ID NO: 4738), AKAQ (SEQ ID NO: 4741), or DQAQ (SEQ ID NO: 4744). In some embodiments, is or comprises ALDGAVHLYAQAQ (SEQ ID NO: 4827), ALNGAVHLYAQAQ (SEQ ID NO: 4828), PINGAVHLYAQAQ (SEQ ID NO: 4829), PLDGAVHLYAQAQ (SEQ ID NO: 4830), PLDGAVHLYAQPQ (SEQ ID NO: 4831), PLDGAVHLYSQAQ (SEQ ID NO: 4832), PLDSAVHLYAQAQ (SEQ ID NO: 4833), PLDSSVHLYAQAQ (SEQ ID NO: 4834), PLNGAVHLYAKAQ (SEQ ID NO: 4835), PLNGAVHLYAQAQ (SEQ ID NO: 4836), PLNGAVHLYAQPQ (SEQ ID NO: 4837), PLNGAVHLYDQAQ (SEQ ID NO: 4838), PLNGAVHLYSQAQ (SEQ ID NO: 4839), PLNGGVHLYAQAQ (SEQ ID NO: 4840), PLNGNVHLYAQAQ (SEQ ID NO: 4841), PLNGSVHLYAQAQ (SEQ ID NO: 4842), PLNGTVHLYAQAQ (SEQ ID NO: 4843), QLNGAVHLYAQAQ (SEQ ID NO: 4844), SLDGAVHLYAQAQ (SEQ ID NO: 4845), SLNGAVHLYAQAQ (SEQ ID NO: 4846), TLNGAVHLYAQAQ (SEQ ID NO: 4847), PLNGAVHIYAQAQ (SEQ ID NO: 4848), PLDGAVHVYAQAQ (SEQ ID NO: 4849), PLNGAVHHYAQAQ (SEQ ID NO: 4850); an amino acid sequence comprising any portion of any of the aforesaid amino acid sequences (e.g., any 2, 3, 4, 5, 6, 7, 8, 9, 10, 11, or 12 amino acids, e.g., consecutive amino acids) thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, the AAV capsid polypeptide, e.g., the AAV capsid variant, comprising the amino acid sequence comprising the formula [N1]-[N2], further comprises [N4], which comprises X10, X11, and X12. In some embodiments, position X10 of [N4] is: T, V, L, R, S, A, C, I, K, M, N, P, or Q. In some embodiments, position X1I of [N4] is: G, S, A, T, M, V, Q, L, H, I, K, N, P, R, or Y. In some embodiments, position X12 of [N4] is: W, S, P, G, A, Q, L, M, K, C, E, F, H, R, T, V, or Y. In some embodiments, [N4] comprises LS, TG, LA, LT, SA, SS, TL, TT, TS, TA, TV, VS, AA, AG, AS, AT, CS, CT, IA, IG, IL, IQ, IS, IT, LG, LH, LK, LM, LN, LQ, MA, NA, NM, NS, NT, NV, QA, RA, RG, RI, RL, RM, RN, RQ, RS, RT, RV, SG, SM, ST, SV, TK, TM, TN, TP, TQ, TR, VA, VG, VH, VK, VL, VM, VN, VQ, VR, VT, PG, LV, SP, GW, AP, GR, AL, AW, GG, GS, GP, QP, QS, AH, AN, AQ, AR, GQ, HP, KS, MG, MP, MQ, MS, NP, QQ, QR, SH, SK, SQ, SR, IP, VE, AK, AM, AV, GA, GC, GT, KA, KP, KQ, LP, MK, MN, MT, NQ, PP, QH, QK, QM, QN, QT, RW, SL, VW, GK, GN, NG, RP, SN, GL, or VP. In some embodiments, [N4] is or comprises TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW.

In some embodiments, the AAV capsid polypeptide, e.g., the AAV capsid variant, comprising the amino acid sequence comprising the formula [N1]-[N2], further comprises [N5] which comprises X13, X14, and X15. In some embodiments, position X13 of [N5] is: V, D, F, G, L, A, E, or I. In some embodiments, position X14 of [N5] is: Q, K, R, H, E, L, or P. In some embodiments, position X15 of [N5] is: N, T, K, H, D, Y, S, I, or P. In some embodiments, [N5] comprises VQ, AQ, DQ, FQ, VL, LQ, EQ, GQ, VP, VR, VK, QN, QS, QT, QK, QH, LN, QI, PN, QD, QP, RN, or KN. In some embodiments, [N5] is or comprises VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH. In some embodiments [N5] is or comprises VQN, AQN, VQS, DQN, VQT, VQK, VQH, FQN, VLN, LQN, VQI, EQN, GQT, VPN, VQD, VQP, VRN, or VKN. In some embodiments [N4]-[N5] is or comprises TGWVQN (SEQ ID NO: 4851), LAAVQN (SEQ ID NO: 4852), LTPVQN (SEQ ID NO: 4853), SAPVQN (SEQ ID NO: 4854), SSPVQN (SEQ ID NO: 4855), TGRVQN (SEQ ID NO: 4856), TGWAQN (SEQ ID NO: 4857), TGWVQS (SEQ ID NO: 4858), TLAVQN (SEQ ID NO: 4859), TTSVQN (SEQ ID NO: 4860), TSPVQN (SEQ ID NO: 4861), TALVQN (SEQ ID NO: 4862), TAWVQN (SEQ ID NO: 4863), TGGVQN (SEQ ID NO: 4864), TGSVQN (SEQ ID NO: 4865), TGWDQN (SEQ ID NO: 4866), TVSVQN (SEQ ID NO: 4867), VSPVQN (SEQ ID NO: 4868), VSSVQN (SEQ ID NO: 4869), AAPVQN (SEQ ID NO: 4870), AGPVQN (SEQ ID NO: 4871), ASPVQN (SEQ ID NO: 4872), ATPVQN (SEQ ID NO: 4873), CSPVQN (SEQ ID NO: 4874), CTPVQN (SEQ ID NO: 4875), IAAVQN (SEQ ID NO: 4876), IAGVQN (SEQ ID NO: 4877), IASVQN (SEQ ID NO: 4878), IGGVQN (SEQ ID NO: 4879), IGSVQN (SEQ ID NO: 4880), ILGVQN (SEQ ID NO: 4881), IQPVQN (SEQ ID NO: 4882), IQSVQN (SEQ ID NO: 4883), ISGVQN (SEQ ID NO: 4884), ISPVQN (SEQ ID NO: 4885), ISSVQN (SEQ ID NO: 4886), ITPVQN (SEQ ID NO: 4887), LAGVQN (SEQ ID NO: 4888), LAHVQN (SEQ ID NO: 4889), LANVQN (SEQ ID NO: 4890), LAPVQN (SEQ ID NO: 4891), LAPVQT (SEQ ID NO: 4892), LAQVQN (SEQ ID NO: 4893), LARVQN (SEQ ID NO: 4894), LASVQN (SEQ ID NO: 4895), LATVQN (SEQ ID NO: 4896), LGPVQN (SEQ ID NO: 4897), LGQVQN (SEQ ID NO: 4898), LGSVQN (SEQ ID NO: 4899), LHPVQN (SEQ ID NO: 4900), LKSVQN (SEQ ID NO: 4901), LMAVQN (SEQ ID NO: 4902), LMGVQN (SEQ ID NO: 4903), LMPVQN (SEQ ID NO: 4904), LMQVQN (SEQ ID NO: 4905), LMSVQN (SEQ ID NO: 4906), LNPVQN (SEQ ID NO: 4907), LQPVQN (SEQ ID NO: 4908), LQQVQN (SEQ ID NO: 4909), LQRVQN (SEQ ID NO: 4910), LSHVQN (SEQ ID NO: 4911), LSKVQN (SEQ ID NO: 4912), LSPVQK (SEQ ID NO: 4913), LSPVQN (SEQ ID NO: 4914), LSQVQN (SEQ ID NO: 4915), LSRVQN (SEQ ID NO: 4916), LSTVQN (SEQ ID NO: 4917), LTAVQN (SEQ ID NO: 4918), LTNVQN (SEQ ID NO: 4919), LTSVQN (SEQ ID NO: 4920), MAPVQN (SEQ ID NO: 4921), NAQVQN (SEQ ID NO: 4922), NASVQN (SEQ ID NO: 4923), NMQVQN (SEQ ID NO: 4924), NSPVQN (SEQ ID NO: 4925), NTPVQN (SEQ ID NO: 4926), NVQVQN (SEQ ID NO: 4927), QAPVQN (SEQ ID NO: 4928), RAAVQN (SEQ ID NO: 4929), RAQVQN (SEQ ID NO: 4930), RASVQN (SEQ ID NO: 4931), RGGVQN (SEQ ID NO: 4932), RGSVQN (SEQ ID NO: 4933), RIAVQN (SEQ ID NO: 4934), RIGVQN (SEQ ID NO: 4935), RIPVQN (SEQ ID NO: 4936), RLGVQN (SEQ ID NO: 4937), RLSVQN (SEQ ID NO: 4938), RMSVQN (SEQ ID NO: 4939), RNSVQN (SEQ ID NO: 4940), RQPVQN (SEQ ID NO: 4941), RSAVQN (SEQ ID NO: 4942), RSGVQN (SEQ ID NO: 4943), RSPVQN (SEQ ID NO: 4944), RSQVQN (SEQ ID NO: 4945), RSSVQN (SEQ ID NO: 4946), RSTVQN (SEQ ID NO: 4947), RTAVQN (SEQ ID NO: 4948), RTGVQN (SEQ ID NO: 4949), RTLVQN (SEQ ID NO: 4950), RTSVQN (SEQ ID NO: 4951), RTTVQN (SEQ ID NO: 4952), RVEVQN (SEQ ID NO: 4953), SAAVQN (SEQ ID NO: 4954), SAKVQN (SEQ ID NO: 4955), SAMVQN (SEQ ID NO: 4956), SAQVQN (SEQ ID NO: 4957), SGPVQN (SEQ ID NO: 4958), SMAVQN (SEQ ID NO: 4959), SMGVQN (SEQ ID NO: 4960), SMQVQN (SEQ ID NO: 4961), SMSVQN (SEQ ID NO: 4962), STPVQN (SEQ ID NO: 4963), SVAVQN (SEQ ID NO: 4964), SVGVQN (SEQ ID NO: 4965), TAAVQN (SEQ ID NO: 4966), TAGVQN (SEQ ID NO: 4967), TAKVQN (SEQ ID NO: 4968), TAMVQN (SEQ ID NO: 4969), TANVQN (SEQ ID NO: 4970), TAPVQN (SEQ ID NO: 4971), TAPVQT (SEQ ID NO: 4972), TAQVQN (SEQ ID NO: 4973), TASVQN (SEQ ID NO: 4974), TASVQT (SEQ ID NO: 4975), TATVQN (SEQ ID NO: 4976), TAVVQN (SEQ ID NO: 4977), TAWDQN (SEQ ID NO: 4978), TAWVQH (SEQ ID NO: 4979), TAWVQT (SEQ ID NO: 4980), TGAVQN (SEQ ID NO: 4981), TGCFQN (SEQ ID NO: 4982), TGGAQN (SEQ ID NO: 4983), TGGFQN (SEQ ID NO: 4984), TGGVLN (SEQ ID NO: 4985), TGGVQH (SEQ ID NO: 4986), TGGVQK (SEQ ID NO: 4987), TGGVQT (SEQ ID NO: 4988), TGPVQN (SEQ ID NO: 4989), TGSAQN (SEQ ID NO: 4990), TGSLQN (SEQ ID NO: 4991), TGSVQH (SEQ ID NO: 4992), TGSVQI (SEQ ID NO: 4993), TGSVQS (SEQ ID NO: 4994), TGSVQT (SEQ ID NO: 4995), TGTVQN (SEQ ID NO: 4996), TGWEQN (SEQ ID NO: 4997), TGWFQN (SEQ ID NO: 4998), TGWGQT (SEQ ID NO: 4999), TGWVPN (SEQ ID NO: 5000), TGWVQD (SEQ ID NO: 5001), TGWVQP (SEQ ID NO: 5002), TGWVQT (SEQ ID NO: 5003), TGWVRN (SEQ ID NO: 5004), TKAVQN (SEQ ID NO: 5005), TKPVQN (SEQ ID NO: 5006), TKQVQN (SEQ ID NO: 5007), TKSVQN (SEQ ID NO: 5008), TLPVQN (SEQ ID NO: 5009), TLQVQN (SEQ ID NO: 5010), TMAVQN (SEQ ID NO: 5011), TMGVQN (SEQ ID NO: 5012), TMKVQN (SEQ ID NO: 5013), TMNVQN (SEQ ID NO: 5014), TMPVQN (SEQ ID NO: 5015), TMQVQN (SEQ ID NO: 5016), TMSVKN (SEQ ID NO: 5017), TMSVQN (SEQ ID NO: 5018), TMSVQT (SEQ ID NO: 5019), TMTVQN (SEQ ID NO: 5020), TNAVQN (SEQ ID NO: 5021), TNQVQN (SEQ ID NO: 5022), TNSVQN (SEQ ID NO: 5023), TPPVQN (SEQ ID NO: 5024), TQHVQN (SEQ ID NO: 5025), TQKVQN (SEQ ID NO: 5026), TQMVQN (SEQ ID NO: 5027), TQNVQN (SEQ ID NO: 5028), TQPVQN (SEQ ID NO: 5029), TQQVQN (SEQ ID NO: 5030), TQTVQN (SEQ ID NO: 5031), TRWDQN (SEQ ID NO: 5032), TSAVQN (SEQ ID NO: 5033), TSGVQN (SEQ ID NO: 5034), TSHVQN (SEQ ID NO: 5035), TSKVQN (SEQ ID NO: 5036), TSLVQN (SEQ ID NO: 5037), TSMVQN (SEQ ID NO: 5038), TSPDQN (SEQ ID NO: 5039), TSQVQN (SEQ ID NO: 5040), TSSVQN (SEQ ID NO: 5041), TSSVQT (SEQ ID NO: 5042), TSTVQN (SEQ ID NO: 5043), TSVVQN (SEQ ID NO: 5044), TTAVQN (SEQ ID NO: 5045), TTGVQN (SEQ ID NO: 5046), TTKVQN (SEQ ID NO: 5047), TTPVQN (SEQ ID NO: 5048), TTPVQT (SEQ ID NO: 5049), TTQVQN (SEQ ID NO: 5050), TTTVQN (SEQ ID NO: 5051), TVAVQN (SEQ ID NO: 5052), TVAVQT (SEQ ID NO: 5053), TVGVQN (SEQ ID NO: 5054), TVQVQN (SEQ ID NO: 5055), TVSVKN (SEQ ID NO: 5056), TVWVQK (SEQ ID NO: 5057), VAAVQN (SEQ ID NO: 5058), VAGVQN (SEQ ID NO: 5059), VAKVQN (SEQ ID NO: 5060), VANVQN (SEQ ID NO: 5061), VAQVQN (SEQ ID NO: 5062), VASVQN (SEQ ID NO: 5063), VATVQN (SEQ ID NO: 5064), VGGVQN (SEQ ID NO: 5065), VGKVQN (SEQ ID NO: 5066), VGNVQN (SEQ ID NO: 5067), VGSVQN (SEQ ID NO: 5068), VHPVQN (SEQ ID NO: 5069), VKAVQN (SEQ ID NO: 5070), VKPVQN (SEQ ID NO: 5071), VKQVQN (SEQ ID NO: 5072), VLPVQN (SEQ ID NO: 5073), VLSVQN (SEQ ID NO: 5074), VMAVQN (SEQ ID NO: 5075), VMQVQN (SEQ ID NO: 5076), VMSVQN (SEQ ID NO: 5077), VNAVQN (SEQ ID NO: 5078), VNGVQN (SEQ ID NO: 5079), VNSVQN (SEQ ID NO: 5080), VQAVQN (SEQ ID NO: 5081), VQNVQN (SEQ ID NO: 5082), VQPVQN (SEQ ID NO: 5083), VQQVQN (SEQ ID NO: 5084), VQSVQN (SEQ ID NO: 5085), VQTVQN (SEQ ID NO: 5086), VRPVQN (SEQ ID NO: 5087), VSAVQN (SEQ ID NO: 5088), VSGVQN (SEQ ID NO: 5089), VSNVQN (SEQ ID NO: 5090), VSPVQT (SEQ ID NO: 5091), VSQVQN (SEQ ID NO: 5092), VSRVQN (SEQ ID NO: 5093), VSSVQK (SEQ ID NO: 5094), VSSVQT (SEQ ID NO: 5095), VSTVQN (SEQ ID NO: 5096), VTAVQN (SEQ ID NO: 5097), VTGVQN (SEQ ID NO: 5098), VTKVQN (SEQ ID NO: 5099), VTPVQN (SEQ ID NO: 5100), VTSVQN (SEQ ID NO: 5101), TGLVQN (SEQ ID NO: 5102), TGWVKN (SEQ ID NO: 5103), PGWVQN (SEQ ID NO: 5104), TGWVQH (SEQ ID NO: 5105), LSGVQN (SEQ ID NO: 5106), LSSVQN (SEQ ID NO: 5107), LVPVQN (SEQ ID NO: 5108); an amino acid sequence comprising any portion of any of the aforesaid amino acid sequences (e.g., any 2, 3, 4, 5, or 6 amino acids, e.g., consecutive amino acids) thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, [N1]-[N2]-[N3]-[N4]-[N5] of the AAV capsid variant comprises the amino acid sequence of any of SEQ ID NOs: 139-1138; an amino acid sequence comprising at least one, two, or three but no more than four modifications, e.g., substitutions (e.g., conservative substitutions), insertions, or deletions, relative to any of SEQ ID NOs: 139-1138; or an amino acid sequence comprising at least one, two, or three but no more than four different amino acids, relative to any one of SEQ ID NOs: 139-1138. In some embodiments, [N1]-[N2]-[N3]-[N4]-[N5] comprises the amino acid sequence of any of SEQ ID NOs: 139-476; an amino acid sequence comprising at least one, two, or three but no more than four modifications, e.g., substitutions (e.g., conservative substitutions), insertions, or deletions, relative to any of SEQ ID NOs: 139-476; or an amino acid sequence comprising at least one, two, or three but no more than four different amino acids, relative to any one of SEQ ID NOs: 139-476.

In some embodiments, [N1]-[N2] is present in loop VIII of the AAV capsid variant. In some embodiments, [N3], [N4], and/or [N5] are present in loop VIII of the AAV capsid variant. In some embodiments, [N] is present immediately subsequent to position 586, relative to a reference sequence numbered according to the amino acid sequence of SEQ ID NO: 138. In some embodiments, [N2] is present immediately subsequent to [N1]. In some embodiments, [N3] is present immediately subsequent to position 588, relative to a reference sequence numbered according to the amino acid sequence of SEQ ID NO: 138. In some embodiments, [N4] is present immediately subsequent to position 592, relative to a reference sequence numbered according to the amino acid sequence of SEQ ID NO: 138. In some embodiments, [N5] is present immediately subsequent to position 595, relative to a reference sequence numbered according to the amino acid sequence of SEQ ID NO: 138. in some embodiments, [N1]-[N2]-[N3]-[N4]-[N5] is present immediately subsequent to position 586, relative to a reference sequence numbered according to the amino acid sequence of SEQ ID NO: 138.

In some embodiments, the AAV capsid polypeptide, e.g., the AAV capsid variant, comprises from N to C-terminus, [N1]-[N2]. In some embodiments, the AAV capsid polypeptide, e.g., the AAV capsid variant, comprises from N to C-terminus, [N1]-[N2]-[N3]. In some embodiments, the AAV capsid polypeptide, e.g., the AAV capsid variant, comprises from N to C-terminus, [N1]-[N2]-[N3]-[N4]. In some embodiments, the AAV capsid polypeptide, e.g., the AAV capsid variant, comprises from N to C-terminus, [N1]-[N2]-[N3]-[N4]-[N5].

In some embodiments, the AAV capsid polypeptide, e.g., the AAV capsid variant, comprises an amino acid sequence having the formula [A][B], wherein [A] comprises the amino acid sequence of PLNGA (SEQ ID NO: 3679), and [B] comprises X1, X2, X3, and X4. In some embodiments, position X1 of [B] is: V, I, L, A, F, D, or G. In some embodiments, position X2 of [B] is H, N, Q, P, D, L, R, or Y. In some embodiments, position X3 of [B] is L, H, I, R, or V. In some embodiments, position X4 of [B] is Y. In some embodiments, [B] comprises VH, VN, VQ, IH, LH, VP, VD, AH, FH, DH, VL, GH, VR, VY, LY, HY, IY, RY, HL, HH, HI, NL, QL, PL, DL, HR, LL, RL, HV, or YL. In some embodiments, B comprises VHL, VHH, VHI, VNL, VQL, IHL, LHL, VPL, VDL, AHL, VHR, FHL, DHL, VLL, GHL, VRL, VHV, VYL, HLY, HHY, HIY, NLY, QLY, PLY, DLY, HRY, LLY, RLY, HVY, YLY. In some embodiments, [B] is or comprises VHLY (SEQ ID NO: 4680), VHHY (SEQ ID NO: 4683), VHIY (SEQ ID NO: 4681), VNLY (SEQ ID NO: 4724), VQLY (SEQ ID NO: 4729), IHLY (SEQ ID NO: 4730), LHLY (SEQ ID NO: 4727), VPLY (SEQ ID NO: 4723), VDLY (SEQ ID NO: 4731), AHLY (SEQ ID NO: 4732), VHRY (SEQ ID NO: 4725), FHLY (SEQ ID NO: 4726), DHLY (SEQ ID NO: 4728), VLLY (SEQ ID NO: 4733), GHLY (SEQ ID NO: 4734), VRLY (SEQ ID NO: 4735), VHVY (SEQ ID NO: 4682), or VYLY (SEQ ID NO: 4736). In some embodiments, [B] is or comprises VHLY (SEQ ID NO: 4680), VHHY (SEQ ID NO: 4683), or VHIY (SEQ ID NO: 4681). In some embodiments, [A][B] is or comprises PLNGAVHLY (SEQ ID NO: 3648), PLNGAVHHY (SEQ ID NO: 4796), PLNGAVHIY (SEQ ID NO: 4794), PLNGAVNLY (SEQ ID NO: 5123), PLNGAVQLY (SEQ ID NO: 5124), PLNGAIHLY (SEQ ID NO: 5125), PLNGALHLY (SEQ ID NO: 5126), PLNGAVPLY (SEQ ID NO: 5127), PLNGAVDLY (SEQ ID NO: 5128), PLNGAAHLY (SEQ ID NO: 5129), PLNGAVHRY (SEQ ID NO: 5130), PLNGAFHLY (SEQ ID NO: 5131), PLNGADHLY (SEQ ID NO: 5132), PLNGAVLLY (SEQ ID NO: 5133), PLNGAGHLY (SEQ ID NO: 5134), PLNGAVRLY (SEQ ID NO: 5135), PLNGAVHVY (SEQ ID NO: 5136), or PLNGAVYLY (SEQ ID NO: 5137); an amino acid sequence comprising any portion of any of the aforesaid amino acid sequences, e.g., any 2, 3, 4, 5, 6, 7, or 8 amino acids, e.g., consecutive amino acids, thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the amino aforesaid acid sequences.

In some embodiments, a AAV capsid polypeptide, e.g., the AAV capsid variant, comprising an amino acid sequence comprising the formula [A][B], further comprises [C] which comprises X4, X5, X6, and X7. In some embodiments, position X4 of [C] is: A, D, S, or T. In some embodiments, position X5 of [C] is Q, K, H, L, P, or R. In some embodiments, position X6 of [C] is A, P, or E. In some embodiments, position X7 of [C] is Q, H, K, or P. In some embodiments [C] comprises AQ, AK, DQ, SQ, AH, AL, AP, AR, TQ, PQ, EQ, QA, QP, KA, HA, QE, LA, PA, or RA. In some embodiments [C] comprises AQA, AQP, AKA, DQA, SQA, AHA, AQE, ALA, APA, ARA, TQA, QAQ, QPQ, KAQ, HAQ, QEQ, QAK, LAQ, PAQ, RAQ, QAH, or QAP. In some embodiments [C] is or comprises AQAQ (SEQ ID NO: 4737), AQPQ (SEQ ID NO: 4739), AKAQ (SEQ ID NO: 4741), DQAQ (SEQ ID NO: 4744), SQAQ (SEQ ID NO: 4738), AHAQ (SEQ ID NO: 4742), AQEQ (SEQ ID NO: 4748), AQAK (SEQ ID NO: 4746), ALAQ (SEQ ID NO: 4749), APAQ (SEQ ID NO: 4745), ARAQ (SEQ ID NO: 4750), AQAH (SEQ ID NO: 4747), AQAP (SEQ ID NO: 4743), or TQAQ (SEQ ID NO: 4751). In some embodiments, [C] is or comprises AQAQ (SEQ ID NO: 4737), AQPQ (SEQ ID NO: 4739), AKAQ (SEQ ID NO: 4741), DQAQ (SEQ ID NO: 4744), or SQAQ (SEQ ID NO: 4738). In some embodiments [B][C] is or comprises VHLYAQAQ (SEQ ID NO: 4797), VHHYAQAQ (SEQ ID NO: 4804), VHLYAQPQ (SEQ ID NO: 4798), VHLYAKAQ (SEQ ID NO: 4800), VHLYDQAQ (SEQ ID NO: 4801), VHLYSQAQ (SEQ ID NO: 4799), VHIYAQAQ (SEQ ID NO: 4802), VHLYAHAQ (SEQ ID NO: 5138), VNLYAQAQ (SEQ ID NO: 5139), VQLYAQAQ (SEQ ID NO: 5140), VHLYAQEQ (SEQ ID NO: 5141), IHLYAQAQ (SEQ ID NO: 5142), LHLYAQAQ (SEQ ID NO: 5143), VPLYAQAQ (SEQ ID NO: 5144), VHLYAQAK (SEQ ID NO: 5145), VDLYAQAQ (SEQ ID NO: 5146), AHLYAQAQ (SEQ ID NO: 5147), VHRYAQAQ (SEQ ID NO: 5148), FHLYAQAQ (SEQ ID NO: 5149), VHLYALAQ (SEQ ID NO: 5150), DHLYAQAQ (SEQ ID NO: 5151), VHLYAPAQ (SEQ ID NO: 5152), VHLYARAQ (SEQ ID NO: 5153), VHLYAQAH (SEQ ID NO: 5154), VLLYAQAQ (SEQ ID NO: 5155), VHLYAQAP (SEQ ID NO: 5156), GHLYAQAQ (SEQ ID NO: 5157), VRLYAQAQ (SEQ ID NO: 5158), VHVYAQAQ (SEQ ID NO: 4803), VYLYAQAQ (SEQ ID NO: 5159), VHLYTQAQ (SEQ ID NO: 5160); an amino acid sequence comprising any portion of any of the aforesaid amino acid sequences, e.g., any 2, 3, 4, 5, 6, or 7 amino acids, e.g., consecutive amino acids, thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences. In some embodiments, [A][B][C][D] is or comprises PLNGAVHLYAQAQ (SEQ ID NO: 4836), PLNGAVHHYAQAQ (SEQ ID NO: 4850), PLNGAVHLYAQPQ (SEQ ID NO: 4837), PLNGAVHLYAKAQ (SEQ ID NO: 4835), PLNGAVHLYDQAQ (SEQ ID NO: 4838), PLNGAVHLYSQAQ (SEQ ID NO: 4839), PLNGAVHIYAQAQ (SEQ ID NO: 4848), PLNGAVHLYAHAQ (SEQ ID NO: 5181), PLNGAVNLYAQAQ (SEQ ID NO: 5182), PLNGAVQLYAQAQ (SEQ ID NO: 5183), PLNGAVHLYAQEQ (SEQ ID NO: 5184), PLNGAIHLYAQAQ (SEQ ID NO: 5185), PLNGALHLYAQAQ (SEQ ID NO: 5186), PLNGAVPLYAQAQ (SEQ ID NO: 5187), PLNGAVHLYAQAK (SEQ ID NO: 5188), PLNGAVDLYAQAQ (SEQ ID NO: 5189), PLNGAAHLYAQAQ (SEQ ID NO: 5190), PLNGAVHRYAQAQ (SEQ ID NO: 5191), PLNGAFHLYAQAQ (SEQ ID NO: 5192), PLNGAVHLYALAQ (SEQ ID NO: 5193), PLNGADHLYAQAQ (SEQ ID NO: 5194), PLNGAVHLYAPAQ (SEQ ID NO: 5195), PLNGAVHLYARAQ (SEQ ID NO: 5196), PLNGAVHLYAQAH (SEQ ID NO: 5197), PLNGAVLLYAQAQ (SEQ ID NO: 5198), PLNGAVHLYAQAP (SEQ ID NO: 5199), PLNGAGHLYAQAQ (SEQ ID NO: 5200), PLNGAVRLYAQAQ (SEQ ID NO: 5201), PLNGAVHVYAQAQ (SEQ ID NO: 5202), PLNGAVYLYAQAQ (SEQ ID NO: 5203), PLNGAVHLYTQAQ (SEQ ID NO: 5204); an amino acid sequence comprising any portion of any of the aforesaid amino acid sequences, e.g., any 2, 3, 4, 5, 6, 7, 8, 9, 10, 11, or 12 amino acids, e.g., consecutive amino acids, thereof; an amino acid sequence comprising at least one, two, or three but no more than four modifications, e.g., substitutions (e.g., conservative substitutions), insertions, or deletions, relative to any of the aforesaid amino acid sequences; or an amino acid sequence comprising at least one, two, or three but no more than four different amino acids, relative to any one of the aforesaid amino acid sequences.

In some embodiments, the AAV capsid polypeptide, e.g., the AAV capsid variant, comprising an amino acid sequence comprising the formula [A][B], further comprises [D], which comprises X8, X9, and X10. In some embodiments, position X8 of [D] is: T, V, S, L, R, I, A, N, C, Q, M, P, or K. In some embodiments, position X9 of [D] is: T, M, A, G, K, S, Q, V, I, R, N, P, L, H, or Y. In some embodiments, position X10 of [D] is: K, Q, W, S, P, C, A, G, N, T, R, V, M, H, L, E, F, or Y. In some embodiments, [D] comprises TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, PG, QS, RY, SQ, NQ, LL, LP, AQ, TY, NL, SP, LV, KG, VP, AV, KS, AM, SL, AL, RP, IP, MK, AW, GS, KQ, AP, SK, AK, GC, QK, MQ, QP, GP, QQ, AN, GK, QR, PP, AR, GG, MS, NP, KP, MN, KA, SN, MP, HP, GN, RW, MT, SR, GW, QH, GL, QM, VW, MG, AH, QT, GR, SH, GQ, GT, GA, NG, QN, VE, MM, QL, QG, YS, GM, LR, AF, PQ, SW, QW, YA, ML, GF, PA, PS, PT, GY, GV, PW, PR. In some embodiments, [D] is or comprises TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG.

In some embodiments, the AAV capsid polypeptide, e.g., the AAV capsid variant, comprising the amino acid sequence comprising the formula [A][B], further comprises [E], which comprises X11, X12, and X13. In some embodiments, X1I of [E] is: V, D, F, A, E, L, G, or I. In some embodiments, X12 of [E] is Q, R, P, K, L, H, or E. In some embodiments, X13 of [E] is: N, H, S, T, P, K, I, D, or Y. In some embodiments, [E] comprises VQ, DQ, FQ, VR, VP, VK, AQ, EQ, LQ, GQ, VL, VH, VE, DK, GH, IQ, QN, QH, QS, QT, QP, RN, PN, KN, QK, QI, LN, QD, HN, KT, KK, EN, QY, or PH. In some embodiments, [E] is or comprises VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH.

In some embodiments, [A][B][C][D][E] of the AAV capsid variant comprises the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, or 1135; an amino acid sequence comprising any portion of any of the aforesaid amino acid sequences; an amino acid sequence comprising at least one, two, or three but no more than four modifications, e.g., substitutions (e.g., conservative substitutions), insertions, or deletions, relative to any of the aforesaid amino acid sequences; an amino acid sequence comprising at least one, two, or three but no more than four different amino acids, relative to any one of the aforesaid amino acid sequences.

In some embodiments, [A][B] is present in loop VIII of the AAV capsid variant. In some embodiments, [C], [D], and/or [E] is present in loop VIII of the AAV capsid variant. In some embodiments, [A] is present immediately subsequent to position 586, relative to a reference sequence numbered according to the amino acid sequence of SEQ ID NO: 138. In some embodiments, [B] is present immediately subsequent to [A]. In some embodiments, [C] is present immediately subsequent to position 588, relative to a reference sequence numbered according to the amino acid sequence of SEQ ID NO: 138. In some embodiments, [D] is present immediately subsequent to position 592, relative to a reference sequence numbered according to the amino acid sequence of SEQ ID NO: 138. In some embodiments, [E] is present immediately subsequent to position 595, relative to a reference sequence numbered according to the amino acid sequence of SEQ ID NO: 138.

In some embodiments, the AAV capsid polypeptide, e.g., the AAV capsid variant, comprises from N-terminus to C-terminus [A][B]. In some embodiments, the AAV capsid polypeptide, e.g., the AAV capsid variant, comprises from N-terminus to C-terminus [A][B][C]. In some embodiments, the AAV capsid polypeptide, e.g., the AAV capsid variant, comprises from N-terminus to C-terminus [A][B][C][D]. In some embodiments, the AAV capsid polypeptide, e.g., the AAV capsid variant, comprises from N-terminus to C-terminus [A][B][C][D][E].

In some embodiments, the AAV capsid polypeptide, e.g., the AAV capsid variant, comprises comprising PLNGAVHLY (SEQ ID NO: 3648) and optionally further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138. In some embodiments, the amino acid at position 593, relative to a reference sequence numbered according to the amino acid sequence of SEQ ID NO: 138, is: T, A, L, R, V, C, I, K, M, N, P, Q, or S. In some embodiments, the amino acid at position 594, relative to a reference sequence numbered according to the amino acid sequence of SEQ ID NO: 138, is: G, M, S, A, Q, V, T, L, P, H, K, N, I, Y, or R. In some embodiments, the amino acid at position 595, relative to a reference sequence numbered according to the amino acid sequence of SEQ ID NO: 138: W, S, P, T, A, G, L, Q, H, N, R, K, V, E, F, M, C, or Y. In some embodiments, the AAV capsid variant comprises the amino acid sequence of PLNGAVHLY (SEQ ID NO: 3648) and further comprises the amino acid at position 593, relative to a reference sequence numbered according to the amino acid sequence of SEQ ID NO: 138, is: T, A, L, R, V, C, I, K, M, N, P, Q, or S; the amino acid at position 594, relative to a reference sequence numbered according to the amino acid sequence of SEQ ID NO: 138, is: G, M, S, A, Q, V, T, L, P, H, K, N, I, Y, or R; and/or the amino acid at position 595, relative to a reference sequence numbered according to the amino acid sequence of SEQ ID NO: 138: W, S, P, T, A, G, L, Q, H, N, R, K, V, E, F, M, C, or Y. In some embodiments, the amino acids at positions 593-595, relative to a reference sequence numbered according to the amino acid sequence of SEQ ID NO: 138, does not comprise the amino acid sequence of TGW. In some embodiments, the amino acid sequence of PLNGAVHLY (SEQ ID NO: 3648) is present in loop VIII of the AAV capsid variant. In some embodiments, the amino acid sequence of PLNGAVHLY (SEQ ID NO: 3648) is present immediately subsequent to position 586, relative to a reference sequence numbered according to the amino acid sequence of SEQ ID NO: 138.

In some embodiments, the AAV capsid polypeptide, e.g., the AAV capsid variant, comprises at least 3, 4, 5, 6, 7, 8, 9, 10, 11, 12, 13, 14, 15, 16, 17, or 18 consecutive amino acids from any one of SEQ ID NOs: 139-1138. In some embodiments, the AAV capsid polypeptide, e.g., the AAV capsid variant, comprises at least 3, 4, 5, 6, 7, 8, 9, 10, 11, 12, 13, 14, 15, 16, 17, or 18 consecutive amino acids from any one of SEQ ID NOs: 139-476. In some embodiments, the AAV capsid polypeptide, e.g., the AAV capsid variant, comprises at least 3, 4, 5, 6, 7, 8, 9, 10, 11, 12, 13, 14, 15, 16, 17, or 18 consecutive amino acids from any one of the amino acid sequences provided in Table 1B. In some embodiments, the AAV capsid polypeptide, e.g., the AAV capsid variant, comprises at least 3, 4, 5, 6, 7, 8, 9, 10, 11, 12, 13, 14, 15, 16, 17, or 18 consecutive amino acids from any one of the amino acid sequences provided in Table 11. In some embodiments, the AAV capsid polypeptide, e.g., the AAV capsid variant, comprises at least 3, 4, 5, 6, 7, 8, 9, 10, 11, 12, 13, 14, 15, 16, 17, or 18 consecutive amino acids from any one of the amino acid sequences provided in Table 20.

In some embodiments, the AAV capsid variant comprises an amino sequence comprising at least one, two, or three but no more than four modifications, e.g., substitutions (e.g., conservative substitutions), insertions, or deletions, relative to the amino acid sequence of any one of SEQ ID NOs: 139-1138. In some embodiments, the AAV capsid variant comprises an amino sequence comprising at least one, two, or three but no more than four different amino acids, relative to the amino acid sequence of any one of SEQ ID NOs: 139-1138.

In some embodiments, the AAV capsid polypeptide, e.g., the AAV capsid variant, comprises an amino sequence comprising at least one, two, or three but no more than four modifications, e.g., substitutions (e.g., conservative substitutions), insertions, or deletions, relative to the amino acid sequence of any one of SEQ ID NOs: 139-476. In some embodiments, the AAV capsid variant comprises an amino sequence comprising at least one, two, or three but no more than four different amino acids, relative to the amino acid sequence of any one of SEQ ID NOs: 139-476.

In some embodiments, the AAV capsid polypeptide, e.g., the AAV capsid variant, comprises an amino sequence comprising at least one, two, or three but no more than four modifications, e.g., substitutions (e.g., conservative substitutions), insertions, or deletions, relative to any one of the amino acid sequences in Table 1B. In some embodiments, the AAV capsid variant comprises an amino sequence comprising at least one, two, or three but no more than four different amino acids, relative to any one of the amino acid sequences in Table 1B.

In some embodiments, the AAV capsid polypeptide, e.g., the AAV capsid variant, comprises an amino sequence comprising at least one, two, or three but no more than four modifications, e.g., substitutions (e.g., conservative substitutions), insertions, or deletions, relative to the amino acid sequence of any one of SEQ ID NOs: 1139-1172. In some embodiments, the AAV capsid variant comprises an amino sequence comprising at least one, two, or three but no more than four different amino acids, relative to the amino acid sequence of any one of SEQ ID NOs: 1139-1172.

In some embodiments, the amino acid sequence is present in loop VIII of the AAV capsid variant. In some embodiments, the amino acid sequence is present immediately subsequent to position 586, 587, 588, 589, 590, 591, 592, 593, 594, or 595, relative to a reference sequence numbered according to the amino acid sequence of SEQ ID NO: 138. In some embodiments, the amino acid sequence is present immediately subsequent to position 586, relative to a reference sequence numbered according to the amino acid sequence of SEQ ID NO: 138. In some embodiments, the amino acid sequence is present immediately subsequent to position 588, relative to a reference sequence numbered according to the amino acid sequence of SEQ ID NO: 138. In some embodiments, the amino acid sequence is present immediately subsequent to position 592, relative to a reference sequence numbered according to the amino acid sequence of SEQ ID NO: 138. In some embodiments, the amino acid sequence is present immediately subsequent to position 595, relative to a reference sequence numbered according to the amino acid sequence of SEQ ID NO: 138.

In some embodiments, the AAV capsid polypeptide, e.g., the AAV capsid variant, comprises at least 3, 4, 5, 6, 7, 8, or 9 consecutive amino acids from the amino acid sequence of any of SEQ ID NO: 1725-3622. In some embodiments, the AAV capsid variant comprises at least 3, 4, 5, 6, 7, 8, or 9 consecutive amino acids from the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the 3 consecutive amino acids comprise PLN. In some embodiments, the 4 consecutive amino acids comprise PLNG (SEQ ID NO: 3678). In some embodiments, the 5 consecutive amino acids comprise PLNGA (SEQ ID NO: 3679). In some embodiments, the 6 consecutive amino acids comprise PLNGAV (SEQ ID NO: 3680). In some embodiments, the 7 consecutive amino acids comprise PLNGAVH (SEQ ID NO: 3681). In some embodiments, the 8 consecutive amino acids comprise PLNGAVHL (SEQ ID NO: 3682). In some embodiments, the 9 consecutive amino acids comprise PLNGAVHLY (SEQ ID NO: 3648).

In some embodiments, the 3 consecutive amino acids comprise YST. In some embodiments, the 4 consecutive amino acids comprise YSTD (SEQ ID NO: 3690). In some embodiments, the 5 consecutive amino acids comprise YSTDE (SEQ ID NO: 3691). In some embodiments, the 5 consecutive amino acids comprise YSTDV (SEQ ID NO: 3700). In some embodiments, the 6 consecutive amino acids comprise YSTDER (SEQ ID NO: 3692). In some embodiments, the 6 consecutive amino acids comprise YSTDVR (SEQ ID NO: 3701). In some embodiments, the 7 consecutive amino acids comprise YSTDERM (SEQ ID NO: 3657). In some embodiments, the 7 consecutive amino acids comprise YSTDERK (SEQ ID NO: 3658). In some embodiments, the 7 consecutive amino acids comprise YSTDVRM (SEQ ID NO: 3650).

In some embodiments, the 3 consecutive amino acids comprise IVM. In some embodiments, the 4 consecutive amino acids comprise IVMN (SEQ ID NO: 3693). In some embodiments, the 5 consecutive amino acids comprise IVMNS (SEQ ID NO: 3694). In some embodiments, the 6 consecutive amino acids comprise IVMNSL (SEQ ID NO: 3695). In some embodiments, the 7 consecutive amino acids comprise IVMNSLK (SEQ ID NO: 3651).

In some embodiments, the AAV capsid polypeptide, e.g., the AAV capsid variant, comprises an amino acid sequence comprising at least one, two, or three but no more than four modifications, e.g., substitutions, (e.g., conservative substitutions), insertions, or deletions, relative to the amino acid sequence of any of SEQ ID NO: 1725-3622. In some embodiments, the AAV capsid polypeptide, e.g., the AAV capsid variant, comprises an amino acid sequence comprising at least one, two, or three but no more than four different amino acids, relative to the amino acid sequence of any of SEQ ID NO: 1725-3622. In some embodiments, the AAV capsid variant comprises an amino acid sequence comprising at least one, two, or three but no more than four modifications, e.g., substitutions, (e.g., conservative substitutions), insertions, or deletions, relative to the amino acid sequence of any of SEQ ID NO: 3648-3659. In some embodiments, the AAV capsid variant comprises an amino acid sequence comprising at least one, two, or three but no more than four different amino acids relative to the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid polypeptide, e.g., the AAV capsid variant, comprises the amino acid sequence of PLNGAVHLY (SEQ ID NO: 3648), an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648), or an amino acid sequence comprising at least one, two, or three but no more than four different amino acids relative to the amino acid sequence of PLNGAVHLY (SEQ ID NO: 3648), optionally wherein position 7 is H.

In some embodiments, the AAV capsid polypeptide, e.g., the AAV capsid variant, comprises the amino acid sequence of any of SEQ ID NO: 1725-3622. In some embodiments, the AAV capsid variant comprises the amino acid sequence of any of SEQ ID NO: 3648-3659. In some embodiments, the amino acid sequence is present in loop VIII of an AAV capsid variant described herein. In some embodiments, the amino acid sequence is present immediately subsequent to position 586, relative to a reference sequence numbered according to the amino acid sequence of SEQ ID NO: 138. In some embodiments, the amino acid sequence is present immediately subsequent to position 588, relative to a reference sequence numbered according to the amino acid sequence of SEQ ID NO: 138. In some embodiments, the amino acid sequence is present immediately subsequent to position 589, relative to a reference sequence numbered according to the amino acid sequence of SEQ ID NO: 138.

In some embodiments, the AAV capsid polypeptide, e.g., the AAV capsid variant (e.g., an AAV capsid variant described herein), comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the AAV capsid polypeptide, e.g., the AAV capsid variant (e.g., an AAV capsid variant described herein), comprises an amino acid sequence encoded by a nucleotide sequence comprising at least one, two, three, four, five, six, or seven, but no more than ten different nucleotides relative to the nucleotide sequence of any of SEQ ID NOs: 3660-3671.

In some embodiments, the nucleotide sequence encoding the AAV capsid polypeptide, e.g., the AAV capsid variant (e.g., an AAV capsid variant described herein), comprises the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, nucleic acid sequence encoding the AAV capsid variant, e.g., an AAV capsid variant described herein, comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, of the nucleotide sequences relative to any of SEQ ID NOs: 3660-3671. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of any of SEQ ID NOs: 3660-3671.

In some embodiments, the nucleotide sequence encoding the AAV capsid polypeptide, e.g., the AAV capsid variant (e.g., an AAV capsid variant described herein), comprises the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 3660. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of SEQ ID NO: 3660.

In some embodiments, the nucleotide sequence encoding the AAV capsid polypeptide, e.g., the AAV capsid variant (e.g., an AAV capsid variant described herein), comprises the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, of the nucleotide sequences relative to SEQ ID NO: 3663. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of SEQ ID NO: 3663.

In some embodiments, the AAV capsid polypeptide, e.g., the AAV capsid variant, comprises the amino acid P at position 587 and the amino acid L at position 588, and further comprises the amino acid sequence NGAVHLY (SEQ ID NO: 3689) immediately subsequent to position 588, numbered according to SEQ ID NO: 138. In some embodiments, the AAV capsid variant comprises the amino acid L at position 593, the amino acid S at position 594, and/or the amino acid P at position 595, numbered according to SEQ ID NO: 138. In some embodiments, the AAV capsid polypeptide further comprises the amino acid K at position 597, numbered according to SEQ ID NO: 138. In some embodiments, the AAV capsid polypeptide further comprises the amino acid P at position 597, numbered according to SEQ ID NO: 138.

In some embodiments, the AAV capsid polypeptide, e.g., the AAV capsid variant, comprises an amino acid residue other than “A” at position 587 and/or an amino acid residue other than “Q” at position 588, numbered according to SEQ ID NO: 138. In some embodiments, the AAV capsid polypeptide, e.g., the AAV capsid variant, comprises one, two, three, or all of an amino acid other than A at position 589, an amino acid other than Q at position 590, an amino acid other than A at position 591, and/or an amino acid other than Q at position 592, relative to a reference sequence numbered according to the amino acid sequence of SEQ ID NO: 138. In some embodiments, the AAV capsid polypeptide, e.g., the AAV capsid variant, comprises one, two, or all of an amino acid other than T at position 593, an amino acid other than G at position 594, and/or an amino acid other than W at position 595, relative to a reference sequence numbered according to the amino acid sequence of SEQ ID NO: 138. In some embodiments, the AAV capsid polypeptide, e.g., the AAV capsid variant, comprises one, two, or all of an amino acid other than V at position 596, an amino acid other than Q at position 597, and/or an amino acid other than N at position 598, relative to a reference sequence numbered according to the amino acid sequence of SEQ ID NO: 138.

In some embodiments, the AAV capsid polypeptide, e.g., the AAV capsid variant, comprises an amino acid other than T at position 593, an amino acid other than G at position 594, and/or an amino acid other than W at position 595, numbered according to SEQ ID NO: 138. In some embodiments, the AAV capsid variant comprises the amino acid L at position 593, the amino acid S at position 594, and/or the amino acid P at position 595, numbered according to SEQ ID NO: 138.

In some embodiments, the AAV capsid polypeptide, e.g., the AAV capsid variant, comprises an amino acid other than Q at position 597, numbered according to the amino acid sequence of SEQ ID NO: 138. In some embodiments, the AAV capsid variant comprises the amino acid P at position 597, numbered according to the amino acid sequence of SEQ ID NO: 138. In some embodiments, the AAV capsid variant comprises the amino acid K at position 597, numbered according to the amino acid sequence of SEQ ID NO: 138.

In some embodiments, the AAV capsid polypeptide, e.g., the AAV capsid variant, comprises the amino acid sequence of PLNGAVHLY (SEQ ID NO: 3648) wherein the amino acid sequence of PLNGAVHLY (SEQ ID NO: 3648) is present immediately subsequent to position 586, relative to a reference sequence numbered according to the amino acid sequence of SEQ ID NO: 138.

In some embodiments, the polypeptide, e.g., the AAV capsid variant, comprises the amino acid sequence of GGTLAVVSL (SEQ ID NO: 3654), wherein the amino acid sequence of GGTLAVVSL (SEQ ID NO: 3654) is present immediately subsequent to position 586, relative to a reference sequence numbered according to the amino acid sequence of SEQ ID NO: 138.

In some embodiments, the AAV capsid polypeptide, e.g., the AAV capsid variant, comprises the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138.

In some embodiments, the AAV capsid polypeptide, e.g., the AAV capsid variant, comprises the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.

In some embodiments, the AAV capsid polypeptide, e.g., the AAV capsid variant, further comprises a substitution at position K449, e.g., a K449R substitution, numbered according to SEQ ID NO: 138. In some embodiments, the AAV capsid variant further comprises a modification, e.g., an insertion, substitution, and/or deletion in loop I, II, IV, and/or VI.

In some embodiments, the AAV capsid polypeptide, e.g., the AAV capsid variant, further comprises an amino acid sequence comprising at least one, two or three modifications, e.g., substitutions (e.g., conservative substitutions), insertions or deletions, but not more than 30, 20 or 10 modifications relative to the amino acid sequence of SEQ ID NO: 138. In some embodiments, the AAV capsid polypeptide, e.g., the AAV capsid variant, further comprises an amino acid sequence having at least one, two or three but no more than 30, 20, or 10 different amino acids relative to the amino acid sequence of SEQ ID NO: 138. In some embodiments, the AAV capsid variant further comprises the amino acid sequence of SEQ ID NO: 138, or an amino acid sequence with at least 80% (e.g., at least about 85, 90, 95, 96, 97, 98, or 99%) sequence identity thereto. In some embodiments, the AAV capsid variant further comprises an amino acid sequence encoded by the nucleotide sequence of SEQ ID NO: 137, or a sequence with at least 80% (e.g., at least about 85, 90, 95, 96, 97, 98, or 99%) sequence identity thereto.

In some embodiments, an AAV capsid polypeptide, e.g., an AAV capsid variant, of the present disclosure comprises an amino acid sequence as described herein, e.g., an amino acid sequence of an AAV capsid variant chosen from TTD-001, TTD-002, TTD-003, TTD-004, TTD-005, TTD-006, TTD-007, TTD-008, TTD-009, TTD-010, TTD-011, TTD-012, TTD-013, or TTD-014, e.g., as described in Tables 3 and 4.

In some embodiments, an AAV capsid polypeptide, e.g. the AAV capsid variant, comprises a VP1, VP2, and/or VP3 protein comprising an amino acid sequence described herein, e.g., an amino acid sequence of an AAV capsid variant chosen from TTD-001, TTD-002, TTD-003, TTD-004, TTD-005, TTD-006, TTD-007, TTD-008, TTD-009, TTD-010, TTD-011, TTD-012, TTD-013, or TTD-014, e.g., as described in Tables 3 and 4.

In some embodiments, an AAV capsid polypeptide, e.g., the AAV capsid variant, comprises an amino acid sequence encoded by a nucleotide sequence as described herein, e.g., a nucleotide sequence of an AAV capsid variant chosen from TTD-001, TTD-002, TTD-003, TTD-004, TTD-005, TTD-006, TTD-007, TTD-008, TTD-009, TTD-010, TTD-011, TTD-012, TTD-013, or TTD-014, e.g., as described in Tables 3 and 5.

In some embodiments, a polynucleotide encoding an AAV capsid polypeptide, e.g., an AAV capsid variant, of the present disclosure comprises a nucleotide sequence described herein, e.g., a nucleotide sequence of an AAV capsid variant chosen from TTD-001, TTD-002, TTD-003, TTD-004, TTD-005, TTD-006, TTD-007, TTD-008, TTD-009, TTD-010, TTD-011, TTD-012, TTD-013, or TTD-014, e.g., as described in Tables 3 and 5.

TABLE 3
Exemplary full length capsid sequences

| | VP1 DNA | VP1 PRT | Peptide PRT | Peptide DNA |
|---|---|---|---|---|
| Serotype | SEQ ID NO: | SEQ ID NO: | SEQ ID NO: | SEQ ID NO: |
| TTD-001 | 3623 | 3636 | 1725 or 3648 | 3660 |
| TTD-002 | 3624 or 3625 | 3637 | 1726 or 3649 | 3661 |
| TTD-003 | 3626 | 3638 | 1729 or 3650 | 3662 |
| TTD-004 | 3627 | 3639 | 1760 or 3651 | 3663 |
| TTD-005 | 3628 | 3640 | 1769 or 3652 | 3664 |
| TTD-006 | 3629 | 3641 | 3622 or 3653 | 3665 |
| TTD-007 | 3630 | 3642 | 1798 or 3654 | 3666 |
| TTD-008 | 3631 | 3643 | 1785 or 3655 | 3667 |
| TTD-009 | 3632 | 3644 | 1767 or 3656 | 3668 |
| TTD-010 | 3633 | 3645 | 1734 or 3657 | 3669 |
| TTD-011 | 3634 | 3646 | 1737 or 3658 | 3670 |
| TTD-012 | 3635 | 3647 | 1819 or 3659 | 3671 |
| TTD-013 | 4 | 5 | 314 | 6 |
| TTD-014 | 7 | 8 | 566 | 9 |

TABLE 4
Exemplary full length capsid amino acid sequences

| | SEQ | |
|---|---|---|
| Name and | ID | |
| Annotation | NO: | Amino Acid Sequence |
| TTD-001 | 3636 | MAADGYLPDWLEDNLSEGIREWWALKPGAPQPKANQQHQDNARGLVLPG |
| 9 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 587 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 586); | | PRDWQRLINNNWGFRPKRLNFKLFNIQVKEVTDNNGVKTIANNLTSTVQ |
| 743 aa | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSPL |
| | | NGAVHLYAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-002 | 3637 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 589); | | PRDWQRLINNNWGFRPKRLNFKLFNIQVKEVTDNNGVKTIANNLTSTVQ |
| 743 aa | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ARDSPKGWQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-003 | 3638 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 589); | | PRDWQRLINNNWGFRPKRLNFKLFNIQVKEVTDNNGVKTIANNLTSTVQ |
| 743 aa | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYSTDVRMQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-004 | 3639 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 589 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 588); | | PRDWQRLINNNWGFRPKRLNFKLFNIQVKEVTDNNGVKTIANNLTSTVQ |
| 743 aa | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | IVMNSLKAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-005 | 3640 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 589); | | PRDWQRLINNNWGFRPKRLNFKLFNIQVKEVTDNNGVKTIANNLTSTVQ |
| 743 aa | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ARESPRGLQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-006 | 3641 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to 589); | | TWALPTYNNHLYKQISNSTSGGSSNDNAYFGYSTPWGYFDFNRFHCHFS |
| 743 aa | | PRDWQRLINNNWGFRPKRLNFKLFNIQVKEVTDNNGVKTIANNLTSTVQ |
| | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ASFNDTRAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-007 | 3642 | MAADGYLPDWLEDNLSEGIREWWALKPGAPQPKANQQHQDNARGLVLPG |
| 9 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 587 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 586); | | PRDWQRLINNNWGFRPKRLNFKLFNIQVKEVTDNNGVKTIANNLTSTVQ |
| 743 aa | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSGG |
| | | TLAVVSLAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEQYTSNYYKSNNVEFAVNTEGVYSEPRPIG |
| | | TRYLTRNL |
| TTD-008 | 3643 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to 589); | | TWALPTYNNHLYKQISNSTSGGSSNDNAYFGYSTPWGYFDFNRFHCHFS |
| 743 aa | | PRDWQRLINNNWGFRPKRLNFKLFNIQVKEVTDNNGVKTIANNLTSTVQ |
| | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYGLPKGPQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-009 | 3644 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to 589); | | TWALPTYNNHLYKQISNSTSGGSSNDNAYFGYSTPWGYFDFNRFHCHFS |
| 743 aa | | PRDWQRLINNNWGFRPKRLNFKLFNIQVKEVTDNNGVKTIANNLTSTVQ |
| | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ASTGTLRLQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-010 | 3645 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to 589); | | TWALPTYNNHLYKQISNSTSGGSSNDNAYFGYSTPWGYFDFNRFHCHFS |
| 743 aa | | PRDWQRLINNNWGFRPKRLNFKLFNIQVKEVTDNNGVKTIANNLTSTVQ |
| | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYSTDERMQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-011 | 3646 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to 589); | | TWALPTYNNHLYKQISNSTSGGSSNDNAYFGYSTPWGYFDFNRFHCHFS |
| 743 aa | | PRDWQRLINNNWGFRPKRLNFKLFNIQVKEVTDNNGVKTIANNLTSTVQ |
| | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYSTDERKQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-012 | 3647 | MAADGYLPDWLEDNLSEGIREWWALKPGAPQPKANQQHQDNARGLVLPG |
| 7 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 590 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to 589); | | TWALPTYNNHLYKQISNSTSGGSSNDNAYFGYSTPWGYFDFNRFHCHFS |
| 743 aa | | PRDWQRLINNNWGFRPKRLNFKLFNIQVKEVTDNNGVKTIANNLTSTVQ |
| | | VFTDSDYQLPYVLGSAHEGCLPPFPADVFMIPQYGYLTLNDGSQAVGRS |
| | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYVSSVKMQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-013 | 5 | MAADGYLPDWLEDNLSEGIREWWALKPGAPQPKANQQHQDNARGLVLPG |
| 9 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 587 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 586); | | PRDWQRLINNNWGFRPKRLNFKLFNIQVKEVTDNNGVKTIANNLTSTVQ |
| modification at | | VFTDSDYQLPYVLGSAHEGCLPPFPADVFMIPQYGYLTLNDGSQAVGRS |
| position 604; | | SFYCLEYFPSQMLRTGNNFQFSYEFENVPFHSSYAHSQSLDRLMNPLID |
| 743 aa | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSPL |
| | | NGAVHLYAQAQTGWVPNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-014 | 8 | MAADGYLPDWLEDNLSEGIREWWALKPGAPQPKANQQHQDNARGLVLPG |
| 9 mer peptide | | YKYLGPGNGLDKGEPVNAADAAALEHDKAYDQQLKAGDNPYLKYNHADA |
| underlined, starts at | | EFQERLKEDTSFGGNLGRAVFQAKKRLLEPLGLVEEAAKTAPGKKRPVE |
| position 587 | | QSPQEPDSSAGIGKSGAQPAKKRLNFGQTGDTESVPDPQPIGEPPAAPS |
| (immediately | | GVGSLTMASGGGAPVADNNEGADGVGSSSGNWHCDSQWLGDRVITTSTR |
| subsequent to | | TWALPTYNNHLYKQISNSTSGGSSNDNAYFGYSTPWGYFDFNRFHCHFS |
| position 586); | | PRDWQRLINNNWGFRPKRLNFKLFNIQVKEVTDNNGVKTIANNLTSTVQ |
| modifications at | | VFTDSDYQLPYVLGSAHEGCLPPFPADVFMIPQYGYLTLNDGSQAVGRS |
| positions 600, 601, | | SFYCLEYFPSQMLRTGNNFQFSYEFENVPFHSSYAHSQSLDRLMNPLID |
| 602, and 604; | | QYLYYLSKTINGSGQNQQTLKFSVAGPSNMAVQGRNYIPGPSYRQQRVS |
| 743 aa | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSPL |
| | | NGAVHLYAQAQLSPVKNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |

TABLE 5
Exemplary full length capsid nucleic acid sequences

| | SEQ | |
|---|---|---|
| Name and | ID | |
| Annotation | NO: | NT Sequence |
| TTD-001 | 3623 | atggctgccgatggttatcttccagattggctcgaggacaaccttagtgaaggaattcgcga |
| 9 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtccgcttaatggtgccgtccatctttatgctcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-002 | 3624 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggctcgtgattctccgaagggttggcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| | 3625 | atggctgccgatggttatcttccagattggctcgaggacaaccttagtgaaggaattcgcga |
| | | gtggtgggctttgaaacctggagcccctcaacccaaggcaaatcaacaacatcaagacaacg |
| | | ctcgaggtcttgtgcttccgggttacaaataccttggacccggcaacggactcgacaagggg |
| | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggctcgtgattctccgaagggttggcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcggatcctccaacggccttcaacaaggacaagctgaactctttcatcacccagtattct |
| | | actggccaagtcagcgtggagatcgagtgggagctgcagaaggaaaacagcaagcgctggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-003 | 3626 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggcttattctacggatgtgaggatgcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-004 | 3627 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacagattgttatgaattcgttgaaggctcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-005 | 3628 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggctcgggagagtcctcgtgggctgcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-006 | 3629 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggctagttttaatgatactagggctcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-007 | 3630 | atggctgccgatggttatcttccagattggctcgaggacaaccttagtgaaggaattcgcga |
| 9 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtggtggtacgttggccgtcgtgtcgcttgctcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-008 | 3631 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggcttatgggttgccgaagggtcctcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-009 | 3632 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggcttcgactgggacgcttcggcttcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-010 | 3633 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcgcaggcgtattcgacggatgagaggatgcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-011 | 3634 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcgcaggcgtattcgacggatgagaggaagcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-012 | 3635 | atggctgccgatggttatcttccagattggctcgaggacaaccttagtgaaggaattcgcga |
| 7 mer | | gtggtgggctttgaaacctggagcccctcaacccaaggcaaatcaacaacatcaagacaacg |
| peptide | | ctcgaggtcttgtgcttccgggttacaaataccttggacccggcaacggactcgacaagggg |
| underlined | | gagccggtcaacgcagcagacgcggcggccctcgagcacgacaaggcctacgaccagcagct |
| | | caaggccggagacaacccgtacctcaagtacaaccacgccgacgccgagttccaggagcggc |
| | | tcaaagaagatacgtcttttgggggcaacctcgggcgagcagtcttccaggccaaaaagagg |
| | | cttcttgaacctcttggtctggttgaggaagcggctaagacggctcctggaaagaagaggcc |
| | | tgtagagcagtctcctcaggaaccggactcctccgcgggtattggcaaatcgggtgcacagc |
| | | ccgctaaaaagagactcaatttcggtcagactggcgacacagagtcagtcccagaccctcaa |
| | | ccaatcggagaacctcccgcagccccctcaggtgtgggatctcttacaatggcttcaggtgg |
| | | tggcgcaccagtggcagacaataacgaaggtgccgatggagtgggtagttcctcgggaaatt |
| | | ggcattgcgattcccaatggctgggggacagagtcatcaccaccagcacccgaacctgggcc |
| | | ctgcccacctacaacaatcacctctacaagcaaatctccaacagcacatctggaggatcttc |
| | | aaatgacaacgcctacttcggctacagcaccccctgggggtattttgacttcaacagattcc |
| | | actgccacttctcaccacgtgactggcagcgactcatcaacaacaactggggattccggcct |
| | | aagcgactcaacttcaagctcttcaacattcaggtcaaagaggttacggacaacaatggagt |
| | | caagaccatcgccaataaccttaccagcacggtccaggtcttcacggactcagactatcagc |
| | | tcccgtacgtgctcgggtcggctcacgagggctgcctcccgccgttcccagcggacgttttc |
| | | atgattcctcagtacgggtatctgacgcttaatgatggaagccaggccgtgggtcgttcgtc |
| | | cttttactgcctggaatatttcccgtcgcaaatgctaagaacgggtaacaacttccagttca |
| | | gctacgagtttgagaacgtacctttccatagcagctacgctcacagccaaagcctggaccga |
| | | ctaatgaatccactcatcgaccaatacttgtactatctctcaaagactattaacggttctgg |
| | | acagaatcaacaaacgctaaaattcagtgtggccggacccagcaacatggctgtccagggaa |
| | | gaaactacatacctggacccagctaccgacaacaacgtgtctcaaccactgtgactcaaaac |
| | | aacaacagcgaatttgcttggcctggagcttcttcttgggctctcaatggacgtaatagctt |
| | | gatgaatcctggacctgctatggccagccacaaagaaggagaggaccgtttctttcctttgt |
| | | ctggatctttaatttttggcaaacaaggaactggaagagacaacgtggatgcggacaaagtc |
| | | atgataaccaacgaagaagaaattaaaactactaacccggtagcaacggagtcctatggaca |
| | | agtggccacaaaccaccagagtgcacaggcttatgtttcgtctgttaagatgcaggcgcaga |
| | | ccggctgggttcaaaaccaaggaatacttccgggtatggtttggcaggacagagatgtgtac |
| | | ctgcaaggacccatttgggccaaaattcctcacacggacggcaactttcacccttctccgct |
| | | gatgggagggtttggaatgaagcacccgcctcctcagatcctcatcaaaaacacacctgtac |
| | | ctgcCgatcctccaacggccttcaacaaggacaagctgaactctttcatcacccagtattct |
| | | actggccaagtcagcgtggagatcgagtgggagctgcagaaggaaaacagcaagcgGtggaa |
| | | cccggagatccagtacacttccaactattacaagtctaataatgttgaatttgctgttaata |
| | | ctgaaggtgtatatagtgaaccccgccccattggcaccagatacctgactcgtaatctgtaa |
| TTD-013 | 4 | ATGGCTGCCGATGGTTATCTTCCAGATTGGCTCGAGGACAACCTTAGTGAAGGAATTCGCGA |
| | | GTGGTGGGCTTTGAAACCTGGAGCCCCTCAACCCAAGGCAAATCAACAACATCAAGACAACG |
| | | CTCGAGGTCTTGTGCTTCCGGGTTACAAATACCTTGGACCCGGCAACGGACTCGACAAGGGG |
| | | GAGCCGGTCAACGCAGCAGACGCGGCGGCCCTCGAGCACGACAAGGCCTACGACCAGCAGCT |
| | | CAAGGCCGGAGACAACCCGTACCTCAAGTACAACCACGCCGACGCCGAGTTCCAGGAGCGGC |
| | | TCAAAGAAGATACGTCTTTTGGGGGCAACCTCGGGCGAGCAGTCTTCCAGGCCAAAAAGAGG |
| | | CTTCTTGAACCTCTTGGTCTGGTTGAGGAAGCGGCTAAGACGGCTCCTGGAAAGAAGAGGCC |
| | | TGTAGAGCAGTCTCCTCAGGAACCGGACTCCTCCGCGGGTATTGGCAAATCGGGTGCACAGC |
| | | CCGCTAAAAAGAGACTCAATTTCGGTCAGACTGGCGACACAGAGTCAGTCCCAGACCCTCAA |
| | | CCAATCGGAGAACCTCCCGCAGCCCCCTCAGGTGTGGGATCTCTTACAATGGCTTCAGGTGG |
| | | TGGCGCACCAGTGGCAGACAATAACGAAGGTGCCGATGGAGTGGGTAGTTCCTCGGGAAATT |
| | | GGCATTGCGATTCCCAATGGCTGGGGGACAGAGTCATCACCACCAGCACCCGAACCTGGGCC |
| | | CTGCCCACCTACAACAATCACCTCTACAAGCAAATCTCCAACAGCACATCTGGAGGATCTTC |
| | | AAATGACAACGCCTACTTCGGCTACAGCACCCCCTGGGGGTATTTTGACTTCAACAGATTCC |
| | | ACTGCCACTTCTCACCACGTGACTGGCAGCGACTCATCAACAACAACTGGGGATTCCGGCCT |
| | | AAGCGACTCAACTTCAAGCTCTTCAACATTCAGGTCAAAGAGGTTACGGACAACAATGGAGT |
| | | CAAGACCATCGCCAATAACCTTACCAGCACGGTCCAGGTCTTCACGGACTCAGACTATCAGC |
| | | TCCCGTACGTGCTCGGGTCGGCTCACGAGGGCTGCCTCCCGCCGTTCCCAGCGGACGTTTTC |
| | | ATGATTCCTCAGTACGGGTATCTGACGCTTAATGATGGAAGCCAGGCCGTGGGTCGTTCGTC |
| | | CTTTTACTGCCTGGAATATTTCCCGTCGCAAATGCTAAGAACGGGTAACAACTTCCAGTTCA |
| | | GCTACGAGTTTGAGAACGTACCTTTCCATAGCAGCTACGCTCACAGCCAAAGCCTGGACCGA |
| | | CTAATGAATCCACTCATCGACCAATACTTGTACTATCTCTCAAAGACTATTAACGGTTCTGG |
| | | ACAGAATCAACAAACGCTAAAATTCAGTGTGGCCGGACCCAGCAACATGGCTGTCCAGGGAA |
| | | GAAACTACATACCTGGACCCAGCTACCGACAACAACGTGTCTCAACCACTGTGACTCAAAAC |
| | | AACAACAGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTCCTTTGT |
| | | CTGGATCTTTAATTTTTGGCAAACAAGGAACTGGAAGAGACAACGTGGATGCGGACAAAGTC |
| | | ATGATAACCAACGAAGAAGAAATTAAAACTACTAACCCGGTAGCAACGGAGTCCTATGGACA |
| | | AGTGGCCACAAACCACCAGAGTccgcttaatggtgccgtccatctttatGCTCAGGCGCAGA |
| | | CCGGCTGGGTTccgAACCAAGGAATACTTCCGGGTATGGTTTGGCAGGACAGAGATGTGTAC |
| | | CTGCAAGGACCCATTTGGGCCAAAATTCCTCACACGGACGGCAACTTTCACCCTTCTCCGCT |
| | | GATGGGAGGGTTTGGAATGAAGCACCCGCCTCCTCAGATCCTCATCAAAAACACACCTGTAC |
| | | CTGCCGATCCTCCAACGGCCTTCAACAAGGACAAGCTGAACTCTTTCATCACCCAGTATTCT |
| | | ACTGGCCAAGTCAGCGTGGAGATCGAGTGGGAGCTGCAGAAGGAAAACAGCAAGCGGTGGAA |
| | | CCCGGAGATCCAGTACACTTCCAACTATTACAAGTCTAATAATGTTGAATTTGCTGTTAATA |
| | | CTGAAGGTGTATATAGTGAACCCCGCCCCATTGGCACCAGATACCTGACTCGTAATCTGTAA |
| TTD-014 | 7 | ATGGCTGCCGATGGTTATCTTCCAGATTGGCTCGAGGACAACCTTAGTGAAGGAATTCGCGA |
| | | GTGGTGGGCTTTGAAACCTGGAGCCCCTCAACCCAAGGCAAATCAACAACATCAAGACAACG |
| | | CTCGAGGTCTTGTGCTTCCGGGTTACAAATACCTTGGACCCGGCAACGGACTCGACAAGGGG |
| | | GAGCCGGTCAACGCAGCAGACGCGGCGGCCCTCGAGCACGACAAGGCCTACGACCAGCAGCT |
| | | CAAGGCCGGAGACAACCCGTACCTCAAGTACAACCACGCCGACGCCGAGTTCCAGGAGCGGC |
| | | TCAAAGAAGATACGTCTTTTGGGGGCAACCTCGGGCGAGCAGTCTTCCAGGCCAAAAAGAGG |
| | | CTTCTTGAACCTCTTGGTCTGGTTGAGGAAGCGGCTAAGACGGCTCCTGGAAAGAAGAGGCC |
| | | TGTAGAGCAGTCTCCTCAGGAACCGGACTCCTCCGCGGGTATTGGCAAATCGGGTGCACAGC |
| | | CCGCTAAAAAGAGACTCAATTTCGGTCAGACTGGCGACACAGAGTCAGTCCCAGACCCTCAA |
| | | CCAATCGGAGAACCTCCCGCAGCCCCCTCAGGTGTGGGATCTCTTACAATGGCTTCAGGTGG |
| | | TGGCGCACCAGTGGCAGACAATAACGAAGGTGCCGATGGAGTGGGTAGTTCCTCGGGAAATT |
| | | GGCATTGCGATTCCCAATGGCTGGGGGACAGAGTCATCACCACCAGCACCCGAACCTGGGCC |
| | | CTGCCCACCTACAACAATCACCTCTACAAGCAAATCTCCAACAGCACATCTGGAGGATCTTC |
| | | AAATGACAACGCCTACTTCGGCTACAGCACCCCCTGGGGGTATTTTGACTTCAACAGATTCC |
| | | ACTGCCACTTCTCACCACGTGACTGGCAGCGACTCATCAACAACAACTGGGGATTCCGGCCT |
| | | AAGCGACTCAACTTCAAGCTCTTCAACATTCAGGTCAAAGAGGTTACGGACAACAATGGAGT |
| | | CAAGACCATCGCCAATAACCTTACCAGCACGGTCCAGGTCTTCACGGACTCAGACTATCAGC |
| | | TCCCGTACGTGCTCGGGTCGGCTCACGAGGGCTGCCTCCCGCCGTTCCCAGCGGACGTTTTC |
| | | ATGATTCCTCAGTACGGGTATCTGACGCTTAATGATGGAAGCCAGGCCGTGGGTCGTTCGTC |
| | | CTTTTACTGCCTGGAATATTTCCCGTCGCAAATGCTAAGAACGGGTAACAACTTCCAGTTCA |
| | | GCTACGAGTTTGAGAACGTACCTTTCCATAGCAGCTACGCTCACAGCCAAAGCCTGGACCGA |
| | | CTAATGAATCCACTCATCGACCAATACTTGTACTATCTCTCAAAGACTATTAACGGTTCTGG |
| | | ACAGAATCAACAAACGCTAAAATTCAGTGTGGCCGGACCCAGCAACATGGCTGTCCAGGGAA |
| | | GAAACTACATACCTGGACCCAGCTACCGACAACAACGTGTCTCAACCACTGTGACTCAAAAC |
| | | AACAACAGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTCCTTTGT |
| | | CTGGATCTTTAATTTTTGGCAAACAAGGAACTGGAAGAGACAACGTGGATGCGGACAAAGTC |
| | | ATGATAACCAACGAAGAAGAAATTAAAACTACTAACCCGGTAGCAACGGAGTCCTATGGACA |
| | | gGTcGCtACGAATCATCAGTCTCCGCTGAATGGTGCGGTGCATCTGTATGCGCAGGCGCAGC |
| | | TGTCTCCGGTGAAGAATcaaggaatacttccgggtatgGTTTGGCAGGACAGAGATGTGTAC |
| | | CTGCAAGGACCCATTTGGGCCAAAATTCCTCACACGGACGGCAACTTTCACCCTTCTCCGCT |
| | | GATGGGAGGGTTTGGAATGAAGCACCCGCCTCCTCAGATCCTCATCAAAAACACACCTGTAC |
| | | CTGCCGATCCTCCAACGGCCTTCAACAAGGACAAGCTGAACTCTTTCATCACCCAGTATTCT |
| | | ACTGGCCAAGTCAGCGTGGAGATCGAGTGGGAGCTGCAGAAGGAAAACAGCAAGCGGTGGAA |
| | | CCCGGAGATCCAGTACACTTCCAACTATTACAAGTCTAATAATGTTGAATTTGCTGTTAATA |
| | | CTGAAGGTGTATATAGTGAACCCCGCCCCATTGGCACCAGATACCTGACTCGTAATCTGTAA |

In some embodiments, the polynucleotide encoding an AAV capsid polypeptide, e.g., AAV capsid variant, described herein comprises the nucleotide sequence of any one of SEQ ID NOs: 4, 7, 3623-3635, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3623, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 4, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 7, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3627, or a nucleotide sequence with at least 80% (e.g., at least about 85, 90, 95, 96, 97, 98, or 99%) sequence identity thereto. In some embodiments, the nucleic acid sequence encoding an AAV capsid polypeptide, e.g., an AAV capsid variant, described herein is codon optimized.

In some embodiments, the AAV capsid polypeptide, e.g., the AAV capsid variant, comprises a VP2 protein comprising the amino acid sequence corresponding to positions 138-743, of any one of SEQ ID NOs: 5, 8, 3636-3647, or a sequence with at least 80% (e.g., at least about 85, 90, 95, 96, 97, 98, or 99%) sequence identity thereto. In some embodiments, the AAV capsid comprises a VP3 protein comprising the amino acid sequence corresponding to positions 203-743, of any one of SEQ ID NOs: 5, 8, 3636-3647, or a sequence with at least 80% (e.g., at least about 85, 90, 95, 96, 97, 98, or 99%) sequence identity thereto.

In some embodiments, the AAV capsid polypeptide, e.g., the AAV capsid variant, e.g., an AAV capsid variant described herein, comprises the amino acid sequence of any one of SEQ ID NOs: 5, 8, 3636-3647, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of any one of SEQ ID NOs: 5, 8, 3636-3647. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of any one of SEQ ID NOs: 5, 8, 3636-3647.

In some embodiments, the AAV capsid polypeptide, e.g., the AAV capsid variant, e.g., an AAV capsid variant described herein, comprises the amino acid sequence of SEQ ID NO: 3636, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 3636. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 3636.

In some embodiments, the AAV capsid polypeptide, e.g., the AAV capsid variant, e.g., an AAV capsid variant described herein, comprises the amino acid sequence of SEQ ID NO: 5, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 5. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 5.

In some embodiments, the AAV capsid polypeptide, e.g., the AAV capsid variant, e.g., an AAV capsid variant described herein, comprises the amino acid sequence of SEQ ID NO: 8, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 8. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 8.

In some embodiments, an AAV capsid polypeptide, e.g., an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid polypeptide, e.g., an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 3636.

In some embodiments, an AAV capsid polypeptide, e.g., an AAV capsid variant, described herein transduces a brain region, e.g., selected from dentate nucleus, cerebellar cortex, cerebral cortex, brain stem, hippocampus, thalamus and putamen. In some embodiments, the level of transduction of said brain region is at least 5, 10, 50, 100, 200, 500, 1,000, 2,000, 5,000, or 10,000-fold greater as compared to a reference sequence of SEQ ID NO: 138.

In some embodiments, an AAV capsid polypeptide, e.g., an AAV capsid variant, described herein is enriched at least about 5, 6, 7, 8, 9, or 10-fold, in the brain compared to a reference sequence of SEQ ID NO: 138. In some embodiments, an AAV capsid variant described herein is enriched at least about 20, 30, 40, or 50-fold in the brain compared to a reference sequence of SEQ ID NO: 138. In some embodiments, an AAV capsid variant described herein is enriched at least about 100, 200, 300, or 400-fold in the brain compared to a reference sequence of SEQ ID NO: 138.

In some embodiments, AAV capsid polypeptide, e.g., an AAV capsid variant, described herein is enriched at least about 2, 2.5, 3, 3.5, 4, 4.5, 5, 5.5, or 6-fold, in the brain compared to a reference sequence of SEQ ID NO: 3636. In some embodiments, AAV capsid polypeptide, e.g., an AAV capsid variant, described herein is enriched at least about 1.1, 1.2, 1.3, 1.4, 1.5, 1.6, 1.7, 1.8, 1.9, 2, 2.5, 3, 3.5, 4, 4.5, 5, 5.5, or 6-fold, in the brain compared to a reference sequence of SEQ ID NO: 3636.

In some embodiments, an AAV capsid polypeptide, e.g., an AAV capsid variant, described herein delivers an increased level of viral genomes to a brain region. In some embodiments, the level of viral genomes is increased by at least 5, 10, 20, 30, 40 or 50-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid polypeptide, e.g., an AAV capsid variant, described herein delivers an increased level of a payload to a brain region. In some embodiments, the level of the payload is increased by at least 5, 10, 50, 100, 200, 500, 1,000, 2,000, 5,000, or 10,000-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid polypeptide, e.g., an AAV capsid variant, described herein delivers an increased level of a payload to a spinal cord region. In some embodiments, the level of the payload is increased by at least 10, 20, 50, 100, 200, 300, 400, 500, 600, 700, 800 or 900-fold, as compared to a reference sequence of SEQ ID NO: 138. In some embodiments, the spinal cord region comprises a cervical, thoracic, and/or lumbar region.

In some embodiments, an AAV capsid polypeptide, e.g., an AAV capsid variant, described herein shows preferential transduction in a brain region relative to the transduction in the dorsal root ganglia (DRG).

In some embodiments, an AAV capsid polypeptide, e.g., an AAV capsid variant, described herein has an increased tropism for a muscle cell or tissue, e.g., a heart cell or tissue, relative to the tropism of a reference sequence comprising the amino acid sequence of SEQ ID NO: 138. In some embodiments, the AAV capsid variant delivers an increased level of a payload to a muscle region. In some embodiments, the payload is increased by at least 10, 15, 20, 30, or 40-fold, as compared to a reference sequence of SEQ ID NO: 138. In some embodiments, the muscle region comprises a heart muscle, quadriceps muscle, and/or a diaphragm muscle region. In some embodiments, the muscle region comprises a heart muscle region, e.g., a heart atrium muscle region or a heart ventricle muscle region.

In some embodiments, an AAV capsid polypeptide, e.g., an AAV capsid variant described herein results in greater than 1, 2, 5, 10, 20, 30, 40, 50, or 100 reads per sample, e.g., when analyzed by an NGS sequencing assay.

In some embodiments, an AAV capsid polypeptide, e.g., an AAV capsid variant, of the present disclosure has decreased tropism for the liver. In some embodiments, an AAV capsid variant comprises a modification, e.g., substitution (e.g., conservative substitution), insertion, or deletion, that results in reduced tropism (e.g., de-targeting) and/or activity in the liver. In some embodiments, the reduced tropism in the liver is compared to an otherwise similar capsid that does not comprise the modification, e.g., a wild-type capsid polypeptide. In some embodiments, an AAV capsid variant described comprises a modification, e.g., substitution (e.g., conservative substitution), insertion, or deletion, that results in one or more of the following properties: (1) reduced tropism in the liver; (2) de-targeted expression in the liver; (3) reduced activity in the liver; and/or (4) reduced binding to galactose. In some embodiments, the reduction in any one, or all of properties (1)-(3) is compared to an otherwise similar AAV capsid variant that does not comprise the modification. Exemplary modifications are provided in WO 2018/119330; Pulicherla et al. (2011) Mol. Ther. 19(6): 1070-1078; Adachi et al. (2014) Nature Communications 5(3075), DOI: 10.1038/ncomms4075; and Bell et al. (2012) J. Virol. 86(13): 7326-33; the contents of which are hereby incorporated by reference in their entirety. In some embodiments, the AAV capsid variant comprises a modification e.g., substitution (e.g., conservative substitution), insertion, or deletion, at position N470 (e.g., N470A), D271 (e.g., D271A), N272 (e.g., N297A), Y446 (e.g., Y446A), N498 (e.g., N498Y or N498I), W503 (e.g., W530R or W530A), L620 (e.g., L620F), or a combination thereof, relative to a reference sequence numbered according to SEQ ID NO: 138. In some embodiments, the AAV capsid variant comprises one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498/(e.g., Y or I), and amino acid other than W at position 503 (e.g., R or A), and amino acid other than L at position 620 (e.g., F), relative to a reference sequence numbered according to SEQ ID NO: 138. In some embodiments, the AAV capsid variant comprises a modification e.g., substitution (e.g., conservative substitution), insertion, or deletion, at position N470 (e.g., N470A), D271 (e.g., D271A), N272 (e.g., N297A), Y446 (e.g., Y446A), and W503 (e.g., W530R or W530A), relative to a reference sequence numbered according to SEQ ID NO: 138. In some embodiments, the AAV capsid variant comprises a modification e.g., substitution (e.g., conservative substitution), insertion, or deletion, at N498 (e.g., N498Y) and L620 (e.g., L620F).

In some embodiments, an AAV capsid variant comprised herein comprises a modification as described in Adachi et al. (2014) Nature Communications 5(3075), DOI: 10.1038/ncomms4075, the contents of which are hereby incorporated by reference in its entirety. Exemplary modifications that alter or do not alter tissue transduction in at least the brain, liver, heart, lung, and/or kidney can be found in Supplementary Data 2 showing the AAV Barcode-Seq data obtained with AAV9-AA-VBCLib of Adachi et al. (supra), the contents of which are hereby incorporated by reference in its entirety.

In some embodiments, an, AAV capsid polypeptide, e.g., an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

Also provided herein are polynucleotide sequences encoding any of the AAV capsid variants described above and AAV particles, vectors, cells, and formulations, e.g., pharmaceutical formulations, comprising the same.

### AAV Serotypes and Capsids

In some embodiments, an AAV particle of the present disclosure may comprise a capsid polypeptide or variant thereof from any natural or recombinant AAV serotype. AAV serotypes may differ in characteristics such as, but not limited to, packaging, tropism, transduction and immunogenic profiles. While not wishing to be bound by theory, it is believed in some embodiments, that the AAV capsid protein, e.g., an AAV capsid variant, can modulate, e.g., direct, AAV particle tropism to a particular tissue.

In some embodiments, an AAV capsid polypeptide, e.g., AAV capsid variant, described herein allows for blood brain barrier penetration following intravenous administration. In some embodiments, the AAV capsid polypeptide, e.g., AAV capsid variant, allows for blood brain barrier penetration following focused ultrasound (FUS), e.g., coupled with the intravenous administration of microbubbles (FUS-MB), or MRI-guided FUS coupled with intravenous administration. In some embodiments the AAV capsid polypeptide, e.g., AAV capsid variant allows for increased distribution to a brain region. In some embodiments, the brain region comprises a frontal cortex, sensory cortex, motor cortex, caudate, dentate nucleus, cerebellar cortex, cerebral cortex, brain stem, hippocampus, thalamus, putamen, or a combination thereof. In some embodiments, the AAV capsid polypeptide, e.g., AAV capsid variant allows for preferential transduction in a brain region relative to the transduction in the dorsal root ganglia (DRG).

In some embodiments an AAV capsid polypeptide, e.g., AAV capsid variant, described herein allows for increased distribution to a spinal cord region. In some embodiments, the spinal region comprises a cervical spinal cord region, thoracic spinal cord region, and/or lumbar spinal cord region.

In some embodiments, the AAV capsid polypeptide, e.g., AAV capsid variant, is suitable for intramuscular administration and/or transduction of muscle fibers. In some embodiments the AAV capsid polypeptide, e.g., AAV capsid variant, allows for increased distribution to a muscle region. In some embodiments, the muscle region comprises a heart muscle, quadriceps muscle, a diaphragm muscle region, or a combination thereof. In some embodiments, the muscle region comprises a heart muscle region, e.g., a heart atrium muscle region or a heart ventricle muscle region.

In some embodiments, the initiation codon for translation of the AAV VP1 capsid protein, e.g., a capsid variant, described herein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (e.g. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins, e.g., AAV capsid variants, is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a capsid protein or capsid polypeptide (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Additional AAV Sequences

In some embodiments, the AAV capsid polypeptide, e.g., AAV capsid variant, comprises, immediately subsequent to position 586, 588, or 589 numbered relative to SEQ ID NO: 138, at least 3, 4, 5, 6, 7, 8, 9, 10, 11, 12, 13, 14, 15, 16, 17, or 18 consecutive amino acids of any of SEQ ID NOs: 139-1172, 1725-3622 or 3648-3659.

In some embodiments, the AAV capsid variant, comprises immediately subsequent to position 586, 588, or 589, numbered relative to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety)), at least 3, 4, 5, 6, 7, 8, 9, 10, 11, 12, 13, 14, 15, 16, 17, 18, or 19 consecutive amino acids of any of amino acid sequence provided in Tables 1A, 1B, 2, 7, 10, 11, or 20. In some embodiments, the at least 3, 4, 5, 6, 7, 8, 9, 10, 11, 12, 13, 14, 15, 16, 17, 18, or 19 consecutive amino acids of any of amino acid sequence provided in Tables 1A, 1B, 2, 7, 10, 11, or 20 replaces at least one, two, three, four, five, six, seven, eight, nine, ten, elven, or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the at least 3, 4, 5, 6, 7, 8, 9, 10, 11, 12, 13, 14, 15, 16, 17, 18, or 19 consecutive amino acids of any of amino acid sequence provided in Tables 1A, 1B, 2, 7, 10, 11, or 20 replaces positions A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at one, two, three, four, five, six, seven, eight, nine, ten, eleven or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at position A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety)). In some embodiments, the AAV capsid variant comprises a modification, e.g., substitution, at one, two, three, four, five, six, seven, eight, nine, ten eleven or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises a modification, e.g., substitution, at position A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety).

In any of the embodiments described herein, a position comprising 5 consecutive amino acids corresponding to positions 586 to 599, e.g., 586 to 594, 587 to 595, 588 to 596, 589 to 597, 590 to 598 numbered relative to SEQ ID NO: 138 can be identified by providing an alignment of a reference sequence and a query sequence, wherein the reference sequence is SEQ ID NO: 138, and identifying the residues corresponding to the positions in the query sequence that correspond to positions 586 to 599, e.g., 586 to 594, 587 to 595, 588 to 596, 589 to 597, 590 to 598 in the reference sequence.

In some embodiments, the AAV capsid polypeptide, e.g., AAV capsid variant, described herein does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, of any of the amino acid sequences in Table 1 of WO2020223276, the contents of which are hereby incorporated by reference in their entirety.

In some embodiments, the AAV capsid polypeptide, e.g., AAV capsid variant, described herein does not comprise an amino acid sequence present immediately subsequent to position 586, 588, or 589 numbered relative to SEQ ID NO: 138, at least 5 consecutive amino acids corresponding to positions 586 to 599, e.g., 586 to 594, 587 to 595, 588 to 596, 589 to 597, 590 to 598, of SEQ ID NO: 138. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, of SEQ ID NO: 12. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least than 5 consecutive amino acids corresponding to positions 586 to 599, e.g., 586 to 594, 587 to 595, 588 to 596, 589 to 597, 590 to 598, of SEQ ID NO: 13. In some embodiments, the AAV capsid polypeptide, e.g., AAV capsid variant, or the parent AAV capsid may be, at a position other than 5 consecutive amino acids corresponding to positions 586 to 599, e.g., 586 to 594, 587 to 595, 588 to 596, 589 to 597, 590 to 598, of SEQ ID NO: 1. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, of SEQ ID NO: 3.

In some embodiments, the AAV capsid polypeptide, e.g., AAV capsid variant, described herein, does not comprise the amino acid sequence of TLAVPFK (SEQ ID NO: 1262) present immediately subsequent to position 588, numbered according to SEQ ID NO: 138.

In some embodiments, an AAV capsid polypeptide or AAV capsid variant described herein may comprise a VOY101 capsid polypeptide, an AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAV9 K449R capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 6, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide comprises any one of the nucleotide sequences in Table 6, or a nucleotide sequence substantially identical (e.g., having at least 70%, 75%, 80%, 85%, 90%, 92%, 95%, 97%, 98%, or 99% sequence identity) thereto.

In some embodiments, an AAV capsid polypeptide or an AAV capsid variant described herein comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide or the AAV capsid variant, comprises an amino acid sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the amino acid sequence of SEQ ID NO: 138. In some embodiments, the AAV capsid polypeptide or the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide or the AAV capsid variant comprises the nucleotide sequence of SEQ ID NO: 137, or a nucleotide sequence substantially identical (e.g., having at least 70%, 75%, 80%, 85%, 90%, 92%, 95%, 97%, 98%, or 99% sequence identity) thereto. In some embodiments, the AAV capsid polypeptide or the AAV capsid variant, comprises substitution at position K449, e.g., a K449R substitution, numbered according to SEQ ID NO: 138.

In some embodiments, the AAV capsid polypeptide or the AAV capsid variant comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide or the AAV capsid variant, comprises an amino acid sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the amino acid sequence of SEQ ID NO: 11, optionally wherein position 449 is not R.

TABLE 6
AAV Sequences

| | SEQ | |
|---|---|---|
| | ID | |
| Serotype | NO: | Sequence |
| VOY101 | 1 | MAADGYLPDWLEDNLSEGIREWWALKPGAPQPKANQQHQDNARGLVLPGYKYLGPGNGLDK |
| | | GEPVNAADAAALEHDKAYDQQLKAGDNPYLKYNHADAEFQERLKEDTSFGGNLGRAVFQAK |
| | | KRLLEPLGLVEEAAKTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWHCDSQWLGDRVITTST |
| | | RTWALPTYNNHLYKQISNSTSGGSSNDNAYFGYSTPWGYFDFNRFHCHFSPRDWQRLINNN |
| | | WGFRPKRLNFKLFNIQVKEVTDNNGVKTIANNLTSTVQVFTDSDYQLPYVLGSAHEGCLPP |
| | | FPADVFMIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPGPSYRQQR |
| | | VSTTVTQNNNSEFAWPGASSWALNGRNSLMNPGPAMASHKEGEDRFFPLSGSLIFGKQGTG |
| | | RDNVDADKVMITNEEEIKTTNPVATESYGQVATNHQSDGTLAVPFKAQAQTGWVQNQGILP |
| | | GMVWQDRDVYLQGPIWAKIPHTDGNFHPSPLMGGFGMKHPPPQILIKNTPVPADPPTAFNK |
| | | DKLNSFITQYSTGQVSVEIEWELQKENSKRWNPEIQYTSNYYKSNNVEFAVNTEGVYSEPR |
| | | PIGTRYLTRNL |
| VOY201 | 3 | MAADGYLPDWLEDNLSEGIREWWALKPGAPQPKANQQHQDNARGLVLPGYKYLGPGNGLDK |
| | | GEPVNAADAAALEHDKAYDQQLKAGDNPYLKYNHADAEFQERLKEDTSFGGNLGRAVFQAK |
| | | KRLLEPLGLVEEAAKTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWHCDSQWLGDRVITTST |
| | | RTWALPTYNNHLYKQISNSTSGGSSNDNAYFGYSTPWGYFDFNRFHCHFSPRDWQRLINNN |
| | | WGFRPKRLNFKLFNIQVKEVTDNNGVKTIANNLTSTVQVFTDSDYQLPYVLGSAHEGCLPP |
| | | FPADVFMIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPGPSYRQQR |
| | | VSTTVTQNNNSEFAWPGASSWALNGRNSLMNPGPAMASHKEGEDRFFPLSGSLIFGKQGTG |
| | | RDNVDADKVMITNEEEIKTTNPVATESYGQVATNHQSAQTLAVPFKAQAQTGWVQNQGILP |
| | | GMVWQDRDVYLQGPIWAKIPHTDGNFHPSPLMGGFGMKHPPPQILIKNTPVPADPPTAFNK |
| | | DKLNSFITQYSTGQVSVEIEWELQKENSKRWNPEIQYTSNYYKSNNVEFAVNTEGVYSEPR |
| | | PIGTRYLTRNL |
| AAV9/hu. | 11 | MAADGYLPDWLEDNLSEGIREWWALKPGAPQPKANQQHQDNARGLVLPGYKYLGPGNGLDK |
| 14 K449R | | GEPVNAADAAALEHDKAYDQQLKAGDNPYLKYNHADAEFQERLKEDTSFGGNLGRAVFQAK |
| | | KRLLEPLGLVEEAAKTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWHCDSQWLGDRVITTST |
| | | RTWALPTYNNHLYKQISNSTSGGSSNDNAYFGYSTPWGYFDFNRFHCHFSPRDWQRLINNN |
| | | WGFRPKRLNFKLFNIQVKEVTDNNGVKTIANNLTSTVQVFTDSDYQLPYVLGSAHEGCLPP |
| | | FPADVFMIPQYGYLTLNDGSQAVGRSSFYCLEYFPSQMLRTGNNFQFSYEFENVPFHSSYA |
| | | HSQSLDRLMNPLIDQYLYYLSRTINGSGQNQQTLKFSVAGPSNMAVQGRNYIPGPSYRQQR |
| | | VSTTVTQNNNSEFAWPGASSWALNGRNSLMNPGPAMASHKEGEDRFFPLSGSLIFGKQGTG |
| | | RDNVDADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDKLNSFI |
| | | TQYSTGQVSVEIEWELQKENSKRWNPEIQYTSNYYKSNNVEFAVNTEGVYSEPRPIGTRYL |
| | | TRNL |
| AAV9/hu. | 138 | MAADGYLPDWLEDNLSEGIREWWALKPGAPQPKANQQHQDNARGLVLPGYKYLGPGNGLDK |
| 14 WT | | GEPVNAADAAALEHDKAYDQQLKAGDNPYLKYNHADAEFQERLKEDTSFGGNLGRAVFQAK |
| (amino | | KRLLEPLGLVEEAAKTAPGKKRPVEQSPQEPDSSAGIGKSGAQPAKKRLNFGQTGDTESVP |
| acid) | | DPQPIGEPPAAPSGVGSLTMASGGGAPVADNNEGADGVGSSSGNWHCDSQWLGDRVITTST |
| | | RTWALPTYNNHLYKQISNSTSGGSSNDNAYFGYSTPWGYFDFNRFHCHFSPRDWQRLINNN |
| | | WGFRPKRLNFKLFNIQVKEVTDNNGVKTIANNLTSTVQVFTDSDYQLPYVLGSAHEGCLPP |
| | | FPADVFMIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPGPSYRQQR |
| | | VSTTVTQNNNSEFAWPGASSWALNGRNSLMNPGPAMASHKEGEDRFFPLSGSLIFGKQGTG |
| | | RDNVDADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDKLNSFI |
| | | TQYSTGQVSVEIEWELQKENSKRWNPEIQYTSNYYKSNNVEFAVNTEGVYSEPRPIGTRYL |
| | | TRNL |
| AAV9/hu. | 137 | ATGGCTGCCGATGGTTATCTTCCAGATTGGCTCGAGGACAACCTTAGTGAAGGAATTCGCG |
| 14 WT | | AGTGGTGGGCTTTGAAACCTGGAGCCCCTCAACCCAAGGCAAATCAACAACATCAAGACAA |
| (DNA) | | CGCTCGAGGTCTTGTGCTTCCGGGTTACAAATACCTTGGACCCGGCAACGGACTCGACAAG |
| | | GGGGAGCCGGTCAACGCAGCAGACGCGGCGGCCCTCGAGCACGACAAGGCCTACGACCAGC |
| | | AGCTCAAGGCCGGAGACAACCCGTACCTCAAGTACAACCACGCCGACGCCGAGTTCCAGGA |
| | | GCGGCTCAAAGAAGATACGTCTTTTGGGGGCAACCTCGGGCGAGCAGTCTTCCAGGCCAAA |
| | | AAGAGGCTTCTTGAACCTCTTGGTCTGGTTGAGGAAGCGGCTAAGACGGCTCCTGGAAAGA |
| | | AGAGGCCTGTAGAGCAGTCTCCTCAGGAACCGGACTCCTCCGCGGGTATTGGCAAATCGGG |
| | | TGCACAGCCCGCTAAAAAGAGACTCAATTTCGGTCAGACTGGCGACACAGAGTCAGTCCCA |
| | | GACCCTCAACCAATCGGAGAACCTCCCGCAGCCCCCTCAGGTGTGGGATCTCTTACAATGG |
| | | CTTCAGGTGGTGGCGCACCAGTGGCAGACAATAACGAAGGTGCCGATGGAGTGGGTAGTTC |
| | | CTCGGGAAATTGGCATTGCGATTCCCAATGGCTGGGGGACAGAGTCATCACCACCAGCACC |
| | | CGAACCTGGGCCCTGCCCACCTACAACAATCACCTCTACAAGCAAATCTCCAACAGCACAT |
| | | CTGGAGGATCTTCAAATGACAACGCCTACTTCGGCTACAGCACCCCCTGGGGGTATTTTGA |
| | | CTTCAACAGATTCCACTGCCACTTCTCACCACGTGACTGGCAGCGACTCATCAACAACAAC |
| | | TGGGGATTCCGGCCTAAGCGACTCAACTTCAAGCTCTTCAACATTCAGGTCAAAGAGGTTA |
| | | CGGACAACAATGGAGTCAAGACCATCGCCAATAACCTTACCAGCACGGTCCAGGTCTTCAC |
| | | GGACTCAGACTATCAGCTCCCGTACGTGCTCGGGTCGGCTCACGAGGGCTGCCTCCCGCCG |
| | | TTCCCAGCGGACGTTTTCATGATTCCTCAGTACGGGTATCTGACGCTTAATGATGGAAGCC |
| | | AGGCCGTGGGTCGTTCGTCCTTTTACTGCCTGGAATATTTCCCGTCGCAAATGCTAAGAAC |
| | | GGGTAACAACTTCCAGTTCAGCTACGAGTTTGAGAACGTACCTTTCCATAGCAGCTACGCT |
| | | CACAGCCAAAGCCTGGACCGACTAATGAATCCACTCATCGACCAATACTTGTACTATCTCT |
| | | CAAAGACTATTAACGGTTCTGGACAGAATCAACAAACGCTAAAATTCAGTGTGGCCGGACC |
| | | CAGCAACATGGCTGTCCAGGGAAGAAACTACATACCTGGACCCAGCTACCGACAACAACGT |
| | | GTCTCAACCACTGTGACTCAAAACAACAACAGCGAATTTGCTTGGCCTGGAGCTTCTTCTT |
| | | GGGCTCTCAATGGACGTAATAGCTTGATGAATCCTGGACCTGCTATGGCCAGCCACAAAGA |
| | | AGGAGAGGACCGTTTCTTTCCTTTGTCTGGATCTTTAATTTTTGGCAAACAAGGAACTGGA |
| | | AGAGACAACGTGGATGCGGACAAAGTCATGATAACCAACGAAGAAGAAATTAAAACTACTA |
| | | ACCCGGTAGCAACGGAGTCCTATGGACAAGTGGCCACAAACCACCAGAGTGCCCAAGCACA |
| | | GGCGCAGACCGGCTGGGTTCAAAACCAAGGAATACTTCCGGGTATGGTTTGGCAGGACAGA |
| | | GATGTGTACCTGCAAGGACCCATTTGGGCCAAAATTCCTCACACGGACGGCAACTTTCACC |
| | | CTTCTCCGCTGATGGGAGGGTTTGGAATGAAGCACCCGCCTCCTCAGATCCTCATCAAAAA |
| | | CACACCTGTACCTGCGGATCCTCCAACGGCCTTCAACAAGGACAAGCTGAACTCTTTCATC |
| | | ACCCAGTATTCTACTGGCCAAGTCAGCGTGGAGATCGAGTGGGAGCTGCAGAAGGAAAACA |
| | | GCAAGCGCTGGAACCCGGAGATCCAGTACACTTCCAACTATTACAAGTCTAATAATGTTGA |
| | | ATTTGCTGTTAATACTGAAGGTGTATATAGTGAACCCCGCCCCATTGGCACCAGATACCTG |
| | | ACTCGTAATCTGTAA |
| PHP.B | 12 | MAADGYLPDWLEDNLSEGIREWWALKPGAPQPKANQQHQDNARGLVLPGYKYLGPGNGLDK |
| | | GEPVNAADAAALEHDKAYDQQLKAGDNPYLKYNHADAEFQERLKEDTSFGGNLGRAVFQAK |
| | | KRLLEPLGLVEEAAKTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWHCDSQWLGDRVITTST |
| | | RTWALPTYNNHLYKQISNSTSGGSSNDNAYFGYSTPWGYFDFNRFHCHFSPRDWQRLINNN |
| | | WGFRPKRLNFKLFNIQVKEVTDNNGVKTIANNLTSTVQVFTDSDYQLPYVLGSAHEGCLPP |
| | | FPADVFMIPQYGYLTLNDGSQAVGRSSFYCLEYFPSQMLRTGNNFQFSYEFENVPFHSSYA |
| | | HSQSLDRLMNPLIDQYLYYLSRTINGSGQNQQTLKFSVAGPSNMAVQGRNYIPGPSYRQQR |
| | | VSTTVTQNNNSEFAWPGASSWALNGRNSLMNPGPAMASHKEGEDRFFPLSGSLIFGKQGTG |
| | | RDNVDADKVMITNEEEIKTTNPVATESYGQVATNHQSAQTLAVPFKAQAQTGWVQNQGILP |
| | | GMVWQDRDVYLQGPIWAKIPHTDGNFHPSPLMGGFGMKHPPPQILIKNTPVPADPPTAFNK |
| | | DKLNSFITQYSTGQVSVEIEWELQKENSKRWNPEIQYTSNYYKSNNVEFAVNTEGVYSEPR |
| | | PIGTRYLTRNL |
| PHP.N | 13 | MAADGYLPDWLEDNLSEGIREWWALKPGAPQPKANQQHQDNARGLVLPGYKYLGPGNGLDK |
| | | GEPVNAADAAALEHDKAYDQQLKAGDNPYLKYNHADAEFQERLKEDTSFGGNLGRAVFQAK |
| | | KRLLEPLGLVEEAAKTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWHCDSQWLGDRVITTST |
| | | RTWALPTYNNHLYKQISNSTSGGSSNDNAYFGYSTPWGYFDFNRFHCHFSPRDWQRLINNN |
| | | WGFRPKRLNFKLFNIQVKEVTDNNGVKTIANNLTSTVQVFTDSDYQLPYVLGSAHEGCLPP |
| | | FPADVFMIPQYGYLTLNDGSQAVGRSSFYCLEYFPSQMLRTGNNFQFSYEFENVPFHSSYA |
| | | HSQSLDRLMNPLIDQYLYYLSRTINGSGQNQQTLKFSVAGPSNMAVQGRNYIPGPSYRQQR |
| | | VSTTVTQNNNSEFAWPGASSWALNGRNSLMNPGPAMASHKEGEDRFFPLSGSLIFGKQGTG |
| | | RDNVDADKVMITNEEEIKTTNPVATESYGQVATNHQSDGTLAVPFKAQAQTGWVQNQGILP |
| | | GMVWQDRDVYLQGPIWAKIPHTDGNFHPSPLMGGFGMKHPPPQILIKNTPVPADPPTAFNK |
| | | DKLNSFITQYSTGQVSVEIEWELQKENSKRWNPEIQYTSNYYKSNNVEFAVNTEGVYSEPR |
| | | PIGTRYLTRNL |

### Viral Genome of the AAV Particle

In some embodiments, an AAV particle as described herein comprising an AAV capsid polypeptide, e.g., AAV capsid variant, described herein, may be used for the delivery of a viral genome to a tissue (e.g., CNS, DRG, and/or muscle). In some embodiments, an AAV particle comprising an AAV capsid polypeptide, e.g., an AAV capsid variant, described herein can be used for delivery of a viral genome to a tissue or cell, e.g., CNS, DRG, or muscle cell or tissue. In some embodiments, an AAV particle of the present disclosure is a recombinant AAV particle. In some embodiments, an AAV particle of the present disclosure is an isolated AAV particle.

The viral genome may encode any payload, such as but not limited to a polypeptide (e.g., a therapeutic polypeptide), an antibody, an enzyme, an RNAi agent and/or components of a gene editing system. In one embodiment, the AAV particles described herein are used to deliver a payload to cells of the CNS, after intravenous delivery. In another embodiment, the AAV particles described herein are used to deliver a payload to cells of the DRG, after intravenous delivery. In some embodiments, the AAV particles described herein are used to deliver a payload to cells of a muscle, e.g., a heart muscle, after intravenous delivery.

In some embodiments, a viral genome of an AAV particle comprising an AAV capsid polypeptide, e.g., an AAV capsid variant, as described herein, comprises a nucleic acid comprising a transgene encoding a payload In some embodiments, the viral genome comprises an inverted terminal repeat (ITR) sequence. In some embodiments, the viral genome comprises two ITR sequences, e.g., one at the 5′ end of the viral genome (e.g., 5′ relative to the encoded payload) and one at the 3′ end of the viral genome (e.g., 3′ relative to the encoded payload). In some embodiments, a viral genome of the AAV particles described herein (e.g., comprising an AAV capsid variant described herein) may comprise a regulatory element (e.g., promoter), untranslated regions (UTR), a miR binding site a polyadenylation sequence (polyA), a filler or stuffer sequence, an intron, and/or a linker sequence, e.g., for enhancing transgene expression.

In some embodiments, the viral genome components are selected and/or engineered for expression of a payload in a target tissue (e.g., a CNS tissue, a muscle tissue (e.g., heart), or DRG).

### Viral Genome Component: Inverted Terminal Repeats (ITRs)

In some embodiments, the AAV particle comprising an AAV capsid polypeptide, e.g., an AAV capsid variant, described herein comprises a viral genome comprising an ITR and a transgene encoding a payload. In some embodiment, the viral genome has two ITRs. In some embodiments, the two ITRs flank the nucleotide sequence encoding the payload at the 5′ and 3′ ends. In some embodiments, the ITRs function as origins of replication comprising recognition sites for replication. In some embodiments, the ITRs comprise sequence regions which can be complementary and symmetrically arranged. In some embodiments, the ITRs incorporated into viral genomes as described herein may be comprised of naturally occurring polynucleotide sequences or recombinantly derived polynucleotide sequences.

In some embodiments, the ITR may be of the same serotype as the capsid polypeptide, e.g., capsid variant, selected from any of the known serotypes, or a variant thereof. In some embodiments, the ITR may be of a different serotype than the capsid. In one embodiment, the viral genome comprises two ITR sequence regions, wherein the ITRs are of the same serotype as one another. In another embodiment, the viral genome comprises two ITR sequence regions, wherein the ITRs are of different serotypes. Non-limiting examples include zero, one or both of the ITRs having the same serotype as the capsid. In one embodiment both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment, the ITRs are 140-142 nucleotides in length. Non-limiting examples of ITR length are 102, 105, 130, 140, 141, 142, 145 nucleotides in length.

### Viral Genome Component: Promoters

In one embodiment, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid variant described herein comprises a viral genome comprising a nucleic acid comprising a transgene encoding a payload, wherein the transgene is operably linked to a promoter. In some embodiments, the promoter is a species specific promoter, an inducible promoter, tissue-specific, or cell cycle-specific (Parr et al., Nat. Med. 3:1145-9 (1997); the contents of which are herein incorporated by reference in their entirety).

In some embodiments the promoter may be naturally occurring or non-naturally occurring. Non-limiting examples of promoters include those from viruses, plants, mammals, or humans. In some embodiments, the promoters may be those from human cells or systems. In some embodiments, the promoter may be truncated or mutated, e.g., a promoter variant.

In some embodiments, the promoter is a ubiquitous promoter, e.g., capable of expression in multiple tissues. In some embodiments the promoter is an human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter. In some embodiments, the promoter is a cell or tissue specific promoter, e.g., capable of expression in tissues or cells of the central or peripheral nervous systems, regions within (e.g., frontal cortex), and/or sub-sets of cells therein (e.g., excitatory neurons). In some embodiments, the promoter is a cell-type specific promoter capable of expression a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

In some embodiments, the promoter is a liver promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., αMHC, cTnT, and CMV-MLC2k).

In some embodiments, the promoter is a tissue-specific promoter for payload expression in a cell or tissue of the central nervous system. In some embodiments, the promoter is synapsin (Syn) promoter, glutamate vesicular transporter (VGLUT) promoter, vesicular GABA transporter (VGAT) promoter, parvalbumin (PV) promoter, sodium channel Na, 1.8 promoter, tyrosine hydroxylase (TH) promoter, choline acetyltransferase (ChaT) promoter, methyl-CpG binding protein 2 (MeCP2) promoter, Ca²⁺/calmodulin-dependent protein kinase II (CaMKII) promoter, metabotropic glutamate receptor 2 (mGluR2) promoter, neurofilament light (NFL) or heavy (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter. In some embodiments, the promoter is a cell-type specific promoter capable of expression in an astrocyte, e.g., a glial fibrillary acidic protein (GFAP) promoter and a EAAT2 promoter. In some embodiments, the promoter is a cell-type specific promoter capable of expression in an oligodendrocyte, e.g., a myelin basic protein (MBP) promoter.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In some embodiments, the promoter of the viral genome described herein (e.g., comprised within an AAV particle comprising an AAV capsid variant described herein) comprises an EF-1α promoter or variant thereof, e.g., as provided in Table 12. In some embodiments, the EF-1α promoter comprises the nucleotide sequence of any one of SEQ ID NOs: 19, 20, 22, 23, 27, 28, 30-39 or any one of the sequences provided in Table 12, a nucleotide sequence comprising at least one, two, or three but no more than four modifications, e.g., substitutions, relative to the nucleotide sequence of SEQ ID NOs: 19, 20, 22, 23, 27, 28, 30-39 or any one of the sequences provided in Table 12, or a nucleotide sequence with at least 70% (e.g., 80, 85%, 90%, 95%, 96%, 97%, 98%, or 99%) sequence identity to any one of SEQ ID NOs: 19, 20, 22, 23, 27, 28, 30-39 or any one of the sequences provided in Table 12.

TABLE 12
Exemplary Promoter Variants

| | | SEQ |
|---|---|---|
| | | ID |
| Description | Sequences | NO: |
| EF1a Promoter | CGTGAGGCTCCGGTGCCCGTCAGTGGGCAGAGCGCACATCGCCCACAGTCCCCGAGA | 19 |
| (intron | AGTTGGGGGGAGGGGTCGGCAATTGAACCGGTGCCTAGAGAAGGTGGCGCGGGGTAA | |
| underlined) | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACAGGTAAGTGCCGTGTGTGGTTCCCGCGGGCCTGGCCTCTTTACGGGTTATG | |
| | GCCCTTGCGTGCCTTGAATTACTTCCACCTGGCTGCAGTACGTGATTCTTGATCCCG | |
| | AGCTTCGGGTTGGAAGTGGGTGGGAGAGTTCGAGGCCTTGCGCTTAAGGAGCCCCTT | |
| | CGCCTCGTGCTTGAGTTGAGGCCTGGCCTGGGCGCTGGGGCCGCCGCGTGCGAATCT | |
| | GGTGGCACCTTCGCGCCTGTCTCGCTGCTTTCGATAAGTCTCTAGCCATTTAAAATT | |
| | TTTGATGACCTGCTGCGACGCTTTTTTTCTGGCAAGATAGTCTTGTAAATGCGGGCC | |
| | AAGATCTGCACACTGGTATTTCGGTTTTTGGGGCCGCGGGCGGCGACGGGGCCCGTG | |
| | CGTCCCAGCGCACATGTTCGGCGAGGCGGGGCCTGCGAGCGCGGCCACCGAGAATCG | |
| | GACGGGGGTAGTCTCAAGCTGGCCGGCCTGCTCTGGTGCCTGGCCTCGCGCCGCCGT | |
| | GTATCGCCCCGCCCTGGGCGGCAAGGCTGGCCCGGTCGGCACCAGTTGCGTGAGCGG | |
| | AAAGATGGCCGCTTCCCGGCCCTGCTGCAGGGAGCTCAAAATGGAGGACGCGGCGCT | |
| | CGGGAGAGCGGGCGGGTGAGTCACCCACACAAAGGAAAAGGGCCTTTCCGTCCTCAG | |
| | CCGTCGCTTCATGTGACTCCACGGAGTACCGGGCGCCGTCCAGGCACCTCGATTAGT | |
| | TCTCGAGCTTTTGGAGTACGTCGTCTTTAGGTTGGGGGGAGGGGTTTTATGCGATGG | |
| | AGTTTCCCCACACTGAGTGGGTGGAGACTGAAGTTAGGCCAGCTTGGCACTTGATGT | |
| | AATTCTCCTTGGAATTTGCCCTTTTTGAGTTTGGATCTTGGTTCATTCTCAAGCCTC | |
| | AGACAGTGGTTCAAAGTTTTTTTCTTCCATTTCAGGTGTCGTGA | |
| miniEF1a | GCCCGTCAGTGGGCAGAGCGCACATCGCCCACAGTCCCCGAGAAGTTGGGGGGAGGG | 20 |
| | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACGCGTAAG | |
| Promoter | GCATG | |
| Variant 1 | | |
| Promoter | GGTGGAGAAGAGCATG | 22 |
| Variant 2 | | |
| Promoter | GTCATCACTGAGGTGGAGAAGAGCATG | 23 |
| Variant 3 | | |
| Promoter | CGTGAG | |
| Variant 4 | | |
| Promoter | GT | |
| Variant 5 | | |
| Promoter | GCTCCGGT | |
| Variant 6 | | |
| Promoter | GCCCGTCAGTGGGCAGAGCGCACATCGCCCACAGTCCCCGAGAAGTTGGGGGGAGGG | 27 |
| Variant | GTCGGCAATTGAACCGGTGCCTAGAGAAGGTGGCGCGGGGTAAACTGGGAAAGTGAT | |
| 19 | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACAG | |
| Promoter | GCCCGTCAGTGGGCAGAGCGCACATCGCCCACAGTCCCCGAGAAGTTGGGGGGAGGG | 28 |
| Variant | GTCGGCAATTGAACCGGTGCCTAGAGAAGGTGGCGCGGGGTAAACTGGGAAAGTGAT | |
| 20 | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACGC | |
| Promoter | GTAAG | |
| Variant 7 | | |
| Promoter | GTGCCCGTCAGTGGGCAGAGCGCACATCGCCCACAGTCCCCGAGAAGTTGGGGGGAG | 30 |
| Variant | GGGTCGGCAATTGAACCGGTGCCTAGAGAAGGTGGCGCGGGGTAAACTGGGAAAGTG | |
| 8 | ATGTCGTGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACACGCGTAA | |
| | G | |
| Promoter | GCTCCGGTGCCCGTCAGTGGGCAGAGCGCACATCGCCCACAGTCCCCGAGAAGTTGG | 31 |
| Variant | GGGGAGGGGTCGGCAATTGAACCGGTGCCTAGAGAAGGTGGCGCGGGGTAAACTGGG | |
| 9 | AAAGTGATGTCGTGTACTGGCTCCGCCTTTTTCCCGAGGGTGGGGGAGAACCGTATA | |
| | TAAGTGCAGTAGTCGCCGTGAACGTTCTTTTTCGCAACGGGTTTGCCGCCAGAACAC | |
| | GCGTAAG | |
| Promoter | CGTGAGGCTCCGGTGCCCGTCAGTGGGCAGAGCGCACATCGCCCACAGTCCCCGAGA | 32 |
| Variant | AGTTGGGGGGAGGGGTCGGCAATTGAACCGGTGCCTAGAGAAGGTGGCGCGGGGTAA | |
| 10 | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACGCGTAAG | |
| Promoter | CGTGAGGCTCCGGTGCCCGTCAGTGGGCAGAGCGCACATCGCCCACAGTCCCCGAGA | 33 |
| Variant | AGTTGGGGGGAGGGGTCGGCAATTGAACCGGTGCCTAGAGAAGGTGGCGCGGGGTAA | |
| 11 | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACAG | |
| Promoter | GCATGCGTGAGGCTCCGGTGCCCGTCAGTGGGCAGAGCGCACATCGCCCACAGTCCC | 34 |
| Variant | CGAGAAGTTGGGGGGAGGGGTCGGCAATTGAACCGGTGCCTAGAGAAGGTGGCGCGG | |
| 12 | GGTAAACTGGGAAAGTGATGTCGTGTACTGGCTCCGCCTTTTTCCCGAGGGTGGGGG | |
| | AGAACCGTATATAAGTGCAGTAGTCGCCGTGAACGTTCTTTTTCGCAACGGGTTTGC | |
| | CGCCAGAACACGCGTAAG | |
| Promoter | GCATGCGTGAGGCTCCGGTGCCCGTCAGTGGGCAGAGCGCACATCGCCCACAGTCCC | 35 |
| Variant | CGAGAAGTTGGGGGGAGGGGTCGGCAATTGAACCGGTGCCTAGAGAAGGTGGCGCGG | |
| 13 | GGTAAACTGGGAAAGTGATGTCGTGTACTGGCTCCGCCTTTTTCCCGAGGGTGGGGG | |
| | AGAACCGTATATAAGTGCAGTAGTCGCCGTGAACGTTCTTTTTCGCAACGGGTTTGC | |
| | CGCCAGAACACAG | |
| Promoter | GGTGGAGAAGAGCATGCGTGAGGCTCCGGTGCCCGTCAGTGGGCAGAGCGCACATCG | 36 |
| Variant | CCCACAGTCCCCGAGAAGTTGGGGGGAGGGGTCGGCAATTGAACCGGTGCCTAGAGA | |
| 14 | AGGTGGCGCGGGGTAAACTGGGAAAGTGATGTCGTGTACTGGCTCCGCCTTTTTCCC | |
| | GAGGGTGGGGGAGAACCGTATATAAGTGCAGTAGTCGCCGTGAACGTTCTTTTTCGC | |
| | AACGGGTTTGCCGCCAGAACACGCGTAAG | |
| Promoter | GGTGGAGAAGAGCATGCGTGAGGCTCCGGTGCCCGTCAGTGGGCAGAGCGCACATCG | 37 |
| Variant | CCCACAGTCCCCGAGAAGTTGGGGGGAGGGGTCGGCAATTGAACCGGTGCCTAGAGA | |
| 15 | AGGTGGCGCGGGGTAAACTGGGAAAGTGATGTCGTGTACTGGCTCCGCCTTTTTCCC | |
| | GAGGGTGGGGGAGAACCGTATATAAGTGCAGTAGTCGCCGTGAACGTTCTTTTTCGC | |
| | AACGGGTTTGCCGCCAGAACACAG | |
| Promoter | GTCATCACTGAGGTGGAGAAGAGCATGCGTGAGGCTCCGGTGCCCGTCAGTGGGCAG | 38 |
| Variant | AGCGCACATCGCCCACAGTCCCCGAGAAGTTGGGGGGAGGGGTCGGCAATTGAACCG | |
| 16 | GTGCCTAGAGAAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCGCCGTGAACG | |
| | TTCTTTTTCGCAACGGGTTTGCCGCCAGAACACGCGTAAG | |
| Promoter | GTCATCACTGAGGTGGAGAAGAGCATGCGTGAGGCTCCGGTGCCCGTCAGTGGGCAG | 39 |
| Variant 18 | AGCGCACATCGCCCACAGTCCCCGAGAAGTTGGGGGGAGGGGTCGGCAATTGAACCG | |
| | GTGCCTAGAGAAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCGCCGTGAACG | |
| | TTCTTTTTCGCAACGGGTTTGCCGCCAGAACACAG | |

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo(dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In one embodiment, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprises at least one artificial UTR which is not a variant of a wild type UTR.

In one embodiment, the viral genome of the AAV particle comprises UTRs which have been selected from a family of transcripts whose proteins share a common function, structure, feature or property.

### Viral Genome Component: Polyadenylation Sequence

The viral genome of the AAV particle described herein (e.g., an AAV particle comprising an AAV capsid polypeptide, e.g., an AAV capsid variant, described herein) may comprise a polyadenylation sequence. In some embodiments, the viral genome of the AAV particle (e.g., an AAV particle comprising an AAV capsid polypeptide, e.g., an AAV capsid variant, described herein) comprises a polyadenylation sequence between the 3′ end of the nucleotide sequence encoding the payload and the 5′ end of the 3′ITR.

### Viral Genome Component: Introns

In some embodiments, the viral genome of the AAV particle as described herein (e.g., an AAV particle comprising an AAV capsid polypeptide, e.g., an AAV capsid variant), comprises an element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an intron. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

### Viral Genome Component: Stuffer Sequences

In some embodiments, the viral genome of an AAV particle described herein (e.g., an AAV particle comprising an AAV capsid polypeptide, e.g., an AAV capsid variant), comprises an element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

Viral Genome Component: miRNA

In some embodiments, the viral genome comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and B-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles disclosed herein can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome disclosed herein, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleotide sequence encoding a payload), e.g., before the polyA sequence, 5′-UTR region of the viral genome (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complimentary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complimentary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183, or a miR1).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 3672), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3672, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCACACT CCA (SEQ ID NO: 3673), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3673, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4679), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 4679, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 3674), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 3674, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 3675), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3675, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g., an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 3676), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3676, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In certain embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 3677), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3677, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

### Genome Size

In one embodiment, the AAV particle described herein (e.g., an AAV particle comprising an AAV capsid polypeptide, e.g., an AAV capsid variant), may comprise a single-stranded or double-stranded viral genome. The size of the viral genome may be small, medium, large or the maximum size. As described above, the viral genome may comprise a promoter and a polyA tail.

In one embodiment, the viral genome may be a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In one embodiment, the viral genome may be a small double stranded viral genome. A small double stranded viral genome may be 1.3 to 1.7 kb in size such as, but not limited to, about 1.3, 1.4, 1.5, 1.6, and 1.7 kb in size.

In one embodiment, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In one embodiment, the viral genome may be a medium double stranded viral genome. A medium double stranded viral genome may be 1.8 to 2.1 kb in size such as, but not limited to, about 1.8, 1.9, 2.0, and 2.1 kb in size.

In one embodiment, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In one embodiment, the viral genome may be a large double stranded viral genome. A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Payloads

In some embodiments, an AAV particle of the present disclosure (e.g. an AAV particle comprising an AAV capsid polypeptide, e.g., an AAV capsid variant) comprises a viral genome comprising a nucleic acid encoding a payload. In some embodiments, the encoded payload is an RNAi agent or a polypeptide. A payload of the present disclosure may be, but is not limited to, a peptide, a polypeptide, a protein, an antibody, an RNAi agent, etc.

In some embodiments, the nucleotide sequence encoding a payload may comprise a combination of coding and non-coding nucleic acid sequences. In some embodiments, the nucleotide sequence encoding the payload may encode a coding or non-coding RNA.

In some embodiments, the AAV particles described herein, e.g., an AAV particle comprising an AAV capsid polypeptide, e.g., an AAV capsid variant, comprises a nucleic acid encoding a payload. In some embodiments, the encoded payload comprises a therapeutic protein, an antibody, an enzyme, one or more components of a genome editing system, and/or an RNAi agent (e.g., a dsRNA, siRNA, shRNA, pre-miRNA, pri-miRNA, miRNA, stRNA, lncRNA, piRNA, or snoRNA). In some embodiments, the encoded payload modulates, e.g., increases or decreases, the presence, level, and/or activity of a gene, mRNA, protein, or a combination thereof, e.g., in a cell or a tissue.

### Polypeptides

In some embodiments, the encoded payload of AAV particle comprising an AAV capsid polypeptide, e.g., an AAV capsid variant, described herein comprises a polypeptide, protein, or peptide, e.g., a polypeptide, protein, or peptide described herein. The nucleic acid encoding the payload, may encode a product of any known gene and/or a recombinant version thereof. In some embodiments, the nucleic acid encoding the payload may encode at least one allele of apolipoprotein E (APOE) such as, but not limited to ApoE2, ApoE3 and/or ApoE4. In one embodiment, the nucleic acid encoding the payload encodes ApoE2 (cys112, cys158) protein or a fragment or variant thereof. In one embodiment, the nucleic acid encoding the payload encodes an ApoE3 (cys112, arg158) protein or fragment or variant thereof. In one embodiment, the nucleic acid encoding the payload encodes ApoE4 (arg112, arg158). As another non-limiting example, the encoded payload comprises an aromatic L-amin acid decarboxylase (AADC) protein. As another non-limiting example, the encoded payload comprises an antibody, or a fragment thereof. As another non-limiting example, the encoded payload comprises a human survival of motor neuron (SMN) 1 or SMN2 protein, or fragments or variants thereof. As another non-limiting example, the encoded payload region comprises a glucocerebrosidase (GBA1) protein, or a fragment or variant thereof. As another non-limiting example, the encoded payload comprises a granulin precursor or progranulin (GRN) protein, or a fragment or variant thereof. As another non-limiting example, the encoded payload comprises an aspartoacylase (ASPA) protein, or a fragment or variant thereof. As another non-limiting example, the encoded payload comprises a tripeptidyl peptidase I (CLN2) protein, or a fragment or variant thereof. As another non-limiting example, the encoded payload comprises a beta-galactosidase (GLB1) protein, or a fragment or variant thereof. As another non-limiting example, the encoded payload comprises a N-sulphoglucosamine sulphohydrolase (SGSH) protein, or a fragment or variant thereof. As another non-limiting example, the encoded payload comprises an N-acetyl-alpha-glucosaminidase (NAGLU) protein, or a fragment or variant thereof. As another non-limiting example, the encoded payload comprises an iduronate 2-sulfatase (IDS) protein, or a fragment or variant thereof. As another non-limiting example, the encoded payload comprises an intracellular cholesterol transporter (NPC1) protein, or a fragment or variant thereof. As another non-limiting example, the encoded payload comprises a gigaxonin (GAN) protein, or a fragment or variant thereof. The AAV viral genomes encoding polypeptides described herein may be useful in the fields of human disease, viruses, infections veterinary applications and a variety of in vivo and in vitro settings.

Amino acid sequences of a payload polypeptide encoded by a viral genome described herein, may be translated as a whole polypeptide, a plurality of polypeptides or fragments of polypeptides, which independently may be encoded by one or more nucleic acids, fragments of nucleic acids or variants of any of the aforementioned.

### Antibodies and Antibody Binding Fragments

In some embodiments, the encoded payload of AAV particle comprising an AAV capsid variant described herein comprises an antibody or antibody binding fragment. In some embodiments, the antibody may be a full antibody, a fragment, or any functional variant thereof. As non-limiting examples, an antibody may be a native antibody (e.g., with two heavy and two light chains), a heavy chain variable region, a light chain variable region, a heavy chain constant region, a light chain constant region, Fab, Fab′, F(ab′)₂, Fv, or scFv fragments, a diabody, a linear antibody, a single-chain antibody, a multi-specific antibody, an intrabody, one or more heavy chain complementarity determining regions (CDR), one or more light chain CDRs, a bi-specific antibody, a monoclonal antibody, a polyclonal antibody, a humanized antibody, an antibody mimetic, an antibody variant, a miniaturized antibody, a unibody, a maxibody, and/or a chimeric antigen receptor. The encoded antibody or antibody binding fragment may be useful in the treatment of a neurological disease, a neurodegenerative disorder, a muscular disease, a neuromuscular disorder, a neuro-oncological disorder, or any disorder associated with the central and/or peripheral nervous systems.

In some embodiments, the viral genome of the AAV particle (e.g., an AAV particle comprising an AAV capsid variant described herein) may comprise a nucleic acid which has been engineered to enable or enhance the expression of an antibody, or antibody binding fragment thereof.

In some embodiments, the encoded antibody of the payload of an AAV particle comprising an AAV capsid variant, described herein comprises at least one immunoglobulin variable domain sequence. An antibody may include, for example, full-length, mature antibodies and antigen-binding fragments of an antibody. For example, an antibody can include a heavy (H) chain variable domain sequence (VH), and a light (L) chain variable domain sequence (VL). In another example, an antibody includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments, e.g., an antibody binding fragments, retain the ability to selectively bind with their respective antigen or receptor.

In some embodiments, the antibody binding fragment comprises at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antigen binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005).

In some embodiments, the encoded antibody of the payload of an AAV particle described herein comprises a multispecific antibody, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody comprises a third, fourth or fifth immunoglobulin variable domain. In some embodiments, a multispecific antibody is a bispecific antibody, a trispecific antibody, or tetraspecific antibody.

In some embodiments, an encoded multispecific antibody of the payload of an AAV particle described herein is an encoded bispecific antibody. A bispecific antibody has specificity for no more than two antigens. A bispecific antibody is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein).

An antibody or an antibody binding fragment encoded by a viral genome of an AAV particle described herein, may be, but is not limited to, an antibody or antibody fragment that binds to β-amyloid, APOE, tau, SOD1, TDP-43, huntingtin, and/or synuclein. In some embodiments, the encoded payload comprises an antibody or antibody fragment that binds to a neuro-oncology related target, e.g., HER2, EGFR (e.g., EGFRvIII). In some embodiments, the encoded payload comprises an antibody that binds to HER2/neu. In some embodiments, the encoded payload comprises an antibody that binds to β-amyloid. In some embodiments, the encoded payload comprises an antibody that binds to tau.

### Gene Editing System

In some embodiments, the encoded payload of AAV particle comprising an AAV capsid variant described herein comprises a gene editing system or one or more components thereof. In some embodiments, the gene editing system comprises nucleic acid sequences that encode proteins having enzymatic activity to (i) selectively induce double or single stranded breaks in a DNA or RNA sequence, or (ii) substitute, insert or delete a particular base or set of bases of a DNA or RNA sequence in the absence of a double or single stranded break in the DNA or RNA. In some embodiments, the gene editing system includes, but is not limited to a CRISPR-Cas system (including different Cas or Cas-related nucleases), a Zinc finger nuclease, a meganuclease, a TALEN or a base editors. In some embodiments, the gene editing system comprises a chromosomal integration of a transgene, e.g., introduced by a parvovirus vector in the absence of an exogenous nuclease or an enzymatic entity.

### RNAi Agents

In some embodiments, the encoded payload of AAV particle comprising an AAV capsid variant described herein comprises an RNAi agent, e.g., an RNAi agent described herein. In some embodiments, the encoded payload of a viral genome of an AAV particle comprising an AAV capsid variant described herein comprises an RNAi agent, the RNAi, such as but not limited to, a dsRNA, a siRNA, a shRNA, a pre-miRNA, a pri-miRNA, a miRNA, a stRNA, a lncRNA, a piRNA, or a snoRNA. In some embodiments, the encoded payload comprises an RNAi agent for inhibiting expression of a SOD1, MAPT, APOE, HTT, C9ORF72, TDP-43, APP, BACE, SNCA, ATXN1, ATXN3, ATXN7, SCN1A-SCN5A, or SCN8A-SCN11A gene, protein, and/or mRNA. In some embodiments, the RNAi agent encoded by a viral genome described herein inhibits SOD1, MAPT, APOE, HTT, C9ORF72, TDP-43, APP, BACE, SNCA, ATXN1, ATXN3, ATXN7, SCN1A-SCN5A, or SCN8A-SCN11A.

An AAV particle comprising an AAV capsid variant described herein may comprise a viral genome encoding an RNAi agent, which targets the mRNA of a gene to modulate, e.g., interfere with gene expression and/or protein production.

In some embodiments, the RNAi agent may target a gene at the location of a single-nucleotide polymorphism (SNP) or variant within the nucleotide sequence of the gene.

The RNAi agent may be an siRNA duplex, wherein the siRNA duplex contains an antisense strand (guide strand) and a sense strand (passenger strand) hybridized together forming a duplex structure, wherein the antisense strand is complementary to the nucleic acid sequence of the targeted gene, and wherein the sense strand is homologous to the nucleic acid sequence of the targeted gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

Each strand of an siRNA duplex targeting a gene of interest may be about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length.

In one embodiment, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding the target gene, and the region of complementarity is 30 nucleotides or less, and at least 15 nucleotides in length. Generally, the dsRNA is 19 to 25, 19 to 24 or 19 to 21 nucleotides in length. In some embodiments, the dsRNA is from about 15 to about 25 nucleotides in length, and in other embodiments the dsRNA is from about 25 to about 30 nucleotides in length. In some embodiments, the dsRNA is about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

In some embodiments, the encoded RNAi agent is an siRNA.

In some embodiments, the RNAi agent, e.g., an RNAi agent described herein inhibits the expression of the gene, mRNA, and/or protein by at least 10%, at least 20%, at least 25%, at least 30%, at least 35% or at least 40% or more, such as when assayed by a method known in the art. In some embodiments, the RNAi agent inhibits expression of a gene, mRNA, and protein by 50-100%, e.g., by 30%, 40%, 50%, 60%, 70%, 80%, 85%, 90%, 95% and 100%.

In some embodiments, the AAV particle described herein, comprising a viral genome encoding an RNAi agent targeting a gene of interest is administered to a subject in need for treating and/or ameliorating a disease, e.g., a neurological disorder of any disease associated with the central or peripheral nervous systems.

Design of siRNA

An AAV particle described herein (e.g., an AAV particle comprising an AAV capsid variant described herein) may comprise a viral genome encoding a siRNA molecule (e.g., siRNA duplex or encoded dsRNA) that target a gene of interest and suppress target gene expression, mRNA expression, and protein production. In some aspects, the siRNA molecules are designed and used to knock out target gene variants in cells, e.g., transcripts that are identified in neurological disease. In some aspects, the siRNA molecules are designed and used to knock down target gene variants in cells.

Some guidelines for designing siRNAs (for insertion into a viral genome of the AAV particles described herein) have been proposed in the art. These guidelines generally recommend generating a 19-nucleotide duplexed region, symmetric 2-3 nucleotide 3′ overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such considerations, together with the specific sequence of a target gene, highly effective siRNA molecules essential for suppressing mammalian target gene expression may be readily designed.

In one embodiment, the sense and/or antisense strand is designed based on the method and rules outlined in European Patent Publication No. EP1752536, the contents of which are herein incorporated by reference in their entirety. As a non-limiting example, the 3′-terminal base of the sequence is adenine, thymine or uracil. As a non-limiting example, the 5′-terminal base of the sequence is guanine or cytosine. As a non-limiting example, the 3′-terminal sequence comprises seven bases rich in one or more bases of adenine, thymine and uracil.

In one embodiment, an siRNA molecule comprises a sense strand and a complementary antisense strand in which both strands are hybridized together to form a duplex structure. The antisense strand has sufficient complementarity to the target mRNA sequence to direct target-specific RNAi, e.g., the siRNA molecule has a sequence sufficient to trigger the destruction of the target mRNA by the RNAi machinery or process.

In some embodiments, the antisense strand and target mRNA sequences have 100% complementarity. The antisense strand may be complementary to any part of the target mRNA sequence. Neither the identity of the sense sequence nor the homology of the antisense sequence need be 100% complementary to the target.

In other embodiments, the antisense strand and target mRNA sequences comprise at least one mismatch. As a non-limiting example, the antisense strand and the target mRNA sequence have at least 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The siRNA molecule may have a length from about 10-50 or more nucleotides, e.g., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In one embodiment, the siRNA molecule has a length from about 19 to 25, 19 to 24 or 19 to 21 nucleotides.

In some embodiments, the siRNA molecule can be a synthetic RNA duplex comprising about 19 nucleotides to about 25 nucleotides, and two overhanging nucleotides at the 3′-end.

The siRNA molecule may comprise an antisense sequence and a sense sequence, or a fragment or variant thereof. As a non-limiting example, the antisense sequence and the sense sequence have at least 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The sense and antisense sequences may be completely complementary across a substantial portion of their length. In other embodiments, the sense sequence and antisense sequence may be at least 70, 80, 90, 95 or 99% complementary across independently at least 50, 60, 70, 80, 85, 90, 95, or 99% of the length of the strands.

In some embodiments, the sense and antisense strands of a siRNA duplex are linked by a short spacer sequence leading to the expression of a stem-loop structure termed short hairpin RNA (shRNA). The hairpin is recognized and cleaved by Dicer, thus generating mature siRNA molecules.

In some embodiments, the siRNA molecules, as well as associated spacer and/or flanking regions once designed, can be encoded by the viral genome of the AAV particles described herein, for delivery to a cell.

### Molecular Scaffold

In some embodiments, the siRNA molecules may be encoded in a modulatory polynucleotide which also comprises a molecular scaffold.

In some embodiments, the modulatory polynucleotide which comprises the payload (e.g., siRNA, miRNA or other RNAi agent described herein) includes a molecular scaffold which comprises a 5′ flanking sequence, a loop region, and/or a 3′ flanking region. In some embodiments a 5′ or 3′ flanking region may be of any length and may a wild type microRNA sequence or a portion thereof, or may be completely artificial. A 3′ flanking sequence may mirror the 5′ flanking sequence in size and origin. Either flanking sequence may be absent. In one embodiment, both the 5′ and 3′ flanking sequences are absent. The 3′ flanking sequence may optionally contain one or more CNNC motifs, where “N” represents any nucleotide. In some embodiments, the loop comprises at least one UGUG motif. In some embodiments, the UGUG motif is located at the 5′ terminus of the loop. In some embodiments the 5′ and 3′ flanking sequences are the same sequence. In some embodiments they differ by 2%, 3%, 4%, 5%, 10%, 20% or more than 30% when aligned to each other.

In some embodiments, modulatory polynucleotide comprises a stem loop structure. In some embodiments, the modulatory polynucleotide comprises in 5′ to 3′ order: a 5′ flanking sequence, a guide strand sequence, a loop region, a passenger strand sequence, and a 3′ flanking sequence. In some embodiments, the modulatory polynucleotide comprises in 5′ to 3′ order: a 5′ flanking sequence, a passenger strand sequence, a loop region, a guide strand sequence, and a 3′ flanking sequence.

In one embodiment, the molecular scaffold comprises a dual-function targeting modulatory polynucleotide.

In one embodiment, the molecular scaffold may comprise one or more linkers known in the art. The linkers may separate regions or one molecular scaffold from another. As a non-limiting example, the molecular scaffold may be polycistronic.

In one embodiment, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

### AAV Production

Viral production disclosed herein describes processes and methods for producing AAV particles (with enhanced, improved and/or increased tropism for a target tissue), e.g., an AAV particle comprising an AAV capsid variant that may be used to contact a target cell to deliver a payload.

In some embodiments, disclosed herein is a method of making AAV particle of the present disclosure, e.g., an AAV particle comprising an AAV capsid variant the method comprising: (i) providing a host cell comprising a viral genome described herein and (ii) incubating the host cell under conditions suitable to enclose the viral genome in an AAV capsid variant, e.g., an AAV capsid variant described herein (e.g., an AAV capsid variant listed in Tables 3, 4, or 5), thereby making the AAV particle. In some embodiments, the method comprises prior to step (i), introducing a first nucleic acid comprising the viral genome into a cell. In some embodiments, the host cell comprises a second nucleic acid encoding the AAV capsid variant. In some embodiments, the second nucleic acid is introduced into the host cell prior to, concurrently with, or after the first nucleic acid molecule. In some embodiments, the AAV particle described herein is an isolated AAV particle. In some embodiments, the AAV particle described herein is a recombinant AAV particle.

Any method known in the art may be used for the preparation of AAV particles. In some embodiments, AAV particles are produced in mammalian cells (e.g., HEK293). In another embodiment, AAV particles are produced in insect cells (e.g., Sf9).

Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in International Patent Publication WO2015191508, the contents of which are herein incorporated by reference in their entirety.

### Therapeutic Applications

The present disclosure provides a method for treating a disease, disorder and/or condition in a subject, including a human subject, comprising administering to the subject an AAV particle described herein, e.g., an AAV particle comprising an AAV capsid variant (e.g., an AAV capsid variant described herein), or administering to the subject any of the described compositions, including a pharmaceutical composition, described herein.

In some embodiments, the AAV particle of the present disclosure (e.g., an AAV particle comprising an AAV capsid variant) are administered to a subject prophylactically, to prevent on-set of disease. In another embodiment, the AAV particle (e.g., an AAV particle comprising an AAV capsid variant) of the present disclosure are administered to treat (e.g., lessen the effects of) a disease or symptoms thereof. In yet another embodiment, the AAV particle of the present disclosure (e.g., an AAV particle comprising an AAV capsid variant) are administered to cure (eliminate) a disease. In another embodiment, the AAV particle (e.g., an AAV particle comprising an AAV capsid variant) of the present disclosure are administered to prevent or slow progression of disease. In yet another embodiment, the AAV particle (e.g., an AAV particle comprising an AAV capsid variant) of the present disclosure are used to reverse the deleterious effects of a disease. Disease status and/or progression may be determined or monitored by standard methods known in the art.

In some embodiments, the AAV particle of the disclosure (e.g., an AAV particle comprising an AAV capsid variant) is useful for treatment, prophylaxis, palliation or amelioration of a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein (e.g., an AAV particle comprising an AAV capsid variant described herein) for a gene replacement therapy.

In some embodiments, provided herein is method for treating a neurological disorder and/or neurodegenerative disorder in a subject, comprising administering to the subject an effective amount of a pharmaceutical composition described herein or an AAV particle, e.g., a plurality of particles, comprising an AAV capsid variant described herein. In some embodiments, treatment of a neurological disorder and/or neurodegenerative disorder comprises prevention of said neurological disorder and/or neurological disorder.

In some embodiments, the AAV particle (e.g., an AAV particle comprising an AAV capsid variant) of the disclosure is useful for the treatment, prophylaxis, palliation or amelioration of neurological diseases and/or disorders. In some embodiments, the AAV particle of the disclosure (e.g., an AAV particle comprising an AAV capsid variant) is useful for the treatment, prophylaxis, palliation or amelioration of tauopathy.

In some embodiments, the AAV particle of the disclosure (e.g., an AAV particle comprising an AAV capsid variant) is for the treatment, prophylaxis, palliation or amelioration of Alzheimer's Disease. In some embodiments, treatment of Alzheimer's Disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ApoE2 protein, ApoE4 protein, an ApoE3 protein, BDNF protein, CYP46A1 protein, Klotho protein, fractalkine (FKN) protein, neprilysin protein (NEP), CD74 protein, caveolin-1, or a combination or variant thereof. In some embodiments, treatment of Alzheimer's Disease comprises the use of an AAV particle described herein (e.g., an AAV particle comprising an AAV capsid variant described herein) for a reduction in the expression of a tau gene and/or protein, a synuclein gene and/or protein, or a combination or variant thereof. In some embodiments, the payload encoded by an AAV particle comprising a capsid variant described herein comprises an antibody that binds to tau or synuclein, an RNAi agent for inhibiting tau or synuclein, a gene editing system (e.g., a CRISPR-Cas system) for altering tau or synuclein expression, or a combination thereof.

In some embodiments, the AAV particle of the disclosure (e.g., an AAV particle comprising an AAV capsid variant) is for the treatment, prophylaxis, palliation or amelioration of frontal temporal dementia. In some embodiments, treatment of frontal temporal dementia comprises the use of an AAV particle described herein (e.g., an AAV particle comprising an AAV capsid variant described herein) for a gene replacement therapy. In some embodiments, the payload encoded by an AAV particle comprising a capsid variant described herein comprises a progranulin protein or variant thereof.

In some embodiments, the AAV particle of the disclosure (e.g., an AAV particle comprising an AAV capsid variant) is useful the treatment, prophylaxis, palliation or amelioration of Friedreich's ataxia, or any disease stemming from a loss or partial loss of frataxin protein. In some embodiments, treatment of Friedreich's ataxia comprises the use of an AAV particle described herein (e.g., an AAV particle comprising an AAV capsid variant described herein) for a gene replacement therapy.

In some embodiments, the AAV particle of the disclosure (e.g., an AAV particle comprising an AAV capsid variant) is useful for the treatment, prophylaxis, palliation or amelioration of Parkinson's Disease. In some embodiments, treatment of Parkinson's disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an AADC protein, GAD protein, GDNF protein, TH-GCH1 protein, GBA protein, AIMP2-DX2 protein, or a combination or variant thereof. In some embodiments, treatment of Parkinson's disease comprises the use of an AAV particle described herein (e.g., an AAV particle comprising an AAV capsid variant described herein) for a gene knock-down therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an alpha-synuclein gene, mRNA, and/or protein, or variant thereof.

In some embodiments, the AAV particle of the disclosure (e.g., an AAV particle comprising an AAV capsid variant) is useful for the treatment, prophylaxis, palliation or amelioration of an AADC deficiency. In some embodiments, treatment of AADC deficiency comprises the use of an AAV particle described herein (e.g., an AAV particle comprising an AAV capsid variant described herein) for a gene replacement therapy. In some embodiments, the payload encoded by an AAV particle comprising a capsid variant described herein comprises an AADC protein or variant thereof.

In some embodiments, the AAV particle of the disclosure (e.g., an AAV particle comprising an AAV capsid variant) is useful for the treatment, prophylaxis, palliation or amelioration of Amyotrophic lateral sclerosis (ALS). In some embodiments, treatment of ALS comprises the use of an AAV particle described herein (e.g., an AAV particle comprising an AAV capsid variant described herein) for a gene replacement therapy. In some embodiments, the payload encoded by an AAV particle comprising a capsid variant described herein comprises an TDP-43 protein, UPF1 protein, C9orf72 protein, CCNF protein, HSF1 protein, Factor H protein, NGF protein, ADAR2 protein, GDNF protein, VEGF protein, HGF protein, NRTN protein, AIMP2-DX2 protein, or a combination or variant thereof. In some embodiments, treatment of ALS comprises the use of an AAV particle described herein (e.g., an AAV particle comprising an AAV capsid variant described herein) for a gene knock-down therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of a SOD1 or C9ORF72 gene, mRNA, and/or protein, or a combination or variant thereof.

In some embodiments, the AAV particle of the disclosure (e.g., an AAV particle comprising an AAV capsid variant) is useful for the treatment, prophylaxis, palliation or amelioration of Huntington's Disease. In some embodiments, treatment of ALS comprises the use of an AAV particle described herein (e.g., an AAV particle comprising an AAV capsid variant described herein) for a gene knock-down (e.g., knock-out) therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an HTT gene, mRNA, and/or protein, or a variant thereof.

In some embodiments, the AAV particle of the disclosure (e.g., an AAV particle comprising an AAV capsid variant) is useful for the treatment, prophylaxis, palliation or amelioration of spinal muscular atrophy. In some embodiments, treatment of spinal muscular atrophy comprises the use of an AAV particle described herein (e.g., an AAV particle comprising an AAV capsid variant described herein) for a gene replacement therapy. In some embodiments, the payload encoded by an AAV particle comprising a capsid variant described herein comprises an SMN1 protein, an SMN2 protein, or a combination or variant thereof.

In some embodiments, the AAV particle of the disclosure (e.g., an AAV particle comprising an AAV capsid variant) is useful for the treatment, prophylaxis, palliation or amelioration of multiple system atrophy. In some embodiments, treatment of multiple system atrophy comprises the use of an AAV particle described herein (e.g., an AAV particle comprising an AAV capsid variant described herein) for a gene replacement therapy.

In some embodiments, the AAV particle of the disclosure (e.g., an AAV particle comprising an AAV capsid variant) is useful for the treatment, prophylaxis, palliation or amelioration of Gaucher disease (GD) (e.g., Type 1 GD, Type 2 GD, or Type 3 GD). In some embodiments, the AAV particle of the disclosure (e.g., an AAV particle comprising an AAV capsid variant) is useful for the treatment, prophylaxis, palliation or amelioration of Parkinson's disease associated with a GBA mutation. In some embodiments, the AAV particle of the disclosure (e.g., an AAV particle comprising an AAV capsid variant) is useful for the treatment, prophylaxis, palliation or amelioration of dementia with Lewy Bodies (DLB).

In some embodiments, the AAV particle of the disclosure (e.g., an AAV particle comprising an AAV capsid variant) is useful for treatment, prophylaxis, palliation or amelioration of a leukodystrophy, e.g., Alexander disease, autosomal dominant leukodystrophy with autonomic diseases (ADLD), adrenoleukodystrophy (ALD), Canavan disease, cerebrotendinous xanthomatosis (CTX), metachromatic leukodystrophy (MLD), Pelizaeus-Merzbacher disease, or Refsum disease. In some embodiments, treatment of MLD comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ARSA protein or variant thereof. In some embodiments, treatment of ALD comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ABCD-1 protein or variant thereof.

In some embodiments, the AAV particle of the disclosure (e.g., an AAV particle comprising an AAV capsid variant) is useful for the treatment, prophylaxis, palliation, or amelioration of megalencephalic leukoencephalopathy (MLC). In some embodiments, treatment of MLC comprises the use of an AAV particle described herein (e.g., an AAV particle comprising an AAV capsid variant described herein) for a gene replacement therapy. In some embodiments, the payload encoded by an AAV particle comprising a capsid variant described herein comprises an MLC1 protein or variant thereof.

In some embodiments, the AAV particle of the disclosure (e.g., an AAV particle comprising an AAV capsid variant) is useful for the treatment, prophylaxis, palliation, or amelioration of Krabbe disease. In some embodiments, treatment of Krabbe disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GALC protein or variant thereof.

In some embodiments, the AAV particle of the disclosure (e.g., an AAV particle comprising an AAV capsid variant) is useful for the treatment, prophylaxis, palliation, or amelioration of Mucopolysaccharidosis, e.g., a Type I (MPS I), Type II (MPS II), Type IIIA (MPS IIIA), Type IIIB (MPS IIIB), or Type IIIC (MPS IIIC). In some embodiments, treatment of Mucopolysaccharidosis comprises the use of an AAV particle described herein (e.g., an AAV particle comprising an AAV capsid variant described herein) for a gene replacement therapy or a gene editing therapy (e.g., enhancement or correction). In some embodiments, the payload encoded or corrected by an AAV particle comprising a capsid variant described herein comprises an IDUA protein, IDS protein, SGSH protein, NAGLU protein, HGSNAT protein, or a combination or variant thereof.

In some embodiments, the AAV particle of the disclosure (e.g., an AAV particle comprising an AAV capsid variant) is useful for the treatment, prophylaxis, palliation, or amelioration of Batten/NCL. In some embodiments, treatment of Batten/NCL comprises the use of an AAV particle described herein (e.g., an AAV particle comprising an AAV capsid variant described herein) for a gene replacement therapy. In some embodiments, the payload encoded by an AAV particle comprising a capsid variant described herein comprises a CLN1 protein, CLN2 protein, CLN3 protein, CLN5 protein, CLN6 protein, CLN7 protein, CLN8 protein, or a combination or variant thereof.

In some embodiments, the AAV particle of the disclosure (e.g., an AAV particle comprising an AAV capsid variant) is useful for the treatment, prophylaxis, palliation or amelioration of Rett Syndrome. In some embodiments, treatment of Rett Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises an MeCP2 protein or variant thereof.

In some embodiments, the AAV particle of the disclosure (e.g., an AAV particle comprising an AAV capsid variant) is useful for the treatment, prophylaxis, palliation, or amelioration of Angelman Syndrome. In some embodiments, treatment of Angelman Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises a UBE3A protein or variant thereof.

In some embodiments, the AAV particle of the disclosure (e.g., an AAV particle comprising an AAV capsid variant) is useful for the treatment, prophylaxis, palliation, or amelioration of Fragile X Syndrome. In some embodiments, treatment of Fragile X Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises a Reelin protein, a DgkK protein, a FMR1 protein, or a combination or variant thereof.

In some embodiments, the AAV particle of the disclosure (e.g., an AAV particle comprising an AAV capsid variant) is useful for the treatment, prophylaxis, palliation, or amelioration of Canavan Disease. In some embodiments, treatment of Canavan Disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ASPA protein or variant thereof.

In some embodiments, the AAV particle of the disclosure (e.g., an AAV particle comprising an AAV capsid variant) is useful for the treatment, prophylaxis, palliation, or amelioration of a Gangliosidosis, e.g., a GM1 Gangliosidosis or a GM2 Gangliosidosis (e.g., Tay Sachs Sandhoff). In some embodiments, treatment of a Gangliosidosis, e.g., a GM1 Gangliosidosis or a GM2 Gangliosidosis (e.g., Tay Sachs Sandhoff), comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GLB1 protein, a HEXA protein, a HEXB protein, a GM2A protein, or a combination or variant thereof.

In some embodiments, the AAV particle of the disclosure (e.g., an AAV particle comprising an AAV capsid variant) is useful for the treatment, prophylaxis, palliation, or amelioration of GM3 Synthase Deficiency. In some embodiments, treatment of GM3 Synthase Deficiency comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ST3GAL5 protein or variant thereof.

In some embodiments, the AAV particle of the disclosure (e.g., an AAV particle comprising an AAV capsid variant) is useful for the treatment, prophylaxis, palliation, or amelioration of a Niemann-Pick disorder, e.g., a Niemann-Pick A or a Niemann-Pick C1 (NPC-1). In some embodiments, treatment of a Niemann-Pick disorder, e.g., a Niemann-Pick A or a Niemann-Pick C1 (NPC-1) comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ASM protein, an NPC1 protein, or variant thereof.

In some embodiments, the AAV particle of the disclosure (e.g., an AAV particle comprising an AAV capsid variant) is useful for the treatment, prophylaxis, palliation, or amelioration of Schwannoma (e.g., Neuroma). In some embodiments, treatment of Schwannoma (e.g., Neuroma) comprises the use of an AAV particle described herein (e.g., an AAV particle comprising an AAV capsid variant described herein) for a gene replacement therapy. In some embodiments, the payload encoded by an AAV particle comprising a capsid variant described herein comprises a Caspase-1 protein or variant thereof.

In some embodiments, the AAV particle of the disclosure (e.g., an AAV particle comprising an AAV capsid variant) is useful for the treatment, prophylaxis, palliation, or amelioration of a Tuberous Sclerosis, e.g., Tuberous Sclerosis Type 1 or Tuberous Sclerosis Type 2. In some embodiments, treatment of Tuberous Sclerosis, e.g., Tuberous Sclerosis Type 1 or Tuberous Sclerosis Type 2 comprises the use of an AAV particle described herein (e.g., an AAV particle comprising an AAV capsid variant described herein) for a gene replacement therapy. In some embodiments, the payload encoded by an AAV particle comprising a capsid variant described herein comprises a TSC1 protein, a TSC2 protein, or variant thereof.

In some embodiments, the AAV particle of the disclosure (e.g., an AAV particle comprising an AAV capsid variant) is useful for the treatment, prophylaxis, palliation, or amelioration of a CDKL5 Deficiency. In some embodiments, treatment of a CDKL5 Deficiency comprises the use of an AAV particle described herein (e.g., an AAV particle comprising an AAV capsid variant described herein) for a gene replacement therapy. In some embodiments, the payload encoded by an AAV particle comprising a capsid variant described herein comprises a CDKL5 protein or variant thereof.

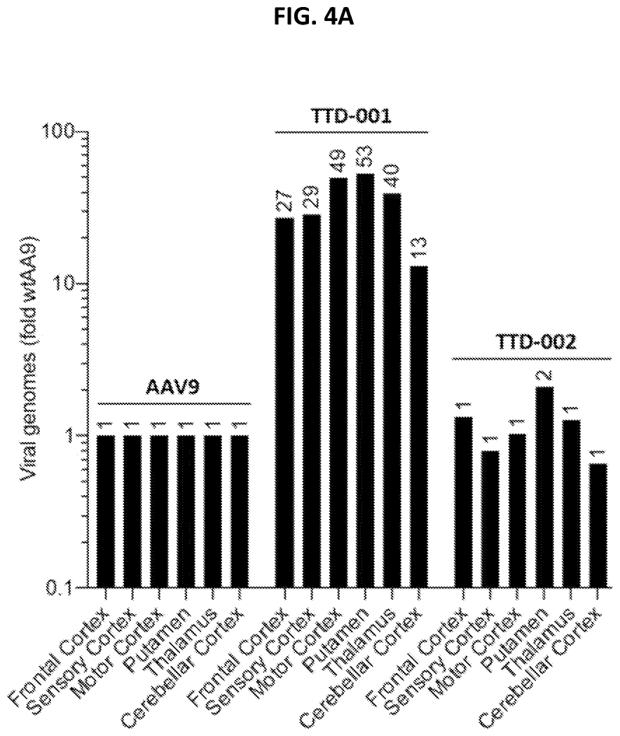

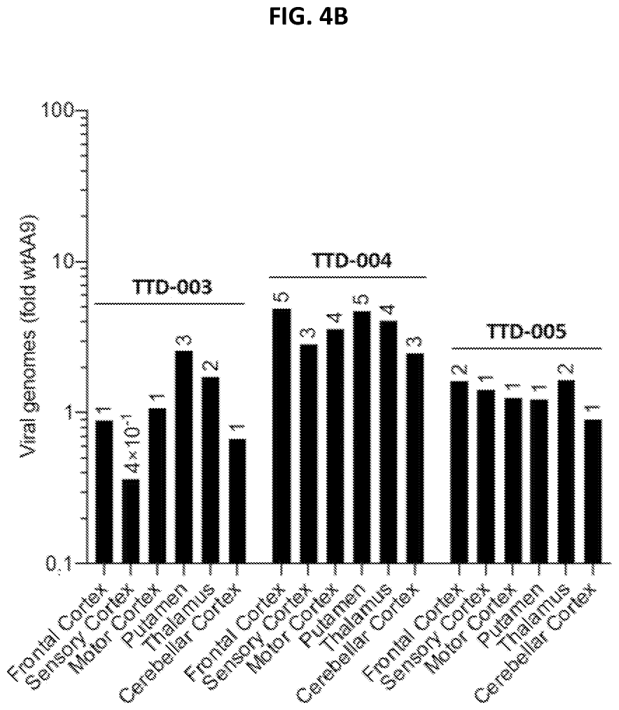

In some embodiments, the AAV particle of the disclosure (e.g., an AAV particle comprising an AAV capsid variant) is useful for the treatment, prophylaxis, palliation, or amelioration of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder. In some embodiments, treatment of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder, comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GJB1 protein, a MFN2 protein, a FIG. 4 protein, or variant thereof.

In some embodiments, the AAV particle of the disclosure (e.g., an AAV particle comprising an AAV capsid variant) is useful for the treatment, prophylaxis, palliation, or amelioration of an Aspartylglucosaminuria (AGU). In some embodiments, treatment of an AGU comprises the use of an AAV particle described herein (e.g., an AAV particle comprising an AAV capsid variant described herein) for a gene replacement therapy. In some embodiments, the payload encoded by an AAV particle comprising a capsid variant described herein comprises an AGA protein or variant thereof.

In some embodiments, the AAV particle of the disclosure (e.g., an AAV particle comprising an AAV capsid variant) is useful for the treatment, prophylaxis, palliation, or amelioration of a Leigh Syndrome. In some embodiments, treatment of a Leigh Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises a SURF1 protein or variant thereof.

In some embodiments, the AAV particle of the disclosure (e.g., an AAV particle comprising an AAV capsid variant) is useful for the treatment, prophylaxis, palliation, or amelioration of epilepsy. In some embodiments, treatment of epilepsy comprises the use of an AAV particle described herein (e.g., an AAV particle comprising an AAV capsid variant described herein) for a gene replacement therapy. In some embodiments, the payload encoded by an AAV particle comprising a capsid variant described herein comprises an NPY/Y2 protein, a Galanin protein, a Dynorphin protein, an AIMP2-DX2 protein, an SLC6A1 protein, an SLC13A5 protein, a KCNQ2 protein, or variant thereof.

In some embodiments, the AAV particle of the disclosure (e.g., an AAV particle comprising an AAV capsid variant) is useful for the treatment, prophylaxis, palliation, or amelioration of a Dravet Syndrome. In some embodiments, treatment of Dravet Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises an SCN1a protein, or variant thereof.

In some embodiments, the AAV particle of the disclosure (e.g., an AAV particle comprising an AAV capsid variant) is useful for the treatment, prophylaxis, palliation, or amelioration of a Duchenne muscular dystrophy (DMD). In some embodiments, treatment of DMD comprises the use of an AAV particle described herein (e.g., an AAV particle comprising an AAV capsid variant described herein) for a gene replacement therapy or enhancement (e.g., correction of exon-skipping), or a gene editing therapy (e.g., enhancement or correction). In some embodiments, the payload encoded or corrected by an AAV particle comprising a capsid variant described herein comprises a Dystrophin gene and/or protein, a Utrophin gene and/or protein, or a GALGT2 gene and/or protein, or a Follistatin gene and/or protein, or a combination or variant thereof.

In some embodiments, the AAV particle of the disclosure (e.g., an AAV particle comprising an AAV capsid variant) is useful for the treatment, prophylaxis, palliation, or amelioration of Pompe Disease. In some embodiments, treatment of Pompe Disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GAA protein, or variant thereof.

In some embodiments, the AAV particle of the disclosure (e.g., an AAV particle comprising an AAV capsid variant) is useful for the treatment, prophylaxis, palliation, or amelioration of Limb-Girdle Muscular Dystrophy (LGMD2A). In some embodiments, treatment of LGMD2A comprises the use of an AAV particle described herein (e.g., an AAV particle comprising an AAV capsid variant described herein) for a gene replacement therapy. In some embodiments, the payload encoded by an AAV particle comprising a capsid variant described herein comprises a CAPN-3 protein, DYSF protein, a SGCG protein, a SGCA protein, a SGCB protein, a FKRP protein, a ANO5 protein, or a combination or variant thereof.

In some embodiments, the AAV particle of the disclosure (e.g., an AAV particle comprising an AAV capsid variant) is useful for the treatment, prophylaxis, palliation, or amelioration of chronic or neuropathic pain.

In some embodiments, the AAV particle of the disclosure (e.g., an AAV particle comprising AAV capsid variant) is useful for treatment, prophylaxis, palliation, or amelioration of a disease associated with the central nervous system.

In some embodiments, the AAV particle of the disclosure (e.g., an AAV particle comprising an AAV capsid variant) is useful for treatment, prophylaxis, palliation, or amelioration of a disease associated with the peripheral nervous system.

In some embodiments, the AAV particle of the disclosure (e.g., an AAV particle comprising an AAV capsid variant) is useful for treatment, prophylaxis, palliation, or amelioration of a neuro-oncological disorder in a subject. In some embodiments, treatment of a neuro-oncological disorder comprises prevention of said neuro-oncological disorder. In some embodiments, a neuro-oncological disorder comprises a cancer of a primary CNS origin (e.g., a CNS cell, a tissue, or a region), or a metastatic cancer in a CNS cell, tissue, or region. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, the AAV particle of the disclosure (e.g., an AAV particle comprising an AAV capsid variant) is useful for the treatment, prophylaxis, palliation, or amelioration of a disease associated with expression of HER2, e.g., a disease associated with overexpression of HER2. In some embodiments, the AAV particle of the disclosure (e.g., an AAV particle comprising an AAV capsid variant) is useful for the treatment, prophylaxis, palliation, or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, the AAV particle of the disclosure (e.g., an AAV particle comprising an AAV capsid variant) is useful for treatment, prophylaxis, palliation, or amelioration of a muscular disorder and/or neuromuscular disorder in a subject. In some embodiments, treatment of a muscular disorder and/or neuromuscular disorder comprises prevention of said muscular disorder and/or neuromuscular disorder.

In some embodiments, the AAV particle of the disclosure (e.g., an AAV particle comprising an AAV capsid variant) is useful for treatment, prophylaxis, palliation or amelioration of a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction. In some embodiments, the cardiac disease is a disease associated with expression, e.g., aberrant expression, of LAMP2B, MYBPC3, TNNI3, LMNA, BAG3, DWORF, PKP2, Cx43, TAZ, CASQ2, SERCA2a, I-1c, S100A1 and/or ARC, S100A1, ASCL1, miR133, Mydelta3, Sav, or a combination or variant thereof. In some embodiments, treatment of a cardiac disorder described herein comprises the use of an AAV particle described herein (e.g., an AAV particle comprising an AAV capsid variant described herein) for a gene replacement therapy.

In some embodiments, the cardiac disease is a genetic disorder, e.g., an autosomal dominant genetic disorder, an autosomal recessive disorder, or an X-linked recessive genetic disorder. In some embodiments, the cardiomyopathy is a genetic disorder, e.g., a genetic disorder associated with an abnormality (e.g., mutation, insertion, rearrangement and/or deletion) in a gene chosen from TTN, LMNA, MYH7, MYH6, SCN5A, TNNT2, RBM20, TNNI3, MYL2, MYL3, PKP2, DSP, DSG2, DSC2, JUP, or a combination thereof. In some embodiments, the cardiac disorder is a dilated cardiomyopathy, e.g., a dilated cardiomyopathy associated with an abnormality (e.g., mutation, insertion, rearrangement and/or deletion) in a gene chosen from TTN, LMNA, MIH7, BAG3, MIPN, TNNT2, SCN5A, RBN20, TNPO, LAMA4, VCL, LDB3, TCAP, PSEN1/2, ACTN2, CRYAB, TPM1, ABCC9, ACTC1, PDLIM3, ILK, TNNC1, TNNI3, PLN, DES, SGCD, CSRP3, MIH6, EYA4, ANKRD1, DMD, GATAD1, TAZ/G4.5, or combination thereof. In some embodiments, the cardiac disorder is a hypertrophic cardiomyopathy, e.g., a hypertrophic cardiomyopathy associated with an abnormality (e.g., mutation, insertion, rearrangement and/or deletion) in a gene chosen from MYH7, TNNT2, TNNI3, TPM1, MYL2, MYL3, ACTC1, CSRP3, TTN, ACTN2, MYH6, TCAP, TNNC1, or a combination thereof. In some embodiments, the cardiac disorder is an arrhythmogenic ventricular cardiomyopathy, e.g., an arrhythmogenic ventricular cardiomyopathy associated with an abnormality (e.g., mutation, insertion, rearrangement and/or deletion) in a gene chosen from PKP2, DSG2, DSP, RYR2, DSC2, TGFB3, TMEM43, DES, TTN, LMNA, or a combination thereof.

In some embodiments, the AAV particle of the present disclosure (e.g., an AAV particle comprising an AAV capsid polypeptide, e.g., an AAV capsid variant) is administered to a subject having at least one of the diseases or symptoms described herein. In some embodiments, an AAV particle of the present disclosure is administered to a subject having or diagnosed with having a disease or disorder described herein.

Any neurological disease or disorder, neurodegenerative disorder, muscular disorder, neuromuscular disorder, and/or neuro-oncological disorder may be treated with the AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy—Congenital, Myopathy—Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

### Pharmaceutical Composition and Formulations

According to the present disclosure, an AAV particle comprising an AAV capsid variant described herein may be prepared as a pharmaceutical composition. In some embodiments, the pharmaceutical composition comprises at least one active ingredients. In some embodiments, the pharmaceutical composition comprises a pharmaceutically acceptable excipient.

In some embodiments, an AAV particle of the present disclosure (e.g., an AAV particle comprising an AAV capsid polypeptide, e.g., an AAV capsid variant) can be formulated using an excipient to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload. Formulations of the present disclosure can include, without limitation, saline, liposomes, lipid nanoparticles, polymers, peptides, proteins, cells transfected with viral vectors (e.g., for transfer or transplantation into a subject) and combinations thereof.

In some embodiments, the relative amount of the active ingredient (e.g. an AAV particle comprising an AAV capsid variant described herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the pharmaceutical composition comprising an AAV particle described herein may comprise an AAV capsid variant and a viral genome encoding a payload, e.g., a payload described herein, with or without a pharmaceutically acceptable excipient.

The present disclosure also provides in some embodiments, a pharmaceutical composition suitable for administration to a subject, e.g., a human. In some embodiments, the pharmaceutical composition is administered to a subject, e.g., a human.

Also provided herein are formulations, e.g., optimized stable formulations for AAV particles and variants thereof, e.g., comprising an AAV capsid variant described herein (e.g., an AAV capsid variant comprising an amino acid sequence described herein, e.g., in any one of Tables 1A, 1B, 2-7, 10-11, or 20, or the amino acid sequence of SEQ ID NO: 3636, or a variant thereof). Without wishing to be bound by theory, it is believed in some embodiments, that such formulations are advantageous over conventional formulations (e.g., PBS-based formulations), for example, in terms of stability under various storage conditions, the ability to support viral concentrations >1×10¹³ vg/ml, and having desirable characteristics over a wide range of parameters, such as pH, pI, osmolality, osmolarity, occupancy (e.g., % full capsids), and aggregation.

In some embodiments, the formulation comprises a buffering agent. Non-limiting examples of buffering agents include, e.g., Tris base, Tris Hcl, Bis-tris propane (BTP), phosphate-buffered saline (PBS), sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), and MOPS (3-(N-morpholino)propanesulfonic acid). In some embodiments, the buffering agent is Tris.

In some embodiments the formulation comprises a salt. Non-limiting examples of salts include, e.g., sodium chloride, potassium chloride, magnesium chloride. In some embodiments, the salt is sodium chloride.

In some embodiments, the formulation comprises a polyether, e.g., a polyether with low molecular weight (e.g., 200-500)). Non-limiting examples of polyethers include, e.g., glycerol, glycerin, and PEG (e.g., low-molecular-weight PEG, e.g., PEG-300). In some embodiments, the polyether is glycerol. In some embodiments, the polyether is PEG (e.g., low-molecular-weight PEG, e.g., PEG-300).

In some embodiments, at least one of the components of the formulation is a sugar, such as a disaccharide, or a sugar substitute. Non-limiting examples of sugars (e.g., disaccharide sugars) include trehalose, sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, saccharose, and xylobiose. A non-limiting example of a sugar substitute is sorbitol. In some embodiments, the sugar is trehalose.

In some embodiments, the formulation comprises a surfactant, for example, an anionic, zwitterionic, or non-ionic surfactant. Non-limiting examples of anionic surfactants include, e.g., sulfate, sulfonate, phosphate esters, and carboxylates. Non-limiting examples of nonionic surfactants include, e.g., ehoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., poloxamers such as Pluronic® F-68 or F-127), esters of fatty acids and polyhydric alcohols, fatty acid alkanolamides, ethoxylated aliphatic acids, ethoxylated aliphatic alcohols, ethoxylated sorbitol fatty acid esters, ethoxylated glycerides, ethoxylated block copolymers with EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants. Non-limiting examples of zwitterionic surfactants include, e.g., alkylamido betaines and amine oxides thereof, alkyl betaines and amine oxides thereof, sulfo betaines, hydroxy sulfo betaines, amphoglycinates, amphopropionates, balanced amphopolycarboxyglycinates, and alkyl polyaminoglycinates. In some embodiments, the surfactant is a poloxamer, e.g., Pluronic® F-68 or F-127. In some embodiments, the surfactant is Pluronic® F-68.

In some embodiments, the formulation comprises (a) an AAV particle or variant thereof described herein, e.g., an AAV particle comprising an AAV capsid variant described herein, (b) a buffering agent (e.g., Tris), and (c) a polyether (e.g., glycerol).

In some embodiments, the formulation comprises (a) an AAV particle or variant thereof described herein, e.g., an AAV particle comprising an AAV capsid variant described herein, (b) a buffering agent (e.g., Tris), and (c) a sugar (e.g., trehalose).

In some embodiments, the formulation comprises (a) an AAV particle or variant thereof described herein, e.g., an AAV particle comprising an AAV capsid variant described herein, (b) a buffering agents (e.g., Tris), (c) a polyether (e.g., glycerol), and (d) a salt (e.g., sodium chloride).

In some embodiments, the formulation comprises (a) an AAV particle or variant thereof as described herein, e.g., an AAV particle comprising an AAV capsid variant described herein, (b) a buffering agent (e.g., Tris), (c) a sugar (e.g., trehalose), and (d) a salt (e.g., sodium chloride).

In some embodiments, the formulation comprises (a) an AAV particle or variant thereof, e.g., an AAV particle comprising an AAV capsid variant described herein, (b) a buffering agent (e.g., Tris), (c) a polyether (e.g., glycerol), and (d) a surfactant (e.g., ethylene oxide/propylene oxide copolymer).

In some embodiments, the formulation comprises (a) an AAV particle or variant thereof, e.g., an AAV particle comprising an AAV capsid variant described herein, (b) a buffering agent (e.g., Tris), (c) a sugar (e.g., trehalose), and (d) a surfactant (e.g., ethylene oxide/propylene oxide copolymer).

In some embodiments, the formulation comprises (a) an AAV particle or variant thereof, e.g., an AAV particle comprising an AAV capsid variant described herein, (b) a buffering agent (e.g., Tris), (c) a salt (e.g., sodium chloride), (d) a polyether (e.g., glycerol), and (e) a surfactant (e.g., ethylene oxide/propylene oxide copolymer).

In some embodiments, the formulation comprises (a) an AAV particle or variant thereof, e.g., an AAV particle comprising an AAV capsid variant described herein, (b) a buffering agent (e.g., Tris), (c) a salt (e.g., sodium chloride), (d) a sugar (e.g., trehalose), and (e) a surfactant (e.g., ethylene oxide/propylene oxide copolymer).

In some embodiments, the formulation is an aqueous formulation. In some embodiments, the formulation is an isotonic solution (e.g., a solution comprising an osmolarity of about 270-310 mOsm/L). In some embodiments, the formulation comprises an osmolarity greater than 310 mOsm/L.

In some embodiments, the buffering agent in a formulation described herein is Tris. The buffering agent is a weak acid or base that, when used in the formulation, maintains the pH of the formulation near a chosen value even after another acid or base is added to the formulation. In some embodiments, the buffering agent is capable of maintaining a pH of 7.8-8.4 (e.g., 7.8, 7.9, 8.0, 8.1, 8.2, 8.3, or 8.4). In some embodiments, buffering agent (e.g., Tris) is present at a concentration of between 1-50 mM, for example, about 1 mM, about 5 mM, about 10 mM, about 15 mM, about 20 mM, about 25 mM, about 30 mM, about 35 mM, about 40 mM, about 50 mM, 1-40 mM, 1-30 mM, 1-20 mM, 1-10 mM, 5-40 mM, 5-35 mM, 5-30 mM, 5-25 mM, 5-20 mM, 5-15 mM, 5-10 mM, 10-40 mM, 10-35 mM, 10-30 mM, 10-25 mM, 10-20 mM, 10-15 mM, 15-40 mM, 15-35 mM, 15-30 mM, 15-25 mM, 15-20 mM, 20-50 mM, 20-40 mM, 20-35 mM, 20-30 mM, 20-25 mM, 25-50 mM, 25-40 mM, 25-35 mM, 25-30 mM, 30-50 mM, 30-40 mM, 30-35 mM, 40-50 mM, 45-50 mM. In some embodiments, the formulation comprises Tris at a concentration of 15-25 mM, e.g., 20 mM Tris or about 20 mM Tris.

In some embodiments, the salt in a formulation described herein is sodium chloride. In some embodiments, the salt (e.g., sodium chloride) is present at a concentration of between 30-80 mM, for example, about 40 mM, about 45 mM, about 50 mM, about 55 mM, about 60 mM, about 65 mM, about 70 mM, about 75 mM, about 80 mM, 40-80 mM, 50-80 mM, 60-80 mM, 70-80 mM, 40-70 mM, 50-70 mM, 60-70 mM, 40-65 mM, 50-65 mM or 60-65 mM. In some embodiments, the salt is present at a concentration between 70-135 mM, e.g., 70-90 mM or 70-100 mM. In some embodiments, the formulation comprises sodium chloride at a concentration of 60-65 mM, e.g., 62.5 mM sodium chloride or about 62.5 mM sodium chloride.

In some embodiments, the polyether in a formulation described herein is glycerol. In some embodiments, the polyether (e.g., glycerol) is present at a concentration of between 0.25%-5%, for example, about 0.25%, about 0.5%, about 1%, about 1.5%, about 2.0%, about 2.5%, about 3.0%, about 3.5%, about 4.0%, about 4.5%, about 5%, 0.5-5.0%, 1.0-5.0%, 1.5%-5.0%, 2.0%-5.0%, 2.5%-5.0%, 3.0-5.0%, 3.5-5.0%, 4.0-5.0%, 4.5-5.0%, 0.25-4.0%, 0.5-4.0%, 1.0-4.0%, 1.5-4.0%, 2.0-4.0%, 2.5-4.0%, 3.0-4.0%, 3.5-4.0%, 0.25-3.5%, 0.5-3.5%, 1.0-3.5%, 1.5-3.5%, 2.0-3.5%, 2.5-3.5%, 3.0-3.5%, 0.25-3.0%, 0.5-3.0%, 1.0-3.0%, 1.5-3.0%, 2.0-3.0%, 2.5-3.0%, 0.25-2.5%, 0.5-2.5%, 1.0-2.5%, 1.5-2.5%, 2.0-2.5%, 0.25-2.0%, 0.5-2.0%, 1.0-2.0%, 1.5-2.0%, 0.25-1.5%, 0.5-1.5%, 1.0-1.5%, 0.25-1.0%, 0.5-1.0%, 0.75-3.0%, 0.75-1.25%, or 2.25-2.75%. In some embodiments, the polyether is present at a concentration of about 3-7.5%, e.g., about 3-7%, 4-7%, 4.5-7.5%, 4.5-7%, 5-7.5%, 5-7%, 5.5-7.5%, 5.5-7%, 6-7.5%, 6-7%, 6.5-7%, 6.5-7.5%, 5-6%, 5-6.5%, 5.5-6.5%, 5.5-6%, 4-6.5%, 4-6%, 3-5%, 3-5.5%, 3-6%, 3-6.5%, 3-4%, 3.5-7%, or 3.5-7.5%. In some embodiments, the formulation comprises glycerol at a concentration of about 0.75-1.25%, 2.25-2.75%, or 3-5%. In some embodiments, the formulation comprises glycerol at a concentration of 1% or about 1%. In some embodiments, the formulation comprises glycerol at a concentration of 2.5% or about 2.5%. In some embodiments, the polyether in the formulation is glycerin.

In some embodiments, the polyether in a formulation described herein is polyethylene glycol (PEG). In some embodiments, the PEG is low molecular weight PEG. In some embodiments, the PEG has a molecular weight ≤300, for example, ≤290, ≤280, ≤270, ≤260, ≤250, ≤240, ≤230, ≤220, ≤210, ≤200, 200-500, 250-500, 300-500, 350-500, 400-500, 450-500, 200-450, 250-450, 300-450, 350-450, 400-450, 200-400, 250-400, 300-400, 350-400, 200-350, 250-350, 300-350, 200-300, 250-300, 275-325, or 290-310. In some embodiments, the formulation comprises PEG having a molecular weight of 275-325. In some embodiments, the formulation comprises PEG having a molecular weight of 300 or about 300 (e.g., PEG-300).

In some embodiments, the sugar in a formulation described herein is trehalose. In some embodiments, the sugar (e.g., trehalose) is present at a concentration of between 3-9%, for example, about 3%, about 3.5%, about 4.0%, about 4.5%, about 5%, about 5.5%, about 6%, about 6.5%, about 7.0%, about 7.5%, about 8.0%, about 8.5%, about 9.0%, 3.5-9.0%, 4.0-9.0%, 4.5-9.0%, 5.0-9.0%, 5.5-9.0%, 6.0-9.0%, 6.5-9.0%, 7.0-9.0%, 7.5-9.0%, 8.0-9.0%, 8.5-9.0%, 3.0-8.0%, 3.5-8.0%, 4.0-8.0%, 4.5-8.0%, 5.0-8.0%, 5.5-8.0%, 6.0-8.0%, 6.5-8.0%, 7.0-8.0%, 7.5-8.0%, 3.0-7.5%, 3.5-7.5%, 4.0-7.5%, 4.5-7.5%, 5.0-7.5%, 5.5-7.5%, 6.0-7.5%, 6.5-7.5%, 7.0-7.5%, 3.0-7.0%, 3.5-7.0%, 4.0-7.0%, 4.5-7.0%, 5.0-7.0%, 5.5-7.0%, 6.0-7.0%, 6.5-7.0%, 3.0-6.5%, 3.5-6.5%, 4.0-6.5%, 4.5-6.5%, 5.0-6.5%, 5.5-6.5%, 6.0-6.5%, 3.0-6.0%, 3.5-6.0%, 4.0-6.0%, 4.5-6.0%, 5.0-6.0%, 5.5-6.0%, 3.0-5.5%, 3.5-5.5%, 4.0-5.5%, 4.5-5.5%, 5.0-5.5%, 3.0-5.0%, 3.5-5.0%, 4.0-5.0%, 4.5-5.0%, 3.0-4.5%, 3.5-4.5%, 3.0-4.5%, 3.0-4.0%, 3.5-4.0%, 5.75-6.25, 5.8-6.2, or 5.9-6.1. In some embodiments, the formulation comprises trehalose at a concentration of 5.8-6.2%. In some embodiments, the formulation comprises trehalose at a concentration of 5.95% or about 5.95%.

In some embodiments, a formulation described herein comprises a sugar, e.g., trehalose, at the same overall molecular weight (Da) by density (g/cm³), e.g., assuming saturated equivalence to that of 100% glycerol, v/v. In some embodiments, the density of glycerol in the formulation is 1.26 g/cm³ (e.g., 1.60% equivalent). In some embodiments, the density of trehalose in the formulation is 1.58 g/cm³ (e.g., 5.95% equivalent).

In some embodiments, the surfactant in a formulation described herein is Pluronic F68. In some embodiments, the surfactant (e.g., an ethylene oxide/propylene oxide copolymer such as a poloxamer (e.g., Pluronic F68)) is present at a concentration (w/v) of between 0.0002-0.002%, for example, 0.0004-0.002%, 0.0006-0.002%, 0.0008-0.002%, 0.001-0.002%, 0.0012-0.002%, 0.0014-0.002%, 0.0016-0.002%, 0.0018-0.002%, 0.0002-0.0018%, 0.0004-0.0018%, 0.0006-0.0018%, 0.0008-0.0018%, 0.001-0.0018%, 0.0012-0.0018%, 0.0014-0.0018%, 0.0016-0.0018%, 0.0002-0.0016%, 0.0004-0.0016%, 0.0006-0.0016%, 0.0008-0.0016%, 0.001-0.0016%, 0.0012-0.0016%, 0.0014-0.0016%, 0.0002-0.0014%, 0.0004-0.0014%, 0.0006-0.0014%, 0.0008-0.0014%, 0.001-0.0014%, 0.0012-0.0014%, 0.0002-0.0012%, 0.0004-0.0012%, 0.0006-0.0012%, 0.0008-0.0012%, 0.001-0.0012%, 0.0002-0.0010%, 0.0004-0.0010%, 0.0006-0.0010%, 0.0008-0.0010%, 0.0002-0.0008%, 0.0004-0.0008%, 0.0006-0.0008%, 0.0002-0.0006%, 0.0004-0.0006%, 0.0002-0.0004%, or 0.0009-0.0011. In some embodiments, the formulation comprises Pluronic F68 at a concentration of 0.0008-0.0012%. In some embodiments, the formulation comprises Pluronic F68 at a concentration of 0.001% or about 0.001%.

In some embodiments, the formulations provided herein exhibits one, two, three, four, five, six, seven, or all of the following properties: (a) a pH in the range of between 6-9, for example, a pH of about 6.0, about 6.2, about 6.4, about 6.6, about 6.8, about 7.0, about 7.2, about 7.4, about 7.6, about 7.8, about 8.0, about 8.1, about 8.2, about 8.3, about 8.4, about 8.5, about 8.6, about 8.7, about 8.8, about 8.9, about 9.0, 6.0-8.5, 6.0-7.5, 6.0-7.0, 6.0-6.5, 6.5-9.0, 6.5-8.5, 6.5-8.0, 6.5-7.5, 6.5-7.0, 7.0-9.0, 7.0-8.5, 7.0-8.0, 7.0-7.5, 7.5-9.0, 7.5-8.5, 7.5-8.0, 8.0-9.0, 8.0-8.9, 8.0-8.8, 8.0-8.7, 8.0-8.6, 8.0-8.5, 8.0-8.4, 8.0-8.3, 8.0-8.2, 8.0-8.1, 7.8-8.5, 7.9-8.3, or 7.9-8.2, as assessed by, e.g., a pH meter, as described in Example 9; (b) an osmolality (mOsm/kg) in the range of between 250-650, for example, 250-650, 250-600, 250-550, 250-500, 250-450, 250-400, 250-350, 250-300, 300-650, 300-600, 300-550, 300-500, 300-450, 300-400, 300-350, 350-650, 350-600, 350-550, 350-500, 350-450, 350-400, 400-650, 400-600, 400-550, 400-500, 400-450, 450-650, 450-600, 450-550, 450-500, 500-650, 500-600, 500-550, 550-650, 550-600, 310-400, 320-400, 330-400, 340-400, 350-400, 360-400, 370-400, 380-400, 390-400, 300-390, 300-380, 300-370, 300-360, 300-350, 300-340, 300-330, 300-320, or 300-310, as assessed by, e.g., an osmometer as described in Example 9. as assessed by, e.g., an osmometer as described in Example 9; (c) a viral titer (e.g., TTD-001 titer)>1×10¹² vg/ml, for example, >2×10¹² vg/ml, >4×10¹² vg/ml, >6×10¹² vg/ml, >8×10¹² vg/ml, >1.0×10¹³ vg/ml, >1.5×10¹³ vg/ml, >2.0×10¹³ vg/ml, >2.5×10¹³ vg/ml, >3.0×10¹³ vg/ml, >3.5×10¹³ vg/ml, >4.0×10¹³ vg/ml, >4.5×10¹³ vg/ml, >5.0×10¹³ vg/ml, >5.5×10¹³ vg/ml, >6.0×10¹³ vg/ml, >6.5×10¹³ vg/ml, >7.0×10¹³ vg/ml, >7.5×10¹³ vg/ml, >8.0×10¹³ vg/ml, >8.5×10¹³ vg/ml, >9.0×10¹³ vg/ml, >9.5×10¹³ vg/ml, >1.0×10¹⁴ vg/ml, 1.0×10¹²-1.0×10¹⁴ vg/ml, 2×10¹²-1.0×10¹⁴ vg/ml, 4.0×10¹²-1.0×10¹⁴ vg/ml, 6.0×10¹²-1.0×10¹⁴ vg/ml, 8.0×10¹²-1.0×10¹⁴ vg/ml, 1.0×10¹³-1.0×10¹⁴ vg/ml, 1.5×10¹³-1.0×10¹⁴ vg/ml, 2.0×10¹³-1.0×10¹⁴ vg/ml, 2.5×10¹³-1.0×10¹⁴ vg/ml, 3.0×10¹³-1.0×10¹⁴ vg/ml, 3.5×10¹³-1.0×10¹⁴ vg/ml, 4.0×10¹³-1.0×10¹⁴ vg/ml, 4.5×10¹³-1.0×10¹⁴ vg/ml, 5.0×10¹³-1.0×10¹⁴ vg/ml, 6.0×10¹³-1.0×10¹⁴ vg/ml, 7.0×10¹³-1.0×10¹⁴ vg/ml, 8.0×10¹³-1.0×10¹⁴ vg/ml, 9.0×10¹³-1.0×10¹⁴ vg/ml, 1.0×10¹³-9.0×10¹³ vg/ml, 1.0×10¹³-8.0×10¹³ vg/ml, 1.0×10¹³-7.0×10¹³ vg/ml, 1.0×10¹³-6.0×10¹³ vg/ml, 1.0×10¹³-5.0×10¹³ vg/ml, 1.0×10¹³-4.5×10¹³ vg/ml, 1.0×10¹³-4.0×10¹³ vg/ml, 1.0×10¹³-3.5×10¹³ vg/ml, 1.0×10¹³-3.0×10¹³ vg/ml, 1.0×10¹³-2.5×10¹³ vg/ml, 1.0×10¹³-2.0×10¹³ vg/ml, or 1.0×10¹³-1.5×10¹³ vg/ml, as assessed by, e.g., qPCR as described in Example 9; (d) occupancy (% full capsids)≥30%, for example, ≥35%, ≥40%, ≥45%, ≥50%, ≥55%, ≥60%, ≥65%, ≥70%, ≥75%, ≥80%, ≥85%, ≥90%, ≥95%, 30%-90%, 30%-80%, 30%-70%, 30%-65%, 30%-60%, 30%-55%, 30%-50%, 30%-45%, 30%-40%, 30%-35%, 35%-90%, 35%-80%, 35%-75%, 35%-70%, 35%-65%, 35%-60%, 35%-55%, 35%-50%, 35%-45%, 35%-40%, 40%-90%, 40%-80%, 40%-70%, 40%-65%, 40%-60%, 40%-55%, 40%-50%, 40%-45%, 45%-90%, 45%-80%, 45%-70%, 45%-65%, 45%-60%, 45%-55%, 45%-50%, 50%-90%, 50%-80%, 50%-70%, 50%-65%, 50%-60%, 50%-55%, 55%-90%, 55%-80%, 55%-70%, 55%-65%, 55%-60%, 60%-90%, 60%-80%, 60%-70%, 70%-90%, 70%-80%, or 80%-90%, as assessed by, e.g., SEC-MALS as described in Example 9; (e) aggregation≤10%, for example, ≤9%, ≤8%, ≤7%, ≤6%, ≤5%, ≤4%, ≤3%, ≤2%, ≤1%, 0-10%, 0-8%, 0-6%, 0-5%, 0-4%, 0-3%, 0-2%, 0-1%, 1-10%, 2-10%, 3-10%, 4-10%, 5-10%, 1-5%, 2-5%, or 3-5%, as assessed by % HMW using size exclusion chromatography (SEC), for example, SEC-FLD/DLS or SEC-MALS, as described in Example 9; (f) a viral titer higher than the viral titer supported by a formulation comprising PBS and 0.001% Pluronic F68 (modified PBS formulation) (e.g., higher by at least 50%, at least 100%, at least 250%, at least 500%, at least 750%, at least 1000% (e.g., one order of magnitude), at least 2000%, at least 4000%, at least 6000%, at least 8000%, at least 10,000% (e.g., two orders of magnitude), at least 25,000%, at least 50,000%, 500-50,000%, 1000-50,000%, 2500-50,000%, 5000-50,000%, 7500-50,000%, 10,000-50,000%, 25,000-50,000%, 500-25,000%, 1000-25,000%, 2500-25,000%, 5000-25,000%, 7500-25,000%, 10,000-25,000%, 500-10,000%, 1000-10,000%, 2500-10,000%, 5000-10,000%, 7500-10,000%, 500-7500%, 1000-7500%, 2500-7500%, 5000-7500%, 500-5000%, 1000-7500%, or 2500-5000%), as assessed by, e.g., qPCR as described in Example 9; (g) occupancy (% full capsids) higher than the occupancy supported by a formulation comprising PBS and 0.001% Pluronic F68 (modified PBS formulation) (e.g., higher relative to the modified PBS formulation by, e.g., ≥10%, ≥15%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90%, ≥95%, ≥100% (e.g., at least 2-times higher), ≥125%, ≥150%, 10-150%, 20-150%, 30-150%, 40-150%, 50-150%, 60-150%, 70-150%, 80-150%, 90-150%, 100-150%, 125-150%, 10-125%, 20-125%, 30-125%, 40-125%, 50-125%, 60-125%, 70-125%, 80-125%, 90-125%, 100-125%, 10-100%, 20-100%, 30-100%, 40-100%, 50-100%, 60-100%, 70-100%, 80-100%, 90-100%, 10-75%, 20-75%, 30-75%, 40-75%, 50-75%, 60-75%, 10-50%, 20-50%, 30-50%, 40-50%, or 10-25%), as assessed by, e.g., SEC-MALS as described in Example 9; (h) aggregation less than the aggregation observed with a formulation comprising PBS and 0.001% Pluronic F68 (modified PBS formulation) after multiple freeze thaw cycles (e.g., 1, 2, 3, 4, 5, 6, or more freeze thaw cycles), e.g., aggregation less than that observed with the modified PBS formulation after 1, 2, 3, 4, 5, 6, or more freeze thaw cycles by ≥20%, ≥25%, ≥30%, ≥35%, ≥40%, ≥45%, ≥50%, ≥55%, ≥60%, ≥65%, ≥70%, ≥75%, ≥80%, ≥85%, ≥90%, 20-90%, 30-90%, 40-90%, 50-90%, 60-90%, 70-90%, 80-90%, 20-80%, 30-80%, 40-80%, 50-80%, 60-80%, 70-80%, 20-70%, 30-70%, 40-70%, 50-70%, 60-70%, 20-60%, 30-60%, 40-60%, 50-60%, 20-50%, 30-50%, 40-50%, 20-40%, 30-40%, or 20-30%, as assessed by % HMW using size exclusion chromatography (SEC), for example, SEC-FLD/DLS or SEC-MALS, as described in Example 9.

In some embodiments, 1, 2, 3, 4, 5, 6, 7, or all 8 of the aforementioned properties (a)-(h) are maintained during storage (e.g., storage for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), for example, at −80° C., 2-8° C., room temperature, and/or after multiple freeze thaw cycles (e.g., 1-6 freeze thaw cycles). In some embodiments, values of the 1, 2, 3, 4, 5, 6, 7, or all 8 of the aforementioned properties (a)-(h) do not change by more than 50%, e.g., do not change by more than 45%, 40%, 35%, 30%, 25%, 20%, 15%, 10%, or 5% during storage and/or multiple freeze thaw cycles (e.g., 1, 2, 3, 4, 5, 6, or more freeze thaw cycles) relative to time 0, e.g., the formulation prior to storage and/or freeze thawing.

In some embodiments, (a) the ratio of VP3 protein to VP2 protein (VP3:VP2) of the AAV capsids of the AAV particle is about 25-35:2, about 25-34:2, about 25-33:2, about 25-32:2, about 25-30:2, about 25:2, about 26:2, about 27:2, about 28:2, about 29:2, about 30:2, about 31:2, about 32:2, about 33:2, about 34:2, or about 35:2, (b) the ratio of VP3 protein to VP1 protein (VP3:VP1) of the AAV capsids of the AAV particle is about 25-35:1 (e.g., 25-34:1, 25-33:1, 25-32:1, 25-30:1, 25:1, 26:1, 27:1, 28:1, 29:1, 30:1, 31:1, 32:1, 33:1, 34:1, or 35:1), and/or (c) the ratio of VP3 protein to VP2 and VP1 proteins (VP3:VP2:VP1) of the AAV capsids of the AAV particle is about 25-35:2:1 (e.g., 25-34:2:1, 25-33:2:1, 25-32:2:1, 25-30:2:1, 25:2:1, 26:2:1, 27:2:1, 28:2:1, 29:2:1, 30:2:1, 31:2:1, 32:2:1, 33:2:1, 34:2:1, or 35:2:1), when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by capillary gel electrophoresis-sodium dodecyl sulfate (e.g., as described in Example 9).

In some embodiments, the ratio of VP3 protein to VP2 protein (i.e., VP3:VP2), VP3 protein to VP1 protein (VP3:VP1), or VP3 protein to VP2 and VP1 proteins (VP3:VP2:VP1) of the AAV capsids of the AAV particle does not differ from a reference ratio (e.g., baseline ratio, such as the ratio at time 0) by more than about 25% (e.g., about 20%, about 15%, about 10%, about 5%, about 1%, 1-25%, 1-20%, 1-15%, 1-10%, 1-5%, 5-25%, 5-20%, 5-15%, 5-10%, 10-25%, 10-20%, 10- 15%, 15-25%, 15-20%, or 20-25%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by capillary gel electrophoresis-sodium dodecyl sulfate (e.g., as described in Example 9).

In some embodiments, the purity of AAV capsids of the AAV particle is at least 95% (e.g., at least 96%, at least 97%, at least 98%, at least 99%, or 100%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by capillary gel electrophoresis-sodium dodecyl sulfate (e.g., as described in Example 9).

In some embodiments, the purity of AAV capsids of the AAV particle does not differ from a reference value (e.g., baseline value, such as the value at time 0) by more than about 5% (e.g., about 4%, about 3%, about 2%, about 1%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by capillary gel electrophoresis-sodium dodecyl sulfate (e.g., as described in Example 9).

In some embodiments, (a) the pI value (e.g., central pI value) of the AAV capsids of the AAV particle is in the range of 5.8-6.5 (e.g., 5.8-6.4, 5.8-6.3, 5.8-6.2, 5.8-6.1, 5.9-6.4, 5.9-6.3, 5.9-6.2, 5.9-6.1, 6.0-6.4, 6.0-6.3, 6.0-6.2, 6.0-6.1, 6.1-6.4, 6.1-6.3, 6.1-6.2, 6.2-6.4, 6.2-6.3, 6.2-6.2, or 6.3-6.4) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by cIEF (e.g., as described in Example 9), (b) the pI value (e.g., central pI value) of the AAV capsids of the AAV particle does not differ from a reference value (e.g., baseline pI value (e.g., central pI value), such as the pI at time 0) by more than about 10% (e.g., about 9%, about 8%, about 7%, about 6%, about 5%, about 4%, about 3%, about 2%, about 1%, 1-10%, 1-8%, 1-6%, 1-5%, 1-4%, 1-3%, or 1-2%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by cIEF (e.g., as described in Example 9), (c) the pI value (e.g., central pI value) of the AAV capsids of the AAV particle is in the range of 5.9-6.2 (e.g., 5.9-6.0, 5.9-6.1, 6.0-6.1, 6.0-6.2, 6.1-6.2, 5.9, 6.0, 6.1, or 6.2) when the pharmaceutical formulation is stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by cIEF (e.g., as described in Example 9), and/or (d) the pI value (e.g., central pI value) of the AAV capsids of the AAV particle does not differ from a reference value (e.g., baseline pI value (e.g., central pI value), such as the pI at time 0) by more than about 10% (e.g., about 9%, about 8%, about 7%, about 6%, about 5%, about 4%, about 3%, about 2%, about 1%, 1-10%, 1-8%, 1-6%, 1-5%, 1-4%, 1-3%, or 1-2%) when the pharmaceutical formulation is stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by cIEF (e.g., as described in Example 9).

In some embodiments, the AAV viral titer is at least 5×10¹² vg/ml (e.g., at least 6×10¹² vg/ml, at least 7×10¹² vg/ml, at least 8×10¹² vg/ml, at least 9×10¹² vg/ml, at least 1×10¹³ vg/ml, at least 2×10¹³ vg/ml, at least 3×10¹³ vg/ml, at least 4×10¹³ vg/ml, at least 5×10¹³ vg/ml, at least 6×10¹³ vg/ml, at least 7×10¹³ vg/ml, at least 8×10¹³ vg/ml, at least 9×10¹³ vg/ml, at least 1×10¹⁴ vg/ml, 5×10¹² vg/ml to 1×10¹⁴ vg/ml, 5×10¹² vg/ml to 5×10¹³ vg/ml, 1×10¹³ vg/ml to 1×10¹⁴ vg/ml, 1×10¹³ vg/ml to 5×10¹³ vg/ml, or 5×10¹² vg/ml to 5×10¹³ vg/ml) when the pharmaceutical formulation is (a) stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), (b) stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), (c) stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), or (d) subjected to 1-6 freeze thaw cycles (e.g., 1-5, 1-4, 1-3, 1-2, 2-6, 2-5, 2-4, 2-3, 3-6, 3-5, 3-4, 4-6, 4-5, 1, 2, 3, 4, 5, 6 or more freeze thaw cycles), e.g., when assessed by qPCR as described in Example 9.

In some embodiments, (a) the % high molecular weight (% HMW) aggregates in the pharmaceutical formulation is about 10% or less (e.g., 9%, 8%, 7%, 6%, 5%, 4%, 3%, 2%, 1%, 1-10%, 1-9%, 1-8%, 1-7%, 1-6%, 1-5%, or 1-4%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by size exclusion chromatography (e.g., as described in Example 9), (b) the % HMW aggregates in the pharmaceutical formulation does not differ from a reference value (e.g., baseline % HMW, such as % HMW at time 0) by more than about 30% (e.g., about 25%, about 20%, about 15%, about 10%, or about 5%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by size exclusion chromatography (e.g., as described in Example 9), (c) the % HMW aggregates in the pharmaceutical formulation is about 5% or less (e.g., 4%, 3%, 2%, 1%, 1-5%, 1-4%, or 1-3%) when the pharmaceutical formulation is stored at room temperature, e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by size exclusion chromatography (e.g., as described in Example 9), (d) the % HMW aggregates in the pharmaceutical formulation does not differ from a reference value (e.g., baseline % HMW, such as % HMW at time 0) by more than about 30% (e.g., about 25%, about 20%, about 15%, about 10%, or about 5%) when the pharmaceutical formulation is stored at room temperature, e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by size exclusion chromatography (e.g., as described in Example 9), (e) the % HMW aggregates in the pharmaceutical formulation is about 5% or less (e.g., 4%, 3%, 2%, 1%, 1-5%, 1-4%, or 1-3%) when the pharmaceutical formulation is stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by size exclusion chromatography (e.g., as described in Example 9), (f) the % HMW aggregates in the pharmaceutical formulation does not differ from a reference value (e.g., baseline % HMW, such as % HMW at time 0) by more than about 30% (e.g., about 25%, about 20%, about 15%, about 10%, or about 5%) when the pharmaceutical formulation is stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed by size exclusion chromatography (e.g., as described in Example 9), (g) the % HMW aggregates in the pharmaceutical formulation is about 5% or less (e.g., 4%, 3%, 2%, 1%, 1-5%, 1-4%, or 1-3%) when subjected to one or more (e.g., 1-5, 1-4, 1-3, 1-2, 2-6, 2-5, 2-4, 2-3, 3-6, 3-5, 3-4, 4-6, 4-5, 1, 2, 3, 4, 5, 6 or more) freeze thaw cycles (e.g., −80° C. to 25° C. freeze thaw), e.g., when assessed by size exclusion chromatography (e.g., as described in Example 9), and/or (h) the % HMW aggregates in the pharmaceutical formulation does not differ from a reference value (e.g., baseline % HMW, such as % HMW at time 0) by more than about 20% (e.g., about 15%, about 10%, or about 5%) when subjected to one or more (e.g., 1-5, 1-4, 1-3, 1-2, 2-6, 2-5, 2-4, 2-3, 3-6, 3-5, 3-4, 4-6, 4-5, 1, 2, 3, 4, 5, 6 or more) freeze thaw cycles (e.g., −80° C. to 25° C. freeze thaw), e.g., when assessed by size exclusion chromatography (e.g., as described in Example 9).

In some embodiments, (a) the % full capsids of the AAV particle is at least 45% (e.g., at least 50%, at least 60%, at least 70%, 45-70%, 50-70%, 45-60%, or 45-55%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed as described in Example 9, (b) the % full capsids of the AAV particle does not differ from a reference value (e.g., baseline % full capsids at time 0) by more than about 10% (e.g., about 5%, about 3%, about 1%, 1-10%, 1-5%, 1-3%, or 5-10%) when the pharmaceutical formulation is stored at −80° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed as described in Example 9, (c) the % full capsids of the AAV particle is at least 35% (e.g., at least 40%, at least 45%, at least 50%, at least 60%, at least 70%, 35-70%, 35-60%, 35-50%, 35-45%, 40-70%, 40-60% 40-50%, 40-45%, 45-70%, 45-60%, 45-50%, 50-70%, or 50-60%) when the pharmaceutical formulation is stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed as described in Example 9, (d) the % full capsids of the AAV particle does not differ from a reference value (e.g., baseline % full capsids at time 0) by more than about 25% (e.g., about 20%, about 15%, about 10%, or about 5%) when the pharmaceutical formulation is stored at 2-8° C., e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed as described in Example 9, (e) the % full capsids of the AAV particle is at least 30% (e.g., at at least 35%, least 40%, at least 45%, at least 50%, at least 60%, at least 70%, 30-70%, 30-60%, 30-50%, 30-40%, 35-70%, 35-60%, 35-50%, 35-40%, 40-70%, 40-60% 40-50%, 45-70%, 45-60%, 45-50%, 50-70%, or 50-60%) when the pharmaceutical formulation is stored at room temperature, e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed as described in Example 9, and/or (f) the % full capsids of the AAV particle does not differ from a reference value (e.g., baseline % full capsids at time 0) by more than about 40% (e.g., about 35%, about 30%, about 25%, about 20%, about 15%, about 10%, or about 5%) when the pharmaceutical formulation is stored at room temperature, e.g., for 1 day, 2 days, 3 days, 4 days, 5 days, 6 days, 1 week, 2 weeks, 3 weeks, 1 month, 2 months, 3 months, 4 months, 5 months, 6 months, or longer (e.g., one or more years), e.g., when assessed as described in Example 9.

In some embodiments, a formulation described herein is stored at a high concentration and/or a high osmolarity. In some embodiments, the formulation for storage comprises 3-7.5% glycerol. In some embodiments, the formulation is diluted prior to use, e.g., diluted sufficiently to achieve a concentration of glycerol in the formulation of about 5.95%.

In some embodiments, the concentration of the polyether is adjusted to achieve an osmolality (mOsm/kg) within the range of 250-550. In some embodiments, the formulation comprises glycerol (or equivalent) in a concentration that results in an osmolality within the range of 250-550, for example, 250-350, 250-400, 300-400, 350-450, or 450-550 mOsm/kg. In some embodiments, the formulation comprises glycerol at a concentration of 0.75-1.25% or 2.25-2.75%. In some embodiments, the formulation comprises glycerol at a concentration of 1% or about 1%. In some embodiments, the formulation comprises glycerol at a concentration of 2.5% or about 2.5%. In some embodiments, glycerol can be replaced by an amount of glycerin or PEG (e.g., PEG with a molecular weight ≤300) sufficient to achieve an osmolality within the desired range (e.g., between 250-550).

In some embodiments, the concentration of the sugar is adjusted to achieve an osmolality (mOsm/kg) within the range of 250-550. In some embodiments, the formulation comprises trehalose (or equivalent) in a concentration that results in an osmolality within the range of 250-550, for example, 250-350, 250-400, 300-400, 350-450, or 450-550 mOsm/kg. In some embodiments, the formulation comprises trehalose at a concentration of 5.8-6.2%. In some embodiments, the formulation comprises trehalose at a concentration of 5.95% or about 5.95%. In some embodiments, trehalose can be replaced by an amount of a different sugar sufficient to achieve an osmolality within the desired range.

In some embodiments, the formulation comprises (a) an AAV particle or variant thereof described herein, e.g., an AAV particle comprising an AAV capsid variant described herein, (b) Tris, (c) glycerol, (d) sodium chloride, and (e) a poloxamer.

In some embodiments, the formulation comprises (a) an AAV particle or variant thereof described herein, e.g., an AAV particle comprising an AAV capsid variant described herein, (b) 10-30 mM Tris, (c) 0.5-1.5% glycerol, (d) 55-70 mM sodium chloride, and (e) 0.0005-0.0015% Pluronic F68. In some embodiments, the formulation comprises (a) an AAV particle or variant thereof described herein, e.g., an AAV particle comprising an AAV capsid variant described herein, (b) 15-25 mM Tris, (c) 0.8%-1.2% glycerol, (d) 60-65 mM sodium chloride, and (e) 0.0008-0.0012% Pluronic F68. In some embodiments, the formulation comprises (a) an AAV particle or variant thereof described herein, e.g., an AAV particle comprising an AAV capsid variant described herein, (b) about 20 mM Tris, (c) about 1.0% glycerol, (d) about 62.5 mM sodium chloride, and (e) about 0.001% Pluronic F68. In some embodiments, the formulation comprises (a) an AAV particle or variant thereof described herein, e.g., an AAV particle comprising an AAV capsid variant described herein, (b) 20 mM Tris, (c) 1.0% glycerol, (d) 62.5 mM sodium chloride, and (e) 0.001% Pluronic F68. In some embodiments, the formulation has a pH in the range of 7.8-8.5, for example, 8.0-8.3. In some embodiments, the formulation has a pH of 8.0, 8.1, 8.2, 8.3, 8.4, or 8.5, or a pH of about 8.0, 8.1, 8.2, 8.3, 8.4, or 8.5. In some embodiments, the formulation has an osmolality (mOsm/kg) in the range of 250-500 (e.g., 250-400 or 250-350).

In some embodiments, the formulation comprises (a) an AAV particle or variant thereof described herein, e.g., an AAV particle comprising an AAV capsid variant described herein, (b) 10-30 mM Tris, (c) 2.0-3.0% glycerol, (d) 55-70 mM sodium chloride, and (e) 0.0005-0.0015% Pluronic F68. In some embodiments, the formulation comprises (a) an AAV particle or variant thereof described herein, e.g., an AAV particle comprising an AAV capsid variant described herein, (b) 15-25 mM Tris, (c) 2.3%-2.7% glycerol, (d) 60-65 mM sodium chloride, and (e) 0.0008-0.0012% Pluronic F68. In some embodiments, the formulation comprises (a) an AAV particle or variant thereof described herein, e.g., an AAV particle comprising an AAV capsid variant described herein, (b) about 20 mM Tris, (c) about 2.5% glycerol, (d) about 62.5 mM sodium chloride, and (e) about 0.001% Pluronic F68. In some embodiments, the formulation comprises (a) an AAV particle or variant thereof described herein, e.g., an AAV particle comprising an AAV capsid variant described herein, (b) 20 mM Tris, (c) 2.5% glycerol, (d) 62.5 mM sodium chloride, and (e) 0.001% Pluronic F68. In some embodiments, the formulation has a pH in the range of 7.8-8.5, for example, 8.0-8.3. In some embodiments, the formulation has a pH of 8.0, 8.1, 8.2, 8.3, 8.4, or 8.5, or a pH of about 8.0, 8.1, 8.2, 8.3, 8.4, or 8.5. In some embodiments, the formulation has an osmolality (mOsm/kg) in the range of 400-650 (e.g., 450-600 or 450-550).

In some embodiments, the formulation comprises (a) an AAV particle or variant thereof described herein, e.g., an AAV particle comprising an AAV capsid variant described herein, (b) 10-30 mM Tris, (c) 5.5-6.5% trehalose, (d) 55-70 mM sodium chloride, and (e) 0.0005-0.0015% Pluronic F68. In some embodiments, the formulation comprises (a) an AAV particle or variant thereof described herein, e.g., an AAV particle comprising an AAV capsid variant described herein, (b) 15-25 mM Tris, (c) 5.8-6.2% trehalose, (d) 60-65 mM sodium chloride, and (e) 0.0008-0.0012% Pluronic F68. In some embodiments, the formulation comprises (a) an AAV particle or variant thereof described herein, e.g., an AAV particle comprising an AAV capsid variant described herein, (b) about 20 mM Tris, (c) about 5.95% trehalose, (d) about 62.5 mM sodium chloride, and (e) about 0.001% Pluronic F68. In some embodiments, the formulation comprises (a) an AAV particle or variant thereof described herein, e.g., an AAV particle comprising an AAV capsid variant described herein, (b) 20 mM Tris, (c) 5.95% trehalose, (d) 62.5 mM sodium chloride, and (e) 0.001% Pluronic F68. In some embodiments, the formulation has a pH in the range of 7.8-8.5, for example, 8.0-8.3. In some embodiments, the formulation has a pH of 8.0, 8.1, 8.2, 8.3, 8.4, or 8.5, or a pH of about 8.0, 8.1, 8.2, 8.3, 8.4, or 8.5. In some embodiments, the formulation has an osmolality (mOsm/kg) in the range of 250-500 (e.g., 250-400 or 250-350).

In some embodiments, components of the aforementioned formulations are replaced with an equivalent component which does not substantially affect the properties of the formulation (e.g., 1, 2, 3, 4, or all 5 of the aforementioned properties (a)-(e)) under various storage conditions, e.g., as described in Example 9. For example, in some embodiments, Tris is replaced with a different buffering agent, e.g., Bis-tris propane (BTP) or a PBS-based buffering agent (e.g., PBS). In some embodiments, sodium chloride is replaced with a different salt, e.g., potassium chloride. In some embodiments, glycerol is replaced with a different polyether, e.g., glycerin or PEG (e.g., PEG with a molecular weight ≤300). In some embodiments, Pluronic F68 is replaced with another ethylene oxide/propylene oxide copolymer (e.g., Pluronic F127). Whether or not the replaced component has an impact on the properties (e.g., pH, osmolality, AAV titer, occupancy, aggregation) of the formulation can be tested using the assays and methods described herein, e.g., in Example 9.

The formulations described herein comprise an AAV particle or variant thereof described herein, e.g., an AAV particle comprising an AAV capsid variant described herein (e.g., an AAV9 capsid variant). In some embodiments, the formulations comprise an AAV particle comprising the AAV capsid variant of any one of TTD-001, TTD-002, TTD-003, TTD-004, TTD-005, TTD-006, TTD-007, TTD-008, TTD-009, TTD-010, TTD-011, TTD-012, TTD-013, or TTD-014, e.g., as provided in Tables 3 and 4. In some embodiments, the formulation comprises an AAV capsid variant comprising an amino acid sequence in any one of Tables 1A, 1B, 2-7, 10-11, or 20, or a variant thereof. In some embodiments, the formulation comprises an AAV capsid variant comprising the amino acid sequence of any one of SEQ ID NOs: 5, 8, 3636-3647, or a sequence with at least 70% (e.g., at least about 75, 80, 85, 90, 95, 96, 97, 98, or 99%) sequence identity thereto; an amino acid sequence comprising at least one, two or three modifications, but not more than 30, 20 or 10 modifications relative to the amino acid sequence of any one of SEQ ID NOs: 5, 8, 3636-3647; or an amino acid sequence comprising at least one, two or three, but not more than 30, 20 or 10 different amino acids relative to the amino acid sequence of any one of SEQ ID NOs: 5, 8, 3636-3647.

In some embodiments, a formulation described herein comprises an AAV particle comprising an AAV capsid variant comprising the amino acid sequence of SEQ ID NO: 3636, or a sequence with at least 70% (e.g., at least about 75, 80, 85, 90, 95, 96, 97, 98, or 99%) sequence identity thereto; an amino acid sequence comprising at least one, two or three modifications, but not more than 30, 20 or 10 modifications relative to the amino acid sequence of SEQ ID NO: 3636; or an amino acid sequence comprising at least one, two or three, but not more than 30, 20 or 10 different amino acids relative to the amino acid sequence of SEQ ID NOs: 3636.

In some embodiments, a formulation described herein comprises an AAV particle comprising an AAV capsid variant comprising the amino acid sequence of SEQ ID NO: 5, or a sequence with at least 70% (e.g., at least about 75, 80, 85, 90, 95, 96, 97, 98, or 99%) sequence identity thereto; an amino acid sequence comprising at least one, two or three modifications, but not more than 30, 20 or 10 modifications relative to the amino acid sequence of SEQ ID NO: 5; or an amino acid sequence comprising at least one, two or three, but not more than 30, 20 or 10 different amino acids relative to the amino acid sequence of SEQ ID NOs: 5.

In some embodiments, a formulation described herein comprises an AAV particle comprising an AAV capsid variant comprising the amino acid sequence of SEQ ID NO: 8, or a sequence with at least 70% (e.g., at least about 75, 80, 85, 90, 95, 96, 97, 98, or 99%) sequence identity thereto; an amino acid sequence comprising at least one, two or three modifications, but not more than 30, 20 or 10 modifications relative to the amino acid sequence of SEQ ID NO: 8; or an amino acid sequence comprising at least one, two or three, but not more than 30, 20 or 10 different amino acids relative to the amino acid sequence of SEQ ID NOs: 8.

In some embodiments, a formulation described herein, e.g., comprising an AAV particle or variant thereof comprising an AAV capsid variant described herein, is administered to a subject (e.g., a human) intravenously. In some embodiments, the formulation to be administered intravenously is an isotonic solution (e.g., a solution comprising an osmolarity of 270-310 mOsm/L). In some embodiments, the formulation to be administered intravenously is a solution comprising an osmolarity above 310 mOsm/L. In some embodiments, the formulation is administered in a high volume (e.g., a volume greater than 100 mL). In some embodiments, the formulation to be administered (e.g., intravenously) in a high volume (e.g., a volume greater than 100 mL) comprises a concentration of about 0.5-3% (e.g., about 1-2%) glycerol and/or an osmolarity of about 270-310 mOsm/L. In some embodiments, a high volume comprises a volume above 100 mL. In some embodiments, the formulation is administered in a low volume (e.g., a volume of 100 mL or a volume lower than 100 mL). In some embodiments, the formulation to be administered (e.g., intravenously) in a low volume (e.g., a volume of 100 mL or a volume lower than 100 mL) comprises a concentration of about 3-5% glycerol and/or an osmolarity above 310 mOsm/L. In some embodiments a low volume comprises 100 mL. In some embodiments, a low volume comprises no more than 100 mL.

### Administration

In some embodiments, an AAV particle disclosed herein (e.g., an AAV particle comprising an AAV capsid variant) may be administered by a to a subject by a delivery route, e.g., a localized delivery route or a systemic delivery route.

In some embodiments, an AAV particle described herein (e.g., an AAV particle comprising an AAV capsid variant) may be administered via such a route that it is able to cross the blood-brain barrier, vascular barrier, or other epithelial barrier. In some embodiments, an AAV particle of the present disclosure (e.g., an AAV particle comprising an AAV capsid variant) may be administered in any suitable form, either as a liquid solution or suspension, as a solid form suitable for liquid solution or suspension in a liquid solution. In some embodiments, an AAV particle (e.g., an AAV particle comprising an AAV capsid variant) may be formulated with any appropriate and pharmaceutically acceptable excipient.

In some embodiments, the AAV particle described herein (e.g., an AAV particle comprising an AAV capsid variant) is administered intramuscularly, intravenously, intracerebrally, intrathecally, intracerebroventricularly, via intraparenchymal administration, or via intra-cisterna magna injection (ICM).

In some embodiments, an AAV particle of the present disclosure (e.g., an AAV particle comprising an AAV capsid variant) may be delivered to a subject via a single route administration. In some embodiments, an AAV particle of the present disclosure may be delivered to a subject via a multi-site route of administration. In some embodiments, a subject may be administered at 2, 3, 4, 5, or more than 5 sites.

In some embodiments, an AAV particle of the present disclosure (e.g., an AAV particle comprising an AAV capsid variant) is administered via a bolus infusion. In some embodiments, an AAV particle of the present disclosure is administered via sustained delivery over a period of minutes, hours, or days. In some embodiments, the infusion rate may be changed depending on the subject, distribution, formulation, and/or another delivery parameter. In some embodiments, an AAV particle of the present disclosure is administered using a controlled release. In some embodiments, an AAV particle of the present disclosure is administered using a sustained release, e.g., a release profile that conforms to a release rate over a specific period of time.

In some embodiments, an AAV particle (e.g., an AAV particle comprising an AAV capsid variant) may be delivered by more than one route of administration. As non-limiting examples of combination administrations, an AAV particle may be delivered by intrathecal and intracerebroventricular, or by intravenous and intraparenchymal administration.

### Intravenous Administration

In some embodiments, an AAV particle described herein (e.g., an AAV particle comprising an AAV capsid polypeptide, e.g., an AAV capsid variant) may be administered to a subject by systemic administration. In some embodiments, the systemic administration is intravenous administration. In another embodiment, the systemic administration is intraarterial administration. In some embodiments, an AAV particle of the present disclosure may be administered to a subject by intravenous administration. In some embodiments, the intravenous administration may be achieved by subcutaneous delivery. In some embodiments, the AAV particle is administered to the subject via focused ultrasound (FUS), e.g., coupled with the intravenous administration of microbubbles (FUS-MB) or MRI-guided FUS coupled with intravenous administration, e.g., as described in Terstappen et al. (Nat Rev Drug Discovery, doi.org/10.1038/s41573-021-00139-y (2021)), the contents of which are incorporated herein by reference in its entirety. In some embodiments, the AAV particle is administered to the subject intravenously. In some embodiments, the subject is a human.

### Administration to the CNS

In some embodiments, an AAV particle described herein (e.g., an AAV particle comprising an AAV capsid variant) may be delivered by direct injection into the brain. As a non-limiting example, the brain delivery may be by intrahippocampal administration. In some embodiments, an AAV particle of the present disclosure may be administered to a subject by intraparenchymal administration. In some embodiments, the intraparenchymal administration is to tissue of the central nervous system. In some embodiments, an AAV particle of the present disclosure may be administered to a subject by intracranial delivery (See, e.g., U.S. Pat. No. 8,119,611; the content of which is incorporated herein by reference in its entirety). In some embodiments, an AAV particle described herein may be delivered by injection into the CSF pathway. Non-limiting examples of delivery to the CSF pathway include intrathecal and intracerebroventricular administration. In some embodiments, an AAV particle described herein may be administered via intracisternal magna (ICM) injection.

In some embodiments, an AAV particle of the present disclosure (e.g., an AAV particle comprising an AAV capsid variant) may be delivered to the brain by systemic delivery. As a non-limiting example, the systemic delivery may be by intravascular administration. As a non-limiting example, the systemic or intravascular administration may be intravenous.

In some embodiments, an AAV particle (e.g., an AAV particle comprising an AAV capsid variant) of the present disclosure may be delivered by an intraocular delivery route. A non-limiting example of an intraocular administration includes an intravitreal injection.

### Intramuscular Administration

In some embodiments, an AAV particle described herein (e.g., an AAV particle comprising an AAV capsid variant) may be delivered by intramuscular administration. Without wishing to be bound by theory, it is believed in some embodiments, that the multi-nucleated nature of muscle cells provides an advantage to gene transduction subsequent to AAV delivery. In some embodiments, cells of the muscle are capable of expressing recombinant proteins with the appropriate post-translational modifications. Without wishing to be bound by theory, it is believed in some embodiments, the enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered. In some embodiments, an AAV particle of the present disclosure may be delivered by an intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, an AAV particle of the present disclosure (e.g., an AAV particle comprising an AAV capsid variant) is administered to a subject and transduces the muscle of a subject. As a non-limiting example, an AAV particle is administered by intramuscular administration. In some embodiments, an AAV particle of the present disclosure may be administered to a subject by subcutaneous administration. In some embodiments, the intramuscular administration is via systemic delivery. In some embodiments, the intramuscular administration is via intravenous delivery. In some embodiments, the intramuscular administration is via direct injection to the muscle.

In some embodiments, the muscle is transduced by administration, e.g., intramuscular administration. In some embodiments, an intramuscular delivery comprises administration at one site. In some embodiments, an intramuscular delivery comprises administration at more than one site. In some embodiments, an intramuscular delivery comprises administration at two, three, four, or more sites. In some embodiments, intramuscular delivery is combined with at least one other method of administration.

In some embodiments, an AAV particle pf the present disclosure (e.g., an AAV particle comprising an AAV capsid variant) may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U. S. Patent Publication Nos. US20100240739 and US20100130594; the content of each of which is incorporated herein by reference in their entirety).

In some embodiments, an AAV particle of the present disclosure (e.g., an AAV particle comprising an AAV capsid variant) may be administered to a subject by intraparenchymal administration. In some embodiments, the intraparenchymal administration is to muscle tissue. In some embodiments, an AAV particle of the present disclosure is delivered as described in Bright et al 2015 (Neurobiol Aging. 36(2):693-709), the contents of which are herein incorporated by reference in their entirety. In some embodiments, an AAV particle of the present disclosure is administered to the gastrocnemius muscle of a subject. In some embodiments, an AAV particle of the present disclosure is administered to the bicep femorii of the subject. In some embodiments, an AAV particles of the present disclosure is administered to the tibialis anterior muscles. In some embodiments, an AAV particle of the present disclosure is administered to the soleus muscle.

### Depot Administration

As described herein, in some embodiments, a pharmaceutical composition and/or an AAV particle of the present disclosure (e.g., an AAV particle comprising an AAV capsid polypeptide, e.g., an AAV capsid variant) are formulated in depots for extended release. Generally, specific organs or tissues are targeted for administration.

In some embodiments, a pharmaceutical composition and/or an AAV particle of the present disclosure (e.g., an AAV particle comprising an AAV capsid polypeptide, e.g., an AAV capsid variant) are spatially retained within or proximal to target tissues. Provided are methods of providing a pharmaceutical composition, an AAV particle, to target tissues of mammalian subjects by contacting target tissues (which comprise one or more target cells) with the pharmaceutical composition and/or the AAV particle, under conditions such that they are substantially retained in target tissues, e.g., such that at least 10, 20, 30, 40, 50, 60, 70, 80, 85, 90, 95, 96, 97, 98, 99, 99.9, 99.99 or greater than 99.99% of the composition is retained in the target tissues. In some embodiments, retention is determined by measuring the amount of pharmaceutical composition and/or AAV particle, that enter a target cell or a plurality of target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of a pharmaceutical composition and/or an AAV particle, administered to a subject are present intracellularly at a period of time following administration. For example, intramuscular injection to a subject may be performed using aqueous compositions comprising a pharmaceutical composition and/or an AAV particle of the present disclosure and a transfection reagent, and retention is determined by measuring the amount of the pharmaceutical composition and/or the AAV particle, present in the muscle cell or plurality of muscle cells.

In some embodiments, disclosed herein are methods of providing a pharmaceutical composition and/or an AAV particle of the present disclosure (e.g., an AAV particle comprising an AAV capsid polypeptide, e.g., an AAV capsid variant) to a tissue of a subject, by contacting the tissue (comprising a cell, e.g., a plurality of cells) with the pharmaceutical composition and/or the AAV particle under conditions such that they are substantially retained in the tissue. In some embodiments, a pharmaceutical composition and/or AAV particle described herein comprise a sufficient amount of an active ingredient such that the effect of interest is produced in at least one cell. In some embodiments, a pharmaceutical composition and/or an AAV particle generally comprise one or more cell penetration agents. In some embodiments, the disclosure provides a naked formulations (such as without cell penetration agents or other agents), with or without pharmaceutically acceptable carriers.

### Methods of Treatment

Provided in the present disclosure are methods for introducing (e.g., delivering) an AAV particle of the present disclosure (e.g., an AAV particle comprising an AAV capsid variant) into cells. In some embodiments, the method comprises introducing into said cells an AAV particle or vector described herein in an amount sufficient to modulate, e.g., increase, the production of a target gene, mRNA, and/or protein. In some embodiments, the method comprises introducing into said cells an AAV particle or vector described herein in an amount sufficient to modulate, e.g., decrease, expression of a target gene, mRNA, and/or protein. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes. In other aspects, the cells may be a muscle cell, e.g., a cell of a diaphragm, a quadriceps, or a heart (e.g., a heart atrium or a heart ventricle).

Disclosed in the present disclosure are methods for treating a neurological disease/disorder or a neurodegenerative disorder, a muscular or neuromuscular disorder, or a neurooncological disorder associated with aberrant, e.g., insufficient or increased, function/presence of a protein, e.g., a target protein in a subject in need of treatment.

In some embodiments, the method comprises administering to the subject a therapeutically effective amount of a composition comprising AAV particles of the present disclosure. As a non-limiting example, the AAV particles can increase target gene expression, increase target protein production, and thus reduce one or more symptoms of neurological disease in the subject such that the subject is therapeutically treated.

In other embodiments, the method comprises administering to the subject a therapeutically effective amount of a composition comprising AAV particles (e.g., an AAV particle comprising an AAV capsid polypeptide, e.g., an AAV capsid variant) comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles of the present disclosure (e.g., an AAV particle comprising an AAV capsid variant described herein) is administered to the central nervous system of the subject via systemic administration. In some embodiments, the systemic administration is intravenous (IV) injection. In some embodiments, the AAV particle described herein or a pharmaceutical composition comprising an AAV particle described herein is administered by focused ultrasound (FUS), e.g., coupled with the intravenous administration of microbubbles (FUS-MB) or MRI-guided FUS coupled with intravenous administration. In some embodiments, a composition comprising an AAV particle described herein is administered intravenously. In some embodiments, the AAV particle is administered at a dose of about 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg. In some embodiments, the AAV particle is administered at a dose of about 6.7e10 VG/kg to 6.7e12 VG/kg, about 1.3e11 VG/kg to 3.4e12 VG/kg, or about 2.2e11 VG/kg to 2e12 VG/kg. In some embodiments, the AAV particle is administered at a dose of about 4e11 VG/kg to 8e11 VG/kg (e.g., about 6.7e11 VG/kg). In some embodiments, the AAV particle is administered at a dose of about 6.7e11 VG/kg. In some embodiments, the AAV particle is administered at a dose of about 2e11 VG/kg to 2e13 VG/kg, about 4e11 VG/kg to 1e13 VG/kg, about 6.7e11 VG/kg to about 6e12 VG/kg. In some embodiments, the AAV particle is administered at a dose of about 1e12 VG/kg to 5e12 VG/kg (e.g., about 2e12 VG/kg). In some embodiments, the AAV particle is administered at a dose of about 2e12 VG/kg. In some embodiments, the AAV particle is administered at a dose of about 6.7e11 VG/kg to 6.7e13 VG/kg, about 1.3e12 VG/kg to 3.4e13 VG/kg, or about 2.2e12 VG/kg to 2e13 VG/kg. In some embodiments, the AAV particle is administered at a dose of about 4e12 VG/kg to 8e12 VG/kg (e.g., 6.7e12 VG/kg). In some embodiments, the AAV particle is administered at a dose of about 6.7e12 VG/kg. In some embodiments, the AAV particle is administered at a dose of about 2e12 VG/kg to 2e14 VG/kg, about 4e12 VG/kg to 1e14 VG/kg, about 6.7e12 VG/kg to about 6e13 VG/kg. In some embodiments, the AAV particle is administered at a dose of about 1e13 VG/kg to 5e13 VG/kg (e.g., 2e13 VG/kg). In some embodiments, the AAV particle is administered at a dose of about 2e13 VG/kg. In some embodiments, the AAV particle comprises a viral genome which is single stranded.

In some embodiments, administration of the AAV particle at a dose of 2e13 VG/kg is capable of transducing greater than 40% of total cells in brain region chosen from a thalamus, caudate, or putamen, and greater than 20% total cells in a brain region chosen from a entorhinal cortex, auditory cortex, or hippocampus. In some embodiments, administration of the AAV particle at a dose of 6.7e12 VG/kg is capable of transducing greater than 20% of total cells in brain region chosen from a thalamus, caudate, putamen, or cerebellum. In some embodiments, administration of the AAV particle at a dose of 2e13 VG/kg is capable of transducing greater than 90% SMI311-positive neurons in the thalamus, dentate and spinal cord. In some embodiments, administration of the AAV particle at a dose of 2e12 VG/kg is capable of expressing transgene mRNA at a supraphysiological level. In some embodiments, administration of the AAV particle at a dose of 2e12 VG/kg is capable of transducing multiple regions of the central nervous system (e.g., one or more regions of the brain and/or spinal cord).

In some embodiments, the composition comprising the AAV particle of the present disclosure (e.g., an AAV particle comprising an AAV capsid variant) is administered to the central nervous system of the subject via intraventricular administration. In some embodiments, the composition comprising the AAV particle of the present disclosure (e.g., an AAV particle comprising an AAV capsid variant) is administered via intra-cisterna magna injection (ICM).

In some embodiments, the composition comprising an AAV particle of the present disclosure (e.g., an AAV particle comprising an AAV capsid variant) is administered to the central nervous system of the subject via intraventricular injection and intravenous injection.

In some embodiments, the composition comprising the AAV particle of the present disclosure (e.g., an AAV particle comprising an AAV capsid variant) is administered to the central nervous system of the subject via ICM injection and intravenous injection at a specific dose per subject. As a non-limiting example, the AAV particles are administered via ICM injection at a dose of 1×10⁴ VG per subject. As a non-limiting example, the AAV particles are administered via IV injection at a dose of 2×10¹³ VG per subject.

In some embodiments, the composition comprising the AAV particle of the present disclosure (e.g., an AAV particle comprising an AAV capsid variant) is administered to the central nervous system of the subject. In other embodiments, the composition comprising the AAV particles of the present disclosure is administered to a CNS tissue of a subject (e.g., putamen, hippocampus, thalamus, or cortex of the subject).

In some embodiments, the composition comprising the AAV particle of the present disclosure (e.g., an AAV particle comprising an AAV capsid variant) is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In some embodiments, the composition comprising the AAV particle of the present disclosure (e.g., an AAV particle comprising an AAV capsid variant) is administered to the central nervous system of the subject via intraparenchymal injection and intravenous injection.

In some embodiments, the composition comprising the AAV particle of the present disclosure (e.g., an AAV particle comprising an AAV capsid variant) is administered to the central nervous system of the subject via intraventricular injection, intraparenchymal injection and intravenous injection.

In some embodiments, the composition comprising an AAV particle (e.g., an AAV particle comprising an AAV capsid variant) of a plurality of particles of the present disclosure is administered to a muscle of the subject via intravenous injection. In some embodiments, the composition comprising an AAV particle of a plurality of particles of the present disclosure is administered to a muscle of the subject via intramuscular injection.

In some embodiments, an AAV particle of the present disclosure (e.g., an AAV particle comprising an AAV capsid variant) may be delivered into specific types of cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells. In some embodiments, an AAV particle of the present disclosure may be delivered into a muscle cell, e.g., a cell of the quadriceps, diaphragm, liver, and/or heart (e.g., heart atrium or heart ventricle).

In some embodiments, an AAV particle (e.g., an AAV particle comprising an AAV capsid polypeptide, e.g., an AAV capsid variant), e.g., a plurality of particles, of the present disclosure may be delivered to a cell or region of the midbrain. In some embodiments, an AAV particle (e.g., an AAV particle comprising an AAV capsid polypeptide, e.g., an AAV capsid variant), e.g., a plurality of particles, of the present disclosure may be delivered to a cell or region of the brains stem.

In some embodiments, an AAV particle (e.g., an AAV particle comprising an AAV capsid polypeptide, e.g., an AAV capsid variant), e.g., a plurality of particles, of the present disclosure may be delivered to neurons in the putamen, hippocampus, thalamus and/or cortex.

In some embodiments, an AAV particle (e.g., an AAV particle comprising an AAV capsid variant), e.g., a plurality of particles, of the present disclosure may be used as a therapy for a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein (e.g., an AAV particle comprising an AAV capsid variant described herein) for a gene replacement therapy.

In some embodiments, an AAV particle (e.g., an AAV particle comprising an AAV capsid variant), e.g., a plurality of particles, of the present disclosure may be used as a therapy for a neurological disease.

In some embodiments, an AAV particle (e.g., an AAV particle comprising an AAV capsid variant), e.g., a plurality of particles, of the present disclosure may be used as a therapy for tauopathies.

In some embodiments, an AAV particle (e.g., an AAV particle comprising an AAV capsid variant), e.g., a plurality of particles, of the present disclosure may be used as a therapy for Alzheimer's Disease.

In some embodiments, an AAV particle (e.g., an AAV particle comprising an AAV capsid variant), e.g., a plurality of particles, of the present disclosure may be used as a therapy for Amyotrophic Lateral Sclerosis.

In some embodiments, an AAV particle (e.g., an AAV particle comprising an AAV capsid variant), e.g., a plurality of particles, of the present disclosure may be used as a therapy for Huntington's Disease.

In some embodiments, an AAV particle (e.g., an AAV particle comprising an AAV capsid variant), e.g., a plurality of particles, of the present disclosure may be used as a therapy for Parkinson's Disease.

In some embodiments, an AAV particle (e.g., an AAV particle comprising an AAV capsid variant), e.g., a plurality of particles, of the present disclosure may be used as a therapy for Gaucher disease (GD) (e.g., Type 1 GD, Type 2 GD, or Type 3 GD). In some embodiments, an AAV particle (e.g., an AAV particle comprising an AAV capsid variant), e.g., a plurality of particles, of the present disclosure may be used as a therapy for Parkinson's disease associated with a GBA mutation. In some embodiments, an AAV particle (e.g., an AAV particle comprising an AAV capsid variant), e.g., a plurality of particles, of the present disclosure may be used as a therapy for dementia with Lewy Bodies (DLB).

In some embodiments, an AAV particle (e.g., an AAV particle comprising an AAV capsid variant), e.g., a plurality of particles, of the present disclosure may be used as a therapy for spinal muscular atrophy.

In some embodiments, an AAV particle (e.g., an AAV particle comprising an AAV capsid variant), e.g., a plurality of particles, of the present disclosure may be used as a therapy for a leukodystrophy, e.g., Alexander disease, autosomal dominant leukodystrophy with autonomic diseases (ADLD), Canavan disease, cerebrotendinous xanthomatosis (CTX), metachromatic leukodystrophy (MLD), Pelizaeus-Merzbacher disease, or Refsum disease.

In some embodiments, an AAV particle (e.g., an AAV particle comprising an AAV capsid variant), e.g., a plurality of particles, of the present disclosure may be used as a therapy for Friedreich's Ataxia.

In some embodiments, an AAV particle (e.g., an AAV particle comprising an AAV capsid variant), e.g., a plurality of particles, of the present disclosure may be used as a therapy for chronic or neuropathic pain.

In some embodiments, an AAV particle (e.g., an AAV particle comprising an AAV capsid variant), e.g., a plurality of particles, of the present disclosure may be used as a therapy for a disease associated with expression of HER2, e.g., a disease associated with overexpression of HER2. In some embodiments, the AAV particle of the disclosure (e.g., an AAV particle comprising an AAV capsid variant) is useful for the treatment, prophylaxis, palliation or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, an AAV particle (e.g., an AAV particle comprising an AAV capsid variant) e.g., a plurality of particles, of the present disclosure may be used as a therapy for a neuro-oncological disorder. In some embodiments, the neuro-oncological disorder is a cancer of primary CNS origin (e.g., a cancer of a CNS cell and/or CNS tissue). In some embodiments, the neuro-oncological disorder is metastatic cancer in a CNS cell, CNS region, and/or a CNS tissue. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, an AAV particle (e.g., an AAV particle comprising an AAV capsid variant), e.g., a plurality of particles, of the present disclosure may be used as a therapy for a muscular disorder or a neuromuscular disorder.

In some embodiments, an AAV particle (e.g., an AAV particle comprising an AAV capsid variant), e.g., a plurality of particles, of the present disclosure may be used as a therapy for a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction.

In some embodiments, administration of the AAV particle described herein (e.g., an AAV particle comprising an AAV capsid polypeptide, e.g., an AAV capsid variant) to a subject may increase target gene, mRNA, and/or protein levels in a subject, relative to a control, e.g., the gene, mRNA, and/or mRNA levels in the subject prior to receiving AAV particle. The target gene, mRNA, and/or protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may increase the gene, mRNA, and/or protein levels of a target protein by fold increases over baseline. In some embodiments, AAV particles lead to 5-6 times higher levels of a target gene, mRNA, or protein.

In some embodiments, administration of the AAV particle described herein (e.g., an AAV particle comprising an AAV capsid polypeptide, e.g., an AAV capsid variant), e.g., an AAV particle comprising a nucleic acid encoding a siRNA molecule or an antibody or antibody fragment, to a subject may decrease target gene, mRNA, and/or protein levels in a subject, relative to a control, e.g., the gene, mRNA, and/or mRNA levels in the subject prior to receiving AAV particle. The target gene, mRNA, and/or protein levels may be decreased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may decrease the gene, mRNA, and/or protein levels of a target protein by fold decreases over baseline.

In some embodiments, the AAV particles of the present disclosure (e.g., an AAV particle comprising an AAV capsid polypeptide, e.g., an AAV capsid variant) may be used to increase target protein and reduce symptoms of neurological disease in a subject. In some embodiments, the AAV particles of the present disclosure (e.g., an AAV particle comprising an AAV capsid polypeptide, e.g., an AAV capsid variant) may be used to decrease target protein and reduce symptoms of neurological disease in a subject.

In some embodiments, the AAV particles of the present disclosure (e.g., an AAV particle comprising an AAV capsid polypeptide, e.g., an AAV capsid variant) may be used to reduce the decline of functional capacity and activities of daily living as measured by a standard evaluation system such as, but not limited to, the total functional capacity (TFC) scale.

In some embodiments, the AAV particles of the present disclosure (e.g., an AAV particle comprising an AAV capsid polypeptide, e.g., an AAV capsid variant) may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

In some embodiments, the present composition is administered as a solo therapeutic or as combination therapeutic for the treatment of a neurological disease/disorder or a neurodegenerative disorder, a muscular disorder or neuromuscular disorder, and/or a neuro-oncological disorder.

The AAV particles (e.g., an AAV particle comprising an AAV capsid variant) encoding the target protein may be used in combination with one or more other therapeutic agents. In some embodiments, compositions can be administered concurrently with, prior to, or subsequent to, additional therapeutic or medical procedures. In general, each agent will be administered at a dose and/or on a time schedule determined for that agent.

Therapeutic agents that may be used in combination with the AAV particles of the present disclosure (e.g., an AAV particle comprising an AAV capsid variant) can be small molecule compounds which are antioxidants, anti-inflammatory agents, anti-apoptosis agents, calcium regulators, anti-glutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles of the present disclosure (e.g., an AAV particle comprising an AAV capsid variant) for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-1, GDNF, BDNF, CTNF, VEGF, Colivelin, Xaliproden, Thyrotrophin-releasing hormone and ADNF, and variants thereof.

In one aspect, the AAV particle described herein (e.g., an AAV particle comprising an AAV capsid variant) may be co-administered with AAV particles expressing neurotrophic factors such as AAV-IGF-1 (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In some embodiments, administration of the AAV particles (e.g., an AAV particle comprising an AAV capsid variant) to a subject will modulate, e.g., increase or decrease, the expression of a target protein in a subject and the modulation, e.g., increase or decrease of the presence, level, activity, and/or expression of the target protein will reduce the effects and/or symptoms of a neurological disease/disorder or a neurodegenerative disorder, a muscular disorder or neuromuscular disorder, and/or a neuro-oncological disorder in a subject.

### Definitions

Unless defined otherwise, all technical and scientific terms used herein have the same meaning as commonly understood by one of ordinary skill in the art to which the invention pertains.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” and “consisting essentially thereof” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Amplicon: As used herein, “amplicon” may refer to any piece of RNA or DNA formed as the product of amplification events, e.g. PCR. In some embodiments, full-length capsid amplicons may be used as templates for next generation sequencing (NGS) library generation. Full-length capsid amplicons may be used for cloning into a DNA library for any number of additional rounds of AAV selection as described herein.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of a gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biopanning: As used herein, the term “biopanning” refers to an AAV capsid library selection process comprising administration of an AAV particle with enhanced tissue- and/or cell type-specific transduction to a cell and/or subject; extraction of nucleotides encoded by said AAV particle from said transduced tissue- and/or cell type-specific; and, use of the extracted nucleotides for cloning into a nucleotide library for the generation of AAV particles for subsequent rounds of the same.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. The term “complementary” as used herein can encompass fully complementary, partially complementary, or substantially complementary. As used herein, the term “substantially complementary” means that the siRNA has a sequence (e.g., in the antisense strand) which is sufficient to bind the desired target mRNA, and to trigger the RNA silencing of the target mRNA. “Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Element: As used herein, the term “element” refers to a distinct portion of an entity. In some embodiments, an element may be a polynucleotide sequence with a specific purpose, incorporated into a longer polynucleotide sequence.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95%, 90%, 85%, 80%, 70%, 60% or less. For example, gaps or discontinuities may be present in the capsid so long as the viral genome is retained in the capsid, e.g., prior to entry into a cell.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Formulation: As used herein, a “formulation” includes at least one AAV particle (active ingredient) and an excipient, and/or an inactive ingredient.

Fragment: A “fragment,” as used herein, refers to a portion. For example, an antibody fragment may comprise a CDR, or a heavy chain variable region, or a scFv, etc.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Inverted terminal repeat: As used herein, the term “inverted terminal repeat” or “ITR” refers to a cis-regulatory element for the packaging of polynucleotide sequences into viral capsids.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Library: As used herein, the term “library” refers to a diverse collection of linear polypeptides, polynucleotides, viral particles, or viral vectors. As examples, a library may be a DNA library or an AAV capsid library.

Molecular scaffold: As used herein a “molecular scaffold” is a framework or starting molecule that forms the sequence or structural basis against which to design or make a subsequent molecule.

Neurological disease: As used herein, a “neurological disease” is any disease associated with the central or peripheral nervous system and components thereof (e.g., neurons).

Orthogonal evolution: As used herein, the term “orthogonal evolution” refers to a method wherein AAV particles are administered for a first round of AAV selection as described herein across a set of any number of cell- and/or subject-types that may be from different species and/or strains, and wherein any number of additional, i.e., subsequent, AAV selection rounds are performed either across a set of any number of cell- and/or subject-types that may be from different species and/or strains, or across a set of any number of cell- and/or subject-types that may be from the same species and/or strain.

Open reading frame: As used herein, “open reading frame” or “ORF” refers to a sequence which does not contain a stop codon in a given reading frame.

Particle: As used herein, a “particle” is a virus comprised of at least two components, a protein capsid and a polynucleotide sequence enclosed within the capsid.

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

RNAi agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

miR binding site: As used herein, a “miR binding site” comprises a nucleic acid sequence (whether RNA or DNA, e.g., differ by “U” of RNA or “T” in DNA) that is capable of binding, or binds, in whole or in part to a microRNA (miR) through complete or partial hybridization. Typically, such binding occurs between the miR and the miR binding site in the reverse complement orientation. In some embodiments, the miR binding site is transcribed from the AAV vector genome encoding the miR binding site.

In some embodiments, a miR binding site may be encoded or transcribed in series. Such a “miR binding site series” or “miR BSs” may include two or more miR binding sites having the same or different nucleic acid sequence.

Spacer: As used here, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

Sample: As used herein, the term “sample” or “biological sample” refers to a subset of its tissues, cells, nucleic acids, or component parts (e.g. body fluids, including but not limited to blood, serum, mucus, lymphatic fluid, synovial fluid, cerebrospinal fluid, saliva, amniotic fluid, amniotic cord blood, urine, vaginal fluid and semen).

Self-complementary viral particle: As used herein, a “self-complementary viral particle” is a particle comprised of at least two components, a protein capsid and a self-complementary viral genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target Cells: As used herein, “target cells” or “target tissue” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Variant: As used herein, the term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: As used herein, the term “functional variant” refers to a polypeptide variant or a polynucleotide variant that has at least one activity of the reference sequence.

Insertional Variant: “Insertional variants” when referring to polypeptides are those with one or more amino acids inserted, e.g., immediately adjacent or subsequent, to a position in an amino acid sequence. “Immediately adjacent” or “immediately subsequent” to an amino acid means connected to either the alpha-carboxy or alpha-amino functional group of the amino acid.

Deletional Variant: “Deletional variants” when referring to polypeptides, are those with one or more amino acids in deleted from a reference protein.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral Genome: As used herein, the terms “viral genome” or “vector genome” refer to the nucleic acid sequence(s) encapsulated in an AAV particle. A viral genome comprises a nucleic acid sequence with at least one payload region encoding a payload and at least one ITR.

### EQUIVALENTS AND SCOPE

The disclosures of each and every patent, patent application, and publication cited herein are hereby incorporated herein by reference in their entirety. Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1. Peptide Display Capsid Library Configuration

Peptide display capsid libraries are configured by insertion of randomized n-mer amino acids such as, but not limited to, 5-mer, 6-mer, 7-mer and/or 9-mer amino acids, into the surface-exposed hypervariable loop I, loop IV, loop VI, and/or loop VIII region of any AAV capsid serotype, including AAV5, AAV6, or AAV-DJ8, as well as AAV9 capsids, and/or variants thereof. The genes encoding the peptide display capsid library are under the control of any promotor, depending on the desired tropism, e.g., a neuron-specific synapsin promoter (SYN or Syn), or an astrocyte-specific GFAP promoter.

Peptide display capsid libraries are further configured such that the n-mer peptide insertion(s) follows a contiguous (or continuous) design and/or a noncontiguous (or noncontinuous), or split design, or combination thereof, with insertion position(s) mapped using a sliding window algorithm. As a non-limiting example, the peptide insertion may be an AAV9 6-mer contiguous peptide insertion with a sliding window originating at any amino acid position, e.g., amino acids 454-461. As another non-limiting example, the peptide insertion may be an AAV9 3-mer peptide split design or contiguous peptide insertion with a sliding window originating at any amino acid position, e.g., amino acids 586-588. As yet another non-limiting example, the peptide insertion may be an AAV9 6-mer and/or 7-mer peptide contiguous peptide insertion with a sliding window originating at any amino acid position, e.g., amino acids 585-590.

Any number of such configured peptide display capsid libraries may be pooled in a cell and/or subject, including a non-human primate (NHP) cell and/or subject, and administered to any tissue (e.g., central nervous system tissue) via any route, including but not limited to IV and/or ICM injection, at any VG/cell and/or VG/subject dose. As a non-limiting example, six configured peptide display capsid libraries are pooled and administered to the central nervous system of an NHP via intravenous administration of dose 1×10¹⁴ VG/NHP. As another non-limiting example, six libraries are pooled and administered to the central nervous system of NHP via an intraventricular administration, such as, but not limited intraventricular administration that is an intra-cisterna magna injection (ICM) of dose 2×10¹³ a VG/NHP.

### Example 2. Identification and Design of Non-Human Primate AAV Capsid Libraries

A TRACER RNA-driven library selection for increased nervous system tissue transduction in a non-human primate (NHP) is developed and carried out in accordance with methods similar, or equivalent, to those described in WO2020072683, the contents of which are herein incorporated by reference in their entirety, particularly as pertains to the TRACER method.

AAV libraries, e.g., AAV9 libraries, generated are administered by any route to NHPs at a given VG dose(s) per animal. A number of groups of NHPs are administered promoter-driven (e.g., SYN-driven or GFAP-driven) libraries derived from wild-type AAV9 flanking sequences, while other groups receive pooled libraries containing wild-type, PHP.eB-derived, or other AAV serotype sequences. After a period, RNA is extracted from a tissue, such as but not limited to spinal cord and brain tissue. The RNA preparation is subjected to mRNA enrichment. The enriched mRNA is reverse transcribed to cDNA. The cDNA is amplified. This method allows recovery of abundant amplicons from tissue samples.

Full-length capsid amplicons are used as templates for NGS library generation, as well as cloning into a DNA library for the next, or subsequent, round(s) of biopanning. Any number of rounds of AAV selection may be performed. The total number of unique capsid variants may drop by a fold amount across AAV selection rounds. Capsid libraries may be pooled or combined at any step with any other capsid libraries described herein.

Following RNA recovery and PCR amplification, a systematic enrichment analysis by NGS is performed. Capsids enrichment ratio including comparison to a benchmark and sequence convergence is evaluated.

Peptide library candidates are evaluated and optimized using any of the methods described herein and are carried out, e.g., using methods similar, or equivalent, to those described in WO2020072683, the contents of which are herein incorporated by reference in their entirety, particularly the subject matter of Examples, 8, 9, and 10. The top-ranking peptide variants are generated and transduction efficacy evaluated as described in WO2020072683, the contents of which are herein incorporated by reference in their entirety, particularly the subject matter of Examples 10, 12 and 13.

### Example 3. Identification and Design of Orthogonal Evolution AAV Capsid Libraries

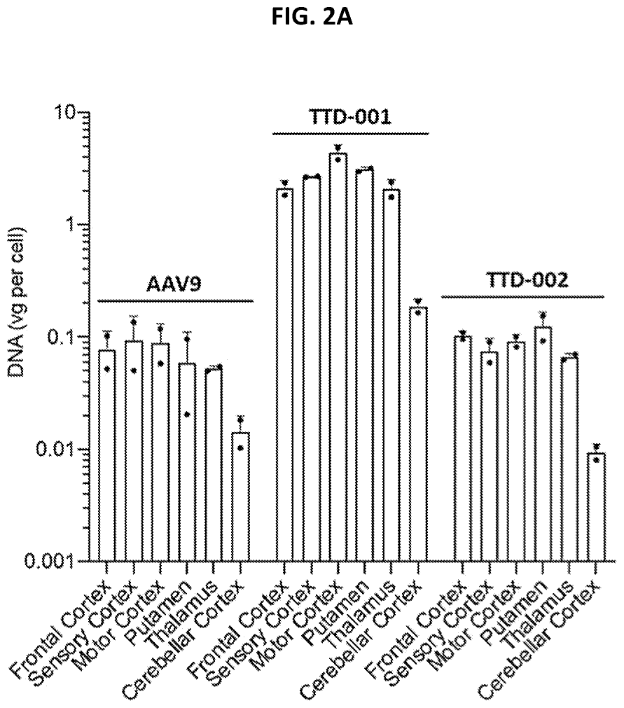

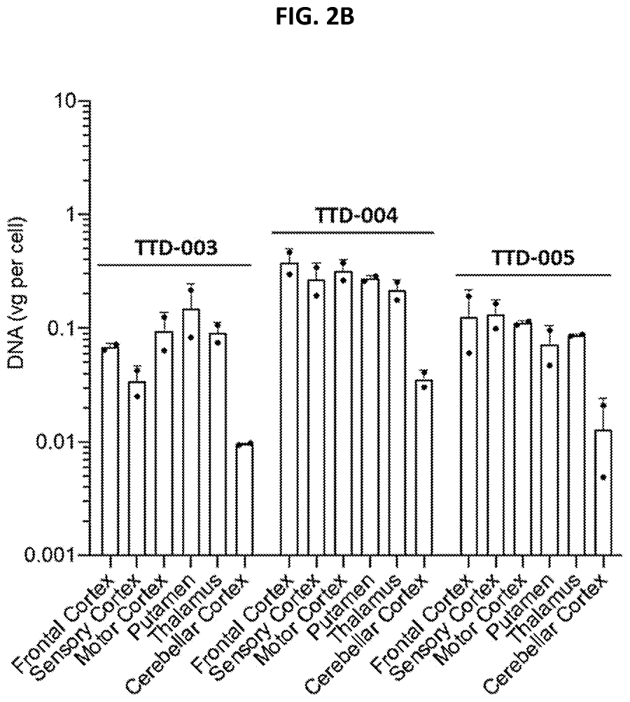

This study involves the use of orthogonal evolution wherein AAV particles may be administered for a first round of AAV selection across a set of any number of cell- and/or subject-types that may be from different species and/or strains; and, wherein any number of additional, i.e., subsequent, AAV selection rounds are performed either across a set of any number of cell- and/or subject-types that may be from different species and/or strains, or across a set of any number of cell- and/or subject-types that may be from the same species and/or strains, as represented in FIG. 2.

A TRACER based RNA-driven library selection for increased nervous system tissue transduction a set of any number of cell- and/or subject-types that may be from different species and/or strain is developed and carried out in accordance with methods similar, or equivalent, to those described in WO2020072683, the contents of which are herein incorporated by reference in their entirety, particularly the subject matter of Example 7. AAV libraries, e.g., AAV9 libraries, generated are administered for a first round of AAV selection (biopanning) by any route to a non-human primate (NHP), a rodent (e.g., a rat), and/or a cell (e.g., a human brain microvascular endothelial cell, or hBMVEC) at a given VG dose(s) per subject and/or cell. A number of groups of NHPs, rodents, and/or cells are administered promoter-driven (e.g., SYN-driven or GFAP-driven) libraries derived from wild-type AAV9 sequences, while other groups receive pooled libraries containing wild-type, PHP.eB-derived, or other AAV serotype sequences. After a period, RNA is extracted from a tissue, such as but not limited to spinal cord and brain tissue. The RNA preparation is subjected to mRNA enrichment. The enriched mRNA is reverse transcribed to cDNA. The cDNA is amplified. This method allows recovery of abundant amplicons from tissue samples.

Full-length capsid amplicons are used as templates for NGS library generation, as well as cloning into DNA libraries for the next, or subsequent round(s) of biopanning. Subsequent rounds of biopanning are performed either across a set of any number of cell- and/or subject-types that may be from different species and/or strain as used in the above-described first round, or across a set of any number of cell- and/or subject-types that may be from the same species and/or strain as used in the above-described first round. Any number of rounds of selection is performed. The total number of unique capsid variants may drop by a fold amount across AAV selection rounds. Capsid libraries may be pooled or combined at any step with any other capsid libraries described herein.

Following RNA recovery and PCR amplification, a systematic enrichment analysis by NGS is performed. Capsids enrichment ratio including comparison to a benchmark and sequence convergence is evaluated.

Peptide library candidates are evaluated and optimized using any of the methods described herein and are carried out, e.g., using methods similar, or equivalent, to those described in WO2020072683, the contents of which are herein incorporated by reference in their entirety. The top-ranking peptide variants are generated and transduction efficacy evaluated as in WO2020072683.

### Example 4. NHP high-throughput screen of TRACER AAV libraries

A TRACER based method as described in WO2020072683, the contents of which are herein incorporated by reference in their entirety, was adapted for use in non-human primates (NHP). An orthogonal evolution approach (e.g., NHP and BMVEC) was combined with a high throughput screening by NGS in NHP. Briefly, AAV9 starting libraries, driven by synapsin or GFAP promoters were administered to non-human primate (NHP) intravenously for in vivo AAV selection (biopanning), performed iteratively. All libraries were injected intravenously at a dose of 1e14VG per animal (approximately 3e13 VG/kg). Orthogonally, biopanning was conducted in hBMVEC cells using the same starting libraries. In the second round of biopanning in NHP, only libraries driven by the synapsin promoter were used. After a period, (e.g., 1 month) RNA was extracted from nervous tissue, e.g., brain and spinal cord. Following RNA recovery and RT-PCR amplification, a systematic NGS enrichment analysis was performed and the peptides shown in Table 7 were identified. Capsids enrichment ratio, including calculating the ratio of, e.g., P2/P1 reads and comparison to a benchmark (e.g., AAV9) was evaluated.

Candidate library enrichment data in P3 NHP brain for the peptides identified, over benchmark AAV9, are shown in Table 7. Data are provided as fold enrichment. Fifty-one variants showed greater than 10-fold enrichment over AAV9. Variants with 0.0 enrichment over AAV9 are not included in Table 7.

TABLE 7
NHP NGSAAV9 Enrichment

| | | Fold |
|---|---|---|
| | SEQ ID | enrichment |
| Peptide Sequence | NO: | over AAV9 |
| PLNGAVHLYA | 1725 | 473.7 |
| AQARDSPKGW | 1726 | 214 |
| LTNGAVRDRP | 1727 | 134.4 |
| VQAFTHDSRG | 1728 | 88.6 |
| AQAYSTDVRM | 1729 | 84.8 |
| AQAYSTDVRI | 1730 | 83.8 |
| AQAFTAAERM | 1731 | 74.9 |
| AQTHLQIGVA | 1732 | 54.6 |
| AQSNAVLSLA | 1733 | 51.6 |
| AQAYSTDERM | 1734 | 41.4 |
| AQAYSTDVRL | 1735 | 31.7 |
| AQATVSTLRM | 1736 | 31.5 |
| AQAYSTDERK | 1737 | 31.2 |
| AQAYSTDMRM | 1738 | 30.4 |
| VVNGAVLHVA | 1739 | 29.8 |
| AQAYSTDVTM | 1740 | 29.7 |
| AQAHLQIGVA | 1741 | 23 |
| FLDPAVSSKA | 1742 | 22.6 |
| AQAYVSTLRM | 1743 | 21.9 |
| AQAQTGPPLK | 1744 | 20.1 |
| EQASRLPTPG | 1745 | 20 |
| AQASVSTMRM | 1746 | 19.7 |
| TDYSAVRLGA | 1747 | 18 |
| TQAYSTDVRM | 1748 | 17.9 |
| AQALPSNERL | 1749 | 17.4 |
| AQAYSTDVRT | 1750 | 16.4 |
| AQSSLPEMVA | 1751 | 16.2 |
| AQAGEQSTRL | 1752 | 16.1 |
| AQASNDVGRA | 1753 | 15.4 |
| AQATFTASEY | 1754 | 15.3 |
| AKAHAGTIYS | 1755 | 14.9 |
| AQARTIDQCC | 1756 | 14.8 |
| AQEYNSNPKA | 1757 | 14.5 |
| AQVVDNSTHA | 1758 | 14.5 |
| AQATLSVPLK | 1759 | 14.4 |
| AQIVMNSLKA | 1760 | 12.5 |
| AQATMSQTMA | 1761 | 12.5 |
| AQALTQDERW | 1762 | 12 |
| AQAQLSTLRP | 1763 | 11.6 |
| AQVVMGISVA | 1764 | 11.4 |
| AQAYTTDVRM | 1765 | 11.4 |
| AQHIDSMRPP | 1766 | 11.3 |
| AQASTGTLRL | 1767 | 11.1 |
| AQHRALDYYA | 1768 | 11 |
| AQARESPRGL | 1769 | 10.9 |
| AQALLAGTRV | 1770 | 10.7 |
| TKIQAVPWNA | 1771 | 10.7 |
| AQASLSSTRP | 1772 | 10.6 |
| AQAMGSRSDQ | 1773 | 10.4 |
| AQAAQGTYRG | 1774 | 10.3 |
| SQENAVFSKA | 1775 | 10.3 |
| AQALSLSTRP | 1776 | 9.8 |
| AQAAAGTLRD | 1777 | 9.7 |
| AQASRLPTPG | 1778 | 9.5 |
| AQAGSLSERG | 1779 | 9.5 |
| AQSKGDGFTA | 1780 | 9.4 |
| GAGTAVTATA | 1781 | 9.3 |
| AQAQGSSSVG | 1782 | 8.8 |
| AQAYSTDARM | 1783 | 8.8 |
| ERAHAVTGLA | 1784 | 8.5 |
| AQAYGLPKGP | 1785 | 8.4 |
| AQAYSTEVRM | 1786 | 8.4 |
| AQAGVSTALH | 1787 | 8.2 |
| AQSYSTDVRM | 1788 | 8.1 |
| AQPLMSHTDA | 1789 | 7.9 |
| AQAAALASRP | 1790 | 7.9 |
| AQAAITSTIS | 1791 | 7.8 |
| AQPANDGLRA | 1792 | 7.5 |
| AQDYSTDVRM | 1793 | 7.4 |
| AQATLGYSTA | 1794 | 7.4 |
| AQATLGTIRV | 1795 | 7.3 |
| AQAGASDMVH | 1796 | 7 |
| AQAVSGTVRS | 1797 | 6.9 |
| GGTLAVVSLA | 1798 | 6.9 |
| AQAYSADVRM | 1799 | 6.8 |
| AQAFAMPKGL | 1800 | 6.6 |
| AQALVSTSRP | 1801 | 6.6 |
| AQASFQQAST | 1802 | 6.6 |
| AQAMTGNDRS | 1803 | 6.3 |
| AQASTQSPPG | 1804 | 6.1 |
| NARSAVESLA | 1805 | 6.1 |
| AQITVSHTTA | 1806 | 6 |
| AQALAGYDKA | 1807 | 6 |
| AQSTSHDTRA | 1808 | 5.9 |
| AQAIQDRTVV | 1809 | 5.8 |
| AQSKTTLTLA | 1810 | 5.7 |
| AQASMGTVRL | 1811 | 5.7 |
| AQHSDTLTRA | 1812 | 5.5 |
| AQKEMYTSVA | 1813 | 5.5 |
| AQASPSQPLL | 1814 | 5.4 |
| AQAYAGTIYS | 1815 | 5.3 |
| AQARSLEPVI | 1816 | 5.3 |
| TQAGVSTAVH | 1817 | 5.2 |
| AQNTLSLSLA | 1818 | 5.2 |
| AQAYVSSVKM | 1819 | 5.2 |
| AQAATSPRLG | 1820 | 5.1 |
| GYLTAVQPQA | 1821 | 5 |
| LNNLAVGMTA | 1822 | 5 |
| AQTVSVHVRA | 1823 | 5 |
| AQINGLVTTA | 1824 | 4.9 |
| AQAAITTTIS | 1825 | 4.8 |
| AQASTFVTTI | 1826 | 4.7 |
| AQALYDNVPL | 1827 | 4.6 |
| AQAAAGTWKG | 1828 | 4.6 |
| AQATTGTLRS | 1829 | 4.6 |
| GQYAADSSYA | 1830 | 4.5 |
| AQAGIATVRT | 1831 | 4.5 |
| AQALGHELRA | 1832 | 4.5 |
| AQAREAIPQG | 1833 | 4.4 |
| AQAMSGTLRM | 1834 | 4.4 |
| AQAVDRVRPP | 1835 | 4.4 |
| AQAPVNNDRG | 1836 | 4.3 |
| AQAQQVAGTM | 1837 | 4.3 |
| AQAEPRDTRA | 1838 | 4.3 |
| AQRLSEQGVA | 1839 | 4.2 |
| AQASEGIQLS | 1840 | 4.1 |
| AQRQGPDPLA | 1841 | 4.1 |
| AQVTLGSAKA | 1842 | 4.1 |
| AQAGASLGLA | 1843 | 4 |
| AQAFTQDERW | 1844 | 4 |
| AQASQTTVRS | 1845 | 4 |
| AQARVSSNGV | 1846 | 3.9 |
| AQGPLSGLRA | 1847 | 3.9 |
| AQAYGGQSLG | 1848 | 3.9 |
| AQANLGTVRQ | 1849 | 3.9 |
| AQARSDTRGL | 1850 | 3.8 |
| AQAGSDGPRL | 1851 | 3.8 |
| TDGAAVVMRA | 1852 | 3.8 |
| SGITAVPLHA | 1853 | 3.8 |
| AQAAAVGHLP | 1854 | 3.7 |
| AQRVEPKWIA | 1855 | 3.7 |
| AQAVASSPYA | 1856 | 3.7 |
| TQYGAVEGQD | 1857 | 3.7 |
| AQAKSHTLEG | 1858 | 3.6 |
| AQTHLQIVVA | 1859 | 3.6 |
| AQKNEHGMLA | 1860 | 3.6 |
| AQITVSHTRA | 1861 | 3.6 |
| AQARLAPKGL | 1862 | 3.6 |
| KTPGAVSTTA | 1863 | 3.6 |
| AQAFSGTIKS | 1864 | 3.6 |
| PLNGAVNLYA | 1865 | 3.5 |
| AQALTQDERC | 1866 | 3.5 |
| AQATAQVQRS | 1867 | 3.4 |
| AQTPALINLA | 1868 | 3.4 |
| AQASDRSPLL | 1869 | 3.4 |
| AQITVSHTMA | 1870 | 3.3 |
| AQATGTHLMG | 1871 | 3.3 |
| LDGGAVVVTA | 1872 | 3.3 |
| LTNGAVRDRA | 1873 | 3.2 |
| AQARGSDLRD | 1874 | 3.2 |
| AQATFGTQRI | 1875 | 3.2 |
| AQALPQTNRP | 1876 | 3.1 |
| AQARSNDPVL | 1877 | 3.1 |
| AQAYLAVQNG | 1878 | 3.1 |
| AQATQSTLRP | 1879 | 3.1 |
| AQALGGFGPQ | 1880 | 3.1 |
| LVGQAVGSRA | 1881 | 3.1 |
| AQSIANVVVA | 1882 | 3 |
| AQASPSVSRP | 1883 | 3 |
| AQTVVVSTTA | 1884 | 3 |
| VKEQAVSVMA | 1885 | 3 |
| AQQATGTFRA | 1886 | 3 |
| AQAQGSSSGG | 1887 | 3 |
| AQAHAVGPQG | 1888 | 3 |
| AQRLETKETA | 1889 | 3 |
| AQLAQGIGVA | 1890 | 3 |
| AQAVQSSFTI | 1891 | 3 |
| AQATYTASEY | 1892 | 2.9 |
| AQTSSQNLKA | 1893 | 2.9 |
| AQLVPSVAMA | 1894 | 2.9 |
| AQASPSAFAG | 1895 | 2.9 |
| AQALALVSAS | 1896 | 2.9 |
| AQASVGTTYT | 1897 | 2.9 |
| AQARVSSSGT | 1898 | 2.9 |
| NSMGAVLGAA | 1899 | 2.9 |
| AQHTDTLTRA | 1900 | 2.9 |
| AQPNLQPRGA | 1901 | 2.8 |
| AQADRHSSIV | 1902 | 2.8 |
| SPSVAVPSQA | 1903 | 2.8 |
| AQPGIVSTIA | 1904 | 2.8 |
| AQAQHSVGLP | 1905 | 2.7 |
| AQTNSGAILA | 1906 | 2.7 |
| AQDSYDVGRA | 1907 | 2.7 |
| EQAQGSSSVG | 1908 | 2.7 |
| AQAGVSTAVQ | 1909 | 2.7 |
| AQARDMLPLQ | 1910 | 2.7 |
| AQAMVGTLRG | 1911 | 2.7 |
| AQPNVVSTLA | 1912 | 2.7 |
| AQAGHVVTSD | 1913 | 2.7 |
| AQAYTTDERM | 1914 | 2.7 |
| TAVSAVQVMA | 1915 | 2.6 |
| AQAAAGTLRV | 1916 | 2.6 |
| VSNEAVHARA | 1917 | 2.6 |
| AQVLPQSLSA | 1918 | 2.6 |
| AQASVSTLRM | 1919 | 2.6 |
| AQAGLLLSVA | 1920 | 2.5 |
| AQANLVTGPL | 1921 | 2.5 |
| AQASQHSSMA | 1922 | 2.5 |
| GYSSAVSSVA | 1923 | 2.5 |
| AQVGVSPAVA | 1924 | 2.5 |
| DGTLAVPFKA | 1925 | 2.5 |
| AQAPPTSTAM | 1926 | 2.5 |
| AQATPANVRG | 1927 | 2.5 |
| AQAGSSNFLS | 1928 | 2.5 |
| AQLLAQDIRA | 1929 | 2.5 |
| AQPSSDGYRA | 1930 | 2.5 |
| AQALIGTLRT | 1931 | 2.5 |
| SVHGAVGILA | 1932 | 2.5 |
| AQPYVVSGAA | 1933 | 2.4 |
| AQWTHNITAA | 1934 | 2.4 |
| PTNAAVRTNA | 1935 | 2.4 |
| AHAYSTDVRM | 1936 | 2.4 |
| PLAAAVGMKA | 1937 | 2.4 |
| AQARDNSVML | 1938 | 2.4 |
| AQAQFPRNGG | 1939 | 2.4 |
| GALNAVNGVA | 1940 | 2.4 |
| AQASHQQGVP | 1941 | 2.4 |
| SYQSAVVPQA | 1942 | 2.3 |
| AQSIMGTIRA | 1943 | 2.3 |
| AQAYVSQAQG | 1944 | 2.3 |
| AQATGNQAHF | 1945 | 2.3 |
| AQVTVGTPIA | 1946 | 2.3 |
| AQAQTSTFRG | 1947 | 2.3 |
| SVHMAVTVSA | 1948 | 2.2 |
| AQAQSTLNLG | 1949 | 2.2 |
| AQDQTGPPLK | 1950 | 2.2 |
| HLAHAVSTAA | 1951 | 2.2 |
| AQALARDSSF | 1952 | 2.2 |
| AQLLSGTLKA | 1953 | 2.2 |
| AQASLLPTPG | 1954 | 2.2 |
| AQPMAGQSTA | 1955 | 2.2 |
| AQARSLEPDI | 1956 | 2.2 |
| AQFVTGNQDA | 1957 | 2.2 |
| AQATFKTSVP | 1958 | 2.1 |
| LNARAVEGPA | 1959 | 2.1 |
| AQALPNSGRP | 1960 | 2.1 |
| AQALNGSPEA | 1961 | 2.1 |
| AQATSLHPLP | 1962 | 2 |
| AQAVQPPLKN | 1963 | 2 |
| AQAMLSGTRI | 1964 | 2 |
| AQHVDLASKA | 1965 | 2 |
| AQASFATMRP | 1966 | 2 |
| AQAMPLNARS | 1967 | 2 |
| AQALVGQMRG | 1968 | 2 |
| VVNGAVLHLA | 1969 | 2 |
| AQAQTAPPLK | 1970 | 2 |
| AQGHGDLHRA | 1971 | 2 |
| AQAADRSPVH | 1972 | 2 |
| GALNAVTGVA | 1973 | 2 |
| AQAERMASLG | 1974 | 1.9 |
| AQAPPTTTRL | 1975 | 1.9 |
| AQAAVGQTLA | 1976 | 1.9 |
| AQSLGTGMHA | 1977 | 1.9 |
| AQSLGSPALA | 1978 | 1.9 |
| AQASVSVTRP | 1979 | 1.9 |
| AQATMSHTMA | 1980 | 1.9 |
| AQAVQSLTVG | 1981 | 1.9 |
| AQSQTGTYRA | 1982 | 1.9 |
| AQSLASVYAA | 1983 | 1.9 |
| STKLAVHEQA | 1984 | 1.9 |
| AQSHLFPTPA | 1985 | 1.9 |
| AQGTWSSSEA | 1986 | 1.9 |
| AQTPQGLTKA | 1987 | 1.9 |
| AQVSLGTQYA | 1988 | 1.9 |
| AQDSRLPTPG | 1989 | 1.8 |
| ASIQAVGVKA | 1990 | 1.8 |
| AQATMSEQRL | 1991 | 1.8 |
| TAQAAVQGMA | 1992 | 1.8 |
| AQAFNAAERM | 1993 | 1.8 |
| AQINFLSGVA | 1994 | 1.8 |
| PQHLAVSSEA | 1995 | 1.8 |
| AQALGNFPAV | 1996 | 1.8 |
| AQANASTVRV | 1997 | 1.8 |
| AQRIVDLTTA | 1998 | 1.8 |
| VRQVAVEGVA | 1999 | 1.8 |
| AQAPASSQKL | 2000 | 1.8 |
| AQQIDSMRPA | 2001 | 1.7 |
| AQAHGTSSLF | 2002 | 1.7 |
| AQVNSGIALA | 2003 | 1.7 |
| AQLHLAETRA | 2004 | 1.7 |
| AQALTHDERW | 2005 | 1.7 |
| NTVRAVIMEA | 2006 | 1.7 |
| AQAYVAGSRP | 2007 | 1.7 |
| AQDRAFVVSA | 2008 | 1.7 |
| AQAQEKQVFS | 2009 | 1.7 |
| AQACVSTAVH | 2010 | 1.7 |
| AQAFTHDSRG | 2011 | 1.7 |
| AQASHQGTVG | 2012 | 1.7 |
| AQAVLVTEQG | 2013 | 1.7 |
| AQAVVSTAVH | 2014 | 1.7 |
| AQATSRETKG | 2015 | 1.7 |
| YQQPAVSSRA | 2016 | 1.6 |
| AQANMGLSLS | 2017 | 1.6 |
| AQWTSSMSEA | 2018 | 1.6 |
| AQASISIMST | 2019 | 1.6 |
| AQASVAPLTC | 2020 | 1.6 |
| AQLVTVEKQA | 2021 | 1.6 |
| AQAATAGEKL | 2022 | 1.6 |
| AQALSHGPGG | 2023 | 1.6 |
| AQSNAHIEIA | 2024 | 1.6 |
| AQARSSSTGI | 2025 | 1.6 |
| AQAVGGDVTR | 2026 | 1.6 |
| AQAPRTVYQG | 2027 | 1.6 |
| AQALHNLGPA | 2028 | 1.6 |
| VRMGAVSDNA | 2029 | 1.6 |
| AQAFRTSQFT | 2030 | 1.6 |
| AQSSATMQRA | 2031 | 1.5 |
| AQTLAETYRA | 2032 | 1.5 |
| AQANGSIVLN | 2033 | 1.5 |
| AQARVADQLP | 2034 | 1.5 |
| AQAVKQGLYE | 2035 | 1.5 |
| AQAFSDGLKS | 2036 | 1.5 |
| AQVSVTPVKA | 2037 | 1.5 |
| VNGRAVSMMA | 2038 | 1.5 |
| SLVGAVAQMA | 2039 | 1.5 |
| AQARVSPVGL | 2040 | 1.5 |
| AQSNTTLTLA | 2041 | 1.5 |
| AQTSTEHLRA | 2042 | 1.5 |
| AQAGMGINLP | 2043 | 1.5 |
| AQANAHSLTL | 2044 | 1.5 |
| AQARFTTTEM | 2045 | 1.5 |
| AQLGYQEVKA | 2046 | 1.4 |
| AQAGQHASVF | 2047 | 1.4 |
| AQATGSNPRG | 2048 | 1.4 |
| AQAPVSPSIP | 2049 | 1.4 |
| AQTTLGVGTA | 2050 | 1.4 |
| AQASHLVSLA | 2051 | 1.4 |
| AQAPLTGLSV | 2052 | 1.4 |
| AQVSTSTLRA | 2053 | 1.4 |
| AQVQLGTLKA | 2054 | 1.4 |
| AQAHVSVSER | 2055 | 1.4 |
| AQLLLSGQTA | 2056 | 1.4 |
| TTSSAVLTPA | 2057 | 1.4 |
| AQFGADTVNA | 2058 | 1.4 |
| AQTFSSDNLA | 2059 | 1.4 |
| AQIHPANSRA | 2060 | 1.4 |
| AQSIGQFPVA | 2061 | 1.3 |
| AQVISPENLA | 2062 | 1.3 |
| AQALSAISAT | 2063 | 1.3 |
| AQAGVSASQM | 2064 | 1.3 |
| AQASTKTPLP | 2065 | 1.3 |
| AQAPPSTTAM | 2066 | 1.3 |
| AQAVSSDRMH | 2067 | 1.3 |
| AQAGSVTMRL | 2068 | 1.3 |
| AQAVLLGGAV | 2069 | 1.3 |
| AQAQRDMVTT | 2070 | 1.3 |
| AQAHHGSSLG | 2071 | 1.3 |
| VLSSAVGQRA | 2072 | 1.3 |
| AQAAGSVLLG | 2073 | 1.3 |
| AQAYPTDVRM | 2074 | 1.3 |
| AQWSRDAQSA | 2075 | 1.3 |
| AQQGLDMGRA | 2076 | 1.3 |
| AQAAQNHALV | 2077 | 1.3 |
| AQRSQIVEVA | 2078 | 1.3 |
| AQMSDVSGRA | 2079 | 1.3 |
| AKALTQDERW | 2080 | 1.3 |
| AQAVSSSTLT | 2081 | 1.3 |
| AQPSRLPTPG | 2082 | 1.3 |
| RTSTAVLDFA | 2083 | 1.3 |
| AQDLSSSIRA | 2084 | 1.3 |
| AQLLDGLTSA | 2085 | 1.3 |
| AQALIGLSKP | 2086 | 1.3 |
| AQASGTVRPP | 2087 | 1.2 |
| AQLLDTRYKA | 2088 | 1.2 |
| AQAPNTSFTA | 2089 | 1.2 |
| AQTHLQIGVD | 2090 | 1.2 |
| AQRLDTSQVA | 2091 | 1.2 |
| AQRTQDTLSA | 2092 | 1.2 |
| AQADIQSHAL | 2093 | 1.2 |
| PNMNAVGIKA | 2094 | 1.2 |
| AQAGVSTAVH | 2095 | 1.2 |
| AQASGKTFIG | 2096 | 1.2 |
| AQAGVQSTRL | 2097 | 1.2 |
| AQAQGAYPLV | 2098 | 1.2 |
| AQPYSTDVRM | 2099 | 1.2 |
| SSSVAVVTLA | 2100 | 1.2 |
| AQTYNGLNKA | 2101 | 1.2 |
| AQASVSKLRM | 2102 | 1.2 |
| AQRGSENEKA | 2103 | 1.2 |
| PITNAVLKTA | 2104 | 1.2 |
| TNSYAVSSPA | 2105 | 1.2 |
| PYQTAVAGAA | 2106 | 1.1 |
| GPALAVLGRA | 2107 | 1.1 |
| LSISAVPAKA | 2108 | 1.1 |
| AQTLGPLPHA | 2109 | 1.1 |
| AQAQQPLAHV | 2110 | 1.1 |
| AQTDGAWSKA | 2111 | 1.1 |
| AQALSGPPSI | 2112 | 1.1 |
| AQASSPSTRG | 2113 | 1.1 |
| AQASLASNRP | 2114 | 1.1 |
| AQNMALSTVA | 2115 | 1.1 |
| AQHSDTMTRA | 2116 | 1.1 |
| AQAMPRYPPL | 2117 | 1.1 |
| AQHIDSMSPA | 2118 | 1.1 |
| AQALPGTSRV | 2119 | 1.1 |
| AQAKSTQDVQ | 2120 | 1.1 |
| AQPLVSASKA | 2121 | 1.1 |
| AQAMSGTLRK | 2122 | 1.1 |
| AQTLILGAHA | 2123 | 1.1 |
| AQAGQARSQG | 2124 | 1.1 |
| AQRKDLSLVA | 2125 | 1.1 |
| AQALSAPMSL | 2126 | 1.1 |
| TNSLAVGMRA | 2127 | 1.1 |
| AQAPIGTVRP | 2128 | 1.1 |
| FIQAAVSSSA | 2129 | 1.1 |
| AQAEKPTHLL | 2130 | 1.1 |
| AQALSGDTTR | 2131 | 1.1 |
| AQAYIASGGT | 2132 | 1.1 |
| QLNQAVGTLA | 2133 | 1.1 |
| AQASGALDRP | 2134 | 1.1 |
| AQAQDTALRA | 2135 | 1.1 |
| AQAQAGMARG | 2136 | 1.1 |
| AQAQGSSAVG | 2137 | 1.1 |
| AQLLRDIGPA | 2138 | 1.1 |
| VDRGAVTQMA | 2139 | 1.1 |
| AQAVNVSKGS | 2140 | 1.1 |
| SVNTAVESLA | 2141 | 1.1 |
| AQARLPHTSS | 2142 | 1.1 |
| AQRNGSEVVA | 2143 | 1.1 |
| AQATDRVDRP | 2144 | 1.1 |
| AQASLSRERT | 2145 | 1.1 |
| AQAYSTHVRM | 2146 | 1.1 |
| AQHLSAGPTA | 2147 | 1.1 |
| LNGGAVSLRA | 2148 | 1 |
| AQAYGVSSVT | 2149 | 1 |
| AQFGSAVQLA | 2150 | 1 |
| AQAPPTSTRL | 2151 | 1 |
| AQVSTNWPKA | 2152 | 1 |
| AQTSTDLSRA | 2153 | 1 |
| AQAHSTDVRM | 2154 | 1 |
| AQATLTGHVS | 2155 | 1 |
| AQATTQGALT | 2156 | 1 |
| AQAAKASDRT | 2157 | 1 |
| AQGNEHGGRA | 2158 | 1 |
| AQALSTSLLL | 2159 | 1 |
| AQASLGSTYL | 2160 | 1 |
| AQAFSTVGAV | 2161 | 1 |
| AQLNGLVTTA | 2162 | 1 |
| AQASVRTLRM | 2163 | 1 |
| AQATMSRPWQ | 2164 | 1 |
| AQSSLPAMVA | 2165 | 1 |
| RETVAVGQYA | 2166 | 1 |
| AQAFGSEGRS | 2167 | 1 |
| SSGTAVEHRA | 2168 | 1 |
| FGTNAVIPRA | 2169 | 1 |
| AQAGQARSLG | 2170 | 1 |
| AQSFSSDNMA | 2171 | 1 |
| AQMNGLTGKA | 2172 | 1 |
| AQQNGKQHLA | 2173 | 1 |
| AQHIDSIRPA | 2174 | 1 |
| AQAADRLSTL | 2175 | 1 |
| AQFGLKDIRA | 2176 | 1 |
| AQAHQGGATL | 2177 | 1 |
| AQATYNSPKP | 2178 | 1 |
| AQAMSNMLRN | 2179 | 0.9 |
| VPISAVMSTA | 2180 | 0.9 |
| AQHSLGNTVA | 2181 | 0.9 |
| AQATSALSRL | 2182 | 0.9 |
| AQADRQTFPV | 2183 | 0.9 |
| AQAVNSMSIG | 2184 | 0.9 |
| AQALAIVSKN | 2185 | 0.9 |
| AQGQLQERFA | 2186 | 0.9 |
| AQFNGASAHA | 2187 | 0.9 |
| AQLGGQSPVA | 2188 | 0.9 |
| AQANGAYTDN | 2189 | 0.9 |
| AQNLSSSEPA | 2190 | 0.9 |
| AQSAIVLTTA | 2191 | 0.9 |
| ITRSAVPDVA | 2192 | 0.9 |
| GALKAVTGVA | 2193 | 0.9 |
| AQAVGQDYLR | 2194 | 0.9 |
| AQVTLNTPLA | 2195 | 0.9 |
| AQALTQDDRW | 2196 | 0.9 |
| AQAYSTNVRM | 2197 | 0.9 |
| AQAVAAPASL | 2198 | 0.9 |
| AQANPVSIMS | 2199 | 0.9 |
| AQASMQAVKD | 2200 | 0.9 |
| AQAVGGHSVA | 2201 | 0.9 |
| AQAVAASTRL | 2202 | 0.9 |
| GGTHAVSSFA | 2203 | 0.9 |
| AQAADSSGFR | 2204 | 0.9 |
| AQQSHVPQTA | 2205 | 0.9 |
| AQARVGNTNV | 2206 | 0.9 |
| AQTVSYSDLA | 2207 | 0.9 |
| AQAEHGLARS | 2208 | 0.9 |
| AQASNYPVAA | 2209 | 0.9 |
| VLLSAVGMAA | 2210 | 0.9 |
| AQALSGQNRG | 2211 | 0.9 |
| AQAWGQETRQ | 2212 | 0.9 |
| AQGYSTDVRM | 2213 | 0.9 |
| AQAGSVMSRE | 2214 | 0.8 |
| AQAGSLSARG | 2215 | 0.8 |
| AQATALAPKS | 2216 | 0.8 |
| AQAIRQNGSS | 2217 | 0.8 |
| AQLQDNLQLA | 2218 | 0.8 |
| AKAYSTDVRM | 2219 | 0.8 |
| AQSVDRTLLA | 2220 | 0.8 |
| AQAGQNSRLP | 2221 | 0.8 |
| AQTNLQPRGA | 2222 | 0.8 |
| PNTIAVGQRA | 2223 | 0.8 |
| AQAHATLSLS | 2224 | 0.8 |
| NHLRAVGSPA | 2225 | 0.8 |
| AQETDRNLRA | 2226 | 0.8 |
| AQARAETSGS | 2227 | 0.8 |
| AQHRELDSYA | 2228 | 0.8 |
| AQRHTSDVLA | 2229 | 0.8 |
| AQVGQTSSWA | 2230 | 0.8 |
| AQANSAALLM | 2231 | 0.8 |
| AQAIIERTAT | 2232 | 0.8 |
| AQSSRYEEKA | 2233 | 0.8 |
| QATGAVNPRA | 2234 | 0.8 |
| AQASYSVSVG | 2235 | 0.8 |
| AQQGHTVNNA | 2236 | 0.8 |
| AQASLPISTR | 2237 | 0.8 |
| AQHIDSMRPT | 2238 | 0.8 |
| AQAQDTENMR | 2239 | 0.8 |
| RTGAAVTGAA | 2240 | 0.8 |
| AQASSVRGMG | 2241 | 0.8 |
| AQPGIESTIA | 2242 | 0.8 |
| AQHVDLDSKA | 2243 | 0.8 |
| AQATTVPALG | 2244 | 0.8 |
| AQVNPTPQKA | 2245 | 0.8 |
| AQAGYISSAS | 2246 | 0.8 |
| AQTLILGDPA | 2247 | 0.8 |
| AQAASGLTMM | 2248 | 0.8 |
| AQALERPPSG | 2249 | 0.8 |
| TTYDAVHSKA | 2250 | 0.8 |
| AQAMLDSANG | 2251 | 0.7 |
| AQAMHQTDKF | 2252 | 0.7 |
| KSTVAVQSVA | 2253 | 0.7 |
| AQATAGTLIG | 2254 | 0.7 |
| GLKSAVTHVA | 2255 | 0.7 |
| AQAHSAYQGA | 2256 | 0.7 |
| AQSFSSDNLA | 2257 | 0.7 |
| AQLNMAASVA | 2258 | 0.7 |
| AQFSQAYNAA | 2259 | 0.7 |
| AQHLTAGLRA | 2260 | 0.7 |
| AQAHTVSPHL | 2261 | 0.7 |
| GILGAVLPRA | 2262 | 0.7 |
| AQHNSSSLLA | 2263 | 0.7 |
| AQAPQVAGTM | 2264 | 0.7 |
| AQHQDSRPMA | 2265 | 0.7 |
| AQRFQETGLA | 2266 | 0.7 |
| AQLTVSHTRA | 2267 | 0.7 |
| AQANLRTTMG | 2268 | 0.7 |
| AQAGLRDPRM | 2269 | 0.7 |
| AQHLLHGTAA | 2270 | 0.7 |
| RNQGAVASLA | 2271 | 0.7 |
| AQAGSSSVTW | 2272 | 0.7 |
| AQPHLQIGVA | 2273 | 0.7 |
| AQANSGAVLA | 2274 | 0.7 |
| AQLLGDAVKA | 2275 | 0.7 |
| SSGNAVSSLA | 2276 | 0.7 |
| AQVSVTMALA | 2277 | 0.7 |
| AQYHTRGFAA | 2278 | 0.7 |
| AQSTTKGTLA | 2279 | 0.7 |
| AQAQPPSARY | 2280 | 0.7 |
| AQAGLQGTAA | 2281 | 0.7 |
| AQPQGSSTFA | 2282 | 0.7 |
| TQYRAVEGQA | 2283 | 0.7 |
| AQAISTQLAG | 2284 | 0.7 |
| AQLGSNISHA | 2285 | 0.7 |
| AQTGLSGTVA | 2286 | 0.7 |
| AQRVDSSGRA | 2287 | 0.7 |
| AQAGLALNPN | 2288 | 0.7 |
| AQFYSDNSLA | 2289 | 0.7 |
| AQAVGAPQRL | 2290 | 0.7 |
| AQASYDDGRA | 2291 | 0.7 |
| SSFAAVATAA | 2292 | 0.6 |
| AQSTLSMPLA | 2293 | 0.6 |
| AQASLHAPRP | 2294 | 0.6 |
| HAVAAVSYPA | 2295 | 0.6 |
| AQTSPVMVQA | 2296 | 0.6 |
| AQADITSTIS | 2297 | 0.6 |
| AQAAGVAMLY | 2298 | 0.6 |
| AQASVSTLRK | 2299 | 0.6 |
| MDLKAVSSRA | 2300 | 0.6 |
| AQASLSTLRM | 2301 | 0.6 |
| AQPRSPLPMA | 2302 | 0.6 |
| GQYADVSSYA | 2303 | 0.6 |
| LVGGAVVVPA | 2304 | 0.6 |
| AQAQSARPLA | 2305 | 0.6 |
| AQSLHPSTTA | 2306 | 0.6 |
| AQFQTDLSRA | 2307 | 0.6 |
| RTELAVGLSA | 2308 | 0.6 |
| AQVVDNSPLA | 2309 | 0.6 |
| AQAVSSDSMH | 2310 | 0.6 |
| AQASPALHTL | 2311 | 0.6 |
| GQYAAVASYA | 2312 | 0.6 |
| AQLWQSRVDA | 2313 | 0.6 |
| GTFSAVQSTA | 2314 | 0.6 |
| AQAILSTIEV | 2315 | 0.6 |
| AQNVVSTLRA | 2316 | 0.6 |
| AQAMLAVSPG | 2317 | 0.6 |
| AQATDSLVAR | 2318 | 0.6 |
| AQASPQSSHG | 2319 | 0.6 |
| AQATPVHDTL | 2320 | 0.6 |
| LRSSAVGTAA | 2321 | 0.6 |
| MGRGAVLDTA | 2322 | 0.6 |
| AQSHLIPTPA | 2323 | 0.6 |
| AQAVLKAPIN | 2324 | 0.6 |
| AQKIAPAFLA | 2325 | 0.6 |
| AGNVAVLPHA | 2326 | 0.6 |
| AQSLGTGLHD | 2327 | 0.6 |
| AQAHAMSSRP | 2328 | 0.6 |
| AQQGKFDMRA | 2329 | 0.6 |
| AQALSGDGTR | 2330 | 0.6 |
| AQTHLQIAVA | 2331 | 0.6 |
| AQRTQGSSWA | 2332 | 0.6 |
| AIGSAVDLRA | 2333 | 0.6 |
| AQAQLASGTL | 2334 | 0.6 |
| AQALVSAGAL | 2335 | 0.6 |
| AQATESVPLK | 2336 | 0.6 |
| AQVYNSNPKA | 2337 | 0.6 |
| AQRTTYPSSA | 2338 | 0.6 |
| AQAMFQQAST | 2339 | 0.6 |
| AQPDALVIRA | 2340 | 0.6 |
| AQARDISMRG | 2341 | 0.6 |
| AQMSFGSTLA | 2342 | 0.6 |
| RLLSAVDQQA | 2343 | 0.6 |
| AQTTRSIENA | 2344 | 0.6 |
| AQVSDFSSRA | 2345 | 0.5 |
| AQAHSRVNTE | 2346 | 0.5 |
| AQAYSTDLRM | 2347 | 0.5 |
| AQALNGSAYS | 2348 | 0.5 |
| TQYGAVEAQA | 2349 | 0.5 |
| AQAHAGTIYS | 2350 | 0.5 |
| AQDPHSMRPA | 2351 | 0.5 |
| AQASANIHSS | 2352 | 0.5 |
| AQASLQAVSM | 2353 | 0.5 |
| AQSSHPAMVA | 2354 | 0.5 |
| AQANLQPRGA | 2355 | 0.5 |
| AQAVGSSPRG | 2356 | 0.5 |
| AQTSAPSALA | 2357 | 0.5 |
| AQAVQLQNRG | 2358 | 0.5 |
| AQAQGSGMVS | 2359 | 0.5 |
| AQAYPSSKSG | 2360 | 0.5 |
| AQAVSDYGRG | 2361 | 0.5 |
| AQMSLGATRA | 2362 | 0.5 |
| AQAFLNSASA | 2363 | 0.5 |
| AQALPSNARL | 2364 | 0.5 |
| AQANVSVRRE | 2365 | 0.5 |
| AQAGASVMVH | 2366 | 0.5 |
| AQSLAKDQSA | 2367 | 0.5 |
| AQILSALSSA | 2368 | 0.5 |
| AQSVHLSLAA | 2369 | 0.5 |
| AQALSASSFL | 2370 | 0.5 |
| AQTSQLNQTA | 2371 | 0.5 |
| AQSNLFPTPA | 2372 | 0.5 |
| AQAHGRSFDT | 2373 | 0.5 |
| AQLGSNTILA | 2374 | 0.5 |
| AQASMNSAKA | 2375 | 0.5 |
| AQRQAVEQSA | 2376 | 0.5 |
| AQASTGTLRH | 2377 | 0.5 |
| AQGPTYPNVA | 2378 | 0.5 |
| AQAGPTTSKA | 2379 | 0.5 |
| AQATTYRGMA | 2380 | 0.5 |
| AQVTNRGMPA | 2381 | 0.5 |
| AQAISGQAAW | 2382 | 0.5 |
| AQAFRGEDKG | 2383 | 0.5 |
| AQQSMPRFVA | 2384 | 0.5 |
| AQAGVKSTRL | 2385 | 0.5 |
| AQATGSILLA | 2386 | 0.5 |
| AQASGHSSFS | 2387 | 0.5 |
| AQTANDGLRA | 2388 | 0.5 |
| AQASQLALLA | 2389 | 0.5 |
| AQLVDRVPRA | 2390 | 0.5 |
| AQHSNGYVHA | 2391 | 0.5 |
| AQAAPSSSDS | 2392 | 0.5 |
| AQAMQRSSSA | 2393 | 0.5 |
| AQAASGRPTC | 2394 | 0.5 |
| AQPRPGDSRA | 2395 | 0.5 |
| AQRDRANGIA | 2396 | 0.5 |
| AQVLAISLSA | 2397 | 0.5 |
| AQAGMRDPRM | 2398 | 0.5 |
| AQASSNSSRA | 2399 | 0.5 |
| MHRDAVSGVA | 2400 | 0.5 |
| AQAEMKNMPP | 2401 | 0.5 |
| AQSGSLLASA | 2402 | 0.5 |
| AQAFASQSRG | 2403 | 0.5 |
| AQALHLPTLQ | 2404 | 0.5 |
| AQAKTGGMNT | 2405 | 0.5 |
| ISLNAVSGKA | 2406 | 0.5 |
| AQGVHGHYVA | 2407 | 0.5 |
| AQAYSKDVRM | 2408 | 0.5 |
| VPSIAVSSHA | 2409 | 0.5 |
| AQSSRHDDLA | 2410 | 0.5 |
| AQANGSGSRG | 2411 | 0.5 |
| AQVGIADRRA | 2412 | 0.5 |
| AQARGMESML | 2413 | 0.5 |
| AQAGVSTAGH | 2414 | 0.5 |
| AQVSTRNLIA | 2415 | 0.5 |
| AQAVPRLTAG | 2416 | 0.5 |
| AQRHMELQEA | 2417 | 0.5 |
| SQSRAVVWEA | 2418 | 0.5 |
| QSHTAVSSLA | 2419 | 0.5 |
| AKASVSTLRM | 2420 | 0.5 |
| AQASGSSQWA | 2421 | 0.5 |
| AQPNAQYMKA | 2422 | 0.5 |
| AQAMGTGSSL | 2423 | 0.5 |
| AQAFSTSQLT | 2424 | 0.5 |
| AQAKDQSQRL | 2425 | 0.4 |
| AQVGGNGSRA | 2426 | 0.4 |
| AQANGASRAV | 2427 | 0.4 |
| QVNKAVLDFA | 2428 | 0.4 |
| AQETLSSTRA | 2429 | 0.4 |
| GVYGAVHSSA | 2430 | 0.4 |
| AQTITIENVA | 2431 | 0.4 |
| AQALMKIADG | 2432 | 0.4 |
| AQANVSLQAA | 2433 | 0.4 |
| AQSTTSHLRA | 2434 | 0.4 |
| AQLSNLVSVA | 2435 | 0.4 |
| AQANSTPTRQ | 2436 | 0.4 |
| AQQRGDRAAA | 2437 | 0.4 |
| AQARLGQSVG | 2438 | 0.4 |
| AQQLTYGSSA | 2439 | 0.4 |
| AQPAEKQYSA | 2440 | 0.4 |
| AQAMPRSRGD | 2441 | 0.4 |
| AQGLSGRALA | 2442 | 0.4 |
| AQARVTAVDA | 2443 | 0.4 |
| AQVGVSTAVA | 2444 | 0.4 |
| AQTGVTSAQA | 2445 | 0.4 |
| AQALVTSSEK | 2446 | 0.4 |
| AQASPHSSMA | 2447 | 0.4 |
| AQALTQDEMW | 2448 | 0.4 |
| AQAFSTQQRL | 2449 | 0.4 |
| AQAGSQVTQA | 2450 | 0.4 |
| AQQSTLALKA | 2451 | 0.4 |
| AQALNGSHAA | 2452 | 0.4 |
| AQATEGHLRS | 2453 | 0.4 |
| AQPMANMLMA | 2454 | 0.4 |
| PSTSAVSQKA | 2455 | 0.4 |
| AQAPPSSTEM | 2456 | 0.4 |
| AQRERVDLAA | 2457 | 0.4 |
| AQASVTLPRT | 2458 | 0.4 |
| AQAYPSSSKA | 2459 | 0.4 |
| AQAHSGSAIP | 2460 | 0.4 |
| AQSPSQSLKA | 2461 | 0.4 |
| AQATPPATSP | 2462 | 0.4 |
| CLGAAVNQCA | 2463 | 0.4 |
| VLGQAVRDKA | 2464 | 0.4 |
| AQAQKANNVG | 2465 | 0.4 |
| AQTLLPVNGA | 2466 | 0.4 |
| AQAHGTIQRG | 2467 | 0.4 |
| TVYTAVGVSA | 2468 | 0.4 |
| LGRGAVLDMA | 2469 | 0.4 |
| AQANVRSDQM | 2470 | 0.4 |
| AQARDSQKGW | 2471 | 0.4 |
| AQTPGSRSAA | 2472 | 0.4 |
| AQALPSNARQ | 2473 | 0.4 |
| AQASATSVVH | 2474 | 0.4 |
| AQINLGTMRA | 2475 | 0.4 |
| AQVYNNTSAA | 2476 | 0.4 |
| AQASANLTRG | 2477 | 0.4 |
| AQLRTDYTRA | 2478 | 0.4 |
| AQAYSTDVKM | 2479 | 0.4 |
| AQTSQLYQPA | 2480 | 0.4 |
| AQALTQEERW | 2481 | 0.4 |
| LPNGAVRDRA | 2482 | 0.4 |
| VTGSAVAGIA | 2483 | 0.4 |
| AQAFSTDVRM | 2484 | 0.4 |
| AQAHGPTSGV | 2485 | 0.4 |
| AQAGVGLPIA | 2486 | 0.4 |
| AQVNSGQARA | 2487 | 0.4 |
| AQAQTGPPMK | 2488 | 0.4 |
| AQARLAPVAC | 2489 | 0.3 |
| LSIGAVASMA | 2490 | 0.3 |
| AQAQDLGVMR | 2491 | 0.3 |
| PTGLAVTSPA | 2492 | 0.3 |
| AQSASTSWSA | 2493 | 0.3 |
| AQNGSNVRNA | 2494 | 0.3 |
| AQASISSSAT | 2495 | 0.3 |
| AQNSHAHLAA | 2496 | 0.3 |
| AQAVGVKQFF | 2497 | 0.3 |
| AQAHLSPTHA | 2498 | 0.3 |
| AQPAYGSSYA | 2499 | 0.3 |
| AQAHQARSGS | 2500 | 0.3 |
| AQAHTSPTQR | 2501 | 0.3 |
| AQAATPSSSR | 2502 | 0.3 |
| AQAHNSYPKV | 2503 | 0.3 |
| AQSSLGSSLA | 2504 | 0.3 |
| AQALSRSNVG | 2505 | 0.3 |
| AQASLSSLSG | 2506 | 0.3 |
| AQHGSSEFTA | 2507 | 0.3 |
| AQSALVAGVA | 2508 | 0.3 |
| AQASSSSLRP | 2509 | 0.3 |
| AQTARDTGFA | 2510 | 0.3 |
| KSVQAVRDPA | 2511 | 0.3 |
| AQAGSHSSVS | 2512 | 0.3 |
| VRAHAVTGLA | 2513 | 0.3 |
| ASHTAVGEFA | 2514 | 0.3 |
| AQIRSEWRDA | 2515 | 0.3 |
| AQQLARVSGA | 2516 | 0.3 |
| AQAAITSTNS | 2517 | 0.3 |
| AQARDAVQLP | 2518 | 0.3 |
| AQAKELVSTS | 2519 | 0.3 |
| AQGIAETLSA | 2520 | 0.3 |
| AQLGSGFSTA | 2521 | 0.3 |
| AQNAKELERA | 2522 | 0.3 |
| AQTHLQNGVA | 2523 | 0.3 |
| SGNLAVGTPA | 2524 | 0.3 |
| AQPSPGTSVA | 2525 | 0.3 |
| AQSSAAAGRA | 2526 | 0.3 |
| AQAGISAAIM | 2527 | 0.3 |
| AQALGYHQTG | 2528 | 0.3 |
| NAGQAVAARA | 2529 | 0.3 |
| AQPFGGSGYA | 2530 | 0.3 |
| AQAGSPSRLC | 2531 | 0.3 |
| AQARTIGTIA | 2532 | 0.3 |
| AQVVSVSSRA | 2533 | 0.3 |
| AQAGQARSMG | 2534 | 0.3 |
| AQATRGVTAG | 2535 | 0.3 |
| AQQSNGYGRA | 2536 | 0.3 |
| AQASLAPLKS | 2537 | 0.3 |
| AQPGANHNGA | 2538 | 0.3 |
| LGRGAVPDTA | 2539 | 0.3 |
| AQHFQTASLA | 2540 | 0.3 |
| AQAPAGHHTR | 2541 | 0.3 |
| AQPSVQNSMA | 2542 | 0.3 |
| AQAKLSGHVS | 2543 | 0.3 |
| AQFGTSSPSA | 2544 | 0.3 |
| AQASHISSVR | 2545 | 0.3 |
| AQALSRNGIG | 2546 | 0.3 |
| AQASAQVQRS | 2547 | 0.3 |
| AQGGPHLQAA | 2548 | 0.3 |
| AQAQSDSAFR | 2549 | 0.3 |
| AQTYSTDVRM | 2550 | 0.3 |
| LARGAVLDTA | 2551 | 0.3 |
| AQASPHTLRS | 2552 | 0.3 |
| AQHSDTQTRA | 2553 | 0.3 |
| AQATPSPSAS | 2554 | 0.3 |
| AQNQVTYSKA | 2555 | 0.3 |
| AQHTSVVYGA | 2556 | 0.3 |
| AQAQVSQMSH | 2557 | 0.3 |
| SFLRAVKNDA | 2558 | 0.3 |
| AQAYSTDVGM | 2559 | 0.3 |
| AKTPALINLA | 2560 | 0.3 |
| VSTAAVSSAA | 2561 | 0.3 |
| AQAPITSTIS | 2562 | 0.3 |
| AQTNLQTRGA | 2563 | 0.3 |
| AQATRLPTPG | 2564 | 0.3 |
| PQHLAVSSAA | 2565 | 0.2 |
| AQASPHPSRP | 2566 | 0.2 |
| AQAQPAGQRG | 2567 | 0.2 |
| AQPQRQGVQA | 2568 | 0.2 |
| AQHVAGSSNA | 2569 | 0.2 |
| AQVPIQMGVA | 2570 | 0.2 |
| AQATVSVPLK | 2571 | 0.2 |
| AQISVSHTRA | 2572 | 0.2 |
| SLVGPVAQMA | 2573 | 0.2 |
| AQPRLNLTEA | 2574 | 0.2 |
| AQASQEYSRL | 2575 | 0.2 |
| AQKSLAFDSA | 2576 | 0.2 |
| AQALGHSHHC | 2577 | 0.2 |
| AQAAQTGRPI | 2578 | 0.2 |
| AQASGTSVRQ | 2579 | 0.2 |
| AQAMGTASYC | 2580 | 0.2 |
| AQISHNHPQA | 2581 | 0.2 |
| AQAYSTYVRM | 2582 | 0.2 |
| AQVGKLDIRA | 2583 | 0.2 |
| AQLKQGGINA | 2584 | 0.2 |
| AQASAHFREP | 2585 | 0.2 |
| AQALDTVLSA | 2586 | 0.2 |
| GAGTAVGNIA | 2587 | 0.2 |
| AQANGSATYA | 2588 | 0.2 |
| AQTQLAQQKA | 2589 | 0.2 |
| AQYVTTVSPA | 2590 | 0.2 |
| SQFSAVTVTA | 2591 | 0.2 |
| AQAASDSFRY | 2592 | 0.2 |
| AQASPASVTR | 2593 | 0.2 |
| AQARDSGMFL | 2594 | 0.2 |
| AQSKTTLTLS | 2595 | 0.2 |
| AQLVQESLSA | 2596 | 0.2 |
| AQAALKSLAG | 2597 | 0.2 |
| AQAVPNQGQK | 2598 | 0.2 |
| AQALSRSSLG | 2599 | 0.2 |
| AQAGSVMSRV | 2600 | 0.2 |
| AQMATVTPMA | 2601 | 0.2 |
| AQARTASGID | 2602 | 0.2 |
| SHSSAVSHPA | 2603 | 0.2 |
| AQADRMRTTA | 2604 | 0.2 |
| AQNAQNRALA | 2605 | 0.2 |
| AQAASNAYSS | 2606 | 0.2 |
| AQATFQQAST | 2607 | 0.2 |
| AQVYTISTPA | 2608 | 0.2 |
| AQTVIAVGVA | 2609 | 0.2 |
| LARGAVPPTA | 2610 | 0.2 |
| AQMLQTSVLA | 2611 | 0.2 |
| AQARQVSPLL | 2612 | 0.2 |
| AQAGQMSNAR | 2613 | 0.2 |
| AQTPALIKLA | 2614 | 0.2 |
| AQAYTTDVRK | 2615 | 0.2 |
| VVKGAVLHVA | 2616 | 0.2 |
| AQDTVSVPLK | 2617 | 0.2 |
| AQKGAPSLQA | 2618 | 0.2 |
| AQASYDVGRA | 2619 | 0.2 |
| AQGPLSGMRA | 2620 | 0.2 |
| AQALGTSVPA | 2621 | 0.2 |
| AQVNKGASTA | 2622 | 0.2 |
| AQLTRTSPVA | 2623 | 0.2 |
| AQADAALRFS | 2624 | 0.2 |
| AQVQLVVSPA | 2625 | 0.2 |
| AQAYSSDVRM | 2626 | 0.2 |
| AQARSGLSLP | 2627 | 0.2 |
| AQNGHKFTAA | 2628 | 0.2 |
| AQGLSSATKA | 2629 | 0.2 |
| AQGTWSTVKA | 2630 | 0.2 |
| AQASGVGGRI | 2631 | 0.2 |
| AQTSYPAQKA | 2632 | 0.2 |
| AQNAVPTHSA | 2633 | 0.2 |
| AQSYPEITRA | 2634 | 0.2 |
| AQTGLSTSSA | 2635 | 0.2 |
| AQYDTHNFAA | 2636 | 0.2 |
| AQAVLSSVIQ | 2637 | 0.2 |
| AQDSAVALMA | 2638 | 0.2 |
| AQATGKGALP | 2639 | 0.2 |
| AQNSRSGHDA | 2640 | 0.2 |
| AQAFQKEPSV | 2641 | 0.2 |
| AQAGSTSGKM | 2642 | 0.2 |
| AQRDQAHSQA | 2643 | 0.2 |
| AQAASALSGR | 2644 | 0.2 |
| AQARHSSLLP | 2645 | 0.2 |
| AQGPGTSYLA | 2646 | 0.2 |
| FLAPAVSSKA | 2647 | 0.2 |
| AQAGPQCSSC | 2648 | 0.2 |
| AQALTQHERW | 2649 | 0.2 |
| AQAIRSSERV | 2650 | 0.2 |
| AQAVHSSSVY | 2651 | 0.2 |
| AQSSRTALAA | 2652 | 0.2 |
| AQITFSHTRA | 2653 | 0.2 |
| AQALTLSGGL | 2654 | 0.2 |
| AQAGKTLSVL | 2655 | 0.2 |
| AQASRSNLDN | 2656 | 0.2 |
| AQGSLSTHTA | 2657 | 0.2 |
| AQQSVAYNVA | 2658 | 0.2 |
| AQHTLRLSSA | 2659 | 0.2 |
| AQAGGTPNKL | 2660 | 0.2 |
| AQAFQSLTLA | 2661 | 0.2 |
| AQAVALSHQE | 2662 | 0.2 |
| AQMLASGIPA | 2663 | 0.2 |
| AQNRALDSYA | 2664 | 0.2 |
| AQASGSTTRN | 2665 | 0.2 |
| AQARGDGYVA | 2666 | 0.2 |
| DARVAVLDFA | 2667 | 0.2 |
| AQAVASQVSR | 2668 | 0.2 |
| AQARGPSPAT | 2669 | 0.2 |
| AQHRALDSYD | 2670 | 0.2 |
| AQLIDSTSRA | 2671 | 0.2 |
| AQAQTLSRGS | 2672 | 0.2 |
| AQFRSAITSA | 2673 | 0.2 |
| AQANMTKQSL | 2674 | 0.2 |
| AQNAGSTSRA | 2675 | 0.2 |
| VLGSAVTGRA | 2676 | 0.2 |
| AQPMLQSSSA | 2677 | 0.2 |
| AQLGTPSLSA | 2678 | 0.2 |
| AQATAHTGVP | 2679 | 0.2 |
| AQAVGRDNRL | 2680 | 0.2 |
| AQATSASVWA | 2681 | 0.2 |
| AQAGSEASLR | 2682 | 0.2 |
| AQANQNRTAF | 2683 | 0.2 |
| AQASAQVKRS | 2684 | 0.2 |
| AQATSGVHHP | 2685 | 0.2 |
| AQTHMQIGVA | 2686 | 0.2 |
| AQSHIFPTPA | 2687 | 0.2 |
| AQLFHTGSPA | 2688 | 0.2 |
| LASRAVVGTA | 2689 | 0.2 |
| AQALLRVGVG | 2690 | 0.2 |
| AQITLPSGTA | 2691 | 0.2 |
| AQAEKSLGRQ | 2692 | 0.2 |
| AQTSNTTTRA | 2693 | 0.2 |
| AQAHTQASYM | 2694 | 0.2 |
| AQERGASSSA | 2695 | 0.2 |
| AQATPSSTAM | 2696 | 0.2 |
| AQSTVNRTYA | 2697 | 0.2 |
| AQAGHGPSTR | 2698 | 0.2 |
| AQLSLVPLQA | 2699 | 0.2 |
| AQLHSPIPSA | 2700 | 0.2 |
| AQSLARDGLA | 2701 | 0.2 |
| AQAPPSSPAM | 2702 | 0.2 |
| TQYGAVERQA | 2703 | 0.1 |
| AQAGQARSLA | 2704 | 0.1 |
| AQPVGRVPPA | 2705 | 0.1 |
| AQAREQRGPV | 2706 | 0.1 |
| AQKTSLLWEA | 2707 | 0.1 |
| AQGSGKNLIA | 2708 | 0.1 |
| AQASEGHQLS | 2709 | 0.1 |
| AQALHAGHHP | 2710 | 0.1 |
| AQSKRDDPSA | 2711 | 0.1 |
| AQTSRELRMA | 2712 | 0.1 |
| AQALPASGAR | 2713 | 0.1 |
| AQSNALLSLA | 2714 | 0.1 |
| AQASPVVGVS | 2715 | 0.1 |
| AQARGDSYMA | 2716 | 0.1 |
| AQAGASSLTV | 2717 | 0.1 |
| AQALRPVNGT | 2718 | 0.1 |
| AQVRSGPTLA | 2719 | 0.1 |
| AQFPPLSRSA | 2720 | 0.1 |
| AQVARGTVQA | 2721 | 0.1 |
| AQTSTQSPPG | 2722 | 0.1 |
| AQARDGMNVR | 2723 | 0.1 |
| AQAVSRNVVV | 2724 | 0.1 |
| AQHTATRSVA | 2725 | 0.1 |
| AQAVREDGHA | 2726 | 0.1 |
| TNSSAVAASA | 2727 | 0.1 |
| AQATFQLAST | 2728 | 0.1 |
| AQAHHQQTSL | 2729 | 0.1 |
| AQGQHAHMMA | 2730 | 0.1 |
| AQATSSLHVL | 2731 | 0.1 |
| AQAPNSGLAM | 2732 | 0.1 |
| SASRAVLDFA | 2733 | 0.1 |
| AQARGEQRFV | 2734 | 0.1 |
| AQTHLQIRVA | 2735 | 0.1 |
| AQAPPSSKAM | 2736 | 0.1 |
| AQIVSKAMPA | 2737 | 0.1 |
| AQASVRNNPS | 2738 | 0.1 |
| AQAESRVAAL | 2739 | 0.1 |
| LTNGAVRDRT | 2740 | 0.1 |
| AQGRLAGSLA | 2741 | 0.1 |
| AQAGQDSARR | 2742 | 0.1 |
| AQAASRLGAV | 2743 | 0.1 |
| AQALARGMAS | 2744 | 0.1 |
| AQASRGLSMG | 2745 | 0.1 |
| AQAQASSYGS | 2746 | 0.1 |
| AKASRLPTPG | 2747 | 0.1 |
| AQSLSRASTA | 2748 | 0.1 |
| AQASTFVQTI | 2749 | 0.1 |
| AQASSKVVAA | 2750 | 0.1 |
| AQAYRNGEAA | 2751 | 0.1 |
| AQAYSTGVRM | 2752 | 0.1 |
| AQAVSSRSMG | 2753 | 0.1 |
| AQARGGLATP | 2754 | 0.1 |
| AQAGHSGVRA | 2755 | 0.1 |
| AQPSYHGGAA | 2756 | 0.1 |
| AQRVNQVSTA | 2757 | 0.1 |
| AQAAFQQAST | 2758 | 0.1 |
| AQAVPGSPRA | 2759 | 0.1 |
| AQLSLSPLAA | 2760 | 0.1 |
| AQANMTVRVS | 2761 | 0.1 |
| AQATRSSGDP | 2762 | 0.1 |
| AQVASNATRA | 2763 | 0.1 |
| AQTNQQPRGA | 2764 | 0.1 |
| AQRLQNDHLA | 2765 | 0.1 |
| AQAPVQLGRP | 2766 | 0.1 |
| AQRQGPDTLA | 2767 | 0.1 |
| AQHTLSNHMA | 2768 | 0.1 |
| AQLSGMVNRA | 2769 | 0.1 |
| AQDRQVSSRA | 2770 | 0.1 |
| AQRQLSTSLA | 2771 | 0.1 |
| AQQRPTVSFA | 2772 | 0.1 |
| AQAKPHSQLD | 2773 | 0.1 |
| AQAGRVNHPP | 2774 | 0.1 |
| AQAINSQSMR | 2775 | 0.1 |
| AQYSTAVMSA | 2776 | 0.1 |
| SQARAVERSA | 2777 | 0.1 |
| AQAYKSSSVG | 2778 | 0.1 |
| AQASTPGLYP | 2779 | 0.1 |
| AQSRTSMLAA | 2780 | 0.1 |
| AQLFSSNMPA | 2781 | 0.1 |
| AQAYCTDVRM | 2782 | 0.1 |
| AQTMSRGFVA | 2783 | 0.1 |
| AQALNGYPAA | 2784 | 0.1 |
| AQAQTGHPLK | 2785 | 0.1 |
| AQASSNSQYR | 2786 | 0.1 |
| AQAAIKSTIS | 2787 | 0.1 |
| AQSTLNLRPA | 2788 | 0.1 |
| AQATLSPGSG | 2789 | 0.1 |
| AQANGSGTGR | 2790 | 0.1 |
| STSLAVAGRA | 2791 | 0.1 |
| AQASNLSAYR | 2792 | 0.1 |
| AQASRQVLVA | 2793 | 0.1 |
| NEVRAVFFEA | 2794 | 0.1 |
| AKAQGSSSVG | 2795 | 0.1 |
| ARGSAVQSQA | 2796 | 0.1 |
| AVRVAVSSSA | 2797 | 0.1 |
| AQAFSTSQFK | 2798 | 0.1 |
| AQGTSSQRTA | 2799 | 0.1 |
| AQATMSQTIA | 2800 | 0.1 |
| AQSANRSTLA | 2801 | 0.1 |
| AQRDLAHSKA | 2802 | 0.1 |
| AQASKVGLYA | 2803 | 0.1 |
| AQAYYTDVRM | 2804 | 0.1 |
| AQAGLRDPRA | 2805 | 0.1 |
| AQAFSQATGA | 2806 | 0.1 |
| AQVAGMSVRA | 2807 | 0.1 |
| AQAGQSSFTI | 2808 | 0.1 |
| AQKEMRSQGA | 2809 | 0.1 |
| AQNYSSGVRA | 2810 | 0.1 |
| AQITVSYTRA | 2811 | 0.1 |
| AQAQQPRSSI | 2812 | 0.1 |
| WTSGAVPGKA | 2813 | 0.1 |
| AQFKPSQVIA | 2814 | 0.1 |
| RQGQAVGSSA | 2815 | 0.1 |
| AQSISPHYAA | 2816 | 0.1 |
| AQARSLNEYK | 2817 | 0.1 |
| AQAASSRLMA | 2818 | 0.1 |
| AQAYSTDGRM | 2819 | 0.1 |
| AQASVPRVMG | 2820 | 0.1 |
| AQGQMPRYPA | 2821 | 0.1 |
| AQASSGMKPC | 2822 | 0.1 |
| AQPLRSSLSA | 2823 | 0.1 |
| AQNSASQSQA | 2824 | 0.1 |
| AQGHLSGLRA | 2825 | 0.1 |
| AQRAQSGVAA | 2826 | 0.1 |
| AQANPRLQDK | 2827 | 0.1 |
| AQAPRTATLG | 2828 | 0.1 |
| AQRTASLSQA | 2829 | 0.1 |
| AQGNPGLLRA | 2830 | 0.1 |
| MSSHAVGNRA | 2831 | 0.1 |
| AQLAPKASPA | 2832 | 0.1 |
| AQTTQGRERA | 2833 | 0.1 |
| AQASGKSTSS | 2834 | 0.1 |
| AQAPHQHSMK | 2835 | 0.1 |

A subset of the peptide variants from the NHP biopanning showed a very strong and consistent enrichment over AAV9 and PHP.B controls. Further, the peptide of SEQ ID NO: 1725 not only showed a strong enrichment over AAV9 in the brain, but also in the spinal cord, as it led to a 125.6 fold enrichment over AAV9 in the spinal cord. Following the removal of the least reliable variants, a set of 22 variants with enrichment factors ranging from 7-fold to >400-fold over AAV9 was identified. These were cross-referenced to a non-synthetic PCR-amplified library screened in parallel and 12 candidates showed reliable enrichment and high consistency in both assays. Of these, 5 candidates with the highest enrichment scores in both assays and the highest consistency across animals and tissues were retained for individual evaluation. Candidate capsids were labeled TTD-001, TTD-002, TTD-003, TTD-004 and TTD-005 as shown in Table 3 above.

After 3 rounds of screening of AAV9 peptide insertion library in NHP, many capsids outperformed their parental capsid AAV9 in penetration of the blood brain barrier (BBB). Some of the capsids comprising a peptide showed high enrichment scores and high consistency both across different brain tissue samples from the same animal and across different animals. Consistency in both NNK and NNM codons was also observed. 22 capsid variants exhibited enrichment factors ranging from 7-fold to >400-fold over AAV9 in the brain tissues. A majority of these variants also demonstrated high enrichment factors up to 125-fold over AAV9 in the spinal cord. Of these, 5 candidates with diverse inserted sequences were selected for further evaluation as individual capsids.

### Example 5. Individual Capsid Characterization

The goal of these experiments was to determine the transduction level and the spatial distribution of each of the 5 capsid candidates selected from the study described in Example 4 relative to AAV9 following intravascular infusion in NHPs (cynomolgus macaque). The 5 selected capsid candidates were TTD-001 (SEQ ID NO: 3623 and 3636, comprising SEQ ID NO: 1725 or 3648), TTD-002 (SEQ ID NO: 3624, 3625, and 3637, comprising SEQ ID NO: 1726 or 3649), TTD-003 (SEQ ID NO: 3626 and 3638, comprising SEQ ID NO: 1729 or 3650), TTD-004 (SEQ ID NO: 3627 and 3639, comprising SEQ ID NO: 1760 or 3651) and TTD-005 (SEQ ID NO: 3628 and 3640, comprising SEQ ID NO: 1769 or 3652) as outlined in Table 3 above.

AAV particles were generated with each of these 5 capsids encapsulating a transgene encoding a payload fused to an HA tag (payload-HA) and driven by a full-length CMV/chicken beta actin promoter by triple transfection in HEK293T cells and formulated in a pharmaceutically acceptable solution.

Each test capsid and AAV9 control were tested by intravenously providing two (2) NHP females the AAV particle formulation at a dose of 2e13 VG/kg. The in-life period was 14 days and then a battery of CNS and peripheral tissues were collected for quantification of transgene mRNA, transgene protein and viral DNA (biodistribution). Samples were also collected, fixed and paraffin embedded for immunohistochemical stainings.

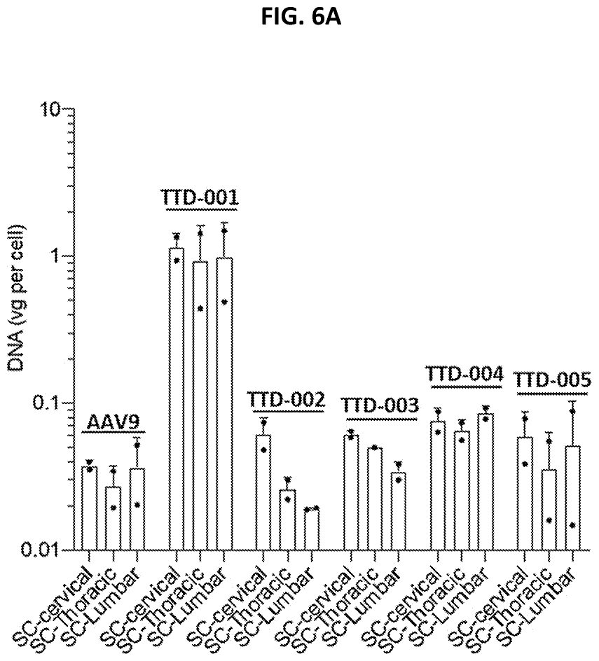

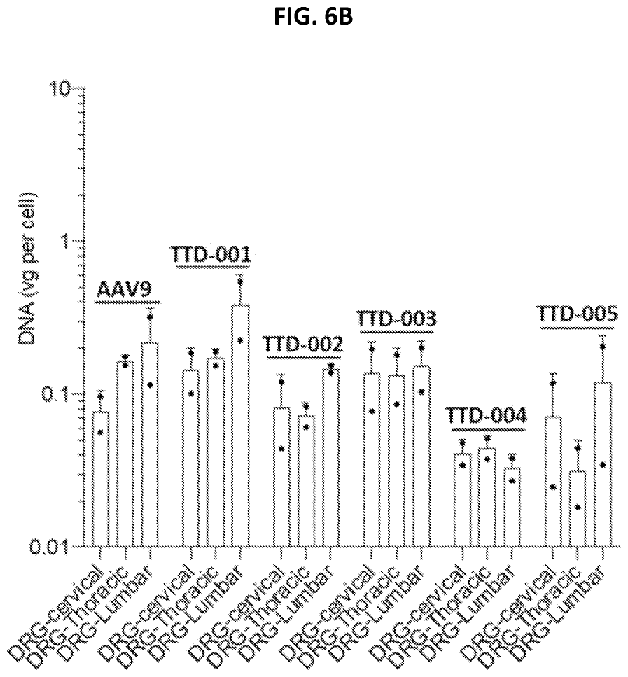

In a first pass screening of RNA quantification by qRT-PCR and RT-ddPCR, total RNA was extracted from 3-mm punches from various areas of the brain (cortex, striatum, hippocampus, cerebellum), spinal cord sections, liver and heart, and analyzed by qRT-PCR using a proprietary Taqman set specific for the synthetic CAG exon-exon junction. Cynomolgus TBP (TATA box-binding protein) was used as a housekeeping gene. Data are shown in FIG. 6A, FIG. 6B, and FIG. 6C of WO2021230987, the contents of which are hereby incorporated by reference in their entirety.

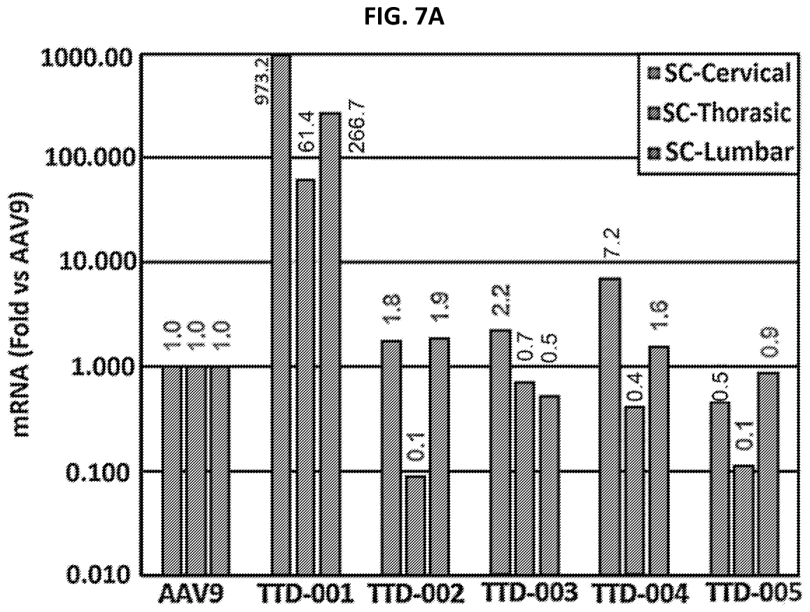

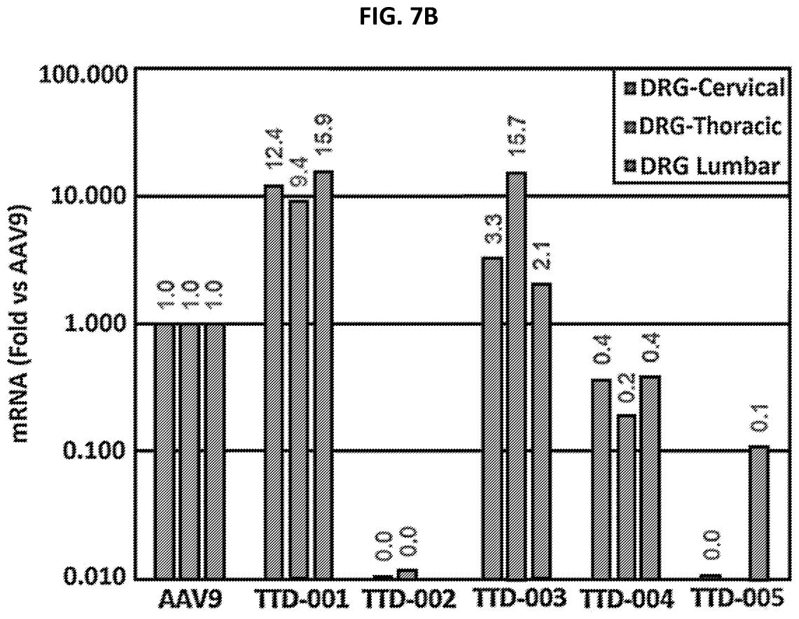

TRACER capsids showed an increase in RNA expression in all brain regions relative to AAV9 in at least one animal. The highest and most consistent increase in brain transduction was observed with capsids TTD-003 and TTD-004 (8- to 200-fold depending in various anatomical locations). In this initial screening TTD-001 was not assessed due to staggered animal dosing. An approximate 10- to 12-fold increase was consistently observed in whole brain slices (equivalent to an average of multiple regions), which was consistent with the values indicated in a next-generation sequencing (NGS) assay. In order to increase data robustness, droplet digital RT-PCR (ddPCR) was performed in parallel to qRT-PCR and confirmed the trends indicated by the qPCR data as shown in FIG. 7 of WO2021230987, the contents of which are hereby incorporated by reference in their entirety.

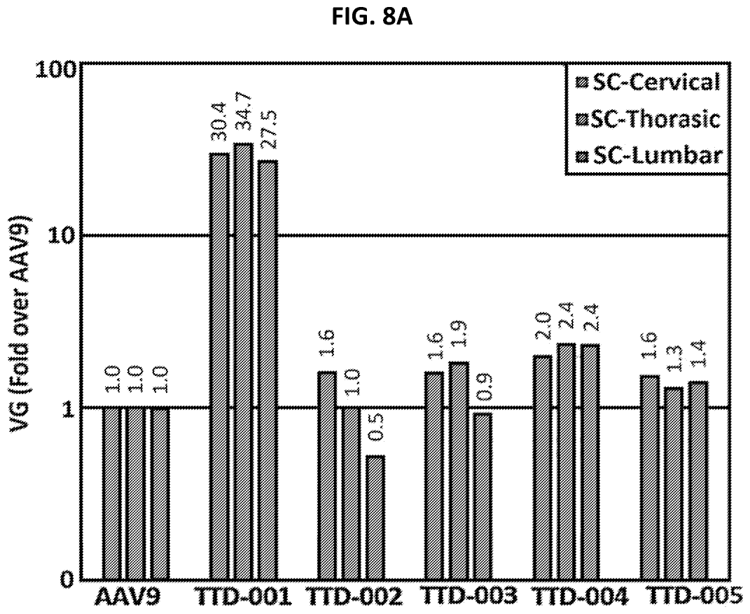

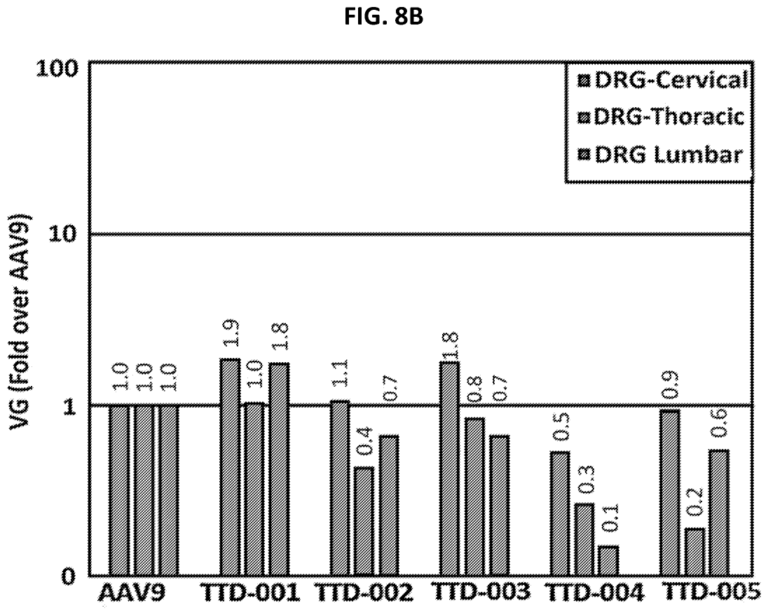

Interestingly, RNA quantification performed in the spinal cord and dorsal root ganglia indicated important differences between the capsid variants. The spinal cord transduction profile was consistent with the brain, with a strong and consistent increase with TTD-003 and TTD-004 capsids, but interestingly the DRG transduction suggested a substantial detargeting of the TTD-004 capsid, whereas the TTD-003 capsid showed a strongly increased RNA expression as shown in FIG. 8 of WO2021230987, the contents of which are hereby incorporated by reference in their entirety.

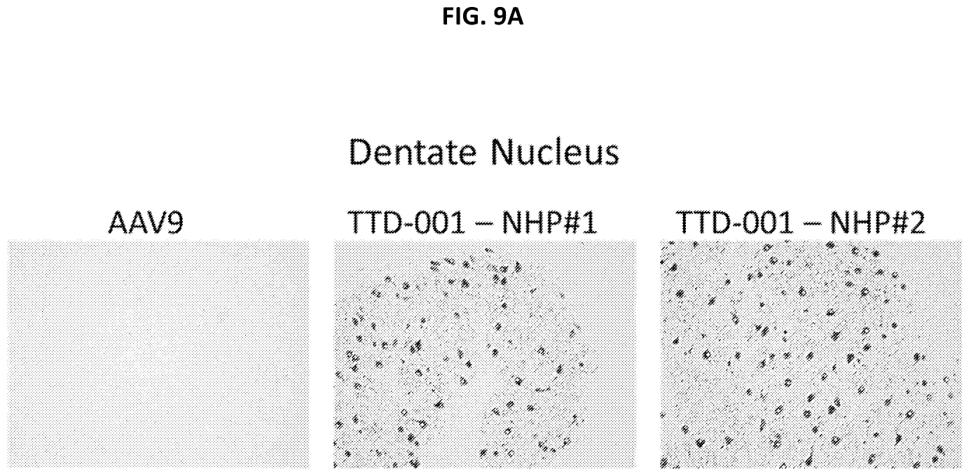

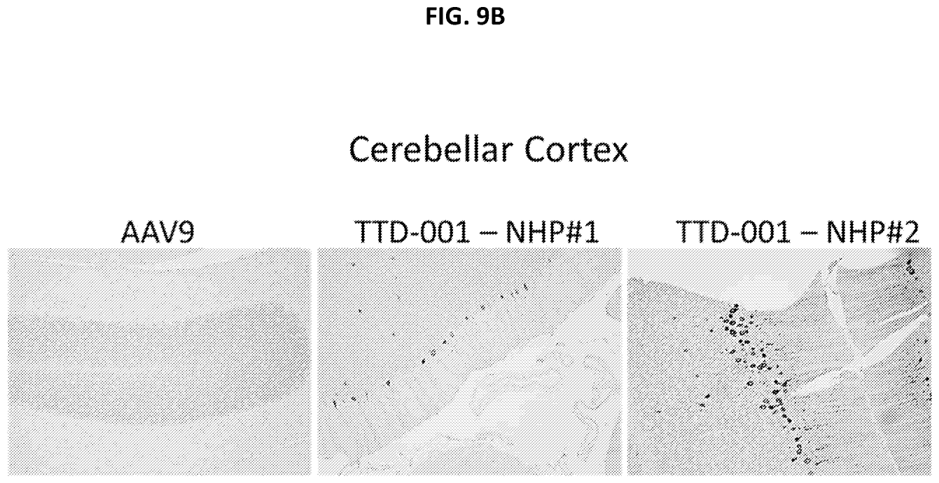

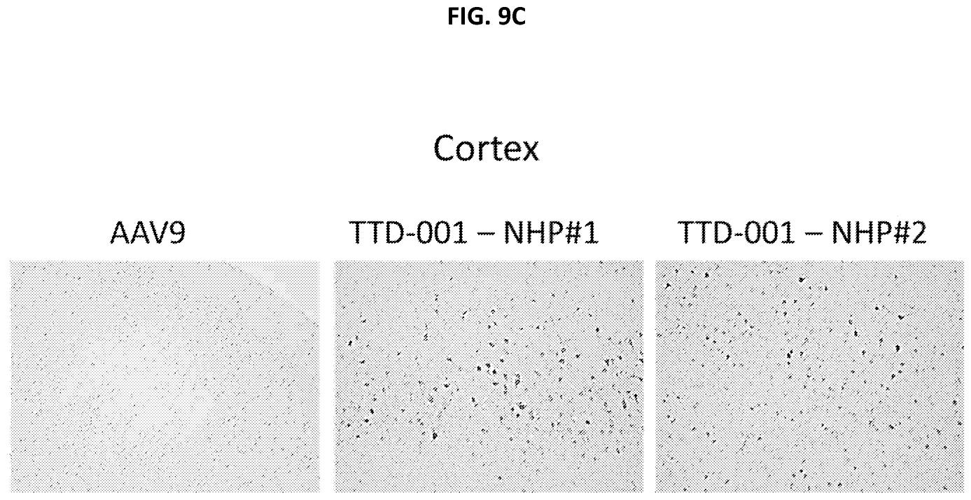

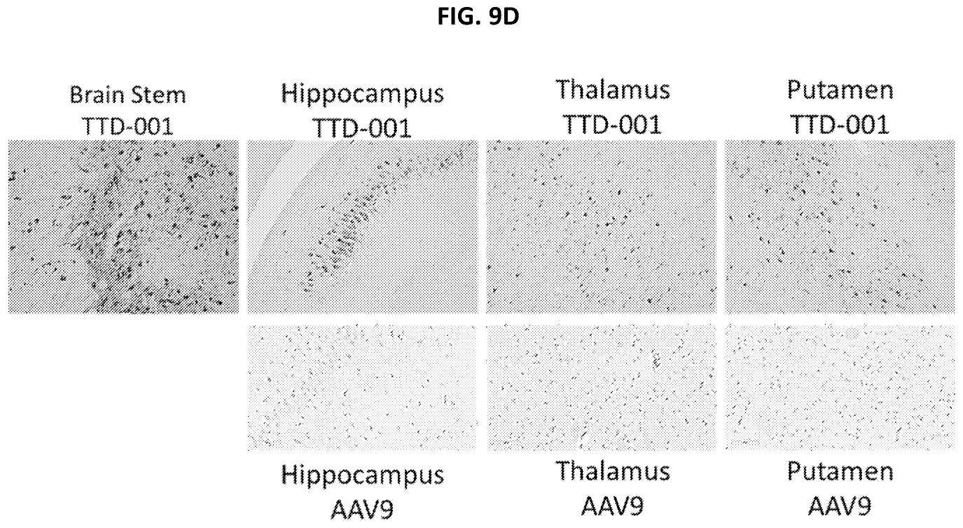

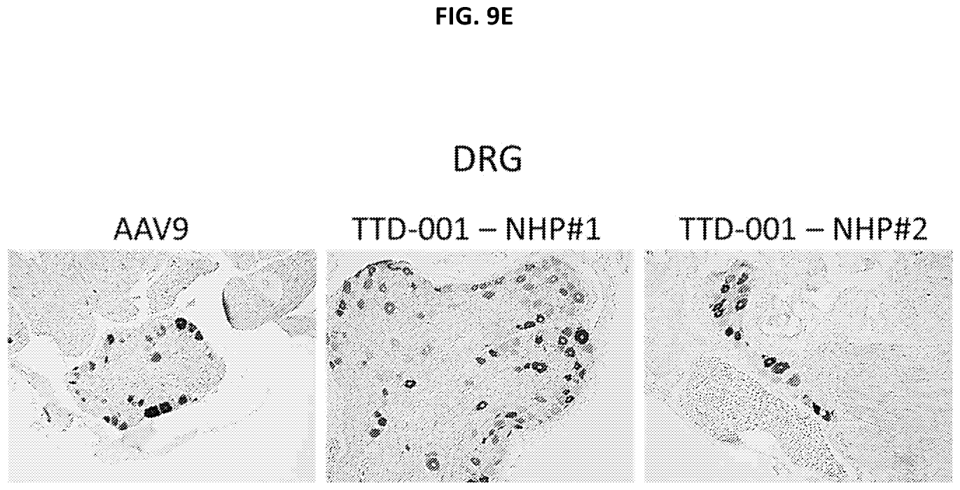

Total DNA was extracted from the same brain tissues as RNA, and biodistribution was measured by ddPCR using a Taqman set specific for the CMV promoter sequence. The RNAseP gene was used as a copy number reference. Vector genome (VG) per cell values were determined both by qPCR and ddPCR. Increased biodistribution was observed for the TTD-004 capsid in most brain regions, but surprisingly none of the other candidates showed a significant increase by comparison with AAV9. This apparent contradiction with the RNA quantification data could suggest that some capsids may present improved properties over AAV9 in post-attachment mechanisms rather than strict vector translocation in CNS parenchyma. Interestingly, DNA analysis confirmed the substantial detargeting of TTD-004 capsid from the DRG (FIG. 9A-9D of WO2021230987, the contents of which are hereby incorporated by reference in their entirety).

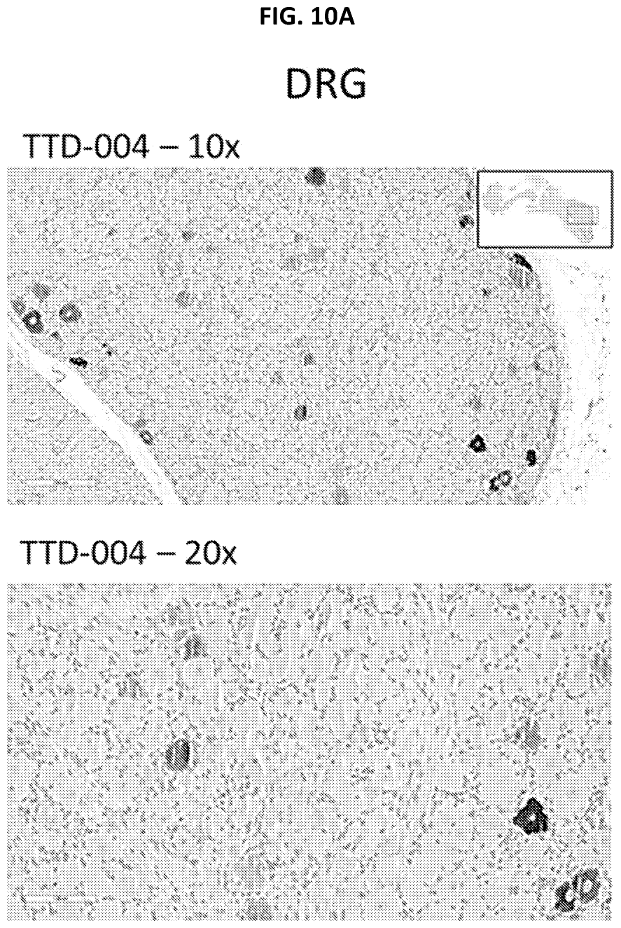

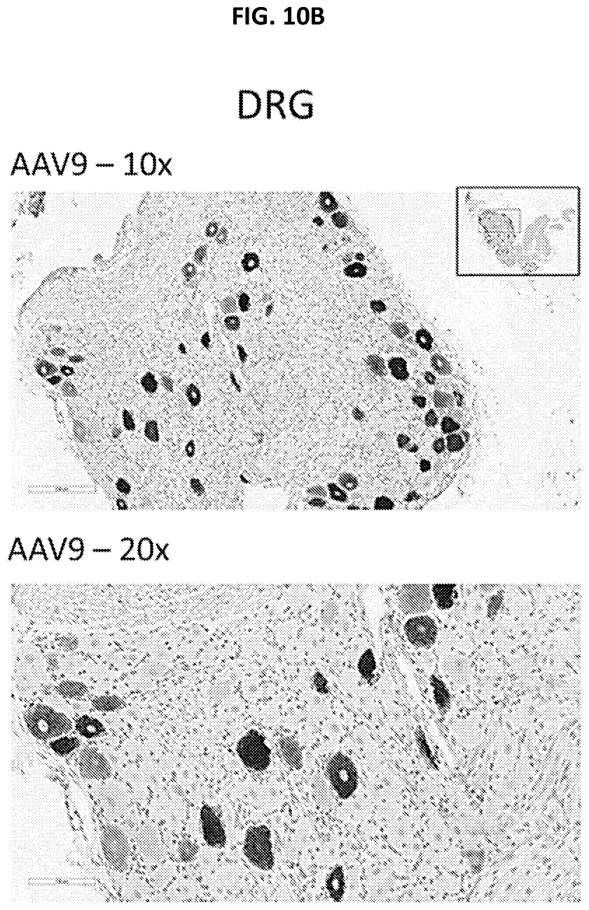

To further explore the behavior of capsid variant TTD-004, viral genome (VG) quantification was completed from tissues collected from heart atrium, heart ventricle, quadriceps muscle, liver (left and right) and diaphragm and compared to vector genome presence as delivered by AAV9 in the same tissues. The data are shown in FIGS. 10A and B of WO2021230987, the contents of which are hereby incorporated by reference in their entirety.

For TTD-003 and TTD-004 initial immunohistochemical analyses demonstrated the presence of payload-HA to a greater extent than seen with AAV9 delivery in cerebellar tissue, including in the dentate nucleus. Immunohistochemistry confirmed the de-targeting of the dorsal root ganglia for capsid variant TTD-004 as compared to TTD-003 and AAV9.

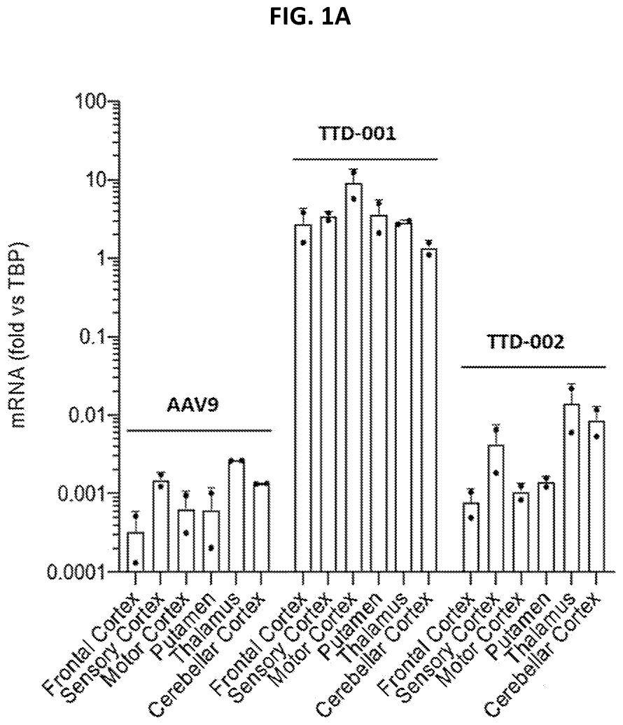

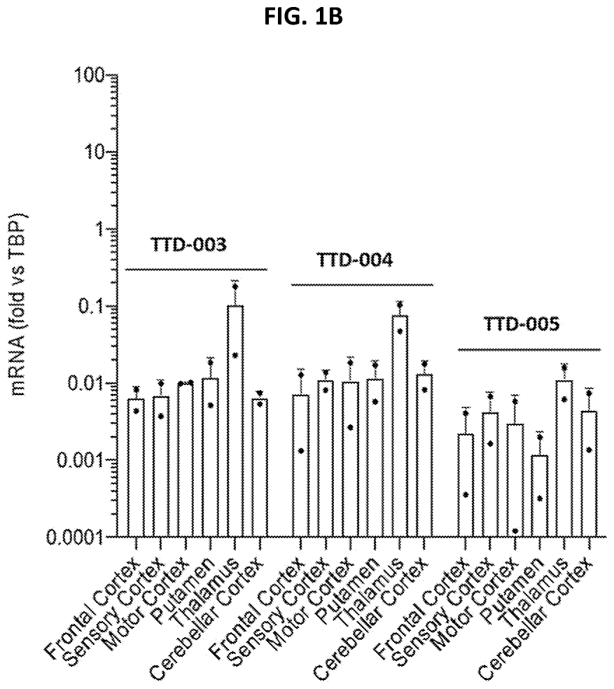

Data for each of the variants were compiled as an average mRNA (fold over TBP) or DNA (VG per cell) quantification per capsid variant per tissue as shown in Table 8 below and FIG. 1A-1B and FIG. 2A-2B, respectively

TABLE 8
Characterization of exemplary capsid variants

| Measure | Tissue | AAV9 | TTD-001 | TTD-002 | TTD-003 | TTD-004 | TTD-005 |
|---|---|---|---|---|---|---|---|
| mRNA | Frontal | 0.000325065 | 2.7232575 | 0.000768179 | 0.006268831 | 0.007076252 | 0.002204024 |
| | Cortex | | | | | | |
| mRNA | Sensory | 0.001486245 | 3.400055 | 0.00417739 | 0.006788644 | 0.010976612 | 0.004139604 |
| | Cortex | | | | | | |
| mRNA | Motor Cortex | 0.00063318 | 9.00819 | 0.001050247 | 0.009954825 | 0.010522399 | 0.002942249 |
| mRNA | Putamen | 0.000612759 | 3.557205 | 0.001395549 | 0.011832671 | 0.011476176 | 0.001150153 |
| mRNA | Thalamus | 0.002610992 | 2.863635 | 0.013937891 | 0.101411445 | 0.07565653 | 0.01100289 |
| mRNA | Cerebellar | 0.00133497 | 1.3439 | 0.008517779 | 0.006396677 | 0.012964181 | 0.004382119 |
| | Cortex | | | | | | |
| mRNA | Dentate | 0.001364954 | 0.963955 | — | — | — | — |
| | Nucleus | | | | | | |
| mRNA | Caudate | 0.000352281 | 1.3026 | — | 0.003259804 | 0.00634117 | — |
| mRNA | Hippocampus | 0.000311824 | 0.407015 | — | — | — | — |
| mRNA | SC-cervical | 0.012205449 | 11.877762 | 0.022004264 | 0.026994764 | 0.088316491 | 0.005773054 |
| mRNA | SC-Thoracic | 0.048833465 | 2.9974295 | 0.004360318 | 0.035118928 | 0.020543776 | 0.005629959 |
| mRNA | SC-Lumbar | 0.029887407 | 7.969603 | 0.056231995 | 0.016033388 | 0.047713563 | 0.026324154 |
| mRNA | DRG- | 0.74570895 | 9.274951 | 0.007897714 | 2.47872652 | 0.280868887 | 0.008122233 |
| | cervical | | | | | | |
| mRNA | DRG- | 0.5559061 | 5.22606 | 0.006456564 | 8.721845271 | 0.104701895 | — |
| | Thoracic | | | | | | |
| mRNA | DRG- | 1.089758 | 17.308436 | 0.008247771 | 2.271300217 | 0.426704698 | 0.119974244 |
| | Lumbar | | | | | | |
| mRNA | Lung | 0.004807149 | 0.000546842 | — | — | 0.013744781 | — |
| mRNA | Pancreas | — | — | — | — | — | — |
| mRNA | Colon | 0.017962678 | 0.005041385 | — | — | 0.183862903 | — |
| mRNA | Kidney | 0.043825993 | 0.006649157 | — | — | 0.041234576 | — |
| mRNA | Liver | 0.674478605 | 0.253188648 | — | — | 2.578654807 | — |
| mRNA | Adrenal | — | — | — | — | — | — |
| mRNA | Spleen | 0.014066875 | 0.000955981 | — | — | 0.013435626 | — |
| mRNA | Heart | 1.323389668 | 0.132477314 | — | — | 5.587929805 | — |
| mRNA | Quadriceps | 0.116623509 | — | — | — | 4.527799743 | — |
| mRNA | Diaphragm | 0.250001109 | — | — | — | 1.936435215 | — |
| DNA | Frontal | 0.07713 | 2.104843 | 0.10252 | 0.068367 | 0.380429 | 0.1257545 |
| | Cortex | | | | | | |
| DNA | Sensory | 0.093003 | 2.679886 | 0.07443 | 0.034016 | 0.2670975 | 0.132503 |
| | Cortex | | | | | | |
| DNA | Motor Cortex | 0.08796 | 4.3437625 | 0.0913085 | 0.094401 | 0.318999 | 0.1110695 |
| DNA | Putamen | 0.0581365 | 3.07904 | 0.12326 | 0.1497635 | 0.2731175 | 0.0715295 |
| DNA | Thalamus | 0.0524055 | 2.076863 | 0.0664225 | 0.090511 | 0.214999 | 0.086863 |
| DNA | Cerebellar | 0.014238 | 0.186361 | 0.0092915 | 0.009578 | 0.0356345 | 0.0128655 |
| | Cortex | | | | | | |
| DNA | Dentate | 0.025042 | 0.1861975 | 0.210238 | 0.041906 | 0.106107 | 0.055287 |
| | Nucleus | | | | | | |
| DNA | Caudate | 0.079294 | 3.9433175 | — | 0.0529005 | 0.2451035 | — |
| DNA | Hippocampus | 0.095436 | 1.760891 | 0.205433 | 0.368645 | 1.335324 | 0.432829 |
| DNA | SC-cervical | 0.0376 | 1.143863 | 0.061085 | 0.061535 | 0.07573 | 0.05885 |
| DNA | SC-Thoracic | 0.02692 | 0.933734 | 0.025955 | 0.05011 | 0.064915 | 0.0355 |
| DNA | SC-Lumbar | 0.03615 | 0.992728 | 0.019125 | 0.034175 | 0.085165 | 0.051475 |
| DNA | DRG- | 0.0765 | 0.14319 | 0.08196 | 0.13722 | 0.04115 | 0.071625 |
| | cervical | | | | | | |
| DNA | DRG- | 0.165865 | 0.172363 | 0.07202 | 0.133455 | 0.04444 | 0.03139 |
| | Thoracic | | | | | | |
| DNA | DRG- | 0.218725 | 0.385712 | 0.146115 | 0.153205 | 0.032875 | 0.12034 |
| | Lumbar | | | | | | |
| DNA | Lung | 1.085639916 | 3.72 | 0.958576278 | 0.700015423 | 1.22442329 | 0.919823152 |
| DNA | Pancreas | 0.256670617 | 20.535 | 0.320558325 | 0.240633195 | 0.067860607 | 0.004802583 |
| DNA | Colon | 0.053867646 | 3.405 | 1.179065405 | 0.348969617 | 0.116867365 | 0.015288464 |
| DNA | Kidney | 0.896656371 | 26.635 | 4.861362029 | 0.532746958 | 0.386522209 | 7.973793288 |
| DNA | Liver | 207.332334 | 217.64 | 111.910319 | 193.8349405 | 448.5980021 | 213.0317219 |
| DNA | Adrenal | 1.647725996 | 0.69 | 1.561129869 | 1.871878 | 1.269473156 | 0.847293047 |
| DNA | Spleen | 14.93815481 | 20.43565 | 51.70294001 | 22.79095714 | 6.514778227 | 45.91987284 |
| DNA | Heart | 2.012377817 | 14.49 | 0.757528914 | 1.780956673 | 3.814571986 | 0.44694144 |
| DNA | Quadriceps | 0.724278943 | 1.285 | 0.476250457 | 1.366015493 | 5.611203726 | 0.646197937 |
| DNA | Diaphragm | — | 1.06 | — | — | — | — |

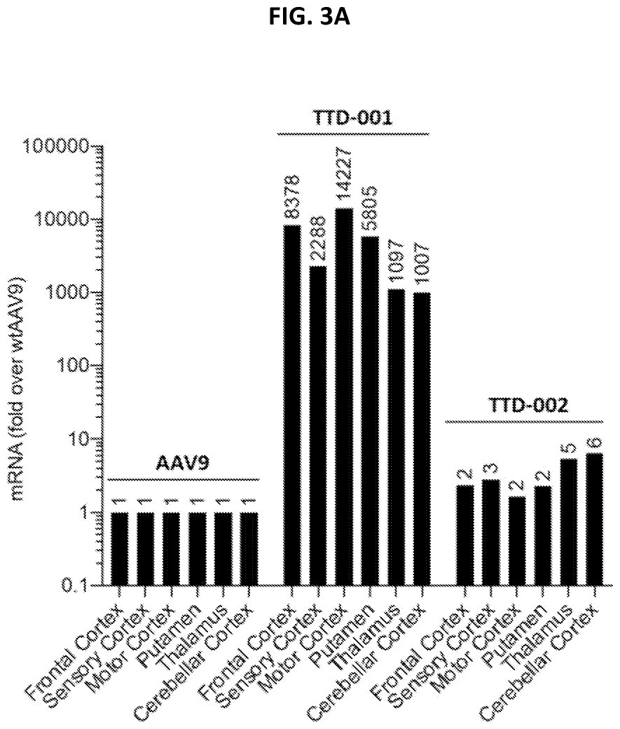

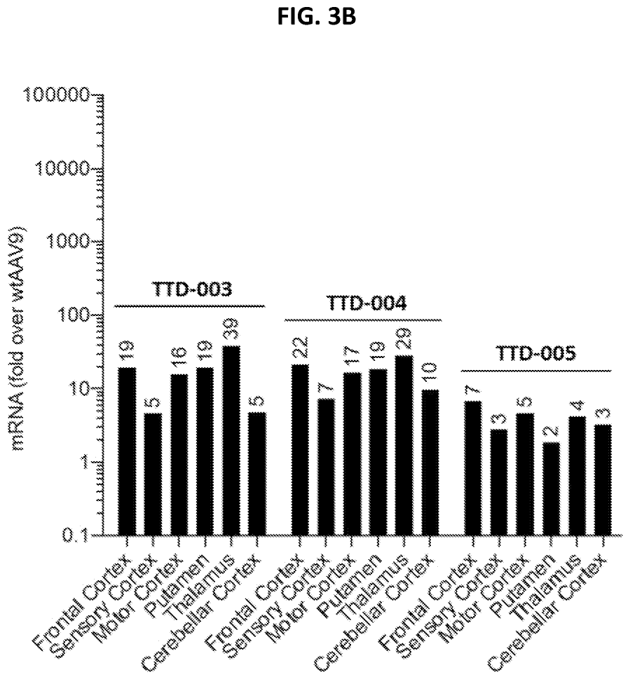

When calculated as fold over AAV9 the data were as shown in Table 9 below and FIGS. 3A and 3B and FIGS. 4A and 4B.

TABLE 9
Characterization of exemplary capsid variants

| Measure | Tissue | AAV9 | TTD-001 | TTD-002 | TTD-003 | TTD-004 | TTD-005 |
|---|---|---|---|---|---|---|---|
| mRNA | Frontal Cortex | 1.0 | 8378 | 2.4 | 19.3 | 21.8 | 6.8 |
| mRNA | Sensory Cortex | 1.0 | 2288 | 2.8 | 4.6 | 7.4 | 2.8 |
| mRNA | Motor Cortex | 1.0 | 14227 | 1.7 | 15.7 | 16.6 | 4.6 |
| mRNA | Putamen | 1.0 | 5805 | 2.3 | 19.3 | 18.7 | 1.9 |
| mRNA | Thalamus | 1.0 | 1097 | 5.3 | 38.8 | 29.0 | 4.2 |
| mRNA | Cerebellar | 1.0 | 1007 | 6.4 | 4.8 | 9.7 | 3.3 |
| | Cortex | | | | | | |
| mRNA | Dentate | 1.0 | 706 | — | — | — | — |
| | Nucleus | | | | | | |
| mRNA | Caudate | 1.0 | 3698 | — | — | — | — |
| mRNA | Hippocampus | 1.0 | 1305 | — | — | — | — |
| mRNA | SC-cervical | 1.0 | 973 | 1.8 | 2.2 | 7.2 | 0.5 |
| mRNA | SC-Thoracic | 1.0 | 61 | 0.1 | 0.7 | 0.4 | 0.1 |
| mRNA | SC-Lumbar | 1.0 | 267 | 1.9 | 0.5 | 1.6 | 0.9 |
| mRNA | DRG-cervical | 1.0 | 12 | 0.0 | 3.3 | 0.4 | 0.0 |
| mRNA | DRG-Thoracic | 1.0 | 9 | 0.0 | 15.7 | 0.2 | — |
| mRNA | DRG-Lumbar | 1.0 | 16 | 0.0 | 2.1 | 0.4 | 0.1 |
| mRNA | Lung | 1.0 | 0.11 | — | — | 2.9 | — |
| mRNA | Pancreas | — | — | — | — | — | — |
| mRNA | Colon | 1.0 | 0.28 | — | — | 10.2 | — |
| mRNA | Kidney | 1.0 | 0.15 | — | — | 0.9 | — |
| mRNA | Liver | 1.0 | 0.38 | — | — | 3.8 | — |
| mRNA | Adrenal | — | — | — | — | — | — |
| mRNA | Spleen | 1.0 | 0.07 | — | — | 1.0 | — |
| mRNA | Heart | 1.0 | 0.10 | — | — | 4.2 | — |
| mRNA | Quadriceps | 1.0 | — | — | — | 38.8 | — |
| mRNA | Diaphragm | 1.0 | — | — | — | 7.7 | — |
| DNA | Frontal Cortex | 1.0 | 27.29 | 1.3 | 0.9 | 4.9 | 1.6 |
| DNA | Sensory Cortex | 1.0 | 28.82 | 0.8 | 0.4 | 2.9 | 1.4 |
| DNA | Motor Cortex | 1.0 | 49.38 | 1.0 | 1.1 | 3.6 | 1.3 |
| DNA | Putamen | 1.0 | 52.96 | 2.1 | 2.6 | 4.7 | 1.2 |
| DNA | Thalamus | 1.0 | 39.63 | 1.3 | 1.7 | 4.1 | 1.7 |
| DNA | Cerebellar | 1.0 | 13.09 | 0.7 | 0.7 | 2.5 | 0.9 |
| | Cortex | | | | | | |
| DNA | Dentate | 1.0 | 7.44 | 8.4 | 1.7 | 4.2 | 2.2 |
| | Nucleus | | | | | | |
| DNA | Caudate | 1.0 | 49.73 | — | 0.7 | 3.1 | — |
| DNA | Hippocampus | 1.0 | 18.45 | 2.2 | 3.9 | 14.0 | 4.5 |
| DNA | SC-cervical | 1.0 | 30.42 | 1.6 | 1.6 | 2.0 | 1.6 |
| DNA | SC-Thoracic | 1.0 | 34.69 | 1.0 | 1.9 | 2.4 | 1.3 |
| DNA | SC-Lumbar | 1.0 | 27.46 | 0.5 | 0.9 | 2.4 | 1.4 |
| DNA | DRG-cervical | 1.0 | 1.87 | 1.1 | 1.8 | 0.5 | 0.9 |
| DNA | DRG-Thoracic | 1.0 | 1.04 | 0.4 | 0.8 | 0.3 | 0.2 |
| DNA | DRG-Lumbar | 1.0 | 1.76 | 0.7 | 0.7 | 0.2 | 0.6 |
| DNA | Lung | 1.0 | 3.43 | 0.9 | 0.6 | 1.1 | 0.8 |
| DNA | Pancreas | 1.0 | 80.01 | 1.2 | 0.9 | 0.3 | 0.0 |
| DNA | Colon | 1.0 | 63.21 | 21.9 | 6.5 | 2.2 | 0.3 |
| DNA | Kidney | 1.0 | 29.70 | 5.4 | 0.6 | 0.4 | 8.9 |
| DNA | Liver | 1.0 | 1.05 | 0.5 | 0.9 | 2.2 | 1.0 |
| DNA | Adrenal | 1.0 | 0.42 | 0.9 | 1.1 | 0.8 | 0.5 |
| DNA | Spleen | 1.0 | 1.37 | 3.5 | 1.5 | 0.4 | 3.1 |
| DNA | Heart | 1.0 | 7.20 | 0.4 | 0.9 | 1.9 | 0.2 |
| DNA | Quadriceps | 1.0 | 1.77 | 0.7 | 1.9 | 7.7 | 0.9 |
| DNA | Diaphragm | — | — | — | — | — | — |

Capsid variant TTD-001 showed greater than 5,000 fold increase in payload-HA levels delivered to the brain as compared to AAV9 and measured by qRT-PCR and normalized to TBP. In all CNS tissues measured, TTD-001 showed dramatically enhanced delivery of payload-HA as compared to AAV9.

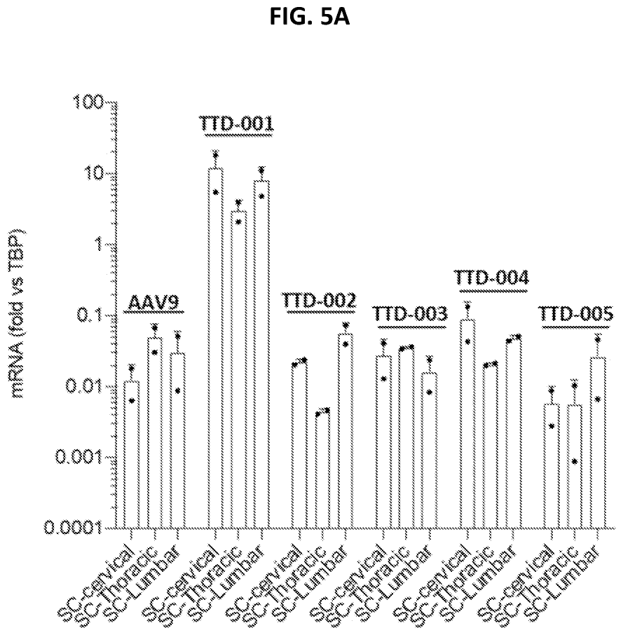

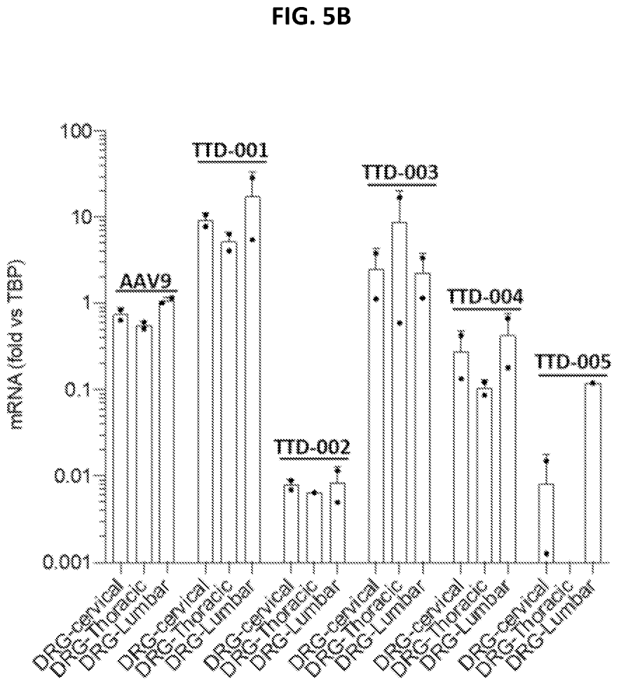

Graphical representations of the spinal cord and dorsal root ganglia measurements outlined in Tables 8 and 9 are shown in FIGS. 5A-5B, FIGS. 6A-6B, FIGS. 7A-7B, and FIGS. 8A-8B.

Immunohistochemistry of fixed brain tissues revealed dramatic transduction in both NHP tested by TTD-001 of the dentate nucleus, cerebellar cortex, cerebral cortex, brain stem, hippocampus, thalamus and putamen. AAV9 transduction of the dentate nucleus, cerebellar cortex, cerebral cortex, hippocampus, thalamus and putamen appeared negligible in comparison. TTD-001 therefore demonstrated broad and robust expression and distribution in the brain following intravenous administration in NHPs. In the dorsal root ganglia, both TTD-001 and AAV9 showed similar IHC patterns. Images of these stainings are shown in FIGS. 9A-9E.

Immunohistochemical support for the DRG de-targeting nature of capsid variant TTD-004 (as noted above) is shown in FIGS. 10A-10B.

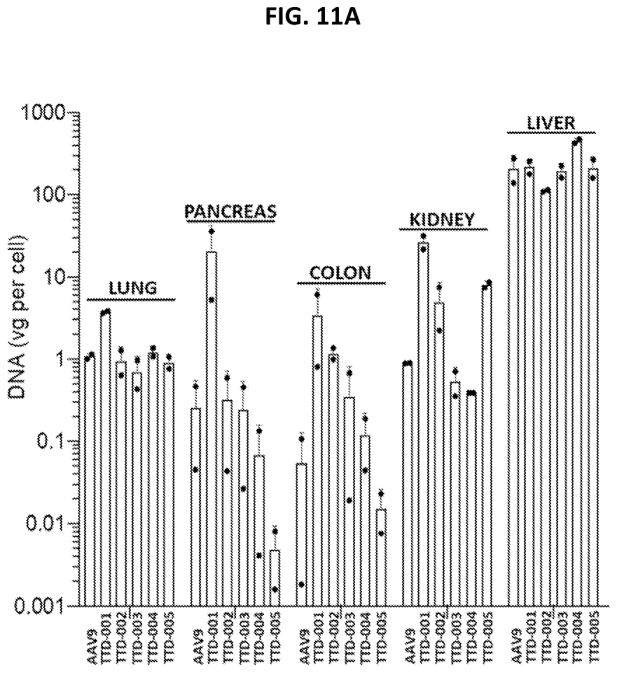

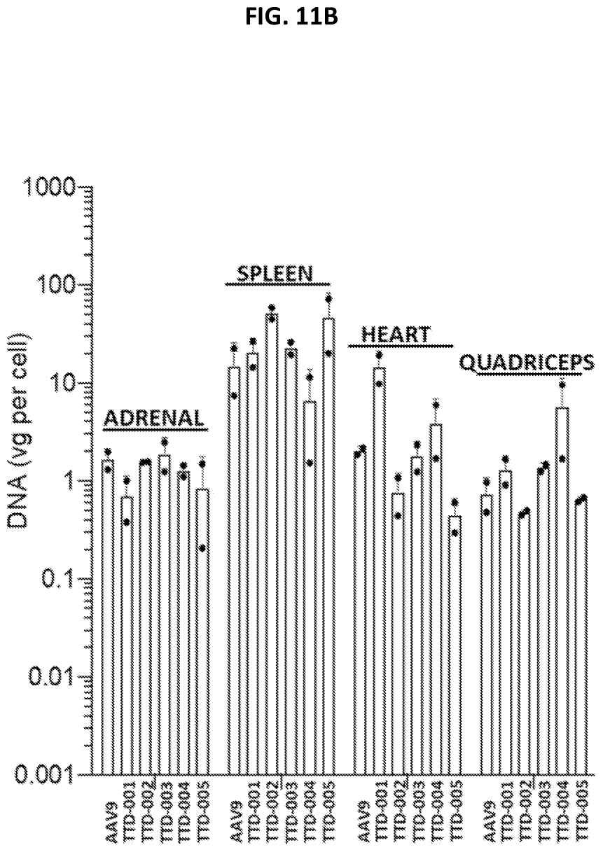

Graphical representations of the biodistribution of viral genomes delivered by variant capsids or AAV9 to peripheral tissues are shown in FIG. 11A-11B.

### Example 6. Individual Capsid Characterization in the Heart

This Example characterized the transduction level and the spatial distribution the TTD-001 (SEQ ID NO: 3623 and 3636, comprising SEQ ID NO: 1725 or 3648) and TTD-004 (SEQ ID NO: 3627 and 3639, comprising SEQ ID NO: 1760 or 3651) capsid variants in the heart muscle.

AAV particles were generated with each of a TTD-001 and TTD-004 capsid variant or a wild-type AAV9 capsid polypeptide control, encapsulating a transgene encoding a payload fused to an HA tag (payload-HA) and driven by a full-length CMV/chicken beta actin promoter. The AAV particles comprising the TTD-001 or TTD-004 capsid variant or the wild-type AAV9 capsid control, were administered intravenously to 2 female NHPs at a dose of 2e13 VG/kg. At day 14 post-administration of the AAV particles, the heart tissue was collected, fixed, and paraffin embedded for immunohistochemical staining. An anti-HA antibody (Cell Signal Technology) was used for staining the heart tissue for visualization of the transduction and distribution of the AAV capsid variants investigated. Both left and right heart ventricle samples were collected and analyzed.

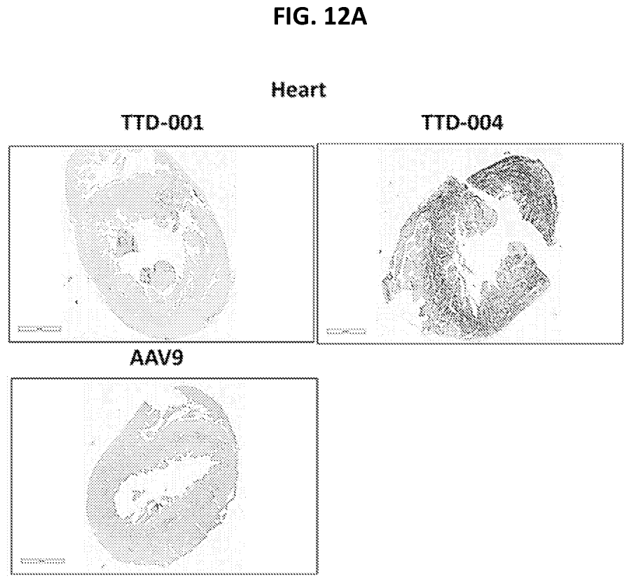

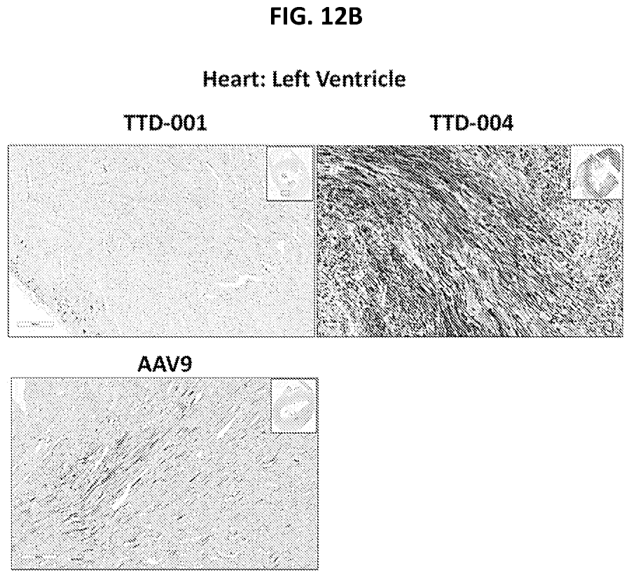

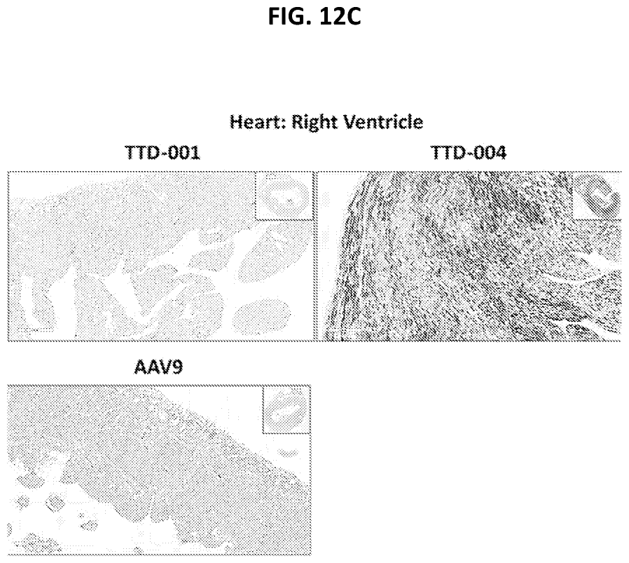

As shown in FIG. 12A-12C, immunohistochemistry of the fixed heart tissue and cardiomyocytes, demonstrated transduction by both TTD-001 and TTD-004, in the left ventricle (FIG. 12B) and right ventricle (FIG. 12C). However, TTD-004 led to the greatest transduction in the left and right ventricle regions of the heart (FIG. 12B-12C), as compared to TTD-001 and the wild-type AAV9 control, as evidenced by increased IHC staining. TTD-001 and the wild-type AAV9 control demonstrated similar levels of transduction in both regions of the heart, as evidenced by similar IHC staining patterns. These data demonstrate that both the TTD-001 and TTD-004 capsid variants can be utilized to transduce and/or deliver a payload, e.g., a payload described herein, to a heart muscle.

### Example 7. Maturation of TTD-001 Capsid in NHPs

This Example describes maturation of the TTD-001 (SEQ ID NO: 3623 (DNA) and 3636 (amino acid), comprising SEQ ID NO: 1725 or 3648) capsid variant to further enhance its transduction and biodistribution in the central nervous system and evolve the AAV capsid variants further. Two approaches were used to mature the TTD-001 capsid sequence in order to randomize and mutate within and around the peptide insert comprised within loop VIII of the capsid variant. In the first maturation approach, sets of three contiguous amino acids were randomized across the mutagenesis region in the TTD-001 sequence, which spanned from position 587 to position 602, numbered according to SEQ ID NO: 3636. In the second maturation approach, mutagenic primers were used to introduce point mutations at a low frequency, scattered across the mutagenesis region in the TTD-001 sequences ranging from position 587 to position 602, numbered according to SEQ ID NO: 3636. AAV capsid variants arising from each maturation approach for TTD-001 were pooled together, for subsequent testing and characterization in NHPs (Macaca fascicularis and Callithrix jacchus).

The library of pooled matured AAV capsid variants generated from TTD-001 matured AAV capsid variant were injected into two cynomolgus macaques (Macaca fascicularis), two marmosets (Callithrix jacchus). After a period in life, the brains of the NHPs were isolated and RNA was extracted from three samples per NHP. Following RNA recovery and RT-PCR amplification, a systematic NGS enrichment analysis was performed to calculate the fold enrichment ratio relative to the corresponding TTD-001 control, and the peptides comprised within the variants were identified. The coefficient of variance (CV) was calculated for each peptide across the six samples, and those that had a CV value <1 were identified, as these were the peptides that were reliably detected in 5/6 or 6/6 of the brain samples isolated from the two NHPs. The average number of reads for each peptide across the samples investigated was also quantified. These TTD-001 matured capsid variants and their peptide sequences are provided in Table 10 (cynomolgus macaques (Macaca fascicularis)) and Table 20 (marmosets (Callithrix jacchus)).

As shown in Table 10, approximately 338 TTD-001 matured capsid variants demonstrated increased expression relative to the non-matured TTD-001 control, and several variants demonstrated greater than a two-fold enrichment relative to the non-matured TTD-001 control, in cynomolgus macaques (Macaca fascicularis). Also, across the peptides comprised within the TTD-001 matured capsid variants with the greatest fold-enrichment relative to the non-matured TTD-001 capsid in the brains of cynomolgus macaques, it was observed that the modifications in the variant sequences appeared in the C-terminal portion, specifically at residues corresponding to positions 593-595 of a reference sequence numbered according to the amino acid sequence of SEQ ID NO: 138. Additionally, 378 of the top peptides in Table 10 had an average read value of 1 or greater per sample, demonstrating that more functional capsid material was recovered, which could be indicative of less aggregation.

As shown in Table 20, many TTD-001 matured capsid variants demonstrated increased expression relative to both the AAV9 and the non-matured TTD-001 controls in the brains of marmosets. Approximately, 967 TTD-001 matured variants demonstrated increased expression relative to the non-matured TTD-001 control in the brain of marmosets, with 296 variants showing at least a 10-fold enrichment or greater relative to the non-matured TTD-001 control. Approximately, 850 TTD-001 matured variants demonstrated increased expression relative to AAV9 in the brain of marmosets, with 222 variants showing at least a 10-fold enrichment or greater relative to AAV9. With respect to those TTD-001 matured variants that demonstrated an increased expression in marmosets, it was observed that the majority comprised an amino acid other than Q at position 604 (e.g., Q604) numbered according to SEQ ID NO: 5, 8, or 3636 or at position 597 (Q597) numbered according to SEQ ID NO: 138 (e.g., an E, H, K, or P), such that they comprised the triplet “VEN,” “VHN,” “VKN,” or “VPN” at their C-terminus (corresponding to positions 596-598 of SEQ ID: 138 or positions 603-605 of SEQ ID NO: 5, 8, and 3636). Many of these TTD-001 matured variants also demonstrated an increased expression in the brain of cynomolgus macaques relative to AAV9 (Table 20), including the TTD-001 matured capsid variant comprising the sequence PLNGAVHLYAQAQLSPVKN (SEQ ID NO: 566) and the TTD-001 matured capsid variant comprising the sequence PLNGAVHLYAQAQTGWVPN (SEQ ID NO: 314).

The fold-change in expression relative to AAV9 and TTD-001 was also calculated in the DRG, heart, muscle (quadriceps), and liver for the TTD-001 matured variants in cynomolgus macaques. The fold-change in the DRG is shown in Table 20, with several variants showing decreased or comparable expression in the DRG relative to AAV9. These variants also demonstrated comparable or lower expression relative to AAV9 in the heart, muscle, and liver.

Taken together, these data demonstrate that following two maturation approaches, matured TTD-001 capsid variants with loop VIII modifications were generated with significantly enhanced CNS tropism in NHPs (cynomolgus macaques (Macaca fascicularis) and marmosets (Callithrix jacchus)), compared to the corresponding non-matured TTD-001 capsid variant, which already exhibited a significant fold enrichment over AAV9 in the NHP brain.

TABLE 10
NGS fold-enrichment of TTD-001 matured AAV capsid variants in the brain of NHPs
(Macaca fascicularis)

| | | | Fold | | | | | Fold | |
|---|---|---|---|---|---|---|---|---|---|
| | SEQ | | enrichment | | | SEQ | | enrichment | |
| Peptide | ID | | over TTD- | Avg. | Peptide | ID | | over TTD- | Avg. |
| Sequence | NO: | CV | 001 | Reads | Sequence | NO: | CV | 001 | Reads |
| QLNGAVHLYA | 139 | 0.206 | 5.617 | 0.09 | QLNGYVHLYA | 1138 | 0.513 | 0.626 | 0.09 |
| QAQLSPVQN | | | | | QAQTGWVQN | | | | |
| PLDGAVHLYA | 140 | 0.829 | 5.133 | 4.02 | QLNGAVHLYA | 639 | 0.845 | 0.626 | 0.09 |
| QPQTGWVQN | | | | | QAQTTGVQN | | | | |
| QLNGAVHLYA | 141 | 0.770 | 4.088 | 0.09 | PLNGAVHLYA | 640 | 0.602 | 0.625 | 39.96 |
| QAQTMSVQN | | | | | QAQTGFVQN | | | | |
| PLDSSVHLYA | 142 | 0.882 | 3.891 | 1.4 | PLNGAVHLYA | 641 | 0.536 | 0.625 | 42.62 |
| QAQTGWVQN | | | | | QAQSALVQN | | | | |
| PLNGAVHLYA | 143 | 0.879 | 3.879 | 5.47 | ALNGAVHLYA | 642 | 0.278 | 0.624 | 0.75 |
| QAQTTKVQN | | | | | QAQTGWDQN | | | | |
| PLDGAVHLYA | 144 | 0.586 | 3.835 | 11.08 | QLNGAVHLYA | 643 | 0.636 | 0.623 | 0.09 |
| QAQTGSVQN | | | | | QAQISGVQN | | | | |
| ALNGAVHLYA | 145 | 0.865 | 3.765 | 0.09 | QLNGAVHLYA | 644 | 0.520 | 0.623 | 0.14 |
| QAQTTSVQN | | | | | QAQRGSVQN | | | | |
| PLNGSVHLYA | 146 | 0.509 | 3.708 | 0.09 | PLNGAVHLYA | 645 | 0.270 | 0.622 | 0.19 |
| QAQTMSVQN | | | | | QAQTGWVKT | | | | |
| QLNGAVHLYA | 147 | 0.310 | 3.638 | 0.14 | PLNGAVHLYA | 646 | 0.554 | 0.619 | 27.62 |
| QAQTSPVQN | | | | | QAQSQLVQN | | | | |
| PLNGAVHLYA | 148 | 0.547 | 3.634 | 5.61 | PLNGSVHLYA | 647 | 0.202 | 0.617 | 0.33 |
| QAQTMKVQN | | | | | QAQTGWGQN | | | | |
| PLNGAVHLYA | 149 | 0.397 | 3.550 | 21.87 | QLNGAVHLYA | 648 | 0.632 | 0.616 | 0.14 |
| QAQVAQVQN | | | | | QAQTGWVPN | | | | |
| PLDGAVHLYA | 150 | 0.771 | 3.509 | 2.29 | PLNGAVHLYA | 649 | 0.305 | 0.615 | 0.33 |
| QAQTGGVQN | | | | | KAQTGWVKN | | | | |
| PLNGAVHLYA | 151 | 0.344 | 3.373 | 0.09 | PLNGAVHLYA | 650 | 0.164 | 0.615 | 0.09 |
| QAQTAWDQN | | | | | HAQTGWGQN | | | | |
| PLNGSVHLYA | 152 | 0.190 | 3.335 | 0.23 | ALNGAVHLYA | 651 | 0.330 | 0.613 | 0.61 |
| QAQTGWDQN | | | | | QAQTGWVQH | | | | |
| PLNGAVHLYA | 153 | 0.235 | 3.287 | 0.14 | PLNGSVHLYA | 652 | 0.585 | 0.612 | 0.14 |
| QAQTGSVQH | | | | | QAQIASVQN | | | | |
| PLNGAVHLYA | 154 | 0.564 | 3.259 | 4.25 | ALNGAVHLYA | 653 | 0.416 | 0.611 | 0.14 |
| QAQVKQVQN | | | | | QAQTAMVQN | | | | |
| PLNGAVHLYA | 155 | 0.763 | 3.239 | 24.54 | TLNGAVHLYA | 654 | 0.316 | 0.610 | 0.09 |
| QAQSAPVQN | | | | | QAQTMAVQN | | | | |
| PLNGAVHLYA | 156 | 0.591 | 3.156 | 6.73 | PHNGAVHLYA | 655 | 0.476 | 0.609 | 0.09 |
| QAQLSKVQN | | | | | QAQVSSVQN | | | | |
| PLNGAVHLYA | 157 | 0.587 | 3.107 | 31.03 | PLNGAVQLYA | 656 | 0.693 | 0.606 | 0.09 |
| QAQLAPVQN | | | | | QAQTAPVQN | | | | |
| PLNGAVHLYA | 158 | 0.416 | 3.061 | 15.33 | PLNGSVHLYA | 657 | 0.282 | 0.605 | 0.09 |
| QAQLAQVQN | | | | | QAQKGSVQN | | | | |
| QLNGAVHLYA | 159 | 0.295 | 3.051 | 0.09 | PLNGAVHLYA | 658 | 0.574 | 0.605 | 39.3 |
| QAQVASVQN | | | | | QAQLSAVQN | | | | |
| PLNGAVHLYA | 160 | 0.877 | 2.998 | 8.97 | PINGAVHLYA | 659 | 0.489 | 0.605 | 0.09 |
| QAQTAKVQN | | | | | QAQVSAVQN | | | | |
| PLNGAVHLYA | 161 | 0.678 | 2.971 | 3.27 | PLNGSVHLYA | 660 | 0.514 | 0.604 | 43.37 |
| QAQSAKVQN | | | | | QAQTGLVQN | | | | |
| PLNGAVHLYA | 162 | 0.095 | 2.962 | 0.09 | PLNGAVHIYA | 661 | 0.223 | 0.604 | 0.14 |
| QAQTGCFQN | | | | | QAQTGWFQN | | | | |
| PLNGAVHLYA | 163 | 0.705 | 2.958 | 5.51 | SLNGAVHLYA | 662 | 0.293 | 0.603 | 0.09 |
| QAQTQKVQN | | | | | QAQIASVQN | | | | |
| PLNGSVHLYA | 164 | 0.529 | 2.873 | 0.09 | PLNGAVHLYA | 663 | 0.220 | 0.601 | 0.14 |
| QAQTTSVQN | | | | | QEQTVSVQN | | | | |
| PLNGGVHLYA | 165 | 0.820 | 2.858 | 4.25 | PLNGAVHLYA | 664 | 0.737 | 0.601 | 0.09 |
| QAQTGRVQN | | | | | QAQTMQVQT | | | | |
| PLNGAVHLYA | 166 | 0.410 | 2.854 | 30.94 | HLNGAVHLYA | 665 | 0.338 | 0.600 | 56.78 |
| QAQTVAVQN | | | | | QAQTGWVQN | | | | |
| ALNGAVHLYA | 167 | 0.542 | 2.851 | 0.09 | PLNGAVHLYA | 666 | 0.400 | 0.600 | 24.02 |
| QAQSSPVQN | | | | | QAQTGQVQN | | | | |
| PLNGAVHLYA | 168 | 0.313 | 2.820 | 63.28 | QLNGAVHLYA | 667 | 0.596 | 0.599 | 0.19 |
| QAQLSPVQN | | | | | QAQTGWVQD | | | | |
| QLNGAVHLYA | 169 | 0.846 | 2.769 | 0.14 | PLNGAVHLYD | 668 | 0.526 | 0.599 | 0.09 |
| QAQTTSVQN | | | | | QAQVAQVQN | | | | |
| PLNGAVHLYA | 170 | 0,334 | 2.719 | 18.37 | PLNGAVHLYA | 669 | 0.515 | 0.598 | 0.19 |
| QAQTTQVQN | | | | | QAQTSPVKN | | | | |
| PLNGAVHLYA | 171 | 0.523 | 2.707 | 25.84 | PLNGAIHLYA | 670 | 0.383 | 0.595 | 0.09 |
| QAQTAQVQN | | | | | QAQTSPVQN | | | | |
| QLNGAVHLYA | 172 | 0.195 | 2.682 | 0.09 | PLNGSVHLYA | 671 | 0.578 | 0.595 | 0.19 |
| QAQTVAVQN | | | | | QAQTAMVQN | | | | |
| PLNGAVHLYA | 173 | 0.682 | 2.652 | 3.27 | PLNGAVHLYA | 672 | 0.370 | 0.594 | 0.09 |
| QAQRIAVQN | | | | | QAQLSQVQT | | | | |
| PLNGAVHLYA | 174 | 0.340 | 2.634 | 17.34 | PLNGAVHLYA | 673 | 0.310 | 0.594 | 0.19 |
| QAQRASVQN | | | | | QAQTGSVLN | | | | |
| PLNGAVHLYA | 175 | 0.400 | 2.620 | 45.19 | PLNGAIHLYA | 674 | 0.428 | 0.593 | 0.09 |
| QAQTTPVQN | | | | | QAQTASVQN | | | | |
| QLNGAVHLYA | 176 | 0.474 | 2.613 | 0.09 | PLNGAVHLYA | 675 | 0.785 | 0.592 | 6.68 |
| QAQLASVQN | | | | | QAQTNGVQN | | | | |
| PLNGAVHLYA | 177 | 0.528 | 2.574 | 39.86 | PLNGSVHLYA | 676 | 0.334 | 0.591 | 0.14 |
| QAQLTPVQN | | | | | QAQTGWVQY | | | | |
| PLNGAVHLYA | 178 | 0.519 | 2.572 | 26.5 | PLNGALHLYA | 677 | 0.463 | 0.590 | 0.09 |
| QAQSTPVQN | | | | | QAQTSPVQN | | | | |
| PLNGAVHLYA | 179 | 0.335 | 2.562 | 72.81 | ALNGAVHLYA | 678 | 0.399 | 0.589 | 1.59 |
| QAQTSPVQN | | | | | QAQTGWVQT | | | | |
| QLNGAVHLYA | 180 | 0.428 | 2.528 | 0.09 | PLNGAVHLYA | 679 | 0.193 | 0.586 | 0.47 |
| QAQVSQVQN | | | | | QAQTGWVKK | | | | |
| PLNGAVHLYA | 181 | 0.625 | 2.523 | 19.96 | QLNGAVHLYA | 680 | 0.630 | 0.584 | 1450 |
| QAQTMQVQN | | | | | QAQTGWVQN | | | | |
| PLNGAVHLYA | 182 | 0.458 | 2.476 | 8.32 | PLNGDVHLYA | 681 | 0.630 | 0.580 | 0.09 |
| QAQTSKVQN | | | | | QAQTVAVQN | | | | |
| SLNGAVHLYA | 183 | 0.282 | 2.417 | 0.09 | PLNGGVHLYA | 682 | 0.501 | 0.580 | 0.47 |
| QAQVSPVQN | | | | | QAQPGWVQN | | | | |
| PLNGAVHLYA | 184 | 0.259 | 2.411 | 34.72 | PLNGAVHLYA | 683 | 0.897 | 0.579 | 7.66 |
| QAQVSQVQN | | | | | QAQAAAVQN | | | | |
| PLNGAVHLYA | 185 | 0.721 | 2.404 | 63.42 | PLNGAVHLYA | 684 | 0.618 | 0.579 | 19.72 |
| QAQVSPVQN | | | | | QAQSAVVQN | | | | |
| PLNGAVHLYA | 186 | 0.586 | 2.389 | 15.56 | TLNGAVHLYA | 685 | 0.646 | 0.578 | 0.09 |
| QAQTVQVQN | | | | | QAQRSSVQN | | | | |
| PLNGAVHLYA | 187 | 0.476 | 2.367 | 13.65 | PLNGAVHLYA | 686 | 0.214 | 0.578 | 0.14 |
| QAQVTAVQN | | | | | QAQPGWVQH | | | | |
| PLNGAVHLYA | 188 | 0.715 | 2.353 | 8.74 | PLDGAVHLYA | 687 | 0.214 | 0.577 | 0.09 |
| QAQRQPVQN | | | | | QAQTLSVQN | | | | |
| PLNGNVHLYA | 189 | 0.732 | 2.346 | 2.38 | PLNGAVHLYA | 688 | 0.310 | 0.575 | 0.19 |
| QAQTGGVQN | | | | | QAQTASDQN | | | | |
| PLDGAVHLYA | 190 | 0.697 | 2.339 | 7.06 | PLNGAVHLYA | 689 | 0.633 | 0.575 | 53.51 |
| QAQTAWVQN | | | | | QAQLSGVQN | | | | |
| PLNGAVHLYA | 191 | 0.785 | 2.331 | 9.91 | TLNGAVHLYA | 690 | 0.463 | 0.572 | 0.14 |
| QAQISGVQN | | | | | QAQTVAVQN | | | | |
| PLNGAVHLYA | 192 | 0.794 | 2.324 | 6.22 | PLNGAVHLYA | 691 | 0.293 | 0.572 | 0.09 |
| QAQVRPVQN | | | | | QEQVASVQN | | | | |
| PLNGAVHLYA | 193 | 0.722 | 2.299 | 20.47 | PVNGAVHLYA | 692 | 0.873 | 0.572 | 0.09 |
| QAQLGPVQN | | | | | QAQTGWAQN | | | | |
| PLNGAVHLYA | 194 | 0.404 | 2.290 | 4.91 | PLNGAVHLYA | 693 | 0.567 | 0.570 | 14.07 |
| QAQTNQVQN | | | | | QAQSSRVQN | | | | |
| PLNGAVHLYA | 195 | 0.291 | 2.240 | 14.39 | PLNGSVHLYA | 694 | 0.551 | 0.569 | 0.19 |
| QAQVQQVQN | | | | | QAQLGSVQN | | | | |
| PLNGAVHLYA | 196 | 0.520 | 2.238 | 9.21 | PLNGAVHLYA | 695 | 0.565 | 0.568 | 1.22 |
| QAQVANVQN | | | | | QAQSPPVQN | | | | |
| PLNGAVHLYA | 197 | 0.664 | 2.200 | 10.75 | PLNGAVHHYA | 696 | 0.605 | 0.567 | 0.14 |
| QAQAAPVQN | | | | | QAQTTSVQN | | | | |
| QLNGAVHLYA | 198 | 0.388 | 2.199 | 0.09 | PLNGAVHLYD | 697 | 0.832 | 0.567 | 0.09 |
| QAQVAQVQN | | | | | QAQSTPVQN | | | | |
| PLNGAVHLYA | 199 | 0.487 | 2.195 | 21.59 | QLNGAVHLYA | 698 | 0.529 | 0.567 | 0.37 |
| QAQRSTVQN | | | | | QAQTGWVRN | | | | |
| PLNGAVHLYA | 200 | 0.334 | 2.190 | 29.72 | PLNGAVHLYA | 699 | 0.597 | 0.563 | 0.09 |
| QAQTMAVQN | | | | | QAQVQPVQT | | | | |
| PLNGAVHLYA | 201 | 0.838 | 2.182 | 7.1 | PLNGAVHLYA | 700 | 0.416 | 0.562 | 0.14 |
| QAQIQPVQN | | | | | QAQTSSVKN | | | | |
| PLNGAVHLYA | 202 | 0.567 | 2.181 | 15.52 | PLNGALHLYA | 701 | 0.301 | 0.560 | 0.09 |
| QAQIASVQN | | | | | QAQTASVQN | | | | |
| PLNGAVHLYA | 203 | 0.172 | 2.176 | 76.22 | PLNGAVHHYA | 702 | 0.731 | 0.558 | 0.09 |
| QAQTVSVQN | | | | | QAQSAPVQN | | | | |
| PLNGAVHLYA | 204 | 0.585 | 2.156 | 12.43 | PLNGSVHLYA | 703 | 0.169 | 0.556 | 0.47 |
| QAQRGSVQN | | | | | QAQTGWVHN | | | | |
| PLNGAVHLYA | 205 | 0.376 | 2.143 | 12.85 | PLNGAVHLYA | 704 | 0.519 | 0.556 | 0.09 |
| QAQNSPVQN | | | | | QAQLSPLQN | | | | |
| PLNGAVHLYA | 206 | 0.363 | 2.131 | 24.3 | PLNGAVHLYA | 705 | 0.841 | 0.554 | 0.09 |
| QAQLQPVQN | | | | | KAQVTPVQN | | | | |
| PLNGAVHLYA | 207 | 0.550 | 2.125 | 11.68 | PLNGAVNLYA | 706 | 0.278 | 0.554 | 0.09 |
| QAQVTGVQN | | | | | QAQTQPVQN | | | | |
| PLNGAVHLYA | 208 | 0.810 | 2.121 | 11.26 | SLNGAVHLYA | 707 | 0.518 | 0.553 | 92.07 |
| QAQVMQVQN | | | | | QAQTGLVQN | | | | |
| PLNGAVHLYA | 209 | 0.697 | 2.104 | 14.86 | PLDGSVHLYA | 708 | 0.366 | 0.552 | 0.09 |
| QAQSMAVQN | | | | | QAQTGGVQN | | | | |
| PLNGAVHLYA | 210 | 0.611 | 2.104 | 4.49 | PLNGAVHLYD | 709 | 0.528 | 0.552 | 0.09 |
| QAQVGKVQN | | | | | QAQTLPVQN | | | | |
| ALNGAVHLYA | 211 | 0.652 | 2.101 | 0.09 | PLNGAVHLYA | 710 | 0.441 | 0.551 | 0.09 |
| QAQSAPVQN | | | | | QAQVSQVQK | | | | |
| PLNGAVHLYA | 212 | 0.498 | 2.101 | 3.97 | PLNGAVHLYA | 711 | 0.425 | 0.551 | 13.97 |
| QAQIQSVQN | | | | | QAQLVGVQN | | | | |
| PLNGAVHLYA | 213 | 0.537 | 2.097 | 17.15 | PLNGAVHLYA | 712 | 0.390 | 0.549 | 0.09 |
| QAQCSPVQN | | | | | QEQTSSVQN | | | | |
| PLNGAVHLYA | 214 | 0.821 | 2.079 | 3.97 | PLNGAVHIYA | 713 | 0.435 | 0.549 | 0.09 |
| QAQLQRVQN | | | | | QAQTVAVQN | | | | |
| PLNGAVHLYA | 215 | 0.330 | 2.063 | 0.09 | PLNGAVNLYA | 714 | 0.831 | 0.548 | 0.14 |
| QAQTAWVQH | | | | | QAQVTPVQN | | | | |
| PLDGAVHLYA | 216 | 0.650 | 2.062 | 0.09 | TLNGAVHLYA | 715 | 0.252 | 0.547 | 0.19 |
| QAQTGWDQN | | | | | QAQTMSVQN | | | | |
| PLNGAVHLYA | 217 | 0.411 | 2.062 | 0.89 | TLNGAVHLYA | 716 | 0.341 | 0.541 | 0.09 |
| QAQTPPVQN | | | | | QAQTLAVQN | | | | |
| PLNGAVHLYA | 218 | 0.635 | 2.054 | 1.68 | PINGAVHLYA | 717 | 0.341 | 0.540 | 0.09 |
| QAQVTKVQN | | | | | QAQVAQVQN | | | | |
| PLNGAVHLYA | 219 | 0.556 | 2.033 | 50.94 | PLNGQVHLYA | 718 | 0.885 | 0.540 | 12.57 |
| QAQSSPVQN | | | | | QAQTGWVQN | | | | |
| PLNGAVHLYA | 220 | 0.793 | 2.015 | 9.07 | QLNGAVHLYA | 719 | 0.892 | 0.540 | 39.63 |
| QAQAGPVQN | | | | | QAQTGGVQN | | | | |
| PLNGAVHLYA | 221 | 0.773 | 2.014 | 5.89 | PLNGAVHLYA | 720 | 0.542 | 0.539 | 0.09 |
| QAQLARVQN | | | | | QAQTLSVKN | | | | |
| PLNGAVHLYA | 222 | 0.789 | 2.013 | 16.36 | PLNGTVHLYA | 721 | 0.331 | 0.538 | 0.37 |
| QAQTTTVQN | | | | | QAQTGWDQN | | | | |
| PLNGAVHLYA | 223 | 0.254 | 2.011 | 0.09 | PLNGAVHLYA | 722 | 0.476 | 0.537 | 0.14 |
| QAQTGGFQN | | | | | QAQTGWFQK | | | | |
| PLNGAVHLYA | 224 | 0.657 | 2.010 | 24.44 | ALNGAVHLYA | 723 | 0.447 | 0.536 | 0.09 |
| QAQTLQVQN | | | | | QAQTSKVQN | | | | |
| PLNGAVHLYA | 225 | 0.359 | 2.005 | 63.42 | ALNGTVHLYA | 724 | 0.712 | 0.535 | 49.16 |
| QAQTMSVQN | | | | | QAQTGWVQN | | | | |
| QLNGAVHLYA | 226 | 0.675 | 2.004 | 0.09 | PLNGAVPLYA | 725 | 0.256 | 0.534 | 0.14 |
| QAQLQPVQN | | | | | QAQTGWVQH | | | | |
| PLNGAVHLYA | 227 | 0.894 | 1.994 | 10.47 | PLNGAVHLYA | 726 | 0.355 | 0.533 | 0.23 |
| QAQVAKVQN | | | | | QAKTGWVQK | | | | |
| PLNGAVHLYA | 228 | 0.217 | 1.989 | 7.24 | PLNGAVHLYA | 727 | 0.492 | 0.531 | 1.12 |
| QAQRAAVQN | | | | | QAQTPAVQN | | | | |
| PLNGAVHLYA | 229 | 0.413 | 1.988 | 38.79 | PLNGAVHLYA | 728 | 0.538 | 0.531 | 0.14 |
| QAQTVGVQN | | | | | QAQTVGVKN | | | | |
| PLNGAVHLYA | 230 | 0.381 | 1.972 | 15.84 | PLNGAVHLYA | 729 | 0.307 | 0.530 | 0.14 |
| QAQLNPVQN | | | | | QEQTGWFQN | | | | |
| PLNGAVHLYA | 231 | 0.379 | 1.971 | 26.17 | PLNGAVHLYA | 730 | 0.569 | 0.530 | 0.09 |
| QAQLSQVQN | | | | | QAQKGWDQN | | | | |
| PLNGTVHLYA | 232 | 0.421 | 1.967 | 23.37 | PLNGAVHLYD | 731 | 0.605 | 0.529 | 0.09 |
| QAQTGSVQN | | | | | QAQTSAVQN | | | | |
| PLNGAVHLYA | 233 | 0.543 | 1.960 | 12.95 | PLDGAVHLYA | 732 | 0.657 | 0.527 | 0.37 |
| QAQTKPVQN | | | | | QAQTGWVQK | | | | |
| PLNGAVHLYA | 234 | 0.603 | 1.957 | 7.71 | PLNGAVHLYA | 733 | 0.290 | 0.524 | 0.09 |
| QAQTNAVQN | | | | | QAQTAWVLN | | | | |
| ALDGAVHLYA | 235 | 0.660 | 1.955 | 21.4 | PLNGSVHLYA | 734 | 0.172 | 0.524 | 0.28 |
| QAQTGWVQN | | | | | QAQTGWVLN | | | | |
| PLNGAVHLYA | 236 | 0.303 | 1.955 | 22.06 | QLNGAVHLYA | 735 | 0.605 | 0.524 | 1.68 |
| QAQLATVQN | | | | | QAQTGWVQT | | | | |
| PLNGAVHLYA | 237 | 0.794 | 1.947 | 40.85 | PLNGAVHLYA | 736 | 0.699 | 0.524 | 0.98 |
| QAQVTPVQN | | | | | QAQVPPVQN | | | | |
| PLNGAVHLYA | 238 | 0.339 | 1.946 | 22.2 | PVNGAVHLYA | 737 | 0.734 | 0.522 | 0.09 |
| QAQVQAVQN | | | | | QAQVQSVQN | | | | |
| PLNGAVHLYA | 239 | 0.645 | 1.926 | 58.32 | QLNGAVHLYA | 738 | 0.473 | 0.522 | 0.09 |
| QAQTTSVQN | | | | | QAQTKSVQN | | | | |
| PLNGAVHLYA | 240 | 0.702 | 1.923 | 9.21 | PVNGAVHLYA | 739 | 0.825 | 0.522 | 0.23 |
| QAQCTPVQN | | | | | QAQTSSVQN | | | | |
| ALNGAVHLYA | 241 | 0.621 | 1.916 | 9.58 | PLNGAVHLYA | 740 | 0.797 | 0.520 | 11.45 |
| QAQTGRVQN | | | | | QAQATGVQN | | | | |
| PLNGSVHLYA | 242 | 0.173 | 1.908 | 0.09 | TLNGAVHLYA | 741 | 0.553 | 0.518 | 0.09 |
| QAQTGWVQD | | | | | QAQLSQVQN | | | | |
| PLNGAVHLYA | 243 | 0.555 | 1.903 | 30.47 | PLDGAVHLYA | 742 | 0.486 | 0.517 | 0.37 |
| QAQTAGVQN | | | | | QAQTVSVQN | | | | |
| PLNGAVHLYA | 244 | 0.431 | 1.902 | 29.82 | SLNGAVHLYA | 743 | 0.421 | 0.514 | 0.09 |
| QAQTSQVQN | | | | | QAQTKPVQN | | | | |
| PLNGAVHLYA | 245 | 0.443 | 1.895 | 6.4 | PLKGAVHLYA | 744 | 0.705 | 0.514 | 0.09 |
| QAQTMNVQN | | | | | QAQSAPVQN | | | | |
| PLNGAVHLYA | 246 | 0.323 | 1.885 | 34.16 | PLNGAVHLYA | 745 | 0.275 | 0.514 | 0.19 |
| QAQTSTVQN | | | | | QAKTGWVKN | | | | |
| PLNGAVHLYA | 247 | 0.712 | 1.882 | 12.43 | PLNGSVHLYA | 746 | 0.680 | 0.513 | 0.09 |
| QAQVKPVQN | | | | | QAQTLGVQN | | | | |
| PLNGAVHLYA | 248 | 0.627 | 1.878 | 24.91 | PLKGAVHLYA | 747 | 0.626 | 0.512 | 0.09 |
| QAQASPVQN | | | | | QAQLAPVQN | | | | |
| PLNGAVHLYA | 249 | 0.447 | 1.873 | 23.27 | PLNGSVHLYA | 748 | 0.207 | 0.512 | 1.17 |
| QAQVAAVQN | | | | | QAQTGWVQK | | | | |
| PLNGAVHLYA | 250 | 0.692 | 1.869 | 8.74 | PLNGAVHLYA | 749 | 0.295 | 0.511 | 0.28 |
| QAQLKSVQN | | | | | QAQTGGVQI | | | | |
| PLNGAVHLYA | 251 | 0.834 | 1.855 | 8.09 | PLNGAVHLYA | 750 | 0.440 | 0.510 | 0.37 |
| QAQIAAVQN | | | | | QPQTGWVQT | | | | |
| PLNGAVHLYA | 252 | 0.115 | 1.850 | 26.78 | PLNGAVHLYA | 751 | 0.196 | 0.508 | 4.02 |
| QAQTAAVQN | | | | | QAQTGWVLN | | | | |
| PLNGAVHLYA | 253 | 0.742 | 1.850 | 10.05 | PLNGAVHLYD | 752 | 0.611 | 0.507 | 0.14 |
| QAQTKAVQN | | | | | QAQLTPVQN | | | | |
| PLNGAVHLYA | 254 | 0.223 | 1.848 | 0.09 | QLNGAVHLYA | 753 | 0.558 | 0.507 | 0.28 |
| QAQTGSVQS | | | | | QAQTATVQN | | | | |
| PLNGAVHLYA | 255 | 0.518 | 1.845 | 7.66 | PLNGAVHLYA | 754 | 0.359 | 0.505 | 0.19 |
| QAQVSNVQN | | | | | QAQTAWGQN | | | | |
| PLNGAVHLYA | 256 | 0.690 | 1.834 | 38.65 | PLNGAVHLYA | 755 | 0.273 | 0.505 | 6.78 |
| QAQTAPVQN | | | | | QAQTGWVHN | | | | |
| PLNGAVHLYA | 257 | 0.740 | 1.833 | 28.13 | PLKGAVHLYA | 756 | 0.556 | 0.505 | 0.09 |
| QAQLMPVQN | | | | | QAQSSPVQN | | | | |
| PLNGAVHLYA | 258 | 0.798 | 1.824 | 8.88 | PLNGAVHLYA | 757 | 0.374 | 0.505 | 0.09 |
| QAQLHPVQN | | | | | QAQLGSVQT | | | | |
| PLNGAVHLYA | 259 | 0.793 | 1.823 | 5.09 | PLNGAVHLYA | 758 | 0.656 | 0.505 | 7.62 |
| QAQRAQVQN | | | | | QAQSANVQN | | | | |
| PLNGAVHLYA | 260 | 0.871 | 1.823 | 1.92 | PLNGGVHLYA | 759 | 0.866 | 0.503 | 23.27 |
| QAQLTNVQN | | | | | QAQTGSVQN | | | | |
| PLNGAVHLYA | 261 | 0.766 | 1.822 | 13.18 | PLNGAVHLYD | 760 | 0.433 | 0.503 | 0.09 |
| QAQRTTVQN | | | | | QAQRSSVQN | | | | |
| PLNGAVHLYA | 262 | 0.700 | 1.821 | 17.9 | PLNGAVHLYA | 761 | 0.164 | 0.502 | 0.19 |
| QAQTSVVQN | | | | | QAQTGGVQD | | | | |
| SLNGAVHLYA | 263 | 0.867 | 1.819 | 0.14 | PLNGAVHLYA | 762 | 0.271 | 0.502 | 0.09 |
| QAQTMSVQN | | | | | QAQTMAVKN | | | | |
| ALNGAVHLYA | 264 | 0.672 | 1.818 | 0.09 | PLNGAVHLYA | 763 | 0.325 | 0.501 | 0.23 |
| QAQTLAVQN | | | | | QAQTASVKN | | | | |
| PLNGAVHLYA | 265 | 0.736 | 1.816 | 12.2 | PLNGAVHLYA | 764 | 0.531 | 0.501 | 0.14 |
| QAQRMSVQN | | | | | QAQSSPVQK | | | | |
| ALNGAVHLYA | 266 | 0.342 | 1.813 | 0.14 | PLNGAVHLYA | 765 | 0.275 | 0.499 | 21.68 |
| QAQLTPVQN | | | | | QAQTGWVKN | | | | |
| PLNGAVHLYA | 267 | 0.785 | 1.807 | 3.65 | ALNGAVHLYA | 766 | 0.735 | 0.499 | 25.05 |
| QAQVGNVQN | | | | | QAQTGGVQN | | | | |
| PLNGAVHLYA | 268 | 0.615 | 1.805 | 4.91 | PLNGAVHLYA | 767 | 0.676 | 0.498 | 0.09 |
| QAQLMQVQN | | | | | QAQTGTVQT | | | | |
| PLNGSVHLYA | 269 | 0.550 | 1.795 | 0.09 | PLNGAVHLYA | 768 | 0.476 | 0.495 | 7.43 |
| QAQTAQVQN | | | | | QAQTGWVQI | | | | |
| PLNGAVHLYA | 270 | 0.544 | 1.793 | 24.12 | PINGAVHLYA | 769 | 0.382 | 0.494 | 0.09 |
| QAQTATVQN | | | | | QAQTSLVQN | | | | |
| PLNGAVHLYA | 271 | 0.727 | 1.790 | 7.8 | PLNGAVHIYA | 770 | 0.502 | 0.492 | 0.09 |
| QAQVHPVQN | | | | | QAQVAQVQN | | | | |
| PLNGAVHLYA | 272 | 0.836 | 1.771 | 0.09 | PLNGAVHLYA | 771 | 0.279 | 0.491 | 0.19 |
| QAQVSPVQT | | | | | QAQTVWVKN | | | | |
| PLNGAVHLYA | 273 | 0.638 | 1.766 | 9.11 | TLNGAVHLYA | 772 | 0.827 | 0.491 | 0.09 |
| QAQISSVQN | | | | | QAQLMPVQN | | | | |
| PLNGAVHLYA | 274 | 0.333 | 1.765 | 62.16 | PLNGAVHIYA | 773 | 0.672 | 0.490 | 0.09 |
| QAQVASVQN | | | | | QAQTAPVQN | | | | |
| PLNGAVHLYA | 275 | 0.347 | 1.757 | 0.09 | PLYGAVHLYA | 774 | 0.240 | 0.488 | 0.09 |
| QAQTRWDQN | | | | | QAQTASVQN | | | | |
| PLNGAVHLYA | 276 | 0.508 | 1.750 | 8.88 | TLNGAVHLYA | 775 | 0.643 | 0.488 | 0.09 |
| QAQTMTVQN | | | | | QAQLGPVQN | | | | |
| PLNGAVHLYA | 277 | 0.551 | 1.747 | 34.21 | PLDGAVHLYA | 776 | 0.637 | 0.487 | 0.42 |
| QAQRSSVQN | | | | | QAQTGWVKN | | | | |
| PLNGGVHLYA | 278 | 0.547 | 1.743 | 0.09 | PLKGAVHLYA | 777 | 0.669 | 0.487 | 0.14 |
| QAQTGWVRN | | | | | QAQVSPVQN | | | | |
| PLNGAVHLYA | 279 | 0.281 | 1.741 | 213 | PLNGAVHLYA | 778 | 0.563 | 0.486 | 9.95 |
| QAQTAWVQN | | | | | QAQSQQVQN | | | | |
| PLNGSVHLYA | 280 | 0.130 | 1.734 | 0.28 | PLNGAVDLYA | 779 | 0.497 | 0.484 | 0.09 |
| QAQPGWVQN | | | | | QAQLSPVQN | | | | |
| ALNGAVHLYA | 281 | 0.238 | 1.724 | 0.09 | PLNGAVHLYA | 780 | 0.506 | 0.484 | 0.09 |
| QAQTGWAQN | | | | | QAQVGSVQT | | | | |
| PLNGAVHLYA | 282 | 0.791 | 1.724 | 8.93 | PLNGTVHLYA | 781 | 0.405 | 0.483 | 0.14 |
| QAQRTGVQN | | | | | QAQTGWAQN | | | | |
| ALNGAVHLYA | 283 | 0.370 | 1.720 | 0.09 | ALNGAVHLYA | 782 | 0.283 | 0.482 | 0.09 |
| QAQTGWVQS | | | | | QAQTGLVQT | | | | |
| PLNGAVHLYA | 284 | 0.561 | 1.701 | 22.34 | PLNGAVHLYA | 783 | 0.562 | 0.482 | 35.42 |
| QAQVATVQN | | | | | QAQSSMVQN | | | | |
| PLNGAVHLYA | 285 | 0.480 | 1.700 | 16.68 | PLNGAVHLYA | 784 | 0.484 | 0.481 | 0.09 |
| QAQVTSVQN | | | | | KAQTAGVQN | | | | |
| PLNGAVHLYA | 286 | 0.313 | 1.699 | 74.78 | PLNGAVHLYA | 785 | 0.516 | 0.480 | 0.09 |
| QAQVSSVQN | | | | | HAQTGWVQK | | | | |
| PLNGSVHLYA | 287 | 0.353 | 1.683 | 0.14 | PLNGAVHLYA | 786 | 0.502 | 0.479 | 0.23 |
| QAQTSSVQN | | | | | QAQTSPVQK | | | | |
| PLNGAVHLYA | 288 | 0.348 | 1.682 | 18.83 | PLNGAVHLYA | 787 | 0.841 | 0.477 | 7.66 |
| QAQTNSVQN | | | | | QAQAVGVQN | | | | |
| PLNGAVHLYA | 289 | 0.815 | 1.681 | 6.03 | PLNGAVPLYA | 788 | 0.438 | 0.477 | 0.09 |
| QAQVKAVQN | | | | | QAQTGWGQN | | | | |
| PLNGAVHLYA | 290 | 0.689 | 1.666 | 19.02 | PLNGTVHLYA | 789 | 0.495 | 0.477 | 0.09 |
| QAQSGPVQN | | | | | QAQLGSVQN | | | | |
| PLNGAVHLYA | 291 | 0.660 | 1.663 | 25.61 | QLNGAVHLYA | 790 | 0.645 | 0.476 | 0.75 |
| QAQTGPVQN | | | | | QAQTGWVQH | | | | |
| PLNGAVHLYA | 292 | 0.537 | 1.660 | 22.9 | PLNGSVHLYA | 791 | 0.175 | 0.475 | 1.4 |
| QAQTAMVQN | | | | | QAQTGWVKN | | | | |
| PLNGAVHLYA | 293 | 0.358 | 1.628 | 32.11 | PLNGAVHLYD | 792 | 0.812 | 0.475 | 0.09 |
| QAQTQPVQN | | | | | QAQVAPVQN | | | | |
| PLNGSVHLYA | 294 | 0.533 | 1.625 | 32.76 | PLNGAVNLYA | 793 | 0.460 | 0.473 | 0.19 |
| QAQTGSVQN | | | | | QAQVSSVQN | | | | |
| PLNGAVHLYA | 295 | 0.519 | 1.625 | 14.44 | PLNGAVHLYA | 794 | 0.646 | 0.472 | 0.09 |
| QAQTQQVQN | | | | | QAQTLPVQK | | | | |
| PLNGAVHLYA | 296 | 0.824 | 1.620 | 9.86 | PLNGSVHLYA | 795 | 0.337 | 0.469 | 0.93 |
| QAQVSRVQN | | | | | QAQTASVQN | | | | |
| QLNGAVHLYA | 297 | 0.436 | 1.617 | 0.09 | ALNGAVHLYA | 796 | 0.294 | 0.468 | 0.37 |
| QAQVLPVQN | | | | | QAQTGWGQN | | | | |
| PLNGAVHLYA | 298 | 0.266 | 1.610 | 0.14 | PLNGAVHLYA | 797 | 0.286 | 0.468 | 7.48 |
| QAQTGWVQP | | | | | QAQTGWGQN | | | | |
| QLNGAVHLYA | 299 | 0.776 | 1.609 | 0.09 | PLNGAAHLYA | 798 | 0.451 | 0.467 | 0.09 |
| QAQLGSVQN | | | | | QAQTSPVQN | | | | |
| PLNGAVHLYA | 300 | 0.360 | 1.604 | 40.29 | PLNGAVHLYA | 799 | 0.646 | 0.465 | 0.14 |
| QAQVSAVQN | | | | | QAQTGRVQH | | | | |
| PLNGAVHLYA | 301 | 0.593 | 1.604 | 7.8 | PINGAVHLYA | 800 | 0.768 | 0.464 | 0.09 |
| QAQVLSVQN | | | | | QAQTGWVQS | | | | |
| PLNGAVHLYA | 302 | 0.861 | 1.601 | 5.05 | PLNGAVHLYA | 801 | 0.454 | 0.464 | 2.24 |
| QAQTQHVQN | | | | | QAQTPSVQN | | | | |
| PLNGAVHLYA | 303 | 0.113 | 1.590 | 52.72 | PLNGAVHLYD | 802 | 0.450 | 0.464 | 0.09 |
| QAQLASVQN | | | | | QAQTTAVQN | | | | |
| PLNGAVHLYA | 304 | 0.649 | 1.581 | 8.83 | PLNGNVHLYA | 803 | 0.415 | 0.462 | 0.09 |
| QAQQAPVQN | | | | | QAQTGWVQK | | | | |
| PLNGAVHLYA | 305 | 0.577 | 1.574 | 3.08 | PLNGAVHLYA | 804 | 0.167 | 0.462 | 2.62 |
| QAQNAQVQN | | | | | QAQTGWVEN | | | | |
| PLNGAVHLYA | 306 | 0.760 | 1.567 | 12.81 | PLNGAVNLYA | 805 | 0.371 | 0.461 | 0.19 |
| QAQATPVQN | | | | | QAQTTPVQN | | | | |
| PLNGAVHLYA | 307 | 0.544 | 1.567 | 28.13 | PLNGAVNLYA | 806 | 0.489 | 0.461 | 0.09 |
| QAQVQPVQN | | | | | QAQLSQVQN | | | | |
| PLNGAVHLYA | 308 | 0.537 | 1.563 | 33.13 | SLNGAVHLYA | 807 | 0.263 | 0.460 | 0.14 |
| QAQTTAVQN | | | | | QAQVMSVQN | | | | |
| PLNGAVHLYA | 309 | 0.455 | 1.561 | 3.46 | QLNGAVHLYA | 808 | 0.622 | 0.458 | 0.33 |
| QAQTGWVRN | | | | | QAQTGWVQS | | | | |
| PLNGAVHLYA | 310 | 0.357 | 1.561 | 21.83 | PINGAVHLYA | 809 | 0.534 | 0.457 | 0.19 |
| QAQLAAVQN | | | | | QAQTGWFQN | | | | |
| PLNGAVHLYA | 311 | 0.418 | 1.555 | 0.09 | PLNGDVHLYA | 810 | 0.419 | 0.456 | 0.14 |
| QAQTSPDQN | | | | | QAQVSSVQN | | | | |
| PLNGAVHLYA | 312 | 0.393 | 1.553 | 16.36 | PLNGGVHLYA | 811 | 0.482 | 0.456 | 0.28 |
| QAQRSGVQN | | | | | QAQTGWVQS | | | | |
| PLNGAVHLYA | 313 | 0.218 | 1.551 | 0.65 | PLNGAVNLYA | 812 | 0.559 | 0.456 | 0.09 |
| QAQTGGVQT | | | | | QAQRSTVQN | | | | |
| PLNGAVHLYA | 314 | 0.274 | 1.546 | 0.93 | ALNGAVHLYA | 813 | 0.298 | 0.455 | 0.14 |
| QAQTGWVPN | | | | | QAQTGWVQY | | | | |
| PLDSAVHLYA | 315 | 0.833 | 1.539 | 10.89 | PLNGAVHHYA | 814 | 0.170 | 0.455 | 0.09 |
| QAQTGWVQN | | | | | QAQTAAVQN | | | | |
| PLNGAVHLYA | 316 | 0.496 | 1.537 | 0.09 | PLNGAVHRYA | 815 | 0.346 | 0.454 | 0.09 |
| QAQTTPVQT | | | | | QAQTGWVQH | | | | |
| PLNGAVHLYA | 317 | 0.540 | 1.535 | 7.94 | QLNGAVHLYA | 816 | 0.627 | 0.453 | 0.37 |
| QAQLMAVQN | | | | | QAQTGWVHN | | | | |
| PLNGAVHLYA | 318 | 0.596 | 1.532 | 36.69 | PLNGAVHLYA | 817 | 0.861 | 0.452 | 56.6 |
| QAQTMPVQN | | | | | QAQRGWVQN | | | | |
| PLNGAVHHYA | 319 | 0.391 | 1.529 | 0.09 | PLNGAVHLYA | 818 | 0.233 | 0.450 | 0.14 |
| QAQTSPVQN | | | | | QAQTGSVEN | | | | |
| PLNGAVHLYA | 320 | 0.756 | 1.527 | 7.8 | ALNGAVHLYA | 819 | 0.249 | 0.450 | 0.37 |
| QAQLANVQN | | | | | QAQTGWVHN | | | | |
| PLNGAVHLYA | 321 | 0.606 | 1.520 | 11.68 | PLNGAVNLYA | 820 | 0.494 | 0.449 | 0.09 |
| QAQVSTVQN | | | | | QAQTLAVQN | | | | |
| PLNGAVHLYA | 322 | 0.602 | 1.518 | 12.9 | PLNGAVHLYA | 821 | 0.772 | 0.448 | 91.93 |
| QAQSAQVQN | | | | | QAQSSLVQN | | | | |
| PLNGAVHLYA | 323 | 0.838 | 1.508 | 8.13 | ALNGAVHLYA | 822 | 0.203 | 0.448 | 0.37 |
| QAQNTPVQN | | | | | QAQTGWLQN | | | | |
| PLNGAVHLYA | 324 | 0.381 | 1.495 | 0.09 | TLNGAVHLYA | 823 | 0.736 | 0.448 | 0.09 |
| QAQVSSVQT | | | | | QAQTLQVQN | | | | |
| PLNGAVHLYA | 325 | 0.284 | 1.492 | 0.09 | PLNGAVHLYD | 824 | 0.439 | 0.448 | 0.09 |
| QAQTVSVKN | | | | | QAQVQSVQN | | | | |
| PLNGAVHLYA | 326 | 0.710 | 1.488 | 9.21 | SLNGAVHLYA | 825 | 0.442 | 0.447 | 0.09 |
| QPQTGLVQN | | | | | QAQVTGVQN | | | | |
| PLNGAVHLYA | 327 | 0.287 | 1.487 | 621.2 | QLNGAVHLYA | 826 | 0.849 | 0.444 | 0.09 |
| QAQTGSVQN | | | | | QAQSAVVQN | | | | |
| SLNGAVHLYA | 328 | 0.437 | 1.485 | 0.09 | PLNGVVHLYA | 827 | 0.410 | 0.444 | 0.09 |
| QAQTLPVQN | | | | | QAQTGWVQT | | | | |
| PLNGAVHLYA | 329 | 0.440 | 1.478 | 0.09 | PLNGAVHLYA | 828 | 0.373 | 0.444 | 0.61 |
| QAQTGGAQN | | | | | QAQTAWVKN | | | | |
| SLNGAVHLYA | 330 | 0.618 | 1.477 | 0.09 | TLNGAVHLYA | 829 | 0.557 | 0.443 | 0.14 |
| QAQTAQVQN | | | | | QAQTLPVQN | | | | |
| PLNGAVHLYA | 331 | 0.684 | 1.466 | 14.21 | ALNGAVHLYA | 830 | 0.683 | 0.442 | 0.09 |
| QAQTAVVQN | | | | | QAQTTGVQN | | | | |
| PLNGAVHLYA | 332 | 0.533 | 1.456 | 17.71 | PLNGAVHLYA | 831 | 0.466 | 0.442 | 0.09 |
| QAQRLGVQN | | | | | KAQTMAVQN | | | | |
| PLNGSVHLYA | 333 | 0.207 | 1.453 | 0.37 | PLNGAVHLYA | 832 | 0.636 | 0.442 | 10.61 |
| QAQTGWVQH | | | | | QAQTYSVQN | | | | |
| PLNGAVHLYA | 334 | 0.489 | 1.452 | 21.26 | PLNGCVHLYA | 833 | 0.285 | 0.441 | 0.75 |
| QAQRTLVQN | | | | | QAQTGWVQN | | | | |
| SLNGAVHLYA | 335 | 0.559 | 1.451 | 0.09 | PLNGAVHLYA | 834 | 0.491 | 0.440 | 0.09 |
| QAQTMAVQN | | | | | QAQLAAVQT | | | | |
| PLNGAVHLYA | 336 | 0.707 | 1.447 | 10.23 | PLNGAVHLYA | 835 | 0.734 | 0.440 | 0.14 |
| QAQTQMVQN | | | | | QAQTALVKN | | | | |
| PLNGAVHLYA | 337 | 0.663 | 1.442 | 10.52 | PLNGAVHLYA | 836 | 0.392 | 0.439 | 0.14 |
| QAQITPVQN | | | | | KAQTVAVQN | | | | |
| PLNGAVHLYA | 338 | 0.199 | 1.434 | 0.09 | PLNGAVHLYA | 837 | 0.225 | 0.439 | 0.37 |
| QAQTVWVQK | | | | | HAQTGWVQT | | | | |
| PLNGAVHLYA | 339 | 0.584 | 1.423 | 13.97 | PLNGGVHLYA | 838 | 0.649 | 0.439 | 0.09 |
| QAQRSAVQN | | | | | QAQRGSVQN | | | | |
| PLNGAVHLYA | 340 | 0.332 | 1.420 | 99.87 | PLNGAFHLYA | 839 | 0.462 | 0.436 | 0.14 |
| QAQTASVQN | | | | | QAQTGGVQN | | | | |
| PLNGAVHLYA | 341 | 0.536 | 1.417 | 11.22 | PLNGAVHLYA | 840 | 0.496 | 0.436 | 0.09 |
| QAQTMGVQN | | | | | LAQTSPVQN | | | | |
| PLNGAVHLYA | 342 | 0.256 | 1.414 | 0.28 | PLNGAVHLYA | 841 | 0.225 | 0.435 | 2.9 |
| QAQTGGVQH | | | | | QAQTGWVQY | | | | |
| PLNGAVHLYA | 343 | 0.415 | 1.412 | 36.36 | PLNGAVHLYA | 842 | 0.538 | 0.432 | 0.19 |
| QAQVQSVQN | | | | | QAQTLPVQT | | | | |
| PLNGAVHLYA | 344 | 0.324 | 1.411 | 500.1 | PLNGAVHLYS | 843 | 0.499 | 0.432 | 0.23 |
| QAQTGGVQN | | | | | QAQTGWFQN | | | | |
| SLNGAVHLYA | 345 | 0.471 | 1.411 | 0.09 | PLNGAVHLYA | 844 | 0.696 | 0.432 | 0.33 |
| QAQTAPVQN | | | | | QAQTGLVQH | | | | |
| PLNGAVHLYA | 346 | 0.680 | 1.410 | 16.68 | PLNGAVHLYA | 845 | 0.628 | 0.432 | 0.98 |
| QAQISPVQN | | | | | QAQTPTVQN | | | | |
| PLNGAVHLYA | 347 | 0.345 | 1.409 | 0.09 | PLYGAVHLYA | 846 | 0.274 | 0.429 | 0.28 |
| QPQTGWVKN | | | | | QAQTAWVQN | | | | |
| PLNGAVHLYA | 348 | 0.242 | 1.409 | 0.14 | PLNGAVHLYA | 847 | 0.275 | 0.428 | 0.23 |
| QAQTGSAQN | | | | | KAQTASVQN | | | | |
| PLNGAVHLYA | 349 | 0.474 | 1.393 | 2.62 | PLNGAVHLYA | 848 | 0.285 | 0.428 | 0.19 |
| QAQTGWAQN | | | | | QAQTASVQK | | | | |
| PLNGAVHLYA | 350 | 0.383 | 1.392 | 0.09 | PLDGSVHLYA | 849 | 0.641 | 0.424 | 0.09 |
| QAQTMSVQT | | | | | QAQTAWVQN | | | | |
| PLNGSVHLYA | 351 | 0.649 | 1.391 | 12.29 | PLNGADHLYA | 850 | 0.540 | 0.423 | 0.09 |
| QAQTAWVQN | | | | | QAQTSSVQN | | | | |
| PLNGAVHLYA | 352 | 0.410 | 1.387 | 8.13 | PLNGAVHLYA | 851 | 0.594 | 0.422 | 0.09 |
| QAQVGGVQN | | | | | QAQTSPVQI | | | | |
| PLNGAVHLYA | 353 | 0.496 | 1.387 | 0.09 | PLNGAVHLYA | 852 | 0.661 | 0.420 | 40 |
| KAQTSPVQN | | | | | QAQIGWVQN | | | | |
| PLNGAVHLYA | 354 | 0.768 | 1.381 | 0.09 | PLNGAVHLYA | 853 | 0.209 | 0.419 | 0.09 |
| QAQLAPVQT | | | | | KAQTSTVQN | | | | |
| PLDGAVHLYA | 355 | 0.901 | 1.375 | 0.09 | PLNGAVHRYA | 854 | 0.237 | 0.419 | 0.19 |
| QAQTALVQN | | | | | QAQTGWVQT | | | | |
| PLNGAVHLYA | 356 | 0.594 | 1.368 | 58.98 | PLNGAVHLYD | 855 | 0.268 | 0.419 | 0.14 |
| QAQTALVQN | | | | | QAQTSTVQN | | | | |
| PLNGAVHLYA | 357 | 0.470 | 1.366 | 19.63 | QLNGAVHLYA | 856 | 0.568 | 0.418 | 69.31 |
| QAQLAGVQN | | | | | QAQTGLVQN | | | | |
| PLNGAVHLYA | 358 | 0.613 | 1.365 | 9.53 | PLNGAVNLYA | 857 | 0.864 | 0.417 | 0.09 |
| QAQRTAVQN | | | | | QAQLGPVQN | | | | |
| PLNGAVHLYA | 359 | 0.540 | 1.363 | 22.15 | PLNGAVHLYA | 858 | 0.400 | 0.417 | 0.23 |
| QAQRSPVQN | | | | | QAQTGWDKN | | | | |
| PLNGAVHLYA | 360 | 0.513 | 1.360 | 39.82 | PLNGALHLYA | 859 | 0.432 | 0.416 | 0.14 |
| QAQTLAVQN | | | | | QAQTGWVKN | | | | |
| PLNGAVHLYA | 361 | 0.322 | 1.351 | 5.42 | PLNGNVHLYA | 860 | 0.698 | 0.414 | 0.09 |
| QAQLAHVQN | | | | | HAQTGWVQN | | | | |
| PLNGAVHLYA | 362 | 0.352 | 1.348 | 36.69 | PLNGAVHLYA | 861 | 0.861 | 0.414 | 8.23 |
| QAQTSLVQN | | | | | QAQKSSVQN | | | | |
| PLNGAVHLYA | 363 | 0.548 | 1.347 | 30.75 | PLNGAVHLYA | 862 | 0.691 | 0.412 | 30.7 |
| QAQRLSVQN | | | | | QAQTGYVQN | | | | |
| PLNGAVHLYD | 364 | 0.406 | 1.344 | 0.09 | PVNGAVHLYA | 863 | 0.456 | 0.412 | 0.09 |
| QAQLSPVQN | | | | | QAQVGSVQN | | | | |
| PLNGAVHLYA | 365 | 0.424 | 1.339 | 13.97 | PLNGGVHLYA | 864 | 0.536 | 0.411 | 1.17 |
| QAQLMGVQN | | | | | QAQTGWVKN | | | | |
| PLNGSVHLYA | 366 | 0.147 | 1.336 | 1.03 | PLNGAVHLYA | 865 | 0.627 | 0.410 | 0.09 |
| QAQTGWVQT | | | | | QAQRGWVQT | | | | |
| PLNGAVHLYA | 367 | 0.588 | 1.335 | 11.26 | PLNGAVNLYA | 866 | 0.729 | 0.410 | 0.09 |
| QAQSMQVQN | | | | | QAQRSLVQN | | | | |
| PLNGGVHLYA | 368 | 0.483 | 1.334 | 0.14 | PINGAVHLYA | 867 | 0.358 | 0.410 | 0.51 |
| QAQTGWFQN | | | | | QAQTGWDQN | | | | |
| PLNGAVHLYA | 369 | 0.834 | 1.326 | 14.02 | PLNGNVHLYA | 868 | 0.536 | 0.410 | 0.23 |
| QAQTQTVQN | | | | | QAQTGWVQT | | | | |
| PLDGAVHLYS | 370 | 0.860 | 1.323 | 9.44 | PLNGAVHLYA | 869 | 0.265 | 0.409 | 0.23 |
| QAQTGWVQN | | | | | KAQTGWFQN | | | | |
| PLNGAVHLYA | 371 | 0.325 | 1.319 | 0.14 | PLNGAVHLYA | 870 | 0.421 | 0.408 | 0.09 |
| QAQTGWEQN | | | | | QEQTSTVQN | | | | |
| PLNGAVHLYA | 372 | 0.487 | 1.317 | 29.44 | PLNGAVHLYA | 871 | 0.281 | 0.406 | 0.14 |
| QAQVGSVQN | | | | | QAQTGGVEN | | | | |
| PLNGAVHLYA | 373 | 0.339 | 1.317 | 24.86 | PLNGAVHLYS | 872 | 0.831 | 0.406 | 0.09 |
| QAQVSGVQN | | | | | QAQTGWVQI | | | | |
| PLNGAVHLYA | 374 | 0.767 | 1.312 | 13.04 | SLNGAVHLYA | 873 | 0.480 | 0.406 | 0.09 |
| QAQVMAVQN | | | | | QAQTGLVQK | | | | |
| PLNGAVHLYA | 375 | 0.814 | 1.309 | 23.37 | PLNGAVHLYA | 874 | 0.605 | 0.403 | 0.09 |
| QAQIGGVQN | | | | | QAQTGCVRN | | | | |
| PLNGAVHLYA | 376 | 0.841 | 1.301 | 5.47 | PLNGADHLYA | 875 | 0.758 | 0.402 | 0.09 |
| QAQIAGVQN | | | | | QAQVTPVQN | | | | |
| PLNGAVHLYA | 377 | 0.200 | 1.297 | 0.09 | PLNGAVHLYA | 876 | 0.129 | 0.400 | 0.28 |
| KAQTVSVQN | | | | | PAQTGWVQT | | | | |
| PLNGAVHLYS | 378 | 0.838 | 1.297 | 4.3 | PLNGDVHLYA | 877 | 0.900 | 0.400 | 0.09 |
| QAQTGRVQN | | | | | QAQTSAVQN | | | | |
| PLNGAVHLYA | 379 | 0.826 | 1.296 | 7.34 | TLNGAVHLYA | 878 | 0.635 | 0.399 | 0.09 |
| QAQLSHVQN | | | | | QAQSSLVQN | | | | |
| PLNGAVHLYA | 380 | 0.865 | 1.288 | 4.77 | SLNGAVHLYA | 879 | 0.645 | 0.399 | 0.14 |
| QAQVQTVQN | | | | | QAQRSVVQN | | | | |
| SLDGAVHLYA | 381 | 0.875 | 1.287 | 61.08 | SLNGAVHLYA | 880 | 0.817 | 0.397 | 0.33 |
| QAQTGWVQN | | | | | QAQTGWVQS | | | | |
| PLNGAVHLYA | 382 | 0.326 | 1.287 | 0.09 | PVNGAVHLYA | 881 | 0.800 | 0.397 | 0.14 |
| QPQTGWDQN | | | | | QAQTGWVQS | | | | |
| PLNGAVHLYA | 383 | 0.564 | 1.284 | 11.36 | PLNGAVHLYA | 882 | 0.619 | 0.396 | 0.09 |
| QAQRNSVQN | | | | | QAQLSPVQS | | | | |
| PLNGAVHLYA | 384 | 0.533 | 1.283 | 57.16 | PLNGAVHLYA | 883 | 0.285 | 0.395 | 0.09 |
| QAQTLPVQN | | | | | QPQTGWLQN | | | | |
| PLNGAVHLYA | 385 | 0.569 | 1.282 | 5.09 | PLNGAVNLYA | 884 | 0.719 | 0.394 | 0.14 |
| QAQTKQVQN | | | | | QAQSAPVQN | | | | |
| PLNGSVHLYA | 386 | 0.144 | 1.279 | 972 | PLNGTVHLYA | 885 | 0.384 | 0.394 | 0.09 |
| QAQTGWVQN | | | | | QAQTGWVPN | | | | |
| PLNGAVHLYA | 387 | 0.593 | 1.278 | 11.17 | PVDGAVHLYA | 886 | 0.792 | 0.394 | 0.23 |
| QAQLGQVQN | | | | | QAQTGSVQN | | | | |
| SLNGAVHLYA | 388 | 0.684 | 1.277 | 19.35 | PLNGAVHLYD | 887 | 0.261 | 0.393 | 0.09 |
| QAQTGRVQN | | | | | QAQTAAVQN | | | | |
| PLDGAVHLYA | 389 | 0.647 | 1.275 | 263.1 | PLKGAVHLYA | 888 | 0.352 | 0.392 | 0.09 |
| QAQTGWVQN | | | | | QAQTSTVQN | | | | |
| PLNGSVHLYA | 390 | 0.181 | 1.273 | 0.19 | PLNGAVHLYA | 889 | 0.730 | 0.392 | 0.14 |
| QAQTGWAQN | | | | | QAQTGPVQT | | | | |
| TLNGAVHLYA | 391 | 0.405 | 1.266 | 0.09 | PLNGAVHLYA | 890 | 0.432 | 0.392 | 0.14 |
| QAQVASVQN | | | | | QAQTVGVQK | | | | |
| PLNGAVHLYA | 392 | 0.733 | 1.261 | 2.85 | PLNGAVNLYA | 891 | 0.451 | 0.391 | 0.14 |
| QAQNMQVQN | | | | | QAQTSTVQN | | | | |
| PLNGAVHLYA | 393 | 0.863 | 1.258 | 3.18 | PLNGAVHLYA | 892 | 0.414 | 0.390 | 0.09 |
| QAQNVQVQN | | | | | RAQTVSVQN | | | | |
| PLNGAVHLYA | 394 | 0.299 | 1.250 | 0.14 | PLNGAVHLYA | 893 | 0.431 | 0.390 | 0.09 |
| QAQTGSVQI | | | | | QAQTASGQN | | | | |
| PLNGTVHLYA | 395 | 0.840 | 1.249 | 15.75 | PLNGAVHLYA | 894 | 0.631 | 0.390 | 20.28 |
| QAQTGGVQN | | | | | QAQSVSVQN | | | | |
| PLNGAVHLYA | 396 | 0.805 | 1.248 | 0.09 | PLNGAVHLYA | 895 | 0.724 | 0.389 | 0.09 |
| KAQVSPVQN | | | | | QAQSGPVQT | | | | |
| PLNGAVHLYA | 397 | 0.765 | 1.241 | 6.4 | QLNGAVHLYA | 896 | 0.578 | 0.389 | 0.47 |
| QAQRGGVQN | | | | | QAQTGWGQN | | | | |
| PLNGGVHLYA | 398 | 0.335 | 1.240 | 25.94 | PLNGAVHLYA | 897 | 0.670 | 0.386 | 0.09 |
| QAQTGLVQN | | | | | QAQVMSVKN | | | | |
| PLNGAVHLYA | 399 | 0.542 | 1.237 | 33.32 | PLNGTVHLYA | 898 | 0.557 | 0.386 | 0.09 |
| QAQVMSVQN | | | | | KAQTGSVQN | | | | |
| PLNGAVHLYA | 400 | 0.401 | 1.235 | 15.05 | PLNGAVNLYA | 899 | 0.527 | 0.385 | 0.09 |
| QAQTTGVQN | | | | | QAQTSLVQN | | | | |
| PLNGAVHLYA | 401 | 0.375 | 1.229 | 0.09 | PINGAVHLYA | 900 | 0.867 | 0.385 | 0.14 |
| QAQLSPVQK | | | | | QAQTLSVQN | | | | |
| SLNGAVHLYA | 402 | 0.677 | 1.226 | 0.09 | PLNGAVHLYA | 901 | 0.458 | 0.384 | 0.09 |
| QAQTAAVQN | | | | | QAQLGSVQK | | | | |
| PINGAVHLYA | 403 | 0.351 | 1.223 | 0.14 | TLNGAVHLYA | 902 | 0.728 | 0.383 | 0.09 |
| QAQTSPVQN | | | | | QAQTAMVQN | | | | |
| PLNGAVHLYA | 404 | 0.691 | 1.220 | 15.28 | PLNGAVHLYA | 903 | 0.775 | 0.383 | 0.09 |
| QAQLSRVQN | | | | | QAHLSSVQN | | | | |
| PLNGAVHLYA | 405 | 0.259 | 1.213 | 0.09 | PLNGAVHLYA | 904 | 0.552 | 0.380 | 0.09 |
| QAQVSSVQK | | | | | KAQVMSVQN | | | | |
| PLNGAVHLYA | 406 | 0.768 | 1.212 | 0.09 | PLNGAVHLYA | 905 | 0.569 | 0.380 | 0.09 |
| QAQTAPVQT | | | | | QAQTGLVLN | | | | |
| PLNGAVHLYA | 407 | 0.740 | 1.206 | 7.9 | PLNGGVHLYA | 906 | 0.491 | 0.378 | 1.03 |
| QAQMAPVQN | | | | | QAQTGWVQK | | | | |
| PLNGAVHLYA | 408 | 0.499 | 1.202 | 7.06 | PLNGAVHLYA | 907 | 0.250 | 0.378 | 0.61 |
| QAQILGVQN | | | | | KAQTGWVQK | | | | |
| PLNGAVHLYA | 409 | 0.216 | 1.198 | 0.14 | PLNGAVLLYA | 908 | 0.482 | 0.378 | 0.09 |
| QAQTASVQT | | | | | QAQTASVQN | | | | |
| PLNGAVHLYA | 410 | 0.267 | 1.196 | 0.23 | PLNGDVHLYA | 909 | 0.507 | 0.378 | 0.09 |
| QAQTGSLQN | | | | | QAQTMAVQN | | | | |
| PLNGAVHLYA | 411 | 0.638 | 1.196 | 28.79 | PLNGAVHLYA | 910 | 0.258 | 0.377 | 0.09 |
| QAQTGTVQN | | | | | KAQTAAVQN | | | | |
| PLNGGVHLYA | 412 | 0.578 | 1.193 | 565.1 | PLNGAVHLYA | 911 | 0.549 | 0.377 | 763.5 |
| QAQTGWVQN | | | | | QAQTGLVQN | | | | |
| PLNGAVHLYA | 413 | 0.237 | 1.183 | 0.89 | PLNGDVHLYA | 912 | 0.699 | 0.376 | 0.14 |
| QAQTGSVQT | | | | | QAQSSPVQN | | | | |
| PLNGAVHLYA | 414 | 0.353 | 1.171 | 0.09 | PLNGAVHLYA | 913 | 0.832 | 0.376 | 14.96 |
| QAQTSSVQT | | | | | QAQTSNVQN | | | | |
| PLNGAVHLYA | 415 | 0.414 | 1.167 | 10.84 | ALDGAVHLYA | 914 | 0.702 | 0.376 | 0.47 |
| QAQTSHVQN | | | | | QAQTGSVQN | | | | |
| PLNGGVHLYA | 416 | 0.561 | 1.166 | 0.28 | PLNGAVHLYS | 915 | 0.848 | 0.375 | 0.09 |
| QAQTGWDQN | | | | | QAQTSSVQN | | | | |
| PLNGAVHLYA | 417 | 0.753 | 1.165 | 3.55 | PLKGAVHLYA | 916 | 0.390 | 0.373 | 0.19 |
| QAQRIGVQN | | | | | QAQLSPVQN | | | | |
| TLNGAVHLYA | 418 | 0.771 | 1.164 | 0.09 | LLNGAVHLYA | 917 | 0.349 | 0.373 | 0.09 |
| QAQTTSVQN | | | | | QAQTVSVQN | | | | |
| PLNGAVHLYA | 419 | 0.347 | 1.158 | 0.14 | PLNGGVHLYA | 918 | 0.571 | 0.373 | 0.37 |
| QAQTGWGQT | | | | | QAQTGWVQI | | | | |
| PLNGAVHLYA | 420 | 0.549 | 1.158 | 14.3 | PLNGAVHLYS | 919 | 0.863 | 0.372 | 0.14 |
| QAQSAMVQN | | | | | QAQTGWVRN | | | | |
| PLNGAVHLYA | 421 | 0.510 | 1.156 | 23.23 | PLNGAVHLYA | 920 | 0.426 | 0.371 | 0.09 |
| QAQTSMVQN | | | | | QAQTGWGHN | | | | |
| PLNGAVHLYA | 422 | 0.781 | 1.148 | 13.09 | PHNGAVHLYA | 921 | 0.849 | 0.371 | 0.09 |
| QAQSMGVQN | | | | | QAQTTSVQN | | | | |
| PLNGAVHLYA | 423 | 0.568 | 1.145 | 27.62 | PLNRAVHLYA | 922 | 0.436 | 0.371 | 0.09 |
| QAQSMSVQN | | | | | QAQTGWVQK | | | | |
| PLNGAVHLYA | 424 | 0.442 | 1.145 | 94.03 | PLNGAVHLYD | 923 | 0.509 | 0.371 | 0.09 |
| QAQTSSVQN | | | | | QAQRASVQN | | | | |
| PLNGAVHLYA | 425 | 0.620 | 1.143 | 30.52 | PLNGAVHLYS | 924 | 0.800 | 0.370 | 584.7 |
| QAQTSGVQN | | | | | QAQTGWVQN | | | | |
| PLNGAVHLYA | 426 | 0.557 | 1.141 | 22.9 | PLNGSVHLYA | 925 | 0.401 | 0.370 | 0.42 |
| QAQTGAVQN | | | | | QAQTGTVQN | | | | |
| PLNGAVHLYA | 427 | 0.627 | 1.140 | 14.02 | PLNGAVHLYD | 926 | 0.752 | 0.370 | 0.09 |
| QAQVNSVQN | | | | | QAQTSGVQN | | | | |
| PLNGAVHLYA | 428 | 0.804 | 1.139 | 12.48 | PLNGAVHLYA | 927 | 0.389 | 0.368 | 0.19 |
| QAQVAGVQN | | | | | QAKTGWFQN | | | | |
| PLNGAVHLYA | 429 | 0.710 | 1.139 | 27.01 | KLDGAVHLYA | 928 | 0.763 | 0.367 | 0.14 |
| QAQIGSVQN | | | | | QAQTGLVQN | | | | |
| PLDGAVHVYA | 430 | 0.818 | 1.132 | 9.49 | PLNGAVHLYA | 929 | 0.581 | 0.363 | 9.39 |
| QAQTGWVQN | | | | | QAQSQMVQN | | | | |
| TLNGAVHLYA | 431 | 0.474 | 1.131 | 0.09 | TLNGAVHLYA | 930 | 0.706 | 0.363 | 0.09 |
| QAQTTPVQN | | | | | QAQTSVVQN | | | | |
| PLNGAVHLYA | 432 | 0.442 | 1.131 | 41.64 | PLNGAVHLYA | 931 | 0.449 | 0.361 | 0.09 |
| QAQLGSVQN | | | | | KAQTGTVQN | | | | |
| PLNGAVHLYA | 433 | 0.807 | 1.130 | 7.06 | PLNGAVHLYA | 932 | 0.440 | 0.361 | 0.09 |
| QAQVNGVQN | | | | | QAQTVSVHN | | | | |
| QLNGAVHLYA | 434 | 0.857 | 1.127 | 0.19 | PLNGAVHLYA | 933 | 0.559 | 0.361 | 0.19 |
| QAQLSSVQN | | | | | QAQLSSVQT | | | | |
| PLNGAVHLYA | 435 | 0.652 | 1.127 | 7.62 | PVNGAVHLYA | 934 | 0.791 | 0.359 | 0.28 |
| QAQLTAVQN | | | | | QAQTGWFQN | | | | |
| PLNGAVHLYA | 436 | 0.892 | 1.125 | 3.22 | PLNGAVHLYA | 935 | 0.266 | 0.356 | 0.09 |
| QAQVQNVQN | | | | | QAQTASVRN | | | | |
| PLNGAVHLYA | 437 | 0.737 | 1.117 | 14.63 | PLNGDVHLYA | 936 | 0.410 | 0.355 | 0.09 |
| QAQTKSVQN | | | | | QAQTSTVQN | | | | |
| PLNGAVHLYA | 438 | 0.847 | 1.117 | 16.64 | PLNGAVHLYA | 937 | 0.477 | 0.354 | 0.09 |
| QAQSVGVQN | | | | | KAQLGSVQN | | | | |
| QLNGAVHLYA | 439 | 0.413 | 1.116 | 0.09 | PINGAVHLYA | 938 | 0.774 | 0.354 | 0.09 |
| QAQLGPVQN | | | | | QAQLVPVQN | | | | |
| PLNGAVHLYA | 440 | 0.274 | 1.107 | 0.47 | PLNGAVHHYA | 939 | 0.833 | 0.354 | 0.09 |
| QAQTAWVQT | | | | | QAQLGPVQN | | | | |
| PLNGAVHLYA | 441 | 0.651 | 1.099 | 11.54 | PLNGAVHLYA | 940 | 0.387 | 0.353 | 0.19 |
| QAQNASVQN | | | | | PAQTGWVQH | | | | |
| PLNGAVHLYA | 442 | 0.661 | 1.098 | 45.57 | PLNGAVHLYA | 941 | 0.696 | 0.353 | 0.56 |
| QAQTSAVQN | | | | | QAQVPAVQN | | | | |
| ALNGAVHLYA | 443 | 0.530 | 1.097 | 0.09 | PLNGAVNLYA | 942 | 0.774 | 0.353 | 0.09 |
| QAQLAAVQN | | | | | QAQRLSVQN | | | | |
| PLNGAVHLYD | 444 | 0.721 | 1.097 | 0.09 | PLNGAVHLYA | 943 | 0.534 | 0.352 | 0.23 |
| QAQVSPVQN | | | | | QAQTGRVQK | | | | |
| PLNGAVHLYA | 445 | 0.442 | 1.095 | 0.09 | PLNGAVLLYA | 944 | 0.370 | 0.352 | 0.09 |
| QAQTMSVKN | | | | | QAQTVSVQN | | | | |
| PLNGAVHLYA | 446 | 0.650 | 1.092 | 7.71 | PLNGAVHLYA | 945 | 0.510 | 0.351 | 11.4 |
| QAQTANVQN | | | | | QAQAMSVQN | | | | |
| PLNGAVHLYA | 447 | 0.358 | 1.092 | 5.19 | PLNGAVHLYA | 946 | 0.451 | 0.351 | 0.23 |
| QAQTGWFQN | | | | | QAQTAWVHN | | | | |
| PLNGNVHLYA | 448 | 0.392 | 1.090 | 74.03 | PLNGAVHLYA | 947 | 0.615 | 0.350 | 0.09 |
| QAQTGWVQN | | | | | QPQTGWVRN | | | | |
| PLNGSVHLYA | 449 | 0.195 | 1.087 | 0.23 | PLNGAVHLYA | 948 | 0.548 | 0.350 | 0.09 |
| QAQTGWVQS | | | | | QEQTGWLQN | | | | |
| PLNGAVHLYA | 450 | 0.279 | 1.081 | 0.09 | PLNGAVHLYA | 949 | 0.535 | 0.348 | 0.09 |
| QAQTGGVLN | | | | | KAQTSAVQN | | | | |
| PLNGAVHLYA | 451 | 0.888 | 1.081 | 16.45 | PLNGSVHLYA | 950 | 0.126 | 0.348 | 0.98 |
| QAQLTSVQN | | | | | QAQTVWVQN | | | | |
| PLNGAVHLYA | 452 | 0.176 | 1.079 | 19.3 | PLNGTVHLYA | 951 | 0.330 | 0.347 | 0.33 |
| QAQTGWVQT | | | | | QAQTGWFQN | | | | |
| PLNGAVHLYA | 453 | 0.804 | 1.078 | 10.7 | PLNGAVNLYA | 952 | 0.375 | 0.347 | 0.23 |
| QAQRSQVQN | | | | | QAQVASVQN | | | | |
| PLNGAVHIYA | 454 | 0.840 | 1.074 | 0.09 | PLNGAVHLYA | 953 | 0.674 | 0.347 | 0.19 |
| QAQVSPVQN | | | | | QAQTGLVRN | | | | |
| PLNGSVHLYA | 455 | 0.130 | 1.062 | 0.37 | PLNGAVNLYA | 954 | 0.587 | 0.346 | 0.09 |
| QAQTGWFQN | | | | | QAQLSTVQN | | | | |
| PLNGAVHLYA | 456 | 0.863 | 1.060 | 3.13 | PLNGAVHLYA | 955 | 0.721 | 0.346 | 0.09 |
| QAQRIPVQN | | | | | KAQTQPVQN | | | | |
| QLNGAVHLYA | 457 | 0.718 | 1.054 | 0.09 | PLNGTVHLYA | 956 | 0.382 | 0.346 | 0.33 |
| QAQLVPVQN | | | | | QAQTGWGQN | | | | |
| PLNGAVHLYA | 458 | 0.198 | 1.052 | 2.06 | PLNGAVHLYA | 957 | 0.453 | 0.345 | 0.09 |
| QAQTGWVQD | | | | | QAQRTLVQT | | | | |
| PLNGAVHLYA | 459 | 0.357 | 1.051 | 0.09 | PLNGAVHLYA | 958 | 0.157 | 0.345 | 0.6 |
| KAQVSSVQN | | | | | QAQTGSIQN | | | | |
| PLNGAVHLYA | 460 | 0.721 | 1.051 | 11.54 | QLNGAVHLYA | 959 | 0.574 | 0.344 | 1.4 |
| QAQRVEVQN | | | | | QAQTGWDQN | | | | |
| PLNGAVHLYA | 461 | 0.553 | 1.048 | 56.6 | PLNGAVHLYA | 960 | 0.599 | 0.344 | 0.14 |
| QAQVLPVQN | | | | | QAQLSSVKN | | | | |
| PLNGSVHLYA | 462 | 0.194 | 1.044 | 0.28 | QLNGAVHLYA | 961 | 0.451 | 0.343 | 1.45 |
| QAQTGWVRN | | | | | QAQTVSVQN | | | | |
| PLNGTVHLYA | 463 | 0.648 | 1.040 | 0.09 | PLNGAVHLYA | 962 | 0.726 | 0.343 | 0.09 |
| QAQSSPVQN | | | | | QAQTLQVQK | | | | |
| PLNGAVHLYA | 464 | 0.301 | 1.039 | 0.42 | TLNGAVHLYA | 963 | 0.573 | 0.343 | 0.14 |
| QAQTGGVQK | | | | | QAQVLPVQN | | | | |
| PLNGAVHLYA | 465 | 0.727 | 1.039 | 14.63 | PLNGAVHLYA | 964 | 0.756 | 0.342 | 0.09 |
| QAQSVAVQN | | | | | QAQVGSVKN | | | | |
| QLNGAVHLYA | 466 | 0.815 | 1.030 | 0.09 | PLNGAVHLYA | 965 | 0.744 | 0.341 | 0.09 |
| QAQLSGVQN | | | | | QAQLAPLQN | | | | |
| PLNGAVHLYA | 467 | 0.384 | 1.026 | 11.26 | QLNGAVHLYA | 966 | 0.716 | 0.341 | 0.09 |
| QAQLQQVQN | | | | | QAQTGSVKN | | | | |
| PLNGAVHLYA | 468 | 0.547 | 1.025 | 32.06 | PLNGAVHHYA | 967 | 0.620 | 0.341 | 0.09 |
| QAQLSTVQN | | | | | QAQRSTVQN | | | | |
| PLNGAVHLYA | 469 | 0.813 | 1.025 | 18.09 | QLNGSVHLYA | 968 | 0.266 | 0.341 | 0.23 |
| QAQSAAVQN | | | | | QAQTGRVQN | | | | |
| SLNGAVHLYA | 470 | 0.774 | 1.014 | 0.14 | TLNGAVHLYA | 969 | 0.818 | 0.340 | 0.09 |
| QAQVSAVQN | | | | | QAQVKPVQN | | | | |
| PLNGAVHLYA | 471 | 0.432 | 1.013 | 20.47 | PLNGTVHLYA | 970 | 0.784 | 0.339 | 24.58 |
| QAQRTSVQN | | | | | QAQTGLVQN | | | | |
| PLNGAVHLYA | 472 | 0.877 | 1.012 | 5.47 | PLNGAVHLYA | 971 | 0.396 | 0.338 | 0.65 |
| QAQTQNVQN | | | | | QAQTPGVQN | | | | |
| PLNGAVHLYA | 473 | 0.877 | 1.007 | 4.81 | PINGAVHLYA | 972 | 0.713 | 0.338 | 0.28 |
| QAQVNAVQN | | | | | QAQTSSVQN | | | | |
| PLNGAVHLYA | 474 | 0.585 | 1.006 | 0.09 | PLNGAVHLYA | 973 | 0.428 | 0.337 | 0.09 |
| QAQTVAVQT | | | | | QAQLSAVQT | | | | |
| PLNGAVHLYA | 475 | 0.487 | 1.005 | 20.75 | PLNGAVHLYS | 974 | 0.446 | 0.337 | 0.47 |
| QAQLMSVQN | | | | | QAQTGWDQN | | | | |
| PLNGAVHLYA | 476 | 0.179 | 1.000 | 15655 | PLNCAVHLYA | 975 | 0.856 | 0.336 | 14.39 |
| QAQTGWVQN | | | | | QAQTGWVQN | | | | |
| PLNGAVHLYA | 477 | 0.354 | 0.999 | 0.09 | PLNGAVNLYA | 976 | 0.624 | 0.336 | 0.14 |
| HAQTGWVQH | | | | | QAQTMPVQN | | | | |
| PLNGAVHLYA | 478 | 0.283 | 0.997 | 7.48 | PLNGAVHLYS | 977 | 0.283 | 0.335 | 0.09 |
| QAQTGWVQH | | | | | QAQTVAVQN | | | | |
| PLNGAVHHYA | 479 | 0.408 | 0.993 | 0.09 | PLNGTVHLYA | 978 | 0.366 | 0.334 | 0.79 |
| QAQTMSVQN | | | | | QAQTGWVKN | | | | |
| PLNGAVHLYA | 480 | 0.228 | 0.978 | 3.08 | PLNGGVHLYA | 979 | 0.465 | 0.333 | 0.37 |
| QAQTGWVQS | | | | | QAQTGWVHN | | | | |
| PLNGAVHLYD | 481 | 0.409 | 0.976 | 0.09 | QLNGAVHLYA | 980 | 0.558 | 0.332 | 1.59 |
| QAQTTPVQN | | | | | QAQTGWVQK | | | | |
| PLNGAVHLYA | 482 | 0.837 | 0.973 | 3.69 | PLNGAVQLYA | 981 | 0.524 | 0.331 | 0.09 |
| QAQTMMVQN | | | | | QAQTLAVQN | | | | |
| PLNGAVHLYA | 483 | 0.293 | 0.971 | 0.14 | PLNGTVHLYA | 982 | 0.695 | 0.331 | 0.09 |
| QAQTGGVQS | | | | | QAQVGSVQN | | | | |
| PLNGAVNLYA | 484 | 0.499 | 0.969 | 0.14 | PLNGGVHLYA | 983 | 0.764 | 0.330 | 17.67 |
| QAQTSPVQN | | | | | QAQTGGVQN | | | | |
| ALNGAVHLYA | 485 | 0.322 | 0.968 | 47.95 | PLKGAVHLYA | 984 | 0.474 | 0.330 | 0.09 |
| QAQTGLVQN | | | | | QAQVSQVQN | | | | |
| ALNGAVHLYA | 486 | 0.247 | 0.964 | 0.09 | PLNGAVHHYA | 985 | 0.526 | 0.330 | 0.09 |
| QAQVATVQN | | | | | QAQTQPVQN | | | | |
| PLNGAVHLYA | 487 | 0.258 | 0.959 | 0.23 | PLNGAVHLYA | 986 | 0.156 | 0.329 | 0.33 |
| QAQTGSFQN | | | | | QAPTGWVQT | | | | |
| PLNGAVHLYA | 488 | 0.422 | 0.958 | 27.01 | PLNGAVNLYA | 987 | 0.343 | 0.329 | 0.33 |
| QAQRSVVQN | | | | | QAQTMSVQN | | | | |
| PLNGAVHLYA | 489 | 0.224 | 0.957 | 0.65 | PLNGAVHLYA | 988 | 0.681 | 0.329 | 29.16 |
| QAQTGSVQK | | | | | QAQTGVVQN | | | | |
| PLNGAVHLYA | 490 | 0.503 | 0.954 | 16.31 | PLNGAVHLYA | 989 | 0.622 | 0.328 | 0.42 |
| QAQTQLVQN | | | | | QAQVPQVQN | | | | |
| PLNGAVHLYA | 491 | 0.252 | 0.954 | 0.19 | PHNGAVHLYA | 990 | 0.295 | 0.327 | 0.14 |
| QAQTGGVHN | | | | | QAQTMSVQN | | | | |
| PLNGAVHLYA | 492 | 0.328 | 0.953 | 11.92 | QLNGAVHLYA | 991 | 0.493 | 0.327 | 0.56 |
| QAQRTPVQN | | | | | QAQTGWLQN | | | | |
| PLNGAVHLYA | 493 | 0.852 | 0.952 | 5.33 | PLNGAVHLYA | 992 | 0.118 | 0.327 | 1.03 |
| QAQRQQVQN | | | | | QAQTGCVQK | | | | |
| PLNGAVHLYA | 494 | 0.321 | 0.952 | 0.19 | SLNGAVHLYA | 993 | 0.712 | 0.327 | 0.28 |
| QAQTGSVRN | | | | | QAQTSLVQN | | | | |
| PLNGAVHLYA | 495 | 0.758 | 0.946 | 11.87 | PLNGAVHLYA | 994 | 0.422 | 0.326 | 0.23 |
| QAQVQGVQN | | | | | QAQTRWVQT | | | | |
| TLNGAVHLYA | 496 | 0.854 | 0.942 | 0.14 | TLNGAVHLYA | 995 | 0.743 | 0.325 | 0.09 |
| QAQVSPVQN | | | | | QAQTVQVQN | | | | |
| PLNGAVHLYA | 497 | 0.205 | 0.942 | 0.23 | ALNGAVHLYA | 996 | 0.762 | 0.324 | 0.09 |
| QAQPGWVQT | | | | | QAQKGGVQN | | | | |
| PLNGAVHLYA | 498 | 0.407 | 0.941 | 17.25 | PLNGAVHLYD | 997 | 0.143 | 0.323 | 0.09 |
| QAQSTQVQN | | | | | QAQVSGVQN | | | | |
| PLNGSVHLYA | 499 | 0.247 | 0.939 | 0.09 | PLNGAVHLYA | 998 | 0.554 | 0.323 | 0.65 |
| QAQTGWVEN | | | | | QAQTGLDQN | | | | |
| PLNGAVHLYA | 500 | 0.267 | 0.934 | 22.29 | PLNGAVPLYA | 999 | 0.387 | 0.323 | 0.42 |
| QAQQSPVQN | | | | | QAQTGWVQT | | | | |
| PLNGAVHLYA | 501 | 0.517 | 0.934 | 8.23 | PLNGAVHLYA | 1000 | 0.414 | 0.323 | 0.09 |
| QAQRYSVQN | | | | | QAQVSSVHN | | | | |
| PLNGAVHLYA | 502 | 0.267 | 0.930 | 9.77 | TLNGAVHLYA | 1001 | 0.264 | 0.322 | 0.09 |
| QAQTGWDQN | | | | | QAQTNSVQN | | | | |
| PLNGAVHLYA | 503 | 0.774 | 0.930 | 4.67 | PLNGAGHLYA | 1002 | 0.479 | 0.321 | 0.09 |
| QAQTQRVQN | | | | | QAQLSPVQN | | | | |
| ALNGAVHLYA | 504 | 0.330 | 0.926 | 0.23 | HLNGAVHLYA | 1003 | 0.532 | 0.321 | 0.19 |
| QAQTGWVRN | | | | | QAQTGRVQN | | | | |
| PLNGAVHLYA | 505 | 0.370 | 0.923 | 171 | PLNGAVHLYD | 1004 | 0.872 | 0.320 | 0.09 |
| QPQTGWVQN | | | | | QAQTAMVQN | | | | |
| ALNGAVHLYA | 506 | 0.516 | 0.920 | 0.09 | PINGAVHLYA | 1005 | 0.646 | 0.320 | 0.33 |
| QAQVSGVQN | | | | | QAQTGWVQH | | | | |
| PLNGAVHLYA | 507 | 0.813 | 0.920 | 17.01 | PLNGAVQLYA | 1006 | 0.479 | 0.319 | 0.09 |
| QAQSAGVQN | | | | | QAQTLQVQN | | | | |
| PLNGAVHLYA | 508 | 0.734 | 0.918 | 0.09 | PLNGAVHLYA | 1007 | 0.323 | 0.318 | 0.28 |
| QAQSAPVQT | | | | | QAQKGWVQT | | | | |
| PLNGAVHLYA | 509 | 0.499 | 0.914 | 13.97 | PLNGAVHHYA | 1008 | 0.592 | 0.318 | 0.09 |
| QAQRQSVQN | | | | | QAQSTSVQN | | | | |
| PLNGAVHLYA | 510 | 0.591 | 0.906 | 20.42 | PLNGYVHLYA | 1009 | 0.187 | 0.317 | 2.99 |
| QAQSQPVQN | | | | | QAQTGWVQN | | | | |
| PLNGAVHLYA | 511 | 0.570 | 0.906 | 0.09 | PLNGAVHIYA | 1010 | 0.599 | 0.316 | 0.09 |
| QAQVASVKN | | | | | QAQVQSVQN | | | | |
| PLNGAVHLYA | 512 | 0.403 | 0.904 | 0.09 | PLIGAVHLYA | 1011 | 0.535 | 0.315 | 0.09 |
| QAQTAWVRN | | | | | QAQLSPVQN | | | | |
| SLNGAVHLYA | 513 | 0.491 | 0.904 | 0.09 | TLNGAVHLYA | 1012 | 0.774 | 0.315 | 0.09 |
| QAQTMQVQN | | | | | QAQLLPVQN | | | | |
| PLNGAVNLYA | 514 | 0.664 | 0.902 | 0.09 | PLNGAVHLYS | 1013 | 0.747 | 0.315 | 0.84 |
| QAQSSPVQN | | | | | QAQTGWVQT | | | | |
| PLNGAVHLYA | 515 | 0.318 | 0.901 | 0.23 | ALNGAVHLYA | 1014 | 0.444 | 0.315 | 0.14 |
| QAQTGGGQN | | | | | QAQTQLVQN | | | | |
| PLNGAVHLYD | 516 | 0.515 | 0.898 | 0.09 | PLNGAVHLYA | 1015 | 0.810 | 0.315 | 34.86 |
| QAQVASVQN | | | | | QAQSLPVQN | | | | |
| PLNGAVHLYA | 517 | 0.709 | 0.897 | 45.94 | PVNGAVHLYA | 1016 | 0.890 | 0.314 | 0.09 |
| QAQSTSVQN | | | | | QAQTGWVPN | | | | |
| PLNGTVHLYA | 518 | 0.403 | 0.894 | 0.23 | PLNGAVHLYA | 1017 | 0.247 | 0.313 | 0.19 |
| QAQTGWVQH | | | | | QAKTVSVQN | | | | |
| QLNGAVHLYA | 519 | 0.718 | 0.892 | 0.42 | PLNGAVHLYA | 1018 | 0.645 | 0.313 | 0.09 |
| QAQTSSVQN | | | | | QAQTTSVHN | | | | |
| PLDGAVHLYA | 520 | 0.613 | 0.892 | 0.09 | PVNGAVHLYA | 1019 | 0.765 | 0.313 | 939 |
| QAQTGWVHN | | | | | QAQTGWVQN | | | | |
| PLNGTVHLYA | 521 | 0.397 | 0.888 | 0.33 | PLNGAVHLYA | 1020 | 0.476 | 0.313 | 0.09 |
| QAQTGWVQK | | | | | QAHTGWVQI | | | | |
| PLNGAVHLYA | 522 | 0.369 | 0.888 | 20.28 | PLNGAVRLYA | 1021 | 0.611 | 0.313 | 0.09 |
| QAQVLGVQN | | | | | QAQTASVQN | | | | |
| PLNGAVHLYA | 523 | 0.640 | 0.887 | 5.7 | PLNGGVHLYA | 1022 | 0.646 | 0.313 | 0.09 |
| QAQNQPVQN | | | | | QAQTGLVQK | | | | |
| PLNGAVHLYA | 524 | 0.605 | 0.885 | 21.08 | PLNGAVNLYA | 1023 | 0.652 | 0.313 | 0.14 |
| QAQLGTVQN | | | | | QAQVMSVQN | | | | |
| PLNGAVHLYA | 525 | 0.637 | 0.882 | 7.57 | PLNGAVNLYA | 1024 | 0.682 | 0.312 | 0.09 |
| QAQRAGVQN | | | | | QAQSMSVQN | | | | |
| PVNGAVHLYA | 526 | 0.837 | 0.882 | 42.25 | PLNGAVHLYA | 1025 | 0.513 | 0.312 | 0.09 |
| QAQTGLVQN | | | | | PAQTGWGQN | | | | |
| PLNGGVHLYA | 527 | 0.529 | 0.881 | 0.33 | QLNGAVHLYA | 1026 | 0.595 | 0.311 | 0.47 |
| QAQTGWVQH | | | | | QAQTGWAQN | | | | |
| PLNGAVHLYA | 528 | 0.677 | 0.879 | 116.4 | PLNGAVHLYA | 1027 | 0.451 | 0.311 | 23.13 |
| QAQTGRVQN | | | | | QAQTVWVQN | | | | |
| PLNGAVHLYA | 529 | 0.266 | 0.876 | 0.56 | QLNGAVHLYA | 1028 | 0.742 | 0.311 | 0.09 |
| QAQTGGVKN | | | | | QAQSVSVQN | | | | |
| PLNGAVHLYA | 530 | 0.251 | 0.874 | 0.14 | PLNGAVHLYA | 1029 | 0.426 | 0.311 | 0.09 |
| QAQTAWLQN | | | | | QAQTAQLQN | | | | |
| PLNGAVHLYA | 531 | 0.269 | 0.870 | 0.14 | PLNGAVHLYA | 1030 | 0.453 | 0.311 | 0.09 |
| QAQTRWVQK | | | | | QAHVASVQN | | | | |
| PLNGAVHLYA | 532 | 0.699 | 0.858 | 0.09 | PLNGAVHLYA | 1031 | 0.440 | 0.310 | 0.09 |
| QAQLAPVKN | | | | | KAQRSTVQN | | | | |
| PLNGAVHHYA | 533 | 0.390 | 0.855 | 0.09 | QLNGAVHLYA | 1032 | 0.509 | 0.310 | 1.92 |
| QAQTSSVQN | | | | | QAQTGWVKN | | | | |
| PLNGAVHLYA | 534 | 0.402 | 0.851 | 0.14 | PLNGAVHLYA | 1033 | 0.281 | 0.310 | 0.28 |
| QAQTGSVQD | | | | | QAQTRWVKN | | | | |
| PLNGAVHLYA | 535 | 0.557 | 0.849 | 27.67 | PLNGAVHLYA | 1034 | 0.381 | 0.310 | 0.19 |
| QAQTGMVQN | | | | | QAQTAWIQN | | | | |
| PLNGAVHLYA | 536 | 0.647 | 0.848 | 15.14 | PLNGAVHLYA | 1035 | 0.393 | 0.310 | 0.09 |
| QAQLSNVQN | | | | | QAQLSQVKN | | | | |
| PLNGAVHLYA | 537 | 0.502 | 0.844 | 14.39 | SLNGAVHLYA | 1036 | 0.816 | 0.310 | 2284 |
| QAQRLPVQN | | | | | QAQTGWVQN | | | | |
| PLNGAVHLYA | 538 | 0.782 | 0.842 | 7.52 | PLNGAVHLYA | 1037 | 0.390 | 0.309 | 0.14 |
| QAQRQGVQN | | | | | QAQTSTVKN | | | | |
| PLNGAVHLYA | 539 | 0.563 | 0.839 | 0.09 | PLNGAVHLYA | 1038 | 0.654 | 0.309 | 16.82 |
| QAQSTPVQT | | | | | QAQALPVQN | | | | |
| QLNGNVHLYA | 540 | 0.844 | 0.838 | 5.56 | PLNGAVHLYD | 1039 | 0.158 | 0.309 | 0.33 |
| QAQTGWVQN | | | | | QAQTGWFQN | | | | |
| PLNGGVHLYA | 541 | 0.559 | 0.835 | 0.84 | PLNGAVHVYA | 1040 | 0.527 | 0.309 | 0.09 |
| QAQTGWVQT | | | | | QAQTGWFQN | | | | |
| PLNGAVHLYA | 542 | 0.470 | 0.833 | 0.09 | PINGAVHLYA | 1041 | 0.386 | 0.308 | 0.09 |
| QAQTTPVKN | | | | | QAQVGSVQN | | | | |
| PLNGAVHLYA | 543 | 0.700 | 0.832 | 18.65 | PLNGSVHLYA | 1042 | 0.418 | 0.308 | 0.14 |
| QAQRLTVQN | | | | | QAQTTGVQN | | | | |
| PLNGAVHLYA | 544 | 0.373 | 0.830 | 7.5 | PLNGAVHLYA | 1043 | 0.874 | 0.307 | 0.09 |
| QAQTLRVQN | | | | | KAQASPVQN | | | | |
| PLNGAVHLYA | 545 | 0.742 | 0.830 | 7.34 | PLNGSVHLYA | 1044 | 0.192 | 0.307 | 0.09 |
| QAQSAFVQN | | | | | QAQKAWVQN | | | | |
| SLNGAVHLYA | 546 | 0.586 | 0.824 | 0.09 | SLNGAVHLYA | 1045 | 0.270 | 0.307 | 0.09 |
| QAQTNSVQN | | | | | QAQVLGVQN | | | | |
| PLNGAVHLYA | 547 | 0.658 | 0.824 | 6.92 | PLNGAVHLYA | 1046 | 0.827 | 0.306 | 0.09 |
| QAQSVQVQN | | | | | QEQLMPVQN | | | | |
| PLDGAVHLYA | 548 | 0.568 | 0.823 | 0.09 | ALNGAVHLYA | 1047 | 0.872 | 0.306 | 18.51 |
| QAQTAAVQN | | | | | QSQTGWVQN | | | | |
| SLNGAVHLYA | 549 | 0.532 | 0.821 | 0.09 | PLNGYVHLYD | 1048 | 0.508 | 0.305 | 0.09 |
| QAQLMPVQN | | | | | QAQTGWVQN | | | | |
| PLDGAVHLYA | 550 | 0.621 | 0.820 | 0.47 | PLNGAVHLYA | 1049 | 0.505 | 0.305 | 0.09 |
| QAQTGWVQT | | | | | QAQTSMVQT | | | | |
| PLNGAVHLYA | 551 | 0.672 | 0.819 | 49.77 | QLNGAVHLYA | 1050 | 0.588 | 0.304 | 0.14 |
| QAQLLPVQN | | | | | QAQISSVQN | | | | |
| PLNGAVHLYA | 552 | 0.738 | 0.815 | 6.64 | PVNGAVHLYA | 1051 | 0.723 | 0.304 | 1.12 |
| QAQRTQVQN | | | | | QAQTGWVQT | | | | |
| PLNGAVHLYA | 553 | 0.254 | 0.813 | 0.79 | PLNGAVHLYA | 1052 | 0.713 | 0.303 | 0.09 |
| QAQTGSDQN | | | | | QEQTATVQN | | | | |
| PLNGAVHLYA | 554 | 0.430 | 0.808 | 0.09 | PLNGAVYLYA | 1053 | 0.352 | 0.302 | 0.09 |
| QAQVASDQN | | | | | QAQTVSVQN | | | | |
| PLNGAVNLYA | 555 | 0.744 | 0.805 | 0.09 | PLNGNVHLYA | 1054 | 0.521 | 0.302 | 0.09 |
| QAQLAPVQN | | | | | QAQTASVQN | | | | |
| PLNGSVHLYA | 556 | 0.160 | 0.804 | 0.37 | PLNGYVHLYA | 1055 | 0.556 | 0.301 | 0.09 |
| QAQTGWLQN | | | | | QAQTGLVQN | | | | |
| PLNGTVHLYA | 557 | 0.253 | 0.801 | 500.6 | PLNGAVHLYD | 1056 | 0.402 | 0.301 | 0.14 |
| QAQTGWVQN | | | | | QAQTLSVQN | | | | |
| PLNGAVHLYA | 558 | 0.857 | 0.801 | 0.14 | PLNGAVHLYA | 1057 | 0.499 | 0.300 | 0.14 |
| QAQVSPVKN | | | | | QAQTSSVQH | | | | |
| PLNGAVHLYA | 559 | 0.480 | 0.801 | 1.22 | SLNGAVHLYA | 1058 | 0.737 | 0.300 | 0.19 |
| QAQLPPVQN | | | | | QAQTGWVPN | | | | |
| PLNGAVHLYA | 560 | 0.521 | 0.794 | 0.14 | PLNGAVHLYA | 1059 | 0.839 | 0.300 | 0.09 |
| QAQSSPVQT | | | | | QAQTAMVKN | | | | |
| PLNGAVHLYA | 561 | 0.585 | 0.790 | 8.88 | TLNGAVHLYA | 1060 | 0.700 | 0.299 | 0.14 |
| QAQAQPVQN | | | | | QAQRLSVQN | | | | |
| PLNGDVHLYA | 562 | 0.434 | 0.789 | 0.09 | ALNGAVHLYA | 1061 | 0.341 | 0.297 | 1.64 |
| QAQTTSVQN | | | | | QAQTGWVQK | | | | |
| PLNGAVHLYA | 563 | 0.392 | 0.788 | 0.75 | PLNGAVHLYA | 1062 | 0.722 | 0.297 | 0.19 |
| QAQTPQVQN | | | | | KAQTALVQN | | | | |
| PLNGAVHLYA | 564 | 0.571 | 0.787 | 29.35 | QLNGAVHLYA | 1063 | 0.743 | 0.297 | 0.23 |
| QAQTSWVQN | | | | | QAQTAKVQN | | | | |
| PLNGAVHLYA | 565 | 0.254 | 0.784 | 0.51 | PLNGADHLYA | 1064 | 0.744 | 0.297 | 0.14 |
| QAQTGGDQN | | | | | QAQTTSVQN | | | | |
| PLNGAVHLYA | 566 | 0.547 | 0.782 | 0.14 | PLNGAVHLYA | 1065 | 0.259 | 0.297 | 0.93 |
| QAQLSPVKN | | | | | QAQTPWVQN | | | | |
| PLNGAVHLYA | 567 | 0.603 | 0.782 | 0.09 | PLNGTVHLYA | 1066 | 0.324 | 0.296 | 0.28 |
| QAQSSPVKN | | | | | QAQTGWIQN | | | | |
| PLNGAVHLYA | 568 | 0.727 | 0.781 | 8.41 | PLNGAVHLYA | 1067 | 0.286 | 0.296 | 0.23 |
| QAQNTTVQN | | | | | QAHTGWVQH | | | | |
| PLDGAVHLYA | 569 | 0.716 | 0.780 | 0.19 | PLNGAVHLYA | 1068 | 0.622 | 0.296 | 0.09 |
| QAQTGWVQH | | | | | RAQTASVQN | | | | |
| PLNGAVHLYA | 570 | 0.823 | 0.773 | 6.31 | TLNGAVHLYA | 1069 | 0.876 | 0.295 | 0.14 |
| QAQTTRVQN | | | | | QAQRSLVQN | | | | |
| PLDGAVHLYA | 571 | 0.722 | 0.767 | 0.09 | PLNGAVHLYA | 1070 | 0.481 | 0.294 | 0.14 |
| QAQTGWLQN | | | | | QAHTGWVQK | | | | |
| PLNGSVHLYA | 572 | 0.681 | 0.757 | 8.32 | PLNGDVHLYA | 1071 | 0.859 | 0.294 | 0.14 |
| QPQTGWVQN | | | | | QAQTAQVQN | | | | |
| PLNGAVHLYS | 573 | 0.794 | 0.755 | 25.1 | TLNGAVHLYA | 1072 | 0.407 | 0.294 | 0.14 |
| QAQTGLVQN | | | | | QAQTQPVQN | | | | |
| PLNGAVHLYA | 574 | 0.654 | 0.755 | 14.16 | PLNGAVHLYA | 1073 | 0.505 | 0.294 | 0.7 |
| QAQTQWVQN | | | | | QAQTPRVQN | | | | |
| PLNGSVHLYA | 575 | 0.187 | 0.754 | 0.33 | PRNGAVHLYA | 1074 | 0.200 | 0.293 | 0.09 |
| QAQTGWVQI | | | | | QAQTMSVQN | | | | |
| PLNGAVHLYA | 576 | 0.259 | 0.753 | 0.28 | PLNGAVHLYA | 1075 | 0.118 | 0.293 | 0.23 |
| QAQTGSVHN | | | | | QAHTVSVQN | | | | |
| ALNGAVHLYA | 577 | 0.367 | 0.751 | 1129 | PVNGAVHLYA | 1076 | 0.823 | 0.293 | 0.14 |
| QAQTGWVQN | | | | | QAQTGLVQT | | | | |
| PLNGTVHLYA | 578 | 0.224 | 0.749 | 0.14 | PLNGAVHLYA | 1077 | 0.899 | 0.292 | 87.25 |
| QAQTGWVRN | | | | | QAQSSSVQN | | | | |
| PLNGAVHLYA | 579 | 0.240 | 0.749 | 0.23 | PLNGAVHLYA | 1078 | 0.497 | 0.292 | 0.84 |
| QAQTGGLQN | | | | | QAQRPPVQN | | | | |
| SLNGAVHLYA | 580 | 0.712 | 0.748 | 0.09 | PLNGAVHLYT | 1079 | 0.779 | 0.291 | 0.09 |
| QAQVSGVQN | | | | | QAQVSPVQN | | | | |
| PLNGAVHLYA | 581 | 0.404 | 0.747 | 0.37 | PLNGAVPLYA | 1080 | 0.661 | 0.291 | 0.09 |
| QAQTAWVQK | | | | | QAQTTPVQN | | | | |
| PLNGAVHLYA | 582 | 0.660 | 0.747 | 0.19 | ALNGAVHLYA | 1081 | 0.293 | 0.290 | 1.59 |
| QAQTGRVQT | | | | | QAQTGWVKN | | | | |
| TLNGAVHLYA | 583 | 0.754 | 0.739 | 0.09 | SLNGAVHLYA | 1082 | 0.871 | 0.290 | 0.56 |
| QAQSAPVQN | | | | | QAQTGWVRN | | | | |
| PLNGAVHLYD | 584 | 0.449 | 0.738 | 0.09 | PLNGAVHLYA | 1083 | 0.316 | 0.288 | 0.09 |
| QAQTVAVQN | | | | | QAQLAGVKN | | | | |
| QLNGAVHLYA | 585 | 0.892 | 0.735 | 48.7 | PLNGAVHLYA | 1084 | 0.427 | 0.288 | 0.09 |
| QAQTGSVQN | | | | | QAQTSPAQN | | | | |
| PLNGAVHLYA | 586 | 0.858 | 0.733 | 5.37 | PLNGAVHLYA | 1085 | 0.539 | 0.288 | 0.19 |
| QAQNTQVQN | | | | | KAQLSSVQN | | | | |
| PLNGAVHLYA | 587 | 0.512 | 0.729 | 5.05 | PLNGAVHLYA | 1086 | 0.526 | 0.285 | 0.14 |
| QAQTGWLQN | | | | | QAQRSPVQT | | | | |
| PINGAVHLYA | 588 | 0.684 | 0.721 | 45.57 | PLNGAVHLYA | 1087 | 0.826 | 0.285 | 0.14 |
| QAQTGLVQN | | | | | HAQTMSVQN | | | | |
| PLDGAVHLYA | 589 | 0.600 | 0.719 | 0.09 | PLNGNVHLYA | 1088 | 0.429 | 0.284 | 0.14 |
| QAQTGWVQI | | | | | QAQTGWVKN | | | | |
| PLNGSVHLYA | 590 | 0.369 | 0.713 | 0.09 | PLNGAVHLYA | 1089 | 0.409 | 0.284 | 0.09 |
| QAQTGSVQT | | | | | QAQTGWVPH | | | | |
| PLNGAVQLYA | 591 | 0.460 | 0.711 | 0.09 | PLNGAVHLYA | 1090 | 0.442 | 0.284 | 0.09 |
| QAQTSSVQN | | | | | QAQPGWGQN | | | | |
| PLNGAVHLYA | 592 | 0.658 | 0.707 | 5.61 | PLNGVVHLYA | 1091 | 0.291 | 0.284 | 0.14 |
| QAQTYAVQN | | | | | QAQTVSVQN | | | | |
| PLNGAVHLYA | 593 | 0.575 | 0.703 | 81.37 | PLNGDVHLYA | 1092 | 0.499 | 0.283 | 0.19 |
| QAQTLSVQN | | | | | QAQTSSVQN | | | | |
| PLNGAVHLYA | 594 | 0.874 | 0.703 | 12.15 | PLNGAVHIYA | 1093 | 0.570 | 0.283 | 0.14 |
| QAQNLPVQN | | | | | QAQTLSVQN | | | | |
| PLNGAVHLYA | 595 | 0.328 | 0.702 | 0.09 | PLNGAVHLYA | 1094 | 0.597 | 0.283 | 0.19 |
| QAQTSSDQN | | | | | QAQIPPVQN | | | | |
| PLNGAVHLYA | 596 | 0.736 | 0.702 | 23.93 | SLNGAVHLYA | 1095 | 0.856 | 0.282 | 1.03 |
| QAQATSVQN | | | | | QAQTGWVQH | | | | |
| TLNGAVHLYA | 597 | 0.353 | 0.701 | 0.23 | PLNAAVHLYA | 1096 | 0.466 | 0.282 | 0.14 |
| QAQLSPVQN | | | | | QAQTGWDQN | | | | |
| PLNGAVHLYA | 598 | 0.271 | 0.700 | 0.14 | PLNGAVHRYA | 1097 | 0.823 | 0.282 | 0.09 |
| QAQTAWFQN | | | | | QAQTTSVQN | | | | |
| TLNGAVHLYA | 599 | 0.801 | 0.700 | 0.09 | PLNGSVHLYA | 1098 | 0.394 | 0.281 | 0.09 |
| QAQVTPVQN | | | | | QAQPGSVQN | | | | |
| TLNGAVHLYA | 600 | 0.202 | 0.696 | 0.09 | PLDGAVHLYA | 1099 | 0.838 | 0.281 | 0.09 |
| QAQTAAVQN | | | | | QAQTGWVEN | | | | |
| PLNGAVHLYA | 601 | 0.801 | 0.693 | 7.76 | PLNGAVHLYA | 1100 | 0.524 | 0.281 | 0.33 |
| QAQATQVQN | | | | | QAQTGRVKN | | | | |
| RLDGAVHLYA | 602 | 0.861 | 0.693 | 8.37 | PLNGAVHLYA | 1101 | 0.435 | 0.281 | 0.33 |
| QAQTGWVQN | | | | | QAQTGRLQN | | | | |
| QLNGAVHLYA | 603 | 0.840 | 0.691 | 0.09 | PLNGSVHLYA | 1102 | 0.584 | 0.281 | 0.28 |
| QAQTGWVEN | | | | | QAQTARVQN | | | | |
| PLNGAVHLYA | 604 | 0.278 | 0.691 | 0.19 | PLNGAVHLYD | 1103 | 0.378 | 0.280 | 0.19 |
| QAQVSSVKN | | | | | QAQLSSVQN | | | | |
| PLNGAVHLYA | 605 | 0.582 | 0.690 | 94.87 | ALNGAVHLYA | 1104 | 0.550 | 0.280 | 0.09 |
| QAQLSSVQN | | | | | QAQTRLVQN | | | | |
| PLNGAVHLYA | 606 | 0.209 | 0.689 | 0.33 | PLNGAVNLYA | 1105 | 0.780 | 0.280 | 0.19 |
| QAQTGSGQN | | | | | QAQTAQVQN | | | | |
| PLNGAVHLYA | 607 | 0.441 | 0.686 | 0.09 | PLNGAVHLYA | 1106 | 0.461 | 0.279 | 0.09 |
| QAQLQPVQT | | | | | QAQLSSVQH | | | | |
| PINGAVHLYA | 608 | 0.805 | 0.686 | 0.09 | PLNGAVHLYA | 1107 | 0.799 | 0.279 | 0.09 |
| QAQLSSVQN | | | | | QAHTGPVQN | | | | |
| PINGAVHLYA | 609 | 0.313 | 0.684 | 0.09 | PLKGAVHLYA | 1108 | 0.872 | 0.279 | 0.09 |
| QAQTVAVQN | | | | | QAQTLSVQN | | | | |
| PLNGAVHLYA | 610 | 0.546 | 0.680 | 0.09 | ALNGAVHLYA | 1109 | 0.499 | 0.279 | 0.09 |
| QAQVSAVKN | | | | | QAQKAWVQN | | | | |
| SLNGAVHLYA | 611 | 0.461 | 0.680 | 0.23 | PLNGAVHLYA | 1110 | 0.386 | 0.278 | 0.09 |
| QAQTVAVQN | | | | | QAQAGWVQT | | | | |
| PLNGAVHLYA | 612 | 0.882 | 0.680 | 22.29 | PLNGAVHLYA | 1111 | 0.709 | 0.278 | 0.19 |
| QAQTQAVQN | | | | | QAQTGLVQS | | | | |
| QLNGAVHLYA | 613 | 0.398 | 0.679 | 0.28 | PLNGAVHLYA | 1112 | 0.436 | 0.277 | 0.14 |
| QAQTAQVQN | | | | | QAQTGCVQI | | | | |
| PLNGAVHLYD | 614 | 0.309 | 0.676 | 0.14 | QLNGAVHLYA | 1113 | 0.529 | 0.276 | 1.26 |
| QAQTMSVQN | | | | | QAQPGWVQN | | | | |
| PLNGAVHLYA | 615 | 0.341 | 0.675 | 0.19 | PLNGAVHLYA | 1114 | 0.678 | 0.276 | 0.37 |
| QPQTGWVQK | | | | | QAQRPGVQN | | | | |
| PLNGAVHLYA | 616 | 0.855 | 0.672 | 30.47 | PLNGAVHLYA | 1115 | 0.265 | 0.276 | 0.09 |
| QAQVMPVQN | | | | | QAQTAAVQH | | | | |
| PLNGAVHLYA | 617 | 0.208 | 0.669 | 0.19 | PLNGADHLYA | 1116 | 0.728 | 0.276 | 0.09 |
| QAQTVSVQK | | | | | QAQTMPVQN | | | | |
| PLNGAVHLYA | 618 | 0.410 | 0.664 | 0.09 | PLNGAVHLYA | 1117 | 0.198 | 0.276 | 0.33 |
| QAQTAWAQN | | | | | KAQTGWVQT | | | | |
| PLNGAVHLYA | 619 | 0.764 | 0.664 | 12.06 | QLNGAVHLYA | 1118 | 0.819 | 0.275 | 0.09 |
| QAQNALVQN | | | | | QAQKAWVQN | | | | |
| TLNGAVHLYA | 620 | 0.217 | 0.663 | 0.09 | PLNGAVHLYA | 1119 | 0.116 | 0.274 | 0.98 |
| QAQVSAVQN | | | | | QAQTGCDQN | | | | |
| PLKGAVHLYA | 621 | 0.611 | 0.663 | 0.09 | PLNGTVHLYA | 1120 | 0.513 | 0.274 | 0.09 |
| QAQTTPVQN | | | | | QEQTGSVQN | | | | |
| QLNGSVHLYA | 622 | 0.776 | 0.657 | 96.83 | PLNGAVNLYA | 1121 | 0.671 | 0.274 | 0.09 |
| QAQTGWVQN | | | | | QAQSSLVQN | | | | |
| SLNGAVHLYA | 623 | 0.307 | 0.650 | 0.09 | ALNGAVHLYA | 1122 | 0.296 | 0.274 | 0,23 |
| QAQVATVQN | | | | | QAQTGWVEN | | | | |
| PLNGAVHLYA | 624 | 0.876 | 0.650 | 9.39 | PLNGTVHLYA | 1123 | 0.405 | 0.274 | 0.14 |
| QAQRMLVQN | | | | | QAQTGWVQY | | | | |
| PLNGAVHLYA | 625 | 0.589 | 0.649 | 0.09 | PINGAVHLYA | 1124 | 0.373 | 0.273 | 0.75 |
| QAQTVAVKN | | | | | QAQTGWVQK | | | | |
| PLNGAVHLYD | 626 | 0.523 | 0.649 | 0.14 | PLNGAVHLYA | 1125 | 0.820 | 0.273 | 0.09 |
| QAQTTSVQN | | | | | QAQTGRVRN | | | | |
| PLNGAVHLYA | 627 | 0.725 | 0.647 | 11.92 | PLNGAVHLYA | 1126 | 0.590 | 0.272 | 0.37 |
| QAQRQLVQN | | | | | QAQTGRDQN | | | | |
| PLNGAVHLYA | 628 | 0.754 | 0.645 | 28.79 | QLNGAVHLYA | 1127 | 0.409 | 0.272 | 0.23 |
| QAQTLGVQN | | | | | QAQSALVQN | | | | |
| PLNGAVHLYA | 629 | 0.889 | 0.645 | 0.09 | PLNGVVHLYA | 1128 | 0.422 | 0.272 | 0.09 |
| QAQLGPVQT | | | | | QAQTGWVQK | | | | |
| PLNGGVHLYA | 630 | 0.554 | 0.644 | 0.14 | PLNGAVHLYA | 1129 | 0.534 | 0.272 | 0.14 |
| QAQTGWVQD | | | | | QAHTGWGQN | | | | |
| PLNGAVHLYA | 631 | 0.205 | 0.640 | 0.98 | PLNGDVHLYA | 1130 | 0.638 | 0.272 | 0.09 |
| QAQTGSVKN | | | | | QAQRSLVQN | | | | |
| PLNGAVNLYA | 632 | 0.332 | 0.635 | 0.09 | PLNGNVHLYA | 1131 | 0.711 | 0.272 | 0.09 |
| QAQLASVQN | | | | | QAQTVWVQN | | | | |
| PLNGAVHLYD | 633 | 0.307 | 0.634 | 0.14 | PLNGAVHLYA | 1132 | 0.332 | 0.272 | 0.23 |
| QAQVSSVQN | | | | | QAPTGWVQH | | | | |
| PLNGAVHLYA | 634 | 0.355 | 0.632 | 0.14 | PLNGAVHLYS | 1133 | 0.725 | 0.271 | 0.23 |
| QAQTMSDQN | | | | | QAQTGWGQN | | | | |
| PLNGAVHLYA | 635 | 0.337 | 0.630 | 17.48 | ALNGSVHLYA | 1134 | 0.729 | 0.271 | 0.09 |
| QAQTGWVQK | | | | | QAQTGWVQT | | | | |
| PLNGAVHLYA | 636 | 0.203 | 0.630 | 0.09 | PLNGAVQLYA | 1135 | 0.347 | 0.271 | 0.09 |
| QAQLASVKN | | | | | QAQRSTVQN | | | | |
| PLNGGVHLYA | 637 | 0.490 | 0.628 | 0.23 | PLNGTVHLYA | 1136 | 0.434 | 0.271 | 0.28 |
| QAQTGWGQN | | | | | QAQTGTVQN | | | | |
| QLNGSVHLYA | 638 | 0.844 | 0.627 | 0.09 | PLNGTVHLYA | 1137 | 0.549 | 0.271 | 0.37 |
| QAQTGWVQT | | | | | QAQTGWVQI | | | | |

TABLE 20
NGS fold-enrichment of TTD-001 matured AAV capsid variants brain and DRG of
cynomolgus macaques and the brain of marmosets

| | | Brain of cynomolgus | | DRG of cynomolgus | | | |
|---|---|---|---|---|---|---|---|
| | | macaques | | macaques | | Brain of marmosets | |
| | | Fold | Fold | Fold | Fold | Fold | Fold |
| | SEQ | change | change | change | change | change | change |
| | ID | relative | relative | relative | relative | relative | relative |
| Sequence | NO | to AAV9 | to TTD-001 | to AAV9 | to TTD-001 | to AAV9 | to TTD-001 |
| ALNGAVHLYAQAQ | 1122 | 12.707 | 0.274 | 0.000 | 0.000 | 420.579 | 571.155 |
| TGWVEN | | | | | | | |
| PINGAVHLYAQAQ | 40 | 8.716 | 0.188 | 2.100 | 13.718 | 573.779 | 779.203 |
| TGWVEN | | | | | | | |
| PLNGAVHLNAQAQ | 41 | 0.919 | 0.020 | 2.175 | 14.212 | 677.878 | 920.572 |
| TGWVEN | | | | | | | |
| PLNGAVHLYAQAQ | 42 | 5.505 | 0.119 | 0.000 | 0.000 | 263.098 | 357.292 |
| SGWVEN | | | | | | | |
| PLNGAVHLYAQAQ | 43 | 8.721 | 0.188 | 0.827 | 5.405 | 236.026 | 320.528 |
| TAWVEN | | | | | | | |
| PLNGAVHLYAQAQ | 44 | 4.126 | 0.089 | 0.245 | 1.603 | 475.164 | 645.282 |
| TGCVEN | | | | | | | |
| PLNGAVHLYAQAQ | 871 | 18.851 | 0.406 | 0.491 | 3.206 | 288.900 | 392.332 |
| TGGVEN | | | | | | | |
| PLNGAVHLYAQAQ | 45 | 5.050 | 0.109 | 0.000 | 0.000 | 541.666 | 735.593 |
| TGLVEN | | | | | | | |
| PLNGAVHLYAQAQ | 818 | 20.878 | 0.450 | 2.856 | 18.662 | 571.096 | 775.559 |
| TGSVEN | | | | | | | |
| PLNGAVHLYAQAQ | 804 | 21.437 | 0.462 | 1.362 | 8.901 | 381.679 | 518.327 |
| TGWVEN | | | | | | | |
| PLNGSVHLYAQAQ | 499 | 43.595 | 0.939 | 2.334 | 15.248 | 490.137 | 665.615 |
| TGWVEN | | | | | | | |
| PVNGAVHLYAQAQ | 46 | 6.311 | 0.136 | 0.000 | 0.000 | 506.376 | 687.669 |
| TGWVEN | | | | | | | |
| QLNGAVHLYAQAQ | 603 | 32.086 | 0.691 | 0.000 | 0.000 | 290.854 | 394.985 |
| TGWVEN | | | | | | | |
| SLNGAVHLYAQAQ | 47 | 8.707 | 0.188 | 2.111 | 13.795 | 302.059 | 410.202 |
| TGWVEN | | | | | | | |
| TLNGAVHLYAQAQ | 48 | 0.707 | 0.015 | 0.624 | 4.074 | 254.330 | 345.385 |
| TGWVEN | | | | | | | |
| ALNGAVHLYAQAQ | 819 | 20.878 | 0.450 | 0.667 | 4.356 | 232.682 | 315.986 |
| TGWVHN | | | | | | | |
| PHNGAVHLYAQAQ | 49 | 3.604 | 0.078 | 4.410 | 28.816 | 177.855 | 241.530 |
| TGWVHN | | | | | | | |
| PINGAVHLYAQAQ | 50 | 6.387 | 0.138 | 1.083 | 7.076 | 124.477 | 169.043 |
| TGWVHN | | | | | | | |
| PLKGAVHLYAQAQ | 51 | 0.986 | 0.021 | 0.516 | 3.368 | 118.166 | 160.472 |
| TGWVHN | | | | | | | |
| PLNGAAHLYAQAQ | 52 | 0.757 | 0.016 | 1.024 | 6.693 | 91.230 | 123.892 |
| TGWVHN | | | | | | | |
| PLNGADHLYAQAQ | 53 | 0.842 | 0.018 | 0.540 | 3.529 | 15.514 | 21.069 |
| TGWVHN | | | | | | | |
| PLNGAGHLYAQAQ | 54 | 1.198 | 0.026 | 1.451 | 9.479 | 106.338 | 144.409 |
| TGWVHN | | | | | | | |
| PLNGALHLYAQAQ | 55 | 2.023 | 0.044 | 0.000 | 0.000 | 148.895 | 202.202 |
| TGWVHN | | | | | | | |
| PLNGAVDLYAQAQ | 56 | 3.104 | 0.067 | 0.000 | 0.000 | 149.296 | 202.747 |
| TGWVHN | | | | | | | |
| PLNGAVHHYAQAQ | 57 | 2.959 | 0.064 | 1.184 | 7.735 | 162.209 | 220.283 |
| TGWVHN | | | | | | | |
| PLNGAVHIYAQAQ | 58 | 2.284 | 0.049 | 0.000 | 0.000 | 160.204 | 217.560 |
| TGWVHN | | | | | | | |
| PLNGAVHLDAQAQ | 59 | 0.667 | 0.014 | 0.333 | 2.175 | 298.308 | 405.108 |
| TGWVHN | | | | | | | |
| PLNGAVHLNAQAQ | 60 | 2.297 | 0.050 | 2.821 | 18.432 | 417.906 | 567.525 |
| TGWVHN | | | | | | | |
| PLNGAVHLSAQAQ | 61 | 1.946 | 0.042 | 1.987 | 12.982 | 427.101 | 580.011 |
| TGWVHN | | | | | | | |
| PLNGAVHLYAKAQ | 62 | 7.514 | 0.162 | 0.000 | 0.000 | 75.629 | 102.705 |
| TGWVHN | | | | | | | |
| PLNGAVHLYAQAK | 63 | 2.441 | 0.053 | 0.998 | 6.522 | 78.923 | 107.179 |
| TGWVHN | | | | | | | |
| PLNGAVHLYAQAQ | 64 | 1.527 | 0.033 | 0.000 | 0.000 | 132.644 | 180.133 |
| AGWVHN | | | | | | | |
| PLNGAVHLYAQAQ | 65 | 4.707 | 0.101 | 0.000 | 0.000 | 24.765 | 33.632 |
| RGWVHN | | | | | | | |
| PLNGAVHLYAQAQ | 66 | 3.622 | 0.078 | 0.000 | 0.000 | 117.858 | 160.053 |
| SGWVHN | | | | | | | |
| PLNGAVHLYAQAQ | 946 | 16.279 | 0.351 | 0.452 | 2.953 | 234.444 | 318.380 |
| TAWVHN | | | | | | | |
| PLNGAVHLYAQAQ | 67 | 4.347 | 0.094 | 0.356 | 2.328 | 255.533 | 347.018 |
| TGCVHN | | | | | | | |
| PLNGAVHLYAQAQ | 491 | 44.261 | 0.954 | 1.261 | 8.238 | 175.856 | 238.815 |
| TGGVHN | | | | | | | |
| PLNGAVHLYAQAQ | 68 | 6.977 | 0.150 | 0.000 | 0.000 | 140.966 | 191.435 |
| TGLVHN | | | | | | | |
| PLNGAVHLYAQAQ | 576 | 34.946 | 0.753 | 2.147 | 14.025 | 187.304 | 254.363 |
| TGSVHN | | | | | | | |
| PLNGAVHLYAQAQ | 755 | 23.437 | 0.505 | 0.865 | 5.655 | 192.126 | 260.911 |
| TGWVHN | | | | | | | |
| PLNGAVHLYAQAQ | 69 | 8.212 | 0.177 | 0.000 | 0.000 | 319.448 | 433.817 |
| VSAVHN | | | | | | | |
| PLNGAVHLYAQEQ | 70 | 2.986 | 0.064 | 0.000 | 0.000 | 93.114 | 126.451 |
| TGWVHN | | | | | | | |
| PLNGAVHLYAQGQ | 71 | 1.725 | 0.037 | 0.000 | 0.000 | 26.926 | 36.566 |
| TGWVHN | | | | | | | |
| PLNGAVHLYDQAQ | 72 | 1.514 | 0.033 | 0.279 | 1.822 | 142.794 | 193.917 |
| TGWVHN | | | | | | | |
| PLNGAVHLYGQAQ | 73 | 1.401 | 0.030 | 0.000 | 0.000 | 116.707 | 158.491 |
| TGWVHN | | | | | | | |
| PLNGAVHLYSQAQ | 74 | 9.806 | 0.211 | 0.000 | 0.000 | 100.716 | 136.775 |
| TGWVHN | | | | | | | |
| PLNGAVHRYAQAQ | 75 | 5.991 | 0.129 | 0.000 | 0.000 | 13.869 | 18.834 |
| TGWVHN | | | | | | | |
| PLNGAVHVYAQAQ | 76 | 2.959 | 0.064 | 0.000 | 0.000 | 159.742 | 216.933 |
| TGWVHN | | | | | | | |
| PLNGAVNLYAQAQ | 77 | 2.387 | 0.051 | 0.631 | 4.121 | 78.989 | 107.268 |
| TGWVHN | | | | | | | |
| PLNGAVPLYAQAQ | 78 | 2.946 | 0.063 | 0.774 | 5.060 | 124.967 | 169.708 |
| TGWVHN | | | | | | | |
| PLNGAVQLYAQAQ | 79 | 2.338 | 0.050 | 1.085 | 7.091 | 105.426 | 143.170 |
| TGWVHN | | | | | | | |
| PLNGGVHLYAQAQ | 979 | 15.473 | 0.333 | 0.626 | 4.090 | 185.552 | 251.984 |
| TGWVHN | | | | | | | |
| PLNGSVHLYAQAQ | 703 | 25.824 | 0.556 | 1.283 | 8.381 | 360.426 | 489.465 |
| TGWVHN | | | | | | | |
| PLNGTVHLYAQAQ | 80 | 10.315 | 0.222 | 0.000 | 0.000 | 159.224 | 216.229 |
| TGWVHN | | | | | | | |
| PRNGAVHLYAQAQ | 81 | 2.842 | 0.061 | 1.016 | 6.636 | 163.614 | 222.192 |
| TGWVHN | | | | | | | |
| PVNGAVHLYAQAQ | 82 | 8.023 | 0.173 | 0.761 | 4.971 | 402.937 | 547.197 |
| TGWVHN | | | | | | | |
| QLNGAVHLYAQAQ | 816 | 21.018 | 0.453 | 0.794 | 5.185 | 344.539 | 467.890 |
| TGWVHN | | | | | | | |
| SLNGAVHLYAQAQ | 83 | 8.378 | 0.181 | 1.286 | 8.399 | 211.470 | 287.181 |
| TGWVHN | | | | | | | |
| TLNGAVHLYAQAQ | 84 | 1.270 | 0.027 | 0.252 | 1.648 | 136.758 | 185.720 |
| TGWVHN | | | | | | | |
| ALNGAVHLYAQAQ | 1081 | 13.477 | 0.290 | 0.084 | 0.546 | 9.629 | 13.077 |
| TGWVKN | | | | | | | |
| ALNGAVNLYAQAQ | 85 | 4.203 | 0.091 | 0.000 | 0.000 | 3.919 | 5.323 |
| TGWVKN | | | | | | | |
| KLNGAVHLYAQAQ | 86 | 7.153 | 0.154 | 0.000 | 0.000 | 9.576 | 13.004 |
| TGWVKN | | | | | | | |
| PHNGAVHLYAQAQ | 87 | 1.248 | 0.027 | 0.378 | 2.467 | 5.661 | 7.688 |
| TGWVKN | | | | | | | |
| PINGAVHLYAQAQ | 88 | 7.644 | 0.165 | 0.143 | 0.933 | 7.560 | 10.267 |
| TGWVKN | | | | | | | |
| PLKGAVHLYAQAQ | 89 | 0.748 | 0.016 | 0.103 | 0.675 | 7.088 | 9.626 |
| TGWVKN | | | | | | | |
| PLNAAVHLYAQAQ | 90 | 2.473 | 0.053 | 0.000 | 0.000 | 18.662 | 25.343 |
| TGWVKN | | | | | | | |
| PLNGAAHLYAQAQ | 91 | 0.901 | 0.019 | 0.390 | 2.547 | 5.894 | 8.004 |
| TGWVKN | | | | | | | |
| PLNGADHLYAQAQ | 92 | 3.270 | 0.070 | 0.316 | 2.064 | 0.970 | 1.317 |
| TGWVKN | | | | | | | |
| PLNGAGHLYAQAQ | 93 | 0.378 | 0.008 | 0.209 | 1.367 | 11.306 | 15.354 |
| TGWVKN | | | | | | | |
| PLNGALHLYAQAQ | 859 | 19.306 | 0.416 | 0.059 | 0.387 | 19.870 | 26.984 |
| TGWVKN | | | | | | | |
| PLNGAVDLYAQAQ | 94 | 0.937 | 0.020 | 0.000 | 0.000 | 5.016 | 6.812 |
| TGWVKN | | | | | | | |
| PLNGAVHHYAQAQ | 95 | 1.572 | 0.034 | 0.122 | 0.800 | 8.227 | 11.173 |
| TGWVKN | | | | | | | |
| PLNGAVHIYAQAQ | 96 | 11.752 | 0.253 | 0.310 | 2.024 | 10.015 | 13.600 |
| TGWVKN | | | | | | | |
| PLNGAVHLDAQAQ | 97 | 0.455 | 0.010 | 0.132 | 0.864 | 14.604 | 19.832 |
| TGWVKN | | | | | | | |
| PLNGAVHLNAQAQ | 98 | 1.725 | 0.037 | 0.739 | 4.830 | 21.040 | 28.572 |
| TGWVKN | | | | | | | |
| PLNGAVHLSAQAQ | 99 | 0.653 | 0.014 | 0.523 | 3.415 | 16.268 | 22.092 |
| TGWVKN | | | | | | | |
| PLNGAVHLYAEAQ | 100 | 0.752 | 0.016 | 0.000 | 0.000 | 0.825 | 1.120 |
| TGWVKN | | | | | | | |
| PLNGAVHLYAHAQ | 101 | 8.658 | 0.187 | 0.345 | 2.253 | 1.734 | 2.355 |
| TGWVKN | | | | | | | |
| PLNGAVHLYAKAQ | 649 | 28.559 | 0.615 | 0.278 | 1.817 | 1.532 | 2.081 |
| TGWVKN | | | | | | | |
| PLNGAVHLYALAQ | 102 | 2.311 | 0.050 | 0.000 | 0.000 | 9.892 | 13.433 |
| TGWVKN | | | | | | | |
| PLNGAVHLYAPAQ | 103 | 3.766 | 0.081 | 0.000 | 0.000 | 5.697 | 7.737 |
| TGWVKN | | | | | | | |
| PLNGAVHLYAQAE | 104 | 0.360 | 0.008 | 0.213 | 1.394 | 2.738 | 3.718 |
| TGWVKN | | | | | | | |
| PLNGAVHLYAQAQ | 105 | 7.865 | 0.169 | 0.099 | 0.648 | 1.486 | 2.018 |
| KGWVKN | | | | | | | |
| PLNGAVHLYAQAQ | 566 | 36.293 | 0.782 | 0.000 | 0.000 | 134.753 | 182.997 |
| LSPVKN | | | | | | | |
| PLNGAVHLYAQAQ | 960 | 15.955 | 0.344 | 0.000 | 0.000 | 51.408 | 69.813 |
| LSSVKN | | | | | | | |
| PLNGAVHLYAQAQ | 106 | 10.171 | 0.219 | 0.000 | 0.000 | 13.928 | 18.914 |
| PGWVKN | | | | | | | |
| PLNGAVHLYAQAQ | 107 | 3.450 | 0.074 | 0.000 | 0.000 | 7.922 | 10.758 |
| SGWVKN | | | | | | | |
| PLNGAVHLYAQAQ | 108 | 11.748 | 0.253 | 0.000 | 0.000 | 8.691 | 11.802 |
| SSLVKN | | | | | | | |
| PLNGAVHLYAQAQ | 109 | 5.856 | 0.126 | 0.169 | 1.107 | 10.452 | 14.194 |
| SSSVKN | | | | | | | |
| PLNGAVHLYAQAQ | 110 | 4.473 | 0.096 | 0.000 | 0.000 | 14.173 | 19.247 |
| TACVKN | | | | | | | |
| PLNGAVHLYAQAQ | 763 | 23.248 | 0.501 | 0.000 | 0.000 | 11.433 | 15.526 |
| TASVKN | | | | | | | |
| PLNGAVHLYAQAQ | 828 | 20.599 | 0.444 | 0.333 | 2.174 | 6.667 | 9.054 |
| TAWVKN | | | | | | | |
| PLNGAVHLYAQAQ | 111 | 0.721 | 0.016 | 0.000 | 0.000 | 14.566 | 19.781 |
| TEWVKN | | | | | | | |
| PLNGAVHLYAQAQ | 112 | 8.239 | 0.178 | 0.000 | 0.000 | 10.794 | 14.659 |
| TGAVKN | | | | | | | |
| PLNGAVHLYAQAQ | 113 | 8.144 | 0.175 | 0.222 | 1.452 | 14.865 | 20.187 |
| TGCVKN | | | | | | | |
| PLNGAVHLYAQAQ | 529 | 40.644 | 0.876 | 0.167 | 1.092 | 8.407 | 11.417 |
| TGGVKN | | | | | | | |
| PLNGAVHLYAQAQ | 114 | 10.090 | 0.217 | 0.145 | 0.945 | 10.946 | 14.864 |
| TGLVKN | | | | | | | |
| PLNGAVHLYAQAQ | 1100 | 13.059 | 0.281 | 0.000 | 0.000 | 2.607 | 3.541 |
| TGRVKN | | | | | | | |
| PLNGAVHLYAQAQ | 631 | 29.707 | 0.640 | 0.516 | 3.369 | 7.169 | 9.735 |
| TGSVKN | | | | | | | |
| PLNGAVHLYAQAQ | 765 | 23.167 | 0.499 | 0.260 | 1.700 | 10.937 | 14.852 |
| TGWVKN | | | | | | | |
| PLNGAVHLYAQAQ | 720 | 25.032 | 0.539 | 1.771 | 11.569 | 10.981 | 14.913 |
| TLSVKN | | | | | | | |
| PLNGAVHLYAQAQ | 1033 | 14.369 | 0.310 | 1.308 | 8.546 | 1.210 | 1.643 |
| TRWVKN | | | | | | | |
| PLNGAVHLYAQAQ | 700 | 26.099 | 0.562 | 0.000 | 0.000 | 29.844 | 40.528 |
| TSSVKN | | | | | | | |
| PLNGAVHLYAQAQ | 1037 | 14.347 | 0.309 | 0.000 | 0.000 | 12.484 | 16.953 |
| TSTVKN | | | | | | | |
| PLNGAVHLYAQAQ | 115 | 4.212 | 0.091 | 0.000 | 0.000 | 34.651 | 47.056 |
| TSWVKN | | | | | | | |
| PLNGAVHLYAQAQ | 625 | 30.131 | 0.649 | 0.000 | 0.000 | 22.666 | 30.781 |
| TVAVKN | | | | | | | |
| PLNGAVHLYAQAQ | 325 | 69.266 | 1.492 | 0.231 | 1.508 | 22.335 | 30.332 |
| TVSVKN | | | | | | | |
| PLNGAVHLYAQAQ | 771 | 22.784 | 0.491 | 0.000 | 0.000 | 1.612 | 2.189 |
| TVWVKN | | | | | | | |
| PLNGAVHLYAQAQ | 610 | 31.581 | 0.680 | 0.354 | 2.310 | 21.595 | 29.326 |
| VSAVKN | | | | | | | |
| PLNGAVHLYAQAQ | 558 | 37.158 | 0.801 | 0.000 | 0.000 | 40.525 | 55.033 |
| VSPVKN | | | | | | | |
| PLNGAVHLYAQEQ | 116 | 7.694 | 0.166 | 0.000 | 0.000 | 1.548 | 2.102 |
| TGWVKN | | | | | | | |
| PLNGAVHLYAQSQ | 117 | 7.014 | 0.151 | 0.000 | 0.000 | 5.931 | 8.054 |
| TGWVKN | | | | | | | |
| PLNGAVHLYDQAQ | 118 | 2.761 | 0.059 | 0.146 | 0.956 | 7.745 | 10.518 |
| TGWVKN | | | | | | | |
| PLNGAVHLYEQAQ | 119 | 7.658 | 0.165 | 0.000 | 0.000 | 7.124 | 9.675 |
| TGWVKN | | | | | | | |
| PLNGAVHLYGQAQ | 120 | 0.874 | 0.019 | 0.000 | 0.000 | 5.745 | 7.802 |
| TGWVKN | | | | | | | |
| PLNGAVHLYSQAQ | 121 | 6.910 | 0.149 | 0.000 | 0.000 | 11.682 | 15.865 |
| TGLVKN | | | | | | | |
| PLNGAVHLYSQAQ | 122 | 7.536 | 0.162 | 0.245 | 1.599 | 4.415 | 5.996 |
| TGWVKN | | | | | | | |
| PLNGAVHLYTQAQ | 123 | 0.595 | 0.013 | 0.166 | 1.086 | 10.157 | 13.793 |
| TGWVKN | | | | | | | |
| PLNGAVHPYAQAQ | 124 | 0.788 | 0.017 | 0.178 | 1.162 | 3.527 | 4.790 |
| TGWVKN | | | | | | | |
| PLNGAVHRSLQAQ | 125 | 0.153 | 0.003 | 0.449 | 2.931 | 7.938 | 10.780 |
| TGWVKN | | | | | | | |
| PLNGAVHRYAQAQ | 126 | 1.878 | 0.040 | 0.000 | 0.000 | 2.446 | 3.322 |
| TGWVKN | | | | | | | |
| PLNGAVHVYAQAQ | 127 | 1.937 | 0.042 | 0.123 | 0.802 | 7.871 | 10.689 |
| TGWVKN | | | | | | | |
| PLNGAVLLYAQAQ | 128 | 4.149 | 0.089 | 0.211 | 1.379 | 3.878 | 5.266 |
| TGWVKN | | | | | | | |
| PLNGAVNLYAQAQ | 129 | 3.505 | 0.076 | 0.000 | 0.000 | 1.145 | 1.555 |
| TGCVKN | | | | | | | |
| PLNGAVNLYAQAQ | 130 | 4.586 | 0.099 | 0.262 | 1.710 | 2.124 | 2.884 |
| TGWVKN | | | | | | | |
| PLNGAVNLYDQAQ | 131 | 2.486 | 0.054 | 0.000 | 0.000 | 9.468 | 12.858 |
| TGWVKN | | | | | | | |
| PLNGAVPLYAQAQ | 132 | 1.622 | 0.035 | 0.000 | 0.000 | 7.002 | 9.509 |
| TGWVKN | | | | | | | |
| PLNGAVQLYAQAQ | 133 | 2.014 | 0.043 | 0.381 | 2.487 | 11.888 | 16.145 |
| TGWVKN | | | | | | | |
| PLNGAVRSTAQAQ | 134 | 0.505 | 0.011 | 0.662 | 4.326 | 24.950 | 33.883 |
| TGWVKN | | | | | | | |
| PLNGAVSLRAQAQ | 135 | 0.725 | 0.016 | 1.267 | 8.277 | 6.291 | 8.543 |
| TGWVKN | | | | | | | |
| PLNGAVSSRAQAQ | 136 | 0.739 | 0.016 | 1.091 | 7.130 | 19.478 | 26.452 |
| TGWVKN | | | | | | | |
| PLNGDVHLYAQAQ | 14 | 2.905 | 0.063 | 0.000 | 0.000 | 10.919 | 14.829 |
| TGCVKN | | | | | | | |
| PLNGDVHLYAQAQ | 15 | 2.559 | 0.055 | 0.235 | 1.539 | 9.737 | 13.224 |
| TGWVKN | | | | | | | |
| PLNGGVHLYAQAQ | 864 | 19.077 | 0.411 | 0.292 | 1.905 | 4.576 | 6.214 |
| TGWVKN | | | | | | | |
| PLNGPVHLYAQAQ | 16 | 2.216 | 0.048 | 0.000 | 0.000 | 5.169 | 7.019 |
| TGWVKN | | | | | | | |
| PLNGSVHLYAQAQ | 17 | 5.707 | 0.123 | 0.000 | 0.000 | 21.281 | 28.900 |
| TGCVKN | | | | | | | |
| PLNGSVHLYAQAQ | 791 | 22.050 | 0.475 | 0.184 | 1.202 | 13.110 | 17.803 |
| TGWVKN | | | | | | | |
| PLNGTVHLYAQAQ | 978 | 15.482 | 0.334 | 0.000 | 0.000 | 17.576 | 23.868 |
| TGWVKN | | | | | | | |
| PLNSAVHLYAQAQ | 1174 | 1.468 | 0.032 | 0.000 | 0.000 | 3.674 | 4.989 |
| TGWVKN | | | | | | | |
| PLSGAVHLYAQAQ | 1175 | 0.680 | 0.015 | 0.000 | 0.000 | 8.111 | 11.015 |
| TGWVKN | | | | | | | |
| PPNGAVHLYAQAQ | 1176 | 0.829 | 0.018 | 0.546 | 3.567 | 9.008 | 12.233 |
| TGWVKN | | | | | | | |
| PRNGAVHLYAQAQ | 1177 | 1.194 | 0.026 | 0.421 | 2.751 | 9.143 | 12.417 |
| TGWVKN | | | | | | | |
| PVNGAVHLYAQAQ | 1178 | 8.167 | 0.176 | 0.145 | 0.946 | 11.979 | 16.268 |
| TGWVKN | | | | | | | |
| QLNGAVHIYAQAQ | 1179 | 6.982 | 0.150 | 0.000 | 0.000 | 14.405 | 19.563 |
| TGWVKN | | | | | | | |
| QLNGAVHLYAQAQ | 1180 | 2.446 | 0.053 | 0.000 | 0.000 | 14.453 | 19.627 |
| TGCVKN | | | | | | | |
| QLNGAVHLYAQAQ | 1181 | 7.518 | 0.162 | 0.000 | 0.000 | 1.613 | 2.190 |
| TGLVKN | | | | | | | |
| QLNGAVHLYAQAQ | 966 | 15.815 | 0.341 | 1.130 | 7.380 | 3.272 | 4.443 |
| TGSVKN | | | | | | | |
| QLNGAVHLYAQAQ | 1032 | 14.378 | 0.310 | 0.137 | 0.893 | 10.495 | 14.253 |
| TGWVKN | | | | | | | |
| QLNGAVHLYDQAQ | 1182 | 2.378 | 0.051 | 0.000 | 0.000 | 1.224 | 1.663 |
| TGWVKN | | | | | | | |
| RLNGAVHLYAQAQ | 1183 | 3.131 | 0.067 | 0.000 | 0.000 | 4.894 | 6.646 |
| TGWVKN | | | | | | | |
| SINGAVHLYAQAQ | 1184 | 1.234 | 0.027 | 0.409 | 2.671 | 4.660 | 6.328 |
| TGWVKN | | | | | | | |
| SLNGAVHLYAQAQ | 1185 | 6.104 | 0.132 | 0.328 | 2.143 | 11.122 | 15.104 |
| TGWVKN | | | | | | | |
| SLNGAVNLYAQAQ | 1186 | 1.387 | 0.030 | 0.833 | 5.444 | 5.215 | 7.083 |
| TGWVKN | | | | | | | |
| TLNGAVHLYAQAQ | 1187 | 2.176 | 0.047 | 0.108 | 0.707 | 5.355 | 7.272 |
| TGWVKN | | | | | | | |
| TLNGAVNLYAQAQ | 1188 | 1.333 | 0.029 | 0.000 | 0.000 | 1.292 | 1.754 |
| TGWVKN | | | | | | | |
| PLNGAVHHYAQAQ | 1189 | 0.937 | 0.020 | 0.000 | 0.000 | 982.466 | 1334.208 |
| TGWVPN | | | | | | | |
| PLNGAVHLNAQAQ | 1190 | 3.698 | 0.080 | 3.072 | 20.072 | 5448.821 | 7399.606 |
| TGWVPN | | | | | | | |
| PLNGAVHLSAQAQ | 1191 | 3.356 | 0.072 | 3.362 | 21.966 | 5527.158 | 7505.989 |
| TGWVPN | | | | | | | |
| PLNGAVHLYAQAQ | 1192 | 8.959 | 0.193 | 0.969 | 6.329 | 3019.591 | 4100.664 |
| TGCVPN | | | | | | | |
| PLNGAVHLYAQAQ | 314 | 71.770 | 1.546 | 2.071 | 13.534 | 2408.300 | 3270.518 |
| TGWVPN | | | | | | | |
| PLNGAVNLYAQAQ | 1193 | 5.356 | 0.115 | 1.809 | 11.822 | 2103.432 | 2856.501 |
| TGWVPN | | | | | | | |
| PLNGTVHLYAQAQ | 885 | 18.270 | 0.394 | 0.000 | 0.000 | 4688.758 | 6367.425 |
| TGWVPN | | | | | | | |
| PVNGAVHLYAQAQ | 1016 | 14.568 | 0.314 | 0.000 | 0.000 | 1269.496 | 1724.000 |
| TGWVPN | | | | | | | |
| QLNGAVHLYAQAQ | 648 | 28.572 | 0.616 | 0.000 | 0.000 | 14152.524 | 19219.405 |
| TGWVPN | | | | | | | |
| SLNGAVHLYAQAQ | 1058 | 13.914 | 0.300 | 1.876 | 12.256 | 1800.081 | 2444.545 |
| TGWVPN | | | | | | | |
| PLNGAVHLYAQAQ | 476 | 46.410 | 1 | 0.153 | 1 | 0.736 | 1 |
| TGWVQN | | | | | | | |
| AQAQAQTGWVQN | 1194 | 1 | 0.0215 | 1 | 6.533 | 1 | 1.358 |

### Example 8. Maturation of TTD-001 Capsid in Mice

This Example describes maturation of the TTD-001 (SEQ ID NO: 3623 (DNA) and 3636 (amino acid), comprising SEQ ID NO: 1725 or 3648) capsid variant to further enhance its transduction and biodistribution in the central nervous system and evolve the AAV capsid variants in mice. Two approaches were used to mature the TTD-001 capsid sequence in order to randomize and mutate within and around the peptide insert comprised within loop VIII of the capsid variant. In the first maturation approach, sets of three contiguous amino acids were randomized across the mutagenesis region in the TTD-001 sequence, which spanned from position 587 to position 602, numbered according to SEQ ID NO: 3636. In the second maturation approach, mutagenic primers were used to introduce point mutations at a low frequency, scattered across the mutagenesis region in the TTD-001 sequences ranging from position 587 to position 602, numbered according to SEQ ID NO: 3636. AAV capsid variants arising from each maturation approach for TTD-001 were pooled together, for subsequent testing and characterization in mice.

The library of pooled matured AAV capsid variants generated from TTD-001 matured AAV capsid variant were injected into 3 mice. After a period in life, the brains of the mice were isolated and RNA was extracted. Following RNA recovery and RT-PCR amplification, a systematic NGS enrichment analysis was performed to calculate the fold enrichment ratio relative to an AAV9 control, and the peptides comprised within the variants were identified. The coefficient of variance (CV) was calculated for each peptide across the samples isolated from the mice, and those that had a CV value <1 were identified, as these were the peptides that were reliably detected in most or all of the brain samples isolated from the mice. These TTD-001 matured capsid variants and their peptide sequences are provided in Table 11.

Over 3000 TTD-001 matured capsid variants demonstrated increased expression relative to the wild-type AAV9 control. As shown in Table 11, approximately 34 demonstrated greater than a ten-fold enrichment relative to the wild-type AAV9 control. Also, across the peptides comprised within the TTD-001 matured capsid variants with the greatest fold-enrichment relative to the wild-type AAV9 control capsid in the mouse brain, it was observed that the modifications in the variant sequences appeared in the middle of the sequence, specifically at residues corresponding to positions 591-593, relative to a reference sequence numbered according to the amino acid sequence of SEQ ID NO: 3636.

Taken together, these data demonstrate that following two maturation approaches, matured TTD-001 capsid variants with loop VIII modifications were generated with significantly enhanced CNS tropism in mice compared to a wild-type AAV9 control capsid.

TABLE 11
NGS fold-enrichment of TTD-001 matured AAV
capsid variants in the brains of mice

| | SEQ | | Fold |
|---|---|---|---|
| | ID | | enrichment |
| Peptide Sequence | NO: | CV | over AAV9 |
| PLNNPGHLYAQAQTGW | 1139 | 0.696 | 94.10 |
| PLNGVKALYAQAQTGW | 1140 | 0.047 | 78.63 |
| PLNGARMIYAQAQTGW | 1141 | 0.530 | 66.48 |
| PLNGVKMLYAQAQTGW | 1142 | 0.203 | 60.88 |
| PLNGVRALYAQAQTGW | 1143 | 0.674 | 60.72 |
| PLNGVRSLYAQAQTGW | 1144 | 0.400 | 52.99 |
| PLNGTRMLYAQAQTGW | 1145 | 0.517 | 50.75 |
| PLNGVRTLYAQAQTGW | 1146 | 0.698 | 42.02 |
| PLNGVRMLYAQAQTGW | 1147 | 0.678 | 31.62 |
| PLNGARMLYAQAQTGW | 1148 | 0.186 | 30.79 |
| PLNGAKMLYAQAQTGW | 1149 | 0.318 | 28.20 |
| PLNGVKSLYAQAQTGW | 1150 | 0.284 | 27.59 |
| PLNGVQMLYAQAQTGW | 1151 | 0.316 | 27.39 |
| PSARAVHLYAQAQTGW | 1152 | 0.893 | 24.75 |
| PLNGTRSLYAQAQTGW | 1153 | 0.459 | 21.66 |
| PLNGVRQLYAQAQTGW | 1154 | 0.190 | 20.62 |
| PLNGAQMLYAQAQTGW | 1155 | 0.295 | 19.61 |
| PLNGVKYLYAQAQTGW | 1156 | 0.842 | 19.25 |
| PLNGARTIYAQAQTGW | 1157 | 0.371 | 17.48 |
| TLNGAVHLNAQGQTGW | 1158 | 0.891 | 16.19 |
| PLNGAGADYAQAQTGW | 1159 | 0.768 | 15.41 |
| PLNMPGHLYAQAQTGW | 1160 | 0.729 | 14.98 |
| PLNGVQALYAQAQTGW | 1161 | 0.359 | 13.56 |
| SLNGAVHLYVQAQTGS | 1162 | 0.866 | 12.73 |
| PLNGVKTLYAQAQTGW | 1163 | 0.239 | 12.54 |
| SLNGAVHLSAQAQAGW | 1164 | 0.920 | 12.45 |
| SINGPVHLYAQAQTGW | 1165 | 0.409 | 12.14 |
| PLNGGVHLSVQAQTGW | 1166 | 0.979 | 12.10 |
| PLNGAGHLNAQAQTRW | 1167 | 0.983 | 11.71 |
| ALNGAVHVSAQAQTGW | 1168 | 0.645 | 10.75 |
| PLNLVGHLYAQAQTGW | 1169 | 0.872 | 10.63 |
| PLNLTGHLYAQAQTGW | 1170 | 0.341 | 10.38 |
| PGRQAVHLYAQAQTGW | 1171 | 0.971 | 10.13 |
| LVNSTVRLNAQVQTGW | 1172 | 0.878 | 10.04 |
| AQAQAQTGW | 1173 | 0.299 | 1.0 |

### Example 9. Optimization of rAAV Particle Formulations

The aim of this experiment was to obtain a formulation that could support a viral concentration >1×10¹³ vg/mL, minimize aggregation (e.g., to <5%, as assessed by SEC-FLD), and maintain osmolality within a certain range (e.g., about 250-600 mOsm/kg for isotonic solutions) under various storage conditions. An AAV9 variant (TTD-001) with an SEAP/GFP payload was used in these experiments.

Various parameters of a modified PBS-based formulation (PBS and 0.001% Pluronic F68, pH7.4) for therapeutic recombinant AAV particles comprising an AAV9 capsid or AAV9 capsid variant (or variants thereof), e.g., an AAV capsid variant described herein, including excipients, crowding agents, pH, and osmolality, were altered and tested for their impact as discussed below.

Stability of the new formulations was tested under various conditions, including storage at −80° C., 2-8° C., room temperature (25° C.), as well as under multiple freeze thaw cycles. Other characteristics of the formulations were also examined as follows: pH (pH meter), osmolality (osmometer), VP1,2,3% purity (capillary gel electrophoresis-sodium dodecyl sulfate (CE-SDS)), pI/surface charge (capillary isoelectric focusing (CIEF)), vector titer (qPCR), sub-μm aggregation (size exclusion chromatography-fluorescence detection (SEC-FLD)/dynamic light scattering (DLS)), and % occupancy (SEC-MALS).

Components of the tested formulations are shown in Table 12.

TABLE 12
Formulation components

| | |
|---|---|
| Modified PBS | PBS, pH 7.4 |
| | 0.001% Pluronic F-68 |
| 1% glycerol | 20 mM Tris, pH 8.0 |
| | 62.5 mM NaCl |
| | 1% Glycerol |
| | 0.001% Pluronic F-68 |
| 2.5% glycerol | 20 mM Tris, pH 8.0 |
| | 62.5 mM NaCl |
| | 2.5% Glycerol |
| | 0.001% Pluronic F-68 |
| 5.95% trehalose | 20 mM Tris, pH 8.0 |
| | 62.5 mM NaCl |
| | 5.95% Trehalose |
| | 0.001% Pluronic F-68 |

The analysis of VP1,2,3 ratio and % purity of TTD-001 in buffer exchanged samples was performed as follows. Briefly, 100 μl of sample was combined with 350 μl SDS sample buffer and added to a pretreated Amicon Ultra 0.5 ml Centrifugal Spin Filter (10 kDa). Samples were spun, filters were inverted into a new collection tube and spun, and the volume was brought up to 95 μl with SDS sample buffer. 2 μl of 10 kDa internal standard and 5 μL of iodoacetamide (IAM) were added to each sample and they were alkylated by heating to 70° C. for 3 minutes before cooling to room temperature. Samples were then transferred to CE vial with a PCR tube inside for analysis by CE-SDS.

With the exception of the sample in modified PBS (for which the signal was significantly lower than the other samples, likely due to loss of material following aggregation), all buffer exchanged samples retained the VP1,2,3 ratios of the original starting material. Properties of the buffer exchanged formulations at time 0 and at 3 months (for samples stored at −80° C. and 2-8° C.) are shown in Table 13 (VP ratio and % purity). Storage at −80° C. over 3 months had no impact based on VP ratio or % purity. With storage at 2-8° C., a significant impact on the capsid structure emerged over 2 weeks, seen as additional peaks (possible clipped VPs) in CESDS electropherograms and decrease in overall % purity (Table 13). The average VP ratios also shifted, seen with a drop in VP1 levels, likely due to increased loss of capsids with higher VP1 content. Under each condition, the signal for the modified PBS formulation in CESDS electropherograms remained an order of magnitude lower (reflected by differences in titer), suggesting degradation was prevalent.

TABLE 13
Capsid parameters

| | t = 0 | | t = 3 months (−80° C.) | | t = 3 months (2-8° C.) | |
|---|---|---|---|---|---|---|
| Sample | VP Ratio* | % Purity | VP Ratio | % Purity | VP Ratio | % Purity |
| Modified PBS | 32:2:1 | 100 | 47:2:1 | 93.6 | 54:2:1 | 100 |
| 5.95% Trehalose | 32:2:1 | 100 | 34:2:1 | 97.3 | 82:3:1 | 61.3 |
| 2.5% Glycerol | 31:2:1 | 100 | 32:2:1 | 100 | 60:3:1 | 81.6 |
| 1% Glycerol | 30:2:1 | 100 | 28:2:1 | 100 | 57:3:1 | 87.7 |

*VP ratios presented as VP3:VP2:VP1

Table 14 shows pI parameters (Central pI value, pI range, and area %) of samples that have been buffer exchanged, as assessed by cIEF. Briefly, 10 μl of each sample was mixed with 200 μl of cIEF master mix. In tandem, 200 μl of cIEF master mix was added to 1.0 μl of pI markers 5.5, 7.0, and 9.5. Formulation samples and marker standards were centrifuged, and 200 μl from each were added to a CE vial with a PCR tube inside for analysis by cIEF.

With the exception of the modified PBS formulation, which caused significant loss of signal (as reflected in qPCR measurements) along with a shift to higher pI values, the three formulations (5.95% trehalose, 2.5% glycerol, and 1% glycerol) retained the same charge profile as the original material, with a strong signal at the expected pI range.

TABLE 14
pI parameters

| | t = 0 | | | t = 3 months (−80° C.) | | | t = 3 months (2-8° C.) | | |
|---|---|---|---|---|---|---|---|---|---|
| | Central | pI | Area | Central | pI | Area | Central | pI | Area |
| Sample | pI value | range | % | pI value | range | % | pI value | range | % |
| Modified PBS | 7.4 | 7.1-7.8 | 100.0 | 6.5 | 6.2-7.5 | 100.0 | 6.4 | 6.2-6.6 | 100.0 |
| 5.95% Trehalose | 6.5 | 6.2-6.7 | 100.0 | 6.2 | 6.0-6.5 | 100.0 | 6.0 | 5.9-6.2 | 100.0 |
| 2.5% Glycerol | 6.5 | 6.2-6.7 | 100.0 | 6.2 | 6.0-6.5 | 100.0 | 6.0 | 5.9-6.2 | 100.0 |
| 1% Glycerol | 6.4 | 6.2-6.7 | 100.0 | 6.2 | 5.8-6.6 | 100.0 | 6.0 | 5.9-6.2 | 100.0 |

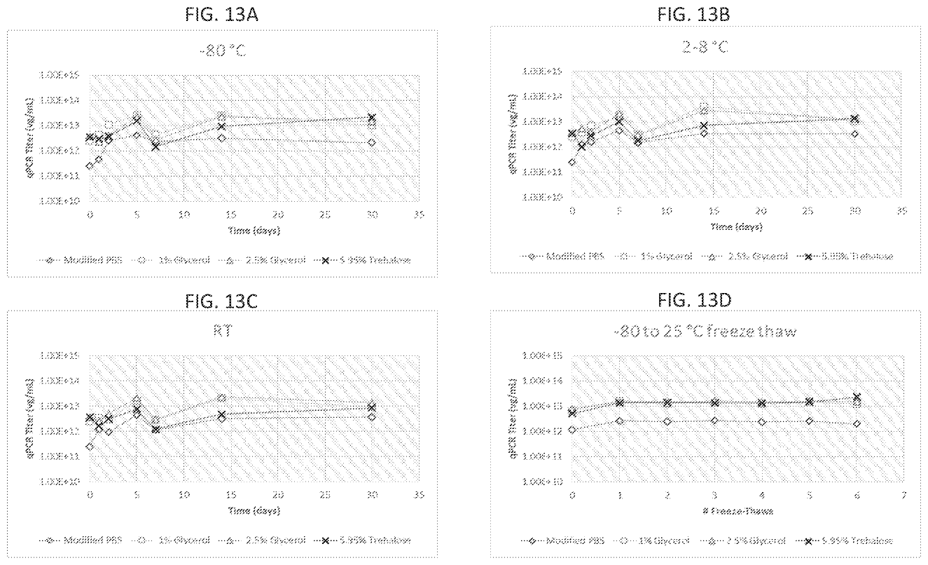

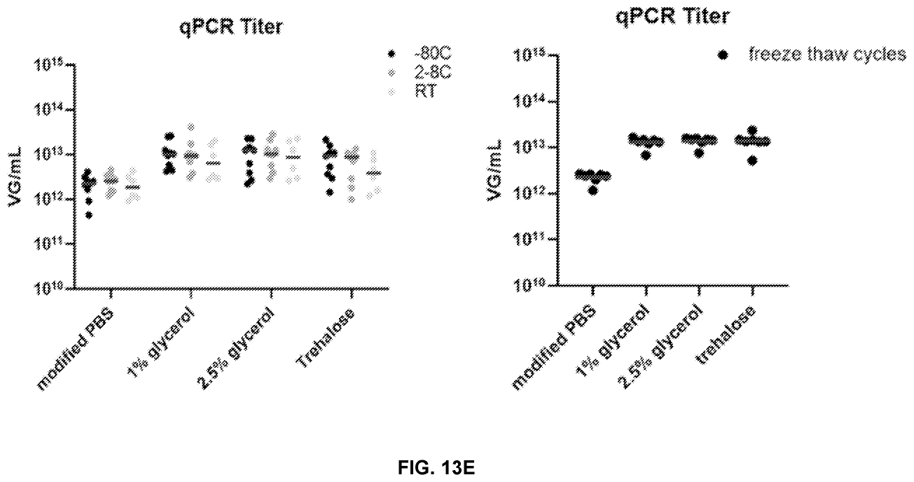

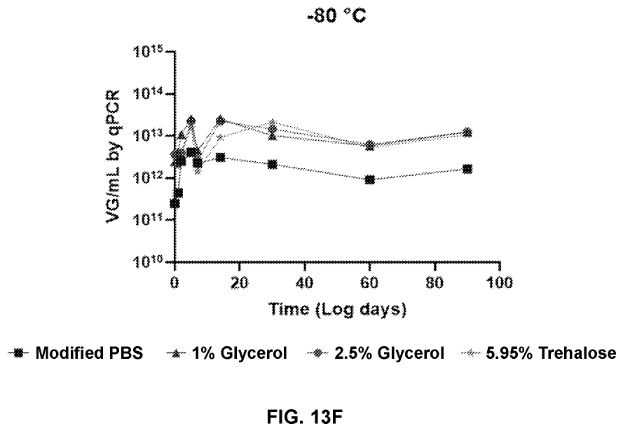

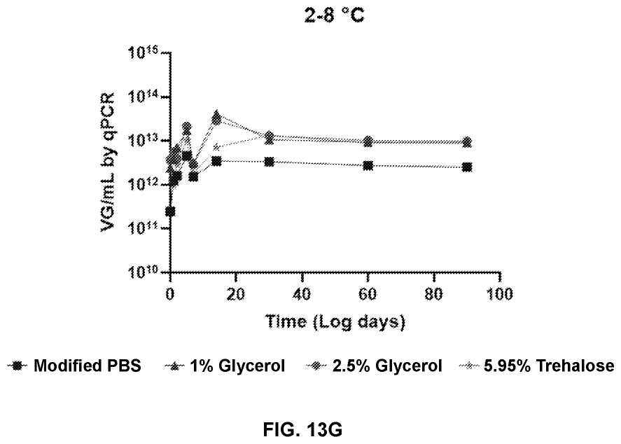

Tables 15-19 and FIGS. 13A-13D, 14A-14D, and 15A-15D show the various properties of the tested formulations and TTD-001 particles, including pH, osmolality, titer, aggregation, and occupancy. pH was assessed using a pH meter, and osmolality with an osmometer.

Viral titer for each sample was determined through qPCR. Briefly 5 μl of each sample was combined with 95 μl of DNase reaction mixture (0.3 mg/mL DNase I in QPCR DNase buffer) in a 96-well PCR plate and incubated at 37° C. for 1 hour to remove any external DNA. 125 μl of proteinase K mixture (0.96 mg/mL proteinase K and 20 mM EDTA in QPCR proteinase K buffer) was added to each sample. The plate was incubated at 55° C. for 1 hour and 95° C. for 10 minutes, to deactivate the DNase I and denature the capsid, releasing the DNA. 4 μl of each sample and standard were combined with 16 μl qPCR master mix (1.25× Taqman fast advanced master mix, 1.25× primer probe mixture in nuclease free water). The plate was placed inside a light cycler 480 instrument and the following cycling program was used: Denature (95° C. 10 minutes), Cycle 45 times (95° C. 10 seconds, 60° C. 10 seconds, 72° C. 10 seconds). This was followed by acquisition of the fluorescent signals on the final step. The data were processed using the light cycler 480 software to generate titer values in vg/ml.

Genomic integrity in terms of the number of bands and relative intensity was measured using an agarose gel run under alkaline denaturing conditions with SYBR gold fluorescent staining. Briefly a 1% agarose gel was cast in 1×Tris acetate EDTA (TAE) solution. 10 μl of each sample was combined with 10 μl 2× denaturing loading dye and the samples were heated to 95° C. for 10 minutes and cooled to room temperature. From this, 10 μl of each sample mixture was loaded into separate wells along with 5 μl generuler as a size ladder. The gel was run at 40V for 16 hours at 2-8° C. Following a wash with 1×TAE solution, it was stained using SYBR gold solution and imaged using a blue filter (460 nm). The size and band intensities were determined using image J software.

As shown in the Tables and FIGS. 13A-13G, the addition of glycerol and trehalose improved viral titers and genomic integrity across various stability tests compared to the modified PBS formulation, including samples stored at −80° C., 2-8° C., and room temperature, and under multiple rounds of freeze thaw. When titer values were plotted as a function of time and separated into the individual temperature arms, the glycerol-containing formulations and trehalose-containing formulation were able to maintain titers of 1E+13 VG/ml across each condition, i.e., −80° C., 2-8° C., 25° C., and under 6 freeze thaw cycles (FIG. 13E). Moreover, these titers were maintained at day 90 for the −80° C. and 2-8° C. conditions (FIGS. 13F and 13G). In DNA denaturing gel analysis, while all samples showed two DNA species (4.0 & 3.0 kbp), the main DNA species intensity was greater in the glycerol-containing formulations (1% and 2.5%) and 5.95% trehalose-containing formulation compared to the modified PBS formulation. No significant loss in intensity or emergence of additional species was observed for the glycerol-containing formulations and trehalose-containing formulation for time points spanning 2 weeks and 6 freeze thaw cycles.

Aggregation (% HMW) and occupancy (% full AAV capsids) were assessed using SEC-FLD and SEC-MALS. This assay utilizes size exclusion chromatography to separate AAV monomers from HMW aggregates, thereby quantifying the latter using the integrals of the fluorescent signal. Online multi-angle light scattering combined with refractive index measurements were used to determine the molar mass, geometry, and protein fraction of the aggregates. % Full was measured by using differences in the extinction coefficients at 280 nm to deconvolute the molar mass of the monomer into contributions from the protein (capsid) and payload (DNA). Briefly, sample were added to HPLC vials and loaded in an HPLC autosampler at 4° C. Samples were run on a 7.8×300 mm, 500 Å column with the following HPLC method parameters: instrument method (SEC 260 and 280), Pump (1.0 ml/min SEC mobile phase (2×DPBS, pH7.5)), Injection (50 μl of H₂O, formulation buffer, reference standard or sample), run time (20 min), DAD UV (280 nM), VWD UV (260 nM), FLD (Ex=280 Em=350, PMT gain=8), column temp (25° C.). Data (fluorescent signal) was analyzed using Chemstation. The signal was integrated manually from 4.5 to 10 minutes, and the integrated area was split using the split peak function to separate monomer (typically RT=7.0 min) and HMW peaks (typically RT=6.1 min). The integration and peak splitting was repeated for both UV signals (260 and 280 nm). % HMW from the fluorescence trace and A260/280 values were averaged across duplicate injections.

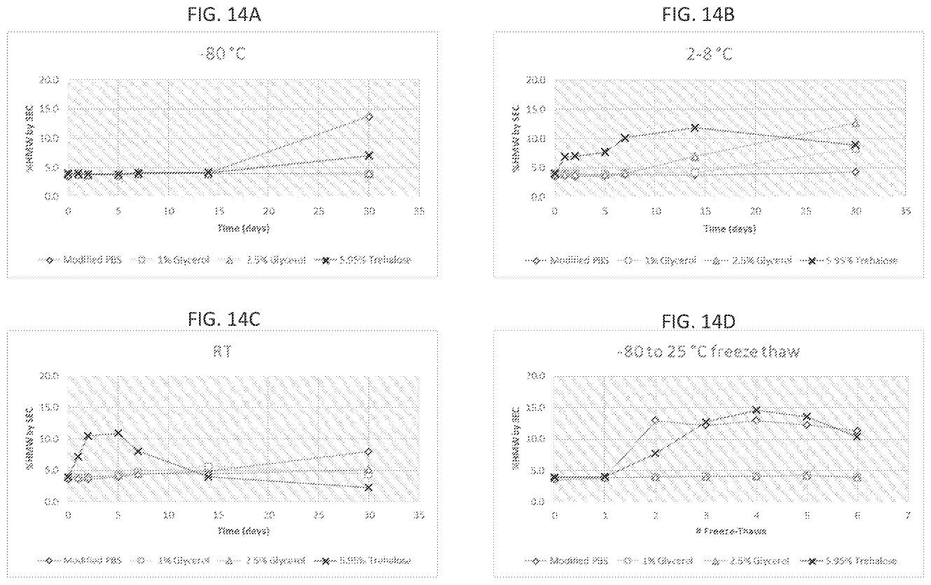

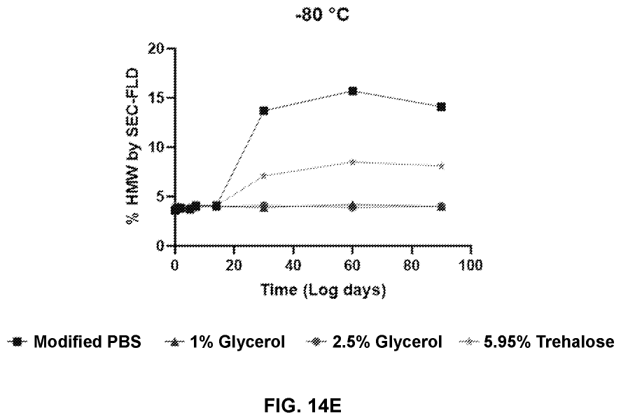

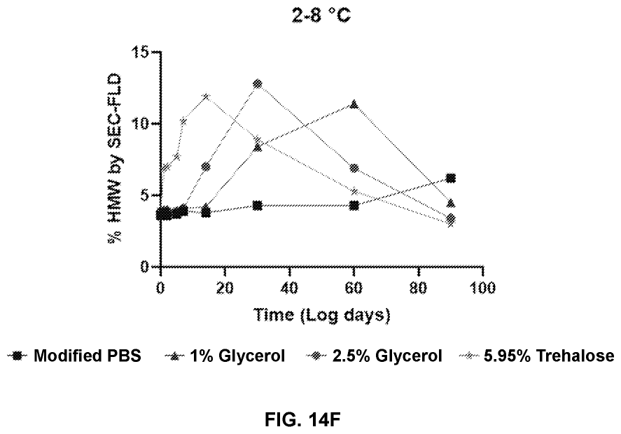

With regard to aggregation, glycerol-containing formulations (1% and 2.5%) showed less rAAV particle aggregation than the modified PBS formulation and 5.95% trehalose-containing formulation under certain conditions, for example, in samples stored for 30 days and 50 days at −80° C. (FIGS. 14A and 14E) and after multiple freeze thaw cycles (FIG. 14D). At −80° C., glycerol-based formulations displayed no change in the levels of % HMW aggregates over a 3 month time course, in contrast to both trehalose- and mPBS-containing formulations, where sharp increases in % HMW started at about 2 weeks (FIGS. 14A and 14E). In samples stored at 2-8° C., an immediate increase in aggregation level was observed in the trehalose-containing formulation, and also 2 weeks later in the glycerol-containing formulations; less rAAV particle aggregation than the modified PBS formulation were observed for both the glycerol- and trehalose-containing formulations in samples stored for 90 days (FIGS. 14B and 14F). The 5.95% trehalose-containing formulation also showed higher aggregation early on relative to the glycerol-containing formulations (1% and 2.5%) in samples stored at room temperature (FIG. 14C). With regard to multiple freeze-thaw cycles, glycerol-containing formulations remaining stable, indicating good compatibility for storage at −80° C. Both the trehalose-containing formulation and modified PBS formulation showed substantial increases in % HMW after two freeze-thaw cycles (FIG. 14D).

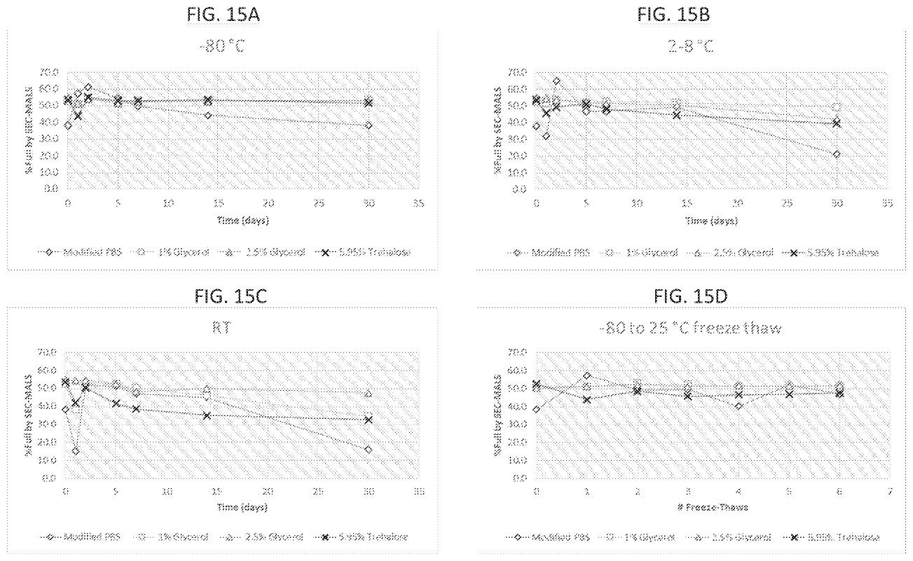

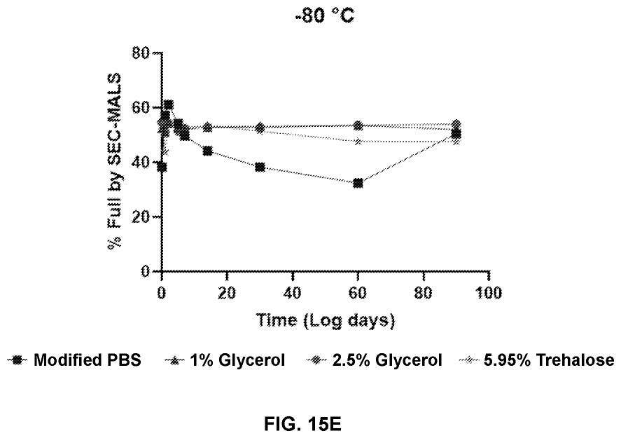

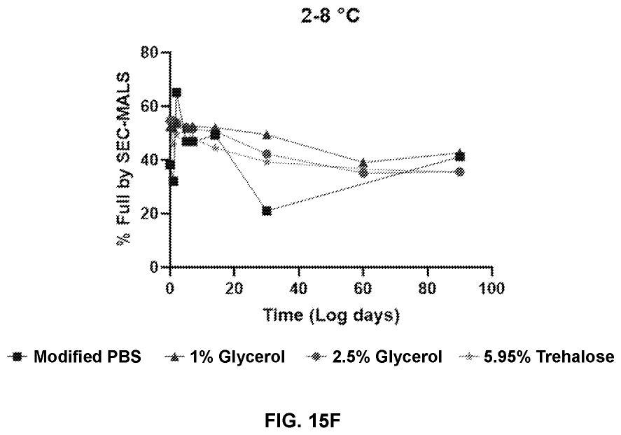

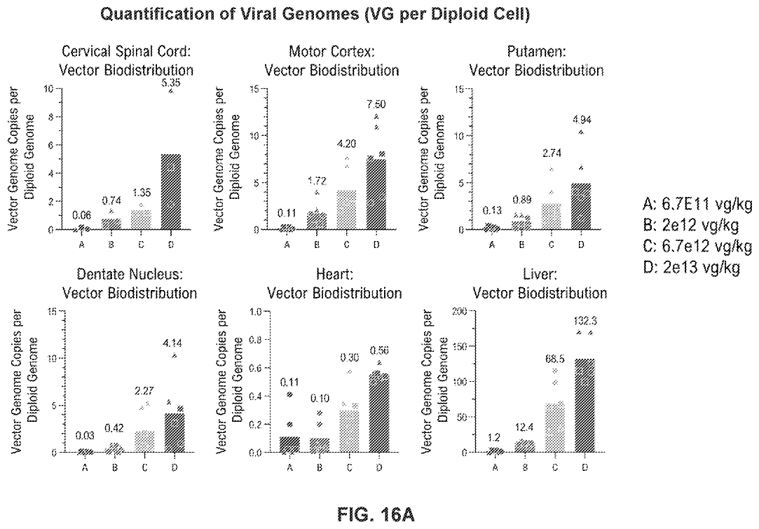

With regard to occupancy, as shown in the Tables and FIGS. 15A-15D, glycerol-containing formulations (1% and 2.5%) generally showed higher occupancy (% full AAV) relative to the modified PBS formulation, particularly at later time points under the tested storage conditions. While the 5.95% trehalose-containing formulation also showed similar occupancy to the glycerol formulations at −80° C., a loss was observed at the later time points at 2-8° C. and 25° C. Viral material in glycerol-based vehicle stored at −80° C., or challenged with 6 freeze-thaw cycles, maintained % full values over three months (FIGS. 15A and 15E). When stored at 2-8° C., the trehalose-containing formulation and modified PBS formulation showed an occupancy reduction (cargo loss) at 2 weeks and 1 month, respectively (FIGS. 15B and 15F). Storage at 2-8° C. had a smaller effect than storage at 25° C., producing a wider distinction between these groupings. At room temperature, the trehalose-containing formulation begins losing occupancy after 1 week, while the glycerol formulations maintain their integrity up to 2 weeks. A concentration of 2.5% glycerol vehicle retains both % full values of 50% at 25° C. up to 1 month (FIG. 15C).

For each formulation, all conditions showed no change in the solution state that could be observed by visible inspection. Measurement of the buffering properties (Tables 15-19) indicated no deviation from the target pH or osmolality, with average values of 408, 309, 536 and 378 mOsmo/kg for the mPBS, 1% glycerol, 2.5% glycerol and 5.95% trehalose vehicles respectively.

Overall, the glycerol-containing formulations were superior in that they remained stable for prolonged periods under the tested conditions while maintaining desirable properties (e.g., high viral titers, less aggregation, and higher % full capsids) relative to the modified PBS formulation. The 5.95% trehalose-containing formulation also remained stable for prolonged periods under many of the tested conditions.

TABLE 15
Stability of modified PBS formulation

| | | | | | Payload | | | | | | |
|---|---|---|---|---|---|---|---|---|---|---|---|
| | | | | | | Main | | | | | |
| | | | | | | DNA | | DLS | | | SEC |
| | | Time | Vehicle | | | Species | Occupancy | | | % | % |
| Formulation | Condition | (days) | pH | Osmo | qPCR | Intensity | % Full | rH | % pd | intensity | HMW |
| Modified PBS | −80° C. | t0 | 7.3 | 393 | 2.47E+11 | 3914 | 38.2 | 14.8 | 11.5 | 100 | 3.6 |
| | | 1 | 7.3 | 390 | 4.45E+11 | 4298 | 57.2 | 12.7 | 4 | 100 | 3.8 |
| | | 2 | 7.3 | 390 | 2.51E+12 | 4016 | 61.1 | 13.2 | 10 | 100 | 3.8 |
| | | 5 | 7.4 | 497 | 4.08E+12 | 4416 | 54.2 | 13.9 | 11.9 | 100 | 3.7 |
| | | 7 | 7.4 | 393 | 2.24E+12 | 3838 | 49.6 | 12.3 | 7 | 100 | 4.3 |
| | | 14 | 7.4 | 436 | 3.10E+12 | 3943 | 44.2 | 15.4 | 8 | 100 | 5.0 |
| | | 30 | 7.2 | 365 | 2.12E+12 | | 38.2 | 14.2 | 11.8 | 100 | 14.2 |
| | | 60 | 7.2 | 389 | 9.09E+11 | | 32.4 | 13.0 | 8.7 | 100 | 15.7 |
| | | 90 | 7.3 | 401 | 1.66E+12 | | 50.5 | 15.2 | 9.5 | 94.6 | 14.1 |
| | 2-8° C. | 1 | 7.0 | 401 | 1.23E+12 | 4468 | 32.1 | 14.7 | 11.2 | 100 | 3.8 |
| | | 2 | 7.3 | 394 | 1.59E+12 | 3291 | 65.1 | 15 | 10.2 | 100 | 3.6 |
| | | 5 | 7.4 | 395 | 4.57E+12 | 2909 | 46.8 | 14.7 | 11.2 | 100 | 3.7 |
| | | 7 | 7.3 | 392 | 1.52E+12 | 3767 | 46.8 | 14.8 | 10.7 | 100 | 3.6 |
| | | 14 | 7.4 | 443 | 3.45E+12 | 3836 | 49.3 | 15.2 | 9.3 | 100 | 4.7 |
| | | 30 | 7.3 | 407 | 3.37E+12 | | 21.0 | 13.4 | 10.7 | 100 | 4.3 |
| | | 60 | 7.2 | 403 | 2.75E+12 | | 65.9 | 13.3 | 10.5 | 100 | 4.3 |
| | | 90 | 7.1 | 411 | 2.53E+12 | | 41.2 | 14.6 | 11.2 | 100 | 6.2 |
| | 25° C. | 1 | 7.2 | 401 | 1.22E+12 | 4027 | 15.2 | 14.2 | 11.9 | 100 | 3.6 |
| | | 2 | 7.3 | 442 | 9.13E+11 | 2840 | 51.7 | 13.7 | 11.6 | 100 | 3.6 |
| | | 5 | 7.4 | 418 | 4.38E+12 | 2827 | 51.3 | 13.8 | 11.9 | 100 | 4.0 |
| | | 7 | 7.3 | 419 | 1.12E+12 | 2789 | 47.1 | 14.6 | 11.3 | 100 | 4.2 |
| | | 14 | 7.3 | 391 | 3.15E+12 | 3256 | 44.9 | 14.9 | 10.5 | 100 | 6.7 |
| | | 30 | 7.2 | 413 | 3.64E+12 | | 15.9 | 14.9 | 10.4 | 100 | 8.0 |

TABLE 16
Stability of 1% glycerol formulation

| | | | | | Payload | | | | | | |
|---|---|---|---|---|---|---|---|---|---|---|---|
| | | | | | | Main | | | | | |
| | | | | | | DNA | | DLS | | | SEC |
| | | Time | Vehicle | | | Species | Occupancy | | | % | % |
| Formulation | Condition | (days) | pH | Osmo | qPCR | Intensity | % Full | rH | % pd | intensity | HMW |
| 1% Glycerol | −80° C. | t0 | 8.1 | 284 | 2.43E+12 | 9437 | 52.4 | 15.1 | 9.9 | 100 | 3.9 |
| | | 1 | 8.1 | 291 | 4.23E+12 | 6522 | 51.0 | 15.1 | 9.7 | 100 | 4.0 |
| | | 2 | 8.2 | 278 | 1.08E+13 | 4865 | 54.2 | 14.3 | 11.8 | 100 | 3.9 |
| | | 5 | 8.2 | 467 | 2.59E+13 | 5844 | 52.6 | 13 | 8.4 | 100 | 3.8 |
| | | 7 | 8.2 | 400 | 4.47E+12 | 4661 | 52.1 | 15.1 | 11.4 | 100 | 4.0 |
| | | 14 | 8.2 | 337 | 2.52E+13 | 5843 | 52.9 | 15.1 | 9.7 | 100 | 5.0 |
| | | 30 | 8.2 | 264 | 1.02E+13 | | 53.2 | 15.7 | 7.8 | 100 | 3.9 |
| | | 60 | 8.0 | 284 | 5.81E+12 | | 53.5 | 13.2 | 10.0 | 100 | 4.2 |
| | | 90 | 7.9 | 278 | 1.24E+13 | | 52.0 | 14.0 | 11.6 | 100 | 4.0 |
| | 2-8° C. | 1 | 8.1 | 288 | 3.78E+12 | 5667 | 51.9 | 15.1 | 10.2 | 100 | 4.0 |
| | | 2 | 8.1 | 345 | 7.01E+12 | 6223 | 53.5 | 14.5 | 11.5 | 100 | 4.0 |
| | | 5 | 8.1 | 288 | 1.74E+13 | 6150 | 51.9 | 14.6 | 11.4 | 100 | 3.9 |
| | | 7 | 8.1 | 313 | 3.12E+12 | 5314 | 52.5 | 14.4 | 11.7 | 100 | 4.3 |
| | | 14 | 8.2 | 307 | 4.10E+13 | 5483 | 52.1 | 15.3 | 8.6 | 100 | 5.5 |
| | | 30 | 8.1 | 294 | 1.06E+13 | | 49.4 | 14.7 | 11.1 | 100 | 8.4 |
| | | 60 | 8.0 | 291 | 9.36E+12 | | 39.1 | 14.1 | 11.5 | 100 | 11.4 |
| | | 90 | 8.0 | 291 | 9.18E+12 | | 42.6 | 13.6 | 11.2 | 100 | 4.5 |
| | 25° C. | 1 | 8.1 | 287 | 3.35E+12 | 6607 | 38.2 | 15 | 10.2 | 100 | 3.9 |
| | | 2 | 8.1 | 300 | 2.93E+12 | 7244 | 52.8 | 15 | 10.3 | 100 | 3.9 |
| | | 5 | 8.2 | 291 | 1.65E+13 | 6906 | 52.4 | 15.2 | 9.5 | 100 | 4.3 |
| | | 7 | 8.2 | 315 | 2.77E+12 | 5812 | 50.3 | 15.2 | 9.7 | 100 | 4.7 |
| | | 14 | 8.2 | 302 | 2.03E+13 | 6362 | 46.1 | 15.3 | 11.2 | 100 | 7.3 |
| | | 30 | 8.1 | 302 | 9.25E+12 | | 34.5 | 15 | 10.5 | 100 | 4.4 |

TABLE 17
Stability of 2.5% glycerol formulation

| | | | | | Payload | | | | | | |
|---|---|---|---|---|---|---|---|---|---|---|---|
| | | | | | | Main | | | | | |
| | | | | | | DNA | | DLS | | | |
| | | Time | Vehicle | | | Species | Occupancy | | | % | SEC |
| Formulation | Condition | (days) | pH | Osmo | qPCR | Intensity | % Full | rH | % pd | intensity | % HMW |
| 2.5% Glycerol | −80° C. | t0 | 8.1 | 512 | 3.67E+12 | 5425 | 54.6 | 15.7 | 5.5 | 100 | 3.9 |
| | | 1 | 8.1 | 516 | 2.21E+12 | 4661 | 51.2 | 15.3 | 8.7 | 100 | 3.9 |
| | | 2 | 8.1 | 523 | 3.94E+12 | 5063 | 54.2 | 14.5 | 11.5 | 100 | 3.8 |
| | | 5 | 8.1 | 612 | 2.29E+13 | 4862 | 51.5 | 15.9 | 3.5 | 100 | 3.8 |
| | | 7 | 8.2 | 637 | 2.68E+12 | 4639 | 52.3 | 15.6 | 6.6 | 100 | 3.8 |
| | | 14 | 8.2 | 614 | 2.27E+13 | 4344 | 52.8 | 15.9 | 2.8 | 100 | 4.9 |
| | | 30 | 8.2 | 479 | 1.44E+13 | | 52.7 | 15.8 | 4.2 | 100 | 3.7 |
| | | 60 | 8.0 | 523 | 6.30E+12 | | 53.5 | 15.2 | 9.1 | 100 | 3.9 |
| | | 90 | 8.0 | 525 | 1.25E+13 | | 54.0 | 15.2 | 8.6 | 100 | 4.0 |
| | 2-8° C. | 1 | 8.1 | 511 | 5.64E+12 | 4777 | 54.6 | 14 | 11.9 | 100 | 3.9 |
| | | 2 | 8.2 | 536 | 3.94E+12 | 4568 | 53.9 | 15.5 | 7.5 | 100 | 3.9 |
| | | 5 | 8.2 | 517 | 2.15E+13 | 4591 | 52.1 | 15.6 | 6.5 | 100 | 3.9 |
| | | 7 | 8.2 | 542 | 2.95E+12 | 4770 | 51.5 | 15.9 | 13.4 | 100 | 4.1 |
| | | 14 | 8.2 | 514 | 2.88E+13 | 4368 | 50.7 | 13.8 | 11.7 | 100 | 6.7* |
| | | 30 | 8.2 | 534 | 1.30E+13 | | 42.2 | 16.5 | 11.4 | 100 | 12.8* |
| | | 60 | 8.0 | 531 | 1.02E+13 | | 35.1 | 16.1 | 4.7 | 100 | 6.9 |
| | | 90 | 8.0 | 531 | 9.81E+12 | | 35.6 | 15.6 | 6.6 | 100 | 3.4 |
| | 25° C. | 1 | 8.1 | 512 | 2.60E+12 | 4109 | 54.3 | 15.9 | 3 | 100 | 3.9 |
| | | 2 | 8.1 | 563 | 4.90E+12 | 3860 | 54.4 | 15.6 | 6.7 | 100 | 4.0 |
| | | 5 | 8.1 | 518 | 2.03E+13 | 4323 | 52.4 | 15.9 | 3.6 | 100 | 4.2 |
| | | 7 | 8.1 | 512 | 2.93E+12 | 5126 | 48.2 | 15.5 | 7.5 | 100 | 4.5 |
| | | 14 | 8.2 | 514 | 2.30E+13 | 4046 | 49.9 | 16 | 12.5 | 100 | 4.8 |
| | | 30 | 8.2 | 546 | 1.34E+13 | | 47.5 | 16.2 | 17.8 | 100 | 5.4 |

*Invalid reading due to sampling error

TABLE 18
Stability of 5.95% trehalose formulation

| | | | | | Payload | | | | | | |
|---|---|---|---|---|---|---|---|---|---|---|---|
| | | | | | | Main | | | | | |
| | | | | | | DNA | | DLS | | | SEC |
| | | Time | Vehicle | | | Species | Occupancy | | | % | % |
| Formulation | Condition | (days) | pH | Osmo | qPCR | Intensity | % Full | rH | % pd | intensity | HMW |
| 5.95% | −80° C. | t0 | 8.0 | 324 | 3.57E+12 | 4648 | 53.4 | 16.5 | 8.4 | 100 | 4.0 |
| Trehalose | | 1 | 8.1 | 330 | 2.97E+12 | 5553 | 43.9 | 15.7 | 5.3 | 100 | 4.0 |
| | | 2 | 8.1 | 341 | 3.62E+12 | 4960 | 54.9 | 15.7 | 6 | 100 | 3.9 |
| | | 5 | 8.1 | 426 | 1.57E+13 | 5454 | 52.9 | 16.6 | 9.2 | 100 | 3.9 |
| | | 7 | 8.3 | 453 | 1.44E+12 | 5481 | 52.8 | 15.7 | 6.2 | 100 | 4.0 |
| | | 14 | 8.2 | 440 | 9.29E+12 | 4950 | 53.5 | 15.9 | 2.3 | 100 | 5.2 |
| | | 30 | 8.2 | 290 | 2.14E+13 | | 51.4 | 17.2 | 11.17 | 100 | 7.1 |
| | | 60 | 8.0 | 320 | 5.31E+12 | | 47.6 | 14.2 | 11.9 | 93.2 | 8.5 |
| | | 90 | 7.9 | 315 | 1.08E+13 | | 47.5 | 17.4 | 13.0 | 100 | 8.1 |
| | 2-8° C. | 1 | 8.0 | 329 | 1.01E+12 | 5665 | 45.7 | 17.2 | 11.7 | 100 | 6.9 |
| | | 2 | 8.1 | 363 | 2.85E+12 | 6226 | 49.4 | 16.6 | 9.2 | 100 | 7.0 |
| | | 5 | 8.2 | 330 | 1.02E+13 | 5775 | 51.1 | 16.1 | 4.5 | 100 | 7.7 |
| | | 7 | 8.2 | 327 | 1.82E+12 | 6138 | 48.1 | 14.3 | 11.8 | 100 | 10.2 |
| | | 14 | 8.2 | 327 | 7.21E+12 | 6384 | 44.4 | 17.9 | 12.8 | 100 | 11.9 |
| | | 30 | 8.2 | 337 | 1.34E+13 | | 39.3 | 16.2 | 6.32 | 100 | 8.9 |
| | | 60 | 8.0 | 330 | 8.87E+12 | | 36.7 | 15.5 | 7.2 | 100 | 5.3 |
| | | 90 | 7.6 | 333 | 8.82E+12 | | 35.3 | 13.7 | 10.0 | 95.25 | 3.0 |
| | 25° C. | 1 | 8.0 | 330 | 1.58E+12 | 5422 | 41.7 | 15.7 | 5.6 | 100 | 7.2 |
| | | 2 | 8.1 | 386 | 3.10E+12 | 5304 | 50.3 | 18.2 | 20.3 | 100 | 10.5 |
| | | 5 | 8.2 | 333 | 7.52E+12 | 7266 | 41.5 | 16.6 | 9.1 | 100 | 10.9 |
| | | 7 | 8.2 | 327 | 1.20E+12 | 6889 | 38.4 | 17.9 | 13 | 100 | 8.1 |
| | | 14 | 8.2 | 328 | 4.63E+12 | 6431 | 35.0 | 15.6 | 6.3 | 100 | 4.0 |
| | | 30 | 8.0 | 340 | 8.29E+12 | | 32.5 | 17 | 14.78 | 100 | 2.3 |

TABLE 19
Stability of formulations under multiple freeze thaw cycles

| | | | | Payload | | | | | | |
|---|---|---|---|---|---|---|---|---|---|---|
| | | | | | Main | | | | | |
| | | | | | DNA | | DLS | | | |
| | Freeze | Vehicle | | | Species | Occupancy | | | % | SEC |
| Formulation | Thaw | pH | Osmo | qPCR | Intensity | % Full | rH | % pd | intensity | % HMW |
| Modified PBS | 0 | 7.4 | 305 | 1.18E+12 | 4321 | 38.2 | 14.8 | 11.5 | 100 | 3.6 |
| | 1 | 7.3 | 439 | 2.63E+12 | 6340 | 57.2 | 14.9 | 10.48 | 100 | 3.8 |
| | 2 | 6.9 | 334 | 2.46E+12 | 5436 | 49.1 | 17.3 | 32.11 | 99 | 13.0 |
| | 3 | 7.2 | 344 | 2.68E+12 | 5512 | 49.4 | 17.9 | 27.49 | 94.6 | 12.2 |
| | 4 | 7.4 | 370 | 2.40E+12 | 5323 | 40.1 | 14 | 52.38 | 60.2 | 13.0 |
| | 5 | 7.4 | 341 | 2.58E+12 | 5324 | 51.9 | 15.6 | 17.56 | 91.2 | 12.2 |
| | 6 | 7.5 | 342 | 2.00E+12 | 5371 | 46.8 | 15.2 | 9.33 | 93.7 | 11.4 |
| 1% Glycerol | 0 | 8.1 | 284 | 6.84E+12 | 5973 | 51.5 | 16.5 | 8.4 | 100 | 3.9 |
| | 1 | 8.2 | 295 | 1.65E+13 | 5411 | 51.0 | 15.1 | 9.64 | 100 | 4.0 |
| | 2 | 8.2 | 269 | 1.45E+13 | 5632 | 52.8 | 14.9 | 10.62 | 100 | 3.9 |
| | 3 | 8.0 | 283 | 1.35E+13 | 5954 | 52.2 | 14.5 | 15.1 | 96.2 | 4.1 |
| | 4 | 8.1 | 295 | 1.32E+13 | 6108 | 51.3 | 15.1 | 9.88 | 100 | 4.1 |
| | 5 | 8.1 | 548 | 1.47E+13 | 6283 | 51.7 | 14.7 | 11.06 | 100 | 4.3 |
| | 6 | 8.1 | 269 | 1.22E+13 | 6833 | 51.7 | 14 | 11.87 | 100 | 3.9 |
| 2.5% Glycerol | 0 | 8.1 | 512 | 7.58E+12 | 8712 | 50.1 | 15.7 | 5.5 | 100 | 3.9 |
| | 1 | 8.2 | 553 | 1.51E+13 | 9004 | 51.2 | 16.3 | 7.38 | 100 | 3.9 |
| | 2 | 8.2 | 484 | 1.30E+13 | 9476 | 52.2 | 15.3 | 8.88 | 100 | 4.0 |
| | 3 | 8.1 | 512 | 1.60E+13 | 9392 | 51.2 | 16 | 15.1 | 100 | 4.0 |
| | 4 | 8.1 | 505 | 1.44E+13 | 9433 | 51.6 | 15.4 | 8.08 | 100 | 4.1 |
| | 5 | 8.1 | 490 | 1.57E+13 | 9145 | 51.3 | 14.3 | 11.8 | 100 | 4.2 |
| | 6 | 8.2 | 490 | 1.51E+13 | 10967 | 51.7 | 16 | 10.58 | 100 | 4.0 |
| 5.95% | 0 | 8.2 | 311 | 5.22E+12 | 8962 | 52.6 | 15.1 | 9.9 | 100 | 4.0 |
| Trehalose | 1 | 8.2 | 318 | 1.36E+13 | 12692 | 43.9 | 18 | 12.1 | 100 | 4.0 |
| | 2 | 8.3 | 307 | 1.45E+13 | 11357 | 48.5 | 17.3 | 11.46 | 100 | 7.7 |
| | 3 | 8.1 | 326 | 1.36E+13 | 11217 | 45.6 | 16 | 0.86 | 100 | 12.7 |
| | 4 | 8.0 | 324 | 1.34E+13 | 8817 | 46.4 | 17.4 | 15.08 | 100 | 14.6 |
| | 5 | 8.1 | 311 | 1.49E+13 | 8326 | 46.7 | 17.1 | 10.97 | 100 | 13.5 |
| | 6 | 8.2 | 318 | 2.35E+13 | 9011 | 47.6 | 15.8 | 4.66 | 100 | 10.4 |

### Example 10. Dose-Response Evaluation of TTD-001 in Non-Human Primates (NHPs)

This Example investigates the minimal dose of an AAV particle comprising a TTD-001 capsid variant (SEQ ID NO: 3623 (DNA) and 3636 (amino acid), comprising SEQ ID NO: 1725 or 3648)) that is sufficient to achieve near-physiological expression of a payload, e.g., a single stranded payload, in the central nervous system of adult cynomolgus macaques (Macaca fascicularis) via intravenous systemic delivery.

AAV particles comprising the TTD-001 capsid variant comprising a single stranded viral genome encoding a hemagglutinin (HA)-tagged NHP protein under the control of a ubiquitous CBA promoter were injected intravenously into adult male NHPs (cynomolgus macaque) (n=3, 5-7 years of age) at various doses spanning a 30-fold range, which included 6.7e11 VG/kg, 2e12 VG/kg, 6.7e12 VG/kg, and 2e13 VG·kg. The in-life period was 28 days and then various CNS and peripheral tissues were collected for measuring transgene mRNA expression by RT-qPCR, viral DNA levels by ddPCR, transgene protein expression by ELISA, and biodistribution by immunohistochemistry (staining with an anti-HA antibody).

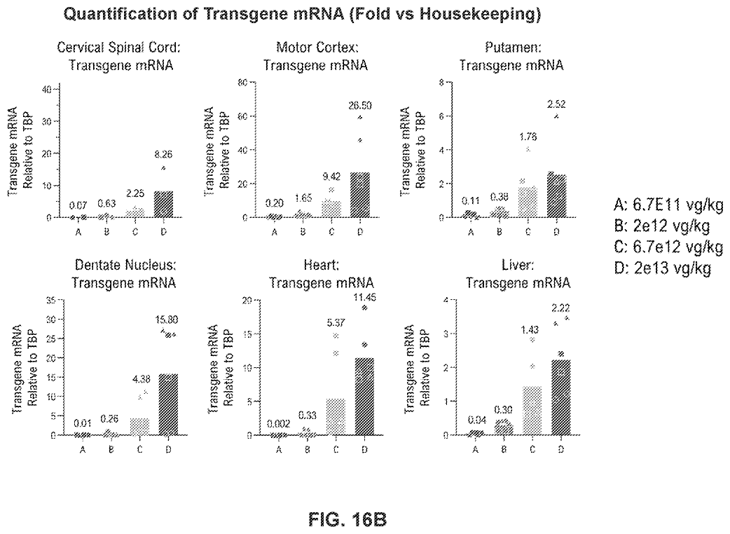

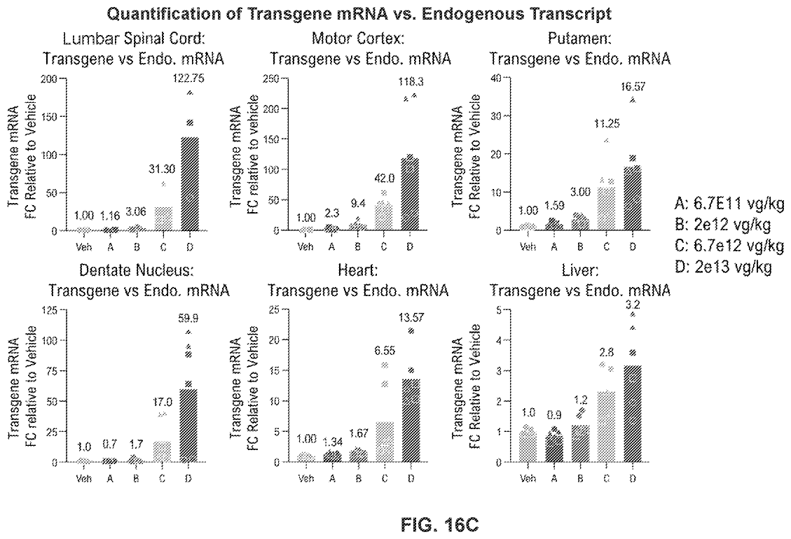

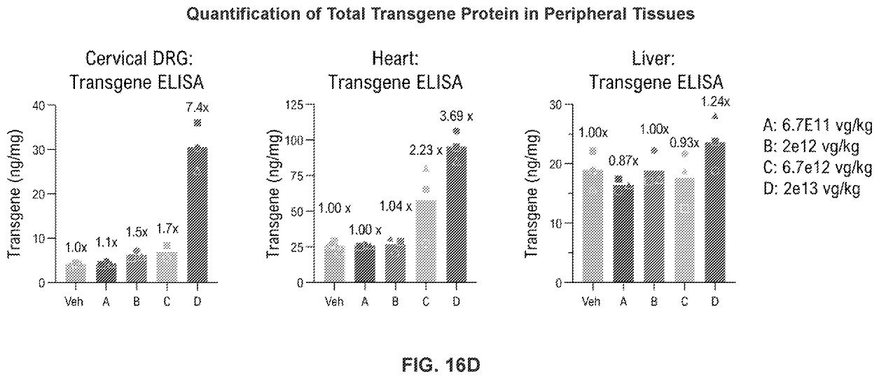

Widespread transgene expression was detected in the spinal cord and the brain the NHPs at doses of 2e12 VG/kg and above, especially in the putamen, thalamus, globus pallidus and brainstem (Tables 21-23 and FIGS. 16A-16D). Viral DNA and mRNA were readily detectable in all NHPs and showed a consistent dose response (Table 21, Table 22, and FIGS. 16A-16D).

More specifically, in the brain, dose-dependent distribution of the AAV particles comprising the TTD-001 capsid was observed in the cortical regions (frontal, motor, and somatosensory), caudate, putamen, thalamus, substantia nigra, globus pallidus, hippocampus, amygdala, hypothalamus, cerebellar cortex, and dentate nucleus. Additionally, for each dose administered, there was comparable distribution of the AAV particles comprising the TTD-001 capsid in each brain region, including the cortex as well as the deeper brain regions such as the caudate, putamen, thalamus, substantia nigra, globus pallidus, hippocampus, amygdala, hypothalamus, and dentate nucleus (Table 21; FIG. 16A).

With respect to the spinal cord, dose-dependent distribution of the AAV particles comprising the TTD-001 capsid was observed in the cervical, thoracic, and lumbar spinal cord regions and the relative distribution across all these regions was similar for each dosing group. As shown in Table 21 and FIG. 16A, low biodistribution was measured in the DRG, but a dose-dependent distribution of the AAV particles comprising the TTD-1 capsid was observed in the cervical, thoracic, and lumbar DRG regions and the relative distribution across all DRG regions was similar for each dosing group.

With respect to the peripheral tissues, a dose-dependent distribution of the AAV particles comprising the TTD-001 capsid was observed in the liver, hear, and the vastus lateralis (muscle) (FIG. 16A and Table 21).

TABLE 21
Quantification of viral genomes (biodistribution) by ddPCR following intravenous
administration of various doses of AAV particles comprising a TTD-001 capsid

| | Quantification of viral genomes in the Brain (VG/diploid cell) | | | | | | |
|---|---|---|---|---|---|---|---|
| Dose | Frontal | Motor | Somatosensory | | | | Dentate |
| (VG/kg) | Cortex | Cortex | Cortex | Caudate | Putamen | Thalamus | Nucleus |
| 6.7e11 | 0.01 | 0.11 | 0.07 | 0.16 | 0.13 | 0.17 | 0.03 |
| 2e12 | 2.0 | 1.72 | 0.92 | 1.27 | 0.89 | 1.16 | 0.42 |
| 6.7e12 | 4.5 | 4.20 | 3.44 | 3.44 | 2.74 | 3.04 | 2.27 |
| 2e13 | 6.7 | 7.50 | 3.04 | 6.32 | 4.94 | 6.34 | 4.14 |

| Dose | Substantia | Globus | | | | |
|---|---|---|---|---|---|---|
| (VG/kg) | Nigra | Pallidus | Hippocampus | Amygdala | Hypothalamus | Cerebellar Cortex |
| 6.7ell | 0.12 | 0.10 | 0.1 | 0.16 | 0.14 | 0.02 |
| 2e12 | 1.12 | 1.28 | 2.1 | 2.83 | 1.77 | 0.13 |
| 6.7e12 | 2.33 | 2.47 | 2.6 | 4.38 | 2.37 | 0.53 |
| 2e13 | 5.81 | 4.43 | 5.5 | 6.13 | 4.63 | 4.18 |

| | Quantification of viral genomes in the Spinal Cord and DRG (VG/diploid cell) | | | | | |
|---|---|---|---|---|---|---|
| | Cervical | Thoracic | | | | |
| Dose | Spinal | Spinal | Lumbar Spinal | Cervical | | |
| (VG/kg) | Cord | Cord | Cord | DRG | Thoracic DRG | Lumbar DRG |
| 6.7ell | 0.06 | 0.03 | 0.05 | 0.002 | 0.003 | 0.007 |
| 2e12 | 0.74 | 0.42 | 0.64 | 0.007 | 0.006 | 0.019 |
| 6.7e12 | 1.35 | 0.94 | 1.58 | 0.015 | 0.022 | 0.033 |
| 2e13 | 5.35 | 3.48 | 6.48 | 0.060 | 0.079 | 0.070 |

| Dose | Quantification of viral genomes in the peripheral tissues (VG/diploid cell) | | | |
|---|---|---|---|---|
| (VG/kg) | Heart | Liver | Kidney | Vastus lateralis (muscle) |
| 6.7ell | 0.11 | 1.2 | 0.04 | 0.002 |
| 2e12 | 0.10 | 12.4 | 0.71 | 0.02 |
| 6.7e12 | 0.30 | 68.5 | 1.18 | 0.06 |
| 2e13 | 0.56 | 132.3 | 0.84 | 0.15 |

Additionally, dose-dependent transgene mRNA expression by the AAV particles comprising the TTD-001 capsid was observed in the brain, spinal cord, DRG, and peripheral tissues (FIG. 16B; Table 22). The lowest dose of the AAV particles comprising the TTD-001 capsid protein resulted in higher transgene mRNA and protein expression than a 30-fold higher dose of wild-type AAV9. Comparison of the transgene mRNA with the matching endogenous transcript indicated that a dose of 2e12 VG/kg was sufficient to achieve supra-physiological levels in the central nervous system (CNS), while showing low transduction in the liver and the dorsal root ganglia (DRG) (Table 22; FIG. 16C).

TABLE 22
Quantification of transgene mRNA by RT-qPCR following intravenous administration
of various doses of AAV particles comprising a TTD-001 capsid
Transgene mRNA relative to housekeeping gene

| | Dose (VG/kg) | | | |
|---|---|---|---|---|
| Tissue | 6.7e11 VG/kg | 2e12 VG/kg | 6.7e12 VG/kg | 2e13 VG/kg |
| Frontal Cortex | 0.02 | 0.29 | 3.42 | 4.51 |
| Motor Cortex | 0.2 | 1.65 | 9.42 | 26.5 |
| Putamen | 0.11 | 0.38 | 1.76 | 2.52 |
| Dentate Nucleus | 0.01 | 0.26 | 4.38 | 15.8 |
| Cervical Spinal Cord | 0.07 | 0.63 | 2.25 | 8.26 |
| Thoracic Spinal Cord | 0.06 | 0.72 | 2.00 | 5.37 |
| Lumbar Spinal Cord | 0.10 | 0.83 | 6.71 | 27.22 |
| Cervical DRG | 0.02 | 0.29 | 0.99 | 3.58 |
| Thoracic DRG | 0.01 | 0.28 | 1.08 | 4.36 |
| Lumbar DRG | 0.02 | 0.56 | 2.21 | 4.44 |
| Heart | 0.002 | 0.33 | 5.37 | 11.45 |
| Liver | 0.04 | 0.3 | 1.43 | 2.22 |

Transgene mRNA vs. endogenous transcript (fold change relative to vehicle control)

| | Dose (VG/kg) | | | | |
|---|---|---|---|---|---|
| Tissue | Vehicle | 6.7e11 VG/kg | 2e12 VG/kg | 6.7e12 VG/kg | 2e13 VG/kg |
| Frontal Cortex | 1.0 | 0.97 | 2.04 | 12.79 | 17.04 |
| Motor Cortex | 1.0 | 2.3 | 9.4 | 42.0 | 118.3 |
| Putamen | 1.0 | 1.59 | 3.00 | 11.25 | 16.57 |
| Dentate Nucleus | 1.0 | 0.7 | 1.7 | 17.0 | 59.9 |
| Cervical Spinal Cord | 1.0 | 1.18 | 2.65 | 12.57 | 43.16 |
| Thoracic Spinal Cord | 1.0 | 1.07 | 2.26 | 10.61 | 27.96 |
| Lumbar Spinal Cord | 1.0 | 1.16 | 3.06 | 31.30 | 122.75 |
| Cervical DRG | 1.0 | 1.0 | 1.63 | 3.24 | 9.09 |
| Thoracic DRG | 1.0 | 0.98 | 1.4 | 3.02 | 7.72 |
| Lumbar DRG | 1.0 | 1.05 | 2.3 | 4.78 | 11.72 |
| Heart | 1.0 | 1.34 | 1.67 | 6.55 | 13.57 |
| Liver | 1.0 | 0.9 | 1.2 | 2.8 | 3.2 |

TABLE 23
Quantification of total transgene protein expression in the peripheral
tissues by ELISA following intravenous administration of various
doses of AAV particles comprising a TTD-001 capsid

| | Cervical DRG | Heart | Liver |
|---|---|---|---|
| | (Transgene ng/mL | (Transgene ng/mL | (Transgene ng/mL |
| Dose | relative | relative | relative |
| (VG/kg) | to vehicle) | to vehicle) | to vehicle) |
| Vehicle | 1.0 | 1.0 | 1.0 |
| 6.7e11 | 1.1 | 1.0 | 0.87 |
| 2e12 | 1.5 | 1.04 | 1.00 |
| 6.7e12 | 1.7 | 2.23 | 0.93 |
| 2e13 | 7.4 | 3.69 | 1.24 |

By immunohistochemistry (JHC), widespread transduction by AAV particles comprising the TTD-001 capsid variant was observed in multiple brain regions of the NHPs as compared to AAV9 at all doses administered, particularly at the medium to high doses (2e12 VG/kg, 6.7e12 VG/kg, and 2e13 VG/kg). By JHC, dose dependent expression of AAV particles comprising the TTD-001 capsid variant was observed in the brain, specifically in the temporal cortex, caudate, putamen, thalamus, substantia nigra, hippocampus, and cerebellar. Morphologically, transgene expression was observed in the neuronal cell body and the neuropil from neurons in these brain regions, including the Purkinje neurons in the cerebellar cortex and the neurons deep in the cerebellar nuclei. In the brain stem, the transgene expression was observed in various structures including the gracile-nuclei, cuneate-nuclei, and the Inferior Olivary complex.

In the spinal cord of the NHPs, dose dependent transduction was also observed in the cervical, lumbar, and thoracic regions when measured by IHC, with the most intense and widespread staining occurring at the 6.7e12 VG/kg and 2e13 VG/kg doses. Substantial staining of the motor neurons in the spinal cord was also observed at the lower dose of 2e12 VG/kg. Furthermore, the cellular tropism of the TTD-001 capsid in the spinal cord appeared to be largely neuronal and neuropil at all doses in all regions (e.g., cervical, thoracic, and lumbar) investigated.

In the DRG of the NHPs, dose dependent transduction was also observed in the cervical, lumbar, and thoracic regions, with the most staining occurring at the 6.7e12 VG/kg and 2e13 VG/kg doses. The lower dose of 2e12 VG/kg showed significantly less staining and was comparable to particles comprising an AAV9 capsid that were administered at a higher dose of 2e13 VG/kg. The cellular tropism of the TTD-001 capsid in the DRG appeared to be largely neuronal at all doses in all regions investigated.

Transduction of AAV particles comprising the TTD-001 capsid variant was also measured by IHC in various peripheral tissues of the NHPs. In the liver, the transduction observed was more variable but appeared to follow a dose-dependent trend and appeared to be lower than by particles comprising an AAV9 capsid that were administered at a dose of 2e13 VG/kg. Minimal staining was observed in the quadriceps at all doses tested. In the heart, a dose-dependent trend in transduction was also observed.

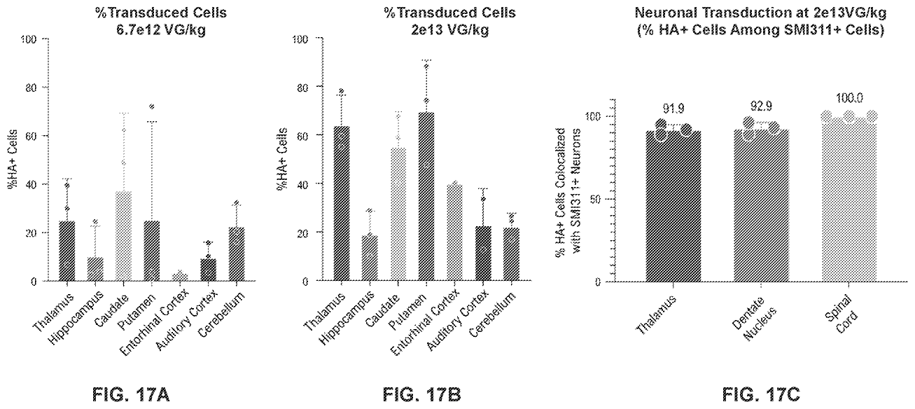

Additionally, the staining of various cells in the brain and/or spinal cord following transduction with the AAV particles comprising the TTD-001 capsid at the doses investigated was quantified. As shown in FIG. 17B, a dose of 2e13 VG/kg was sufficient to transduce >40% of total cells in highly permissive brain regions (thalamus, caudate, putamen) and >20% total cells in less permissive regions (entorhinal cortex, auditory cortex, hippocampus). Even at a lower dose of 6.7e12, this was sufficient to transduce >20% of cells in the thalamus, caudate, putamen, and cerebellum (FIG. 17A). As shown in FIG. 17C, the dose of 2e13 VG/kg also resulted in transduction of >90% SMI311-positive neurons in the thalamus, dentate and spinal cord.

Together, these data demonstrate that variant AAV capsids, including TTD-001, can achieve a large improvement of their therapeutic index by retaining strong efficacy at low dose.

We claim:

1. An AAV capsid variant, comprising an amino sequence comprising the following formula: [N1]-[N2], wherein:
(i) [N1] comprises X1, X2, X3, X4, and X5, wherein:
(a) position X1 is: P, Q, A, H, K, L, R, S, or T;
(b) position X2 is: L, I, V, H, or R;
(c) position X3 is: N, D, I, K, or Y;
(d) position X4 is: G, A, C, R, or S; and
(e) position X5 is: A, S, T, G, C, D, N, Q, V, or Y; and
(ii) [N2] comprises the amino acid sequence of VHLY (SEQ ID NO: 4680), VHIY (SEQ ID NO: 4681), VHVY (SEQ ID NO: 4682), or VHHY (SEQ ID NO: 4683); or a substitution, e.g., a conservative substitution, of any of the aforesaid amino acids in (i) and/or (ii);
wherein [N1]-[N2] is present from N-terminus to C-terminus, immediately subsequent to position 586, numbered according to any one of SEQ ID NO: 5, 8, or 3636; and
wherein the AAV capsid variant further comprises one, two, or all of (a)-(c):
(a) the amino acid L, T, V, R, S, A, C, I, K, M, N, P, or Q at position 600, the amino acid S, G, A, T, M, V, Q, L, H, I, K, N, P, R, or Y at position 601, and the amino P, W, S, G, A, Q, L, M, K, C, E, F, H, R, T, V, or Y at position 602, numbered according to the amino acid sequence of SEQ ID NO: 5, 8, 3636, wherein the AAV capsid variant does not comprise the amino acid sequence TGW at position 600-602, numbered according to SEQ ID NO: 5, 8, or 3636;
(b) the amino acid V, D, F, G, L, A, E, or I at position 603, the amino acid K, P, Q, R, H, E, or L at position 604, and the amino acid N, T, K, H, D, Y, S, I, or P, numbered according to the amino acid sequence of SEQ ID NO: 5, 8, or 3636, wherein the AAV capsid variant does not comprise the amino acid sequence VQN at position 603-605, numbered according to SEQ ID NO: 5, 8, or 3636; and/or
(c) one, two, three, four, or all of an amino acid other then P at position X1, an amino acid other than L at position X2, an amino acid other than N at position X3, an amino acid other than G at position X4, or an amino acid other than A at position X5.

2. The AAV capsid variant of claim 1, wherein [N1] comprises PLNGA (SEQ ID NO: 3679), SLNGA (SEQ ID NO: 4684), QLNGA (SEQ ID NO: 4685), ALNGA (SEQ ID NO: 4686), PLNGS (SEQ ID NO: 4687), PVNGA (SEQ ID NO: 4688), PLNGG (SEQ ID NO: 4689), PLNGT (SEQ ID NO: 4690), PLDGA (SEQ ID NO: 4691), QLNGS (SEQ ID NO: 4692), PLNGN (SEQ ID NO: 4693), SLDGA (SEQ ID NO: 4694), HLNGA (SEQ ID NO: 4695), ALNGT (SEQ ID NO: 4696), PINGA (SEQ ID NO: 4697), ALDGA (SEQ ID NO: 4698), PLNCA (SEQ ID NO: 4699), PLNGQ (SEQ ID NO: 4700), PLDSA (SEQ ID NO: 4701), RLDGA (SEQ ID NO: 4702), QLNGN (SEQ ID NO: 4703), PLNGY (SEQ ID NO: 4704), PLDSS (SEQ ID NO: 4705), PLNGC (SEQ ID NO: 4706), PLYGA (SEQ ID NO: 4707), TLNGA (SEQ ID NO: 4708), PVDGA (SEQ ID NO: 4709), PLKGA (SEQ ID NO: 4710), PLNGD (SEQ ID NO: 4711), KLDGA (SEQ ID NO: 4712), PHNGA (SEQ ID NO: 4713), PLNGV (SEQ ID NO: 4714), PLNAA (SEQ ID NO: 4715), QLNGY (SEQ ID NO: 4716), PLDGS (SEQ ID NO: 4717), LLNGA (SEQ ID NO: 4718), PLNRA (SEQ ID NO: 4719), PLIGA (SEQ ID NO: 4720), PRNGA (SEQ ID NO: 4721), or ALNGS (SEQ ID NO: 4722), or a substitution, e.g., a conservative substitution, of any of the aforesaid amino acid sequences.

3. The AAV capsid variant of claim 1 or 2, wherein [N1]-[N2] comprises:
(i)
(SEQ ID NO: 3648)
PLNGAVHLY,
(SEQ ID NO: 4780)
ALDGAVHLY,
(SEQ ID NO: 4781)
ALNGAVHLY,
(SEQ ID NO: 4782)
PINGAVHLY,
(SEQ ID NO: 4783)
PLDGAVHLY,
(SEQ ID NO: 4784)
PLDSAVHLY,
(SEQ ID NO: 4785)
PLDSSVHLY,
(SEQ ID NO: 4786)
PLNGGVHLY,
(SEQ ID NO: 4787)
PLNGNVHLY,
(SEQ ID NO: 4788)
PLNGSVHLY,
(SEQ ID NO: 4789)
PLNGTVHLY,
(SEQ ID NO: 4790)
QLNGAVHLY,
(SEQ ID NO: 4791)
SLDGAVHLY,
(SEQ ID NO: 4792)
SLNGAVHLY,
(SEQ ID NO: 4793)
TLNGAVHLY,
(SEQ ID NO: 4794)
PLNGAVHIY,
(SEQ ID NO: 4795)
PLDGAVHVY,
or
(SEQ ID NO: 4796)
PLNGAVHHY;
(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four substitutions (e.g., conservative substitutions), relative to any of the amino acid sequences in (i); or
(iv) an amino acid sequence comprising at least one, two, or three but no more than four different amino acids, relative to any one of the amino acid sequences in (i)
4. The AAV capsid variant of any one of claims 1-3, which further comprises [N3], wherein [N3] comprises X6, X7, X8, and X9, wherein:
(a) position X6 is: A, D, S, or T;
(b) position X7 is: Q, K, H, L, P, or R;
(c) position X8 is: A, P, E, or R; and
(d) position X9 is: Q, H, K, or P; or a substitution, e.g., a conservative substitution, of any of the aforesaid amino acids in (a)-(d).

5. The AAV capsid variant of claim 4, wherein [N3] comprises AQAQ (SEQ ID NO: 4737), SQAQ (SEQ ID NO: 4738), AQPQ (SEQ ID NO: 4739), AQSQ (SEQ ID NO: 4740), AKAQ (SEQ ID NO: 4741), AHAQ (SEQ ID NO: 4742), AQAP (SEQ ID NO: 4743), DQAQ (SEQ ID NO: 4744), APAQ (SEQ ID NO: 4745), AQAK (SEQ ID NO: 4746), AQAH (SEQ ID NO: 4747), AQEQ (SEQ ID NO: 4748), ALAQ (SEQ ID NO: 4749), ARAQ (SEQ ID NO: 4750), or TQAQ (SEQ ID NO: 4751), or a substitution, e.g., a conservative substitution, of any of the aforesaid amino acid sequences.

6. The AAV capsid variant of any one of claims 1-5, which further comprises [N4], wherein [N4] comprises X10, X11, and X12, wherein:
(a) position X10 is: L, T, V, R, S, A, C, I, K, M, N, P, or Q;
(b) position X11 is: S, G, A, T, M, V, Q, L, H, I, K, N, P, R, or Y; and
(c) position X12 is: P, W, S, G, A, Q, L, M, K, C, E, F, H, R, T, V, or Y; or a substitution, e.g., a conservative substitution, of any of the aforesaid amino acids in (a)-(c).

7. The AAV capsid variant of claim 6, wherein [N4] comprises:
(i) LSP, TGW, TGL, TGS, TGG, TAW, TGR, TAS, LSS, TSS, SSL, SSS, TLS, TVS, VSS, TSP, VSP, TMS, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW, or a substitution, e.g., a conservative substitution, of any of the aforesaid amino acid sequences;
(ii) LSP; or
(iii) TGW.

8. The AAV capsid variant of any one of claims 1-7, which further comprises [N5], wherein [N5] comprises X13, X14, and X15, wherein:
(a) position X13 is: V, D, F, G, L, A, E, or I;
(b) position X14 is: K, P Q, R, H, E, or L; and
(c) position X15 is: N, T, K, H, D, Y, S, I, or P; or a substitution, e.g., a conservative substitution, of any of the aforesaid amino acids in (a)-(c).

9. The AAV capsid variant of claim 8, wherein [N5] comprises:
(i) VQN, VPN, VKN, VQT, VQK, DQN, VQH, GQN, VQI, VHN, FQN, LQN, VLN, VRN, VQS, VQY, AQN, VEN, VQD, IQN, VKK, DKN, VKT, VQP, EQN, GQT, FQK, GHN, or VPH, or a substitution, e.g., a conservative substitution, of any of the aforesaid amino acid sequences;
(ii) VKN, VPN, VEN, or VHN; or
(iii) VQN.

10. The AAV capsid variant of claim 8 or 9, wherein [N1]-[N2]-[N3]-[N4]-[N5] comprises:
(i) the amino acid sequence of any of SEQ ID NOs: 139-475 or 477-1138;
(ii) the amino acid sequence of PLNGAVHLYAQAQLSPVKN (SEQ ID NO: 566);
(iii) the amino acid sequence of PLNGAVHLYAQAQTGWVPN (SEQ ID NO: 314);
(iv) the amino acid sequence of any of SEQ ID NOs: 14-17, 40-136, 314, 325, 491, 499, 529, 558, 566, 576, 603, 610, 625, 631, 648, 649, 700, 703, 720, 755, 763, 765, 771, 791, 804, 816, 818, 819, 828, 859, 864, 871, 885, 946, 960, 966, 978, 979, 1016, 1033, 1032, 1037, 1058, 1081, 1100, 1122, or 1174-1193;
(v) an amino acid sequence comprising any portion of an amino acid sequence in (i)-(iv), e.g., any 2, 3, 4, 5, 6, 7, 8, 9, 10, 11, 12, 13, 14, 15, 16, 17, or 18 amino acids, e.g., consecutive amino acids, thereof;
(vi) an amino acid sequence comprising at least one, two, or three but no more than four substitutions (e.g., conservative substitutions), relative to any of the amino acid sequences in (i)-(iv); or
(vii) an amino acid sequence comprising at least one, two, or three but no more than four different amino acids, relative to any one of the amino acid sequences in (i)-(iv).

11. The AAV capsid variant of any one of claims 8-10, which comprises from N-terminus to C-terminus [N1]-[N2]-[N3]-[N4]-[N5], wherein:
(i) [N1] is present immediately subsequent to position 586, and replaces positions 587 and 588 (e.g., A587 and Q588), numbered according to SEQ ID NO: 138;
(ii) [N2] is present immediately subsequent to [N1];
(iii) [N3] is present immediately subsequent to position 588, and replaces positions 589-592 (e.g., A589, Q590, A591, Q592), numbered according to the amino acid sequence of SEQ ID NO: 138;
(iv) [N4] is present immediately subsequent to position 592, and replaces positions 593-595 (e.g., T593, G594, W595), numbered according to the amino acid sequence of SEQ ID NO: 138;
(v) [N5] is present immediately subsequent to position 595, and replaces positions 596-598 (e.g., V596, Q597, N598), numbered according to the amino acid sequence of SEQ ID NO: 138; and/or
(vi) [N1]-[N2]-[N3]-[N4]-[N5] is present immediately subsequent to position 586 and replaces positions 587-598 (e.g., A587, Q588, A589, Q590, A591, Q592, T593, G594, W595, V596, Q597, N598), numbered according to SEQ ID NO: 138.

12. The AAV capsid variant of any one of claims 8-11, wherein:
(i) the AAV capsid variant comprises an amino acid other than A at position 587 and/or an amino acid other than Q at position 588, numbered according to SEQ ID NO: 138;
(ii) [N1] corresponds to positions 587-591 of SEQ ID NO: 5, 8, or 3636;
(iii) [N2] corresponds to positions 592 to 595 of SEQ ID NO: 5, 8, or 3636;
(iv) [N3] corresponds to positions 596-599 of SEQ ID NO: 5, 8, or 3636;
(v) [N4] corresponds to positions 600-602 of SEQ ID NO: 5, 8, or 3636;
(vi) [N5] corresponds to positions 603-605 of SEQ ID NO: 5, 8, or 3636; and/or
(vii) [N1]-[N2]-[N3]-[N4]-[N5] corresponds to positions 587-605 of SEQ ID NO: 5, 8, or 3636.

13. An AAV capsid variant comprising [A][B], wherein [A] comprises the amino acid sequence of PLNGA (SEQ ID NO: 3679), and [B] comprises X1, X2, X3, X4, wherein:
(i) X1 is: V, I, L, A, F, D, or G;
(ii) X2 is: H, N, Q, P, D, L, R, or Y;
(iii) X3 is: L, H, I, R, or V; and
(iv) X4 is Y; or wherein the AAV capsid variant comprises a substitution e.g., a conservative substitution, of any of the aforesaid amino acids in (i)-(iv);
wherein [N1]-[N2] is present from N-terminus to C-terminus, immediately subsequent to position 586, numbered according to any one of SEQ ID NO: 5, 8, 138, or 3636; and
wherein the AAV capsid variant further comprises one, two, or all of (a)-(c):
(a) the amino acid L, T, V, S, R, I, A, N, C, Q, M, P, or K at position 600, the amino acid S, G, T, M, A, K, Q, V, I, R, N, P, L, H, or Y at position 601, and/or the amino acid P, W, K, Q, S, C, A, G, N, T, R, V, M, H, L, E, F, or Y at position 602, numbered according to the amino acid sequence of SEQ ID NO: 5, 8, 3636, wherein the AAV capsid variant does not comprise the amino acid sequence TGW at position 600-602, numbered according to SEQ ID NO: 5, 8, or 3636;
(b) the amino acid V, D, F, A, E, L, G, or I at position 603, the amino acid K, P, Q, R, L, H, or E at position 604, and/or the amino acid N, H, S, T, P, K, I, D, or Y at position 605, numbered according to the amino acid sequence of SEQ ID NO: 5, 8, or 3636, wherein the AAV capsid variant does not comprise the amino acid sequence VQN at position 603-605, numbered according to SEQ ID NO: 5, 8, or 3636; and/or
(c) one, two, three, or all of an amino acid other than V at position X1, an amino acid other than H at position X2, an amino acid other than L at position X3, and/or Y at position X4.

14. The AAV capsid variant of claim 13, wherein [B] is or comprises:
(i) VHLY (SEQ ID NO: 4680), VHHY (SEQ ID NO: 4683), VHIY (SEQ ID NO: 4681), VNLY (SEQ ID NO: 4724), VQLY (SEQ ID NO: 4729), IHLY (SEQ ID NO: 4730), LHLY (SEQ ID NO: 4727), VPLY (SEQ ID NO: 4723), VDLY (SEQ ID NO: 4731), AHLY (SEQ ID NO: 4732), VHRY (SEQ ID NO: 4725), FHLY (SEQ ID NO: 4726), DHLY (SEQ ID NO: 4728), VLLY (SEQ ID NO: 4733), GHLY (SEQ ID NO: 4734), VRLY (SEQ ID NO: 4735), VHVY (SEQ ID NO: 4682), or VYLY (SEQ ID NO: 4736), or a substitution, e.g., a conservative substitution, of any of the aforesaid amino acid sequences; or
(ii) VHLY (SEQ ID NO: 4680).

15. The AAV capsid variant of claim 13 or 14, wherein [A][B] comprises:
(i)
(SEQ ID NO: 3648)
PLNGAVHLY,
(SEQ ID NO: 4796)
PLNGAVHHY,
(SEQ ID NO: 4794)
PLNGAVHIY,
(SEQ ID NO: 5123)
PLNGAVNLY,
(SEQ ID NO: 5124)
PLNGAVQLY,
(SEQ ID NO: 5125)
PLNGAIHLY,
(SEQ ID NO: 5126)
PLNGALHLY,
(SEQ ID NO: 5127)
PLNGAVPLY,
(SEQ ID NO: 5128)
PLNGAVDLY,
(SEQ ID NO: 5129)
PLNGAAHLY,
(SEQ ID NO: 5130)
PLNGAVHRY,
(SEQ ID NO: 5131)
PLNGAFHLY,
(SEQ ID NO: 5132)
PLNGADHLY,
(SEQ ID NO: 5133)
PLNGAVLLY,
(SEQ ID NO: 5134)
PLNGAGHLY,
(SEQ ID NO: 5135)
PLNGAVRLY,
(SEQ ID NO: 5136)
PLNGAVHVY,
or
(SEQ ID NO: 5137)
PLNGAVYLY;
(ii)
(SEQ ID NO: 3648)
PLNGAVHLY;
(iii) an amino acid sequence comprising any portion of an amino acid sequence in (i) or (ii), e.g., any 2, 3, 4, 5, 6, 7, or 8 amino acids, e.g., consecutive amino acids, thereof;
(iv) an amino acid sequence comprising at least one, two, or three but no more than four substitutions (e.g., conservative substitutions), relative to any of the amino acid sequences in (i) or (ii); or
(v) an amino acid sequence comprising at least one, two, or three but no more than four different amino acids, relative to any one of the amino acid sequences in (i) or (ii).

16. The AAV capsid variant of any one of claims 13-15, which further comprises [C], wherein [C] comprises X4, X5, X6, and X7, wherein:
(a) position X4 is: A, D, S, or T;
(b) position X5 is: Q, K, H, L, P, or R;
(c) position X6 is: A, P, or E; and
(d) position X7 is: Q, H, K, or P;
or a substitution, e.g., a conservative substitution, of any of the aforesaid amino acids in (a)-(d).

17. The AAV capsid variant of claim 16, wherein [C] is or comprises:
(i) AQAQ (SEQ ID NO: 4737), AQPQ (SEQ ID NO: 4739), AKAQ (SEQ ID NO: 4741), DQAQ (SEQ ID NO: 4744), SQAQ (SEQ ID NO: 4738), AHAQ (SEQ ID NO: 4742), AQEQ (SEQ ID NO: 4748), AQAK (SEQ ID NO: 4746), ALAQ (SEQ ID NO: 4749), APAQ (SEQ ID NO: 4745), ARAQ (SEQ ID NO: 4750), AQAH (SEQ ID NO: 4747), AQAP (SEQ ID NO: 4743), or TQAQ (SEQ ID NO: 4751), or a substitution, e.g., a conservative substitution, of any of the aforesaid amino acid sequences; or
(ii) AQAQ (SEQ ID NO: 4737).

18. The AAV capsid variant of any one of claims 1-17, which further comprises:
(i) the amino acid L at position 593 numbered according to SEQ ID NO: 138 or at position 600 numbered according to SEQ ID NO: 5, 8, or 3636; the amino acid S at position 594 numbered according to SEQ ID NO: 138, or at position 601 numbered according to SEQ ID NO: 5, 8, or 3636; and the amino acid P at position 595 numbered according to SEQ ID NO: 138 or at position 602 numbered according to SEQ ID NO: 5, 8, or 3636; or
(ii) the amino acid T at position 593 numbered according to SEQ ID NO: 138 or at position 600 numbered according to SEQ ID NO: 5, 8, or 3636; the amino acid G at position 594 numbered according to SEQ ID NO: 138, or at position 601 numbered according to SEQ ID NO: 5, 8, or 3636; and the amino acid W at position 595 numbered according to SEQ ID NO: 138 or at position 602 numbered according to SEQ ID NO: 5, 8, or 3636.

19. The AAV capsid variant of any one of claims 13-18, which further comprises [D], wherein [D] comprises X8, X9, and X10, wherein:
(a) position X8 is: L, T, V, S, R, I, A, N, C, Q, M, P, or K;
(b) position X9 is: S, G, T, M, A, K, Q, V, I, R, N, P, L, H, or Y; and
(c) position X10 is: P, W, K, Q, S, C, A, G, N, T, R, V, M, H, L, E, F, or Y; or a substitution, e.g., a conservative substitution, of any of the aforesaid amino acids in (a)-(c).

20. The AAV capsid variant of any one of claims 13-19, wherein [D] is or comprises:
(i) LSP, TGW, TTK, TMK, VAQ, TAW, TGS, VKQ, SAP, LSK, LAP, LAQ, TAK, SAK, TGC, TQK, TVA, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG, or a substitution, e.g., a conservative substitution, of any of the aforesaid amino acid sequences;
(ii) LSP; or
(iii) TGW.

21. The AAV capsid variant of any one of claims 1-20, wherein the AAV capsid variant further comprises:
(i) the amino acid V at position 596 numbered according to SEQ ID NO: 138 or at position 603 numbered according to SEQ ID NO: 5, 8, or 3636; the amino acid K, P, E or H at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636;
(ii) the amino acid V at position 596 numbered according to SEQ ID NO: 138 or at position 603 numbered according to SEQ ID NO: 5, 8, or 3636; the amino acid K at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636;
(iii) the amino acid V at position 596 numbered according to SEQ ID NO: 138 or at position 603 numbered according to SEQ ID NO: 5, 8, or 3636; the amino acid P at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636; or
(iv) the amino acid V at position 596 numbered according to SEQ ID NO: 138 or at position 603 numbered according to SEQ ID NO: 5, 8, or 3636; the amino acid Q at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636.

22. The AAV capsid variant of any one of claims 13-21, which further comprises [E], wherein [E] comprises X11, X12, and X13, wherein:
(a) position X11 is: V, D, F, A, E, L, G, or I;
(b) position X12 is: K, P, Q, R, L, H, or E; and
(c) position X13 is: N, H, S, T, P, K, I, D, or Y; or a substitution, e.g., a conservative substitution, of any of the aforesaid amino acids in (a)-(c).

23. The AAV capsid variant of claim 22, wherein [E] comprises:
(i) VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH, or a substitution, e.g., a conservative substitution, of any of the aforesaid amino acid sequences; or
(ii) VKN, VPN, or VQN.

24. The AAV capsid variant of claim 22 or 23, wherein [A][B][C][D][E] comprises:
(i) the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-475, 477-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, 1135;
(ii)
(SEQ ID NO: 566)
PLNGAVHLYAQAQLSPVKN;
(iii)
(SEQ ID NO: 314)
PLNGAVHLYAQAQTGWVPN;
(iv) an amino acid sequence comprising any portion of an amino acid sequence in (i)-(iii), e.g., any 2, 3, 4, 5, 6, 7, 8, 9, 10, 11, 12, 13, 14, 15, 16, 17, or 18 amino acids, e.g., consecutive amino acids, thereof;
(v) an amino acid sequence comprising at least one, two, or three but no more than four substitutions (e.g., conservative substitutions), relative to any of the amino acid sequences in (i)-(iii); or
(vi) an amino acid sequence comprising at least one, two, or three but no more than four different amino acids, relative to any one of the amino acid sequences in (i)-(iii).

25. The AAV capsid variant of any one of claims 22-24, which comprises from N-terminus to C-terminus [A][B][C][D][E], wherein:
(i) [A] is present immediately subsequent to position 586, and replaces positions 587 and 588 (e.g., A587 and Q588), numbered according to SEQ ID NO: 138;
(ii) [B] is present immediately subsequent to [A];
(iii) [C] is present immediately subsequent to position 588, and replaces positions 589-592 (e.g., A589, Q590, A591, Q592), numbered according to the amino acid sequence of SEQ ID NO: 138;
(iv) [D] is present immediately subsequent to position 592, and replaces positions 593-595 (e.g., T593, G594, W595), numbered according to the amino acid sequence of SEQ ID NO: 138;
(v) [E] is present immediately subsequent to position 595, and replaces positions 596-598 (e.g., V596, Q597, N598), numbered according to the amino acid sequence of SEQ ID NO: 138; and/or
(vi) [A][B][C][D][E] is present immediately subsequent to position 586 and replaces positions 587-598 (e.g., A587, Q588, A589, Q590, A591, Q592, T593, G594, W595, V596, Q597, N598), numbered according to SEQ ID NO: 138.

26. The AAV capsid variant of any one of claims 22-25, wherein:
(i) the AAV capsid variant comprises an amino acid other than A at position 587 and/or an amino acid other than Q at position 588, numbered according to SEQ ID NO: 138;
(ii) [A] corresponds to positions 587-591 of SEQ ID NO: 5, 8, or 3636;
(iii) [B] corresponds to positions 592 to 595 of SEQ ID NO: 5, 8, or 3636;
(iv) [C] corresponds to positions 596-599 of SEQ ID NO: 5, 8, or 3636;
(v) [D] corresponds to positions 600-602 of SEQ ID NO: 5, 8, or 3636;
(vi) [E] corresponds to positions 603-605 of SEQ ID NO: 5, 8, or 3636; and/or
(vii) [A][B][C][D][E] corresponds to positions 587-605 of SEQ ID NO: 5, 8, or 3636.

27. The AAV capsid variant of any one of claims 1-26, which comprises:
(i) the amino acid sequence corresponding to positions 203-743, e.g., a VP3, of SEQ ID NO: 8, or a sequence with at least 90%, 95%, 96%, 97%, 98%, or 99% sequence identity thereto
(ii) the amino acid sequence corresponding to positions 138-743, e.g., a VP2, of SEQ ID NO: 8, or a sequence with at least 90%, 95%, 96%, 97%, 98%, or 99% sequence identity thereto;
(iii) the amino acid sequence of SEQ ID NO: 8 (e.g., a VP1), or an amino acid sequence with at least 90%, 95%, 96%, 97%, 98%, or 99% sequence identity thereto;
(iv) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 8; and/or
(v) an amino acid sequence comprising at least one, two or three, but not more than 30, 20 or 10 different amino acids, relative to the amino acid sequence of SEQ ID NO: 8.

28. The AAV capsid variant of any one of claims 1-26, which comprises:
(i) the amino acid sequence corresponding to positions 203-743, e.g., a VP3, of SEQ ID NO: 5, or a sequence with at least 90%, 95%, 96%, 97%, 98%, or 99% sequence identity thereto
(ii) the amino acid sequence corresponding to positions 138-743, e.g., a VP2, of SEQ ID NO: 5, or a sequence with at least 90%, 95%, 96%, 97%, 98%, or 99% sequence identity thereto;
(iii) the amino acid sequence of SEQ ID NO: 5 (e.g., a VP1), or an amino acid sequence with at least 90%, 95%, 96%, 97%, 98%, or 99% sequence identity thereto;
(iv) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 5; and/or
(v) an amino acid sequence comprising at least one, two or three, but not more than 30, 20 or 10 different amino acids, relative to the amino acid sequence of SEQ ID NO: 5.

29. An AAV capsid variant comprising the amino acid P at position 587, the amino acid L at position 588, and the amino acid sequence NGAVHLY (SEQ ID NO: 3689), which is present immediately subsequent to position 588, numbered according to SEQ ID NO: 8; and
wherein the AAV capsid variant further comprises
(a) a VP1 protein comprising the amino acid sequence of SEQ ID NO: 8;
(b) a VP2 protein comprising the amino acid sequence of positions 138-743 of SEQ ID NO: 8;
(c) a VP3 protein comprising the amino acid sequence of positions 203-743 of SEQ ID NO: 8; or
(d) an amino acid sequence with at least 90% (e.g., at least about 95, 96, 97, 98, or 99%) sequence identity to any of the amino acid sequences in (a)-(c); and
wherein the AAV capsid variant does not comprise the amino acid sequence TGW at positions 600-603, numbered according to SEQ ID NO: 8; or the amino acid sequence VQN at positions 603-605, numbered according to SEQ ID NO: 8.

30. An AAV capsid variant comprising the amino acid P at position 587, the amino acid L at position 588, and the amino acid sequence NGAVHLY (SEQ ID NO: 3689), which is present immediately subsequent to position 588, numbered according to SEQ ID NO: 5; and
wherein the AAV capsid variant further comprises
(a) a VP1 protein comprising the amino acid sequence of SEQ ID NO: 5;
(b) a VP2 protein comprising the amino acid sequence of positions 138-743 of SEQ ID NO: 5;
(c) a VP3 protein comprising the amino acid sequence of positions 203-743 of SEQ ID NO: 5; or
(d) an amino acid sequence with at least 90% (e.g., at least about 95, 96, 97, 98, or 99%) sequence identity to any of the amino acid sequences in (a)-(c); and
wherein the AAV capsid variant does not comprise the amino acid sequence TGW at positions 600-603, numbered according to SEQ ID NO: 5; or the amino acid sequence VQN at positions 603-605, numbered according to SEQ ID NO: 5.

31. A polynucleotide encoding the AAV capsid variant of any one of claims 1-30.

32. The polynucleotide of claim 31 which comprises the nucleotide sequence of SEQ ID NO: 4 or 7, or a nucleotide sequence with at least 90% (e.g., at least about 95, 96, 97, 98, or 99%) sequence identity to SEQ ID NO: 4 or 7.

33. A peptide comprising:
(a) the amino acid sequence of any of the sequences provided in Tables 1A, 1B, 10, or 20;
(b) an amino acid sequence comprising at least 3, 4, 5, 6, 7, 8, 9, 10, 11, 12, 13, 14, 15, 16, 17, 18, 19, 20 consecutive amino acids from any one of the sequences provided in Tables 1A, 1B, 10, or 20;
(c) an amino acid sequence comprising at least one, two, or three but no more than four different amino acids relative to the amino acid sequence of any one of the sequences provided in Tables 1A, 1B, 10, or 20; or
(d) an amino acid sequence comprising at least one, two, or three but no more than four modifications, e.g., substitutions (e.g., conservative substitutions), relative to the amino acid sequence of any one of the sequences provided in Tables 1A, 1B, 10, or 20;
wherein the peptide, e.g., at 1-5 peptides, e.g., at least 1, 2, 3, 4, or 5 peptides, are fused or coupled, e.g., conjugated, to an active agent, e.g., a therapeutic agent or a diagnostic agent, optionally wherein:
(i) the at least 1-5, e.g., at least 1, 2, 3, 4, or 5, peptides are present in tandem (e.g., connected directly or indirectly via a linker) or in a multimeric configuration;
(ii) the peptide comprises an amino acid sequence of at least 3, 4, 5, 6, 7, 8, 9, 10, 11, 12, 15, 20, 25, 30, or 35 amino acids in length;
(iii) the peptide is present N-terminal relative to the active agent;
(iv) the peptide is present C-terminal relative to the active agent;
(v) the active agent is or comprises a therapeutic agent chosen from a protein (e.g., an enzyme), an antibody molecule, a nucleic acid molecule (e.g., an RNAi agent), or a small molecule;
(vi) the active agent is or comprises a ribonucleic acid complex (e.g., a Cas9/gRNA complex), a plasmid, a closed-end DNA, a circ-RNA, or an mRNA;
(vii) the active agent is an RNAi agent (e.g., a dsRNA, a siRNA, a shRNA, a pre-miRNA, a pri-miRNA, a miRNA, a stRNA, a lncRNA, a piRNA, an antisense oligonucleotide agent (ASO), or a snoRNA, optionally wherein the RNAi agent is an siRNA or an ASO, which optionally comprises at least one modified nucleotide);
(viii) the active agent modulates, e.g., inhibits, decreases or increases, expression of, a CNS related gene, mRNA, and/or protein; and/or
(ix) the active agent is a diagnostic agent is or comprises an imaging agent (e.g., a protein or small molecule compound coupled to a detectable moiety).

34. An AAV particle comprising the AAV capsid variant of any one of claims 1-30, or an AAV capsid variant encoded by the polynucleotide of claim 31 or 32.

35. The AAV particle of claim 34, which comprises a nucleotide sequence encoding a payload, optionally wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.

36. The AAV particle of claim 35, wherein:
(i) the therapeutic protein or functional variant thereof, e.g., a recombinant protein, is associated with (e.g., aberrantly expressed in) a neurological or neurodegenerative disorder, a muscular or neuromuscular disorder, or a neuro-oncological disorder, optionally wherein the therapeutic protein or functional variant thereof is chosen from apolipoprotein E (APOE) (e.g., ApoE2, ApoE3 and/or ApoE4); human survival of motor neuron (SMN) 1 or SMN2; glucocerebrosidase (GBA1); aromatic L-amino acid decarboxylase (AADC); aspartoacylase (ASPA); tripeptidyl peptidase I (CLN2); beta-galactosidase (GLB1); N-sulphoglucosamine sulphohydrolase (SGSH); N-acetyl-alpha-glucosaminidase (NAGLU); iduronate 2-sulfatase (IDS); intracellular cholesterol transporter (NPC1); gigaxonin (GAN); or a combination thereof;
(ii) the antibody or antibody binding fragment binds to
(a) a CNS related target, e.g. an antigen associated with a neurological or neurodegenerative disorder, e.g., β-amyloid, APOE, tau, SOD1, TDP-43, huntingtin (HTT), and/or synuclein;
(b) a muscular or neuromuscular related target, e.g., an antigen associated with a muscular or neuromuscular disorder; or
(c) a neuro-oncology related target, e.g., an antigen associated with a neuro-oncological disorder, e.g., HER2, or EGFR (e.g., EGFRvIII);
(iii) the enzyme comprises a meganuclease, a zinc finger nuclease, a TALEN, a recombinase, integrase, a base editor, a Cas9, or a fragment thereof;
(iv) the component of a gene editing system comprises one or more components of a CRISPR-Cas system, optionally wherein the one or more components of the CRISPR-Cas system comprises a Cas9, e.g., a Cas9 ortholog or a Cpf1, and a single guide RNA (sgRNA), wherein:
(a) the sgRNA is located upstream (5′) of the cas9 enzyme; and/or
(b) the sgRNA is located downstream (3′) of the cas9 enzyme; and/or
(v) the RNAi agent (e.g., a dsRNA, siRNA, shRNA, pre-miRNA, pri-miRNA, miRNA, stRNA, lncRNA, piRNA, or snoRNA), modulates, e.g., inhibits, expression of, a CNS related gene, mRNA, and/or protein, optionally wherein the CNS related gene is chosen from SOD1, MAPT, APOE, HTT, C9ORF72, TDP-43, APP, BACE, SNCA, ATXN1, ATXN3, ATXN7, SCN1A-SCN5A, SCN8A-SCN11A, or a combination thereof.

37. The AAV particle of any one of claims 34-36, which comprises a viral genome comprising a promoter operably linked to the nucleic acid sequence encoding the payload, optionally wherein:
(i) the promoter is chosen from human elongation factor 1α-subunit (EF1α), cytomegalovirus (CMV) immediate-early enhancer and/or promoter, chicken β-actin (CBA) and its derivative CAG, P glucuronidase (GUSB), or ubiquitin C (UBC), neuron-specific enolase (NSE), platelet-derived growth factor (PDGF), platelet-derived growth factor B-chain (PDGF-$), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof;
(ii) the promoter is an EF-1α promoter variant, e.g., a truncated EF-1α promoter; or
(iii) the promoter comprises the nucleotide sequence of any one of SEQ ID NOs: 19, 20, 22, 23, 27, 28, 30-39 or any one of the sequences provided in Table 12, a nucleotide sequence comprising at least one, two, or three but no more than four modifications, e.g., substitutions (e.g., conservative substitutions), relative to the nucleotide sequence of SEQ ID NOs: 19, 20, 22, 23, 27, 28, 30-39 or any one of the sequences provided in Table 12 or any one of the sequences provided in Table 8, or a nucleotide sequence with at least 80% (e.g., 85%, 90%, 95%, 96%, 97%, 98%, or 99%) sequence identity to any one of SEQ ID NOs: 19, 20, 22, 23, 27, 28, 30-39 or any one of the sequences provided in Table 12.

38. The AAV particle of claim 35, wherein the viral genome further comprises:
(i) a polyA signal sequence;
(ii) an inverted terminal repeat (ITR) sequence, optionally wherein the ITR sequence is positioned 5′ relative to the encoded payload and/or the ITR sequence is positioned 3′ relative to the encoded payload;
(iii) an enhancer, a Kozak sequence, an intron region, and/or an exon region;
(iv) a nucleotide sequence encoding a miR binding site, e.g., a miR binding site that modulates, e.g., reduces, expression of the antibody molecule encoded by the viral genome in a cell or tissue where the corresponding miRNA is expressed, optionally wherein the encoded miR binding site modulates, e.g., reduces, expression of the encoded antibody molecule in a cell or tissue of the DRG, liver, heart, hematopoietic lineage, or a combination thereof; and/or
(v) a nucleotide sequence encoding a Rep protein, e.g., a non-structural protein, wherein the Rep protein comprises a Rep78 protein, a Rep68, Rep52 protein, and/or a Rep40 protein, optionally wherein the Rep78 protein, the Rep68 protein, the Rep52 protein, and/or the Rep40 protein are encoded by at least one Rep gene.
the viral genome further comprises a polyA signal sequence.

39. The AAV particle of claim 38, wherein the viral genome comprises:
(i) at least 1-5 copies of the encoded miR binding site, e.g., at least 1, 2, 3, 4, or 5 copies;
(ii) at least 3 copies of an encoded miR binding sites, optionally wherein:
(a) all three copies comprise the same miR binding site, or at least one, two, three, or all of the copies comprise a different miR binding site; and/or
(b) the 3 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence of GATAGTTA; or
(iii) at least 4 copies of an encoded miR binding site, optionally wherein
(a) all four copies comprise the same miR binding site, or at least one, two, three, or all of the copies comprise a different miR binding site; and/or
(b) the 4 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to the nucleotide sequence of GATAGTTA.

40. The AAV particle of claim 38 or 39, wherein the encoded miR binding site comprises a miR122 binding site, a miR183 binding site, a miR-1 binding site, a miR-142-3p, or a combination thereof, optionally wherein:
(i) the encoded miR122 binding site comprises the nucleotide sequence of SEQ ID NO: 3672, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
(ii) the encoded miR183 binding site comprises the nucleotide sequence of SEQ ID NO: 3675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
(iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 4679; and/or
(iv) the encoded miR-142-3p binding site comprises the nucleotide sequence of SEQ ID NO: 3674, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3674.

41. The AAV particle of any one of claims 37-40, wherein the viral genome:
(i) is single stranded;
(ii) is self-complementary; and/or
(ii) further comprises a nucleic acid encoding the AAV capsid variant of any one of claims 1-30.

42. The AAV capsid variant, polynucleotide, peptide, or AAV particle of any one of the preceding claims which is isolated, e.g., recombinant.

43. A vector comprising a polynucleotide encoding the AAV capsid variant of any one of claims 1-30 or 4, or the polynucleotide of any one of claims 31, 32, or 42.

44. A cell, e.g., a host cell, comprising the AAV capsid variant of any one of claims 1-30 or 42, the polynucleotide of any one of claim 31, 32, or 42, the peptide of claim 33 or 42, the AAV particle of any one of claims 34-42, or the vector of claim 43, optionally wherein:
(i) the cell is a mammalian cell or an insect cell;
(ii) the cell is a cell of a brain region or a spinal cord region, optionally a cell of the brain stem, hippocampus, or thalamus; and/or
(iii) the cell is a neuron, a sensory neuron, a motor neuron, an astrocyte, a glial cell, oligodendrocyte, or a muscle cell (e.g., a cell of the heart, diaphragm, or quadriceps).

45. A method of making an AAV particle, comprising
(i) providing a host cell comprising a viral genome; and
(ii) incubating the host cell under conditions suitable to enclose the viral genome in the AAV capsid variant of any one of claims 1-30 or 42, or an AAV capsid variant encoded by the polynucleotide of any one of claims 31, 32, or 42;
thereby making the AAV particle.

46. A pharmaceutical composition comprising the AAV particle of any one of claims 34-42, an AAV particle comprising the AAV capsid variant of any one of claims 1-30 or 42, and a pharmaceutically acceptable excipient.

47. A pharmaceutical formulation comprising the AAV particle of any one of claims 34-42, an AAV particle comprising the AAV capsid variant of any one of claims 1-30 or 42; and further comprising:
(i) a buffering agent (e.g., a tris base); and a polyether, e.g., one or more polyethers (e.g., glycerol, glycerin, and polyethylene glycol (e.g., low-molecular-weight PEG));
(ii) a buffering agent (e.g., a tris base); a polyether (e.g., glycerol, glycerin, and polyethylene glycol (e.g., low-molecular-weight PEG)); a salt (e.g., sodium chloride, potassium chloride, or magnesium chloride); and a surfactant (e.g., an ethylene oxide/propylene oxide copolymer (e.g., poloxamers such as Pluronic® F-68 or F-127)), or a zwitterionic surfactant, wherein the concentration of the surfactant, e.g., Pluronic F-68, is between 0.0005-0.0015% (e.g., 0.0008-0.0012%, e.g., 0.001% or about 0.001%));
(iii) a buffering agent (e.g., a tris base); a sugar (e.g., trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose); a salt (e.g., sodium chloride, potassium chloride, or magnesium chloride); and a surfactant (e.g., an ethylene oxide/propylene oxide copolymer (e.g., poloxamers such as Pluronic® F-68 or F-127)), or a zwitterionic surfactant, wherein the concentration of the surfactant, e.g., Pluronic F-68, is between 0.0005-0.0015% (e.g., 0.0008-0.0012%, e.g., 0.001% or about 0.001%)); or
(iv) a buffering agent (e.g., a tris base); and a sugar (e.g., trehalose, sucrose, lactulose, lactose, maltose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, or xylobiose);
optionally wherein the AAV particle is present at a dose of:
(a) 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg;
(b) about 6.7e10 VG/kg to 6.7e12 VG/kg, about 1.3e11 VG/kg to 3.4e12 VG/kg, or about 2.2e11 VG/kg to 2e12 VG/kg;
(c) about 4e11 VG/kg to 8e11 VG/kg (e.g., about 6.7e11 VG/kg);
(d) about 2e11 VG/kg to 2e13 VG/kg, about 4e11 VG/kg to 1e13 VG/kg, about 6.7e11 VG/kg to about 6e12 VG/kg;
(e) about 1e12 VG/kg to 5e12 VG/kg (e.g., about 2e12 VG/kg);
(f) about 6.7e11 VG/kg to 6.7e13 VG/kg, about 1.3e12 VG/kg to 3.4e13 VG/kg, or about 2.2e12 VG/kg to 2e13 VG/kg;
(g) about 4e12 VG/kg to 8e12 VG/kg (e.g., about 6.7e12 VG/kg);
(h) about 2e12 VG/kg to 2e14 VG/kg, about 4e12 VG/kg to 1e14 VG/kg, about 6.7e12 VG/kg to about 6e13 VG/kg; or
(i) about 1e13 VG/kg to 5e13 VG/kg (e.g., about 2e13 VG/kg).

48. A method of delivering a payload to a cell or tissue (e.g., a CNS cell or a CNS tissue), comprising administering an effective amount of the pharmaceutical composition of claim 46, the pharmaceutical formulation of claim 47, or the AAV particle of any one of claims 34-42, or an AAV particle comprising the capsid variant of any one of claims 1-30 or 42.

49. The method of claim 48, wherein the cell is:
(i) a cell of a brain region or a spinal cord region, optionally a cell of the frontal cortex, sensory cortex, motor cortex, caudate, cerebellar cortex, cerebral cortex, brain stem, hippocampus, or thalamus;
(ii) a neuron, a sensory neuron, a motor neuron, an astrocyte, a glial cell, or an oligodendrocyte; and/or
(iii) within a subject, optionally wherein the subject has, has been diagnosed with having, or is at risk of having a neurological disorder, e.g., a neurodegenerative disorder, a neuro-oncological disorder, a muscular disorder, or a neuromuscular disorder.

50. A method of treating a subject having or diagnosed with having a neurological disorder, e.g., a neurodegenerative disorder, a neuro-oncological disorder, a muscular disorder, or a neuromuscular disorder, comprising administering to the subject an effective amount of the pharmaceutical composition of claim 46, the pharmaceutical formulation of claim 47, the AAV particle of any one of claims 34-42, or an AAV particle comprising the capsid variant of any one of claims 1-30 or 42.

51. The method of claim 49 or 50, wherein the neurological disorder, neurodegenerative disorder, muscular disorder, neuromuscular disorder, or neuro-oncological disorder is Huntington's Disease, Amyotrophic Lateral Sclerosis (ALS), Gaucher Disease, Dementia with Lewy Bodies, Parkinson's disease, Spinal Muscular Atrophy, Alzheimer's Disease, a leukodystrophy (e.g., Alexander disease, autosomal dominant leukodystrophy with autonomic diseases (ADLD), Canavan disease, cerebrotendinous xanthomatosis (CTX), metachromatic leukodystrophy (MLD), Pelizaeus-Merzbacher disease, or Refsum disease), or a cancer (e.g., a HER2/neu positive cancer or a glioblastoma).

52. The method of claim 50 or 51, where treating comprises prevention of progression of the disease or disorder in the subject, optionally wherein the subject is a human.

53. The method of any one of claims 49-52, wherein the AAV particle is administered to the subject:
(i) intravenously, via intra-cisterna magna injection (ICM), intracerebrally, intrathecally, intracerebroventricularly, via intraparenchymal administration, or intramuscularly;
(ii) via focused ultrasound (FUS), e.g., coupled with the intravenous administration of microbubbles (FUS-MB), or MRI-guided FUS coupled with intravenous administration; or
(iii) intravenously.

54. The method of any one of claims 49-53, wherein the wherein the AAV particle is administered to the subject at a dose of:
(a) about 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg;
(b) about 6.7e10 VG/kg to 6.7e12 VG/kg, about 1.3e11 VG/kg to 3.4e12 VG/kg, or about 2.2e11 VG/kg to 2e12 VG/kg;
(c) about 4e11 VG/kg to 8e11 VG/kg (e.g., about 6.7e11 VG/kg);
(d) about 2e11 VG/kg to 2e13 VG/kg, about 4e11 VG/kg to 1e13 VG/kg, about 6.7e11 VG/kg to about 6e12 VG/kg;
(e) about 1e12 VG/kg to 5e12 VG/kg (e.g., about 2e12 VG/kg);
(f) about 6.7e11 VG/kg to 6.7e13 VG/kg, about 1.3e12 VG/kg to 3.4e13 VG/kg, or about 2.2e12 VG/kg to 2e13 VG/kg;
(g) about 4e12 VG/kg to 8e12 VG/kg (e.g., about 6.7e12 VG/kg);
(h) about 2e12 VG/kg to 2e14 VG/kg, about 4e12 VG/kg to 1e14 VG/kg, about 6.7e12 VG/kg to about 6e13 VG/kg; or
(i) about 1e13 VG/kg to 5e13 VG/kg (e.g., about 2e13 VG/kg).

55. The pharmaceutical composition of claim 46, the pharmaceutical formulation of claim 47, the AAV particle of any one of claims 34-42, or an AAV particle comprising the capsid variant of any one of claims 1-30 or 42, for use in a method of delivering a payload to a cell or tissue.

56. The pharmaceutical composition of claim 46, the pharmaceutical formulation of claim 47, the AAV particle of any one of claims 34-42, or an AAV particle comprising the capsid variant of any one of claims 1-30 or 42, for use in a method of treating a neurological disorder, a neurodegenerative disorder, a muscular disorder, a neuromuscular disorder, or a neuro-oncological disorder.

57. The pharmaceutical composition of claim 46, the pharmaceutical formulation of claim 47, the AAV particle of any one of claims 34-42, or an AAV particle comprising the capsid variant of any one of claims 1-30 or 42, for use in the manufacture of a medicament.

58. Use of the pharmaceutical composition of claim 46, the pharmaceutical formulation of claim 47, the AAV particle of any one of claims 34-42, or an AAV particle comprising the capsid variant of any one of claims 1-30 or 42, in the manufacture of a medicament for treating a neurological disorder, a neurodegenerative disorder, a muscular disorder, a neuromuscular disorder, or a neuro-oncological disorder.

59. Use of the pharmaceutical composition of claim 46, the pharmaceutical formulation of claim 47, or the AAV particle of any one of claims 34-42, an AAV particle comprising the capsid variant of any one of claims 1-30 or 4, in the manufacture of a medicament.
